SEQUENCE LISTING (107 sequences)

SEQ ID NO: 1 - RNA, 21 nt, synthetic construct
  source 1..21: mol_type = other RNA
cattttaatcctcactctaaa

SEQ ID NO: 2 - RNA, 23 nt, synthetic construct
  source 1..23: mol_type = other RNA
tttagagtgaggattaaaatgag

SEQ ID NO: 3 - RNA, 21 nt, synthetic construct
  source 1..21: mol_type = other RNA
tactgttggattgattcgaaa

SEQ ID NO: 4 - RNA, 23 nt, synthetic construct
  source 1..23: mol_type = other RNA
tttcgaatcaatccaacagtagc

SEQ ID NO: 5 - RNA, 45 nt, synthetic construct
  source 1..45: mol_type = other RNA
  modified_base 22: mod_base = OTHER; note = 5'-methyluridine-3'-phosphate
  modified_base 23: mod_base = OTHER; note = 5'-methyluridine-3'-phosphate
  modified_base 24: mod_base = OTHER; note = 5'-methyluridine-3'-phosphate
tactgttggattgattcgaaatttcattttaatcctcactctaaa

SEQ ID NO: 6 - RNA, 45 nt, synthetic construct; identical to SEQ ID NO: 5
  source 1..45: mol_type = other RNA

SEQ ID NO: 7 - RNA, 45 nt, synthetic construct; identical to SEQ ID NO: 5
  source 1..45: mol_type = other RNA

SEQ ID NO: 8 - RNA, 45 nt, synthetic construct; identical to SEQ ID NO: 5
  source 1..45: mol_type = other RNA

SEQ ID NO: 9 - RNA, 45 nt, synthetic construct; identical to SEQ ID NO: 5
  source 1..45: mol_type = other RNA
  modified_base 22: mod_base = OTHER; note = 5'-methyluridine-3'-phosphate
  modified_base 23: mod_base = OTHER; note = 5'-methyluridine-3'-phosphate
  modified_base 24: mod_base = OTHER; note = 5'-methyluridine-3'-phosphate

SEQ ID NO: 10 - RNA, 45 nt, synthetic construct
  source 1..45: mol_type = other RNA
  modified_base 22: mod_base = OTHER; note = 5'-methyluridine-3'-phosphate
  modified_base 23: mod_base = OTHER; note = 5'-methyluridine-3'-phosphate
  modified_base 24: mod_base = OTHER; note = 5'-methyluridine-3'-phosphate
cattttaatcctcactctaaattttactgttggattgattcgaaa

SEQ ID NO: 11 - RNA, 45 nt, synthetic construct; identical to SEQ ID NO: 10
  source 1..45: mol_type = other RNA

SEQ ID NO: 12 - RNA, 45 nt, synthetic construct; identical to SEQ ID NO: 5
  source 1..45: mol_type = other RNA
  modified_base 22: mod_base = OTHER; note = 5'-methyluridine-3'-phosphate
  modified_base 23: mod_base = OTHER; note = 5'-methyluridine-3'-phosphate
  modified_base 24: mod_base = OTHER; note = 5'-methyluridine-3'-phosphate

SEQ ID NO: 13 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 14 - RNA, 23 nt, synthetic construct
  source 1..23: mol_type = other RNA
  modified_base 1: mod_base = OTHER; note = 2'-O-methyluridine-3'-phosphate 5'-(E)-Vinylphosphonate
  modified_base 2: mod_base = OTHER; note = 2'-deoxy-2'-fluorouridine-3'-phosphate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 15 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
tactgttggattgattcgaaa

SEQ ID NO: 16 - RNA, 23 nt, synthetic construct
  source 1..23: mol_type = other RNA
  modified_base 1: mod_base = OTHER; note = 2'-O-methyluridine-3'-phosphate 5'-(E)-Vinylphosphonate
  modified_base 2: mod_base = OTHER; note = 2'-deoxy-2'-fluorouridine-3'-phosphate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluorocytidine-3'-phosphate
  modified_base 15: mod_base = OTHER; note = 2'-O-methyladenosine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
tttcgaatcaatccaacagtagc

SEQ ID NO: 17 - DNA, 45 bp, synthetic construct
  source 1..45: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
  misc_feature 1..21: note = RNA
  misc_feature 25..45: note = RNA
  misc_feature 22..24: note = DNA
tactgttggattgattcgaaatttcattttaatcctcactctaaa

SEQ ID NO: 18 - RNA, 45 nt, synthetic construct; identical to SEQ ID NO: 5
  source 1..45: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-O-methyluridine-3'-phosphate
  modified_base 23: mod_base = OTHER; note = 2'-O-methyluridine-3'-phosphate
  modified_base 24: mod_base = OTHER; note = 2'-O-methyluridine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine

SEQ ID NO: 19 - RNA, 45 nt, synthetic construct; identical to SEQ ID NO: 5
  source 1..45: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine

SEQ ID NO: 20 - RNA, 45 nt, synthetic construct; identical to SEQ ID NO: 5
  source 1..45: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-deoxy-2'-fluorouridine-3'-phosphate
  modified_base 23: mod_base = OTHER; note = 2'-deoxy-2'-fluorouridine-3'-phosphate
  modified_base 24: mod_base = OTHER; note = 2'-deoxy-2'-fluorouridine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine

SEQ ID NO: 21 - DNA, 45 bp, synthetic construct; identical to SEQ ID NO: 17
  source 1..45: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
  misc_feature 1..21: note = RNA
  misc_feature 25..45: note = RNA
  misc_feature 22..24: note = DNA

SEQ ID NO: 22 - DNA, 45 bp, synthetic construct
  source 1..45: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methyluridine-3'-phosphorothioate
  modified_base 26: mod_base = OTHER; note = 2'-O-methyladenosine-3'-phosphorothioate
  modified_base 27: mod_base = OTHER; note = 2'-O-methylcytidine-3'-phosphate
  modified_base 28: mod_base = OTHER; note = 2'-O-methyluridine-3'-phosphate
  modified_base 29: mod_base = OTHER; note = 2'-O-methylguanosine-3'-phosphate
  modified_base 30: mod_base = OTHER; note = 2'-O-hexadecyl-uridine-3'-phosphate
  modified_base 31: mod_base = OTHER; note = 2'-deoxy-2'-fluorouridine-3'-phosphate
  modified_base 32: mod_base = OTHER; note = 2'-O-methylguanosine-3'-phosphate
  modified_base 33: mod_base = OTHER; note = 2'-deoxy-2'-fluoroguanosine-3'-phosphate
  modified_base 34: mod_base = OTHER; note = 2'-deoxy-2'-fluoroadenosine-3'-phosphate
  modified_base 35: mod_base = OTHER; note = 2'-deoxy-2'-fluorouridine-3'-phosphate
  modified_base 36: mod_base = OTHER; note = 2'-O-methyluridine-3'-phosphate
  modified_base 37: mod_base = OTHER; note = 2'-O-methylguanosine-3'-phosphate
  modified_base 38: mod_base = OTHER; note = 2'-O-methyladenosine-3'-phosphate
  modified_base 39: mod_base = OTHER; note = 2'-O-methyluridine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methylguanosine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
  misc_feature 1..21: note = RNA
  misc_feature 25..45: note = RNA
  misc_feature 22..24: note = DNA
cattttaatcctcactctaaattttactgttggattgattcgaaa

SEQ ID NO: 23 - RNA, 45 nt, synthetic construct; identical to SEQ ID NO: 10
  source 1..45: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-O-methyluridine-3'-phosphate
  modified_base 23: mod_base = OTHER; note = 2'-O-methyluridine-3'-phosphate
  modified_base 24: mod_base = OTHER; note = 2'-O-methyluridine-3'-phosphate
  modified_base 25: mod_base = OTHER; note = 2'-O-methyluridine-3'-phosphorothioate
  modified_base 26: mod_base = OTHER; note = 2'-O-methyladenosine-3'-phosphorothioate
  modified_base 27: mod_base = OTHER; note = 2'-O-methylcytidine-3'-phosphate
  modified_base 28: mod_base = OTHER; note = 2'-O-methyluridine-3'-phosphate
  modified_base 29: mod_base = OTHER; note = 2'-O-methylguanosine-3'-phosphate
  modified_base 30: mod_base = OTHER; note = 2'-O-hexadecyl-uridine-3'-phosphate
  modified_base 31: mod_base = OTHER; note = 2'-deoxy-2'-fluorouridine-3'-phosphate
  modified_base 32: mod_base = OTHER; note = 2'-O-methylguanosine-3'-phosphate
  modified_base 33: mod_base = OTHER; note = 2'-deoxy-2'-fluoroguanosine-3'-phosphate
  modified_base 34: mod_base = OTHER; note = 2'-deoxy-2'-fluoroadenosine-3'-phosphate
  modified_base 35: mod_base = OTHER; note = 2'-deoxy-2'-fluorouridine-3'-phosphate
  modified_base 36: mod_base = OTHER; note = 2'-O-methyluridine-3'-phosphate
  modified_base 37: mod_base = OTHER; note = 2'-O-methylguanosine-3'-phosphate
  modified_base 38: mod_base = OTHER; note = 2'-O-methyladenosine-3'-phosphate
  modified_base 39: mod_base = OTHER; note = 2'-O-methyluridine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methylguanosine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine

SEQ ID NO: 24 - DNA, 45 bp, synthetic construct; identical to SEQ ID NO: 17
  source 1..45: mol_type = other DNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-methyluridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
  misc_feature 1..21: note = RNA
  misc_feature 25..45: note = RNA
  misc_feature 22..24: note = DNA

SEQ ID NO: 25 - DNA, 20 bp, synthetic construct
  source 1..20: mol_type = other DNA
tttttttttttttttttttt

SEQ ID NO: 26 - DNA, 20 bp, synthetic construct
  source 1..20: mol_type = other DNA
  modified_base 20: mod_base = OTHER; note = 2'-deoxyadenosine-3'-phosphate
aaaaaaaaaaaaaaaaaaaa

SEQ ID NO: 27 - RNA, 20 nt, synthetic construct
  source 1..20: mol_type = other RNA
  modified_base 1: mod_base = OTHER; note = 5'-methyluridine-3'-phosphate
  modified_base 2: mod_base = OTHER; note = 5'-methyluridine-3'-phosphate
  modified_base 3: mod_base = OTHER; note = 5'-methyluridine-3'-phosphate
  modified_base 4: mod_base = OTHER; note = 5'-methyluridine-3'-phosphate
  modified_base 5: mod_base = OTHER; note = 5'-methyluridine-3'-phosphate
  modified_base 6: mod_base = OTHER; note = 5'-methyluridine-3'-phosphate
  modified_base 7: mod_base = OTHER; note = 5'-methyluridine-3'-phosphate
  modified_base 8: mod_base = OTHER; note = 5'-methyluridine-3'-phosphate
  modified_base 9: mod_base = OTHER; note = 5'-methyluridine-3'-phosphate
  modified_base 10: mod_base = OTHER; note = 5'-methyluridine-3'-phosphate
  modified_base 11: mod_base = OTHER; note = 5'-methyluridine-3'-phosphate
  modified_base 12: mod_base = OTHER; note = 5'-methyluridine-3'-phosphate
  modified_base 13: mod_base = OTHER; note = 5'-methyluridine-3'-phosphate
  modified_base 14: mod_base = OTHER; note = 5'-methyluridine-3'-phosphate
  modified_base 15: mod_base = OTHER; note = 5'-methyluridine-3'-phosphate
  modified_base 16: mod_base = OTHER; note = 5'-methyluridine-3'-phosphate
  modified_base 17: mod_base = OTHER; note = 5'-methyluridine-3'-phosphate
  modified_base 18: mod_base = OTHER; note = 5'-methyluridine-3'-phosphate
  modified_base 19: mod_base = OTHER; note = 5'-methyluridine-3'-phosphate
  modified_base 20: mod_base = OTHER; note = 5'-methyluridine
tttttttttttttttttttt

SEQ ID NO: 28 - RNA, 20 nt, synthetic construct
  source 1..20: mol_type = other RNA
aaaaaaaaaaaaaaaaaaaa

SEQ ID NO: 29 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 30 - RNA, 21 nt, synthetic construct
  source 1..21: mol_type = other RNA
cattttaatcctcactctaaa

SEQ ID NO: 31 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 32 - RNA, 23 nt, synthetic construct
  source 1..23: mol_type = other RNA
tttagagtgaggattaaaatgag

SEQ ID NO: 33 - RNA, 45 nt, synthetic construct
  source 1..45: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-deoxy-2'-fluoroguanosine-3'-phosphate
  modified_base 23: mod_base = OTHER; note = 2'-deoxy-2'-fluoroadenosine-3'-phosphate
  modified_base 24: mod_base = OTHER; note = 2'-deoxy-2'-fluorouridine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
cattttaatcctcactctaaagatcattttaatcctcactctaaa

SEQ ID NO: 34 - DNA, 45 bp, synthetic construct
  source 1..45: mol_type = other DNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-hexadecyl-uridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-methyladenosine
  misc_feature 1..21: note = RNA
  misc_feature 25..45: note = RNA
  misc_feature 22..24: note = DNA
cattttaatcctcactctaaagatcattttaatcctcactctaaa

SEQ ID NO: 35 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate connected through the 3'-O to the 5'-O end of another nucleotide via a Q315Q315Q315 linker
cattttaatcctcactctaaa

SEQ ID NO: 36 - RNA, 21 nt, synthetic construct
  source 1..21: mol_type = other RNA
  modified_base 1: mod_base = OTHER; note = 2'-O-methylcytidine-3'-phosphorothioate connected through the 5'-O to the 3'-O end of another nucleotide via a Q315Q315Q315 linker
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroadenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
cattttaatcctcactctaaa

SEQ ID NO: 37 - protein, 8 aa, unidentified
GRGDSPKC

SEQ ID NO: 38 - protein, 5 aa, unidentified
  SITE 4: note = D-phenylalanine
  REGION 1..5: note = cyclic
RGDFC

SEQ ID NO: 39 - protein, 29 aa, unidentified
AALEALAEALEALAEALEALAEAAAAGGC

SEQ ID NO: 40 - protein, 30 aa, unidentified
AALAEALAEALAEALAEALAEALAAAAGGC

SEQ ID NO: 41 - protein, 15 aa, unidentified
ALEALAEALEALAEA

SEQ ID NO: 42 - protein, 22 aa, unidentified
GLFEAIEGFIENGWEGMIWDYG

SEQ ID NO: 43 - protein, 23 aa, unidentified
GLFGAIAGFIENGWEGMIDGWYG

SEQ ID NO: 44 - protein, 43 aa, unidentified
GLFEAIEGFIENGWEGMIDGWYGCGLFEAIEGFIENGWEGMID

SEQ ID NO: 45 - protein, 44 aa, unidentified
GLFEAIEGFIENGWEGMIDGGCGLFEAIEGFIENGWEGMIDGGC

SEQ ID NO: 46 - protein, 35 aa, unidentified
GLFGALAEALAEALAEHLAEALAEALEALAAGGSC

SEQ ID NO: 47 - protein, 34 aa, unidentified
GLFEAIEGFIENGWEGLAEALAEALEALAAGGSC

SEQ ID NO: 48 - protein, 41 aa, unidentified
  SITE 17: note = Norleucine
  SITE 38: note = Norleucine
GLFEAIEGFIENGWEGXIDGKGLFEAIEGFIENGWEGXIDG

SEQ ID NO: 49 - protein, 19 aa, unidentified
LFEALLELLESLWELLLEA

SEQ ID NO: 50 - protein, 20 aa, unidentified
GLFKALLKLLKSLWKLLLKA

SEQ ID NO: 51 - protein, 20 aa, unidentified
GLFRALLRLLRSLWRLLLRA

SEQ ID NO: 52 - protein, 30 aa, unidentified
WEAKLAKALAKALAKHLAKALAKALKACEA

SEQ ID NO: 53 - protein, 22 aa, unidentified
GLFFEAIAEFIEGGWEGLIEGC

SEQ ID NO: 54 - protein, 26 aa, unidentified
GIGAVLKVLTTGLPALISWIKRKRQQ

SEQ ID NO: 55 - protein, 8 aa, unidentified
HHHHHWYG

SEQ ID NO: 56 - protein, 10 aa, unidentified
CHKKKKKKHC

SEQ ID NO: 57 - protein, 16 aa, unidentified
RQIKIWFQNRRMKWKK

SEQ ID NO: 58 - protein, 14 aa, unidentified
GRKKRRQRRRPPQC

SEQ ID NO: 59 - protein, 27 aa, unidentified
GALFLGWLGAAGSTMGAWSQPKKKRKV

SEQ ID NO: 60 - protein, 18 aa, unidentified
LLIILRRRIRKQAHAHSK

SEQ ID NO: 61 - protein, 26 aa, unidentified
GWTLNSAGYLLKINLKALAALAKKIL

SEQ ID NO: 62 - protein, 18 aa, unidentified
KLALKLALKALKAALKLA

SEQ ID NO: 63 - protein, 9 aa, unidentified
RRRRRRRRR

SEQ ID NO: 64 - protein, 10 aa, unidentified
KFFKFFKFFK

SEQ ID NO: 65 - protein, 37 aa, unidentified
LLGDFFRKSKEKIGKEFKRIVQRIKDFLRNLVPRTES

SEQ ID NO: 66 - protein, 31 aa, unidentified
SWLSKTAKKLENSAKKRISEGIAIAIQGGPR

SEQ ID NO: 67 - protein, 30 aa, unidentified
ACYCRIPACIAGERRYGTCIYQGRLWAFCC

SEQ ID NO: 68 - protein, 36 aa, unidentified
DHYNCVSSGGQCLYSACPIFTKIQGTCYRGKAKCCK

SEQ ID NO: 69 - protein, 42 aa, unidentified
  SITE 42: note = C-terminal amide
RRRPRPPYLPRPRPPPFFPPRLPPRIPPGFPPRFPPRFPGKR

SEQ ID NO: 70 - protein, 13 aa, unidentified
  SITE 13: note = C-terminal amide
ILPWKWPWWPWRR

SEQ ID NO: 71 - protein, 16 aa, unidentified
AAVALLPAVLLALLAP

SEQ ID NO: 72 - protein, 11 aa, unidentified
AALLPVLLAAP

SEQ ID NO: 73 - protein, 12 aa, unidentified
RKCRIVVIRVCR

SEQ ID NO: 74 - DNA, 20 bp, synthetic construct
  source 1..20: mol_type = other DNA
  modified_base 19: mod_base = OTHER; note = 2'-deoxythymidine-3'-phosphorothioate
tttttttttttttttttttt

SEQ ID NO: 75 - DNA, 20 bp, synthetic construct
  source 1..20: mol_type = other DNA
  modified_base 1: mod_base = OTHER; note = 2'-deoxythymidine-3'-phosphorothioate
tttttttttttttttttttt

SEQ ID NO: 76 - DNA, 23 bp, synthetic construct
  source 1..23: mol_type = other DNA
  modified_base 1: mod_base = OTHER; note = 2'-deoxythymidine-3'-phosphate 5'-phosphate 6-azidohex-1-yl ester
  modified_base 23: mod_base = OTHER; note = 2'-deoxythymidine-3'-phosphate ((2S,4R)-N-(6-(1-hex -5-yn-1-onylamino)-1-hexan-1-onyl)-4-hydroxypyrrolidin-2-y l)methyl ester
ttttttttttttttttttttttt

SEQ ID NO: 77 - DNA, 23 bp, synthetic construct
  source 1..23: mol_type = other DNA
  modified_base 1: mod_base = OTHER; note = 2'-deoxythymidine-3'-phosphate 5'-phosphate 6-azidohex-1-yl ester
  modified_base 23: mod_base = OTHER; note = 2'-deoxythymidine-3'-phosphate ((2S,4R)-N-(6-(1-hex -5-yn-1-onylamino)-1-hexan-1-onyl)-4-hydroxypyrrolidin-2-y l)methyl ester
  misc_RNA 1..23: note = cyclic structure formed by trizaole-forming click cyclization of 5'-terminal azide with the 3'-terminal alkyne
ttttttttttttttttttttttt

SEQ ID NO: 78 - RNA, 23 nt, synthetic construct
  source 1..23: mol_type = other RNA
  modified_base 1: mod_base = OTHER; note = 2'-O-methyladenosine-3'-phosphate 5'-phosphate 6-azidohex-1-yl ester
  modified_base 2: mod_base = OTHER; note = 2'-O-methyladenosine-3'-phosphate
  modified_base 3: mod_base = OTHER; note = 2'-deoxy-2'-fluoroadenosine-3'-phosphate
  modified_base 4: mod_base = OTHER; note = 2'-O-methyladenosine-3'-phosphate
  modified_base 5: mod_base = OTHER; note = 2'-deoxy-2'-fluorocytidine-3'-phosphate
  modified_base 6: mod_base = OTHER; note = 2'-O-methyladenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-deoxy-2'-fluoroguanosine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methylcytidine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluorouridine-3'-phosphate
  modified_base 22: mod_base = OTHER; note = 2'-O-methyladenosine-3'-phosphate
  modified_base 23: mod_base = OTHER; note = 2'-deoxy-2'-fluoroadenosine-3'-phosphate ((2S,4R)-N -(6-(1-hex-5-yn-1-onylamino)-1-hexan-1-onyl)-4-hydroxypyrr olidin-2-yl)methyl ester
aaaacagtgttcttgctctataa

SEQ ID NO: 79 - RNA, 23 nt, synthetic construct
  source 1..23: mol_type = other RNA
  modified_base 1: mod_base = OTHER; note = 2'-O-methyladenosine-3'-phosphate 5'-phosphate 6-azidohex-1-yl ester
  modified_base 2: mod_base = OTHER; note = 2'-O-methyladenosine-3'-phosphate
  modified_base 3: mod_base = OTHER; note = 2'-deoxy-2'-fluoroadenosine-3'-phosphate
  modified_base 4: mod_base = OTHER; note = 2'-O-methyladenosine-3'-phosphate
  modified_base 5: mod_base = OTHER; note = 2'-deoxy-2'-fluorocytidine-3'-phosphate
  modified_base 6: mod_base = OTHER; note = 2'-O-methyladenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-O-methyluridine-3'-phosphate
  modified_base 11: mod_base = OTHER; note = 2'-deoxy-2'-fluorouridine-3'-phosphate
  modified_base 12: mod_base = OTHER; note = 2'-deoxy-2'-fluorocytidine-3'-phosphate
  modified_base 13: mod_base = OTHER; note = 2'-deoxy-2'-fluorouridine-3'-phosphate
  modified_base 14: mod_base = OTHER; note = 2'-O-methyluridine-3'-phosphate
  modified_base 15: mod_base = OTHER; note = 2'-deoxy-2'-fluoroguanosine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methylcytidine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluorouridine-3'-phosphate
  modified_base 22: mod_base = OTHER; note = 2'-O-methyladenosine-3'-phosphate
  modified_base 23: mod_base = OTHER; note = 2'-deoxy-2'-fluoroadenosine-3'-phosphate 0
  misc_RNA 1..23: note = cyclic structure formed by trizaole-forming click cyclization of 5'-terminal azide with the 3'-terminal alkyne
aaaacagtgttcttgctctataa

SEQ ID NO: 80 - RNA, 21 nt, synthetic construct
  source 1..21: mol_type = other RNA
  modified_base 1: mod_base = OTHER; note = 2'-deoxy-2'-fluoroadenosine-3'-phosphorothioate
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-deoxy-2'-fluoroguanosine-3'-phosphate
  modified_base 14: mod_base = OTHER; note = 2'-O-methylcytidine-3'-phosphate
  modified_base 15: mod_base = OTHER; note = 2'-deoxy-2'-fluorouridine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methyladenosine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluoroadenosine-3'-phosphate ((2S,4R)-1 -[29-[[2-(acetylamino)-2-deoxy-beta-D-galactopyranosyl]oxy ]-14,14-bis[[3-[[3-[[5-[[2-(acetylamino)-2-deoxy-beta-D-ga lactopyranosyl]oxy]-1-oxopentyl]amino]propyl]amino]-3-oxop ropoxy]methyl]-1,12,19,25-tetraoxo-16-oxa-13,20,24-triazan onacos-1-yl]-4-hydroxy-2-pyrrolidinyl)methyl ester
aacagtgttcttgctctataa

SEQ ID NO: 81 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 82 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-deoxy-2'-fluorouridine-3'-phosphate
  modified_base 5: mod_base = OTHER; note = 2'-O-methyladenosine-3'-phosphate
  modified_base 6: mod_base = OTHER; note = 2'-deoxy-2'-fluoroguanosine-3'-phosphate
  modified_base 7: mod_base = OTHER; note = 2'-O-methyladenosine-3'-phosphate
  modified_base 8: mod_base = OTHER; note = 2'-deoxy-2'-fluoroguanosine-3'-phosphate
  modified_base 9: mod_base = OTHER; note = 2'-O-methylcytidine-3'-phosphate
  modified_base 10: mod_base = OTHER; note = 2'-deoxy-2'-fluoroadenosine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
ttatagagcaagaacactgtttt

SEQ ID NO: 83 - RNA, 21 nt, synthetic construct
  source 1..21: mol_type = other RNA
  modified_base 1: mod_base = OTHER; note = 2'-deoxy-2'-fluoroadenosine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-deoxy-2'-fluoroguanosine-3'-phosphate
  modified_base 14: mod_base = OTHER; note = 2'-O-methylcytidine-3'-phosphate
  modified_base 15: mod_base = OTHER; note = 2'-deoxy-2'-fluorouridine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methyladenosine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluoroadenosine-3'-phosphate (4R-((hydr oxy(N-[29-[[2-(acetylamino)-2-deoxy-beta-D-galactopyranosy l]oxy]-14,14-bis[[3-[[3-[[5-[[2-(acetylamino)-2-deoxy-beta -D-galactopyranosyl]oxy]-1-oxopentyl]amino]propyl]amino]-3 -oxopropoxy]methyl]-1,12,19,25-tetraoxo-16-oxa-13,20,24-tr iazanonacos-1-yl]-4R-hydroxypyrrolidin-2S-ylmethoxy)phosph oryl)oxy)-N-(6-(1-hex-5-yn-1-onylamino)-1-hexan-1-onyl)-py rrolidin-2S-yl)methyl ester
aacagtgttcttgctctataa

SEQ ID NO: 84 - RNA, 21 nt, synthetic construct
  source 1..21: mol_type = other RNA
  modified_base 1: mod_base = OTHER; note = 2'-deoxy-2'-fluoroadenosine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-deoxy-2'-fluoroguanosine-3'-phosphate
  modified_base 14: mod_base = OTHER; note = 2'-O-methylcytidine-3'-phosphate
  modified_base 15: mod_base = OTHER; note = 2'-deoxy-2'-fluorouridine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methyladenosine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluoroadenosine-3'-phosphate (4R-((hydr oxy(N-[29-[[2-(acetylamino)-2-deoxy-beta-D-galactopyranosy l]oxy]-14,14-bis[[3-[[3-[[5-[[2-(acetylamino)-2-deoxy-beta -D-galactopyranosyl]oxy]-1-oxopentyl]amino]propyl]amino]-3 -oxopropoxy]methyl]-1,12,19,25-tetraoxo-16-oxa-13,20,24-tr iazanonacos-1-yl]-4R-hydroxypyrrolidin-2S-ylmethoxy)phosph oryl)oxy)-N-(6-(1-hex-5-yn-1-onylamino)-1-hexan-1-onyl)-py rrolidin-2S-yl)methyl ester
  misc_RNA 1..21: note = cyclic structure formed by trizaole-forming click cyclization of 5'-terminal azide with the 3'-terminal alkyne
aacagtgttcttgctctataa

SEQ ID NO: 85 - RNA, 21 nt, synthetic construct
  source 1..21: mol_type = other RNA
  modified_base 1: mod_base = OTHER; note = 2'-deoxy-2'-fluoroguanosine-3'-phosphorothioate
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroadenosine-3'-phosphate
  modified_base 6: mod_base = OTHER; note = 2'-O-methyladenosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-deoxy-2'-fluorocytidine-3'-phosphate
  modified_base 14: mod_base = OTHER; note = 2'-O-methyladenosine-3'-phosphate
  modified_base 15: mod_base = OTHER; note = 2'-deoxy-2'-fluorocytidine-3'-phosphate
  modified_base 16: mod_base = OTHER; note = 2'-O-methyluridine-3'-phosphate
  modified_base 17: mod_base = OTHER; note = 2'-deoxy-2'-fluoroadenosine-3'-phosphate
  modified_base 18: mod_base = OTHER; note = 2'-O-methyluridine-3'-phosphate
  modified_base 19: mod_base = OTHER; note = 2'-deoxy-2'-fluoroadenosine-3'-phosphate
  modified_base 20: mod_base = OTHER; note = 2'-O-methyladenosine-3'-phosphate
  modified_base 21: mod_base = OTHER; note = 2'-deoxy-2'-fluorouridine-3'-phosphate ((2S,4R)-1-[ 29-[[2-(acetylamino)-2-deoxy-beta-D-galactopyranosyl]oxy]- 14,14-bis[[3-[[3-[[5-[[2-(acetylamino)-2-deoxy-beta-D-gala ctopyranosyl]oxy]-1-oxopentyl]amino]propyl]amino]-3-oxopro poxy]methyl]-1,12,19,25-tetraoxo-16-oxa-13,20,24-triazanon acos-1-yl]-4-hydroxy-2-pyrrolidinyl)methyl ester
gacaaaataactcactataat

SEQ ID NO: 86 - RNA, 23 nt, synthetic construct
  source 1..23: mol_type = other RNA
  modified_base 1: mod_base = OTHER; note = 2'-O-methyladenosine-3'-phosphorothioate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-deoxy-2'-fluoroadenosine-3'-phosphate
  modified_base 5: mod_base = OTHER; note = 2'-O-methyluridine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-O-methylguanosine-3'-phosphate
  modified_base 10: mod_base = OTHER; note = 2'-deoxy-2'-fluoroadenosine-3'-phosphate
  modified_base 11: mod_base = OTHER; note = 2'-O-methylguanosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorouridine-3'-phosphate
  modified_base 17: mod_base = OTHER; note = 2'-O-methyluridine-3'-phosphate
  modified_base 18: mod_base = OTHER; note = 2'-deoxy-2'-fluorouridine-3'-phosphate
  modified_base 19: mod_base = OTHER; note = 2'-O-methylguanosine-3'-phosphate
  modified_base 20: mod_base = OTHER; note = 2'-deoxy-2'-fluorouridine-3'-phosphate
  modified_base 21: mod_base = OTHER; note = 2'-O-methylcytidine-3'-phosphorothioate
  modified_base 22: mod_base = OTHER; note = 2'-O-methyladenosine-3'-phosphorothioate
  modified_base 23: mod_base = OTHER; note = 2'-O-methyladenosine
attatagtgagttattttgtcaa

SEQ ID NO: 87 - RNA, 21 nt, synthetic construct
  source 1..21: mol_type = other RNA
  modified_base 1: mod_base = OTHER; note = 2'-deoxy-2'-fluoroguanosine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroadenosine-3'-phosphate
  modified_base 6: mod_base = OTHER; note = 2'-O-methyladenosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-deoxy-2'-fluorocytidine-3'-phosphate
  modified_base 14: mod_base = OTHER; note = 2'-O-methyladenosine-3'-phosphate
  modified_base 15: mod_base = OTHER; note = 2'-deoxy-2'-fluorocytidine-3'-phosphate
  modified_base 16: mod_base = OTHER; note = 2'-O-methyluridine-3'-phosphate
  modified_base 17: mod_base = OTHER; note = 2'-deoxy-2'-fluoroadenosine-3'-phosphate
  modified_base 18: mod_base = OTHER; note = 2'-O-methyluridine-3'-phosphate
  modified_base 19: mod_base = OTHER; note = 2'-deoxy-2'-fluoroadenosine-3'-phosphate
  modified_base 20: mod_base = OTHER; note = 2'-O-methyladenosine-3'-phosphate
  modified_base 21: mod_base = OTHER; note = 2'-deoxy-2'-fluorouridine-3'-phosphate (4R-((hydrox y(N-[29-[[2-(acetylamino)-2-deoxy-beta-D-galactopyranosyl] oxy]-14,14-bis[[3-[[3-[[5-[[2-(acetylamino)-2-deoxy-beta-D -galactopyranosyl]oxy]-1-oxopentyl]amino]propyl]amino]-3-o xopropoxy]methyl]-1,12,19,25-tetraoxo-16-oxa-13,20,24-tria zanonacos-1-yl]-4R-hydroxypyrrolidin-2S-ylmethoxy)phosphor yl)oxy)-N-(6-(1-hex-5-yn-1-onylamino)-1-hexan-1-onyl)-pyrr olidin-2S-yl)methyl ester
  misc_RNA 1..21: note = cyclic structure formed by trizaole-forming click cyclization of 5'-terminal azide with the 3'-terminal alkyne
gacaaaataactcactataat

SEQ ID NO: 88 - RNA, 20 nt, synthetic construct
  source 1..20: mol_type = other RNA
  modified_base 2: mod_base = OTHER; note = 2'-O-methyladenosine-3'-phosphate
  modified_base 4: mod_base = OTHER; note = 2'-O-methyladenosine-3'-phosphate
  modified_base 6: mod_base = OTHER; note = 2'-O-methylcytidine-3'-phosphate
  modified_base 8: mod_base = OTHER; note = 2'-O-methyladenosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 12: mod_base = OTHER; note = 2'-O-methylcytidine-3'-phosphate
  modified_base 14: mod_base = OTHER; note = 2'-O-methylcytidine-3'-phosphate
  modified_base 16: mod_base = OTHER; note = 2'-O-methylguanosine-3'-phosphate
  modified_base 18: mod_base = OTHER; note = 2'-O-methyluridine-3'-phosphorothioate
  modified_base 19: mod_base = OTHER; note = 2'-O-methyluridine-3'-phosphorothioate
  modified_base 20: mod_base = OTHER; note = 2'-O-methyluridine
tagagcaagaacactgtttt

SEQ ID NO: 89 - RNA, 19 nt, synthetic construct
  source 1..19: mol_type = other RNA
  modified_base 1: mod_base = OTHER; note = 2'-O-methyluridine-3'-phosphorothioate 5'phosphate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine-3'-phosphate
  modified_base 19: mod_base = OTHER; note = 2'-O-methylguanosine
ttatagagcaagaacactg

SEQ ID NO: 90 - RNA, 19 nt, synthetic construct
  source 1..19: mol_type = other RNA
  modified_base 1: mod_base = OTHER; note = uridine-3'-phosphorothioate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine-3'-phosphate
  modified_base 19: mod_base = OTHER; note = 2'-O-methylguanosine
ttatagagcaagaacactg

SEQ ID NO: 91 - RNA, 17 nt, synthetic construct
  source 1..17: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine
ttatagagcaagaacac

SEQ ID NO: 92 - RNA, 21 nt, synthetic construct
  source 1..21: mol_type = other RNA
  modified_base 1: mod_base = OTHER; note = 2'-deoxy-2'-fluoroadenosine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-deoxy-2'-fluorocytidine-3'-phosphate
  modified_base 4: mod_base = OTHER; note = 2'-O-methyladenosine-3'-phosphate
  modified_base 5: mod_base = OTHER; note = 2'-deoxy-2'-fluoroguanosine-3'-phosphate
  modified_base 6: mod_base = OTHER; note = 2'-O-methyluridine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-deoxy-2'-fluoroguanosine-3'-phosphate
  modified_base 14: mod_base = OTHER; note = 2'-O-methylcytidine-3'-phosphate
  modified_base 15: mod_base = OTHER; note = 2'-deoxy-2'-fluorouridine-3'-phosphate
  modified_base 16: mod_base = OTHER; note = 2'-O-methylcytidine-3'-phosphate
  modified_base 17: mod_base = OTHER; note = 2'-deoxy-2'-fluorouridine-3'-phosphate
  modified_base 18: mod_base = OTHER; note = 2'-O-methyladenosine-3'-phosphate
  modified_base 19: mod_base = OTHER; note = 2'-deoxy-2'-fluorouridine-3'-phosphate
  modified_base 20: mod_base = OTHER; note = 2'-O-methyladenosine-3'-phosphate
  modified_base 21: mod_base = OTHER; note = 2'-deoxy-2'-fluoroadenosine-3'-phosphate (4R-((hydr oxy(N-[29-[[2-(acetylamino)-2-deoxy-beta-D-galactopyranosy l]oxy]-14,14-bis[[3-[[3-[[5-[[2-(acetylamino)-2-deoxy-beta -D-galactopyranosyl]oxy]-1-oxopentyl]amino]propyl]amino]-3 -oxopropoxy]methyl]-1,12,19,25-tetraoxo-16-oxa-13,20,24-tr iazanonacos-1-yl]-4R-hydroxypyrrolidin-2S-ylmethoxy)phosph oryl)oxy)-N-(6-(1-hex-5-yn-1-onylamino)-1-hexan-1-onyl)-py rrolidin-2S-yl)methyl ester, with the loss of single GalNAc sugar from the triantenary ligand
aacagtgttcttgctctataa

SEQ ID NO: 93 - RNA, 20 nt, synthetic construct
  source 1..20: mol_type = other RNA
  modified_base 2: mod_base = OTHER; note = 2'-O-methyladenosine-3'-phosphate
  modified_base 4: mod_base = OTHER; note = 2'-O-methyladenosine-3'-phosphate
  modified_base 6: mod_base = OTHER; note = 2'-O-methylcytidine-3'-phosphate
  modified_base 8: mod_base = OTHER; note = 2'-O-methyladenosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 12: mod_base = OTHER; note = 2'-O-methylcytidine-3'-phosphate
  modified_base 14: mod_base = OTHER; note = 2'-O-methylcytidine-3'-phosphate
  modified_base 16: mod_base = OTHER; note = 2'-O-methylguanosine-3'-phosphate
  modified_base 18: mod_base = OTHER; note = 2'-O-methyluridine-3'-phosphorothioate
  modified_base 19: mod_base = OTHER; note = 2'-O-methyluridine-3'-phosphorothioate
  modified_base 20: mod_base = OTHER; note = 2'-O-methyluridine
tagagcaagaacactgtttt

SEQ ID NO: 94 - RNA, 19 nt, synthetic construct
  source 1..19: mol_type = other RNA
  modified_base 1: mod_base = OTHER; note = uridine-3'-phosphorothioate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine-3'-phosphate
  modified_base 19: mod_base = OTHER; note = 2'-O-methylguanosine
ttatagagcaagaacactg

SEQ ID NO: 95 - skipped

SEQ ID NO: 96 - RNA, 19 nt, synthetic construct
  source 1..19: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine-3'-phosphate
  modified_base 19: mod_base = OTHER; note = 2'-O-methylguanosine-3'-phosphate
ttatagagcaagaacactg

SEQ ID NO: 97 - RNA, 19 nt, synthetic construct
  source 1..19: mol_type = other RNA
  modified_base 1: mod_base = OTHER; note = 2'-O-methyluridine-3'-phosphorothioate 5'phosphate
  modified_base 2: mod_base = OTHER; note = uridine-3'-phosphorothioate
  modified_base 3: mod_base = OTHER; note = 2'-O-methyladenosine-3'-phosphate
  modified_base 5: mod_base = OTHER; note = 2'-O-methyladenosine-3'-phosphate
  modified_base 7: mod_base = OTHER; note = 2'-O-methyladenosine-3'-phosphate
  modified_base 9: mod_base = OTHER; note = 2'-O-methylcytidine-3'-phosphate
  modified_base 11: mod_base = OTHER; note = 2'-O-methyladenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 15: mod_base = OTHER; note = 2'-O-methylcytidine-3'-phosphate
  modified_base 17: mod_base = OTHER; note = 2'-O-methylcytidine-3'-phosphate
  modified_base 19: mod_base = OTHER; note = 2'-O-methylguanosine
ttatagagcaagaacactg

SEQ ID NO: 98 - RNA, 21 nt, synthetic construct
  source 1..21: mol_type = other RNA
  modified_base 1: mod_base = OTHER; note = adenosine-3'-phosphorothioate 5'-phosphate 6-azidohex-1-yl ester
  modified_base 2: mod_base = OTHER; note = 2'-O-methyladenosine-3'-phosphorothioate
  modified_base 4: mod_base = OTHER; note = 2'-O-methyladenosine-3'-phosphate
  modified_base 6: mod_base = OTHER; note = 2'-O-methyluridine-3'-phosphate
  modified_base 8: mod_base = OTHER; note = 2'-O-methyluridine-3'-phosphate
  modified_base 12: mod_base = OTHER; note = 2'-O-methyluridine-3'-phosphate
  modified_base 14: mod_base = OTHER; note = 2'-O-methylcytidine-3'-phosphate
  modified_base 16: mod_base = OTHER; note = 2'-O-methylcytidine-3'-phosphate
  modified_base 18: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = adenosine-3'-phosphate (4R-((hydroxy(N-[29-[[2-(ace tylamino)-2-deoxy-beta-D-galactopyranosyl]oxy]-14,14-bis[[ 3-[[3-[[5-[[2-(acetylamino)-2-deoxy-beta-D-galactopyranosy l]oxy]-1-oxopentyl]amino]propyl]amino]-3-oxopropoxy]methyl ]-1,12,19,25-tetraoxo-16-oxa-13,20,24-triazanonacos-1-yl]- 4R-hydroxypyrrolidin-2S-ylmethoxy)phosphoryl)oxy)-N-(6-(1- hex-5-yn-1-onylamino)-1-hexan-1-onyl)-pyrrolidin-2S-yl)met hyl ester
aacagtgttcttgctctataa

SEQ ID NO: 99 - RNA, 21 nt, synthetic construct
  source 1..21: mol_type = other RNA
  modified_base 1: mod_base = OTHER; note = adenosine-3'-phosphorothioate 5'-phosphate 6-azidohex-1-yl ester
  modified_base 2: mod_base = OTHER; note = 2'-O-methyladenosine-3'-phosphorothioate
  modified_base 4: mod_base = OTHER; note = 2'-O-methyladenosine-3'-phosphate
  modified_base 6: mod_base = OTHER; note = 2'-O-methyluridine-3'-phosphate
  modified_base 8: mod_base = OTHER; note = 2'-O-methyluridine-3'-phosphate
  modified_base 12: mod_base = OTHER; note = 2'-O-methyluridine-3'-phosphate
  modified_base 14: mod_base = OTHER; note = 2'-O-methylcytidine-3'-phosphate
  modified_base 16: mod_base = OTHER; note = 2'-O-methylcytidine-3'-phosphate
  modified_base 18: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = adenosine-3'-phosphate (4R-((hydroxy(N-[29-[[2-(ace tylamino)-2-deoxy-beta-D-galactopyranosyl]oxy]-14,14-bis[[ 3-[[3-[[5-[[2-(acetylamino)-2-deoxy-beta-D-galactopyranosy l]oxy]-1-oxopentyl]amino]propyl]amino]-3-oxopropoxy]methyl ]-1,12,19,25-tetraoxo-16-oxa-13,20,24-triazanonacos-1-yl]- 4R-hydroxypyrrolidin-2S-ylmethoxy)phosphoryl)oxy)-N-(6-(1- hex-5-yn-1-onylamino)-1-hexan-1-onyl)-pyrrolidin-2S-yl)met hyl ester
  misc_RNA 1..21: note = cyclic structure formed by trizaole-forming click cyclization of 5'-terminal azide with the 3'-terminal alkyne
aacagtgttcttgctctataa

SEQ ID NO: 100 - skipped

SEQ ID NO: 101 - RNA, 21 nt, synthetic construct
  source 1..21: mol_type = other RNA
  modified_base 1: mod_base = OTHER; note = adenosine-3'-phosphorothioate 5'-phosphate 6-azidohex-1-yl ester
  modified_base 2: mod_base = OTHER; note = 2'-O-methyladenosine-3'-phosphorothioate
  modified_base 4: mod_base = OTHER; note = 2'-O-methyladenosine-3'-phosphate
  modified_base 6: mod_base = OTHER; note = 2'-O-methyluridine-3'-phosphate
  modified_base 8: mod_base = OTHER; note = 2'-O-methyluridine-3'-phosphate
  modified_base 12: mod_base = OTHER; note = 2'-O-methyluridine-3'-phosphate
  modified_base 14: mod_base = OTHER; note = 2'-O-methylcytidine-3'-phosphate
  modified_base 16: mod_base = OTHER; note = 2'-O-methylcytidine-3'-phosphate
  modified_base 18: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = adenosine-3'-phosphate (4R-((hydroxy(N-[29-[[2-(ace tylamino)-2-deoxy-beta-D-galactopyranosyl]oxy]-14,14-bis[[ 3-[[3-[[5-[[2-(acetylamino)-2-deoxy-beta-D-galactopyranosy l]oxy]-1-oxopentyl]amino]propyl]amino]-3-oxopropoxy]methyl ]-1,12,19,25-tetraoxo-16-oxa-13,20,24-triazanonacos-1-yl]- 4R-hydroxypyrrolidin-2S-ylmethoxy)phosphoryl)oxy)-N-(6-(1- hex-5-yn-1-onylamino)-1-hexan-1-onyl)-pyrrolidin-2S-yl)met hyl ester, with the loss of single GalNAc sugar from the triantenary ligand
aacagtgttcttgctctataa

SEQ ID NO: 102 - RNA, 45 nt, synthetic construct; identical to SEQ ID NO: 5
  source 1..45: mol_type = other RNA
  modified_base 1: mod_base = OTHER; note = 2'-O-methyluridine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-O-methyluridine-3'-phosphate
  modified_base 23: mod_base = OTHER; note = 2'-O-methyluridine-3'-phosphate
  modified_base 24: mod_base = OTHER; note = 2'-O-methyluridine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-methyluridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-prop-2-yn-1-yl-adenosine
  misc_RNA 1..45: note = cyclic structure formed by trizaole-forming click cyclization of 5'-terminal azide with the 3'-terminal alkyne

SEQ ID NO: 103 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluorocytidine-3'-phosphate
  modified_base 15: mod_base = OTHER; note = 2'-O-methyladenosine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
tttcgaatcaatccaacagtagc

SEQ ID NO: 104 - RNA, 45 nt, synthetic construct; identical to SEQ ID NO: 5
  source 1..45: mol_type = other RNA
  modified_base 1: mod_base = OTHER; note = 2'-O-methyluridine-3'-phosphorothioate 5'-phosphate 6-azidohex-1-yl ester
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = 2'-deoxy-2'-fluorouridine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorouridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  modified_base 22: mod_base = OTHER; note = 2'-O-methyluridine-3'-phosphate
  modified_base 23: mod_base = OTHER; note = 2'-O-methyluridine-3'-phosphate
  modified_base 24: mod_base = OTHER; note = 2'-O-methyluridine-3'-phosphate
  modified_base 25: mod_base = OTHER; note = 2'-O-methylcytidine-3'-phosphorothioate
  modified_base 26: mod_base = OTHER; note = 2'-O-methyladenosine-3'-phosphorothioate
  modified_base 27: mod_base = OTHER; note = 2'-O-methyluridine-3'-phosphate
  modified_base 28: mod_base = OTHER; note = 2'-O-methyluridine-3'-phosphate
  modified_base 29: mod_base = OTHER; note = 2'-O-methyluridine-3'-phosphate
  modified_base 30: mod_base = OTHER; note = 2'-O-methyluridine-3'-phosphate
  modified_base 31: mod_base = OTHER; note = 2'-deoxy-2'-fluoroadenosine-3'-phosphate
  modified_base 32: mod_base = OTHER; note = 2'-O-methyladenosine-3'-phosphate
  modified_base 33: mod_base = OTHER; note = 2'-deoxy-2'-fluorouridine-3'-phosphate
  modified_base 34: mod_base = OTHER; note = 2'-deoxy-2'-fluorocytidine-3'-phosphate
  modified_base 35: mod_base = OTHER; note = 2'-deoxy-2'-fluorocytidine-3'-phosphate
  modified_base 36: mod_base = OTHER; note = 2'-O-methyluridine-3'-phosphate
  modified_base 37: mod_base = OTHER; note = 2'-O-methylcytidine-3'-phosphate
  modified_base 38: mod_base = OTHER; note = 2'-O-methyladenosine-3'-phosphate
  modified_base 39: mod_base = OTHER; note = 2'-O-methylcytidine-3'-phosphate
  modified_base 40: mod_base = OTHER; note = 2'-O-methyluridine-3'-phosphate
  modified_base 41: mod_base = OTHER; note = 2'-O-methylcytidine-3'-phosphate
  modified_base 42: mod_base = OTHER; note = 2'-O-methyluridine-3'-phosphate
  modified_base 43: mod_base = OTHER; note = 2'-O-methyladenosine-3'-phosphorothioate
  modified_base 44: mod_base = OTHER; note = 2'-O-methyladenosine-3'-phosphorothioate
  modified_base 45: mod_base = OTHER; note = 2'-O-prop-2-yn-1-yl-adenosine-3'-phosphate ((2S,4R) -1-[29-[[2-(acetylamino)-2-deoxy-beta-D-galactopyranosyl]o xy]-14,14-bis[[3-[[3-[[5-[[2-(acetylamino)-2-deoxy-beta-D- galactopyranosyl]oxy]-1-oxopentyl]amino]propyl]amino]-3-ox opropoxy]methyl]-1,12,19,25-tetraoxo-16-oxa-13,20,24-triaz anonacos-1-yl]-4-hydroxy-2-pyrrolidinyl)methyl ester
  misc_RNA 1..45: note = cyclic structure formed by trizaole-forming click cyclization of 5'-terminal azide with the 3'-terminal alkyne

SEQ ID NO: 105 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorocytidine-3'-phosphate
  modified_base 10: mod_base = OTHER; note = 2'-O-methyladenosine-3'-phosphate
  modified_base 11: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluorocytidine-3'-phosphate
  modified_base 15: mod_base = OTHER; note = 2'-O-methyladenosine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
tttcgaatcaatccaacagtagc

SEQ ID NO: 106 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

SEQ ID NO: 107 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
tttagagtgaggattaaaatgag

(19) United States
(12) Patent Application Publication
(10) Pub. No.: US 2025/0270562 A1
(43) Pub. Date: Aug. 28, 2025

(54) BIS-RNAI COMPOUNDS FOR CNS DELIVERY

(71) Applicant: ALNYLAM PHARMACEUTICALS, INC., Cambridge, MA (US)

(72) Inventors: Ivan ZLATEV, Cambridge, MA (US); Christopher S. THEILE, Cambridge, MA (US); Muthiah MANOHARAN, Cambridge, MA (US); Scott P. LENTINI, Cambridge, MA (US); Shigeo MATSUDA, Cambridge, MA (US); Jayaprakash K. NAIR, Cambridge, MA (US); Haiyan PENG, Cambridge, MA (US)

(21) Appl. No.: 18/577,377

(22) PCT Filed: Jul. 8, 2022
(86) PCT No.: PCT/US2022/036455

§ 371 Date: Jan. 8, 2024

(51) Int. Cl. C12N 15/113

(52) U.S. Cl. CPC C12N 15/1137; C12N 15/113; C12N 2310/14; C12N 2310/321; C12N 2310/351; C12N 2310/532

(57) ABSTRACT

One aspect of the invention relates to molecules that target more than one target nucleic acid sequence and that exhibit efficacy in the tissues of the CNS of a subject upon contact. Another aspect of the present invention relates to a small circular interfering RNA (sciRNA) for modulating one or more target mRNAs in the central nervous system (CNS) of a subject. Other aspects of the invention relate a pharmaceutical composition, a method for inhibiting the expression of one or more target mRNAs in the CNS of a subject, and a method of treating or preventing a CNS disease or disorder in a subject using the molecules and sciRNA.

### CROSS-REFERENCE TO RELATED APPLICATIONS

This application claims benefit of priority to U.S. Provisional Application No. 63/219,930, filed Jul. 9, 2021, and U.S. Provisional Application No. 63/220,232 filed Jul. 9, 2021, both of which are herein incorporated by reference in their entirety.

### FIELD OF INVENTION

This invention generally relates to the field of RNA interference with bis-RNAi compounds, useful for modulating gene expression of multiple targets, particularly in central nervous system (CNS) cells and tissues.

### BACKGROUND

Chemical modifications of the nucleobases, ribose sugar, and phosphate backbone have been used to improve drug-like properties of therapeutic oligonucleotides and to confer favorable pharmacological properties to GalNAc-siRNA conjugates in preclinical and clinical development.

Nevertheless, relatively few alterations have been performed at the level of the three-dimensional structure of siRNAs. Limited examples of supra-RNAi structures have been reported, including hairpin siRNAs (Yu et al., “RNA interference by expression of short-interfering RNAs and hairpin RNAs in mammalian cells,” 99: 6047-52 (2002)), dumbbell-shaped nanocircular siRNAs (Abe et al., “Dumbbell-shaped nanocircular RNAs for RNA interference,” J. Am. Chem. Soc., 129: 15108-09 (2007)), siRNA nanosheets (Kim et al., “Generation of siRNA Nanosheets for Efficient RNA Interference,” Sci. Rep., 6: 25146 (2016)), branched siRNAs (Avino et al., “Branched RNA: A new architecture for RNA interference,” J. Nucleic Acids, 2011: 586935 (2011)), caged circular siRNAs for photomodulation of gene expression (Zhang et al., “Caged circular siRNAs for photomodulation of gene expression in cells and mice,” Chem. Sci., 9: 44-51 (2018)), circular single strand RNAs as siRNA precursors (Kimura et al., “Intracellular build-up RNAi with single-strand circular RNAs as siRNA precursors,” Chem. Commun., 10.1039/C1039CC04872C (2019)), and circular siRNAs with reduced off-target effects (Abe et al., “Synthesis and characterization of small circular double-stranded RNAs,” Chem. Commun., 47: 2125-2127 (2011); Zhang et al., “Circular siRNAs for reducing off-target effects and enhancing long-term gene silencing in cells and mice,” Mol. Ther. —Nucleic Acids, 10: 237-44 (2018)).

However, these reports were based on natural ribonucleotides and phosphodiester linkages, did not utilize therapeutically relevant siRNA chemical modifications, employed inefficient cyclization strategies, such as peptide coupling or T4 ligation techniques, and/or only achieved modest yields. Moreover, some of these reports indicated that cyclizing the antisense (guide) strands into circular siRNAs have resulted in the loss of silencing activity (see e.g., Zhang et al., “Caged circular siRNAs for photomodulation of gene expression in cells and mice,” Chem. Sci., 9: 44-51 (2018)), which may be due to the inability of a circular antisense strand to get loaded onto the Argonaute 2 (Ago2) protein, precluding the formation of an active RNA-induced silencing complex (RISC), the driving component of RNA interference-mediated mRNA silencing. Some of these reports also discouraged chemical modifications, such as modifications with 2′OMe RNA, locked nucleic acid, unlocked nucleoside analogs, 5-nitroindole-modified nucleotide, terminal methylation, backbone phosphothioate, etc., suggesting that these modifications may prevent the loading and processing of sense strand RNA to lower the off-target effect of siRNAs (see e.g., Zhang et al., “Circular siRNAs for reducing off-target effects and enhancing long-term gene silencing in cells and mice,” Mol. Ther. —Nucleic Acids, 10: 237-44 (2018)). In the reports describing an in vivo application, no systemic administration or targeting of exogenous gene expression were described, nor was a therapeutically relevant delivery reagent used for this locally administered high dose siRNA.

Thus, there is a continuing need for a new and improved design for three-dimensional siRNA duplex structure to achieve and enhance the therapeutic potential of RNAi agents, such as enhancing their potency, metabolic stability, and off-target properties, particularly for molecules that can modulate gene expression of multiple target nucleic acids, while achieving efficient delivery and efficacy in one or more tissues, especially in central nervous system (CNS) cells and tissues. There is also a need in the art for molecules that can target more than one target nucleic acid, while achieving efficient delivery and efficacy in one or more tissues of the central nervous system (CNS) of a subject.

### SUMMARY

The present disclosure provides molecules designed to target more than one target nucleic acid, or the same target nucleic acid two or more times within the same agent, or two or more distinct target RNA sequences within one or more target nucleic acids, and that exhibit delivery to and surprising efficacy in a CNS tissue of a subject upon contact. Pharmaceutical compositions, methods, and other related aspects are also provided.

One aspect of the invention provides a nucleic acid composition for modulating in the central nervous system (CNS) of a subject one or more target RNAs comprising one or more distinct target RNA sequences, the nucleic acid composition having a first double-stranded RNA (dsRNA) molecule and a single-stranded nucleic acid agent or a second dsRNA molecule, wherein the first dsRNA molecule and the single-stranded nucleic acid agent or second dsRNA molecule are connected together by a linker and do not overlap with each other, the first dsRNA includes at least one conjugated lipophilic moiety, the second dsRNA molecule, if present, includes at least one conjugated lipophilic moiety, and each of the first dsRNA molecule and the single-stranded nucleic acid agent or second dsRNA molecule of the nucleic acid composition is capable of modulating the activity or expression of the one or more target RNAs in a tissue of the CNS of the subject by at least 15% relative to an appropriate control.

In certain embodiments, the first dsRNA molecule and the second dsRNA molecule are connected together by the linker.

In certain embodiments, the first dsRNA molecule and the single-stranded nucleic acid agent connected together by the linker.

In certain embodiments, the single-stranded nucleic acid agent is an inhibitory single-stranded oligonucleotide or a single-stranded small interfering RNA (ss-siRNA).

In certain embodiments, the nucleic acid composition is capable of inhibiting the activity or expression of the one or more target RNAs in a tissue of the CNS of the subject. Optionally, the nucleic acid composition inhibits the activity or expression of the one or more distinct target RNAs in a tissue of the CNS of the subject.

In some embodiments, the nucleic acid composition is capable of inhibiting the activity or expression of two or more target RNAs in a tissue of the CNS of the subject. Optionally, the nucleic acid composition inhibits the activity or expression of two or more distinct target RNAs in a tissue of the CNS of the subject.

In certain embodiments, the single-stranded nucleic acid agent includes at least one conjugated lipophilic moiety.

In some embodiments, the multi-targeted molecule does not modulate gene expression by two different mechanisms.

In certain embodiments, each nucleic acid-based effector molecule in the multi-targeted molecule can modulate gene expression of a target nucleic acid. Without limitations, each effector molecule in the multi-targeted molecule can be directed to the same target gene, different target genes, different positions within the same target gene, or different transcripts of the same target gene. Further, it is noted that said effector molecules included in the multi-targeted molecules disclosed herein can include any of the nucleic acid modifications, motifs or structures described herein or otherwise known in the art.

Moreover, the effector molecules included in the multi-targeted molecules described herein have comparable gene expression modulating activity compared to the gene expression modulating activity when said effector molecules are not part of a multi-targeted molecule. In other words, an effector molecule has similar gene expression modulating activity when it is part of a multi-targeted molecule disclosed herein relative to when it is not part of a multi-targeted molecule. In some embodiments, the effector molecules included in the multi-targeted molecule described herein can independently modulate gene expression of their respective target nucleic acids by at least 15%, optionally at least 20%, optionally at least 25%, optionally at least 30%, at least 35%, at least 40%, at least 45%, or at least 50% (e.g., 50%, 60%, 70%, 75%, 80%, 85%, 90%, 95% or more) relative to their modulation of gene expression when not part of a multi-targeted molecule. In some embodiments, one of the effector molecules in the multi-targeted molecule modulates gene expression at a higher level relative to the other effector molecule in said multi-targeted molecule. In some embodiments, said at least two effector molecules in a multi-targeted molecule modulate gene expression at similar levels (e.g., within 10%, 7.5%, 5%, 2.5% or less of each other).

In some embodiments, each effector molecule of a multi-targeted molecule of the instant disclosure is capable of inhibiting expression of a target mRNA by at least 15% in the CNS of a subject, as compared to an appropriate control (e.g., as compared to an untreated or placebo-treated subject, or as compared to a reference value, including, e.g., target mRNA or protein levels in the treated subject measured before treatment with the multi-targeted molecule occurred). In related embodiments, a multi-targeted molecule of the instant disclosure is capable of inhibiting expression of a target mRNA by at least 20%, optionally by at least 25%, optionally by at least 30%, optionally by at least 35%, optionally by at least 40%, optionally by at least 45%, optionally by at least 50%, optionally by at least 55%, optionally by at least 60%, optionally by at least 65%, optionally by at least 70%, optionally by at least 75%, optionally by at least 80%, optionally by at least 85%, optionally by at least 90%, optionally by at least 95% in the CNS of a subject, as compared to an appropriate control.

In some embodiments, the multi-targeted molecule modulates gene expression of at least two target nucleic acids by at least 75% each relative to when the effector molecules are not connected together.

In some embodiments, one of the at least two effector molecules modulates gene expression of a first target nucleic acid and another one of the at least two effector molecules modulates gene expression of a second nucleic acid. In certain embodiments, the first target nucleic acid and the second target nucleic are located in different transcripts, or genes from each other. In some embodiments, the first target nucleic acid and the second target nucleic are located in the same nucleic acid.

In related embodiments, the multi-targeted molecule is capable of inhibiting expression of a target mRNA throughout the CNS of a subject, or within a location within the CNS of a subject. In certain embodiments, the multi-targeted molecule is capable of inhibiting expression of a target mRNA in one or more of the following CNS locations of a subject: right hemisphere, left hemisphere, cerebellum, striatum, brainstem, and spinal cord. In some embodiments, CNS cell types are targeted, including neurons, oligodendrocytes, microglia, and astrocytes, among others.

It has been discovered herein that multi-targeted molecules conjugated with at least one lipophilic ligand on each effector molecule/component are particularly effective in modulating gene expression. Accordingly, in some embodiments, at least two lipophilic ligands are conjugated with the multi-targeted molecule (in a distribution that positions at least one lipophilic ligand upon each effector molecule/component). The two ligands can be conjugated at independently at any position in the multi-targeted molecule, provided that each effector molecule/component carries a lipophilic ligand. In such embodiments, at least two effector molecules in the multi-targeted molecule have at least one lipophilic ligand attached thereto. As such, multi-targeted molecules conjugated with at least two lipophilic ligands are also referred to as “conjugated multi-targeted molecule” herein. Without limitation, each ligand can be present at any position of the effector molecule and/or the multi-targeted molecule. For example, each ligand can be conjugated at the 5′-end, 3′-end an internal (non-terminal) position of an effector molecule, or combinations thereof in the multi-targeted molecule. The said at least two ligands can be the same, different or any combinations of same and different. Without wishing to be bound by theory, inclusion of at least one lipophilic ligand upon each effector molecule/component was surprisingly identified to improve delivery or pharmacokinetic profile of the conjugated multi-targeted molecule when administered to the CNS of a subject, whereas neither effector molecule was identified to be an effective agent in tested multi-targeted molecules harboring only a single lipophilic ligand.

In some embodiments, provided herein is a multi-targeted molecule for modulation of two or more distinct target RNAs in the central nervous system (CNS) of a subject, the multi-targeted molecule having a first double-stranded RNA (dsRNA) molecule and a second double-stranded RNA molecule, where: the first dsRNA and second dsRNA molecules are connected together by a linker and do not overlap with each other, each of the first dsRNA and second dsRNA includes at least one conjugated lipophilic moiety, and the multi-targeted molecule is capable of inhibiting the activity or expression of the two or more distinct target RNAs in a tissue of the CNS of the subject by at least 15% each, relative to an appropriate control.

In certain embodiments, the lipophilicity of each lipophilic moiety, measured by log K_{ow}, exceeds 0.

In one embodiment, the hydrophobicity of the multi-targeted molecule, measured by the unbound fraction in a plasma protein binding assay of the multi-targeted molecule, exceeds 0.2.

In some embodiments, each lipophilic moiety is one or more of a lipid, a cholesterol, a retinoic acid, a cholic acid, an adamantane acetic acid, a 1-pyrene butyric acid, a dihydrotestosterone, a 1,3-bis-O(hexadecyl)glycerol, a geranyloxyhexyanol, a hexadecylglycerol, a borneol, a menthol, a 1,3-propanediol, a heptadecyl group, a palmitic acid, a myristic acid, an O3-(oleoyl)lithocholic acid, an O3-(oleoyl)cholenic acid, a dimethoxytrityl, or a phenoxazine.

In certain embodiments, at least one lipophilic moiety includes a saturated or unsaturated C₄-C₃₀ hydrocarbon chain, and an optional functional group that is hydroxyl, amine, carboxylic acid, sulfonate, phosphate, thiol, azide, or alkyne. Optionally, at least one lipophilic moiety includes a saturated or unsaturated C₆-C₁₈ hydrocarbon chain. Optionally, at least one lipophilic moiety includes a saturated or unsaturated C₁₆ or C₂₂ hydrocarbon chain.

In some embodiments, each lipophilic moiety includes a saturated or unsaturated C₄-C₃₀ hydrocarbon chain, and an optional functional group that is hydroxyl, amine, carboxylic acid, sulfonate, phosphate, thiol, azide, or alkyne. Optionally, each lipophilic moiety includes a saturated or unsaturated C₆-C₁₈ hydrocarbon chain. Optionally, each lipophilic moiety includes a saturated or unsaturated C₁₆ or C₂₂ hydrocarbon chain.

In some embodiments, at least one lipophilic moiety is conjugated to the multi-targeted molecule through a monovalent or branched bivalent or trivalent linker.

In one embodiment, at least one lipophilic moiety is conjugated to one or more nucleotides within the multi-targeted molecule as shown in formula (I)

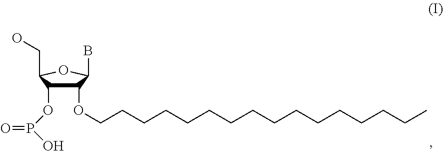

wherein B is a nucleotide base or a nucleotide base analog and the n-hexadecyl chain (“C16 ligand”) is the lipophilic moiety, optionally wherein B is adenine, guanine, cytosine, thymine or uracil. The modification shown in formula (I) is referred to herein as “2′-C16”.

In some embodiments, one or more non-terminal nucleotide positions of the sense strands of the first dsRNA and second dsRNA molecules have the 2′-C16 structure of formula (I), wherein B is a nucleotide base or a nucleotide base analog, optionally wherein B is adenine, guanine, cytosine, thymine or uracil, wherein the n-hexadecyl chain is the lipophilic moiety. Optionally, one or more non-terminal nucleotide positions of the sense strand of the first dsRNA molecule and one or more non-terminal nucleotide positions of the sense strand of the second dsRNA molecule, if present, have the following structure:

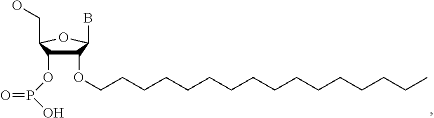

wherein B is a nucleotide base or a nucleotide base analog, optionally wherein B is adenine, guanine, cytosine, thymine or uracil, wherein the n-hexadecyl chain is the lipophilic moiety.

In some embodiments, the multi-targeted molecule includes a first effector molecule that is a RNAi agent and a second effector molecule that is a RNAi agent. In certain embodiments, the first effector molecule is a first dsRNA and the second effector molecule is a second dsRNA. Optionally, the first effector molecule is a first double-stranded siRNA molecule and the second effector molecule is a second double-stranded siRNA molecule.

In some embodiments, the sense strand of the first dsRNA is covalently linked to the sense strand of the second dsRNA. Alternatively, the sense strand of the first dsRNA is covalently linked to the antisense strand of the second dsRNA. In a further alternative, the antisense strand of the first dsRNA is covalently linked to the sense strand of the second dsRNA.

In certain embodiments, each dsRNA includes a lipophilic ligand, e.g., a C16 ligand (also referred to herein as a “2′-C16”, as indicated above), conjugated to a residue that is six nucleotides from the 5′-end of sense strand of the dsRNA (i.e., when numbering nucleotide residues from the 5′-end of the sense strand, wherein the 5′-terminal nucleotide is nucleotide number one, the lipophilic ligand is attached to nucleotide number six). In alternative embodiments, the lipophilic ligand is conjugated to the 3′ end of the sense strand, optionally through a monovalent or branched bivalent or trivalent linker. It is specifically contemplated that a lipophilic ligand can be included in or conjugated to any of the nucleotide positions of the multi-targeted molecules provided in the instant application.

In certain embodiments, at least one lipophilic moiety/ligand is an aliphatic, alicyclic, or polyalicyclic compound. Optionally, the lipophilic moiety is lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In some embodiments, the lipophilic moiety contains a saturated or unsaturated C₄-C₃₀ hydrocarbon chain, and an optional functional group that is hydroxyl, amine, carboxylic acid, sulfonate, phosphate, thiol, azide, or alkyne.

In certain embodiments, at least one lipophilic moiety/ligand contains a saturated or unsaturated C₆-C₁₈ hydrocarbon chain. Optionally, the lipophilic moiety/ligand contains a saturated or unsaturated C₁₆ hydrocarbon chain. In a related embodiment, at least one lipophilic moiety/ligand is conjugated via a carrier that replaces one or more nucleotide(s) of the multi-targeted molecule. In certain embodiments, the carrier is a cyclic group that is pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl; or is an acyclic moiety based on a serinol backbone or a diethanolamine backbone.

In some embodiments, a lipophilic moiety is independently conjugated to position 6 of the sense strand of each dsRNA molecule, counting from the 5′-end of the sense strand of each dsRNA molecule, optionally wherein the lipophilic moiety comprises a saturated or unsaturated C₁₆ or C₂₂ hydrocarbon chain, optionally wherein the lipophilic moiety is a saturated or unsaturated C₁₆ or C₂₂ hydrocarbon chain.

In some embodiments, the lipophilic moiety is conjugated via a bio-cleavable linker. Optionally, the bio-cleavable linker is or comprises DNA, RNA, disulfide, amide, functionalized monosaccharides or oligosaccharides of galactosamine, glucosamine, glucose, galactose, mannose, or combinations thereof.

In some embodiments, the saturated or unsaturated C₁₆ hydrocarbon chain is conjugated to position 6, counting from the 5′-end of the strand.

In certain embodiments, the sense strand of at least one of the at least two dsRNAs is 21 nucleotides in length.

In some embodiments, each of the first dsRNA and the second dsRNA has a sense strand of 19-30 nucleotides in length. Optionally, each of the first dsRNA and the second dsRNA has a sense strand of 21-25 nucleotides in length. Optionally, each of the first dsRNA and the second dsRNA has a sense strand of 21 nucleotides in length.

In some embodiments, each of the first dsRNA and the second dsRNA has an antisense strand of 19-30 nucleotides in length. Optionally, each of the first dsRNA and the second dsRNA has an antisense strand of 21-25 nucleotides in length. Optionally, each of the first dsRNA and the second dsRNA has an antisense strand of 23 nucleotides in length. Optionally, the 3′-end of the antisense strand forms a 3′-overhang of two nucleotides in length with respect to the 5′-end of the sense strand.

In some embodiments, one or more lipophilic moieties are conjugated to one or more of the following internal (non-terminal) positions of one or multiple dsRNAs: non-terminal positions excluding positions 9-12 on the sense strand and all non-terminal positions on the antisense strand. Optionally, one or more lipophilic moieties are conjugated to one or more of the following internal (non-terminal) positions of one or multiple dsRNAs: positions 4-8 and 13-18 on the sense strand, and positions 6-10 and 15-18 on the antisense strand, counting from the 5′end of each strand. Optionally, one or more lipophilic moieties are conjugated to one or more of the following non-terminal positions: positions 5, 6, 7, 15, and 17 on the sense strand, and positions 15 and 17 on the antisense strand, counting from the 5′-end of each strand. In certain embodiments, the sense strand is 21 nucleotides in length and the antisense strand is 23 nucleotides in length.

Optionally, the lipophilic moiety is conjugated to position 21, position 20, position 15, position 7, position 6, or position 2 of the sense strand or position 16 of the antisense strand.

In certain embodiments, the lipophilic moiety is conjugated to position 21, position 20, position 15, or position 6 of the sense strand.

In some embodiments, the lipophilic moiety is conjugated to position 21, position 20, or position 15 of the sense strand.

In some embodiments, the lipophilic moiety is conjugated to position 20 or position 15 of the sense strand.

In some embodiments, the lipophilic moiety is conjugated to position 6 of each sense strand.

In some embodiments, the lipophilic moiety is conjugated to position 16 of the antisense strand.

In some embodiments, the lipophilic moiety/ligand is conjugated to a dsRNA of a multi-targeted molecule via a linker containing an ether, thioether, urea, carbonate, amine, amide, maleimide-thioether, disulfide, phosphodiester, sulfonamide linkage, a product of a click reaction, or carbamate.

In one embodiment, the lipophilic moiety/ligand is conjugated to a nucleobase, sugar moiety, or internucleosidic linkage.

In one embodiment, the multi-targeted molecule includes at least one modified nucleotide that is a 2′-O-methyl modified nucleotide, a 2′-fluoro modified nucleotide, a nucleotide that includes a glycol nucleic acid (GNA) or a nucleotide that includes a vinyl phosphonate. Optionally, the multi-targeted molecule, or each dsRNA of the multi-targeted molecule, includes at least one of each of the following modifications: 2′-O-methyl modified nucleotide, a 2′-fluoro modified nucleotide, a nucleotide comprising a glycol nucleic acid (GNA) and a nucleotide comprising vinyl phosphonate.

In a related embodiment, each dsRNA of the multi-targeted molecule includes at least one modified nucleotide that is a 2′-O-methyl modified nucleotide, a 2′-fluoro modified nucleotide, a nucleotide that includes a glycol nucleic acid (GNA) or a nucleotide that includes a vinyl phosphonate. Optionally, the multi-targeted molecule includes at least one of each of the following modifications: a 2′-O-methyl modified nucleotide, a 2′-fluoro modified nucleotide, a 2′-C16 moiety and a phosphorothioate internucleoside linkage.

In some embodiments, each dsRNA includes at least one phosphorothioate or methylphosphonate internucleotide linkage. Optionally, each dsRNA includes between two and eight phosphorothioate or methylphosphonate internucleotide linkages. Optionally, the phosphorothioate or methylphosphonate internucleotide linkages are positioned in each dsRNA at the ultimate and penultimate internucleoside linkages at one or more of the following locations: the 5′-terminus of the sense strand, the 3′-terminus of the sense strand, the 5′-terminus of the antisense strand, the 3′-terminus of the antisense strand, and combinations thereof. Optionally, each dsRNA includes six phosphorothioate or methylphosphonate internucleotide linkages positioned at the ultimate and penultimate internucleoside linkages of the 5′-terminus of the sense strand, the 3′-terminus of the sense strand and the 3′-terminus of the antisense strand.

In certain embodiments, all or substantially all of the nucleotides of each dsRNA includes at least one modification that is a 2′-O-methyl modification, a 2′-fluoro modification or a 2′-C₆-C₁₈ hydrocarbon chain modification.

In another embodiment, the multi-targeted molecule includes a pattern of modified nucleotides as provided herein (e.g., in FIGS. 3A, 4A, 5A, 6A, 7A, 8A and 9A), optionally wherein locations of 2′-C16 (or other lipophilic moiety/ligand), 2′-O-methyl, phosphorothioate and 2′-fluoro modifications are irrespective of the individual nucleotide base sequences of the displayed RNAi agents.

In some embodiments, the sense strand of a first dsRNA has a 5′-end and is connected at its 3′-end to a linker, wherein the linker connects to the 5′-end of a single-stranded nucleic acid agent or second dsRNA molecule. Optionally, the linker connects to the 5′ end of a sense strand of the single-stranded nucleic acid agent or second dsRNA molecule.

In certain embodiments, the sense strands of the dsRNAs are 21 nucleotides in length and are connected by a linker.

In some embodiments, the linker that connects the first dsRNA molecule and the single-stranded nucleic acid agent or second dsRNA molecule is a nucleic acid linker or a carbohydrate or other organic polymer linker. Optionally, the linker is cleavable.

In certain embodiments, the linker connecting the effector molecules includes one or more of the following:
—(CH₂)₁₂— (“C12 linker” or “Q50”),
—(CH₂)₆—S—S—(CH₂)₆— (“C6-S—S—C6 linker” or “Q51”),
Q151,
Q173,
—CH₂CH₂O—(CH₂CH₂)ₙ—CH₂CH₂O—CH₂CH₂O—, wherein n is 0 or 1-20;
—(CH₂)₉—(CH₂)ₙ—CH₂— wherein n is 0 or 1-20;
mono-, di-, tri-, tetra-, penta- or polyprolinol, optionally conjugated with a ligand; or
mono-, di-, tri-, tetra-, penta- or poly (e.g., mono-, di-, tri-, tetra-, penta- or poly[4-hydroxyprolinol]), optionally conjugated with a ligand.

In some embodiments, the linker that connects the effector molecules (e.g., dsRNAs or first dsRNA and single-stranded nucleic acid agent) is an organic polymer linker such as an aliphatic saturated or unsaturated alkyl chain, a (poly)ethylene glycol chain, including diethylene glycol, triethylene glycol, tetra-, penta-, hexa-, hepta-, octa-, nona-, and/or deca-ethylene glycol.

In certain embodiments, the linker is a bio-cleavable linker that is or includes a DNA, RNA, disulfide, amide, or functionalized monosaccharide or oligosaccharide of galactosamine, glucosamine, glucose, galactose, or mannose, or combinations thereof. In some embodiments, the bio-cleavable linker is a combination of an organic polymer linker, such as an aliphatic saturated or unsaturated alkyl chain, a (poly)ethylene glycol chain (including diethylene glycol, triethylene glycol, tetra-, penta-, hexa-, hepta-, octa-, nona-, and/or deca-ethylene glycol, and/or includes a DNA, RNA, disulfide, amide, or functionalized monosaccharide or oligosaccharide of galactosamine, glucosamine, glucose, galactose, or mannose, or combinations thereof.

In certain embodiments, the organic polymer linker includes one or more of the following: an aliphatic saturated or unsaturated alkyl chain, and a (poly)ethylene glycol chain, including diethylene glycol, triethylene glycol, tetra, penta, hexa, hepta, octa, nona, deca ethylene glycol, and glycerol and/or aminoalkyl ethers thereof.

In certain embodiments, the bis-linker connecting the first strand (circular or substantially circular sense strand, or circular or substantially circular antisense strand) nucleotide sequences comprises a moiety selected from the group consisting of

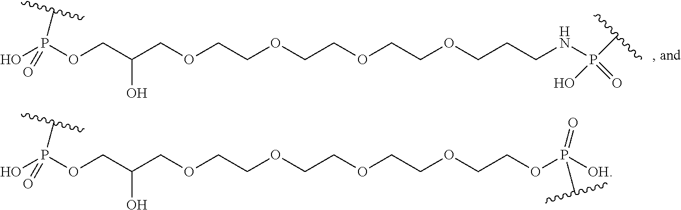

In some embodiments, the organic polymer linker includes a DNA, RNA, disulfide, amide, functionalized monosaccharide or oligosaccharide of galactosamine, glucosamine, glucose, galactose, or mannose, or a combination thereof.

In certain embodiments, the linker that connects the first dsRNA molecule and the single-stranded nucleic acid agent or second dsRNA molecule is selected from the following: —(CH₂)₁₂— (C12 linker or Q50), —(CH₂)₆—S—S—(CH₂)₆— (C6-S—S—C6 linker or Q51), Q151, Q173, —CH₂CH₂O—(CH₂CH₂)ₙ—CH₂CH₂O—CH₂CH₂O—, wherein n is 0 or 1-20; —(CH₂)₉—(CH₂)ₙ—CH₂— wherein n is 0 or 1-20; mono-, di-, tri-, tetra-, penta- or polyprolinol, optionally conjugated with a ligand; mono-, di-, tri-, tetra-, penta- or polyhydroxyprolinol, optionally conjugated with a ligand.

In some embodiments, a bio-cleavable linker is selected from the following:

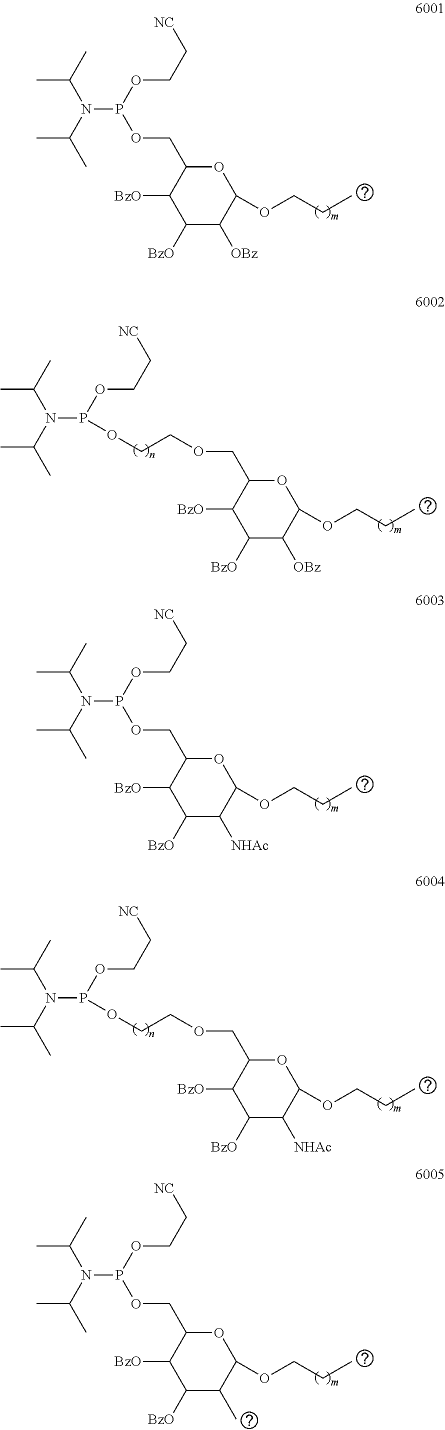

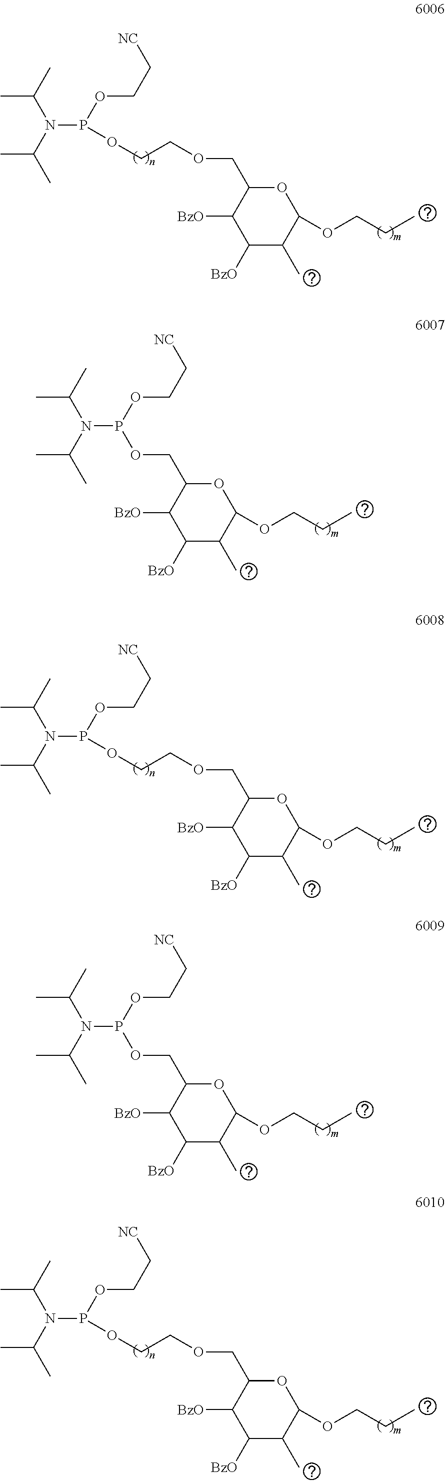

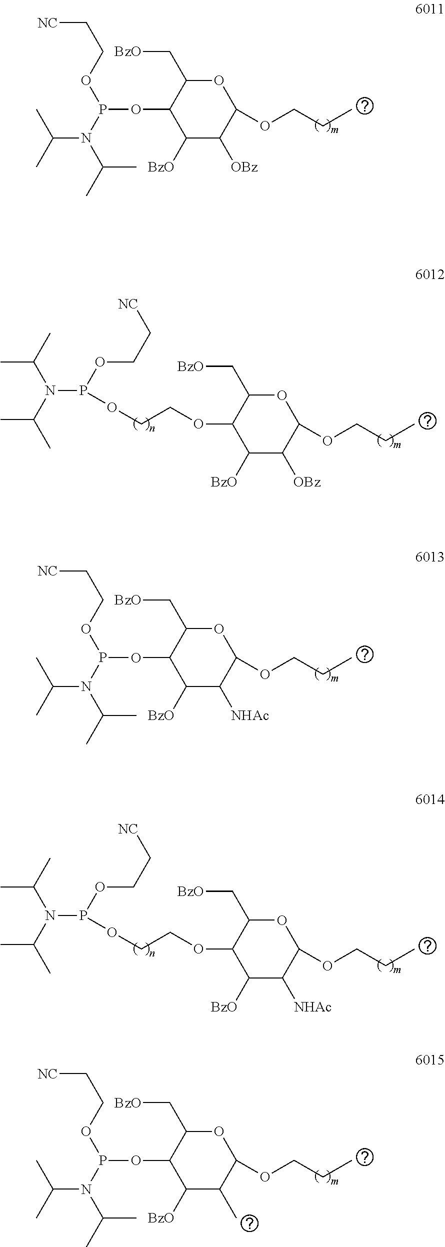

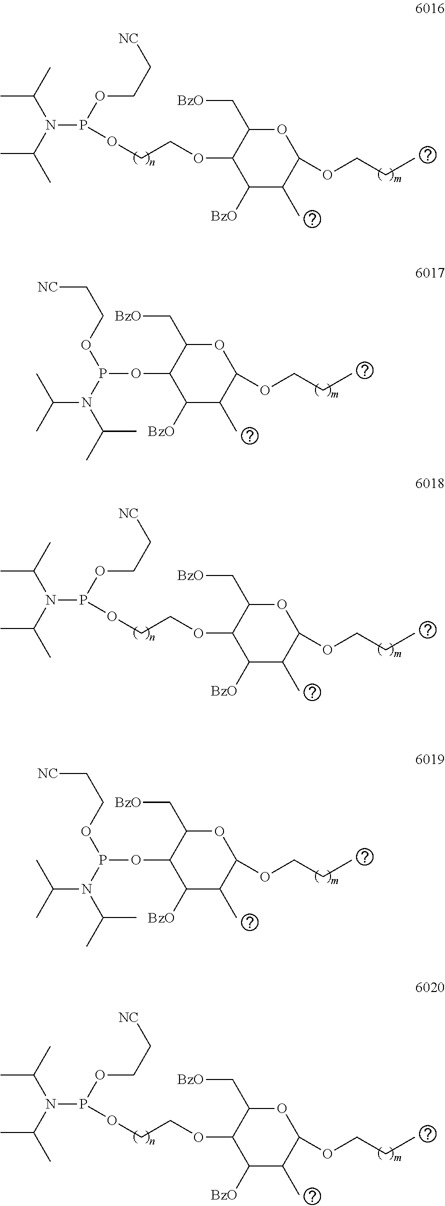

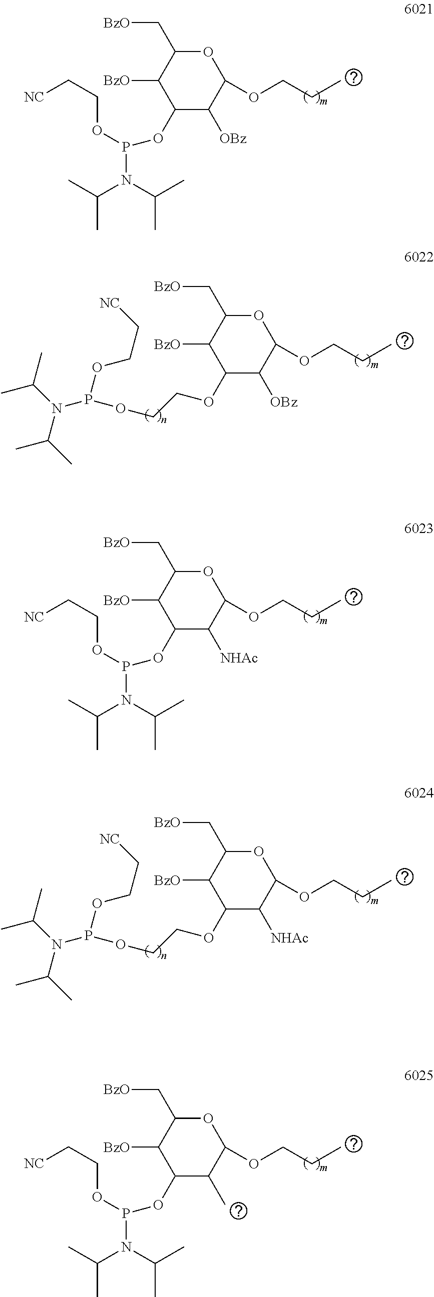

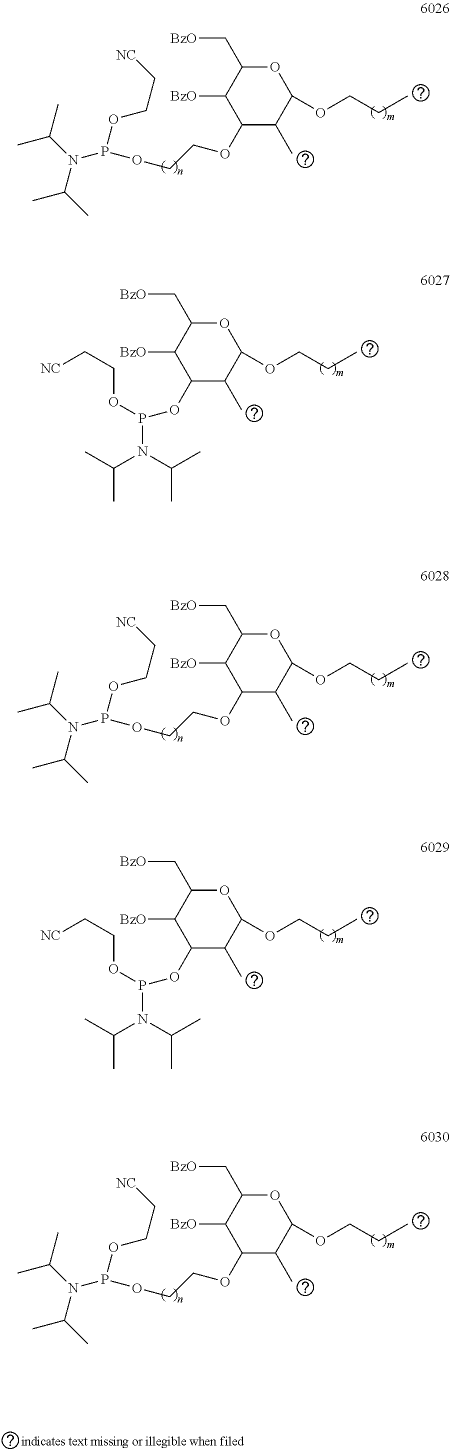

wherein n=1-12 and m=1-12, various carbohydrates (galactose, galactosamine, glucose, glucosamine, mannose, mannosamine derivatives or pentose derivatives).

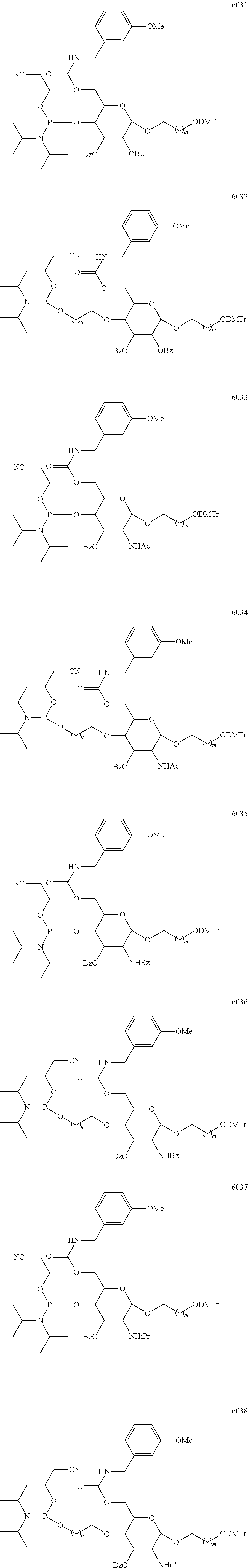

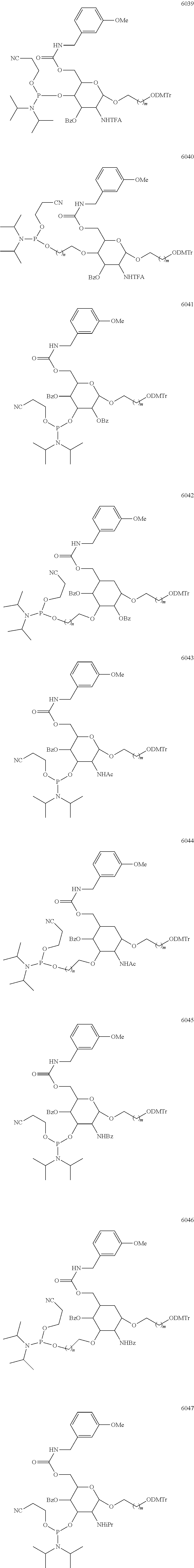

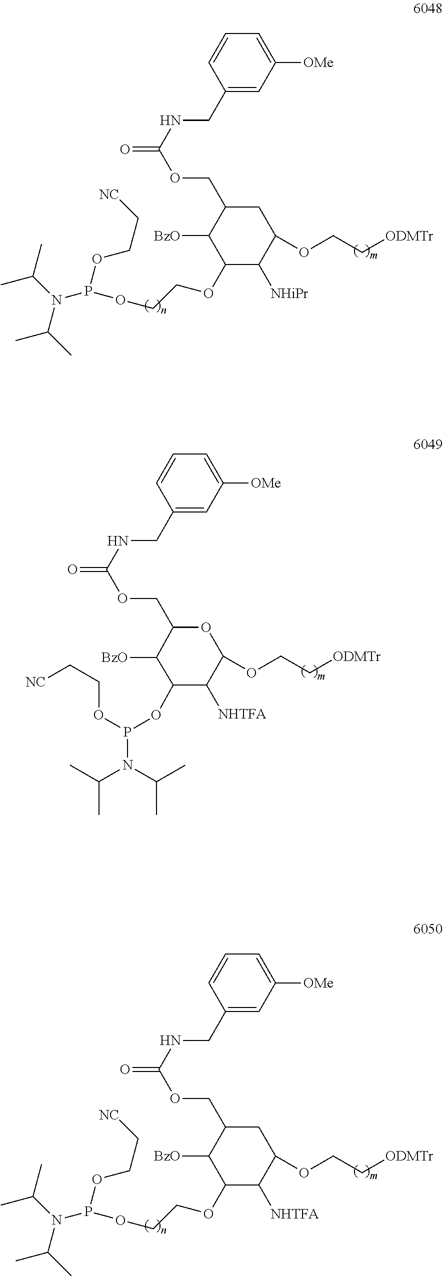

wherein n=1-12 and m=1-12, various modified carbohydrates (galactose, galactosamine, glucose, glucosamine, mannose, mannosamine derivatives or pentose derivatives).

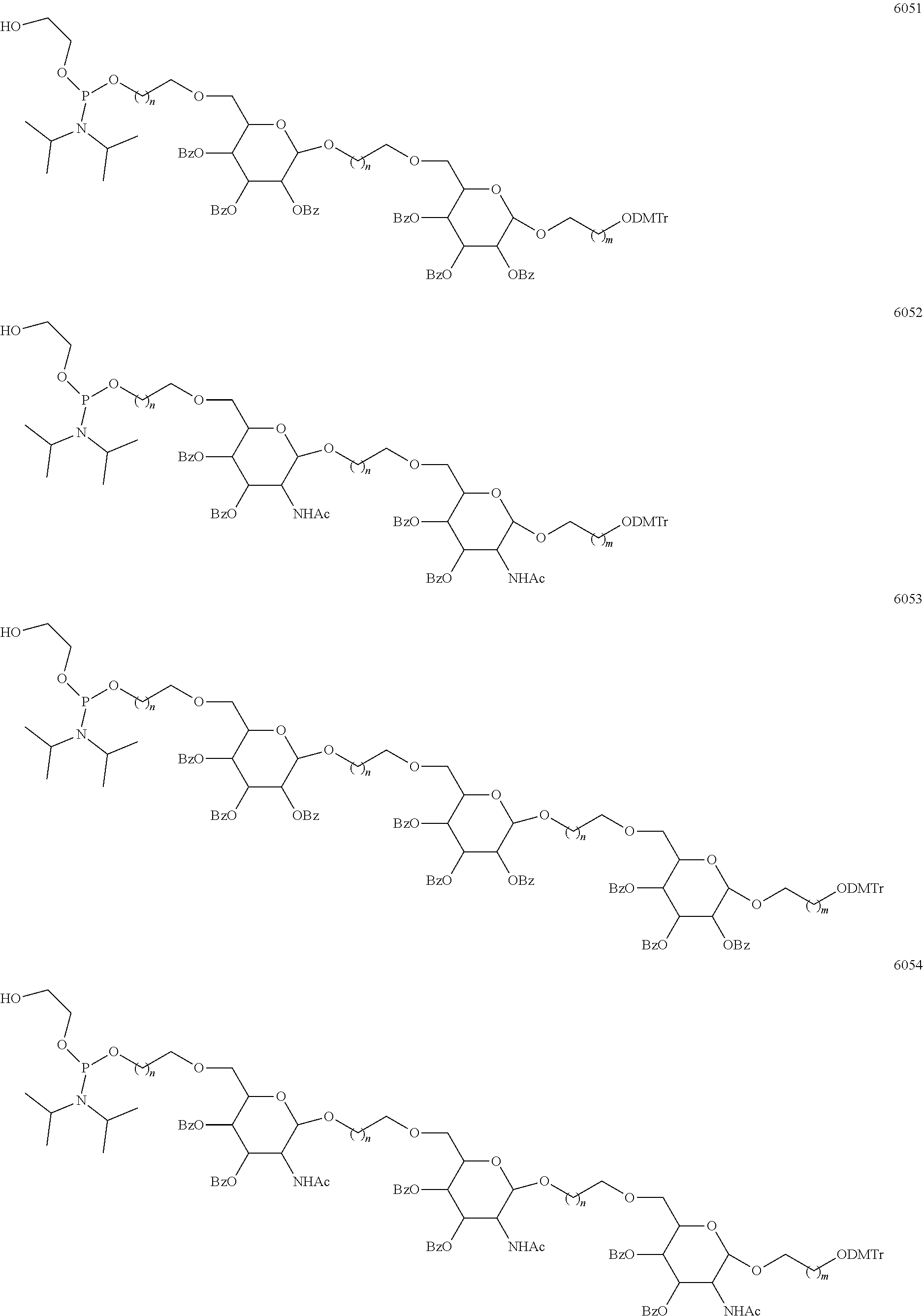

wherein n=1-12 and m=1-12, various modified carbohydrates (di or tri saccharides of galactose, galactosamine, glucose, glucosamine, mannose, mannosamine derivatives).

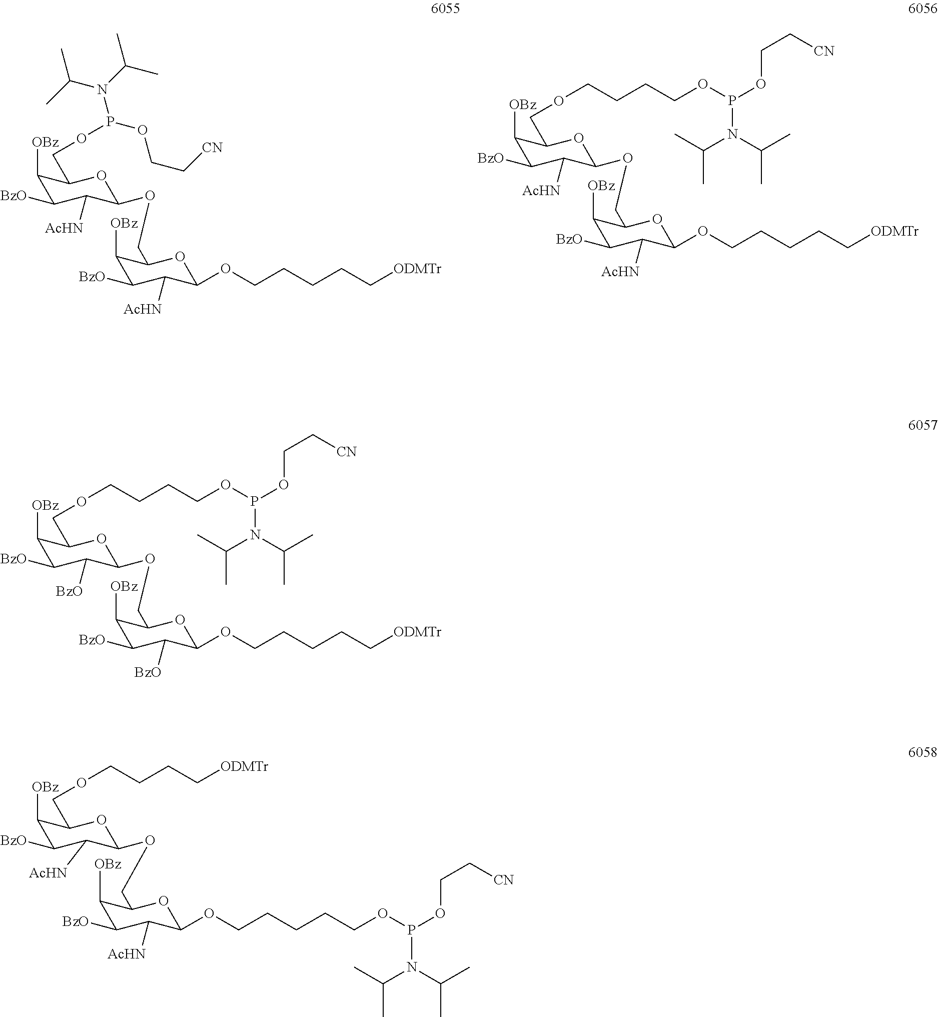

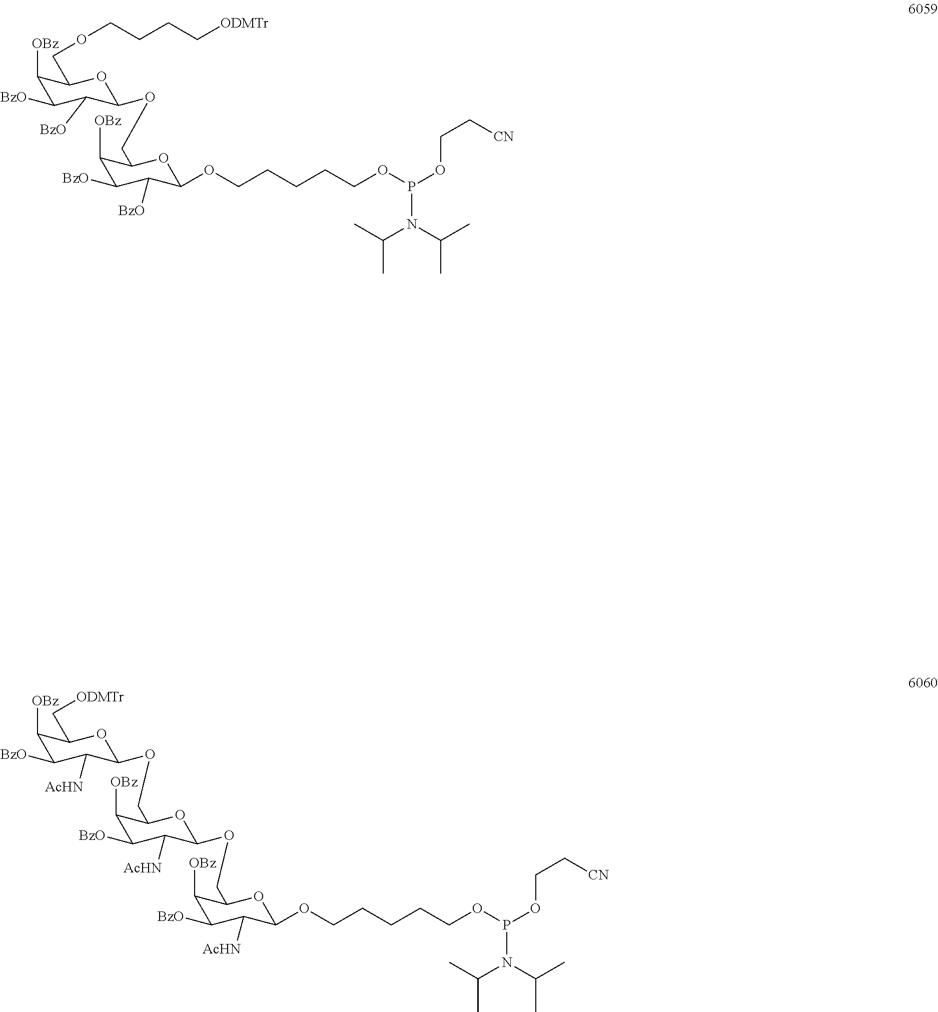

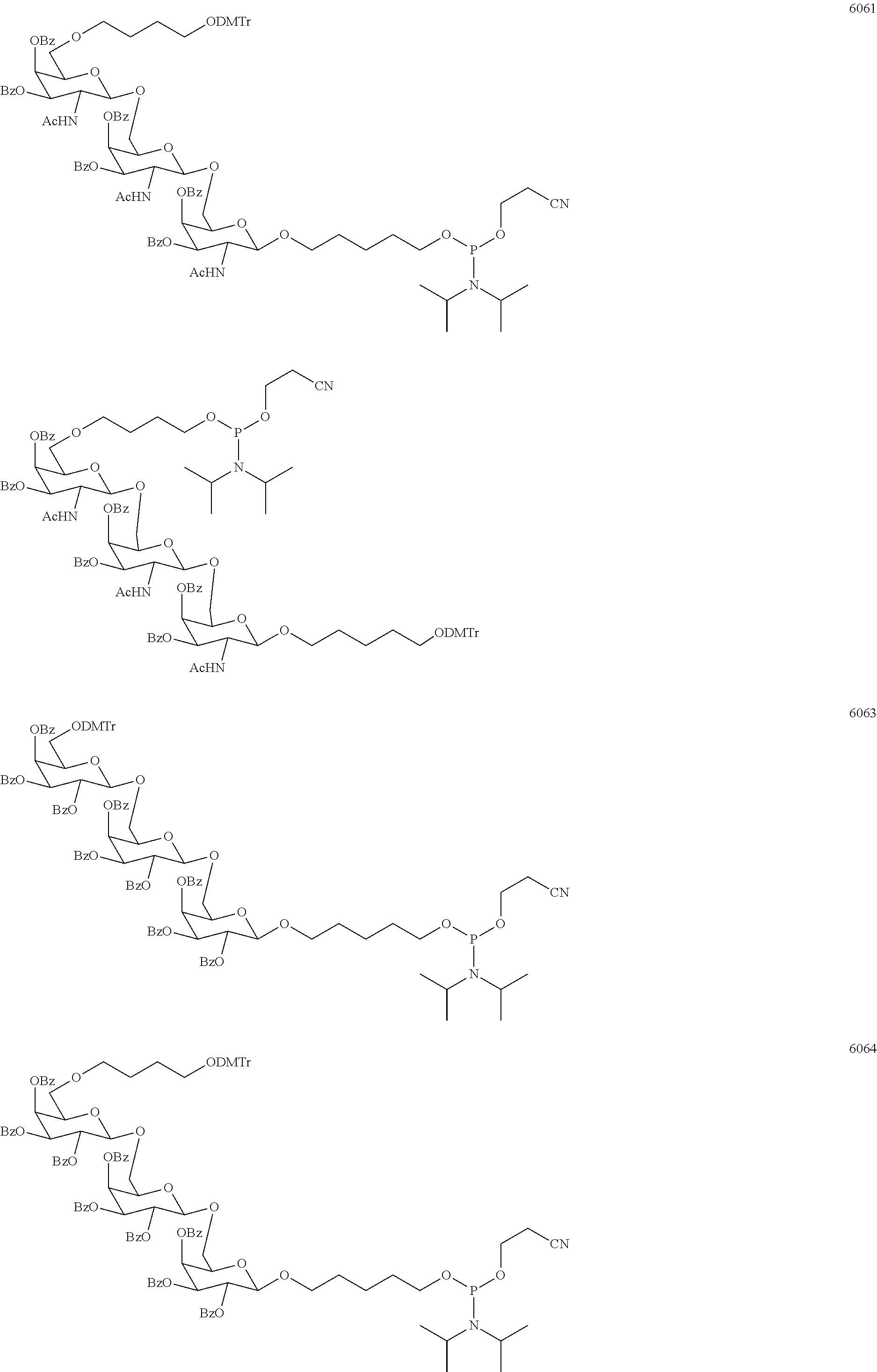

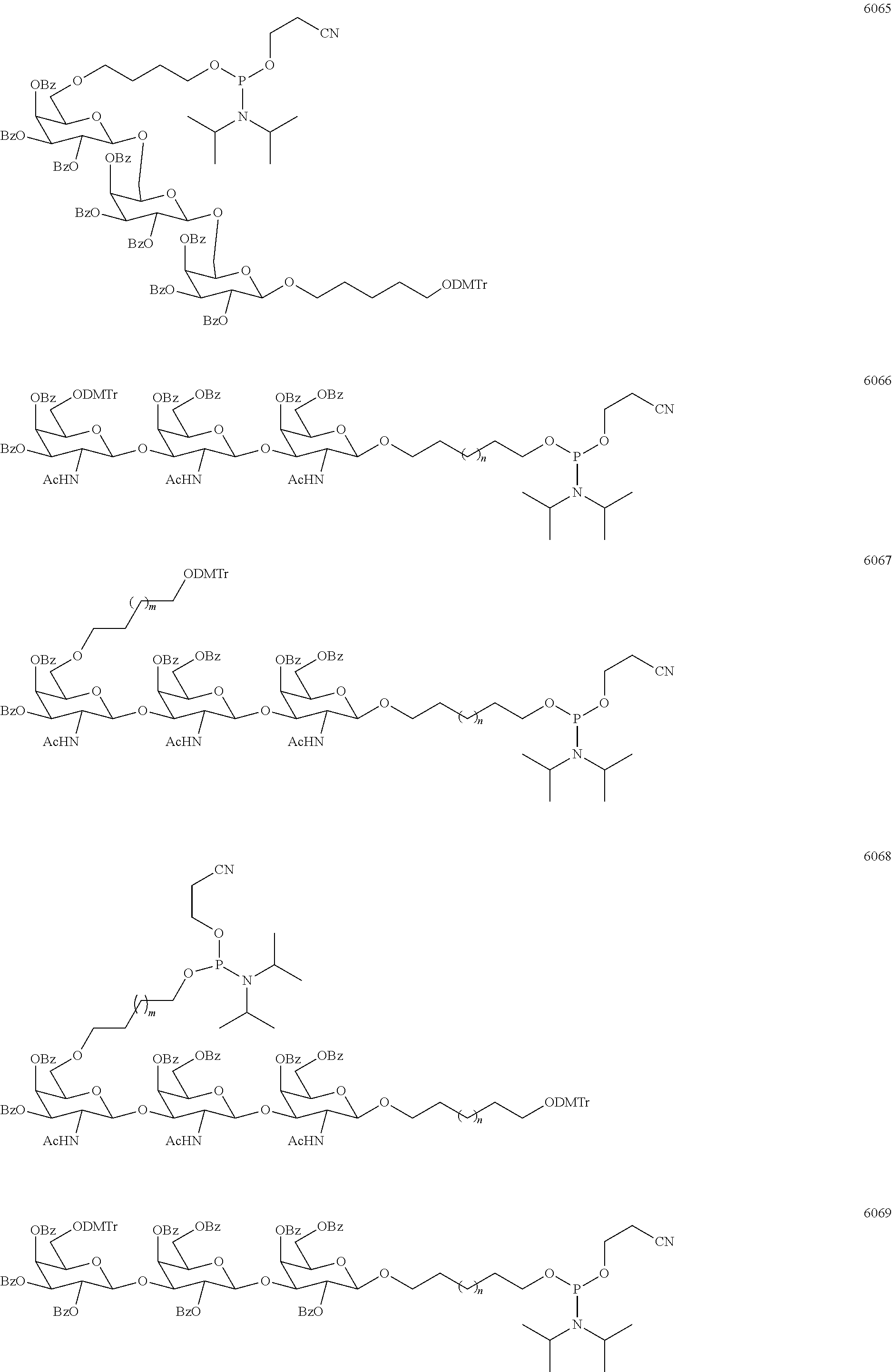

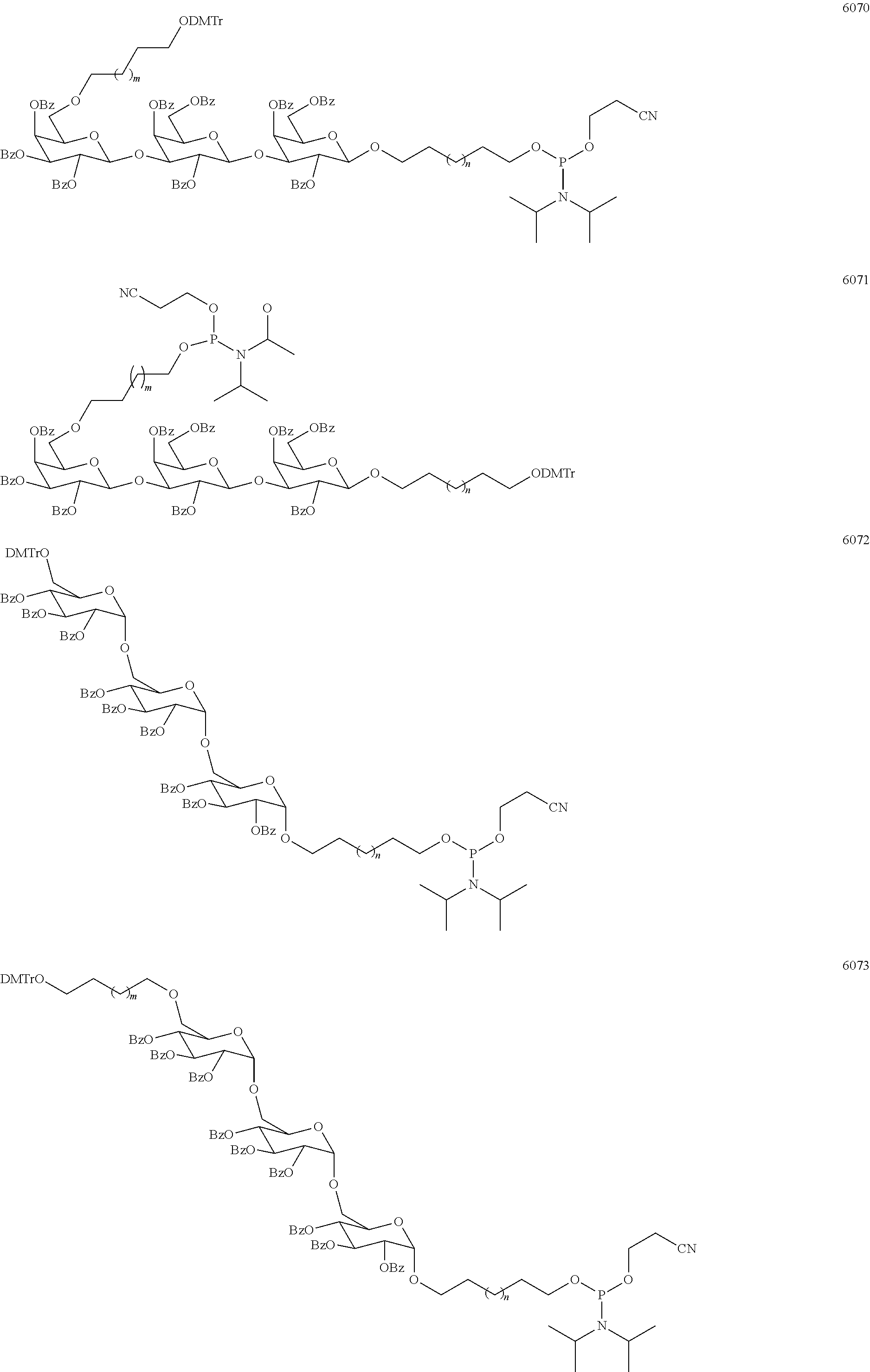

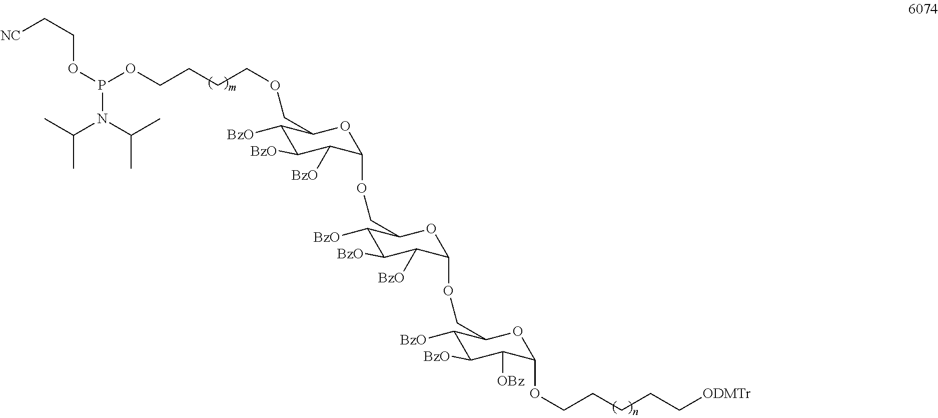

wherein n=1-12 and m=1-12, various modified carbohydrates (di or tri saccharides of galactose, galactosamine, glucose, glucosamine, mannose, mannosamine derivatives).

In certain embodiments, the linker is a polynucleotide. Optionally, the linker is a polynucleotide that includes a deoxyribonucleotide sequence, a ribonucleotide sequence, or both. Optionally, the linker is a polynucleotide having a modified ribonucleotide sequence. Optionally the linker is a polynucleotide having one or more of the following modifications: 2′-O-methyl ribonucleotide or 2′-fluoro-ribonucleotide; 2′-5′-linked nucleotide with a 3′-modification (3′-ribo, 3′-O-methyl, 3′-deoxy, 3′-fluoro). Optionally, the linker is a polynucleotide having one or more of the following modifications: glycol nucleic acid (GNA), locked nucleic acid (LNA), hexanol nucleic acid (HNA), abasic ribose, abasic deoxyribose, abasic hydroxyprolinol. Optionally, all nucleic acid linker nucleotides are the same type of nucleotide. Optionally, the linker entirely includes 2′-O-methyl nucleotides, entirely includes 2′-fluoro nucleotides, or entirely includes deoxyribonucleotides.

In some embodiments, the linker is of n nucleotides in length. Optionally, the length of the longest strand (i.e., the first strand, e.g., the combined/linked sense strands of the respective dsRNAs) of the multi-targeted molecule is equal to the length of the first dsRNA+n+the length of the second dsRNA. Optionally, wherein there are only two dsRNAs each having respective sense strands of 21 nucleotides in length joined by a polynucleotide linker of n nucleotides in length, the total length of the longest strand (i.e., the first strand) of the multi-targeted molecule is 42+n nucleotides. In certain embodiments, the polynucleotide linker is two or more nucleotides in length. Optionally, the polynucleotide linker is three or more nucleotides (e.g., 3, 4, 5, 6, 7, 8, 9, 10 or more nucleotides) in length. In certain embodiments, the linker that connects the dsRNAs is a nucleic acid linker of between one and 15 nucleotides in length. Optionally, the linker is of between two and five nucleotides in length. Optionally, the linker is three or four nucleotides in length. Optionally, the linker is three nucleotides in length. In a related embodiment the total length of the longest strand (i.e., the first strand) of the multi-targeted molecule is 45 nucleotides.

In certain embodiments, the linker that connects the first dsRNA molecule and the single-stranded nucleic acid agent or second dsRNA molecule includes one or more of the following sequences: UUU, 2′-O-methyl-UUU (uuu) and 2′-fluoro-UUU (UfUfUf) and (dT)n, wherein n is 1-20, such as dTdTdT.

In some embodiments, the multi-targeted molecule includes two nucleic acid dsRNAs, wherein the sense strand of each dsRNA is 21 nucleotides in length, the antisense strand of each dsRNA is 23 nucleotides in length, the linker connecting the dsRNAs is a nucleic acid linker of three nucleotides in length that connects the sense strands of each dsRNA, and a lipophilic moiety is conjugated to position 6 of the sense strand of each dsRNA.

In some embodiments, the linker connecting the two siRNAs includes the nucleotide sequence UUU or (dT)n, wherein n is 1-20. In certain embodiments, the linker that connects the dsRNAs includes one or more of the following sequences: dTdTdT, UUU, 2′-O-methyl-UUU (uuu) and 2′-fluoro-UUU (UfUfUf).

In certain embodiments, the linker that connects the first dsRNA molecule and the single-stranded nucleic acid agent or second dsRNA molecule is a polynucleotide having a modified ribonucleotide sequence. Optionally, the linker includes a polynucleotide having one or more of the following modifications: a 2′-O-methyl ribonucleotide modification, a 2′-fluoro-ribonucleotide modification, a 2′-5′-linked nucleotide with different 3′-modification (3′-ribo, 3′-O-methyl, 3′-deoxy, 3′-fluoro), a glycol nucleic acid (GNA) modification, a locked nucleic acid (LNA) modification, a hexanol nucleic acid (HNA) modification, an abasic ribose modification, an abasic deoxyribose modification, and an abasic hydroxyprolinol modification.

In some embodiments, all nucleic acid nucleotides of the linker that connects the first dsRNA molecule and the single-stranded nucleic acid agent or second dsRNA molecule are the same type of nucleotide. Optionally, the linker entirely includes 2′-O-methyl nucleotides, entirely includes 2′-fluoro nucleotides or entirely includes deoxyribonucleotides.

In some embodiments, the linker that connects the first dsRNA molecule and the single-stranded nucleic acid agent or second dsRNA molecule is an endosomal cleavable linker or a protease cleavable linker. Optionally, the linker is a carbohydrate linker and the linker is cleaved at least 1.25 times faster in the cell (or under in vitro conditions selected to mimic intracellular conditions) as compared to blood or serum (or under in vitro conditions selected to mimic extracellular conditions).

In some embodiments, the linker that connects the first dsRNA molecule and the single-stranded nucleic acid agent or second dsRNA molecule is selected from among the following:

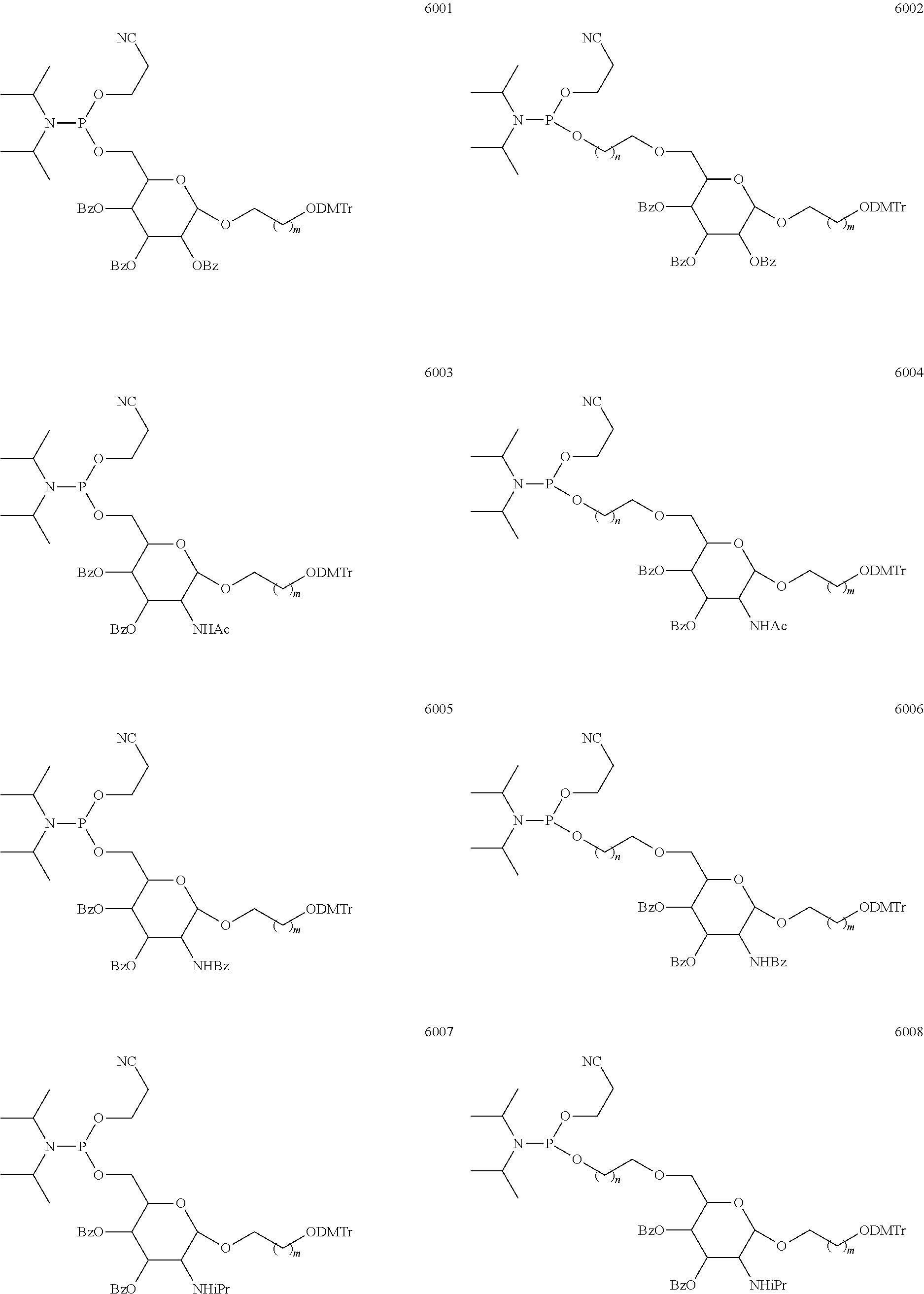

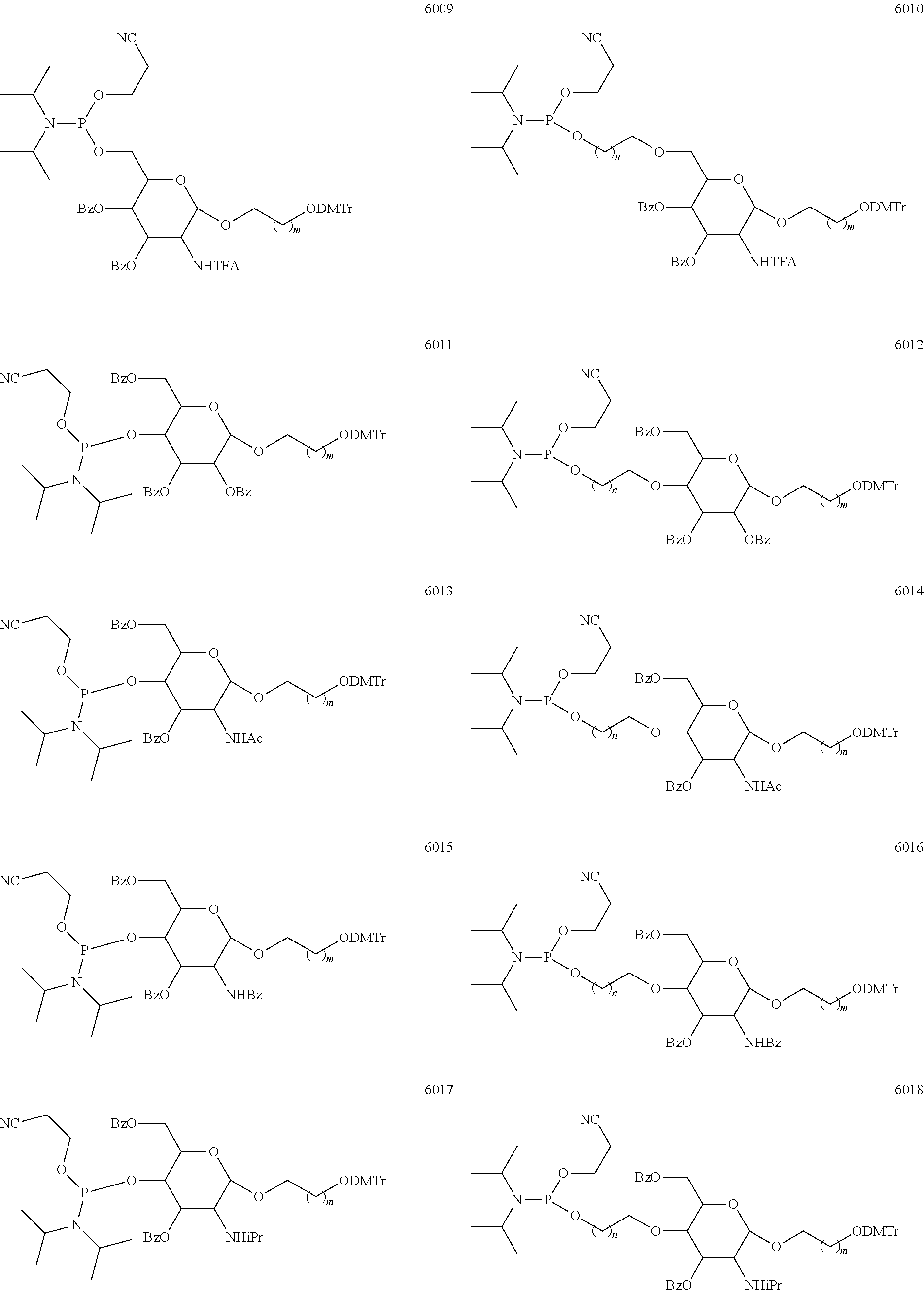

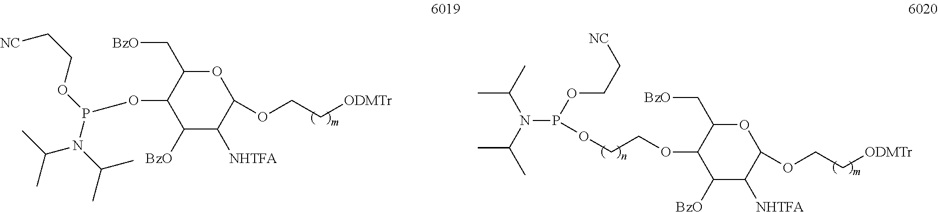

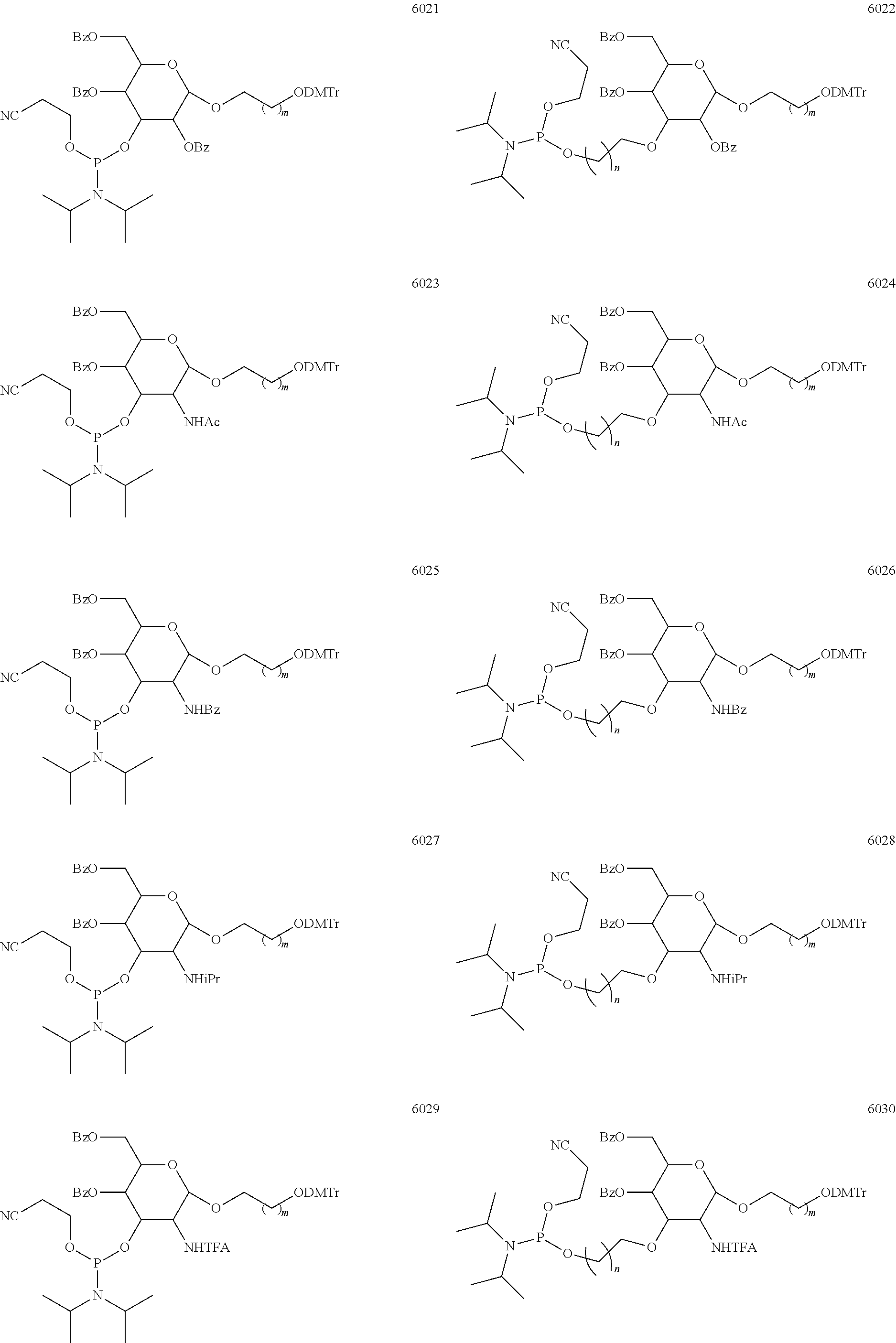

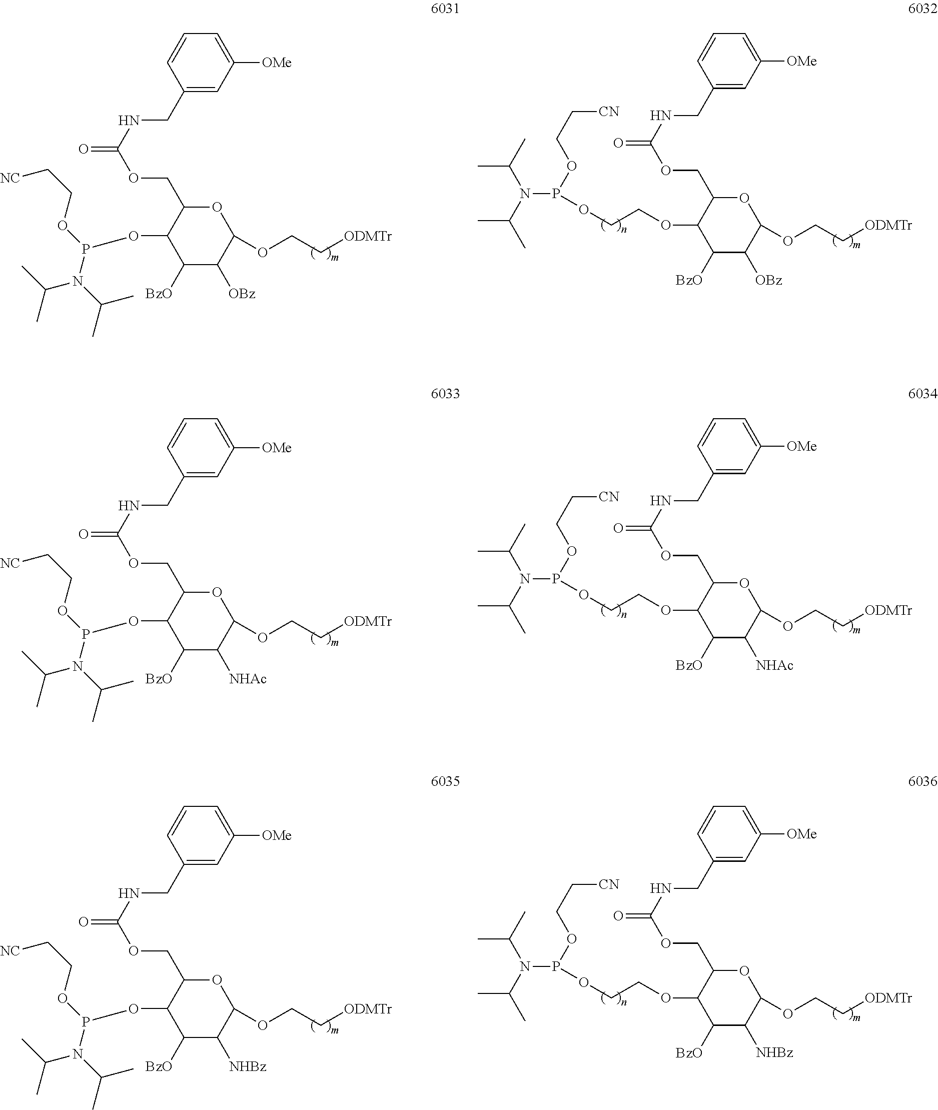

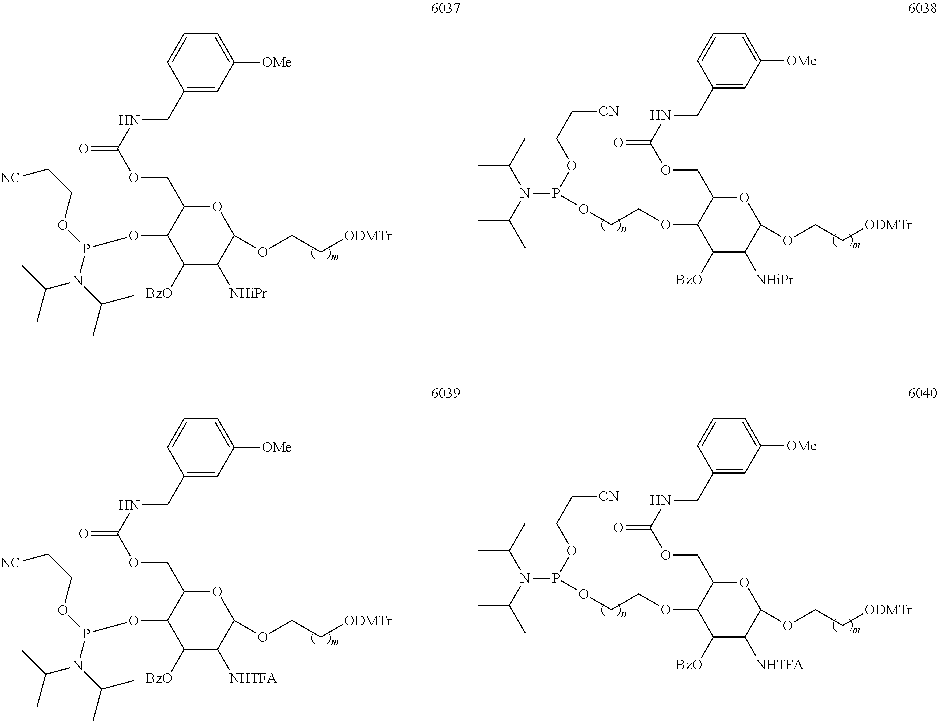

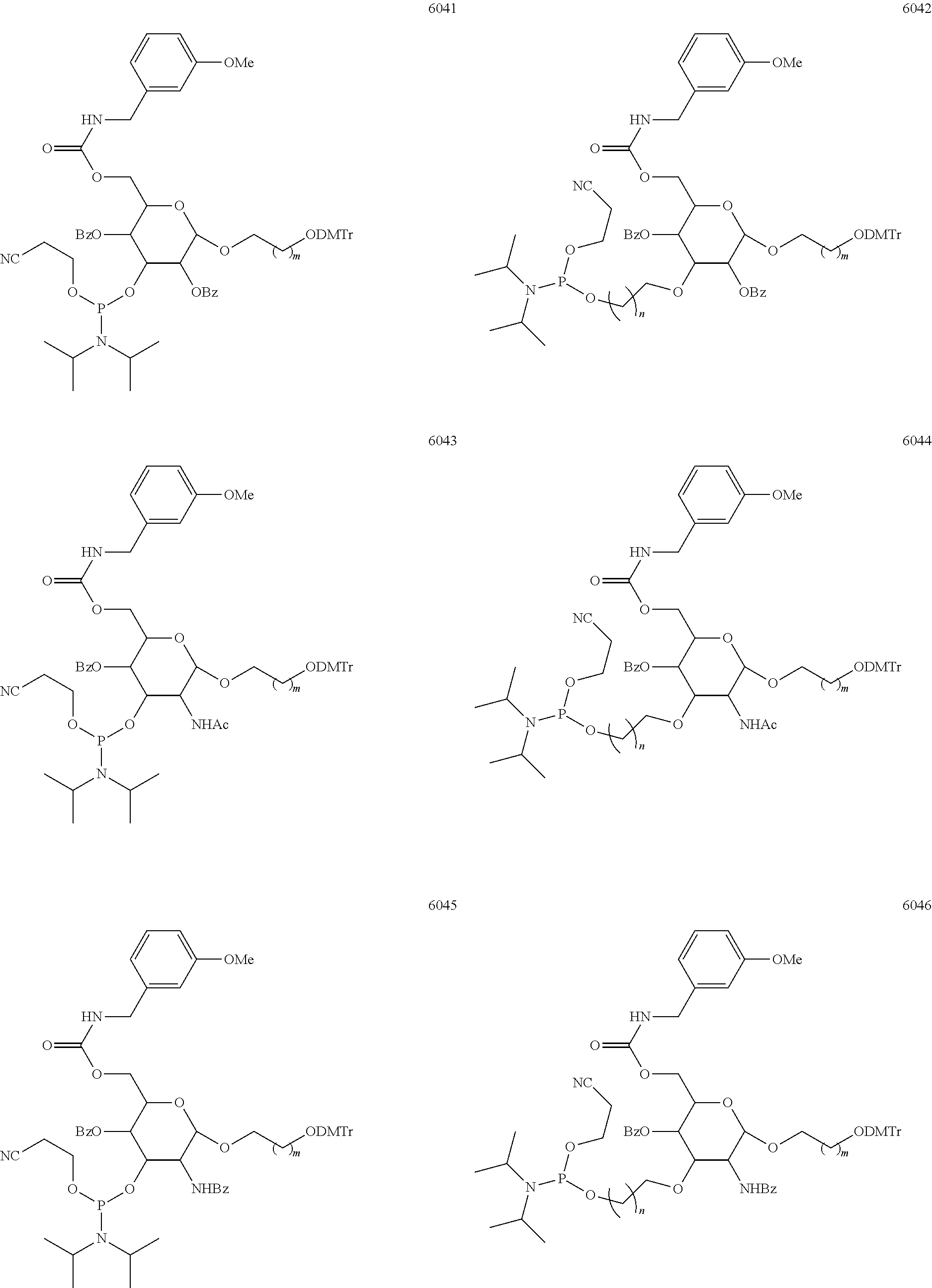

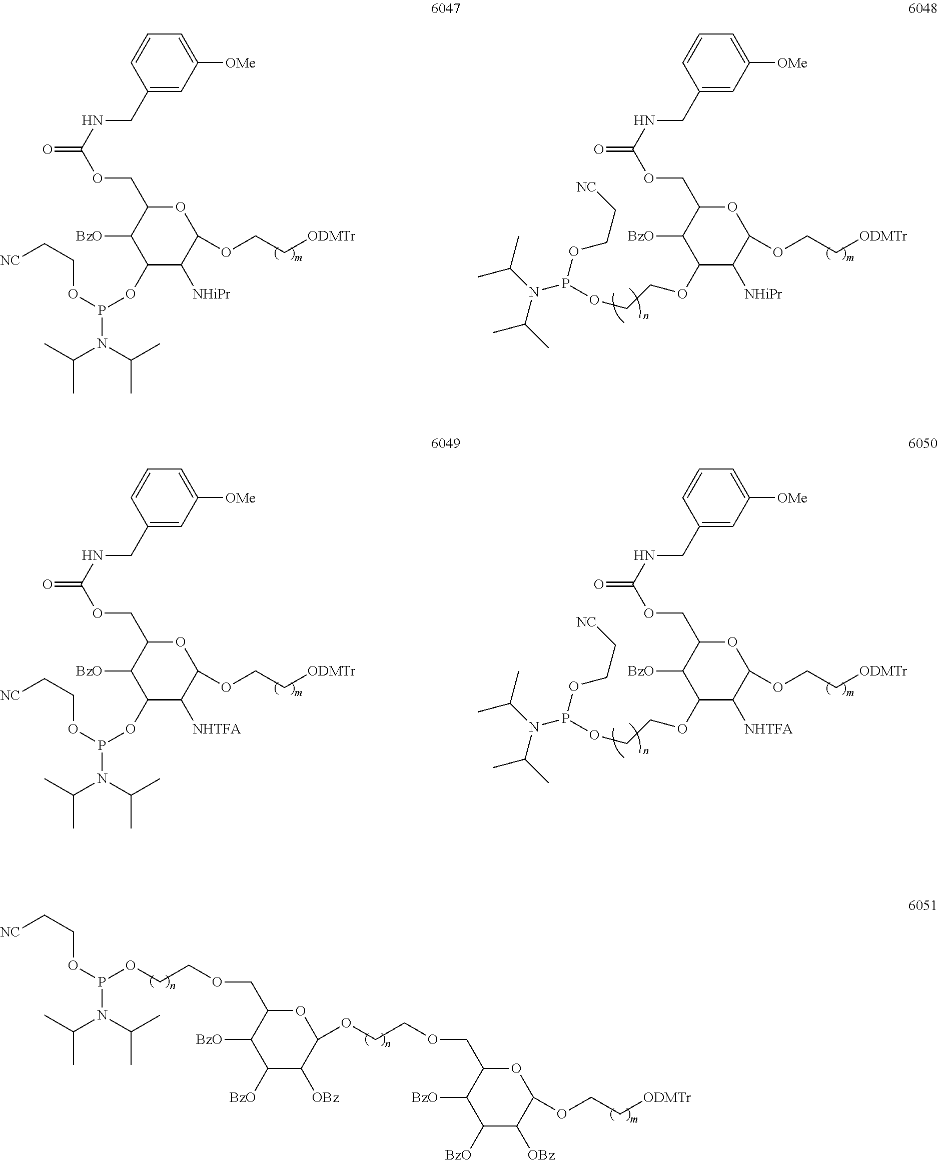

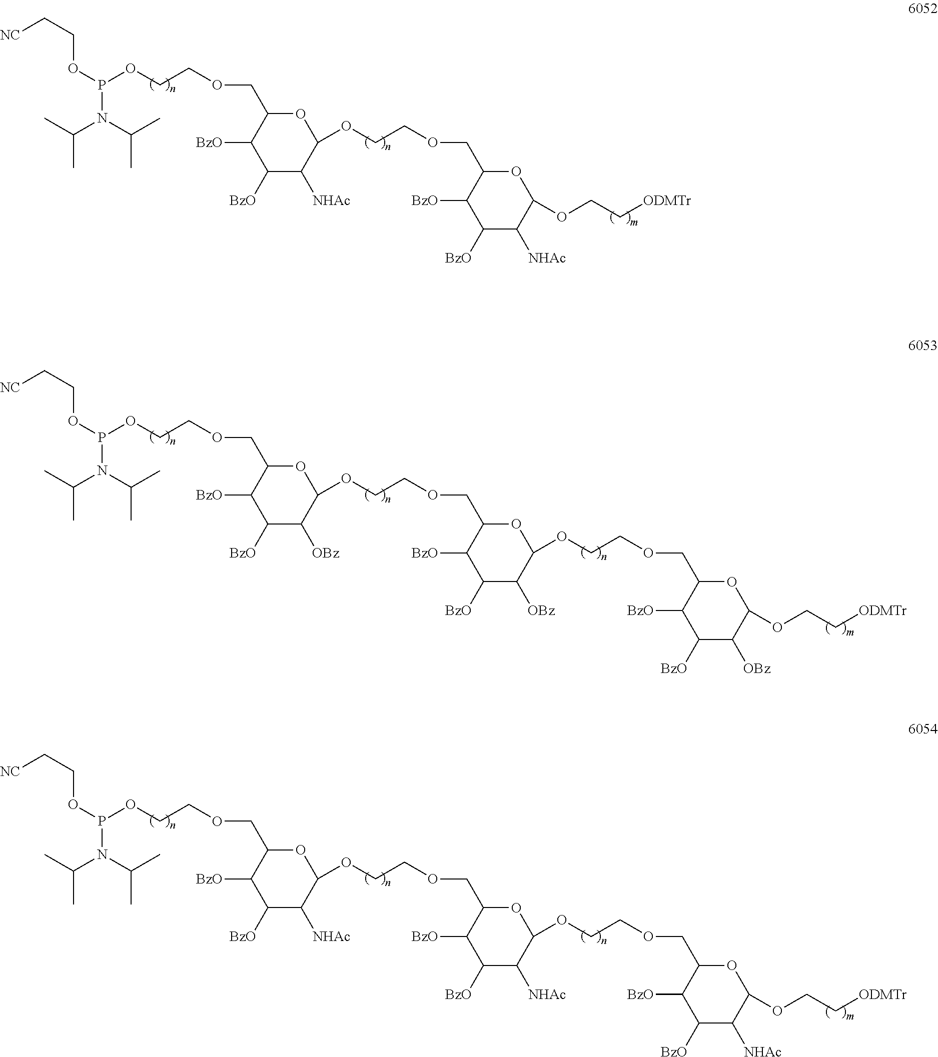

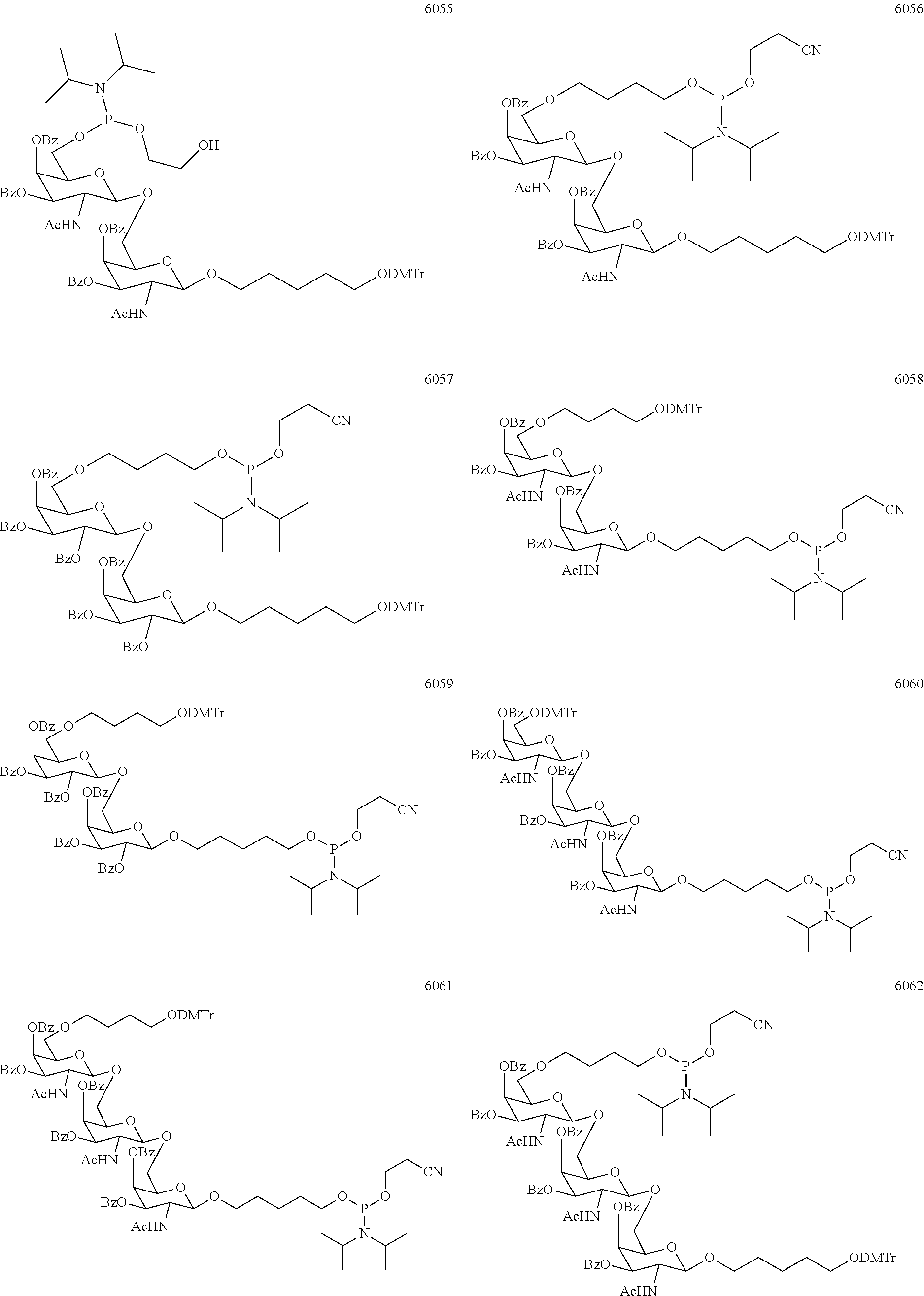

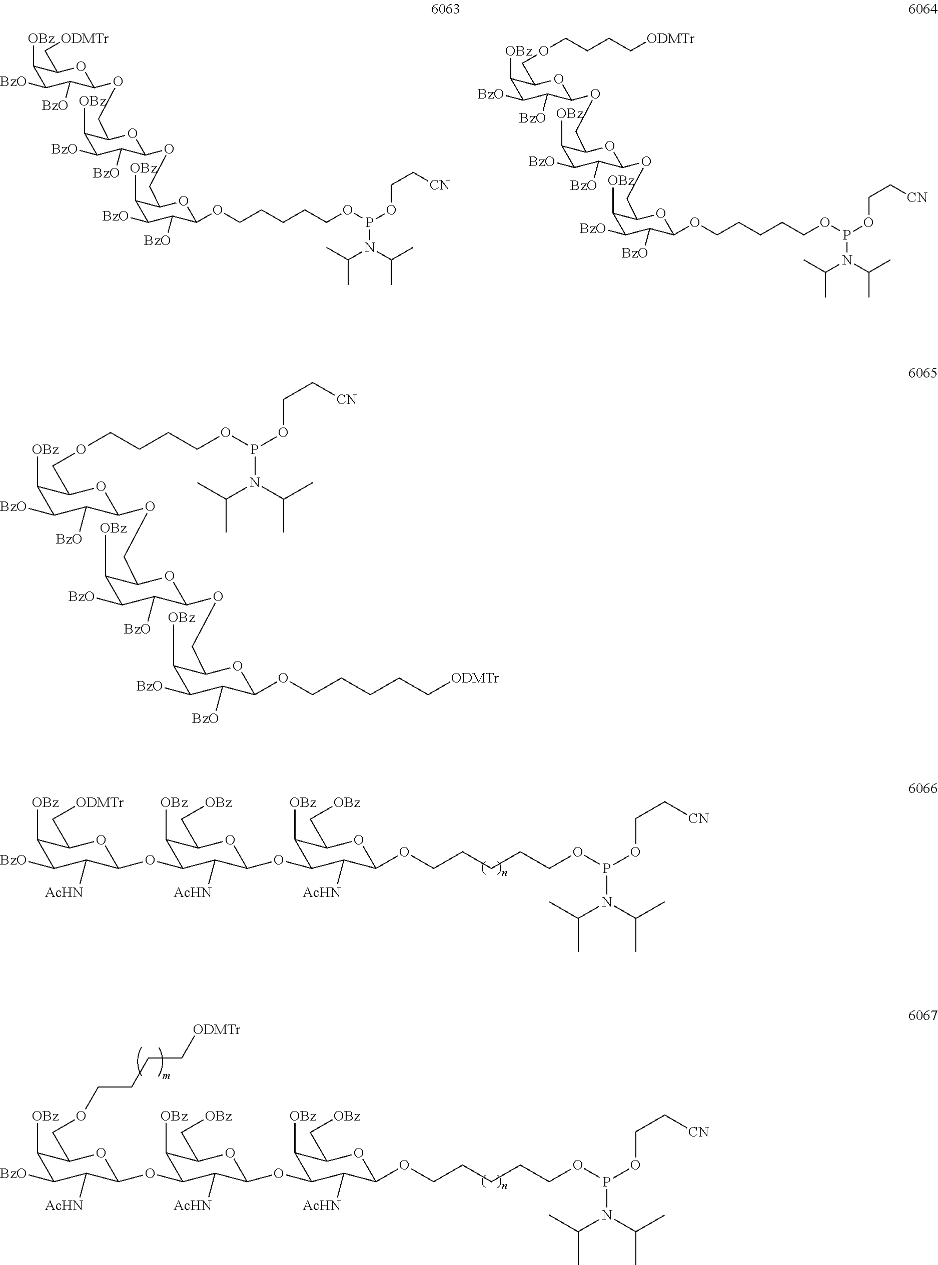

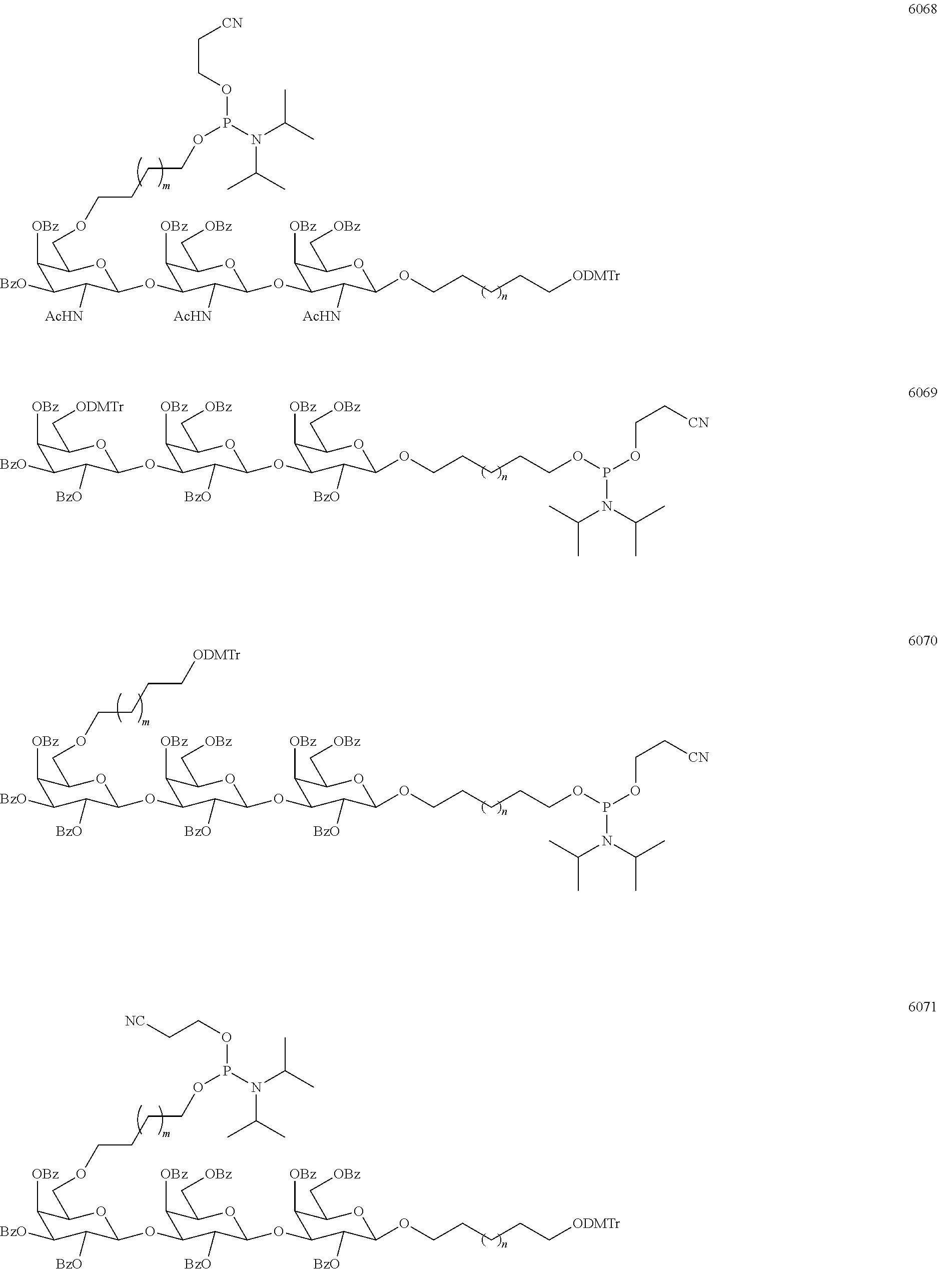

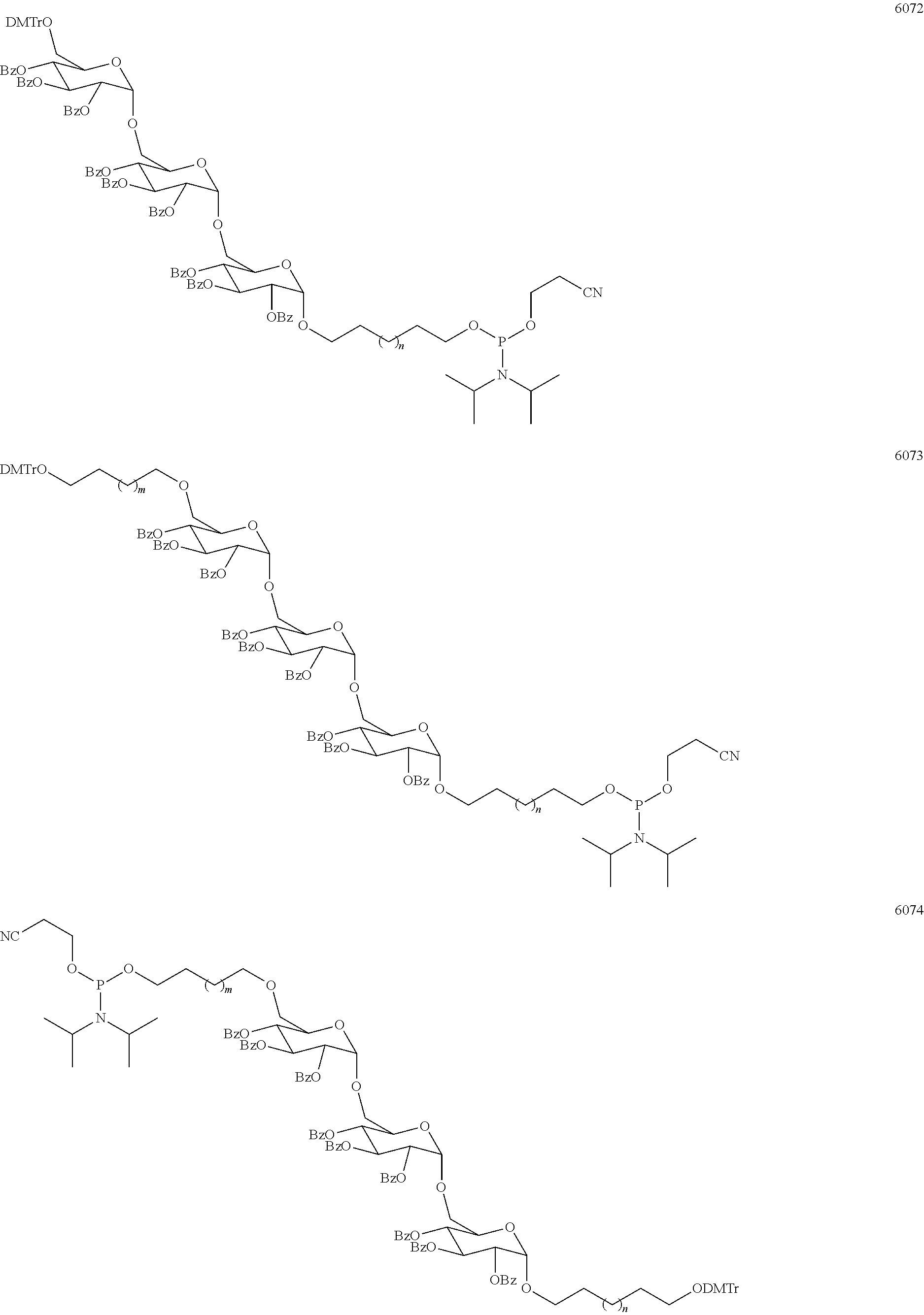

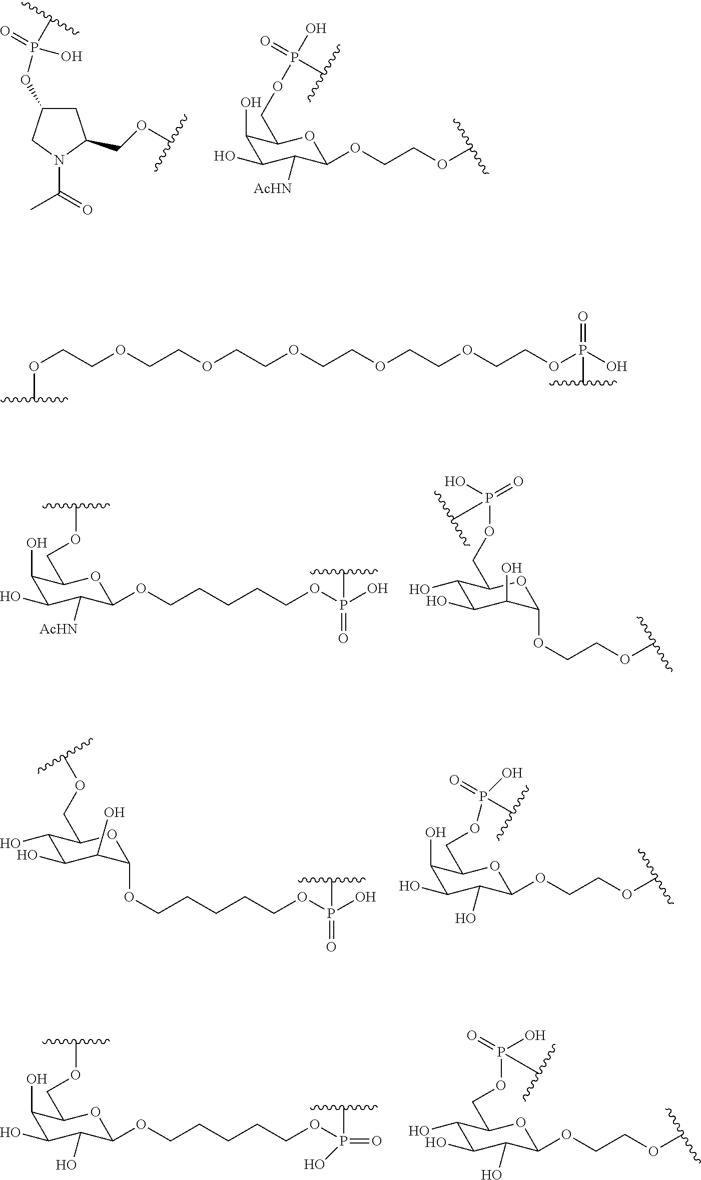

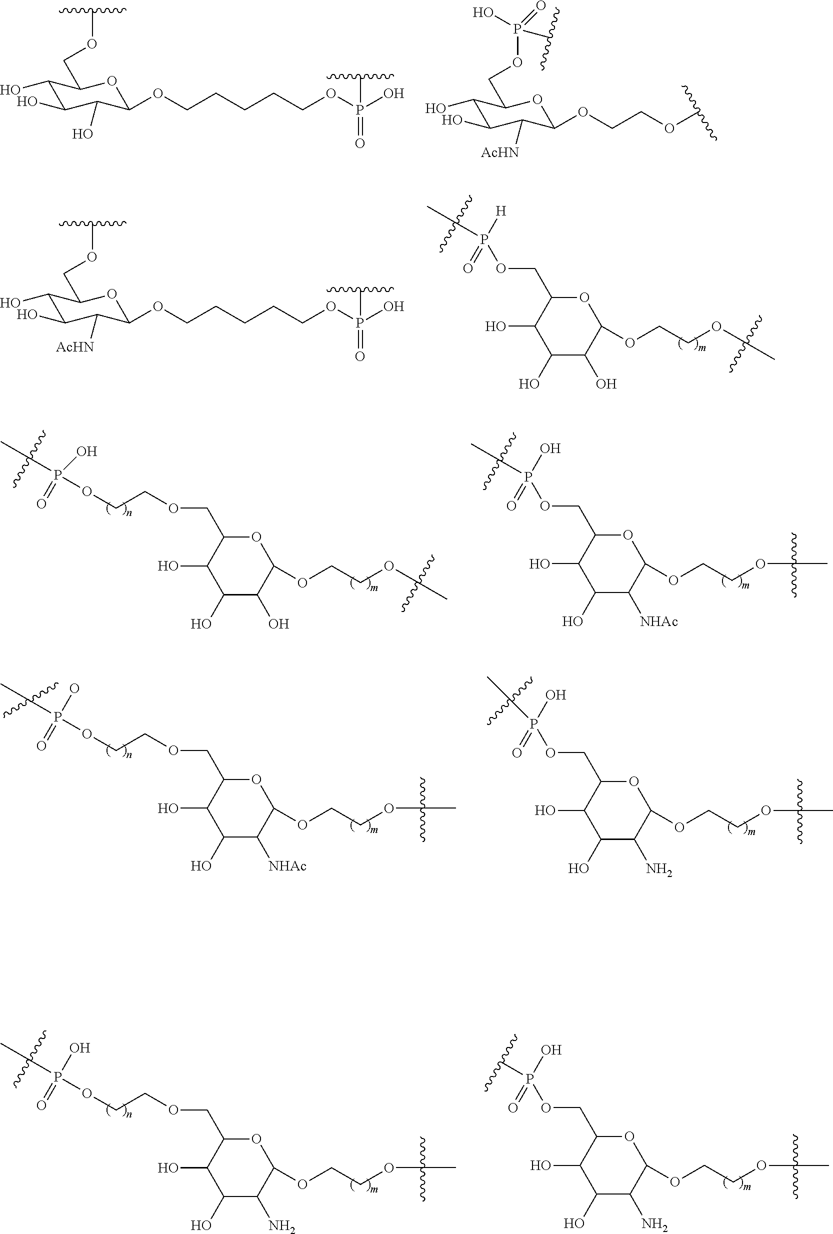

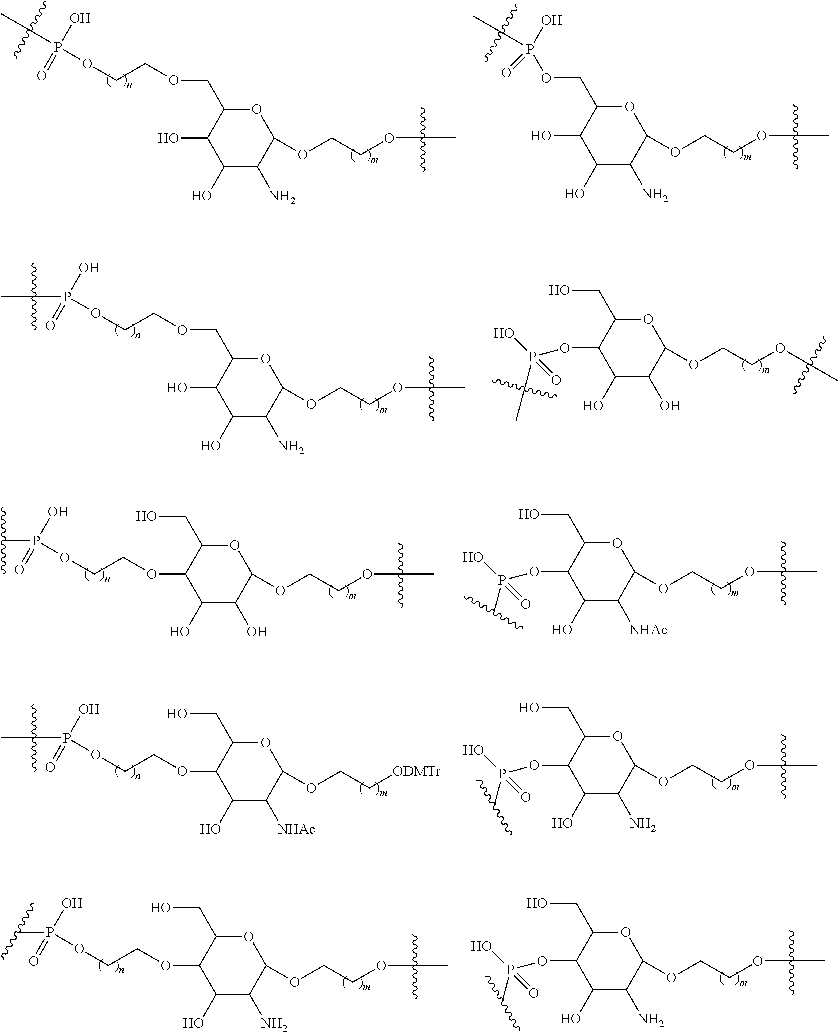

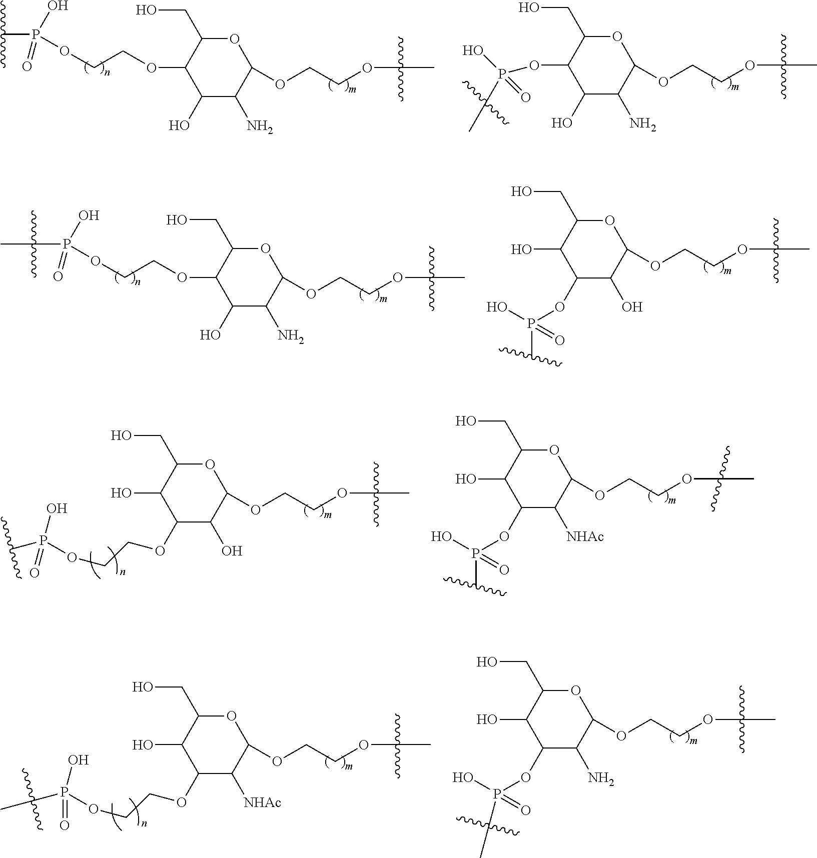

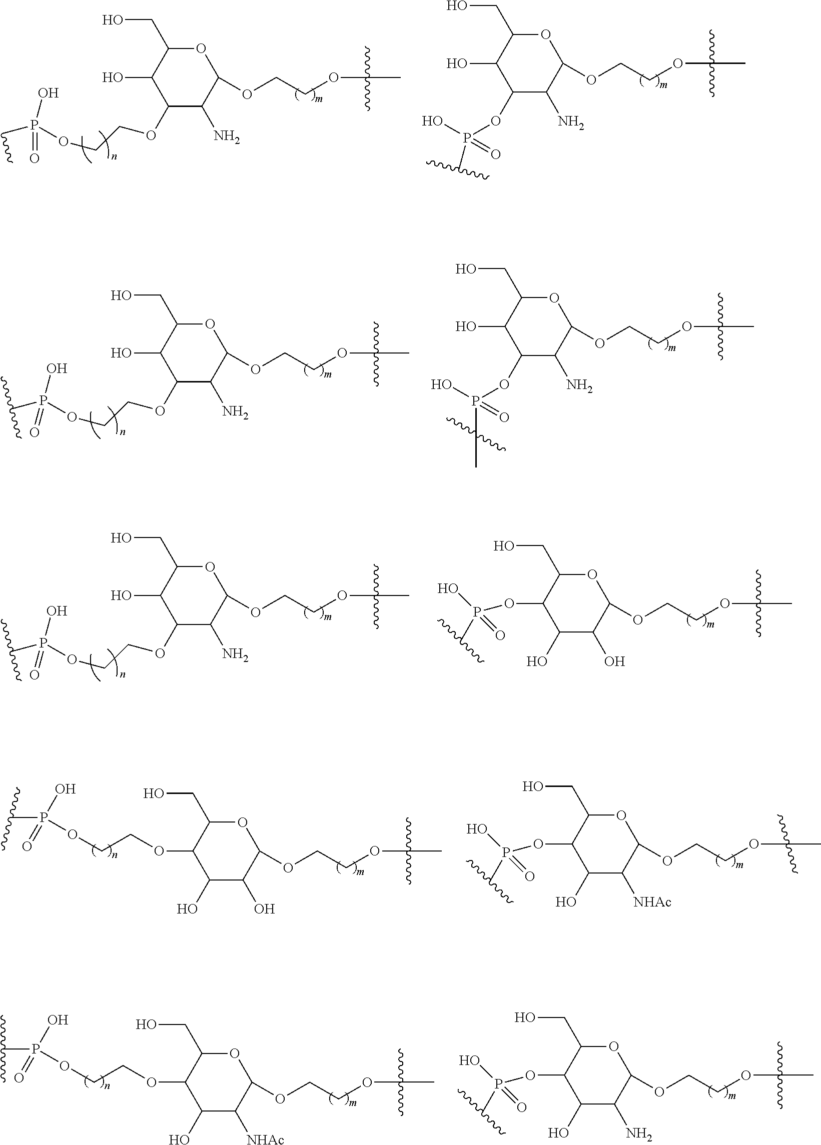

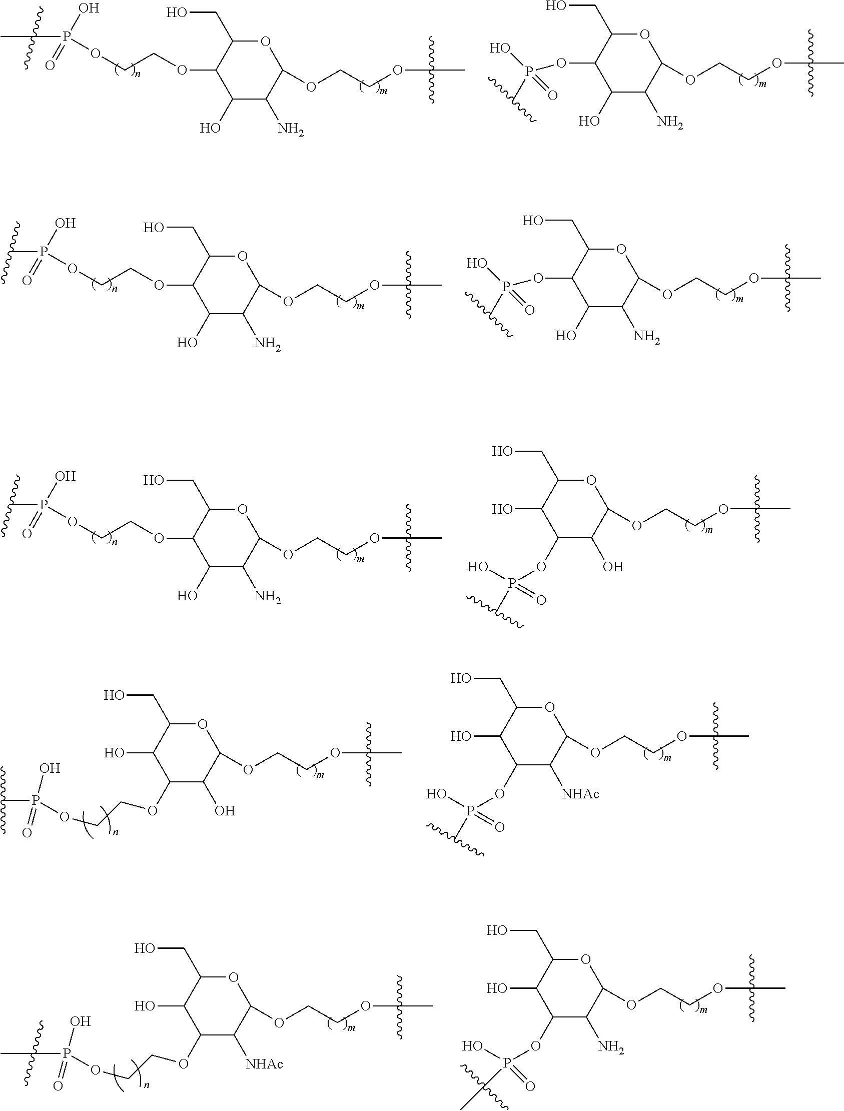

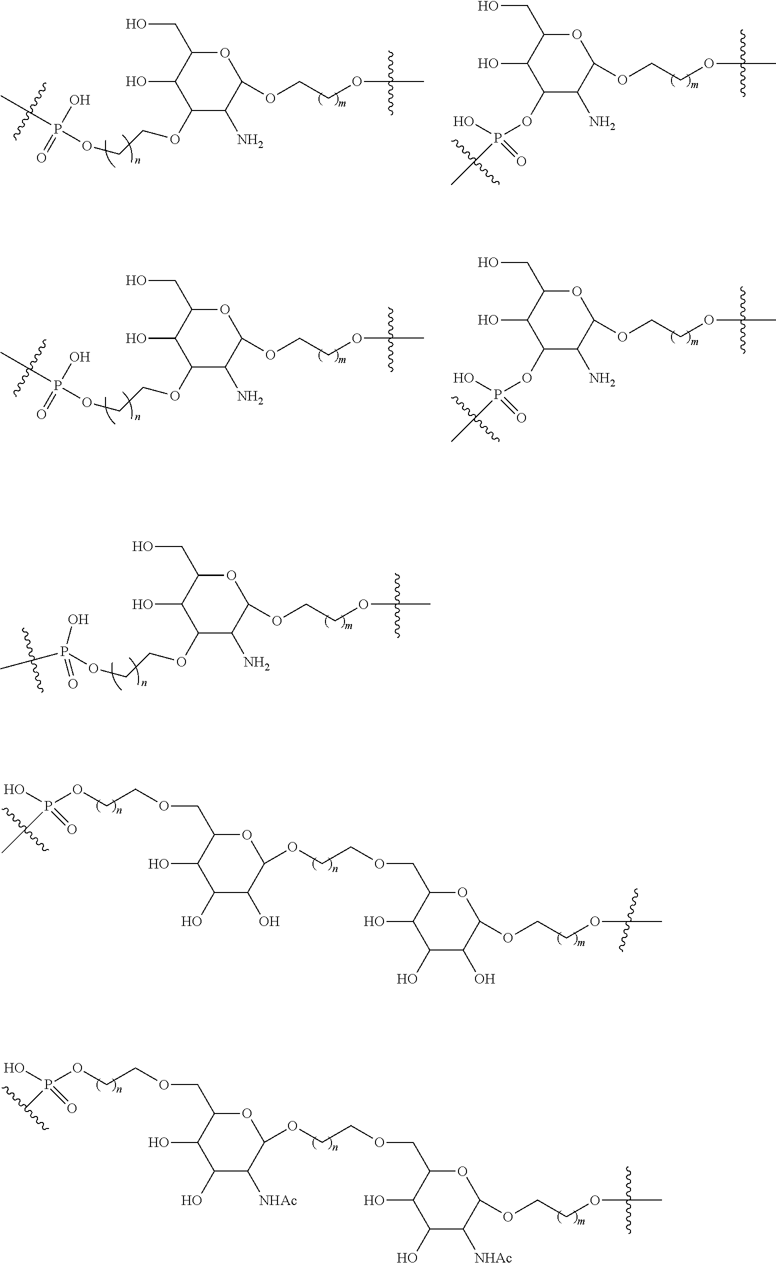

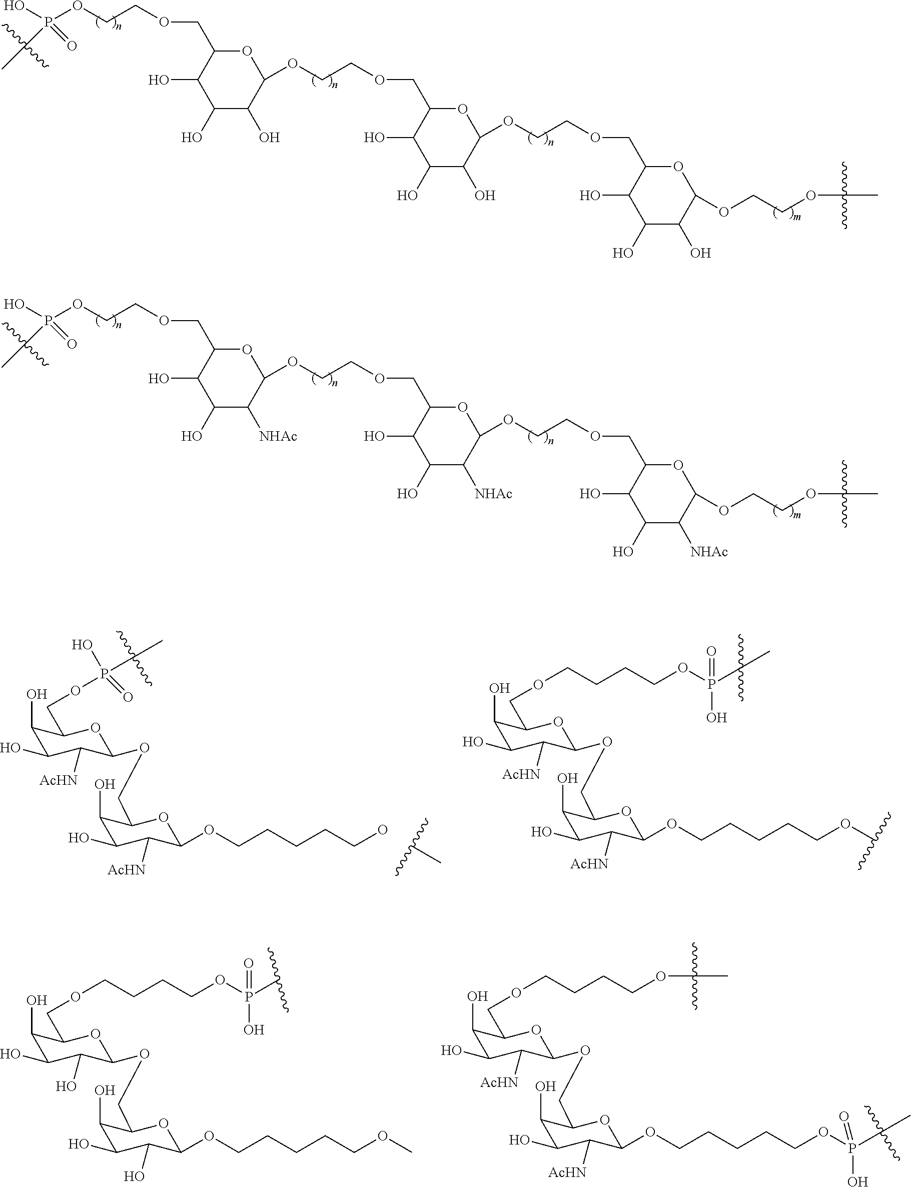

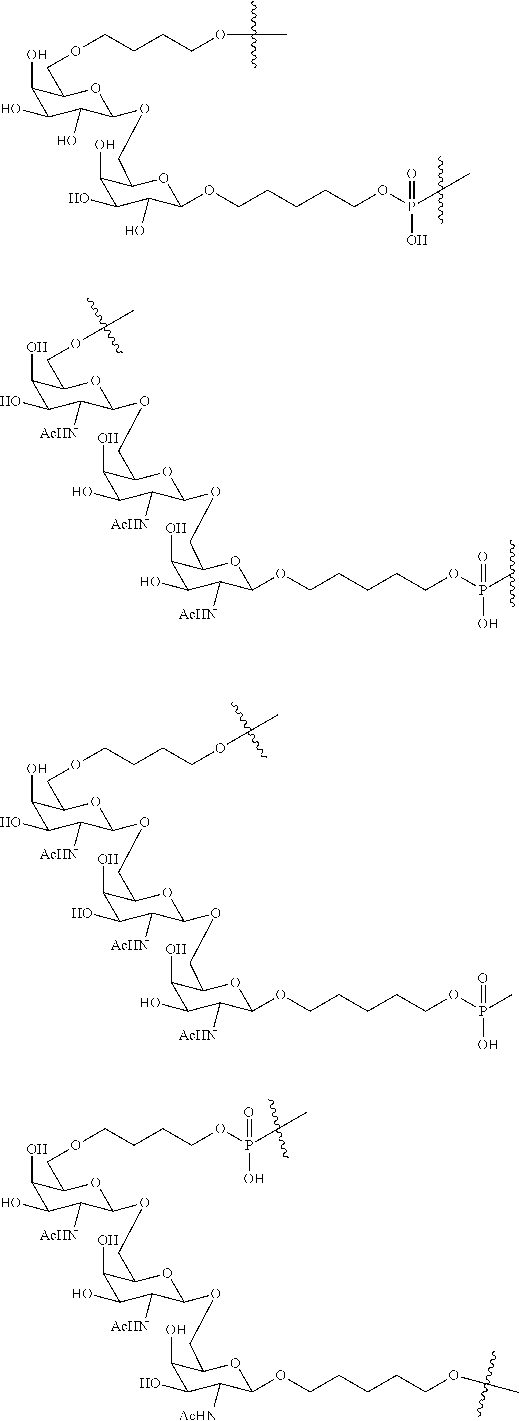

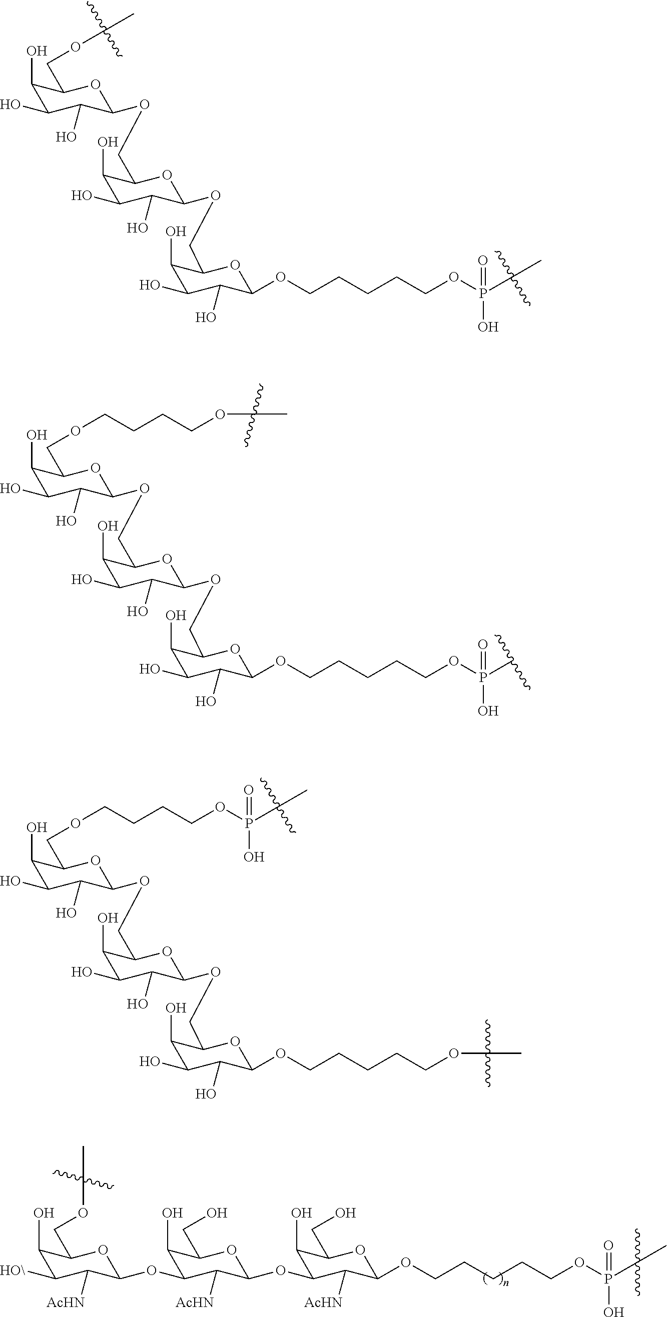

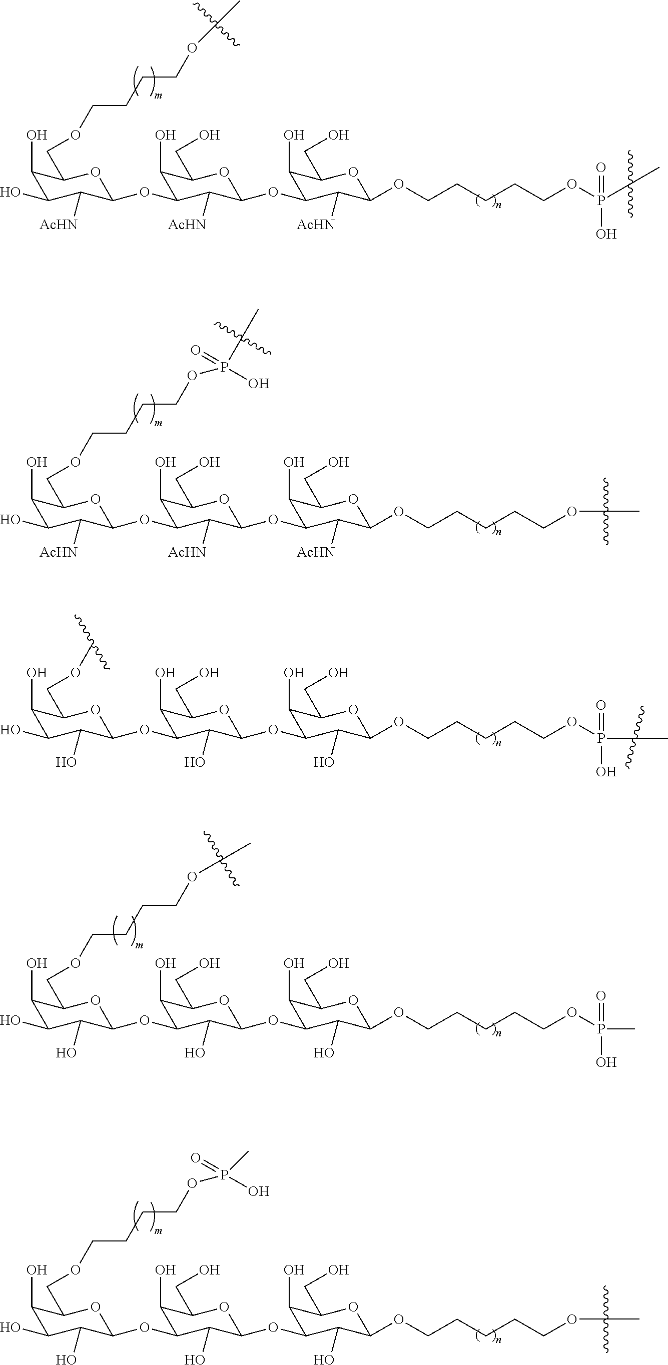

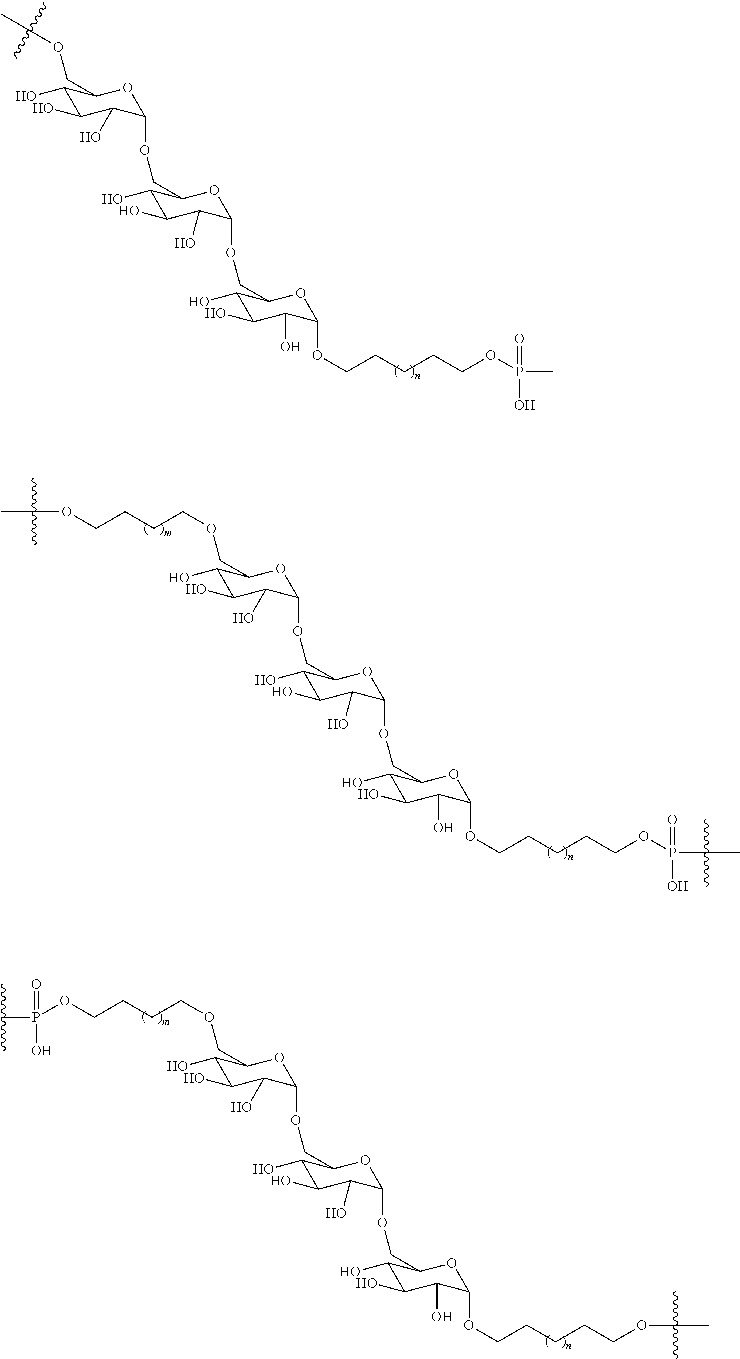

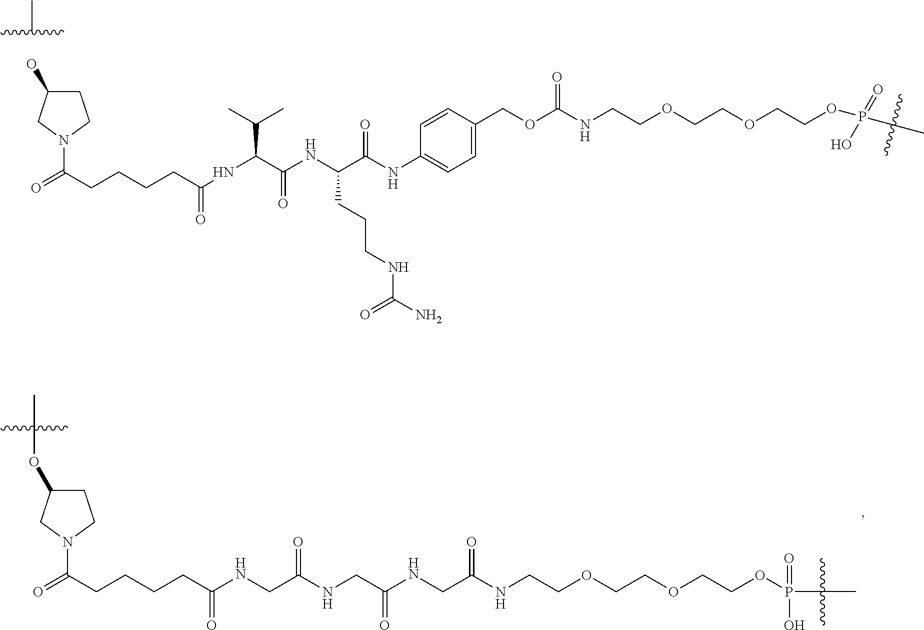

wherein n=1-12 and m=1-12;

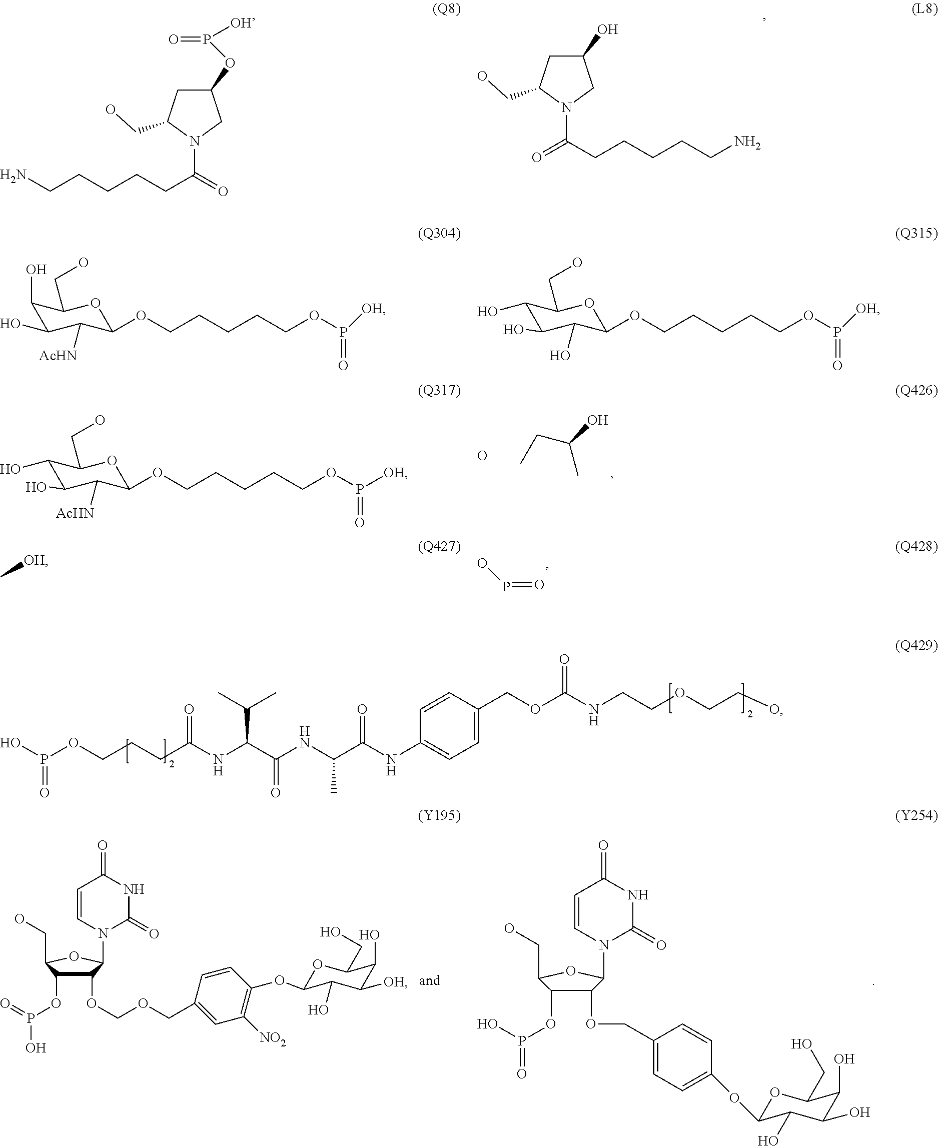

In certain embodiments, the nucleotide and/or non-nucleotide linkers are connected with the oligonucleotide strands through a phosphate diester linkage.

In some embodiments, the nucleotide and/or non-nucleotide linkers are connected with the oligonucleotide strands through a phosphate triester linkage.

In certain embodiments, the nucleotide and/or non-nucleotide linkers are connected with the oligonucleotide strands through a phosphate triester linkage, having the linkage phosphorus atom in either Rp configuration or Sp configuration.

In some embodiments, the nucleotide and/or non-nucleotide linkers are connected with the oligonucleotide strands through a phosphorothioate diester linkage.

In certain embodiments, the nucleotide and/or non-nucleotide linkers are connected with the oligonucleotide strands through a phosphorothioate diester linkage, having the linkage phosphorus atom in either Rp configuration or Sp configuration.

In some embodiments, the nucleotide and/or non-nucleotide linkers are connected with the oligonucleotide strands through a phosphoramidate diester linkage.

In certain embodiments, the nucleotide and/or non-nucleotide linkers are connected with the oligonucleotide strands through a phosphoramidate diester linkage, having the linkage phosphorus atom in either Rp configuration or Sp configuration.

In some embodiments, the nucleotide and/or non-nucleotide linkers are connected with the oligonucleotide strands through a disulfide linkage.

In certain embodiments, the antisense strand of at least one dsRNA is 23 nucleotides in length. Optionally, the antisense strands of each of the dsRNAs are 23 nucleotides in length. In some embodiments, the ultimate and penultimate nucleotides of the 3′-end of the antisense strand do not base pair with the sense strand oligonucleotide, optionally thereby forming a 3′-overhang with respect to the 5′-end of the corresponding sense strand dsRNA.

In another embodiment, the multi-targeted molecule, or an effector of the multi-targeted molecule, further includes a phosphate or phosphate mimic at the 5′-end of the antisense strand. Optionally, the phosphate mimic is a 5′-vinyl phosphonate (VP).

In some embodiments, the multi-targeted molecule includes a targeting ligand that targets a receptor which mediates delivery to a CNS tissue, e.g., a hydrophobic ligand. In certain embodiments, the targeting ligand is a C16 ligand.

In some embodiments, the multi-targeted molecule includes a targeting ligand that targets a brain tissue, e.g., striatum.

In certain embodiments, the tissue of the CNS of the subject is right hemisphere, left hemisphere, cerebellum, striatum, brainstem and/or spinal cord.

In one embodiment, the lipophilic moiety or targeting ligand is conjugated via a bio-cleavable linker that is DNA, RNA, disulfide, amide, functionalized monosaccharides or oligosaccharides of galactosamine, glucosamine, glucose, galactose, mannose, or a combination thereof.

In some embodiments, one or more lipophilic moiety is conjugated to the nucleic acid composition by a linker comprising a compound selected from the group consisting of an ether, a thioether, a urea, a carbonate, an amine, an amide, a maleimide-thioether, a disulfide, a phosphodiester, a sulfonamide linkage, a product of a click reaction, and a carbamate.

In some embodiments, one or more lipophilic moiety is conjugated to a location in the nucleic acid composition selected from the group consisting of a nucleobase, a sugar moiety, and an internucleosidic linkage.

In certain embodiments, the multi-targeted molecule is capable of inhibiting the activity or expression of the one or more distinct target RNAs in a tissue of the CNS of the subject by at least 20% each relative to an appropriate control. Optionally, the multi-targeted molecule is capable of inhibiting the activity or expression of the one or more distinct target RNAs in a tissue of the CNS of the subject by at least 25% each relative to an appropriate control. Optionally, the multi-targeted molecule is capable of inhibiting the activity or expression of the one or more distinct target RNAs in a tissue of the CNS of the subject by at least 30% each relative to an appropriate control. Optionally, the multi-targeted molecule is capable of inhibiting the activity or expression of the one or more distinct target RNAs in a tissue of the CNS of the subject by at least 35% each relative to an appropriate control. Optionally, the multi-targeted molecule is capable of inhibiting the activity or expression of the one or more distinct target RNAs in a tissue of the CNS of the subject by at least 40% each relative to an appropriate control. Optionally, the multi-targeted molecule is capable of inhibiting the activity or expression of the one or more distinct target RNAs in a tissue of the CNS of the subject by at least 45% each relative to an appropriate control. Optionally, the multi-targeted molecule is capable of inhibiting the activity or expression of the one or more distinct target RNAs in a tissue of the CNS of the subject by at least 50% each relative to an appropriate control.

In a related embodiment, the appropriate control is an untreated subject.

In some embodiments, the appropriate control is a reference value. Optionally, the reference value is a value obtained for the subject prior to administration of the multi-targeted molecule to the subject.

In certain embodiments, the multi-targeted molecule is formulated for intracerebroventricular (ICV) administration.

In some embodiments, the one or more distinct target RNAs are mRNAs. Optionally, the two or more distinct target RNAs are mRNAs.

In some embodiments, the one or more distinct target RNAs are transcripts of genes associated with a CNS disease or disorder. Optionally, the two or more distinct target RNAs are transcripts of genes associated with a CNS disease or disorder.

In some embodiments, the 3′ end of the sense strand of the multi-targeted molecule is protected via an end cap which is a cyclic group having an amine, the cyclic group being pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

In some embodiments, the multi-targeted molecule further includes: a terminal, chiral modification occurring at the first internucleotide linkage at the 3′ end of the antisense strand of one or multiple dsRNAs, having the linkage phosphorus atom in Sp configuration; a terminal, chiral modification occurring at the first internucleotide linkage at the 5′ end of the antisense strand of one or multiple dsRNAs, having the linkage phosphorus atom in Rp configuration; or a terminal, chiral modification occurring at the first internucleotide linkage at the 5′ end of the sense strand of one or multiple dsRNAs, having the linkage phosphorus atom in either Rp configuration or Sp configuration.

In some embodiments, the multi-targeted molecule further includes: a terminal, chiral modification occurring at the first and second internucleotide linkages at the 3′ end of the antisense strand of one or multiple dsRNAs, having the linkage phosphorus atom in Sp configuration; a terminal, chiral modification occurring at the first internucleotide linkage at the 5′ end of the antisense strand of one or multiple dsRNAs, having the linkage phosphorus atom in Rp configuration; or a terminal, chiral modification occurring at the first internucleotide linkage at the 5′ end of the sense strand of one or multiple dsRNAs, having the linkage phosphorus atom in either Rp or Sp configuration.

In certain embodiments, the multi-targeted molecule further includes: a terminal, chiral modification occurring at the first, second and third internucleotide linkages at the 3′ end of the antisense strand of one or multiple dsRNAs, having the linkage phosphorus atom in Sp configuration; a terminal, chiral modification occurring at the first internucleotide linkage at the 5′ end of the antisense strand of one or multiple dsRNAs, having the linkage phosphorus atom in Rp configuration; or a terminal, chiral modification occurring at the first internucleotide linkage at the 5′ end of the sense strand of one or multiple dsRNAs, having the linkage phosphorus atom in either Rp or Sp configuration.

In some embodiments, the multi-targeted molecule further includes: a terminal, chiral modification occurring at the first, and second internucleotide linkages at the 3′ end of the antisense strand of one or multiple dsRNAs, having the linkage phosphorus atom in Sp configuration; a terminal, chiral modification occurring at the third internucleotide linkages at the 3′ end of the antisense strand of one or multiple dsRNAs, having the linkage phosphorus atom in Rp configuration; a terminal, chiral modification occurring at the first internucleotide linkage at the 5′ end of the antisense strand of one or multiple dsRNAs, having the linkage phosphorus atom in Rp configuration; or a terminal, chiral modification occurring at the first internucleotide linkage at the 5′ end of the sense strand of one or multiple dsRNAs, having the linkage phosphorus atom in either Rp or Sp configuration.

In some embodiments, the multi-targeted molecule further includes: a terminal, chiral modification occurring at the first, and second internucleotide linkages at the 3′ end of the antisense strand of one or multiple dsRNAs, having the linkage phosphorus atom in Sp configuration; a terminal, chiral modification occurring at the first, and second internucleotide linkages at the 5′ end of the antisense strand of one or multiple dsRNAs, having the linkage phosphorus atom in Rp configuration; or a terminal, chiral modification occurring at the first internucleotide linkage at the 5′ end of the sense strand of one or multiple dsRNAs, having the linkage phosphorus atom in either Rp or Sp configuration.

In certain embodiments, the nucleic acid composition includes three or more linked dsRNAs, single-stranded nucleic acid agents, or combinations thereof.

Another aspect of the instant disclosure provides a method for modulating in the central nervous system (CNS) of a subject one or more target RNAs having one or more distinct target RNA sequences, the method involving contacting the CNS cell of the subject with a multi-targeted molecule having at least two nucleic acid-based effector molecules (where at least one is a dsRNA), wherein the effector molecules are connected together by a linker and do not overlap with each other, wherein each of the at least two effector molecules that is a dsRNA includes at least one conjugated lipophilic moiety, and wherein the multi-targeted molecule inhibits the activity or expression of the one or more target RNAs in the CNS of the subject by at least 15% each relative to an appropriate control.

A further aspect of the instant disclosure provides a method for treating or preventing a disease or disorder of the CNS in a subject having or at risk of developing the disease or disorder of the CNS, the method including administering to the CNS of the subject a multi-targeted molecule as disclosed herein, thereby treating the subject.

Exemplary diseases or disorders of the CNS that can be treated or prevented using the compositions or methods of the instant disclosure include, without limitation, neurodegenerative disorders (e.g., Parkinson's Disease (PD), Alzheimer's disease, early onset familial Alzheimer's disease (EOFAD), cerebral amyloid angiopathy (CAA), Spinal Muscular Atrophy (SMA), Angelman Syndrome, ataxias/neurodegenerative disorders of the nervous system (e.g., Friedreich's Ataxia), Huntington's disease (Huntington chorea), multiple sclerosis, amyotrophic lateral sclerosis (ALS)), depression, Down's syndrome, psychosis, schizophrenia, Creutzfeldt-Jakob disease, multiple system atrophy, Lewy body dementia (LBD), pure autonomic failure (PAF), Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, vascular disorders (e.g., stroke, transient ischemic attack (TIA), subarachnoid hemorrhage, subdural hemorrhage and hematoma, and extradural hemorrhage), infections (e.g., meningitis, encephalitis, polio, epidural abscess), structural disorders (e.g., brain or spinal cord injury, Bell's palsy, cervical spondylosis, carpal tunnel syndrome, brain or spinal cord tumors, peripheral neuropathy, Guillain-Barre syndrome) and functional disorders (e.g., headache, epilepsy, dizziness, neuralgia). Other diseases or disorders of the CNS that can be treated or prevented using the compositions or methods of the instant disclosure include, without limitation, Spinal Muscular Atrophy (SMA), Angelman Syndrome, and ataxia's/neurodegenerative disorders of the nervous system (e.g., Friedreich's Ataxia).

In one embodiment, treating involves amelioration of at least on sign or symptom of the disease or disorder.

In certain embodiments, treating includes prevention of progression of the disease or disorder.

One aspect of the invention provides a pharmaceutical composition for inhibiting expression of one or more target genes having one or more distinct target RNA sequences, optionally wherein at least one target gene is associated with a CNS disease or disorder, the pharmaceutical composition formulated for administration to the CNS of a subject and including a multi-targeted molecule of the instant disclosure and a pharmaceutically acceptable carrier.

In some embodiments, the instant disclosure provides an injectate formulated for CNS delivery that includes a pharmaceutical composition of the instant disclosure.

An additional aspect of the disclosure provides a method of inhibiting expression of a target gene associated with a CNS disease or disorder in a CNS cell, the method involving: (a) contacting the cell with a multi-targeted molecule of the instant disclosure or a pharmaceutical composition of the instant disclosure; and (b) maintaining the cell produced in step (a) for a time sufficient to obtain degradation of the mRNA transcript of the target gene associated with a CNS disease or disorder, thereby inhibiting expression of the target gene associated with a CNS disease or disorder in the cell.

In one embodiment, the cell is within a subject. Optionally, the subject is a human.

In certain embodiments, the subject is a mammal. Optionally, the subject is a rhesus monkey, a cynomolgous monkey, a mouse, or a rat.

In some embodiments, the expression of each target gene associated with a CNS disease or disorder is inhibited by at least 15%, optionally by at least 20%, optionally by at least 25%, optionally by at least 30%, optionally by at least 35%, optionally by at least 40%, optionally by at least 45%, optionally by at least 50%.

In certain embodiments, the subject meets at least one diagnostic criterion for a CNS disease or disorder.

In certain embodiments, the human subject has been diagnosed with or suffers from a disease selected from the group consisting of a neurodegenerative disorders (e.g., Parkinson's Disease (PD), Alzheimer's disease, early onset familial Alzheimer's disease (EOFAD), cerebral amyloid angiopathy (CAA), Spinal Muscular Atrophy (SMA), Angelman Syndrome, ataxias/neurodegenerative disorders of the nervous system (e.g., Friedreich's Ataxia), Huntington's disease (Huntington chorea), multiple sclerosis, amyotrophic lateral sclerosis (ALS)), depression, Down's syndrome, psychosis, schizophrenia, Creutzfeldt-Jakob disease, multiple system atrophy, Lewy body dementia (LBD), pure autonomic failure (PAF), Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, a vascular disorder (e.g., stroke, transient ischemic attack (TIA), subarachnoid hemorrhage, subdural hemorrhage and hematoma, and extradural hemorrhage), an infection (e.g., meningitis, encephalitis, polio, epidural abscess), a structural disorder (e.g., brain or spinal cord injury, Bell's palsy, cervical spondylosis, carpal tunnel syndrome, brain or spinal cord tumors, peripheral neuropathy, Guillain-Barre syndrome) and a functional disorder (e.g., headache, epilepsy, dizziness, neuralgia).

In some embodiments, the step of contacting involves administering an intrathecal or intracerebroventricular (ICV) injectate to the subject.

In certain embodiments, the method further involves administering an additional therapeutic agent or therapy to the subject. Exemplary additional therapeutics and treatments include, for example, sedatives, antidepressants, clonazepam, sodium valproate, opiates, antiepileptic drugs, cholinesterase inhibitors, memantine, benzodiazepines, levodopa, COMT inhibitors (e.g., tolcapone and entacapone), dopamine agonists (e.g., bromocriptine, pergolide, pramipexole, ropinirole, piribedil, cabergoline, apomorphine and lisuride), MAO-B inhibitors (e.g., safinamide, selegiline and rasagiline), surgery, amantadine, an anticholinergic, modafinil, pimavanserin, doxepin, rasagline, an antipsychotic, an atypical antipsychotic (e.g., amisulpride, olanzapine, risperidone, and clozapine), riluzole, edaravone, deep brain stimulation, non-invasive ventilation (NIV), invasive ventilation physical therapy, occupational therapy, speech therapy, dietary changes and swallowing technique a feeding tube, a PEG tube, probiotics, and psychological therapy.

In certain embodiments, the multi-targeted molecule of the instant disclosure is administered at a dose of about 0.01 mg/kg to about 50 mg/kg.

In some embodiments, the multi-targeted molecule of the instant disclosure is administered to the subject intrathecally.

In one embodiment, the method reduces the expression of the target gene associated with a CNS disease or disorder in a brain (e.g., striatum) or spine tissue. Optionally, the brain or spine tissue is striatum, cortex, cerebellum, cervical spine, lumbar spine, or thoracic spine.

In some embodiments, the multi-targeted molecule further includes at least one phosphorothioate or methylphosphonate internucleotide linkage. In a related embodiment, the phosphorothioate or methylphosphonate internucleotide linkage is at the 3′-terminus of one strand, or is optionally at the 3′-end of at least one strand of each dsRNA of the multi-targeted molecule. Optionally, the strand is the antisense strand. In another embodiment, the strand is the sense strand. In a related embodiment, the phosphorothioate or methylphosphonate internucleotide linkage is at the 5′-terminus of one strand, or is optionally at the 5′-end of at least one strand of each dsRNA of the multi-targeted molecule. Optionally, the strand is the antisense strand. In another embodiment, the strand is the sense strand.

In another embodiment, the phosphorothioate or methylphosphonate internucleotide linkage is at both the 5′- and 3′-terminus of one strand, or is optionally at both the 5′- and 3′-end of at least one strand of each dsRNA of the multi-targeted molecule. Optionally, the strand is the antisense strand. In another embodiment, the strand is the sense strand.

In an additional embodiment, the base pair at the 1 position of the 5′-end of the antisense strand of the multi-targeted molecule, or of an dsRNA of the multi-targeted molecule, is an A:U base pair.

An additional aspect of the instant disclosure provides a multi-targeted molecule for inhibiting expression of a target gene, wherein one or more dsRNA that is targeted to a target gene (each dsRNA of the multi-targeted molecule being targeted to different parts of the same gene or to different genes) includes a sense strand and an antisense strand forming a double stranded region, wherein the sense strand includes at least 15 contiguous nucleotides differing by no more than 3 nucleotides (i.e., differing by 3, 2, 1, or 0 nucleotides) from any one of the nucleotide sequences of the target gene of the dsRNA, or a nucleotide sequence having at least 90% nucleotide sequence identity, e.g. 90, 91, 92, 93, 94, 95, 96, 97, 98, 99 or 100% identity, to the entire nucleotide sequence of the target gene of the dsRNA, and the antisense strand includes at least 15 contiguous nucleotides differing by no more than 3 nucleotides (i.e., differing by 3, 2, 1, or 0 nucleotides) from the complement of any one of the nucleotide sequences of the target gene of the dsRNA, or a nucleotide sequence having at least 90% nucleotide sequence identity, e.g. 90, 91, 92, 93, 94, 95, 96, 97, 98, 99 or 100% identity, to the complement of the entire nucleotide sequence of the target gene of the dsRNA; wherein a substitution of a uracil for any thymine in the sequences of the target gene of the dsRNA (when comparing aligned sequences) does not count as a difference that contributes to the differing by no more than 3 nucleotides from any one of the complement nucleotide sequences provided in the target nucleotide sequence(s) of the dsRNA, optionally wherein substantially all of the nucleotides of the sense strand of one or multiple dsRNAs include a modification that is a 2′-O-methyl modification, a GNA or a 2′-fluoro modification, optionally wherein the sense strand of one or multiple dsRNAs includes two phosphorothioate internucleotide linkages at the 5′-terminus, optionally wherein substantially all of the nucleotides of the antisense strand of one or multiple dsRNAs include a modification selected from the group consisting of a 2′-O-methyl modification and a 2′-fluoro modification, optionally wherein the antisense strand of one or multiple dsRNAs includes two phosphorothioate internucleotide linkages at the 5′-terminus and two phosphorothioate internucleotide linkages at the 3′-terminus, and optionally wherein the sense strand of one or multiple dsRNAs is conjugated to one or more lipophilic, e.g., C16, ligands. In certain embodiments, the sense strand of one or multiple dsRNAs includes at least one 3′-terminal deoxythimidine nucleotide (dT), and optionally the antisense strand of one or multiple dsRNAs includes at least one 3′-terminal deoxythimidine nucleotide (dT).

In one embodiment, all of the nucleotides of the sense strand of one or multiple dsRNAs of the multi-targeted molecule are modified nucleotides, and optionally all of the nucleotides of the antisense strand of one or multiple dsRNAs of the multi-targeted molecule are modified nucleotides.

In another embodiment, each strand of one or multiple dsRNAs of the multi-targeted molecule each has 19-30 nucleotides.

In certain embodiments, the antisense strand of one or multiple dsRNAs of the multi-targeted molecule includes at least one thermally destabilizing modification of the duplex within the first 9 nucleotide positions of the 5′ region or a precursor thereof. Optionally, the thermally destabilizing modification of the duplex is one or more of

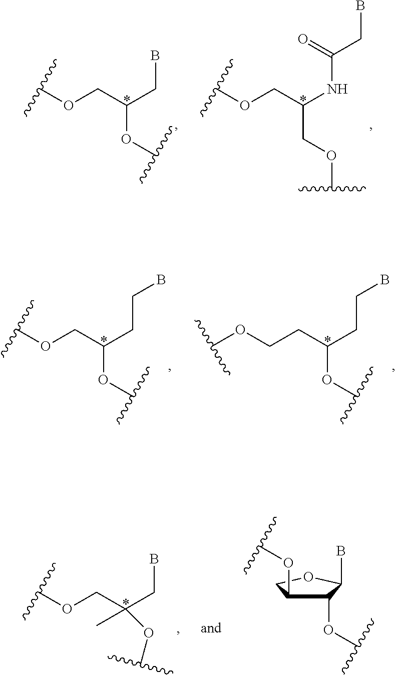

wherein B is nucleobase.

Another aspect of the instant disclosure provides a cell containing a multi-targeted molecule of the instant disclosure. Optionally, the cell is a cell of a CNS tissue of a subject.

An additional aspect of the instant disclosure provides a pharmaceutical composition for inhibiting expression of a target gene that includes a multi-targeted molecule of the instant disclosure.

In one embodiment, the multi-targeted molecule is administered in an unbuffered solution. Optionally, the unbuffered solution is saline or water.

In another embodiment, the multi-targeted molecule is administered with a buffer solution. Optionally, the buffer solution includes acetate, citrate, prolamine, carbonate, or phosphate or any combination thereof. In another embodiment, the buffer solution is phosphate buffered saline (PBS).

Another aspect of the disclosure provides a pharmaceutical composition that includes a multi-targeted molecule of the instant disclosure and a lipid formulation.

In one embodiment, the lipid formulation includes a lipid nanoparticle (LNP).

Another aspect of the instant disclosure provides a kit for performing a method of the instant disclosure, the kit including: a) a multi-targeted molecule of the instant disclosure, and b) instructions for use, and c) optionally, a device for administering the multi-targeted molecule to the subject.

An additional aspect of the instant disclosure provides a multi-targeted molecule that includes one or more of the following modifications, optionally within each dsRNA: a 2′-O-methyl modified nucleotide, a 2′-fluoro modified nucleotide, a 2′-alkyl-modified nucleotide, a nucleotide comprising a glycol nucleic acid (GNA), a phosphorothioate (PS) and a vinyl phosphonate (VP). Optionally, the RNAi agent includes at least one of each of the following modifications: a 2′-O-methyl modified nucleotide, a 2′-fluoro modified nucleotide, a 2′-alkyl-modified nucleotide, a nucleotide comprising a glycol nucleic acid (GNA), a phosphorothioate and a vinyl phosphonate (VP).

In another embodiment, the multi-targeted molecule or a dsRNA of the multi-targeted molecule includes four or more PS modifications, optionally six to sixteen PS modifications, optionally eight to fourteen PS modifications, optionally ten to twelve PS modifications, optionally six in a dsRNA, optionally twelve in a multi-targeted molecule.

In an additional embodiment, the sense strand and the antisense strand of each dsRNA of the multi-targeted molecule possesses a 5′-terminus and a 3′-terminus (with linkers excluded from consideration), and one or multiple dsRNAs of the multi-targeted molecule includes six PS modifications positioned at each of the penultimate and ultimate internucleotide linkages of the following: 5′-termini and 3′-termini of the sense strands of each dsRNA of the multi-targeted molecule and 3′-termini of the antisense strands of each dsRNA of the multi-targeted molecule.

In a further embodiment, the sense strand and the antisense strand of each dsRNA of the multi-targeted molecule possesses a 5′-terminus and a 3′-terminus (with linkers excluded from consideration), and one or multiple dsRNAs of the multi-targeted molecule includes eight PS modifications positioned at each of the penultimate and ultimate internucleotide linkages from the respective 3′- and 5′-termini of each of the sense and antisense strands of the multi-targeted molecule.

In another embodiment, each of the sense strand and the antisense strand of each dsRNA of the multi-targeted molecule possesses a 5′-terminus and a 3′-terminus (with linkers excluded from consideration), and one or multiple dsRNAs of the multi-targeted molecule includes only one nucleotide including a GNA. Optionally, the nucleotide including a GNA is positioned on the antisense strand at the seventh nucleobase residue from the 5′-terminus of the antisense strand.

In an additional embodiment, each of the sense strand and the antisense strand of each dsRNA of the multi-targeted molecule possesses a 5′-terminus and a 3′-terminus (with linkers excluded from consideration), and one or multiple dsRNAs of the multi-targeted molecule includes one to four 2′-alkyl-modified nucleotides. Optionally, the 2′-alkyl-modified nucleotide is a 2′-C₁₆-modified nucleotide. Optionally, each of the one or multiple dsRNAs of the multi-targeted molecule includes a single 2′-alkyl, e.g., C₁₆-modified nucleotide. Optionally, the single 2′-alkyl, e.g., C₁₆-modified nucleotide is located on the sense strand at the sixth nucleobase position from the 5′-terminus of the sense strand.

In another embodiment, each of the sense strand and the antisense strand of each dsRNA of the multi-targeted molecule possesses a 5′-terminus and a 3′-terminus (with linkers excluded from consideration), and one or multiple dsRNAs of the multi-targeted molecule includes two or more 2′-fluoro modified nucleotides. Optionally, each of the sense strand and the antisense strand of one or multiple dsRNAs of the multi-targeted molecule includes two or more 2′-fluoro modified nucleotides. Optionally, the 2′-fluoro modified nucleotides are located on the sense strand at nucleobase positions 7, 9, 10 and 11 from the 5′-terminus of the sense strand and on the antisense strand at nucleobase positions 2, 14 and 16 from the 5′-terminus of the antisense strand. In certain embodiments, the antisense strand of each dsRNA further includes 2′-fluoro modified nucleotides at one or more of nucleobase positions 6, 8 and 9 from the 5′-terminus of the antisense strand. In a related embodiment, the 2′-fluoro modified nucleotides are located on the sense strand at nucleobase positions 7, 9, 10 and 11 from the 5′-terminus of the sense strand and on the antisense strand at nucleobase positions 2, 6, 8, 9, 14 and 16 from the 5′-terminus of the antisense strand.

In an additional embodiment, each of the sense strand and the antisense strand of each dsRNA of the multi-targeted molecule possesses a 5′-terminus and a 3′-terminus (with linkers excluded from consideration), and one or multiple dsRNAs of the multi-targeted molecule includes one or more VP modifications. Optionally, one or multiple dsRNAs of the multi-targeted molecule includes a single VP modification at the 5′-terminus of the antisense strand.

In another embodiment, each of the sense strand and the antisense strand of each dsRNA of the multi-targeted molecule possesses a 5′-terminus and a 3′-terminus (with linkers excluded from consideration), and one or multiple dsRNAs of the multi-targeted molecule includes two or more 2′-O-methyl modified nucleotides. Optionally, one or multiple dsRNAs of the multi-targeted molecule includes 2′-O-methyl modified nucleotides at all nucleobase locations not modified by a 2′-fluoro, a 2′-alkyl or a glycol nucleic acid (GNA). Optionally, the two or more 2′-O-methyl modified nucleotides are located on the sense strand at positions 1, 2, 3, 4, 5, 8, 12, 13, 14, 15, 16, 17, 18, 19, 20 and 21 from the 5′-terminus of the sense strand and on the antisense strand at positions 1, 3, 4, 5, 6, 8, 9, 10, 11, 12, 13, 15, 17, 18, 19, 20, 21, 22 and 23 from the 5′-terminus of the antisense strand. Alternatively, the two or more 2′-O-methyl modified nucleotides are located on the sense strand at positions 1, 2, 3, 4, 5, 8, 12, 13, 14, 15, 16, 17, 18, 19, 20 and 21 from the 5′-terminus of the sense strand and on the antisense strand at positions 1, 3, 4, 5, 7, 10, 11, 12, 13, 15, 17, 18, 19, 20, 21, 22 and 23 from the 5′-terminus of the antisense strand.

Another aspect of the invention provides a cell of a tissue of the CNS of a subject comprising a nucleic acid composition comprising at least a first dsRNA molecule and a single-stranded nucleic acid agent or second dsRNA molecule, wherein the first dsRNA molecule and the single-stranded nucleic acid agent or second dsRNA molecule are connected together by a linker and do not overlap with each other, wherein each of the first dsRNA molecule and the single-stranded nucleic acid agent or second dsRNA molecule comprises at least one conjugated lipophilic moiety, and wherein said nucleic acid composition inhibits the activity or expression of one or more distinct target RNAs in the cell or tissue of the CNS of the subject by at least 15% each relative to an appropriate control.

In some embodiments, the cell is of a type is selected from the group consisting of a neuron, an oligodendrocyte, a microglia cell and an astrocyte.

Another aspect of the invention provides a multi-targeted molecule, for modulation of two or more distinct target RNAs in the central nervous system (CNS) of a subject, according to the formula:

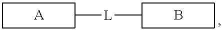

wherein A is first double-stranded oligonucleotide (e.g., dsRNA (dsRNA)) molecule; B is a second double-stranded oligonucleotide (e.g., dsRNA) molecule; and L is a linker, wherein A and B do not overlap with each other, and each of A and B, independently, include at least one conjugated lipophilic moiety, and wherein the multi-targeted molecule is capable of inhibiting the activity or expression of the two or more distinct target RNAs in a tissue of the CNS of the subject by at least 15% each, relative to an appropriate control.

In certain embodiments, A and B are, respectively, first and second double-stranded RNA molecules (dsRNA).

In one embodiment, A is according to the formula,

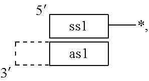

wherein ss1 is the sense strand of the first dsRNA molecule; as1 is the antisense strand of the first dsRNA molecule; * represents the bond between ss1 and L; and the dotted box indicates an optional 3′-overhang region of as1.

In another embodiment, A is according to the formula,

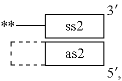

wherein ss2 is the sense strand of the second dsRNA molecule; as2 is the antisense strand of the second dsRNA molecule; ** represents the bond between ss2 and L; and the dotted box indicates an optional 3′-overhang region of as2.

In a related embodiment, L is represented by -(nt1)(nt2)(nt3)-, wherein each of nt1, nt2, and nt3 are independently a nucleotide or modified nucleotide, and wherein nt1 is bonded to the first dsRNA molecule and nt3 is bonded to the second dsRNA molecule.

In another embodiment, the multi-targeted molecule has the formula:

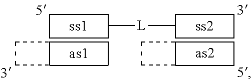

wherein ss1 is the sense strand of the first dsRNA molecule; as1 is the antisense strand of the first dsRNA molecule; ss2 is the sense strand of the second dsRNA molecule; as2 is the antisense strand of the second dsRNA molecule; and the dotted boxes indicate optional 3′-overhang regions of as1 and as2, respectively.

In some embodiments, L is represented by -(nt1)(nt2)(nt3)-, wherein each of nt1, nt2, and nt3 are independently a nucleotide or modified nucleotide, and wherein nt1 is bonded to the first dsRNA molecule and nt3 is bonded to the second dsRNA molecule.

In some embodiments, L is represented by -(nt1)(nt2)(nt3)-, wherein each of nt1, nt2, and nt3 are independently a nucleotide or a modified nucleotide, and wherein nt1 is bonded to ss1 and nt3 is bonded to ss2. In a related embodiment, nt1, nt2, and nt3 are each independently selected from the following: A, T, U, G, C, dA, dT, dU, dG, dC, a, t, u, g, c, Af, Tf, Uf, Gf, and Cf, as defined in Table 1.

In another embodiment, nt1, nt2, and nt3 are each independently A, T, U, G or C, as defined in Table 1.

In a further embodiment, nt1, nt2, and nt3 are each independently dA, dT, dU, dG or dC, as defined in Table 1.

In some embodiments, nt1, nt2, and nt3 are each independently a, t, u, g or c, as defined in Table 1.

In certain embodiments, nt1, nt2, and nt3 are each independently Af, Tf, Uf, Gf or Cf, as defined in Table 1.

In some embodiments, nt1, nt2, and nt3 are collectively one of the following: dTdTdT, UUU, uuu, and UfUfUf, as defined in Table 1.

In some embodiments, as1 and as2 each comprise an independent two nucleotide 3′-overhang. In a related embodiment, the two nucleotide 3′-overhang of as2 is complementary to the linker. In certain embodiments, the two nucleotide 3′-overhang of as2 has one mismatch to the linker. In another embodiment, the two nucleotide 3′-overhang of as2 has two mismatches to the linker.

In some embodiments, each lipophilic moiety is a hexadecyl group.

In certain embodiments, one or more non-terminal nucleotide positions of the first sense strand (e.g., position 6) and one or more non-terminal nucleotide positions of the second sense strand independently have the following structure:

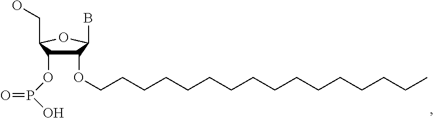

wherein B is a nucleotide base or a nucleotide base analog, optionally wherein B is adenine, guanine, cytosine, thymine or uracil, wherein the n-hexadecyl chain is the lipophilic moiety.

In some embodiments, the one or more non-terminal nucleotide positions of the first sense strand are selected from the group consisting of positions 2-8 and 13-20 of the first sense strand, optionally selected from the group consisting of positions 4-8 and 13-18, optionally wherein the non-terminal nucleotide positions are positions 4, 6, 7 and 8, or are positions 5, 6, 7, 15, and 17 of the first sense strand; and the one or more non-terminal nucleotide positions of the second sense strand are selected from the group consisting of positions 2-8 and 13-20 of the first sense strand, optionally selected from the group consisting of positions 4-8 and 13-18, optionally wherein the non-terminal nucleotide positions are positions 4, 6, 7 and 8, or are positions 5, 6, 7, 15, and 17 of the second sense strand, wherein the positions are independently counted starting at the 5′-termini of the first and second sense strands, respectively.

In certain embodiments, only one non-terminal nucleotide position of the first sense strand and only one non-terminal nucleotide position of the second sense strand independently have the following structure:

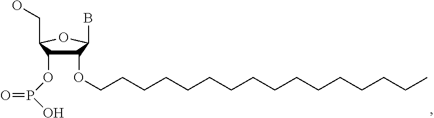

wherein B is a nucleotide base or a nucleotide base analog, optionally wherein B is adenine, guanine, cytosine, thymine or uracil, wherein the n-hexadecyl chain is the lipophilic moiety.

In some embodiments, the one non-terminal nucleotide position of the first sense strand is selected from the group consisting of positions 2-8 and 13-20 of the first sense strand, optionally selected from the group consisting of positions 4-8 and 13-18, optionally selected from the group consisting of positions 4-8, 15, and 17 of the first sense strand; and the one non-terminal nucleotide position of the second sense strand is selected from the group consisting of positions positions 2-8 and 13-20 of the first sense strand, optionally selected from the group consisting of positions 4-8 and 13-18, optionally selected from the group consisting of positions 4-8, 15, and 17 of the second sense strand, wherein the positions are independently counted starting at the 5′-termini of the first and second sense strands, respectively.

In certain embodiments, L is a bio-cleavable linker. All descriptions relating to bio-cleavable linkers in the above aspects or embodiments can be applicable herein for L. In certain embodiments, L may be

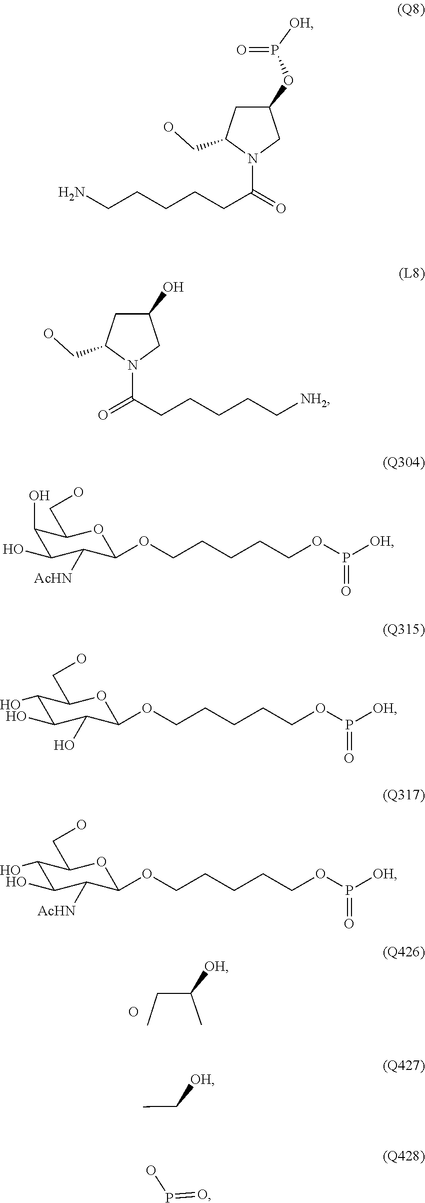

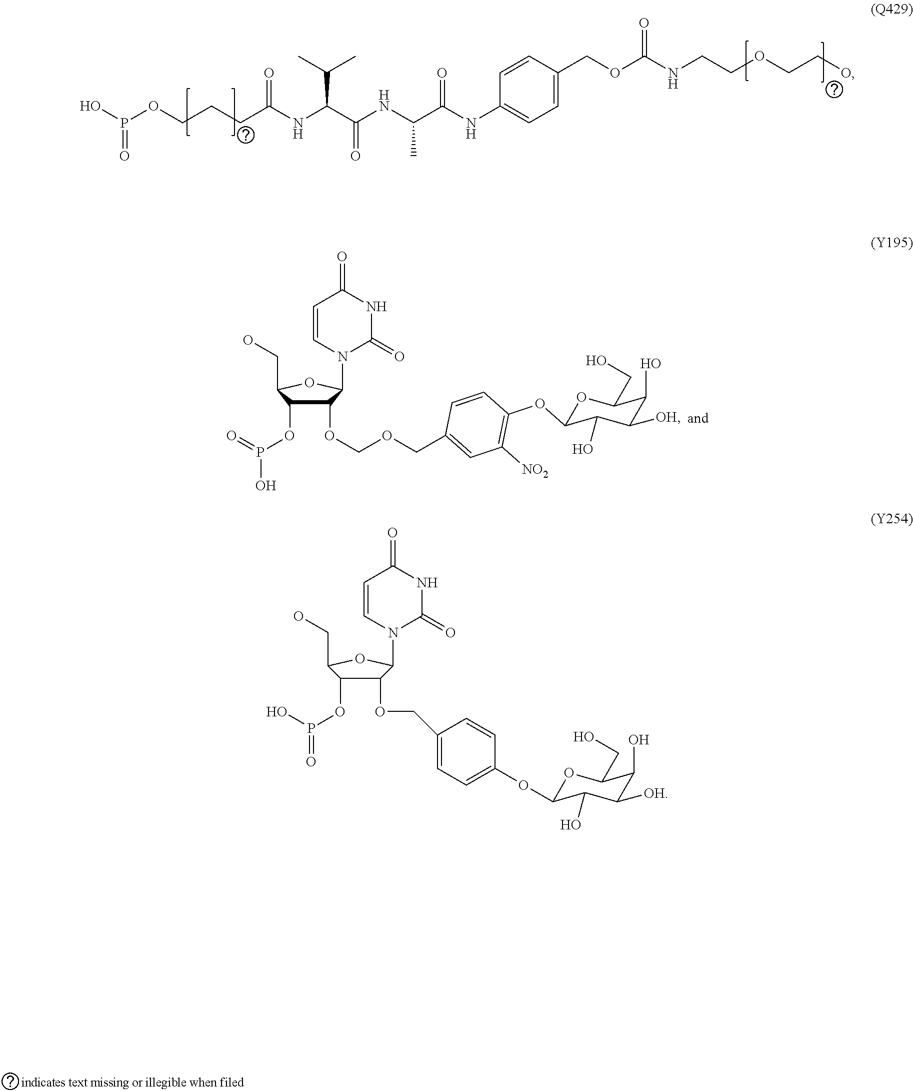

In certain embodiments, L is a redox cleavable linking group. Optionally, L is or includes a —S—S— or a —C(R)₂—S—S—, wherein R is H or C₁-C₆ alkyl and at least one R is C₁-C₆ alkyl, optionally CH₃ or CH₂CH₃.

In some embodiments, L is a phosphate-based cleavable linking group. Optionally, L is or includes —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O, —O—P(O)(H)—O—, —O—P(S)(H)—O—, —S—P(O)(H)—O—, —S—P(S)(H)—O—, —S—P(O)(H)—S—, or —O—P(S)(H)—S—, wherein R is optionally substituted linear or branched C₁-C₁₀ alkyl.

In certain embodiments, L is an acid cleavable linking group. Optionally, L is or includes hydrazones, esters, esters of amino acids, —C═NN— or —OC(O)—.

In some embodiments, L is an ester-based cleavable linking group. Optionally, L is or includes —C(O)O—.

In certain embodiments, L is a peptide-based cleavable linking group. Optionally, L is or includes a linking group that is cleaved by a cellular enzyme. Optionally, the cellular enzyme is a peptidase or a protease. Optionally, L is or includes —NHCHR^{A}C(O)NHCHR^{B}C(O)—, wherein R^{A} and R^{B} are the R groups of the two adjacent amino acids.

In some embodiments, a nucleic acid composition or pharmaceutical composition of the instant disclosure targets one or more target RNAs comprising two or more distinct target RNA sequences.

For the multi-targeted molecules of the instant disclosure, which possess at least one lipophilic moiety conjugated to each dsRNA, the surprisingly robust delivery and inhibitory efficacies observed for such molecules in the tissues of the CNS are noted, among other features, as distinguishing such molecules of the instant disclosure from the multi-targeted single entity conjugates described in PCT application no. PCT/US2016/042498.

Another aspect of the invention relates to a small circular interfering RNA (sciRNA) for modulating one or more target mRNAs in the central nervous system (CNS) of a subject, comprising a first strand having at least 40 nucleotides in length and at least two first strand nucleotide sequences connected together by a bis-linker, each nucleotide sequence having about 18 to about 28 nucleotides in length, and at least one second strand nucleotide sequence, having about 19 to about 23 nucleotides in length, annealed with at least one of the first strand nucleotide sequences. The first strand has a circular or substantially circular structure. Each of the first strand nucleotide sequences and the second strand nucleotide sequence(s) comprises at least one nucleic acid modification. The first strand nucleotide sequences or the second strand nucleotide sequence(s) comprise one or more ligands.

In some embodiments, each of the first strand nucleotide sequences and the second strand nucleotide sequence(s) is at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. Each of the first strand nucleotide sequences may have about 18 to about 28 nucleotides in length, e.g., about 19 to 25 nucleotides in length, about 19 to 23 nucleotides in length, or about 20 to 21 nucleotides in length. Each of the second strand nucleotide sequence(s) may have about 19 to about 25 nucleotides in length, about 19 to 23 nucleotides in length, or about 21 to 23 nucleotides in length.

In some embodiments, the first strand is an antisense strand, and the second strand nucleotide sequence is a sense strand nucleotide sequence. Thus, the antisense strand has a circular or substantially circular structure, and has at least two antisense strand nucleotide sequences connected together by a bis-linker. At least one of the antisense strand nucleotide sequences is annealed with a sense strand nucleotide sequence. In one embodiment, each of the antisense strand nucleotide sequence is annealed with a same or different sense strand nucleotide sequence.

In some embodiments, the first strand is a sense strand, and the second strand nucleotide sequence is an antisense strand nucleotide sequence. Thus, the sense strand has a circular or substantially circular structure, and has at least two sense strand nucleotide sequences connected together by a bis-linker. At least one of the sense strand nucleotide sequences is annealed with an antisense strand nucleotide sequence. In one embodiment, each of the sense strand nucleotide sequence is annealed with a same or different antisense strand nucleotide sequence.

In some embodiments, each of the circular or substantially circular sense strand nucleotide sequence is at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. In one embodiment, each of the sense strand nucleotide sequence is about 19 to 23 nucleotides in length, or about 20 to 21 nucleotides in length.

In some embodiments, each of the antisense strand nucleotide sequence(s) is at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. In one embodiment, each of the antisense strand nucleotide sequence is about 21 to 23 nucleotides in length, or 23 nucleotides in.

The antisense strand nucleotide sequence(s) is annealed with the circular or substantially circular sense strand nucleotide sequence(s). In some embodiments, one or more sense nucleotide sequences are annealed with the antisense strand nucleotide sequence(s). In some embodiments, each of the sense nucleotide sequences is annealed with an antisense strand nucleotide sequence. In some embodiments, at least one sense nucleotide sequence is not annealed with an antisense strand nucleotide sequence.

In some embodiments, the sense nucleotide sequence not annealed with an antisense strand nucleotide sequence can be an inhibitory single-stranded oligonucleotide, such as an antisense oligonucleotide (ASO), an antimiR (antagomir) oligonucleotide, or a single-stranded siRNA (ss-siRNA) oligonucleotide.

In some embodiments, a duplex region is formed between a sense strand nucleotide sequence and an antisense strand nucleotide sequence at least at the seed region of the antisense strand nucleotide sequence.

In certain embodiments, the circular or substantially circular sense strand comprises at least two symmetrical sense nucleotide sequences, each having about 19 to about 23 nucleotides in length. In one embodiment, the circular or substantially circular sense strand comprises at least two symmetrical sense nucleotide sequences, each having 20 to 21 nucleotides in length. By “symmetrical” is meant a same antisense nucleotide sequence can be annealed with either of the two sense nucleotide sequences. In one embodiment, the sense strand nucleotide sequences are annealed with at least two identical antisense nucleotide sequences, each having 23 nucleotides in length and targeting the same mRNA transcript nucleotide sequence. The sciRNA (bis-sciRNA) thus is capable of inhibiting the activity or expression of at least one target mRNA transcript in a tissue of the CNS of the subject.

In certain embodiments, the circular or substantially circular sense strand comprises at least two asymmetrical sense nucleotide sequences, each having about 19 to about 23 nucleotides in length. In one embodiment, the circular or substantially circular sense strand comprises at least two asymmetrical sense nucleotide sequences, each having 20 to 21 nucleotides in length. By “asymmetrical” is meant antisense nucleotide sequences that can be annealed with the at least two sense nucleotide sequences are different. In one embodiment, the sense strand nucleotide sequences are annealed with at least two different antisense nucleotide sequences, each having 23 nucleotides in length and targeting at least two different mRNA transcript nucleotide sequences. The sciRNA (bis-sciRNA) thus is capable of inhibiting the activity or expression of two or more distinct target mRNA transcripts in a tissue of the CNS of the subject. In one embodiment, the two or more distinct target mRNAs are located in the same nucleic acid.

In some embodiments, the bis-linker connecting the first strand (circular or substantially circular sense strand, or circular or substantially circular antisense strand) nucleotide sequences is an organic polymer linker. The organic polymer linker may be a bio-cleavable linker.

In some embodiments, the circular or substantially circular sense strand contains a nucleotide-based linker (tether). In some embodiments, the circular or substantially circular sense strand contains a non-nucleotide-based linker (tether). In one embodiment, the bis-linker connecting the sense nucleotide sequences is a nucleotide-based or a non-nucleotide-based linker (tether).

In some embodiments, the circular or substantially circular antisense strand contains a nucleotide-based linker (tether). In some embodiments, the circular or substantially circular antisense strand contains a non-nucleotide-based linker (tether). In one embodiment, the bis-linker connecting the antisense nucleotide sequences is a nucleotide-based or a non-nucleotide-based linker (tether).

In certain embodiments, the nucleotide-based or non-nucleotide-based linker (tether) is a stable linker (tether) that is stable in a biological fluid. For instance, the nucleotide-based or non-nucleotide based stable linker (tether) is stable in plasma or artificial cerebrospinal fluid.

In certain embodiments, the nucleotide-based or non-nucleotide-based linker (tether) is a cleavable linker (tether). For instance, the nucleotide-based or non-nucleotide based cleavable linker (tether) can be cleavable in liver homogenates, liver tritosomes, liver lysosomes, liver cytosol, brain homogenates, brain tritosomes, brain lysosomes, or brain cytosol.

In certain embodiments, the cleavable linker (tether) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., an ester group), or a peptidase cleavable linker (e.g., an ester group).

In certain embodiments, the cleavable linker comprises at least one modified internucleotide linkage selected from the group consisting of a phosphodiester, phosphotriester, hydrogen phosphonate, alkyl or aryl phosphonate, phosphoramidate, phosphorothioate, methylenemethylimino, thiodiester, thionocarbamate, N,N′-dimethylhydrazine, phosphoroselenate, borano phosphate, borano phosphate ester, amide, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, oxime, methyleneimino, methylenecarbonylamino, methylenehydrazo, methylenedimethylhydrazo, methyleneoxymethylimino, ether, thioacetamido, and combinations thereof.

In some embodiments, the bis-linker in the circular or substantially circular sense strand contains a nucleotide-based cleavable linker (tether) that is cleavable by DICER. In some embodiments, the circular or substantially circular sense strand comprises a substrate cleavable by DICER.

In some embodiments, the bis-linker in the circular or substantially circular antisense strand contains a nucleotide-based cleavable linker that is cleavable by DICER. In some embodiments, the circular or substantially circular antisense strand comprises a substrate cleavable by DICER.

In certain embodiments, the antisense strand forms circular or substantially circular structure, and contains a cleavable linker (nucleotide or non-nucleotide) capable of generating a metabolite of a 5′-monophosphate at an antisense nucleotide sequence of the antisense strand. The circular or substantially circular antisense strand can be cleaved to a linear structure that contains a metabolite of a 5′-monophosphate at an antisense nucleotide sequence of the antisense strand.

In some embodiments, the bis-linker connecting the first strand (circular or substantially circular sense strand, or circular or substantially circular antisense strand) nucleotide sequences comprises a bio-cleavable linker selected from the group consisting of DNA, RNA, disulfide, amide, functionalized monosaccharides or oligosaccharides of galactosamine, glucosamine, glucose, galactose, mannose, and combinations thereof.

In some embodiments, the bis-linker is an endosomal cleavable linker or a protease cleavable linker, for instance, a carbohydrate linker, wherein the linker is cleaved at least 1.25 times faster in the cell (or under in vitro conditions selected to mimic intracellular conditions) as compared to blood or serum (or under in vitro conditions selected to mimic extracellular conditions).

In certain embodiments, the bis-linker connecting the first strand (circular or substantially circular sense strand, or circular or substantially circular antisense strand) nucleotide sequences comprises a moiety selected from the group consisting of an aliphatic saturated or unsaturated alkyl chain; a phosphorous-containing linkage, including a phosphate, a phosphonate, a phosphoramidate, phosphodiester, phosphotriester, and phosphorothioate; a (poly)ethylene glycol chain, including diethylene glycol, triethylene glycol, tetra, penta, hexa, hepta, octa, nona, or deca ethylene glycol; glycerol or glycerol ester; an aminoalkyl ether; and combinations thereof.

In certain embodiments, the bis-linker connecting the first strand (circular or substantially circular sense strand, or circular or substantially circular antisense strand) nucleotide sequences comprises a moiety selected from the group consisting of

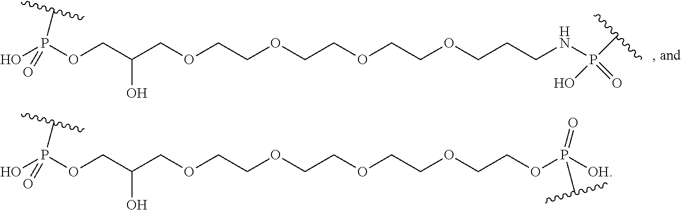

In certain embodiments, the bis-linker connecting the first strand (circular or substantially circular sense strand, or circular or substantially circular antisense strand) nucleotide sequences comprises a moiety selected from the following:
—(CH₂)₁₂— (C12 linker or Q50),
—(CH₂)₆—S—S—(CH₂)₆— (C6-S—S—C6 linker or Q51),

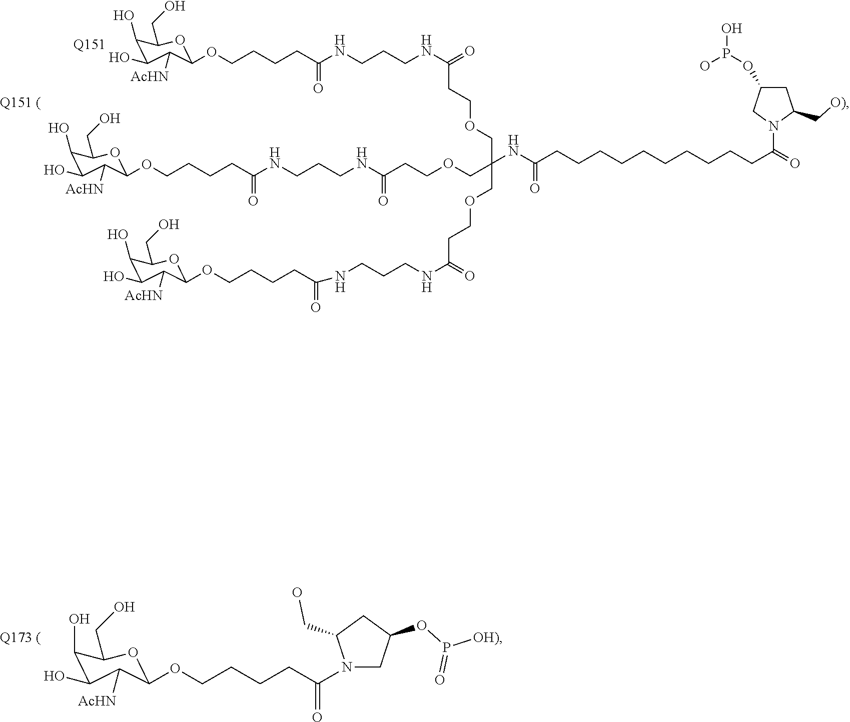

—CH₂CH₂O—(CH₂CH₂)ₙ—CH₂CH₂O—CH₂CH₂O—, wherein n is 0 or 1-20;
—(CH₂)₉—(CH₂)ₙ—CH₂—, wherein n is 0 or 1-20;
mono-, di-, tri-, tetra-, penta- or polyprolinol, optionally conjugated with a ligand;
mono-, di-, tri-, tetra-, penta- or polyhydroxyprolinol, optionally conjugated with a ligand.

In certain embodiments, the bis-linker connecting the first strand (circular or substantially circular sense strand, or circular or substantially circular antisense strand) nucleotide sequences comprises a nucleic acid linker of 1 to 15 nucleotides in length. For instance, the nucleic acid linker may be 2 to 5 nucleotides, 3 to 4 nucleotides, or 3 nucleotides in length.

In certain embodiments, the bis-linker connecting the first strand (circular or substantially circular sense strand, or circular or substantially circular antisense strand) nucleotide sequences comprises one or more sequences selected from the group consisting of UUU, 2′-O-methyl-UUU (uuu), 2′-fluoro-UUU (UfUfUf), and (dT)n, wherein n is 1-20 (e.g., dTdTdT).

In certain embodiments, the bis-linker connecting the first strand (circular or substantially circular sense strand, or circular or substantially circular antisense strand) nucleotide sequences comprises a nucleic acid linker comprising one or more nucleotides selected from the group consisting of 2′-O-methyl nucleotides, 2′-fluoro nucleotides, deoxyribonucleotides, and ribonucleotides. In one embodiment, all nucleic acid linker nucleotides are the same type of nucleotide. In one embodiment, wherein the nucleic acid linker entirely comprises 2′-O-methyl nucleotides, entirely comprises 2′-fluoro nucleotides, or entirely comprises deoxyribonucleotides.

In certain embodiments, the bis-linker connecting the first strand (circular or substantially circular sense strand, or circular or substantially circular antisense strand) nucleotide sequences comprises a polynucleotide comprising a modified ribonucleotide sequence, optionally a polynucleotide comprising one or more modifications selected from the group consisting of a 2′-O-methyl ribonucleotide modification, a 2′-fluoro-ribonucleotide modification, a 2′-5′-linked nucleotide with different 3′-modification (3′-ribo, 3′-O-methyl, 3′-deoxy, 3′-fluoro), a glycol nucleic acid (GNA) modification, a locked nucleic acid (LNA) modification, a hexanol nucleic acid (HNA) modification, an abasic ribose modification, an abasic deoxyribose modification, and an abasic hydroxyprolinol modification.

In certain embodiments, the bis-linker connecting the first strand (circular or substantially circular sense strand, or circular or substantially circular antisense strand) nucleotide sequences comprises one or more moieties selected from the group consisting of a phosphate diester linkage, a phosphate triester linkage (optionally comprising the linkage phosphorus atom in either Rp configuration or Sp configuration), a phosphorothioate diester linkage (optionally comprising the linkage phosphorus atom in either Rp configuration or Sp configuration), a phosphoramidate diester linkage (optionally comprising the linkage phosphorus atom in either Rp configuration or Sp configuration), and a disulfide linkage.

In certain embodiments, the circular or substantially circular sense strand has two nucleotide sequences, ss1 and ss2, and the 3′-end of the ss1 is connected to the 5′-end of ss2 by a bis-linker:

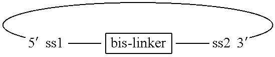

In other embodiments, the circular or substantially circular sense strand has two nucleotide sequences, ss1 and ss2, and the 3′-end of the ss1 is connected to the 3′-end of ss2 by a bis-linker:

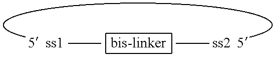

In other embodiments, the circular or substantially circular sense strand has two nucleotide sequences, ss1 and ss2, and the 5′-end of the ss1 is connected to the 5′-end of ss2 by a bis-linker:

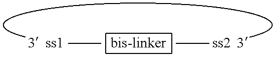

In one embodiment, ss1 is annealed with an antisense strand nucleotide sequence as1. The 3′-end of as1 may form a 3′-overhang of 1-2 nucleotides in length with respect to the 5′-end of ss1. The 5′-end of as1 may form a 5′-overhang of 1-2 nucleotides in length with respect to the 3′-end of ss1.

In one embodiment, ss2 is annealed with an antisense strand nucleotide sequence as2. The 3′-end of as2 may form a 3′-overhang of 1-2 nucleotides in length with respect to the 5′-end of ss2. The 5′-end of as2 may form a 5′-overhang of 1-2 nucleotides in length with respect to the 3′-end of ss2.

In certain embodiments, the bis-linker between ss1 and ss2 is represented by -(nt1)(nt2)(nt3)-, wherein each of nt1, nt2, and nt3 are independently a nucleotide or modified nucleotide. In some embodiments, nt1, nt2, and nt3 are each independently selected from the group consisting of A, T, U, G, C, and various modifications thereof. Each of A, T, U, G, C can be in a nucleotide form selected from the group consisting of 2′-O-methyl nucleotides, 2′-fluoro nucleotides, deoxyribonucleotides, and ribonucleotides. In one embodiment, nt1, nt2, and nt3 are one of the followings: UUU, 2′-O-methyl-UUU (uuu), 2′-fluoro-UUU (UfUfUf), or dTdTdT.

In one embodiment, ss1 is annealed with an antisense strand nucleotide sequence as1, and ss2 is annealed with an antisense strand nucleotide sequence as2:

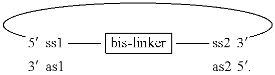

The antisense strand nucleotide sequences as1 and as2 may each comprise a 3′-overhang of 2 nucleotides in length. The antisense strand nucleotide sequences as1 and as2 may each comprise a 5′-overhang of 1-2 nucleotides in length.

In some embodiments, the bis-linker between ss1 and ss2 is a nucleic acid linker of 3 nucleotides in length; the antisense strand nucleotide sequences as1 and as2 each comprise a 3′-overhang of 2 nucleotides in length. In one embodiment, the two nucleotides of the 3′-overhang of as2 are complementary to the nucleotides of the bis-linker. In one embodiment, the two nucleotides of the 3′-overhang of as2 has one mismatch to the nucleotides of the bis-linker. In one embodiment, the two nucleotides of the 3′-overhang of as2 have two mismatches to the nucleotides of the bis-linker.

In another embodiment, ss1 is annealed with an antisense strand nucleotide sequence as1 and ss2 is annealed with an antisense strand nucleotide sequence as2:

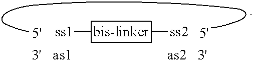

The antisense strand nucleotide sequences as1 and as2 may each comprise a 3′-overhang of 2 nucleotides in length. The antisense strand nucleotide sequences as1 and as2 may each comprise a 5′-overhang of 1-2 nucleotides in length.

In some embodiments, the bis-linker between ss1 and ss2 is a nucleic acid linker of 3 nucleotides in length; the antisense strand nucleotide sequences as1 and as2 each comprise a 5′-overhang of 1-2 nucleotides in length. In one embodiment, the one or two nucleotides of the 5′-overhang of as2 or as1 are complementary to the nucleotides of the bis-linker. In one embodiment, the one or two nucleotides of the 5′-overhang of as2 or as 1 has one mismatch to the nucleotides of the bis-linker. In one embodiment, the two nucleotides of the 5′-overhang of as2 or as1 have two mismatches to the nucleotides of the bis-linker.

In another embodiment, ss1 is annealed with an antisense strand nucleotide sequence as1, and ss2 is annealed with an antisense strand nucleotide sequence as2:

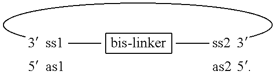

The antisense strand nucleotide sequences as1 and as2 may each comprise a 3′-overhang of 1-2 nucleotides in length. The antisense strand nucleotide sequences as1 and as2 may each comprise a 5′-overhang of 1-2 nucleotides in length.

In some embodiments, the bis-linker between ss1 and ss2 is a nucleic acid linker of 3 nucleotides in length; the antisense strand nucleotide sequences as1 and as2 each comprise a 3′-overhang of 1-2 nucleotides in length. In one embodiment, the one or two nucleotides of the 3′-overhang of as2 or as1 are complementary to the nucleotides of the bis-linker. In one embodiment, the one or two nucleotides of the 3′-overhang of as2 or as1 has one mismatch to the nucleotides of the bis-linker. In one embodiment, the two nucleotides of the 3′-overhang of as2 or as1 have two mismatches to the nucleotides of the bis-linker.

In some embodiments, the sciRNA comprises at least one chemical modification. The chemical modification may include an internucleoside linkage modification, a nucleobase modification, a sugar modification, or combinations thereof.

In certain embodiments, the chemical modification is selected from the group consisting of LNA, ENA, HNA, CeNA, 2′-O-methoxyalkyl (e.g., 2′-O-methoxymethyl, 2′-O-methoxyethyl, or 2′-O-2-methoxypropanyl), 2′-O-alkyl, 2′-O-allyl, 2′-C-allyl, 2′-fluoro, 2′-deoxy, 2′-O—N-methylacetamido (2′-O-NMA), 2′-O-dimethylaminoethoxyethyl (2′-O-DMAEOE), 2′-O-aminopropyl (2′-O-AP), 2′-ara-F, L-nucleoside modification (such as 2′-modified L-nucleoside, e.g., 2′-deoxy-L-nucleoside), BNA abasic sugar, abasic cyclic and open-chain alkyl, and combinations thereof.

In certain embodiments, the chemical modification is a 2′-modification selected from the group consisting of 2′-O-methyl, 2′-deoxy, 2′-fluoro, 2′-C₆-C₁₈ hydrocarbon chain, and combinations thereof.

In certain embodiments, the chemical modification is a 2′-modification selected from the group consisting of 2′-O-methyl, 2′-deoxy, 2′-fluoro, and combinations thereof.

In some embodiments, about 100%, 95%, 90%, 85%, 80%, 75%, 70%, 65%, 60%, 55%, 50%, 45%, 40%, 35% or 30% of all the nucleotides are modified. For example, when 50% of all the nucleotides are modified, 50% of all nucleotides present in the sciRNA contain a modification as described herein.

In some embodiments, all the nucleotides in the first strand (e.g., sense strand) nucleotide sequences are modified.

In some embodiments, all the nucleotides in the second strand (e.g., antisense strand) nucleotide sequence(s) are modified.

In one embodiment, at least 50% of the nucleotides of the sciRNA are independently modified with 2′-O-methyl, 2′-O-allyl, 2′-deoxy, or 2′-fluoro.

The sciRNA comprises one or more ligands.

In some embodiments, the circular or substantially circular sense strand may comprise one or more ligands. In one embodiment, each sense nucleotide sequence in the circular or substantially circular sense strand comprises at least one ligand, which may be particularly effective in modulating gene expression. Thus, at least two ligands (e.g., lipophilic ligands) are conjugated with the multi-targeted bis-sciRNA molecule.

In some embodiments, the antisense strand nucleotide sequences comprise one or more ligands. In one embodiment, each antisense nucleotide sequence comprises at least one ligand, which may be particularly effective in modulating gene expression. Thus, at least two ligands (e.g., lipophilic ligands) are conjugated with the multi-targeted bis-sciRNA molecule.

In certain embodiments, at least one of the ligands is conjugated to a strand that has a circular or substantially circular structure. In certain embodiments, at least one of the ligands is conjugated to a strand that does not have a circular or substantially circular structure. In one embodiment, at least one of the ligands is conjugated to a strand that has a circular or substantially circular structure, and at least one of the ligands is conjugated to a strand that does not have a circular or substantially circular structure.

In certain embodiments, at least one of the ligands is conjugated with a sense nucleotide sequence of the sense strand. At least one of the ligands may be conjugated at the 3′-end, 5′-end, or an internal position of the sense nucleotide sequence. In one embodiment, the conjugated sense strand has a circular or substantially circular structure. In one embodiment, the conjugated sense strand does not a circular or substantially circular structure.

In certain embodiments, at least one of the ligands is conjugated with an antisense nucleotide sequence of the antisense strand. At least one of the ligands may be conjugated at the 3′-end, 5′-end, or an internal position of the antisense nucleotide sequence. In one embodiment, the conjugated antisense strand has a circular or substantially circular structure. In one embodiment, the conjugated antisense strand does not a circular or substantially circular structure.

In some embodiments, the ligand may be conjugated to the sciRNA via a direct attachment to the ribosugar of the sciRNA. Alternatively, the ligand may be conjugated to the sciRNA via one or more linkers (tethers), and/or a carrier.

In some embodiments, the ligand may be conjugated to the sciRNA molecule via a monovalent or branched bivalent or trivalent linker.

In some embodiments, the ligand may be conjugated to the sciRNA via a carrier that replaces one or more nucleotide(s). The carrier can be a cyclic group or an acyclic group. In one embodiment, the cyclic group is selected from the group consisting of cyclohexyl, pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

In certain embodiments, at least one of the ligands is a lipophilic moiety.

In one embodiment, the lipophilic moiety is lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In some embodiments, the lipophilic moiety contains a saturated or unsaturated C₄-C₃₀ hydrocarbon chain (e.g., C₄-C₃₀ alkyl or alkenyl), and an optional functional group selected from the group consisting of hydroxyl, amine, carboxylic acid, sulfonate, phosphate, thiol, azide, and alkyne. In one embodiment, the lipophilic moiety contains a saturated or unsaturated C₆-C₁₈ hydrocarbon chain (e.g., a linear C₆-C₁₈ alkyl or alkenyl), e.g., a saturated or unsaturated C₁₆ or C₂₂ hydrocarbon chain (e.g., a linear C₁₆ or C₂₂ alkyl or alkenyl). For example, one or more non-terminal positions of the sense strand nucleotide sequences may have the following structure:

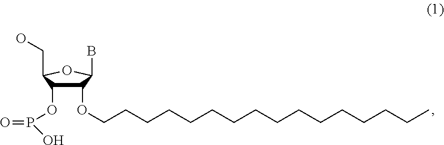

wherein B is a natural or modified nucleotide base (e.g., adenine, guanine, cytosine, thymine or uracil, or their modified derivatives), and the n-hexadecyl chain is the lipophilic moiety. The modification shown in formula (1) is referred to herein as “2′-C16”. Similar modifications replacing the n-hexadecyl chain with C₄-C₃₀ hydrocarbon chain is referred to as “2′-C₄-C₃₀ hydrocarbon chain” (or replacing with C₆-C₁₈ hydrocarbon chain is referred to as “2′-C₆-C₁₈ hydrocarbon chain”).

In a related embodiment, one or more non-terminal nucleotide positions of the sense strands of the of the sense or antisense strand nucleotide sequences have the 2′-C₄-C₃₀ hydrocarbon chain structure, 2′-C₆-C₁₈ hydrocarbon chain structure, or 2′-C16 structure of formula (1).

In one embodiment, one or more non-terminal nucleotide positions of all the sense strand nucleotide sequences have the 2′-C₄-C₃₀ hydrocarbon chain structure, 2′-C₆-C₁₈ hydrocarbon chain structure, or 2′-C16 structure of formula (1).

In one embodiment, one or more non-terminal nucleotide positions of all the antisense strand nucleotide sequences have the 2′-C₄-C₃₀ hydrocarbon chain structure, 2′-C₆-C₁₈ hydrocarbon chain structure, or 2′-C16 structure of formula (1).

In some embodiments, one or more of the circular or substantially circular sense strand nucleotide sequences comprise one or more lipophilic moieties conjugated independently to one or more of the non-terminal positions excluding positions 9-12 on a sense strand nucleotide sequence; for instance, positions 4-8 and 13-18 on a sense strand nucleotide sequence; positions 5, 6, 7, 15, and 17 on a sense strand nucleotide sequence; or positions 4, 6, 7, and 8 on a sense strand nucleotide sequence, counting from the 5′-end of the sense strand nucleotide sequence as position 1.

In some embodiments, one or more of the circular or substantially circular sense strand nucleotide sequences comprises one or more lipophilic moieties conjugated independently to position 6 of the nucleotide sequence, counting from the 5′-end of the nucleotide sequence. In one embodiment, each sense strand nucleotide sequence comprises a lipophilic moiety conjugated to position 6 of the nucleotide sequence; optionally the lipophilic moiety comprises a saturated or unsaturated C₆-C₁₈ hydrocarbon chain; optionally the lipophilic moiety comprises a saturated or unsaturated C₁₆ hydrocarbon chain.

In some embodiments, one or more of the antisense strand nucleotide sequences comprise one or more lipophilic moieties conjugated independently to one or more of non-terminal positions on an antisense strand nucleotide sequence; for instance, positions 6-10 and 15-18 on an antisense strand nucleotide sequence; and positions 15 and 17 on an antisense strand nucleotide sequence, counting from the 5′-end of the antisense strand nucleotide sequence as position 1.

In certain embodiments, at least one of the ligands is a carbohydrate-based ligand. The carbohydrate-based ligand may be D-galactose, multivalent galactose, N-acetyl-D-galactosamine (GalNAc), multivalent GalNAc, D-mannose, multivalent mannose, multivalent lactose, N-acetyl-glucosamine, glucose, multivalent glucose, multivalent fucose, glycosylated polyaminoacids, or lectins.

In certain embodiments, the carbohydrate-based ligand is an ASGPR ligand. For example, the ASGPR ligand is one or more GalNAc derivatives attached through a bivalent or trivalent branched linker, such as:

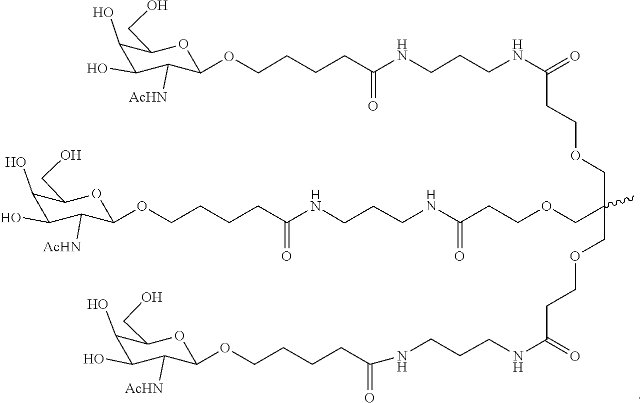 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the antisense strand nucleotide sequence(s) comprises a phosphate or phosphate mimic at the 5′-end of an antisense strand nucleotide sequence. In one embodiment, at least one phosphate mimic is at the 5′ end of each antisense nucleotide sequence.

The phosphate mimic can be 5′-end phosphorothioate (5′-PS), 5′-end phosphorodithioate (5′-PS2), 5′ end vinylphosphonate (5′-VP), 5′-end methylphosphonate (MePhos), or 5′-deoxy-5′-C-malonyl. In one embodiment, the phosphate mimic is a 5′-vinylphosphonate (VP). The 5′-VP can be either 5′-E-VP isomer (i.e., trans-vinylphosphate), 5′-Z-VP isomer (i.e., cis-vinylphosphate), or mixtures thereof.

In one embodiment, the phosphate mimic is a 5′-vinyl phosphonate (VP).

In some embodiments, the sciRNA further comprises at least one terminal, chiral phosphorus atom.

A site specific, chiral modification to the internucleotide linkage may occur at the 5′ end, 3′ end, or both the 5′ end and 3′ end of a sense or antisense nucleotide sequence. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a sense or antisense nucleotide sequence. A chiral modification may occur on the sense strand nucleotide sequence, antisense strand nucleotide sequence, or both the sense strand and antisense strand nucleotide sequences. Each of the chiral pure phosphorus atoms may be in either Rp configuration or Sp configuration, and combination thereof. More details regarding chiral modifications and chirally-modified dsRNA agents can be found in WO 2019/126651A1, which is incorporated herein by reference in its entirety.

In some embodiments, the sciRNA comprises at least two blocks of two consecutive phosphorothioate or methylphosphonate internucleotide linkage modifications.

In some embodiments, the sciRNA has at least two phosphorothioate internucleotide linkages at the first five nucleotides on an antisense strand nucleotide sequence (counting from the 5′ end).

In some embodiments, an antisense nucleotide sequence comprises two blocks of one, two, or three phosphorothioate internucleotide linkages separated by 1, 2, 3, 4, 5, 6, 7, 8, 9, 10, 11, 12, 13, 14, 15, 16, 17, or 18 phosphate internucleotide linkages.

In one embodiment, an antisense strand nucleotide sequence comprises at least two consecutive phosphorothioate internucleotide linkage modifications within positions 18-23 of an antisense nucleotide sequence, counting from the 5′-end of the antisense nucleotide sequence. A sense strand nucleotide sequence comprises at least two consecutive phosphorothioate internucleotide linkage modifications within position 1-5 of the sense nucleotide sequence, counting from the 5′-end of the sense nucleotide sequence.

In some embodiments, each of the nucleotide sequences of the sciRNA comprises at least two blocks of two consecutive phosphorothioate internucleotide linkage modifications. In one embodiment, each of the nucleotide sequences of the sciRNA comprises: at least two consecutive phosphorothioate internucleotide linkage modifications within positions 18-23 of the nucleotide sequence, and at least two consecutive phosphorothioate internucleotide linkage modifications within position 1-5 of the nucleotide sequence, counting from the 5′-end of the nucleotide sequence.

In some embodiments, the sciRNA comprises the following features: the circular or substantially circular sense strand has two nucleotide sequences, ss1 and ss2, and the 3′-end of the ss1 is connected to the 5′-end of ss2 by a bis-linker, wherein ss1 is annealed with an antisense strand nucleotide sequence as1, and ss2 is annealed with an antisense strand nucleotide sequence as2:

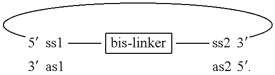

as1 and as2 each comprise a 3′-overhang of 2 nucleotides in length, and/or
as1 and as2 each comprise a 5′-overhang of 1 nucleotide in length;
all the nucleotides in ss1, ss2, as1, and as2 are modified;
the sciRNA comprises at least two blocks of two consecutive phosphorothioate or methylphosphonate internucleotide linkage modifications;
one or both of as1 and as2 comprises a phosphate mimic at the 5′-end, selected from the group consisting of 5′-phosphorothioate (5′-PS), 5′-phosphorodithioate (5′-PS2), 5′-vinylphosphonate (5′-VP), 5′-methylphosphonate (5′-MePhos), and 5′-deoxy-5′-C-malonyl; and
one or both of ss1 and ss2 comprises one or more ligands.

In one embodiment, the sciRNA comprises the following features:
the bis-linker between ss1 and ss2 is a nucleic acid linker of 3 nucleotides in length;
all the nucleotides in ss1, ss2, as1, and as2 are modified with a 2′-O-methyl or 2′-fluoro modification;
each of as1 and as2 comprises at least two consecutive phosphorothioate internucleotide linkage modifications within positions 18-23 of the nucleotide sequence, counting from the 5′-end of the nucleotide sequence; and each of ss1 and ss2 comprises at least two consecutive phosphorothioate internucleotide linkage modifications within position 1-5 of the nucleotide sequence, counting from the 5′-end of the nucleotide sequence; and
one or both of ss1 and ss2 comprises:
   one or more lipophilic moieties conjugated to position 6 of the nucleotide sequence, counting from the 5′-end of the nucleotide sequence; or
   at least one carbohydrate-based ligand conjugated at the 3′-end of the nucleotide sequence.

In some embodiments, the sense strand forms circular or substantially circular structure via a cycling linking moiety that connects one end of the sense strand to the other end of the sense strand.

In some embodiments, the antisense strand forms circular or substantially circular structure via a cycling linking moiety that connects one end of the antisense strand to the other end of the antisense strand.

In certain embodiments, the cycling linking moiety may contain one or more linkages selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In certain embodiments, the cycling linking moiety may contain one or more cyclic groups selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl.

In certain embodiments, the cycling linking moiety also serves as the carrier that carries a ligand and connect the ligand to the sciRNA.

Another aspect of the invention relates to a pharmaceutical composition comprising a sciRNA for modulating one or more target mRNAs in the central nervous system (CNS) of a subject and a pharmaceutically acceptable excipient. The sciRNA comprises a first strand having at least 40 nucleotides in length and at least two first strand nucleotide sequences connected together by a bis-linker, each nucleotide sequence having about 18 to about 28 nucleotides in length, and at least one second strand nucleotide sequence, having about 19 to about 23 nucleotides in length, annealed with at least one of the first strand nucleotide sequences. The first strand has a circular or substantially circular structure. Each of the first strand nucleotide sequences and the second strand nucleotide sequence(s) comprises at least one nucleic acid modification. The first strand nucleotide sequences or the second strand nucleotide sequence(s) comprise one or more ligands.

All the above embodiments relating to the first strand, first strand nucleotide sequences, second strand nucleotide sequence(s), sense strand, sense strand nucleotide sequences, antisense strand, antisense strand nucleotide sequences, the chemical modifications on the sense and antisense strand nucleotide sequences, the bis-linker, the nucleotide-based and non-nucleotide-based linkers, the ligand and ligand conjugation, and the cycling linking moiety disclosed in the first aspect of the invention relating to the sciRNA for modulating one or more target mRNAs in the central nervous system (CNS) of a subject are suitable in this aspect of the invention relating to a pharmaceutical composition.

Another aspect of the invention relates to a method for inhibiting the expression of one or more target mRNAs in the central nervous system (CNS) of in a subject, comprising contacting the CNS cell of the subject with a sciRNA for modulating one or more target mRNAs in the central nervous system (CNS) of a subject, in an amount sufficient to inhibit the activity or expression of the one or more target mRNAs in the CNS cell of the subject. The sciRNA comprises a first strand having at least 40 nucleotides in length and at least two first strand nucleotide sequences connected together by a bis-linker, each nucleotide sequence having about 18 to about 28 nucleotides in length, and at least one second strand nucleotide sequence, having about 19 to about 23 nucleotides in length, annealed with at least one of the first strand nucleotide sequences. The first strand has a circular or substantially circular structure. Each of the first strand nucleotide sequences and the second strand nucleotide sequence(s) comprises at least one nucleic acid modification. The first strand nucleotide sequences or the second strand nucleotide sequence(s) comprise one or more ligands.

All the above embodiments relating to the first strand, first strand nucleotide sequences, second strand nucleotide sequence(s), sense strand, sense strand nucleotide sequences, antisense strand, antisense strand nucleotide sequences, the chemical modifications on the sense and antisense strand nucleotide sequences, the bis-linker, the nucleotide-based and non-nucleotide-based linkers, the ligand and ligand conjugation, and the cycling linking moiety disclosed in the first aspect of the invention relating to the sciRNA for modulating one or more target mRNAs in the central nervous system (CNS) of a subject are suitable in this aspect of the invention relating to a method for inhibiting the expression of one or more target mRNAs in the central nervous system (CNS) of a subject.

In some embodiments, the cell is within a subject. In one embodiment, the subject is a human. In one embodiment, the subject is a non-human mammal, e.g., a rhesus monkey, a cynomolgous monkey, a mouse, or a rat.

Another aspect of the invention relates to a method of treating or preventing a CNS disease or disorder in a subject, comprising: administering to the subject a therapeutically effective amount of a sciRNA for modulating one or more target mRNAs in the central nervous system (CNS) of a subject, thereby treating or preventing the CNS disease or disorder in the subject. The sciRNA comprises a first strand having at least 40 nucleotides in length and at least two first strand nucleotide sequences connected together by a bis-linker, each nucleotide sequence having about 18 to about 28 nucleotides in length, and at least one second strand nucleotide sequence, having about 19 to about 23 nucleotides in length, annealed with at least one of the first strand nucleotide sequences. The first strand has a circular or substantially circular structure. Each of the first strand nucleotide sequences and the second strand nucleotide sequence(s) comprises at least one nucleic acid modification. The first strand nucleotide sequences or the second strand nucleotide sequence(s) comprise one or more ligands.

All the above embodiments relating to the first strand, first strand nucleotide sequences, second strand nucleotide sequence(s), sense strand, sense strand nucleotide sequences, antisense strand, antisense strand nucleotide sequences, the chemical modifications on the sense and antisense strand nucleotide sequences, the bis-linker, the nucleotide-based and non-nucleotide-based linkers, the ligand and ligand conjugation, and the cycling linking moiety disclosed in the first aspect of the invention relating to the sciRNA for modulating one or more target mRNAs in the central nervous system (CNS) of a subject are suitable in this aspect of the invention relating to a method for treating or preventing a CNS disease or disorder in a subject.

In all the above aspects of the invention, the sciRNA is capable of inhibiting the activity or expression of the one or more target mRNAs in a tissue of the CNS of the subject by at least 15% each relative to an appropriate control (e.g., as compared to an untreated or placebo-treated subject, or as compared to a reference value, including, e.g., target mRNA or protein levels in the treated subject measured before the treatment with the sciRNA occurred), optionally by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% each relative to an appropriate control. In one embodiment, the appropriate control is an untreated subject. In one embodiment, the appropriate control is a reference value, e.g., a value obtained for the subject prior to administration of the sciRNA to the subject.

In related embodiments, the sciRNA is capable of inhibiting expression of a target mRNA throughout the CNS of a subject, or within a location within the CNS of a subject. In certain embodiments, the sciRNA is capable of inhibiting expression of a target mRNA in one or more of the following CNS locations of a subject: right hemisphere, left hemisphere, cerebellum, striatum, brainstem, and spinal cord. CNS cell types targeted include, but are not limited to, neurons, oligodendrocytes, microglia, and astrocytes, among others.

In certain embodiments, the sciRNA may be formulated for intrathecal or intracerebroventricular (ICV) administration. In certain embodiments, in the methods described herein, the step of contacting or administering involves administering an intrathecal or intracerebroventricular (ICV) injectate to the subject.

In certain embodiments, the two or more distinct target mRNAs are transcripts of genes associated with a CNS disease or disorder.

Exemplary CNS diseases or disorders include a neurodegenerative disorder (e.g., Parkinson's Disease (PD), Alzheimer's disease, early onset familial Alzheimer's disease (EOFAD), cerebral amyloid angiopathy (CAA), Spinal Muscular Atrophy (SMA), Angelman Syndrome, ataxias/neurodegenerative disorders of the nervous system (e.g., Friedreich's Ataxia), Huntington's disease (Huntington chorea), multiple sclerosis, amyotrophic lateral sclerosis (ALS)), depression, Down's syndrome, psychosis, schizophrenia, Creutzfeldt-Jakob disease, multiple system atrophy, Lewy body dementia (LBD), pure autonomic failure (PAF), Pick's disease, progressive supranuclear palsy, dementia pugilistica, parkinsonism linked to chromosome 17, Lytico-Bodig disease, tangle predominant dementia, Argyrophilic grain disease, ganglioglioma, gangliocytoma, meningioangiomatosis, subacute sclerosing panencephalitis, lead encephalopathy, tuberous sclerosis, Hallervorden-Spatz disease, lipofuscinosis, corticobasal degeneration, frontotemporal dementia, frontotemporal lobar degeneration, a vascular disorder (e.g., stroke, transient ischemic attack (TIA), subarachnoid hemorrhage, subdural hemorrhage and hematoma, and extradural hemorrhage), an infection (e.g., meningitis, encephalitis, polio, epidural abscess), a structural disorders (e.g., brain or spinal cord injury, Bell's palsy, cervical spondylosis, carpal tunnel syndrome, brain or spinal cord tumors, peripheral neuropathy, Guillain-Barre syndrome), and a functional disorder (e.g., headache, epilepsy, dizziness, neuralgia).

### BRIEF DESCRIPTION OF THE DRAWINGS

FIGS. 1A and 1B show the structures and respective CNS-directed inhibitory efficacies (when administered by ICV injection to mice as mixed siRNAs) for the two siRNA molecules that were joined together to form the bis-siRNA complexes exemplified herein. FIG. 1A shows the sequence, structure and modification patterning of the SOD-targeting siRNA (top, including sense strand sequence 5′-CAUUUUAAUCCUCACUCUAAA-3′ (SEQ ID NO: 1) and antisense strand sequence 5′-UUUAGAGUGAGGAUUAAAAUGAG-3′ (SEQ ID NO: 2)) and the CTNNB1-targeting siRNA (bottom, including sense strand sequence 5′-UACUGUUGGAUUGAUUCGAAA-3′ (SEQ ID NO: 3) and antisense strand sequence 5′-UUUCGAAUCAAUCCAACAGUAGC-3′ (SEQ ID NO: 4). FIG. 1B shows the respective SOD1 and CTNNB1 inhibitory efficacies observed when the two siRNA molecules were administered as a 100 μg mixture by ICV injection to mice, with levels of inhibition measured at day 21 in the right hemisphere, left hemisphere, cerebellum and brainstem within the brain, as well as in the liver. Mouse number 8 was identified as an unsuccessful injection.

FIGS. 2A-2D show exemplary bis-siRNA complexes made and tested herein for tandem inhibition of mCTNNB1 and mSOD1. FIG. 2A summarizes the duplex identifier, sense strand identifier, linker, and configuration patterns for the exemplified bis siRNA complexes, as well as for the control mixed duplexes. All bis siRNA complexes included two sets of 21-mer sense strands and 23-mer antisense strands, wherein the sense strands of both siRNAs were made continuous via inclusion of a three-nucleotide single-stranded linker, while the respective antisense strands of siRNA duplexes were independent strands that were non-continuous. The configuration pattern of the duplexes as shown includes the order of the RNAi target duplexes and the number of C16 modifications for each bis complex. Numbers of mice in tested cohorts, day of target inhibitory assessment, does employed and locations of readouts obtained are also shown. FIG. 2B shows the complete sense strand of various different bis siRNA complexes, as indicated, including the linker for each bis complex (from top, SEQ ID NOs: 5-12, as shown in Table 3 herein, noting modified forms of these sequences listed as SEQ ID NOs: 17-24 in Table 2 herein). Modifications present on each displayed oligonucleotide sequence are indicated, with reference made to the key at right of each strand, including “DNA” for a DNA nucleotide, “2′OMe” for a 2′-O-methyl-modified nucleotide, “F” for a 2′-Fluoro-modified nucleotide, “PS” for a 3′ phosphorothioate modified-nucleotide, and “2-C16” for a C₁₆-modified nucleotide. FIG. 2C summarizes the configuration pattern and linkers used for each bis siRNA duplex of the instant disclosure. FIG. 2D shows the independent antisense strands (SEQ ID NO: 4 at top and SEQ ID NO: 2 at bottom, as summarized in Table 3 herein) respectively complementing the CTNNB1 and SOD1 sense strands that were linked, with the complex of the two respective antisense strand sequences shown hybridized to a fused sense strand sequence of FIG. 2B to form the form the various bis siRNA complexes tested herein.

FIGS. 3A-3C show the structure of a CTNNB1(C16)-SOD1(C16) bis siRNA multi-targeted molecule having respective siRNA effector molecule sense strands joined by a three nucleotide DNA linker (dTdTdT), as well as respective mSOD1 and mCTNNB1 levels of inhibition observed in mice injected with this construct. FIG. 3A shows the sequence and modifications of the CTNNB1(C16)-SOD1(C16) bis siRNA complex having a DNA (dTdTdT) linker, including “DNA” for DNA nucleotide, “2′OMe” for 2′-O-methyl modified nucleotide, “F” for 2′-Fluoro modified nucleotide, “PS” for 3′ phosphorothioate modified nucleotide, and “2-C16” for a C16-modified nucleotide. Respective sequences shown are fused sense strand sequence SEQ ID NO: 17, CTNNB1 antisense strand sequence SEQ ID NO: 16 and SOD1 antisense strand sequence SEQ ID NO: 14. FIG. 3B shows, for each individual mouse dosed, the percentage of SOD1 (top) and CTNNB1 (bottom) remaining at day 21 after a 100 μg ICV injection of the CTNNB1(C16)-SOD1(C16) bis siRNA molecule, in each of the indicated tissues (right hemisphere of the brain, left hemisphere of the brain, cerebellum, brainstem, and liver), for a cohort of five animals (noting that three animals—numbers 9, 10 and 11—reflected unsuccessful injections, for which data were removed from certain analyses). FIG. 3C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across either all five injected animals (top) or only for the two animals with successful injections (bottom).

FIGS. 4A-4C show the structure of a CTNNB1(C16)-SOD1(C16) bis siRNA multi-targeted molecule having respective siRNA effector molecule sense strands joined by a three nucleotide 2′O-methyl linker (uuu), as well as respective mSOD1 and mCTNNB1 levels of inhibition observed in mice injected with this construct. FIG. 4A shows the sequence and modifications of the CTNNB1(C16)-SOD1(C16) bis siRNA complex having a 2′O-methyl linker (uuu), including “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are fused sense strand sequence SEQ ID NO: 18, CTNNB1 antisense strand sequence SEQ ID NO: 16 and SOD1 antisense strand sequence SEQ ID NO: 14. FIG. 4B shows, for each individual mouse dosed, the percentage of SOD1 (top) and CTNNB1 (bottom) remaining at day 21 after a 100 μg ICV injection of the CTNNB1(C16)-SOD1(C16) bis siRNA molecule, in each of the indicated tissues (right hemisphere of the brain, left hemisphere of the brain, cerebellum, brainstem, and liver), for a cohort of four animals. FIG. 4C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across all four injected animals.

FIGS. 5A-5C show the structure of a CTNNB1(C16)-SOD1(C16) bis siRNA multi-targeted molecule having respective siRNA effector molecule sense strands joined by a three nucleotide RNA linker (UUU), as well as respective mSOD1 and mCTNNB1 levels of inhibition observed in mice injected with this construct. FIG. 5A shows the sequence and modifications of the CTNNB1(C16)-SOD1(C16) bis siRNA complex having a RNA linker (UUU), including “RNA” for unmodified ribonucleotide, “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are fused sense strand sequence SEQ ID NO: 19, CTNNB1 antisense strand sequence SEQ ID NO: 16 and SOD1 antisense strand sequence SEQ ID NO: 14. FIG. 5B shows, for each individual mouse dosed, the percentage of SOD1 (top) and CTNNB1 (bottom) remaining at day 21 after a 100 μg ICV injection of the CTNNB1(C16)-SOD1(C16) bis siRNA molecule, in each of the indicated tissues (right hemisphere of the brain, left hemisphere of the brain, cerebellum, brainstem, and liver), for a cohort of four animals. FIG. 5C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across all four injected animals.

FIGS. 6A-6C show the structure of a CTNNB1(C16)-SOD1(C16) bis siRNA multi-targeted molecule having respective siRNA effector molecule sense strands joined by a three nucleotide 2′-Fluoro linker (UfUfUf), as well as respective mSOD1 and mCTNNB1 levels of inhibition observed in mice injected with this construct. FIG. 6A shows the sequence and modifications of the CTNNB1(C16)-SOD1(C16) bis siRNA complex having a 2′-Fluoro linker (UfUfUf), including “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are fused sense strand sequence SEQ ID NO: 20, CTNNB1 antisense strand sequence SEQ ID NO: 16 and SOD1 antisense strand sequence SEQ ID NO: 14. FIG. 6B shows, for each individual mouse dosed, the percentage of SOD1 (top) and CTNNB1 (bottom) remaining at day 21 after a 100 μg ICV injection of the CTNNB1(C16)-SOD1(C16) bis siRNA molecule, in each of the indicated tissues (right hemisphere of the brain, left hemisphere of the brain, cerebellum, brainstem, and liver), for a cohort of four animals. FIG. 6C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across all four injected animals.

FIGS. 7A-7C show the structure of a SOD1(C16)-CTNNB1(C16) bis siRNA multi-targeted molecule having respective siRNA effector molecule sense strands joined by a three nucleotide DNA linker (dTdTdT), as well as respective mSOD1 and mCTNNB1 levels of inhibition observed in mice injected with this construct. FIG. 7A shows the sequence and modifications of the SOD1(C16)-CTNNB1(C16) bis siRNA complex having a DNA (dTdTdT) linker, including “DNA” for DNA nucleotide, “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are fused sense strand sequence SEQ ID NO: 22, CTNNB1 antisense strand sequence SEQ ID NO: 16 and SOD1 antisense strand sequence SEQ ID NO: 14. FIG. 7B shows, for each individual mouse dosed, the percentage of SOD1 (top) and CTNNB1 (bottom) remaining at day 21 after a 100 μg ICV injection of the SOD1(C16)-CTNNB1(C16) bis siRNA molecule, in each of the indicated tissues (right hemisphere of the brain, left hemisphere of the brain, cerebellum, brainstem, and liver), for a cohort of four animals (noting that one animal—number 30—reflected an unsuccessful injection, for which data were removed from certain subsequent analyses). FIG. 7C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across either all four injected animals (top) or only for the three animals with successful injections (bottom).

FIGS. 8A-8C show the structure of a SOD1(C16)-CTNNB1(C16) bis siRNA multi-targeted molecule having respective siRNA effector molecule sense strands joined by a three nucleotide 2′O-methyl linker (uuu), as well as respective mSOD1 and mCTNNB1 levels of inhibition observed in mice injected with this construct. FIG. 8A shows the sequence and modifications of the SOD1(C16)-CTNNB1(C16) bis siRNA complex having a 2′O-methyl linker (uuu), including “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are fused sense strand sequence SEQ ID NO: 23, CTNNB1 antisense strand sequence SEQ ID NO: 16 and SOD1 antisense strand sequence SEQ ID NO: 14. FIG. 8B shows, for each individual mouse dosed, the percentage of SOD1 (top) and CTNNB1 (bottom) remaining at day 21 after a 100 μg ICV injection of the SOD1(C16)-CTNNB1(C16) bis siRNA molecule, in each of the indicated tissues (right hemisphere of the brain, left hemisphere of the brain, cerebellum, brainstem, and liver), for a cohort of four animals. FIG. 8C shows the aggregated respective percentages of SOD1 and CTNNB1 observed as remaining at day 21 after a 100 μg ICV injection, as measured in the right hemisphere of the brain, left hemisphere of the brain, cerebellum, brainstem, and liver, with results aggregated and analyzed across all four injected animals.

FIGS. 9A and 9B show the structures of respective CTNNB1-SOD1(C16) and CTNNB1(C16)-SOD1 bis siRNA multi-targeted molecules having respective siRNA effector molecule sense strands joined by a three nucleotide DNA linker (dTdTdT) and with only one effector molecule within each multi-targeted molecule possessing a C₁₆-modified nucleotide (located within individual effector molecules as indicated). mSOD1 and mCTNNB1 levels of inhibition observed in mice injected with these constructs are also shown. FIG. 9A shows the sequence and modifications of the respective CTNNB1-SOD1(C16) and CTNNB1(C16)-SOD1 bis siRNA complexes having a DNA (dTdTdT) linker, including “DNA” for DNA nucleotide, “2′OMe” for 2′-O-methyl modified nucleotide, “F” for 2′-Fluoro modified nucleotide, “PS” for 3′ phosphorothioate modified nucleotide, and “2-C16” for a C₁₆-modified nucleotide. Respective sequences shown are, for the CTNNB1-SOD1(C16) bis siRNA complex at top, the fused sense strand sequence of SEQ ID NO: 21, CTNNB1 antisense strand sequence SEQ ID NO: 16 and SOD1 antisense strand sequence SEQ ID NO: 14, and for the CTNNB1(C16)-SOD1 bis siRNA complex at bottom, the fused sense strand sequence of SEQ ID NO: 24, CTNNB1 antisense strand sequence SEQ ID NO: 16 and SOD1 antisense strand sequence SEQ ID NO: 14. FIG. 9B shows observed levels of SOD1 and CTNNB1 remaining at day 21 after a 100 μg ICV injection of the CTNNB1-SOD1(C16) bis siRNA molecule (left) and of the CTNNB1(C16)-SOD1 bis siRNA molecule (right), in each of the indicated tissues (right hemisphere of the brain, left hemisphere of the brain, cerebellum, brainstem, and liver), obtained from respective cohorts of four animals each.

FIGS. 10A-10E compare the target gene inhibitory efficiencies observed for the various bis siRNA complexes disclosed herein targeting SOD1 and CTNNB1, including the respective duplexes of FIGS. 3A-3C, 4A-4C, 5A-5C, 6A-6C, 7A-7C, 8A-8C, 9A and 9B above, relative to a mixed siRNA delivery format, across all assayed CNS tissues (right hemisphere, left hemisphere, cerebellum and brainstem). FIG. 10A shows the SOD1 (left) and CTNNB1 (right) levels observed as remaining following 21 days of treatment with each of the indicated bis siRNA complexes (those shown in FIGS. 3A, 4A, 5A and 6A above, respectively), also compared to a mixed siRNA treatment format, measured in the right hemisphere of the brain, left hemisphere of the brain, cerebellum, and brainstem. Notably, the best inhibitory activity was observed for the bis siRNA complex having a DNA (dTdTdT) linker. FIG. 10B shows the SOD1 (left) and CTNNB1 (right) levels observed as remaining following 21 days of treatment with each of the indicated bis siRNA complexes (those shown in FIGS. 3A, 4A, 7A and 8A above, respectively), also compared to a mixed siRNA treatment format, measured in the right hemisphere of the brain, left hemisphere of the brain, cerebellum, and brainstem. Notably, flipping the position of CTNNB1 and SOD1 effector molecules within the multi-targeted molecule resulted in reduced activity for the bis siRNA complexes having the SOD1(C16)-CTNNB1(C16) configuration. FIG. 10C demonstrates the levels of SOD1 (left) and CTNNB1 (right) inhibition observed for a mixture of siRNAs, a robustly effective CTNNB1(C16)-SOD1(C16) bis siRNA duplex having a DNA linker (dTdTdT), as well as the surprising absence of inhibition observed for two respective bis siRNA duplexes possessing a C₁₆ modification on only one of the two effector molecules, CTNNB1-SOD1(C16) bis siRNA (having a C₁₆ modification on only the SOD1-targeting siRNA effector molecule) and CTNNB1(C16)-SOD1 bis siRNA (having a C₁₆ modification on only the CTNNB1-targeting siRNA effector molecule), as assessed in the right hemisphere of the brain, left hemisphere of the brain, cerebellum, and brainstem. FIG. 10D shows a comparison of the effects on SOD1 (left) and CTNNB1 (right) levels observed across all tested bis siRNA multi-targeted molecules, as well as a mixed siRNA control, segregated by target gene and as measured in all tested brain tissues (right hemisphere of the brain, left hemisphere of the brain, cerebellum, and brainstem). FIG. 10E shows a comparison of the effects on SOD1 and CTNNB1 levels observed across all tested bis siRNA multi-targeted molecules, as well as a mixed siRNA control, broken out by location (right hemisphere of the brain at upper left, left hemisphere of the brain at upper right, cerebellum at lower right, and brainstem at lower left). A key noting reference identifiers and associated structures used in FIGS. 10D and 10E is also shown.

FIG. 11 shows degradation of bis-siRNA designs AM-183 to AM-190 in rat CSF after 0, 4, or 24 h incubation or 24 h incubation in PBS as a control.

FIG. 12 shows the nature of senses strand metabolites observed after 24 h incubation of bis-siRNA designs AM-183 to AM-190 in rat brain homogenate analyzed via MS, as described above.

FIG. 13 shows a schematic of the structure of parent SOD1-targeting siRNA AD-401824, noting the presence of SEQ ID NOs: 29 (top strand) and 31 (bottom strand).

FIGS. 14A and 14B present study design information for testing of a series of early bis-siRNA designs. FIG. 14A shows a study design for three differentially linked bis-siRNA designs, as compared to parent siRNA and an appropriate CSF control. FIG. 14B shows the structure of a “Q315” linker used in the AM-182 bis-siRNA design.

FIGS. 15A and 15B show results obtained for fluoro-linked bis-siRNA AM-178. FIG. 15A shows a schematic of the fluoro-linked AM-178 bis-siRNA design. FIG. 15B shows tissue distributions post-IT injection of the AM-178 bis-siRNA, as compared to parent siRNA AD-401824, at day 7 and day 28 timepoints.

FIGS. 16A and 16B show results obtained for DNA-linked bis-siRNA AM-181. FIG. 16A shows a schematic of the DNA-linked AM-181 bis-siRNA design. FIG. 16B shows tissue distributions post-IT injection of the AM-181 bis-siRNA, as compared to parent siRNA AD-401824, at day 7 and day 28 timepoints.

FIGS. 17A and 17B show results obtained for the 3×Q315-linked bis-siRNA AM-182. FIG. 17A shows a schematic of the 3×Q315-linked AM-182 bis-siRNA design. FIG. 17B shows tissue distributions post-IT injection of the AM-182 bis-siRNA, as compared to parent siRNA AD-401824, at day 7 and day 28 timepoints.

FIGS. 18A and 18B show delivered levels of parent siRNA and bis-siRNA designs in terminal CSF, assessed at day 7 and day 28. FIG. 18A shows results obtained for all tested animals. FIG. 18B shows a chart that has two animals removed, as compared to FIG. 18A above: animal #11 (day 7 AM-181) and #19 (day 28 AD-401824 parent siRNA). Notably, no CSF was obtained from animal #30, a 28 day AM-182-dosed animal, and no significant differences in CSF concentration of dosed agents was observed.

FIGS. 19A and 19B show observed levels of parent siRNA and bis-siRNA designs in plasma, assessed at day 7. FIG. 19A shows results obtained for all tested animals. FIG. 19B shows a chart that has an animal removed, as compared to FIG. 19A above: animal #11 (day 7 AM-181). Lower levels of AM-182 were specifically observed at day 7.

FIGS. 20A and 20B show observed levels of parent siRNA and bis-siRNA designs in plasma, assessed at day 28. Specifically, no significant differences were observed in long-term pharmacokinetics out to day 28. FIG. 20A shows results obtained for all tested animals. FIG. 20B shows a chart that has an animal removed (#19, AD-401824 day 28), as compared to FIG. 20A above.

FIGS. 21A and 21B show bis-sense strand quantification results. FIG. 21A shows that after IT injection, intact AM-178 and AM-181 bis-siRNAs were detected in plasma at 30 min post-dose. FIG. 21B shows that bis-sense strand quantification also revealed that intact AM-178, but not AM-181, was detected in CSF at day 7 and day 28.

FIGS. 22A-22H show the structure of exemplary CTNNB1(C16)-SOD1(C16) bis siRNA multi-targeted molecules having respective siRNA effector molecule sense strands joined by a three nucleotide DNA linker, as well as respective mSOD1 and mCTNNB1 levels of inhibition observed in mice injected with these constructs. All exemplary bis siRNA complexes tested are shown in FIG. 2A. FIG. 22A shows the sequence, structure and modification patterning of the SOD-targeting siRNA and the CTNNB1-targeting siRNA. The sequences are the same as shown in FIG. 1A, and the modification patterns are the same as those illustrated in FIG. 2B. FIG. 22B summarizes the configuration pattern and linkers used for each bis siRNA duplex of the exemplary bis-siRNA complexes used. FIG. 22C shows, for each individual mouse dosed, the percentage of SOD1 and CTNNB1 remaining at day 21 after a 100 μg ICV injection of each of the CTNNB1(C16)-SOD1(C16) bis siRNA molecules, in each of the indicated tissues (right hemisphere of the brain, left hemisphere of the brain, cerebellum, and brainstem), for a cohort of 4 animals. FIG. 22D shows the results of the percentage of SOD1 and CTNNB1, respectively, remaining at day 21 after a 100 μg ICV injection in the mice in each of the indicated tissues (right hemisphere of the brain, left hemisphere of the brain, cerebellum, and brainstem), for a cohort of 4 animals, comparing some CTNNB1(C16)-SOD1(C16) bis siRNA molecules (AM-183, AM-184, AM-185, and AM-186, as shown in FIG. 2A and Table 2) against the mixed duplex delivery (mixture of siRNA of AD-413709, targeting SOD1, and siRNA of AD-320650, targeting CTTNB1, as shown in FIG. 2A and Table 2). FIG. 22E shows the results of the percentage of SOD1 and CTNNB1, respectively, remaining at day 21 after a 100 μg ICV injection in the mice in each of the indicated tissues (right hemisphere of the brain, left hemisphere of the brain, cerebellum, and brainstem), for a cohort of 4 animals, comparing the bis siRNA complex possessing a single C₁₆ modification at SOD1-targeting siRNA (SOD1-C16) or CTNNB1-targeting siRNA (CTNNB1-C16) against the bis siRNA complex possessing a dual C₁₆ modification at both SOD1-targeting siRNA and CTNNB1-targeting siRNA (2C16 or CTNNB1(C16)-SOD1(C16)), and against the mixed duplex delivery (mixture of siRNA of AD-413709, targeting SOD1, and siRNA of AD-320650, targeting CTTNB1, as shown in FIG. 2A and Table 2). FIG. 22F shows the results of the percentage of SOD1 and CTNNB1, respectively, remaining at day 21 after a 100 μg ICV injection in the mice in each of the indicated tissues (right hemisphere of the brain, left hemisphere of the brain, cerebellum, and brainstem), for a cohort of 4 animals, comparing some various bis siRNA molecules (AM-183, AM-184, AM-188, and AM-189, as shown in FIG. 2A and Table 2) varying the positions of the respective siRNA effectors within the bis-siRNA complex. FIG. 22G shows, for each individual mouse dosed, the percentage of SOD1 and CTNNB1 remaining at day 21 after a 100 μg ICV injection of each of the CTNNB1(C16)-SOD1(C16) bis siRNA molecules, in the liver, for a cohort of 4 animals. FIG. 22H shows the percentage of SOD1 remaining at day 21 after an injection of a parent SOD1-targeting siRNA AD-401824 (Table 4) at various dosage (50 μg, 150 μg, or 300 μg), in the liver, for a cohort of 4 animals.

FIGS. 23A-23E show the structure of exemplary CTNNB1(C16)-SOD1(C16) bis siRNA multi-targeted molecules having respective siRNA effector molecule sense strands joined by a carbohydrate-based linker as compared to a nucleotide-based linker, as well as respective mSOD1 and mCTNNB1 levels of inhibition observed in mice injected with these constructs. The exemplary bis siRNA complexes tested are shown in FIG. 23A. There was also an exemplary circular bis-sciRNA (AM-206) illustrated in FIG. 23A. FIG. 23A summarizes the duplex ID, sense strand ID, tagert, and linker, as well as for the control mixed duplexes. Numbers of mice in tested cohorts, day of target inhibitory assessment, dose employed and locations of readouts obtained are also shown. All bis siRNA complexes included two sets of 21-mer sense strands and 23-mer antisense strands, wherein the sense strands of both siRNAs were made continuous via inclusion of a linker, while the respective antisense strands of siRNA duplexes were independent strands that were non-continuous. FIG. 23B shows the structures of various carbohydrate-based linkers in the exemplary bis-siRNA complexes, used in FIG. 23A. FIG. 23C summarizes the sequence, structure, configuration pattern, and linkers used for each bis siRNA duplex of the exemplary bis-siRNA complexes as well as an exemplary circular bis-sciRNA (AM-206) used in FIG. 23A. FIG. 23D shows, for each individual mouse dosed, the percentage of SOD1 and CTNNB1 remaining at day 21 after a 100 μg ICV injection of each of the CTNNB1(C16)-SOD1(C16) bis siRNA molecules as well as an exemplary circular bis-sciRNA (AM-206), in the brain, for a cohort of 4 animals, comparing the bis siRNA complex possessing a three-carbohydrate linker (AM-203, AM204, AM205) against the bis siRNA complex possessing a three-nucleotide linker (AM183, AM202), and against the mixed duplex delivery (mixture of siRNA of AD-401824, targeting SOD1, and siRNA of AD-503801, targeting CTTNB1, as shown in FIG. 23C). FIG. 23E shows, for each individual mouse dosed, the percentage of SOD1 and CTNNB1 remaining at day 21 after a 100 μg ICV injection of each of the CTNNB1(C16)-SOD1(C16) bis siRNA molecules as well as an exemplary circular bis-sciRNA (AM-206), in each of the indicated tissues (liver, heart), for a cohort of 4 animals, comparing the bis siRNA complex possessing a three-carbohydrate linker (AM-203, AM204, AM205) against the bis siRNA complex possessing a three-nucleotide linker (AM183, AM202), and against the mixed duplex delivery (mixture of siRNA of AD-401824, targeting SOD1, and siRNA of AD-503801, targeting CTTNB1, as shown in FIG. 23C).

FIGS. 24A-24D show the structure of exemplary CTNNB1(C16)-SOD1(C16) bis siRNA multi-targeted molecules having respective siRNA effector molecule sense strands joined by various linkers and having various chemical modifications in the bis-siRNAs, as well as respective mSOD1 and mCTNNB1 levels of inhibition observed in mice injected with these constructs. The exemplary bis siRNA complexes tested are shown in FIG. 24A. There was also an exemplary circular bis-sciRNA (AM-206) illustrated in FIG. 24A. FIG. 24A summarizes the duplex ID, linker and chemistries, as well as for the control mixed duplexes. Numbers of rats in tested cohorts, duration of target inhibitory assessment, dose employed and locations of readouts obtained are also shown. All bis siRNA complexes included two sets of 21-mer sense strands and 23-mer antisense strands, wherein the sense strands of both siRNAs were made continuous via inclusion of a three-nucleotide single-stranded linker, while the respective antisense strands of siRNA duplexes were independent strands that were non-continuous. FIG. 24B shows the structures of various linkers in the exemplary bis-siRNA complexes and the exemplary circular bis-sciRNA, used in FIG. 24A. FIG. 24C summarizes the sequence, structure, configuration pattern, and linkers used for each bis siRNA duplex of the exemplary bis-siRNA complexes used in FIG. 24A. FIG. 24D shows, for each individual mouse dosed, the percentage of SOD1 and CTNNB1 remaining at day 15 and day 29, respectively, after an intrathecal (IT) dosing (at 0.3 mg) of each of the CTNNB1(C16)-SOD1(C16) bis siRNA molecules (containing various linkers joining the respective sense strands of the individual effector molecules (siRNAs) and various chemical modifications in the bis-siRNA molecules, as shown in FIG. 24A-24C) as well as an exemplary circular bis-sciRNA (AM-206) was performed at to, in each of the indicated tissues (thoracic spinal cord, frontal cortex, hippocampus, and striatum), for a cohort of 4 animals, comparing against the mixed duplex delivery (mixture of siRNA of AD-401824, targeting SOD1, and siRNA of AD-503801, targeting CTTNB1, as shown in FIG. 23C).

FIGS. 25A-25D show the structure of exemplary CTNNB1(C16)-SOD1(C16) bis siRNA multi-targeted molecules having respective siRNA effector molecule sense strands joined by various linkers, as well as respective mSOD1 and mCTNNB1 levels of inhibition observed in mice injected with these constructs. The exemplary bis siRNA complexes tested are shown in FIG. 25A. FIG. 25A summarizes the duplex ID, linker chemistries, as well as for the control mixed duplexes. Numbers of rats in tested cohorts, duration of target inhibitory assessment, dose employed and locations of readouts obtained are also shown. All bis siRNA complexes included two sets of 21-mer sense strands and 23-mer antisense strands, wherein the sense strands of both siRNAs were made continuous via inclusion of a three-nucleotide single-stranded linker, while the respective antisense strands of siRNA duplexes were independent strands that were non-continuous. FIG. 25B shows the structures of various linkers in the exemplary bis-siRNA complexes, used in FIG. 25A. FIG. 25C summarizes the sequence, structure, configuration pattern, and linkers used for each bis siRNA duplex of the exemplary bis-siRNA complexes used in FIG. 25A. FIG. 25D shows, for each individual mouse dosed, the percentage of SOD1 and CTNNB1 remaining at day 15, after an intrathecal (IT) dosing (at 0.6 mg) of each of the CTNNB1(C16)-SOD1(C16) bis siRNA molecules (containing various linkers joining the respective sense strands of the individual effector molecules (siRNAs), as shown in FIG. 25A-25C) was performed at to, in each of the indicated tissues (thoracic spinal cord, cerebellum, frontal cortex, hippocampus, and striatum), for a cohort of 4 animals, comparing against the mixed duplex delivery (mixture of siRNA of AD-401824, targeting SOD1, and siRNA of AD-503801, targeting CTTNB1, as shown in FIG. 23C).

FIGS. 26A-26D are schematic summaries showing the in vivo stability of the exemplary bis-siRNAs and/or circular bis-sciRNA after incubation of the molecules in rat brain homogenate measured using LC-MS. FIG. 26A shows the stability of the exemplary bis-siRNAs (AM-183 to AM-186) with different linker chemistries in rat brain homogenate. FIG. 26B shows the stability of the exemplary bis-siRNAs (AM-183, AM-184, AM-188, and AM-189) with varying orientation chemsitry in rat brain homogenate. FIG. 26C shows the stability of the exemplary bis-siRNAs possessing a single C₁₆ modification at SOD1-targeting siRNA or CTNNB1-targeting siRNA (AM-190 and AM-187) and the bis siRNA complex possessing a dual C₁₆ modification at both SOD1-targeting siRNA and CTNNB1-targeting siRNA (AM-183) in rat brain homogenate, compared against the mixture of duplexes (AD-320650 and AD-413709). FIG. 26D shows the metabolic liabilities of the exemplary bis-siRNAs and exmplary circular bis-sciRNA in rat brain homogenate for CNS-targeting (AM-183, AM-202, AM-203, AM-204, AM-205, and AM-206) and for liver-targeting (AM-191, AM-207, AM-208, AM-209, AM-210, and AM-211).

FIG. 27A is a schematic representation of an exemplary GalNAc-sciRNA duplex. FIG. 27B illustrates the chemical modifications used in the exemplary GalNAc-sciRNA duplex.

FIGS. 28A-28D are graphs of decay curves of enzymatic digestion using single-stranded poly 2′-deoxy linear and circular oligonucleotides (ON-3 and ON-4, respectively) and single-stranded fully 2′-modified linear and circular oligonucleotides (ON-5 and ON-6, respectively) in an in vitro assay using either a 3′-exonuclease (FIG. 28A and FIG. 28B) or 5′-exonuclease (FIG. 28C and FIG. 28D).

FIGS. 29A-29B are graphs showing the stability of full-length sense strand after incubation of the duplex in plasma and liver homogenate measured using LC-MS. FIG. 29A shows the mean natural logarithm of the percent of sense strand remaining in rat plasma. FIG. 29B shows the mean natural logarithm of the percent of sense strand remaining in rat liver homogenate. Plotted are means. Error bars are standard deviation of three replicates per time point.

FIG. 30 is a graph showing imino regions of 1D ¹H NMR spectra of linear structure GalNAc-siRNAs (Table 9, si-1, si-2, si-3 and si-6) and cyclic structure GalNAc-sciRNA duplexes (Table 9, si-4 and si-5). The imino protons engaged in Watson-Crick base pairs display chemical shift values in the range from δ 12 to 14 ppm.

FIGS. 31A-31B are graphs of pharmacodynamics profiles after a single subcutaneous administration of linear GalNAc-siRNA (Table 9, si-1, si-2 and si-6) and circular GalNAc-sciRNA (Table 9, si-4, si-5 and si-7) conjugates in mice. A single dose of each conjugate (3 mg/kg) was administered in mice on Day 0, and serum was collected on Days 0 (pre-dose), 3, 7 and 14. Circulating serum protein levels for TTR (FIG. 31A) and C5 (FIG. 31B) were determined using an appropriate ELISA kit, relative to PBS groups. Error bars are SD (n=3).

FIGS. 32A-32B are graphs showing the whole liver and Ago2 levels of antisense strand for linear GalNAc-siRNA (Table 9, si-1, si-2 and si-3) and circular GalNAc-sciRNA (Table 9, si-4 and si-5) conjugates in mice. FIG. 32A shows the liver levels of antisense strands isolated and measured from whole mouse livers. FIG. 32B shows the levels of antisense strand isolated and measured from immunoprecipitated Ago2 from whole mouse livers. A single dose of each conjugate (3 mg/kg) was administered in mice on Day 0, and livers were collected on Day 7. Levels were determined using SL-RT QPCR relative to PBS groups. Error bars are SD (n=3).

FIG. 33 illustrates a model of a sciRNA:Ago2 complex based on the crystal structure of Ago2 bound to duplex RNA with seed region pairing. Z linker carbons are highlighted. Selected side chains of the Ago2 PIWI and MID domains and L2 linker region are labeled. The view is across the major (top) and minor grooves (bottom) of the seed region duplex.

### DETAILED DESCRIPTION

The present disclosure is based, at least in part, upon discovery of molecules that target more than one target nucleic acid and that exhibit robust and surprising levels of efficacy in the tissues of the CNS of a subject following CNS-directed administration of such multi-targeted molecules. CNS-directed delivery and efficacy of such multi-targeted molecules was herein identified as robust when each effector molecule of a multi-targeted molecule included at least one lipophilic moiety, with delivery and efficacy observed to be significantly reduced in CNS tissues for multi-targeted molecules not harboring at least one lipophilic moiety conjugated to each effector molecule. Pharmaceutical compositions, injectates, methods (including therapeutic methods), and other related aspects are also described herein.

Bis siRNA Compounds (Two Effector Molecules Connected by a Bis-Linker)

In one aspect, provided herein are multi-targeted molecules that are based on bis siRNA compounds.

Generally, the multi-targeted molecules comprise at least two nucleic acid-based effector molecules, wherein said at least two nucleic acid-based effector molecules are covalently or non-covalently linked to each other. Without limitations, any nucleic acid-based effector molecule capable of modulating gene expression of a target can be comprised in the multi-targeted molecules disclosed herein.

The multi-targeted molecules include at least two nucleic acid-based effector molecules that are linked to each other by a linker moiety (e.g., a nucleic acid sequence, one or more carbohydrate moieties, or other organic polymer, optionally including cleavable forms of such linker moieties) as described herein. Each of the at least two nucleic acid-based effector molecules of the multi-targeted molecule harbors a lipophilic ligand (e.g., a saturated or unsaturated C₁₆ hydrocarbon chain), which promotes effective CNS targeting of each molecular target of the multi-targeted molecule. Without limitations, any nucleic acid-based effector molecule capable of modulating gene expression of a target can be included in the multi-targeted molecules disclosed herein.

Thus, in certain aspects, the instant disclosure provides a multi-targeted molecule for modulating in the central nervous system (CNS) of a subject one or more distinct target RNA sequences in one or more target RNAs in the central nervous system (CNS) of a subject, the multi-targeted molecule including at least two nucleic acid-based effector molecules, where the effector molecules are connected together by a linker and do not overlap with each other, where each of the at least two effector molecules has at least one conjugated lipophilic moiety, and where the multi-targeted molecule delivers to the central nervous system (CNS) of the subject and is capable of inhibiting the activity or expression of the one or more target RNAs in a tissue of the CNS of the subject by at least 15% each, relative to an appropriate control.

Two target RNA sequences within a single target RNA are considered “distinct” when the target RNA sequences do not overlap with each other.

In certain embodiments, both of the two nucleic acid-based effector molecules target the same target RNA sequence. In such embodiments, the multi-targeted molecule may have a “symmetric” design (e.g., the linker may connect the sense strand 3′ ends or 5′ ends of two identical siRNAs). In certain other embodiments, the two nucleic acid-based effector molecules target different target RNA sequences. In such embodiments, the multi-targeted molecule has an “asymmetric” design. In the latter case, a design may also be “asymmetric” when both nucleic acid-based effector molecules target the same target RNA, but at “distinct” target RNA sequences.

“Appropriate control” as used herein refers to either a composition otherwise identical to the composition comprising the relevant active agents, but lacking such active agents; or a composition comprising an active agent (e.g., oligonucleotide) that is not targeted to the relevant target nucleic acid(s). An otherwise identical composition that lacks an active agent can include, for example, a buffer solution used for parenteral administration, such as phosphate-buffered saline (PBS) or artificial cerebrospinal fluid (aCSF). aCSF can comprise a sterile aqueous composition having a pH of about 7.2 and the following ion concentrations (in mM): Na⁺ 150; K⁺ 3.0; Ca²⁺ 1.4; Mg²⁺ 0.8; P 1.0; and Cl⁻ 155. An oligonucleotide that is not targeted to the relevant target nucleic acid(s) can include, for example, a polyadenoside-based oligonucleotide, such as AD-77748 (see Tables 2 and 3).

By a “nucleic acid-based effector molecule” is meant a modified or unmodified single-stranded or double-stranded nucleic acid molecule capable of modulating the activity of expression of a target nucleic acid. In some embodiments, a nucleic acid-based effector molecule is a modified or unmodified single-stranded or double-stranded nucleic acid molecule capable of modulating the gene expression of a target gene. Exemplary nucleic acid-based effector molecules capable of modulating gene expression of a target gene include, but are not limited to, double-stranded and single-stranded RNA interference agents (such as siRNA and shRNA, and also referred to as dsRNA agents herein), ribozymes, triplex-forming oligonucleotides, decoy oligonucleotides, immunostimulatory oligonucleotides, RNA activators, U1 adaptors, guide RNA (gRNA) of CRISPR Cas, combinations thereof, and the like. In certain embodiments, each single-stranded or double-stranded nucleic acid molecule of the effector molecule contains at least one modified nucleotide or at least one modified internucleotide linkage.

It is noted that the at least two effector molecules are two separate effector molecules. In other words, the at least two effector molecules do not overlap with each other. As such, the multi-targeted molecules disclosed herein differ from molecules wherein one effector molecule is directed to two different targets, for example, double-stranded effector molecules wherein each strand is directed to a different target or an effector molecule comprising a sequence, wherein at least a portion of the sequence is complementary to or can hybridize with two different target sequences.

In some embodiments, the multi-targeted molecule is assembled from two separate siRNA molecules, wherein at least one of the siRNAs has at least one ligand attached thereto. In some other embodiments, the multi-targeted molecule is assembled from two separate siRNA molecules, wherein each siRNA has at least one ligand attached thereto.

In various embodiments of the multi-targeted molecule, where at least two siRNAs, each having at least one ligand, are linked to each other, said at least two ligands can be the same or they can be different. Further, the said at least ligands can be conjugated independently at any position of the respective siRNAs. For example, one ligand can be attached to the sense strand of the first siRNA and the other can be attached to the sense strand of the second siRNA, or one ligand can be attached to the sense strand of the first siRNA and the other can be attached to the antisense strand of the second siRNA, or one ligand can be attached to the antisense strand of the first siRNA and the other can be attached to the antisense strand of the second siRNA. Without limitations, the first ligand can be attached independently at the 5′-end, 3′-end or at an internal (non-terminal) position of one strand (sense or antisense) of the first siRNA. Similarly, the second ligand can be attached independently at the 5′-end, 3′-end or at an internal (non-terminal) position of one strand (sense or antisense) of the second siRNA.

In some embodiments, one ligand is conjugated to 3′-end of a sense strand of the first siRNA and the other ligand is conjugated to the 3′-end of an antisense strand of the second siRNA.

In some embodiments, one ligand is conjugated to 5′-end of a sense strand of the first siRNA and the other ligand is conjugated to the 3′-end of an antisense strand of the second siRNA. In some embodiments, one ligand is conjugated to 3′-end of a sense strand of the first siRNA and the other ligand is conjugated to the 5′-end of an antisense strand of the second siRNA. In some embodiments, one ligand is conjugated to 5′-end of a sense strand of the first siRNA and the other ligand is conjugated to the 5′-end of an antisense strand of the second siRNA. In some embodiments, one ligand is conjugated to 3′-end of a sense strand first siRNA and the other ligand is conjugated at an internal (non-terminal) position of an antisense strand of the second siRNA. In some embodiments, one ligand is conjugated to 5′-end of a sense strand of the first siRNA and the other ligand is conjugated at an internal (non-terminal) position of an antisense strand of the second siRNA. In some embodiments, one ligand is conjugated to 3′-end of an antisense strand of the first siRNA and the other ligand is conjugated at an internal (non-terminal) position of a sense strand of the second siRNA. In some embodiments, one ligand is conjugated to 5′-end of an antisense strand of the first siRNA and the other ligand is conjugated at an internal (non-terminal) position of a sense strand of the second siRNA. In some embodiments, one ligand is conjugated at an internal (non-terminal) position of an antisense strand of the first siRNA and the other ligand is conjugated at an internal (non-terminal) position of a sense strand of the second siRNA.

In some embodiments, one ligand is conjugated to 3′-end of a first sense strand and the other ligand is conjugated to the 3′-end of a second sense strand. In some embodiments, one ligand is conjugated to 3′-end of a first sense strand and the other ligand is conjugated to the 5′-end of a second sense strand. In some embodiments, one ligand is conjugated to 5′-end of a first sense strand and the other ligand is conjugated to the 3′-end of a second sense strand. In some embodiments, one ligand is conjugated to 5′-end of a first sense strand and the other ligand is conjugated to the 5′-end of a second sense strand. In some embodiments, one ligand is conjugated to 3′-end of a first sense strand and the other ligand is conjugated at an internal (non-terminal) position of a second sense strand. In some embodiments, one ligand is conjugated to 5′-end of a first sense strand and the other ligand is conjugated to an internal (non-terminal) position of a second sense strand. In some embodiments, one ligand is conjugated at an internal (non-terminal) position of a first sense strand and the other ligand is conjugated at an internal (non-terminal) position of a second sense strand. In some embodiments, one ligand is conjugated to 3′-end of a first antisense strand and the other ligand is conjugated to the 3′-end of a second antisense strand. In some embodiments, one ligand is conjugated to 3′-end of a first antisense strand and the other ligand is conjugated to the 5′-end of a second antisense strand. In some embodiments, one ligand is conjugated to 5′-end of a first antisense strand and the other ligand is conjugated to the 3′-end of a second antisense strand. In some embodiments, one ligand is conjugated to 5′-end of a first antisense strand and the other ligand is conjugated to the 5′-end of a second antisense strand. In some embodiments, one ligand is conjugated to 3′-end of a first antisense strand and the other ligand is conjugated at an internal (non-terminal) position of a second antisense strand. In some embodiments, one ligand is conjugated to 5′-end of a first antisense strand and the other ligand is conjugated to an internal (non-terminal) position of a second antisense strand. In some embodiments, one ligand is conjugated at an internal (non-terminal) position of a first antisense strand and the other ligand is conjugated at an internal (non-terminal) position of a second antisense strand.

In some embodiments, the multi-targeted molecule is assembled from two siRNAs wherein sense strand of the first siRNA is covalently linked to the sense strand of the second siRNA. Without limitations, the two sense strands can be linked to each other in any orientation. For example, 3′-end of the first sense strand can be linked to 5′-end of the second sense strand; 3′-end of the first sense strand can be linked to 3′-end of the second sense strand; or 5′-end of the first sense strand can be linked to 5′-end of the second sense strand.

In some embodiments, the multi-targeted molecule is assembled from two siRNAs wherein antisense strand of the first siRNA is covalently linked to the antisense strand of the second siRNA. Without limitations, the two antisense strands can be linked to each other in any orientation. For example, 3′-end of the first antisense strand can be linked to 5′-end of the second antisense strand; 3′-end of the first antisense strand can be linked to 3′-end of the second antisense strand; or 5′-end of the first antisense strand can be linked to 5′-end of the second antisense strand.

In some embodiments, the multi-targeted molecule is assembled from two siRNAs wherein sense strand of the first siRNA is covalently linked to the antisense strand of the second siRNA. Without limitations, the sense strand of the first siRNA can be linked to the antisense strand of the second siRNA in any orientation. For example, 3′-end of the sense strand can be linked to 5′-end of the antisense strand; 3′-end of the sense strand can be linked to 3′-end of the antisense strand; or 5′-end of the sense strand can be linked to 5′-end of the antisense strand.

In some embodiments, the multi-targeted molecule modulates two or more distinct target RNAs in the central nervous system (CNS), and the multi-targeted molecule is assembled from two double-stranded RNAs (dsRNA) targeting two or more distinct target RNAs, and the orientation of the two dsRNA with respect to the linker connecting them may vary.

In some embodiments, the multi-targeted molecule is assembled from two dsRNAs according to the formula:

dsRNA1-L-dsRNA2,

wherein dsRNA1 is the first dsRNA targeting a first target RNA sequence, dsRNA2 is the second dsRNA targeting a second, different target RNA sequence, and L is the linker connecting dsRNA1 to dsRNA2. L connects 3′ end of the sense strand of dsRNA1 to dsRNA2, and/or 5′ end of the antisense strand of dsRNA1 to dsRNA2.

In one embodiment, the multi-targeted molecule is represented by

| | | | | |
|---|---|---|---|---|
| 5′ | ss1 | - L - | ss2 | 3′ |
| 3′ | as1 | | as2 | 5′, |

wherein ss1 is the sense strand of dsRNA1, as1 is the antisense strand of dsRNA1, ss2 is the sense strand of dsRNA2; as2 is the antisense strand of dsRNA2, wherein L connects the 3′-end of ss1 to 5′-end of ss2.

In some embodiments, the multi-targeted molecule is assembled from two dsRNAs according to the formula:

dsRNA2-L-dsRNA1,

wherein dsRNA1 is the first dsRNA targeting a first target RNA sequence, dsRNA2 is the second dsRNA targeting a second, different target RNA sequence, and L is the linker connecting dsRNA2 to dsRNA1. L connects 3′ end of the sense strand of dsRNA2 to dsRNA1, and/or 5′ end of the antisense strand of dsRNA2 to dsRNA1.

In one embodiment, the multi-targeted molecule is represented by

| | | | | |
|---|---|---|---|---|
| 5′ | ss2 | - L - | ss1 | 3′ |
| 3′ | as2 | | as1 | 5′, |

wherein ss2 is the sense strand of dsRNA2, as2 is the antisense strand of dsRNA2, ss1 is the sense strand of dsRNA1; as1 is the antisense strand of dsRNA1, wherein L connects the 3′-end of ss2 to 5′-end of ss1.

In some embodiments, the multi-targeted molecule is assembled from two siRNAs wherein sense strand of the first siRNA is covalently linked to the sense strand of the second siRNA and antisense strand of the first siRNA is covalently linked to the antisense strand of the second siRNA

In some embodiments, the multi-targeted molecule is assembled from two siRNAs wherein antisense strand of the first siRNA is covalently linked to the sense strand of the second siRNA and sense strand of the first siRNA is covalently linked to the antisense strand of the second siRNA.

In some embodiments, at least one of the effector molecules in the multi-targeted molecules disclosed herein is a ribozyme. In some embodiments, the multi-targeted molecule comprises at least two ribozymes. Without limitations, the ribozymes can be same or different.

In some embodiments, at least one of the effector molecules in the multi-targeted molecules disclosed herein is a siRNA and at least one of the effector molecules is a ribozyme.

In some embodiments, at least one of the effector molecules in the multi-targeted molecules disclosed herein is an aptamer. In some embodiments, the multi-targeted molecule comprises at least two aptamers. Without limitations, the aptamers can be same or different.

In some embodiments, at least one of the effector molecules in the multi-targeted molecules disclosed herein is a siRNA and at least one of the effector molecules is an aptamer.

In some embodiments, at least one of the effector molecules in the multi-targeted molecules disclosed herein is a decoy oligonucleotide. In some embodiments, the multi-targeted molecule comprises at least two decoy oligonucleotides. Without limitations, the decoy oligonucleotides can be same or different.

In some embodiments, at least one of the effector molecules in the multi-targeted molecules disclosed herein is a siRNA and at least one of the effector molecules is a decoy oligonucleotide.

In some embodiments, at least one of the effector molecules in the multi-targeted molecules disclosed herein is a U1 adaptor. In some embodiments, the multi-targeted molecule comprises at least two U1 adaptors. Without limitations, the U1 adaptors can be same or different.

In some embodiments, at least one of the effector molecules in the multi-targeted molecules disclosed herein is a siRNA and at least one of the effector molecules is a U1 adaptor.

In some embodiments, at least one of the effector molecules in the multi-targeted molecules disclosed herein is an activating RNA. In some embodiments, the multi-targeted molecule comprises at least two activating RNAs. Without limitations, the activating RNAs can be same or different.

In some embodiments, at least one of the effector molecules in the multi-targeted molecules disclosed herein is a siRNA and at least one of the effector molecules is an activating RNA.

In some embodiments, at least one of the effector molecules in the multi-targeted molecules disclosed herein is a triplex forming oligonucleotide. In some embodiments, the multi-targeted molecule comprises at least two triplex forming oligonucleotides. Without limitations, the Triplex forming oligonucleotides can be same or different.

In some embodiments, at least one of the effector molecules in the multi-targeted molecules disclosed herein is a siRNA and at least one of the effector molecules is a triplex forming oligonucleotide.

### Lipophilic Moieties

Certain aspects of the instant disclosure feature lipophilic moieties conjugated to the multi-targeted molecules. Lipophilic moieties/ligands have been identified as particularly useful for achieving CNS delivery and target RNA knockdown efficacy in the CNS for nucleic acid therapeutics. Exemplary lipophilic moieties for use herein include, without limitation, lipids, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, and phenoxazine.

A lipophilic moiety can include a saturated or unsaturated C₄-C₃₀ hydrocarbon chain, as well as an optional functional group such as hydroxyl, amine, carboxylic acid, sulfonate, phosphate, thiol, azide, or alkyne. Optionally, a lipophilic moiety of the instant disclosure can include a saturated or unsaturated C₆-C₁₈ hydrocarbon chain. Optionally, a lipophilic moiety of the instant disclosure can include a saturated or unsaturated C₁₆ hydrocarbon chain.

In certain embodiments, each lipophilic moiety can be independently selected from a saturated or unsaturated C₆, C₈, C₁₀, C₁₂, C₁₄, C₁₆, C₁₈, C₂₀, and C₂₂ hydrocarbon chain.

In certain embodiments, each lipophilic moiety can be independently selected from a linear and saturated or unsaturated C₆, C₈, C₁₀, C₁₂, C₁₄, C₁₆, C₁₈, C₂₀, and C₂₂ hydrocarbon chain.

In certain embodiments, each lipophilic moiety can be independently selected from a linear and saturated C₆, C₈, C₁₀, C₁₂, C₁₄, C₁₆, C₁₈, C₂₀, and C₂₂ hydrocarbon chain.

As described herein, lipophilic moieties can be conjugated to the multi-targeted molecules of the instant disclosure via a monovalent or branched bivalent or trivalent linker.

In embodiments, at least one lipophilic moiety is conjugated to the multi-targeted molecule through a monovalent or branched bivalent or trivalent linker.

Certain embodiments feature the following C₁₆ hydrocarbon chain molecule as a specifically exemplified form of lipophilic molecule:

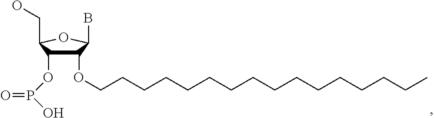

where B is a nucleotide base or a nucleotide base analog, optionally where B is adenine, guanine, cytosine, thymine or uracil.

### Linkers

In certain embodiments, linkers are employed to connect the effector molecules of a multi-targeted molecule of the instant disclosure. A range of specifically contemplated linkers are available for conjugating the effector molecules of the multi-targeted molecules of the instant disclosure, including, without limitation, DNA, RNA, disulfide, amide, functionalized monosaccharides or oligosaccharides of galactosamine, glucosamine, glucose, galactose, mannose, other organic polymer linkers, and combinations thereof.

In some embodiments, at least two nucleic acid based effector molecules in the multi-targeted molecules of the instant disclosure can be covalently linked to each other via nucleotide-based linkers or non-nucleotide based linkers as generally known in the art (refer, e.g., to WO 2017/05109 and WO 2018/136620, each of which is incorporated by this reference in its entirety) and as described herein. Accordingly, in some embodiments, at least two effector molecules in the multi-targeted molecule are linked via a nucleotide-based linker. In some other embodiments, at least two effector molecules are linked via a non-nucleotide based linker.

It is noted that a nucleotide-based linker may form part of one or both the effector molecules being connected together. What is meant by this is that at least a portion of the nucleotide sequence of the linker is needed for functioning of one of the effector molecules. In certain embodiments, the nucleotide sequence of the linker does not form part of the effector molecule. In other words, either of the effector molecules does not require any part of the nucleotide sequence of the linker to modulate gene expression. For example, if the linker sequence is removed from the effector molecule, the effector molecule is still capable of modulating gene expression at a similar level (e.g., within 95%) relative to when the linker is present. Where the effector molecule needs complementarity with the target gene for activity, the linker may or may not be part of the effector molecule needed for complementarity to the target sequence. In some embodiments, the linker does not have complementarity (e.g., less than 5% complementarity) with or hybridize to the target sequence.

For nucleic acid linkers, oligonucleotides of any length and modification pattern can be employed, with optional exemplary linker length including, without limitation, between one and 30 nucleotides in length. Optionally, the linker length is between two and 20 nucleotides, optionally between two and fifteen nucleotides, optionally between two and ten nucleotides, optionally between two and five nucleotides, optionally two, three or four nucleotides in length.

A nucleotide linker can be single-stranded or double-stranded. In some embodiments, a first strand of a double-stranded nucleotide-based linker connecting the two effector molecules comprises a nucleotide sequence substantially complementary to the second strand of said double-stranded nucleotide-based linker. In some embodiments, the first strand of the linker comprises a nucleobase sequence that is at least 75% (e.g., 75%, 80%, 85%, 90%, 95% or more) complementary to the nucleobase sequence of the second strand of the linker. In some embodiments, the first strand of the linker comprises a nucleobase sequence that is fully complementary to the nucleobase sequence of the second strand of the linker connecting the two effector molecules.

Without limitation, a nucleotide-based linker connecting the effector molecules can be all DNA, all RNA or a mixture of DNA and RNA. In some embodiments, the nucleotide-based linker connecting the two effector molecules is all DNA. The RNA and DNA can be natural and modified. Accordingly, in some embodiments, the nucleotide-based linker connecting the effector molecules comprises at least one modification selected from among the following: a modified internucleoside linkage, a modified nucleobase, a modified sugar, and any combinations thereof. Exemplary modifications for the linker include, but are not limited to, locked nucleic acids (e.g., LNA, ENA and BNA), 2′-O-alkyl nucleosides, 2′-halo nucloesides (such as 2′-F nucleotides), 2′-amino nucleosides, 2′-S-alkyl nucleosides, abasic nucleosides, 2′-cyano nucleosides, 2′-mercapto nucleosides; 2′-MOE nucleosides, acyclic nucleosides, (S)-cEt monomers, and modified internucleotide linkages (such as phosphodiesters, phosphotriesters, hydrogen phosphonates, alkyl or aryl phosphonates, phosphoramidates, phosphorothioates, phosphorodithioates, methylenemethylimino, thiodiester, thionocarbamate, N,N′-dimethylhydrazine, phosphoroselenates, borano phosphates, borano phosphate esters, amides, hydroxylamino, siloxane, dialkylsiloxane, carboxamide, carbonate, carboxymethyl, carbamate, carboxylate ester, thioether, ethylene oxide linker, sulfide, sulfonate, sulfonamide, sulfonate ester, thioformacetal, formacetal, oxime, methyleneimino, methykenecarbonylamino, methylenemethylimino, methylenehydrazo, methylenedimethylhydrazo, methyleneoxymethylimino, ethers, thioethers, and thioacetamido). Such modifications can also be present upon non-linker elements of the multi-targeted molecules of the instant disclosure. Nucleic acid modifications are described in more detail elsewhere in the instant disclosure.

In some embodiments, at least one of the internucleoside linkages between the linker connecting the effector molecules and an effector molecule is a modified internucleoside linkage. In some embodiments, the internucleoside linkage connecting the 5′-end of the linker to the 3′-end of one of the effector molecule is a modified internucleoside linkage. In some embodiments, the internucleoside linkage connecting the 3′-end of the linker to the 5′-end of one of the effector molecules is a modified internucleoside linkage.

In some embodiments, first (e.g., first, second, third, fourth or fifth) internucleoside linkage at the 5′- and/or 3′-end of the linker connecting the effector molecules is a modified internucleoside linkage. In some embodiments, one, two, three, four, five or more internucleoside linkages from the 5′- and/or 3′-end of the linker are modified internucleoside linkages.

In some embodiments, the linker connecting the effector molecules comprises at least one (e.g., one, two, three, four, five, six or more) modified internucleoside linkages at an internal (non-terminal) position of the linker.

Without limitations, the nucleotide-based linker connecting the effector molecules can be of any desired length. For example, the nucleotide-based linker connecting the effector molecules can be 1, 2, 3, 4, 5, 6, 7, 8, 9, 10, 11, 12, 13, 14, 15, 16, 17, 18, 19, 20 or more nucleotides in length. In some embodiments, the nucleotide-based linker connecting the effector molecules can range in length from 1 nucleotide to 5 nucleotides in length. In a particular embodiment, the nucleotide-based linker connecting the two effector molecules is 4 nucleotides in length.

When the nucleotide-based linker connecting the effector molecules comprises a nucleic acid modification, such modification can be located at any position in the linker. For example, the modification can be at the 5′-nucleotide, the 3′-nucleotide or at an internal (non-terminal) nucleotide of the linker. In some embodiments, first (e.g., first, second, third, fourth or fifth) nucleotide at the 5′- and/or 3′-end of the linker comprises a nucleic acid modification. In some embodiments, one, two, three, four, five or more nucleotides from the 5′- and/or 3′-end of the linker comprise a nucleic acid modification. In some embodiments, one, two, three, four, five or more internal (non-terminal) nucleotides of the linker comprise a nucleic acid modification. In some embodiments, internal (non-terminal) nucleotides of the linker comprise all DNA on the sense strand. In another embodiment, the internal (non-terminal) nucleotides of the linker comprise a mixture of DNA and 2′-OAlkyl modifications on the antisense strand.

The nucleotide-based linker connecting the effector molecules can comprise one or two nucleic acid strands and can be single stranded, double-stranded, or comprise single-stranded and double-stranded regions. In some embodiments, the nucleotide-based linker connecting the effector molecules comprises two nucleic acid strands that do not form a double-stranded structure. In other words, the nucleotide-based linker comprises two strands that do not hybridize with each other.

In some embodiments, the nucleotide-based linker connecting the effector molecules comprises two nucleic acid strands, wherein nucleotide sequence of the first strand of the linker comprises at least one (e.g., one, two, three, four, five or more) nucleotide mismatch with the nucleotide sequence of the second strand of the linker. In some embodiments, at least one of the strands of the linker comprises a bulge or a loop. For example, at least one of the linker strands comprises at least one (e.g., one, two, three, four, five or more consecutive or nonconsecutive) non-complementary nucleobase with the other linker strand.

Without limitations, the nucleotide-based linker connecting the effector molecules can comprise one or more nucleic acid modifications disclosed herein. When the nucleotide-based linker connecting the effector molecules comprises two nucleic acid strands, each strand can be independently unmodified or comprise one or more nucleic acid modifications disclosed herein. Accordingly, in some embodiments, the nucleotide-based linker connecting the effector molecules comprises two nucleic acid strands where each strand is unmodified. In some embodiments, the nucleotide-based linker connecting the effector molecules comprises two nucleic acid strands, wherein one strand is unmodified and the other strand comprises at least one modification selected from the group consisting of modified internucleoside linkage, modified nucleobase, modified sugar, and any combinations thereof. In some embodiments, the nucleotide-based linker connecting the effector molecules comprises two nucleic acid strands and both strands comprise at least one modification independently selected from the group consisting of modified internucleoside linkage, modified nucleobase, modified sugar, and any combinations thereof.

In some embodiments, the nucleotide-based linker connecting the effector molecules comprises two nucleic acid strands and wherein one of the strands comprises all DNA and the other strand comprises a mixture of DNA and 2′-Oalkyl modifications.

The nucleotide-based linker connecting the effector molecules can be resistant to degradation or cleavage by a single- or double-strand nuclease. Alternatively, a nucleotide-based linker connecting the effector molecules can be a cleavable linker. For example, a linker connecting the effector molecules can undergo cleavage by a single- or double-strand nuclease.

As described herein, the linker connecting the effector molecules in a multi-targeted molecule can be a non-nucleotide based linker. In some embodiments, the non-nucleotide based linker connecting the two oligonucleotides comprises a cleavable group.

In some embodiments, the non-nucleotide based linker connecting the two oligonucleotides comprises at least one disulfide group.

In certain embodiments, at least two effector molecules in the multi-targeted molecule are covalently linked to each other via a nucleotide-based or non-nucleotide based linker and the multi-targeted molecule is further conjugated with at least one ligand. Without limitations, the ligand can be present anywhere in the multi-targeted molecule. For example, the ligand can be present at one end of one of the at least two effector molecules covalently linked by the linker, at an internal (non-terminal) position in one of the at least two effector molecules covalently linked by the linker, or at a position in the linker.

In some embodiments, the multi-targeted molecule comprising at least two effector molecules covalently linked together is conjugated with at least one ligand. Without limitations, the ligands can be the same or they can be different. The two ligands can be conjugated independently at any position in the multi-targeted molecule. For example, a first ligand can be present in the first effector molecule and the second ligand can be present in the linker connecting the first effector molecule to a second effector molecule or a first ligand can be present in the first effector molecule and the second ligand can be present in the second effector molecule covalently that is covalently linked to the first effector molecule; or both ligands can be present in the same effector molecule; or both ligands can be present in the linker connecting the effector molecules.

In some embodiments, the linker connecting the effector molecules comprises a ligand. Without limitations, the ligand can be present at any position in the linker. For example, the ligand can be conjugated to the middle position or within 1, 2, or 3 monomers or units at middle of the linker.

In some embodiments, the multi-targeted molecule is assembled from two siRNAs, wherein the two siRNAs are linked to each other covalently via a nucleotide-based or non-nucleotide based linker. In some embodiments, the linker connecting the two siRNAs comprises the nucleotide sequence uuu or (dT)n, where n is 1-20. In some embodiments, the linker connecting the effector molecules comprises a molecule selected from the group consisting of:
—(CH₂)₁₂—(“C12 linker” or “Q50”),
—(CH₂)₆—S—S—(CH₂)₆—(“C6-S—S—C6 linker” or “Q51”),
Q151,
Q173,
—CH₂CH₂O—(CH₂CH₂)ₙ—CH₂CH₂O—CH₂CH₂O—, where n is 0 or 1-20;
—(CH₂)₉—(CH₂)ₙ—CH₂— where n is 0 or 1-20;
mono-, di-, tri-, tetra-, penta- or polyprolinol, optionally conjugated with a ligand; and
mono-, di-, tri-, tetra-, penta- or polyhydroxyprolinol, (e.g., poly[4-hydroxyprolinol]) optionally conjugated with a ligand.

In some embodiments, the multi-targeted molecule is assembled from two siRNAs wherein sense strand of the first siRNA is covalently linked to the sense strand of the second siRNA. Without limitations, the two sense strands can be linked to each other in any orientation. For example, 3-end of the first sense strand can be linked to 5′-end of the second sense strand; 3-end of the first sense strand can be linked to 3-end of the second sense strand; or 5′-end of the first sense strand can be linked to 5′-end of the second sense strand.

In some embodiments, the multi-targeted molecule is assembled from two siRNAs wherein antisense strand of the first siRNA is covalently linked to the antisense strand of the second siRNA. Without limitations, the two antisense strands can be linked to each other in any orientation. For example, 3-end of the first antisense strand can be linked to 5′-end of the second antisense strand; 3-end of the first antisense strand can be linked to 3-end of the second antisense strand; or 5′-end of the first antisense strand can be linked to 5′-end of the second antisense strand.

In some embodiments, the multi-targeted molecule is assembled from two siRNAs wherein sense strand of the first siRNA is covalently linked to the antisense strand of the second siRNA. Without limitations, the sense strand of the first siRNA can be linked to the antisense strand of the second siRNA in any orientation. For example, 3-end of the sense strand can be linked to 5′-end of the antisense strand; 3-end of the sense strand can be linked to 3-end of the antisense strand; or 5′-end of the sense strand can be linked to 5′-end of the antisense strand.

In some embodiments, the multi-targeted molecule is assembled from two siRNAs wherein sense strand of the first siRNA is covalently linked to the sense strand of the second siRNA and antisense strand of the first siRNA is covalently linked to the antisense strand of the second siRNA. In some embodiments, the multi-targeted molecule is assembled from two siRNAs wherein antisense strand of the first siRNA is covalently linked to the sense strand of the second siRNA and sense strand of the first siRNA is covalently linked to the antisense strand of the second siRNA.

In some embodiments, the linker is —[(P-Q″-R)_{q}—X—(P′-Q′″-R′)_{q}′]_{q}″-T-, wherein: P, R, T, P′, R′ and T are each independently for each occurrence absent, CO, NH, O, S, OC(O), NHC(O), CH₂, CH₂NH, CH₂O; NHCH(R^{a})C(O), —C(O)—CH(R^{a})—NH—, CH═N—O

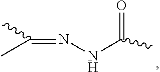

or heterocyclyl;

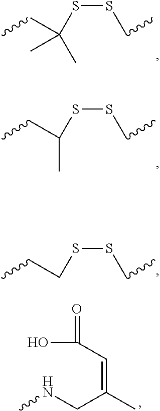

Q″ and Q′″ are each independently for each occurrence absent, —(CH₂)ₙ—, —C(R¹)(R²)(CH₂)ₙ—, —(CH₂)ₙC(R¹)(R²)—, —(CH₂CH₂O)ₘCH₂CH₂—, or —(CH₂CH₂O)ₘCH₂CH₂NH—;
X is absent or a cleavable linking group;
R^{a} is H or an amino acid side chain;
R¹ and R² are each independently for each occurrence H, CH₃, OH, SH or N(R^{N})₂;
R^{N} is independently for each occurrence H, methyl, ethyl, propyl, isopropyl, butyl or benzyl;
q, q′ and q″ are each independently for each occurrence 0-20 and wherein the repeating unit can be the same or different;
n is independently for each occurrence 1-20; and
m is independently for each occurrence 0-50.

In some embodiments, the linker comprises at least one cleavable linking group.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

A cleavable linking group is one which is sufficiently stable outside the cell, but which upon entry into a target cell is cleaved to release the two parts the linker is holding together. In a particular embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, an iRNA agent that targets cells in the CNS can be conjugated to a tether that includes a sialic acid (SA). CNS cells are enriched for neuramidase enzymes (e.g., neuramidase 1 (NEU1), neuramidase 2 (NEU2), neuramidase 3 (NEU3), neuramidase 4 (NEU4), and the like). In particular, NEU3 is enriched in the cells of the CNS and is localized to the inner membrane of the nuclear envelope while NEU1 is localized to the outer membrane of the nuclear envelope, as well as the plasma membrane (see e.g., Ledeen et al. (2011) New findings on nuclear gangliosides: overview on metabolism and function. 116(5):714-720). NEU3 cleaves terminal 2,3- and 2,6-linked SA (see e.g., U.S. Pat. No. 10,907,176).

In some embodiments, a cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

Additional exemplary cleavable linking groups include all those exemplary endosomal cleavable linkers as well as phosphoramidites, described herein below.

### Cleavable Linkers

In certain aspects provided herein are cleavable linkers, e.g., endosomal cleavable and/or protease cleavable. In some embodiments, a cleavable linker described herein can be comprised in a larger linker. In some embodiments, the cleavable linker is a carbohydrate linker that is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linker is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions). In some embodiments, the linker is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linkers as described herein and as known in the art can be used for any molecule for which cleavage in endo-lysosomal compartments would be useful. The cleavable linkers described herein and as known in the art can be particularly effective in pro-drug approaches especially for hydrophobic conjugates, attaching endosomal cleavable agents, or any other agents that may need to be activated or liberated in endo-lysosomal compartments.

Exemplary specific linkers of the effector molecules of the multi-targeted molecules, include, without limitation, all those endosomal cleavable linkers as well as phosphoramidites disclosed herein below.

### Effector Molecules

The skilled person is well aware that double-stranded oligonucleotides comprising a duplex structure of between 20 and 23, but specifically 21, base pairs have been hailed as particularly effective in inducing RNA interference (Elbashir et al., EMBO 2001, 20:6877-6888). However, others have found that shorter or longer double-stranded oligonucleotides can be effective as well.

As used herein, the term “siRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. As used herein, the terms “siRNA activity” and “RNAi activity” refer to gene silencing by an siRNA.

The double-stranded oligonucleotides comprise two oligonucleotide strands that are sufficiently complementary to hybridize to form a duplex structure. Generally, the duplex structure is between 15 and 35, more generally between 18 and 25, yet more generally between 19 and 24, and most generally between 19 and 21 base pairs in length. In some embodiments, longer double-stranded oligonucleotides of between 25 and 30 base pairs in length are preferred. In some embodiments, shorter double-stranded oligonucleotides of between 10 and 15 base pairs in length are preferred. In another embodiment, the double-stranded oligonucleotide is at least 21 nucleotides long.

In some embodiments, the double-stranded oligonucleotide comprises a sense strand and an antisense strand, wherein the antisense RNA strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

In some embodiments, the double-stranded region of a double-stranded oligonucleotide is equal to or at least, 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40 or more nucleotide pairs in length.

In some embodiments, the antisense strand of a double-stranded oligonucleotide is equal to or at least 14, 15, 16, 17, 18, 19, 20, 21, 22, 23, 23, 24, 25, 26, 27, 28, 29, 30, 31, 32, 33, 34, 35, 36, 37, 38, 39, or 40 or more nucleotides in length.

In some embodiments, the sense strand of a double-stranded oligonucleotide is equal to or at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, 40 or more nucleotides in length.

In some embodiments, one strand has at least one stretch of 1-10 single-stranded nucleotides in the double-stranded region. By “stretch of single-stranded nucleotides in the double-stranded region” is meant that there is present at least one nucleotide in the double-stranded region that is not basepaired with another nucleotide. When the stretch of single-stranded nucleotides is present internally (non-terminally) in the double-stranded region, at least one nucleotide base pair can be present at both ends of the single-stranded stretch. When present at the end of a double-stranded region, the stretch of single-stranded nucleotides can be a single-stranded overhang. The stretch of single-stranded nucleotides in the double-stranded region can be in the form of a bulge or one- or more mismatched nucleotides. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no non-basepaired nucleotides opposite to the single-stranded oligonucleotides of the first strand and vice versa (e.g., a single-stranded loop). In some embodiments, the single-stranded nucleotides are present within 8 nucleotides from either end, for example, 8, 7, 6, 5, 4, 3, or 2 nucleotide from either the 5′ or 3′ end of the region of complementarity between the two strands.

Hairpin and dumbbell type oligonucleotides will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length. In some embodiments, the nucleic acid based effector molecule is a hairpin oligonucleotides that can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligonucleotides that can induce RNA interference are also referred to as “shRNA” herein.

In certain embodiments, two oligonucleotide strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA and an RNA:RNA duplex.

Circular sciRNAs Structure Design

In this disclosure, the inventors have also designed a novel strategy to prepare a small circular interfering RNAs (sciRNAs) using chemically modified nucleotides and connecting the extremities of the nucleic acids of a sense strand, generating a circular sense construct with blocked 5′ and 3′ ends. For instance, exemplary sciRNAs have been synthesized with the antisense strand annealed to a 5′-3′ cyclized sense strand carrying a trivalent GalNAc ligand, prepared using “click” chemistry, and potent gene expression silencing in vitro and in vivo have been observed with these sciRNAs, especially the ones with phosphate mimic modifications at the 5′-end of an antisense nucleotide sequence, including, for instance, 5′-phosphorothioate (5′-PS), 5′-phosphorodithioate (5′-PS₂), 5′-vinylphosphonate (5′-VP), 5′-methylphosphonate (5′-MePhos), and 5′-deoxy-5′-C-malonyl modifications.

Also have been synthesized are exemplary bis-sciRNAs having two sense nucleotide sequences connected together by a bis-linker (e.g., a nucleotide-based or non-nucleotide-based cleavable linker), with the 5′ end of one sense nucleotide sequence cyclized with 3′ end of the other sense nucleotide sequence using “click” chemistry, forming a cyclized sense strand with bis-sense nucleotide sequences. One or two of the sense nucleotide sequences carry a lipophilic moiety (and/or a trivalent GalNAc ligand) at a non-terminal position of the sense nucleotide sequences. One or two antisense strand nucleotide sequences are annealed to the corresponding sense nucleotide sequence of the 5′-3′ cyclized sense strand.

Accordingly, one aspect of the invention relates to a small circular interfering RNA (sciRNA) comprising a sense strand and an antisense strand. Each of the sense and antisense strands comprises at least one nucleic acid modification.

In some embodiments, the sense strand has a circular or substantially circular structure. In some embodiments, the antisense strand has a circular or substantially circular structure.

The sense strand or antisense strand can form circular or substantially circular structure via a cycling linking moiety that connects one end of the sense (or antisense) strand to the other end of the sense (or antisense) strand. The circular or substantially circular structure of the sense or antisense strand may be formed by a cyclization procedure illustrated in Scheme 1. As shown in Scheme 1, a reactive linking moiety Q is added to one end of the sense (or antisense) strand and another reactive linking moiety Y is added to the other end of the sense (or antisense) strand. Q and Y each may contain various linkers (tethers) and carrier(s) which may carry ligand(s), and each contain a terminal functional group that are reactive to each other. Activating the reaction between Q and Y via an addition reaction would then form Z, a cycling linking moiety, which closes the cycle, forming a circular or substantially circular structure. An exemplary cyclization procedure via a click chemistry (e.g., forming a triazole from the azide-alkyne cycloaddition) is illustrated in Scheme I of Example 12.

Depending on the reactions used for the cyclization of the sense (or antisense) strand, and the linkers/cyclic groups contained in the reactive linking moieties Q and Y, the cycling linking moiety Z in the circular sense (or antisense) strand may contain one or more linkages selected from the group consisting of a triazole linkage, an amide linkage, a sulfide or disulfide linkage, a phosphate linkage, an oxime linkage, a hydrazo linkage, a N,N′-dialkylenehydrazo linkage, a methyleneimino linkage, a methylenecarbonylamino linkage, a methylenemethylimino linkage, a methylenehydrazo linkage, a methylenedimethylhydrazo linkage, a methyleneoxymethylimino linkage, a hydroxylamino linkage, a formacetal linkage, an alkyl or aryl linkage, a PEG linkage, an ether linkage, a thioether linkage, a thiodiester linkage, a thionocarbamate linkage, a thioacetamido linkage, a sulfonate linkage, a sulfonamide linkage, a sulfonate ester linkage, a thioformacetal linkage, an urea linkage, a carbonate linkage, an amine linkage, a maleimide-thioether linkage, a phosphodiester linkage, a phosphotriester linkage, a hydrogen phosphonate linkage, an alkyl or aryl phosphonate linkage, a phosphoramidate linkage, a phosphorothioate linkage, a phosphoroselenate linkage, a borano phosphate linkage, a borano phosphate ester linkage, a sulfonamide linkage, a carbamate linkage, a carboxamide linkage, a carboxymethyl linkage, a carboxylate ester linkage, a siloxane linkage, a dialkylsiloxane linkage, an ethylene oxide linkage, and combinations thereof.

In one embodiment, the cycling linking moiety Z in the circular sense (or antisense) strand may contain one or more linkages selected from the group consisting of a triazole linkage, an amide linkage, a disulfide linkage, a phosphate linkage, an oxime linkage, an alkyl linkage, a PEG linkage, an ether linkage, a thioether linkage, an urea linkage, a carbonate linkage, an amine linkage, a maleimide-thioether linkage, a phosphodiester linkage, a sulfonamide linkage, a carbamate linkage, and combinations thereof.

In certain embodiments, the cycling linking moiety may further contain one or more carriers that may serve to connect a ligand to the sciRNA. The carrier may be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuranyl, and decalinyl. In one embodiment, the acyclic group is a moiety based on a serinol backbone or a diethanolamine backbone.

One exemplary cycling linking moiety contains a triazole linkage formed through a cyclization procedure via a click chemistry (e.g., from the azide-alkyne cycloaddition). As discussed above in Scheme 1, the cyclization can be formed by attaching a reactive linking moiety Q to one end of the sense (or antisense) strand, attaching another reactive linking moiety Y to the other end of the sense (or antisense) strand, and activating the reaction between Q and Y. The Q/Y pair in this case is azide/alkyne pair. Non-limiting exemplary molecules that contain the reactive linking moiety Q/Y (in this case, azide/alkyne functional groups) are illustrated below.

| | |
|---|---|
| L123 N-(hexynylcarbox- amidocaproyl)-4- hydroxyprolinol (Hyp-hexynyl) | 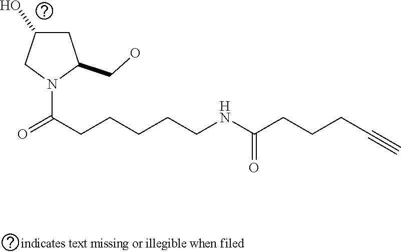 [SMILES: C#CCCCC(=O)NCCCCCC(=O)N1C[C@H](O)C[C@H]1CO] |
| L146 N-(benzocyclooctynyl- carboxamidoca- proylamidocaproyl)-4- hydroxyprolinol (Hyp-C6-C6-octynyl) | 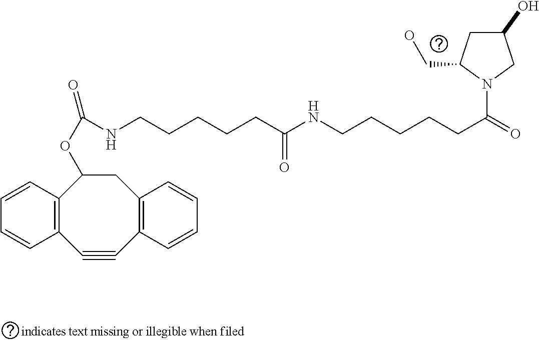 [SMILES: O=C(CCCCCNC(=O)OC1Cc2ccccc2C#Cc2ccccc21)NCCCCCC(=O)N1C[C@H](O)C[C@H]1CO] |
| L177 N-(azido-PEG4- carboxamidocaproyl)- 4-hydroxyprolinol (Hyp-C6-PEG4-N3) | 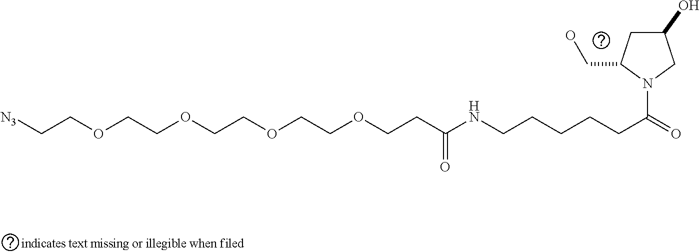 [SMILES: [N-]=[N+]=NCCOCCOCCOCCOCCC(=O)NCCCCCC(=O)N1C[C@H](O)C[C@H]1CO] |
| L347 N-(propargyl ether carboxamidocaproyl)-4- hydroxyprolinol | 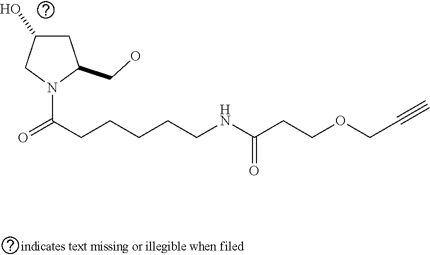 [SMILES: C#CCOCCC(=O)NCCCCCC(=O)N1C[C@H](O)C[C@H]1CO] |
| Q44 N-(hexinylcarbox- amidocaproyl)- prolinol-4-phosphate (Hyp-hexinyl) | 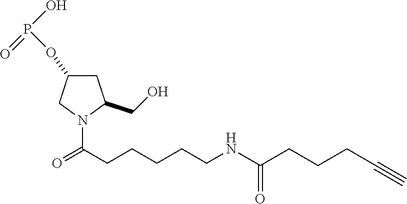 |
| Q84 N-(hexynyl)-prolinol- 4-phosphate (Hyp-hexynyl) L124 N-(hexynyl)-4- hydroxyprolinol | 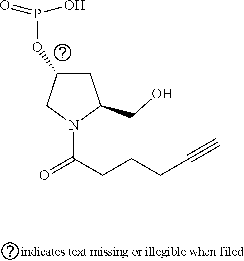 |
| Q99 N-(benzocyclooctynyl- carboxamidoca- proylamidocaproyl)- prolinol-4-phosphate (Hyp-C6-C6- octynyl) | 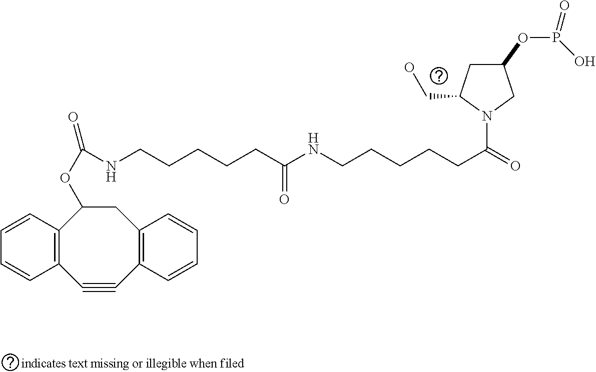 |
| Q127 N-(azido-PEG4- carboxamidocaproyl)- prolinol-4-phosphate (Hyp-C6-PEG4- N3) | 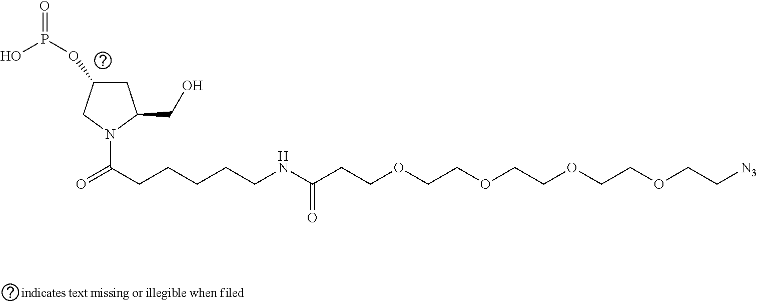 |
| Q136 N-(6- Azidohexylcarbox- amidocaproyl)- prolinol-4-phosphate (Hyp-C6-C6- Azido) | 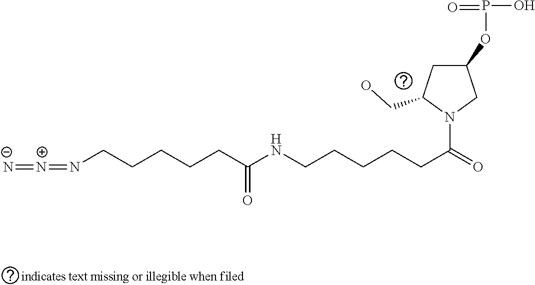 |
| Q325 Q8 with C6-DBCO | 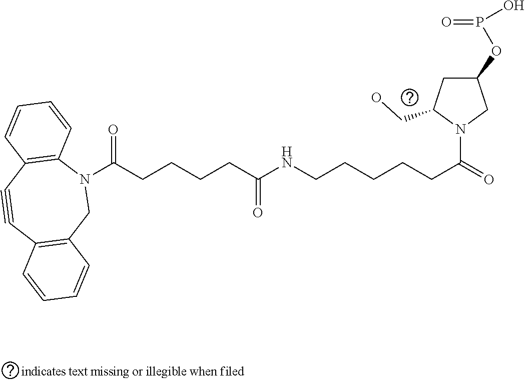 |
| Q186 5-hexyn-lyl-phosphate | 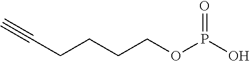 |
| Q187 10-(6-oxo-6-(dibenzo [b,f]azacyclooct- 4-yn-1-yl)-capramido- N-ethyl)-O- triethyleneglycol- 1-phosphate (DBCO-TEG- (10-1941)) | 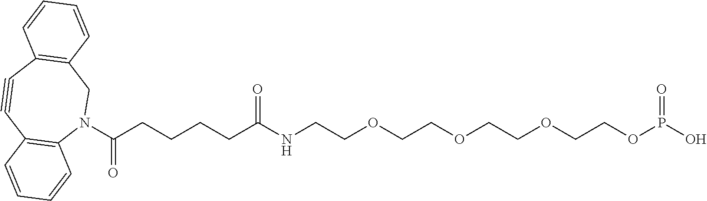 |
| Q301 6-azidohexyl phosphate | 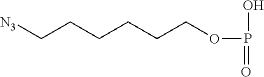 |
| Q301S 6-azidohexyl phosphorothioate | 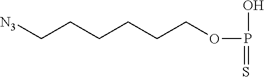 |
| Q396 N-(aminocaproyl- propargyl ether)prolinol- 4-phosphate | 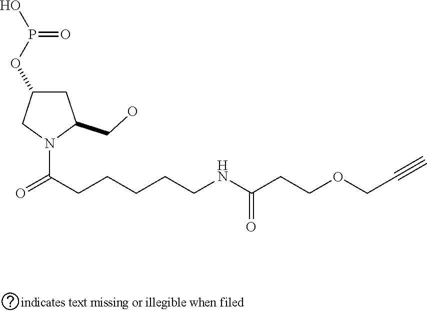 |
| Q397 N-(aminocaproyl- propargyl ether)-(S)- pyrrolidin-3-ol- phosphate | 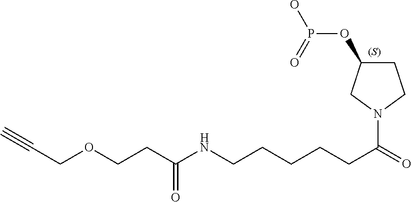 |
| Q399 2,3,5,6-tetrafluoro- 4-azido-benzoyl-N- aminocaproyl-(S)- pyrrolidin-3-ol- phosphate | 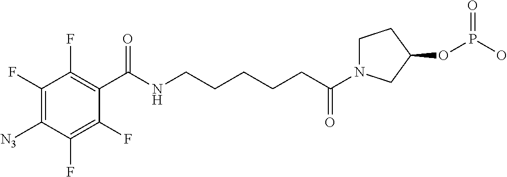 |
| Q422 N-(hexynylcarbox- amidocaproyl)-(S)- pyrrolidin-3-ol- phosphate | 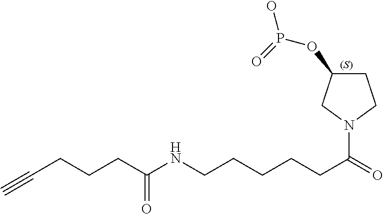 |
| Apy 2′-O-propynyl- adenosine-3′-phosphate | 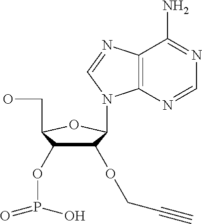 |
| Cpy 2′-O-propynyl- cytidine-3′-phosphate | 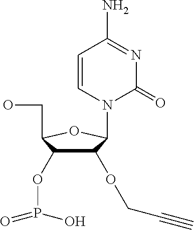 |
| Upy 2′-O-propynyl- uridine-3′-phosphate | 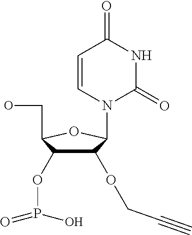 |
| Tpy 2′-O-propynyl-5- methyl-uridine-3′- phosphate | 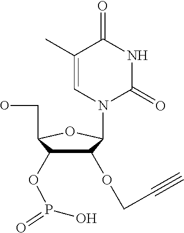 |
| Gpy 2′-O-propynyl- guanosine-3′-phosphate | 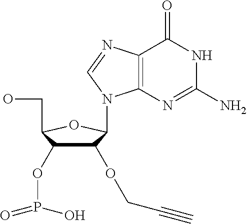 |

As discussed above, these exemplary molecules may be attached to the end of an oligonucleotide strand via, e.g., a phosphate. Activating the click chemistry between the reactive linking moieties between the Q/Y pair would form a cyclized oligonucleotide strand. For instance, attaching L123 and Q301 (illustrated in the above table) to each end of an oligonucleotide strand via a phosphate and clicking the azide/alkyne pair in L123 and Q301 would form

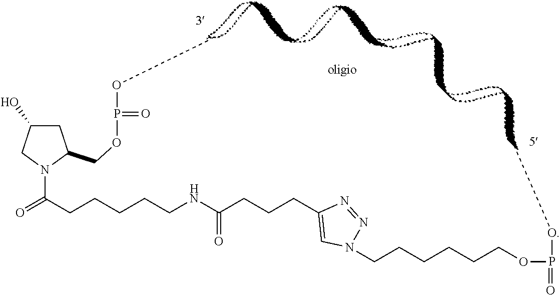

(Z49-cyclization by clicking 3′-phosphate-Hyp-C9-1,4-triazole-C6-5′-phosphate).

Additional non-limiting examples of the cycling linking moieties Z formed by clicking the above-illustrative reactive linking moiety Q/Y pairs are illustrated below.

| | | |
|---|---|---|
| Q310 | Click Q187 and Q127 | 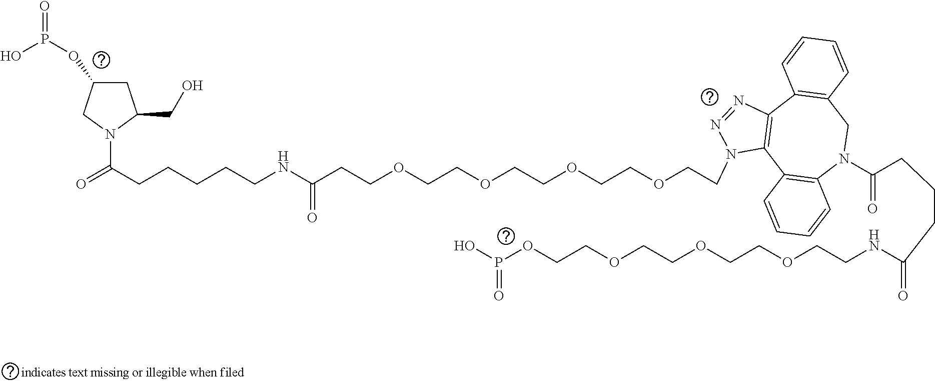 |
| Q324 (DBCO- azide 5′-3′ link) | Click Q187 and L177 | 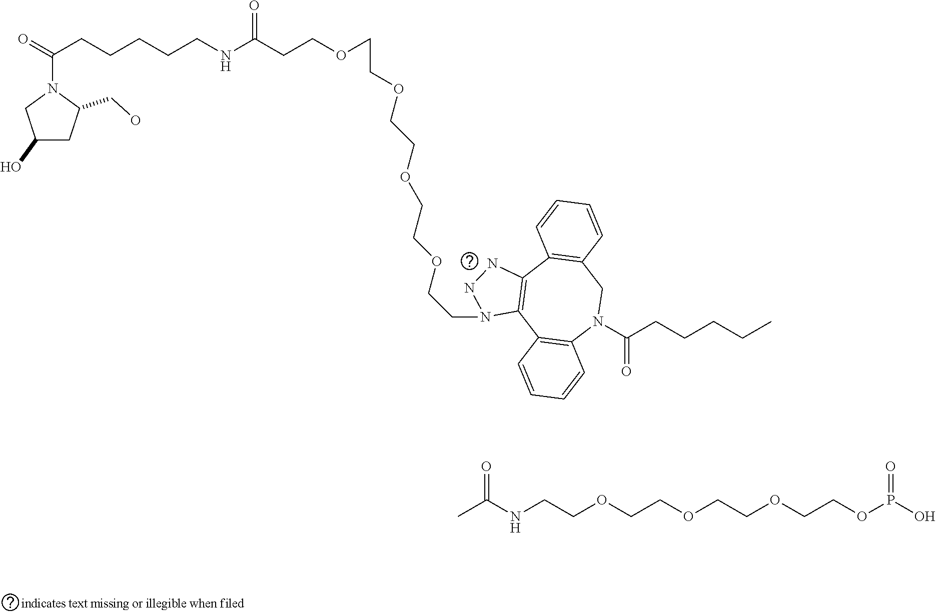 |
| Q327 (DBCO- azide 5′-3′ link (Q8 based)) | Click Q325 and L177 | 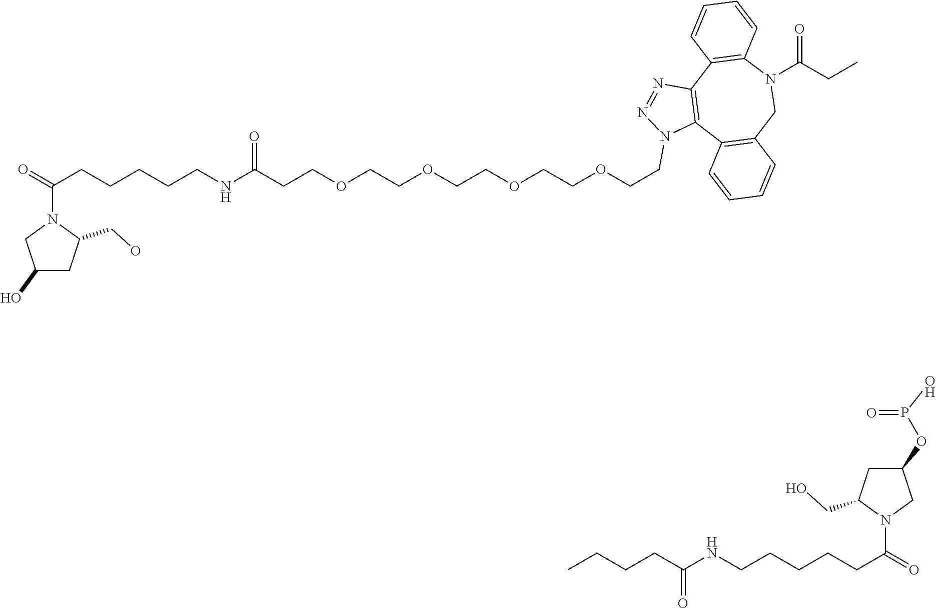 |
| Q328 (DBCO- azide 5′-5′ link (Q8 based)) | Click Q325 and Q301 | 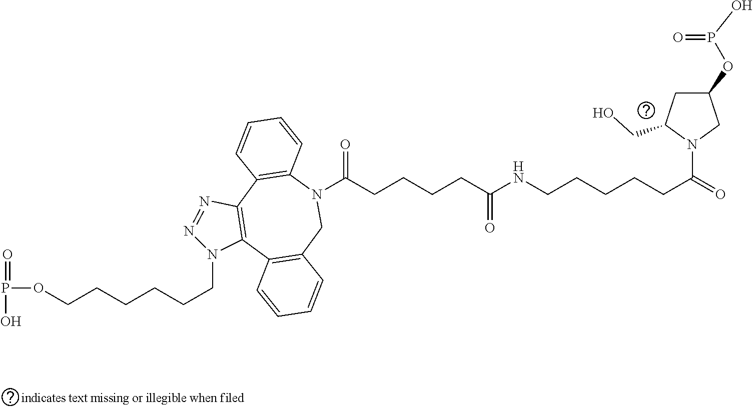 |
| Q138 Q8-C6- diphenyl- cycloocta (1,2,3 triazole)- C6-C6-Hyp | Click Q99 and Q136 | 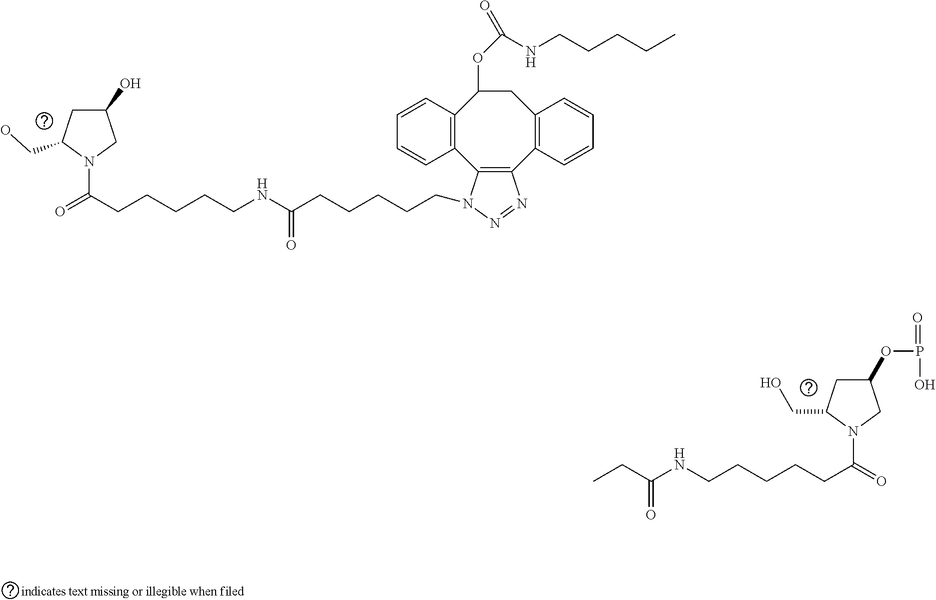 |

One exemplary cycling linking moiety contains a maleimide-thioether linkage (or thiosuccinimide linkage) formed through a cyclization procedure via a click chemistry from the thiol-maleimide addition reaction. As discussed above in Scheme 1, the cyclization can be formed by attaching a reactive linking moiety Q to one end of the sense (or antisense) strand, attaching another reactive linking moiety Y to the other end of the sense (or antisense) strand, and activating the reaction between Q and Y. The Q/Y pair in this case is thiol/maleimide pair. Non-limiting exemplary molecules that contain the reactive linking moiety Q/Y (in this case, thiol/maleimide functional groups) are illustrated below.

| | |
|---|---|
| Q157 N-[4-(N-maleimidomethyl)cyclohexane- 1-carboxamidocaproyl)prolinol-4- phosphate (Hyp-C6-maleimide) | 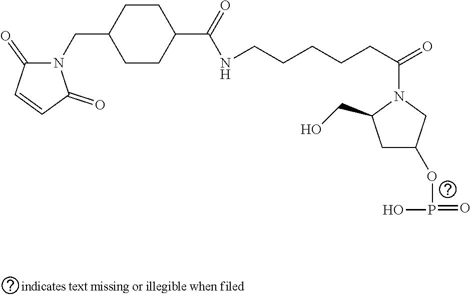 |
| Q385 N-[4-(N-maleimidomethyl)cyclohexane- 1-carboxamidocaproyl)-(S)-pyrrolidin-3- ol-phosphate (Q358-maleimide) | 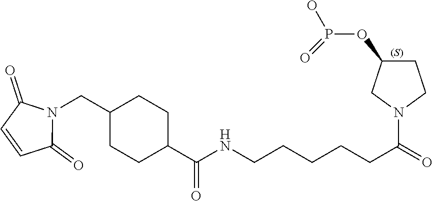 |
| Y224 2′ maleimide adenosine-3′ phosphate (attached via (Aah)) | 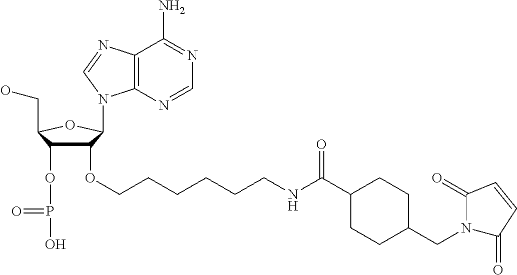 |
| Q5 6-(2-mercapto-acetylamino)-hexyl phosphate | 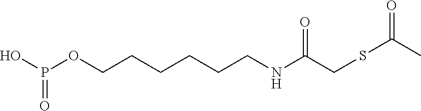 |
| Q51 6-hydroxyhexyldithiohexylphosphate (Thiol-Modifier C6 S-S Glen Res. 10- 1936) | 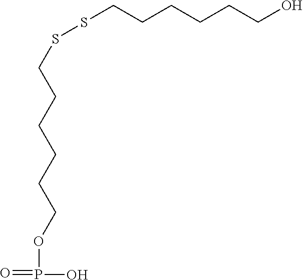 |
| Q66 6-mercaptohexylphosphate (Glen Res. 10-1936) | 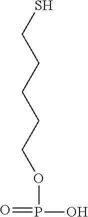 |
| Q82 1-thiohexylphosphate (after cleavage of Thiol-Modifier C6 S-S Glen Res. 10- 1936) | 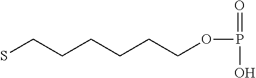 |
| Q332 6-(3-mercaptopropanamido)hexyl phosphate(5′-aminomodifier with thiol for conjugation) | 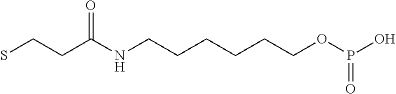 |
| (dTbm) S-isobutyryl-(2-mercaptoethylglycol)-2′- deoxythymidine-3′-phosphotriester (dT- BMEG) | 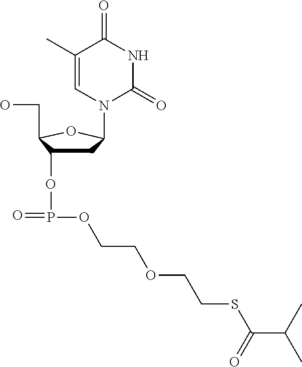 |
| (ubm) S-isobutyryl-(2-mercaptoethylglycol)- 2′O-methyluridine-3′-phosphotriester (u- BMEG) | 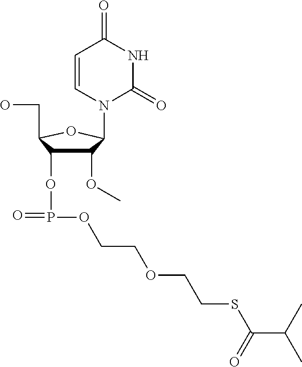 |
| (Ufbm) S-isobutyryl-(2-mercaptoethylglycol)-2′- fluorouridine-3′-phosphotriester (fU- BMEG) | 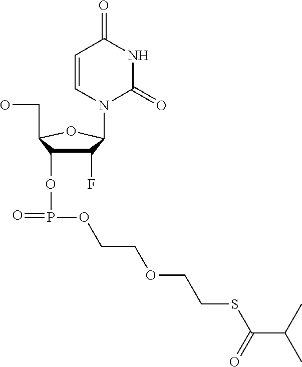 |
| Y138 2′-O-[{2-(butyldisulfaneyl)-2- methylpropyl}carbamate]-uridine-3′- phosphate | 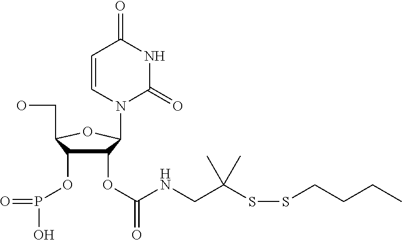 |

The sense strand can be at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length. In one embodiment, the sense strand is at least 20 nucleotides in length. In one embodiment, the sense strand is at least 40 nucleotides in length.

The antisense strand can be at least 10, 11, 12, 13, 14, 15, 16, 17, 18, 19, 20, 21, 22, 23, 23, 24, 25, 26, 27, 28, 29, 30, 31, 32, 33, 34, 35, 36, 37, 38, 39, or 40 nucleotides in length.

The antisense strand is annealed with the sense strand to form at least a partial duplex region. In some embodiments, one or more sense nucleotide sequences are annealed with the antisense strand. In some embodiments, at least one sense nucleotide sequence is not annealed with the antisense strand.

In some embodiments, the sense nucleotide sequence not annealed with the antisense strand can be a single-stranded oligonucleotide, such as an antisense oligonucleotide (ASO), an antimiR (antagomir) oligonucleotide, or a single-stranded siRNA (ss-siRNA) oligonucleotide.

In some embodiments, a duplex region is formed between the sense strand and antisense strand at least at the seed region of the antisense strand (i.e., at positions 2-8 of the 5′-end of an antisense nucleotide sequence).

Increasing the length of the sense strand, therefore the length of the duplex region, can have an impact on melting temperature of the sciRNA and can increase the thermal stability of the sciRNA duplex.

Increasing the length of the sense strand can be achieved by using a single sense nucleotide sequence, or by having more than one sense nucleotide sequences in the sense strand.

In some embodiments, the sense strand can have a long circular sense nucleotide sequence, having at least 20 nucleotides in length, at least 25 nucleotides in length, or at least 30 nucleotides in length, for instance, having 20 to 45 nucleotides in length, or 30 to 45 nucleotides in length.

In some embodiments, the antisense strand comprises at least one antisense nucleotide sequence. The at least one antisense nucleotide sequence has about 20 to about 45 nucleotides in length.

In one embodiment, a long circular sense nucleotide sequence is annealed with an antisense strand having about 19 to about 23 nucleotides in length, complementary to a target mRNA transcript nucleotide sequence. In one embodiment, a long circular sense nucleotide sequence is annealed with two or more antisense nucleotide sequences having about 19 to about 23 nucleotides in length, complementary to two or more target mRNA transcript nucleotide sequences.

The long circular sense nucleotide sequence may be a substrate cleavable by DICER.

### Bis-sciRNA Compounds Structural Design

A further aspect of the invention relates to a small circular interfering RNA (sciRNA) for modulating one or more target mRNAs in the central nervous system (CNS) of a subject, comprising a first strand having at least 40 nucleotides in length and at least two first strand nucleotide sequences connected together by a bis-linker, each nucleotide sequence having about 18 to about 28 nucleotides in length, and at least one second strand nucleotide sequence, having about 19 to about 23 nucleotides in length, annealed with at least one of the first strand nucleotide sequences. The first strand has a circular or substantially circular structure. Each of the first strand nucleotide sequences and the second strand nucleotide sequence(s) comprises at least one nucleic acid modification. The first strand nucleotide sequences or the second strand nucleotide sequence(s) comprise one or more ligands.

The first strand in the bis-sciRNA comprises two or more nucleotide sequences connected together by a bis-linker, and can be referred to herein as the “bis-strand” (e.g., bis-sense strand or bis-antisense strand).

Additionally, the first strand in the bis-sciRNA has a circular or substantially circular structure, and can be referred to herein as the “circular or substantially circular strand.” The first strand comprises at least two first strand nucleotide sequences, and is formed by connecting the at least two first strand nucleotide sequences together with a bis-linker. Each of the at least two first strand nucleotide sequences can be annealed with a same or different second strand nucleotide sequences. Each of the first/second strand nucleotide sequence can target a same or different RNA molecule. Therefore, the bis-sciRNA molecules can target one or more target mRNA.

The bis-sciRNA molecules can be multi-targeted molecules. The multi-targeted molecules include at least two nucleic acid-based effector molecules that are linked to each other by a bis-linker moiety as described herein. By a “nucleic acid-based effector molecule” is meant a modified or unmodified nucleic acid molecule capable of modulating the activity of expression of a target nucleic acid (e.g., a target mRNA). It is noted that the at least two effector molecules are two separate effector molecules. In other words, the at least two effector molecules do not overlap with each other. Thus, provided herein are bis-sciRNA molecules designed to target one or more target nucleic acid, or two or more distinct target RNA sequences within one or more target nucleic acids, and that exhibit delivery to and surprising efficacy in a CNS tissue of a subject upon contact. Two target RNA sequences within a single target RNA are considered “distinct” when the target RNA sequences do not overlap with each other.

Thus, the multi-targeted molecules disclosed herein differ from molecules where one effector molecule is directed to two different targets, for example, double-stranded effector molecules where each strand is directed to a different target or an effector molecule comprising a sequence, wherein at least a portion of the sequence is complementary to or can hybridize with two different target sequences.

For instance, the circular or substantially circular sense strand may contain two sense nucleotide sequences, forming a bis-sciRNA. The circular or substantially circular sense strand can contain two symmetrical nucleotide sequences, or two asymmetrical nucleotide sequences. In the symmetrical scenario, each of the sense nucleotide sequence in the circular or substantially circular sense strand (e.g., each sense nucleotide sequence may have about 19 to about 23 nucleotides in length, e.g., 20-21 nucleotides in length) can be annealed with two identical antisense nucleotide sequences (e.g., each may have 21 nucleotides in length), targeting the same mRNA transcript nucleotide sequence. In the asymmetrical scenario, each of the sense nucleotide sequence in the circular or substantially circular sense strand (e.g., each sense nucleotide sequence may have about 19 to about 23 nucleotides in length, e.g., 20-21 nucleotides in length) can be annealed with two different antisense nucleotide sequences (e.g., each may have 23 nucleotides in length), targeting two different mRNA transcript nucleotide sequences.

Exemplary circular or substantially circular sense strands (or bis-sense strands) and circular sciRNA (or bis-sciRNA) are shown in Schemes 1A-1C. Schemes 1A and 1B each illustrate a circular or substantially circular sense strand containing two symmetrical (Scheme 1A) or asymmetrical (Scheme 1B) sense nucleotide sequences (with a total length of the bis-sense strand of 42-45 nucleotides). The two sense nucleotide sequences are connected by a bis-linked (e.g., nucleotide-based or non-nucleotide based linker (tether)). Scheme 1C illustrates a circular or substantially circular sense sense strand containing a long dicer-cleavable sense nucleotide sequence (e.g., 30 to 45 nucleotides), annealed with a shorter antisense nucleotide sequence (e.g., 19-23 nucleotides).

The circular or substantially circular structure of the sense strand or bis-sense strand may be formed by click chemistry by the same reaction mechanism as shown in Scheme 1 discussed above. In the case of the bis-sense strand, the circular or substantially circular structure of the bis-sense strand may be formed by clicking the 5′ end of one sense nucleotide sequence with the 3′ end of the other sense nucleotide sequence. The cycling linking moiety Z contains the combination of one or more of phosphate linkage, alkyl linkage, triazole linkage, amide linkage, and pyrrolidinyl cyclic group, with or without a ligand (L) carried by the cyclic group.

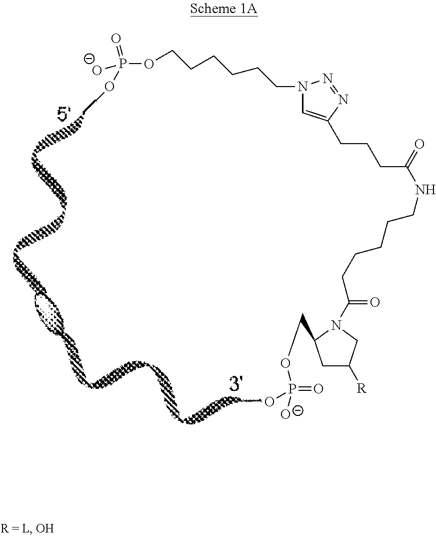

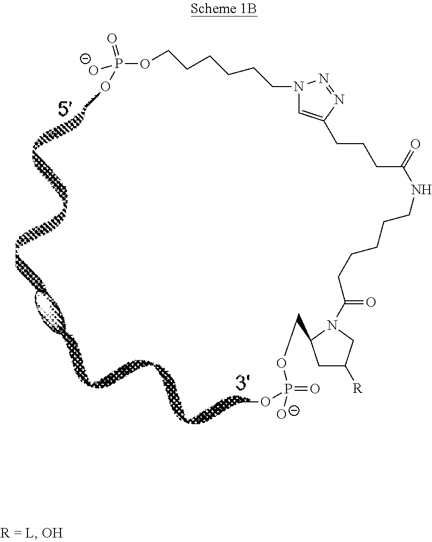

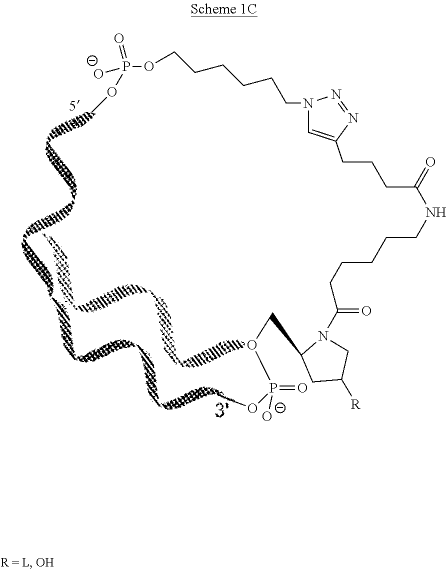

Additional exemplary circular or substantially circular sense strands (or bis-sense strands) and circular sciRNA (or bis-sciRNA) are shown in Schemes 2A-2C, Schemes 3A-3C, Schemes 4A-4C, Schemes 5A-5C, Schemes 6A-6C, Schemes 7A-7C, and Schemes 8A-8C, illustrating various cyclization reactions and cycling linking moieties. The sense nucleotide sequences and antisense strand nucleotide sequences in these schemes reflect those in Schemes 1A-1C. The cyclization reaction in these schemes are different than those in Schemes 1A-1C. For instance, in Schemes 2A-2C, the cyclization is by amide formation. The cycling linking moiety Z contains the combination of one or more of phosphate linkage, alkyl linkage, amide linkage, and a pyrrolidinyl cyclic group, with or without a ligand (L) carried by the cyclic group. In Schemes 3A-3C, the cyclization is by disulfide formation. The cycling linking moiety Z contains the combination of one or more of phosphate linkage, alkyl linkage, disulfide linkage, amide linkage, and pyrrolidinyl cyclic group, with or without a ligand (L) carried by the cyclic group. In Schemes 4A-4C, the cyclization is by click chemistry. The cycling linking moiety Z contains the combination of one or more of phosphate linkage, alkyl linkage, triazole linkage, amide linkage, and PEG linkage. In Schemes 5A-5C and 6A-6C, the cyclization is by oxime formation. The cycling linking moiety Z contains the combination of one or more of phosphate linkage, alkyl linkage, oxime linkage (aldoxime or ketoxime), amide linkage, and pyrrolidinyl cyclic group, with or without a ligand (L) carried by the cyclic group. In Schemes 7A-7C and 8A-8C, the cyclization is by hydrazone formation. The cycling linking moiety Z contains the combination of one or more of phosphate linkage, alkyl linkage, hydrazo linkage, amide linkage, and pyrrolidinyl cyclic group, with or without a ligand (L) carried by the cyclic group.

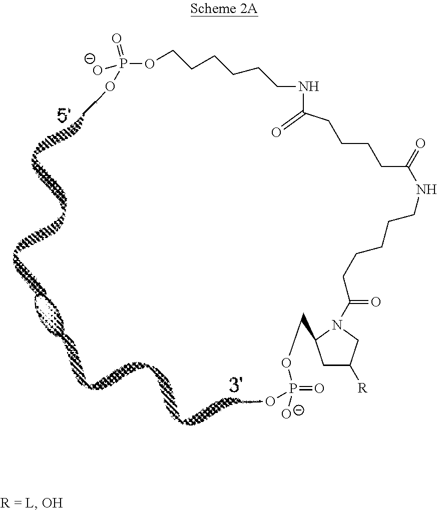

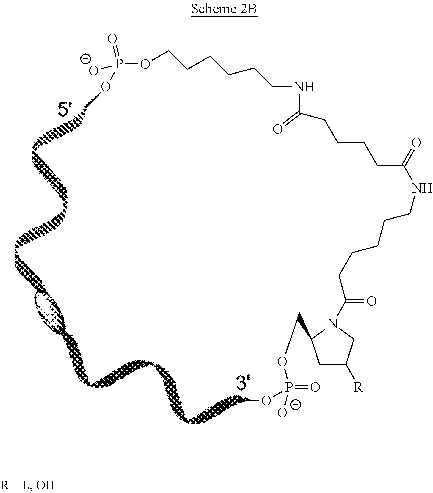

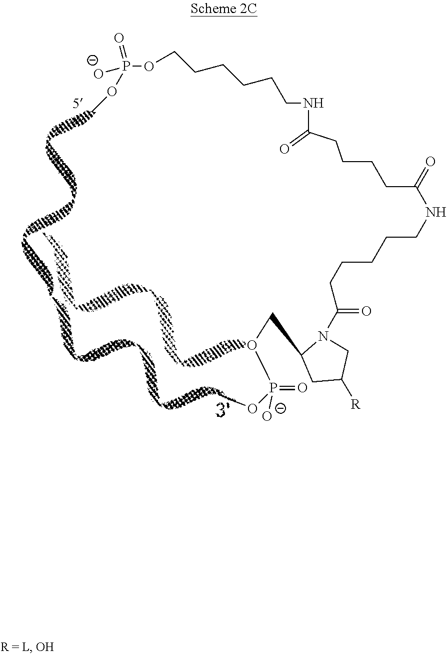

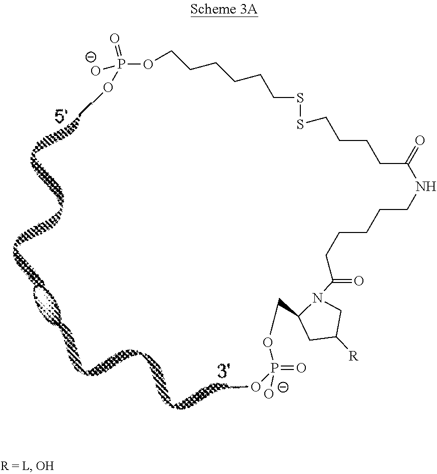

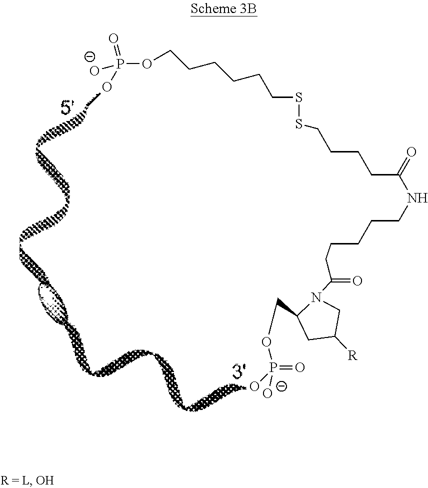

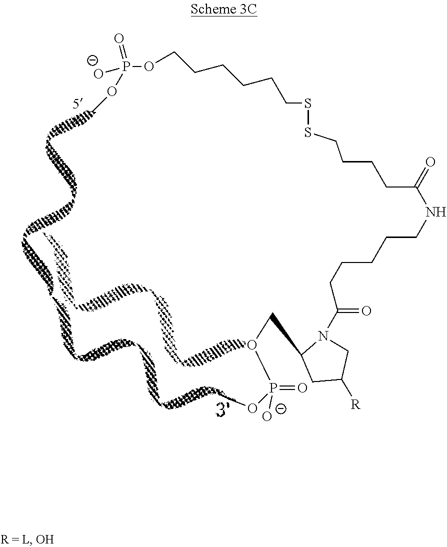

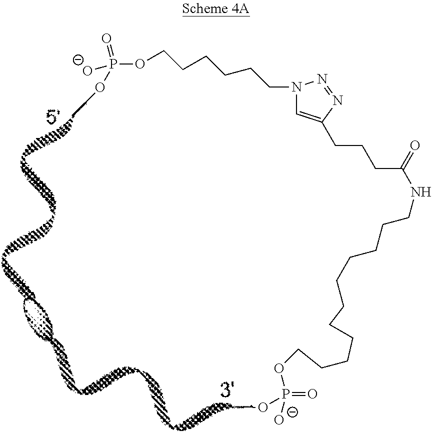 [SMILES: COP(=O)([O-])OCCCCCCCCCCCNC(=O)CCCc1cn(CCCCCCOP(=O)([O-])OC)nn1]

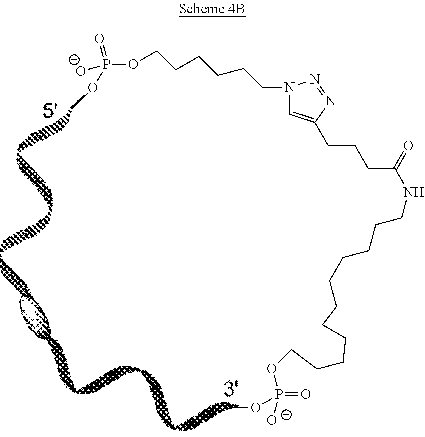 [SMILES: COP(=O)([O-])OCCCCCCCCCCCNC(=O)CCCc1cn(CCCCCCOP(=O)([O-])OC)nn1]

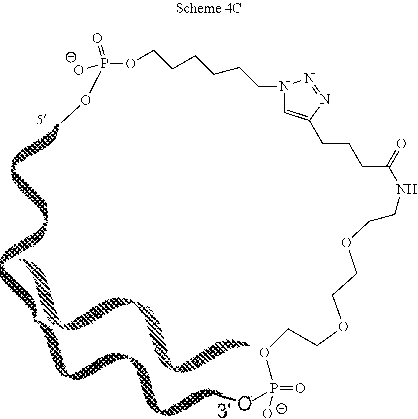 [SMILES: COP(=O)([O-])OCCCCCCn1cc(CCCC(=O)NCCOCCOCCOP(C)(=O)[O-])nn1]

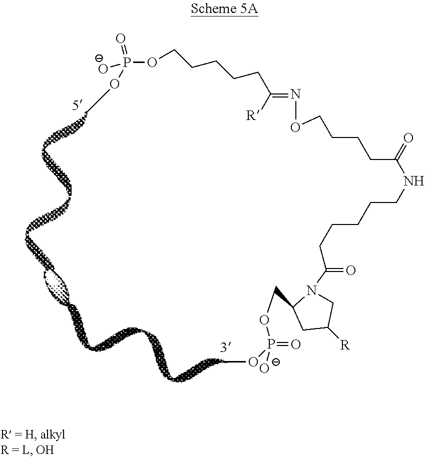

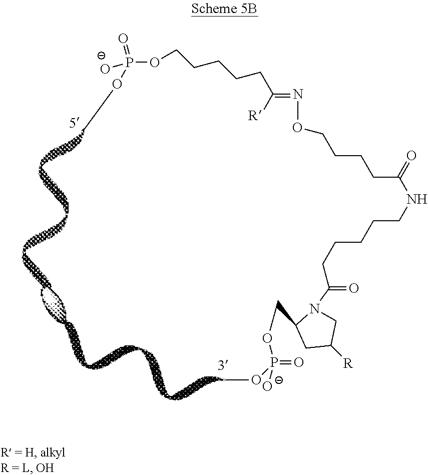

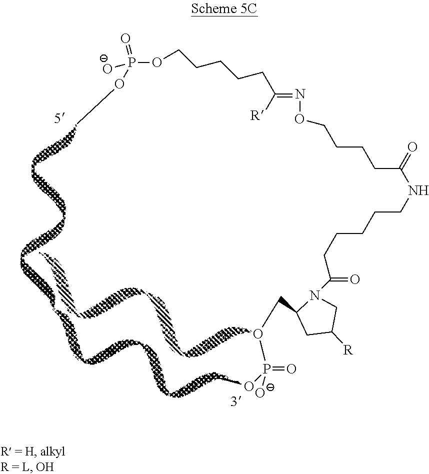

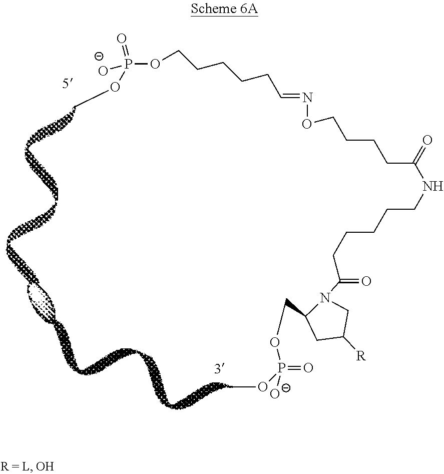

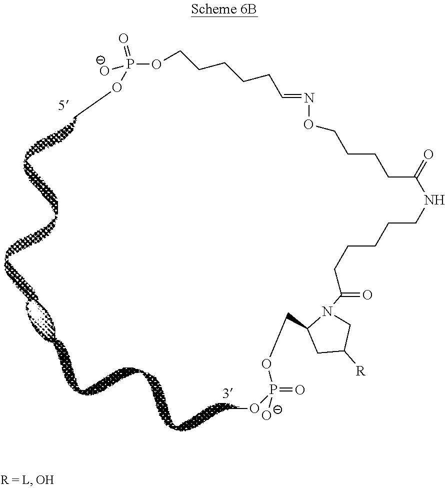

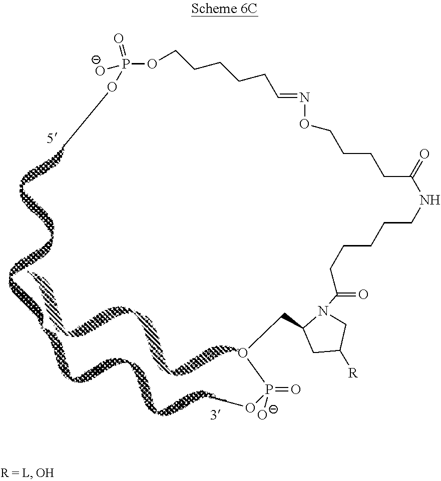

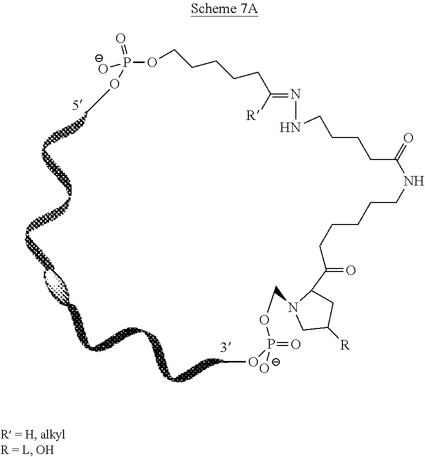

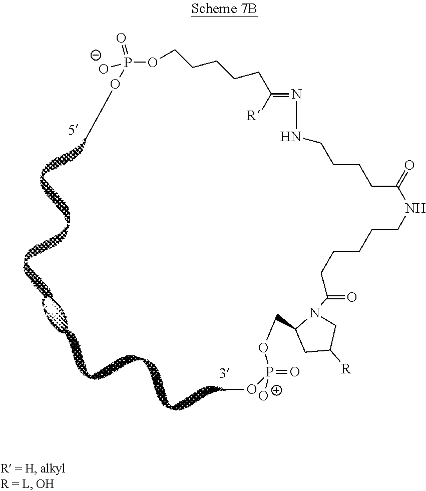

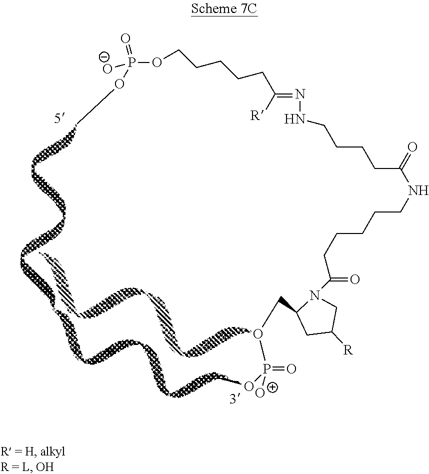

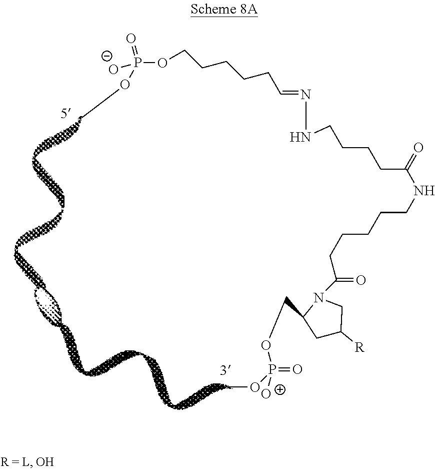

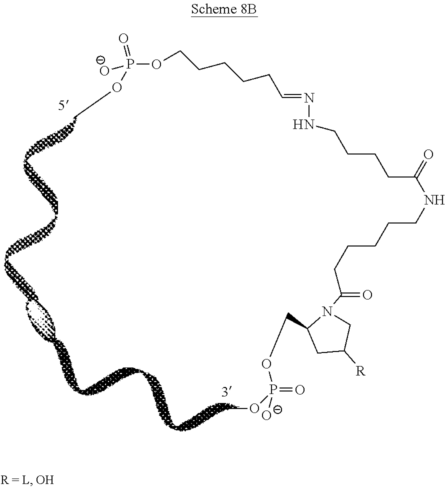

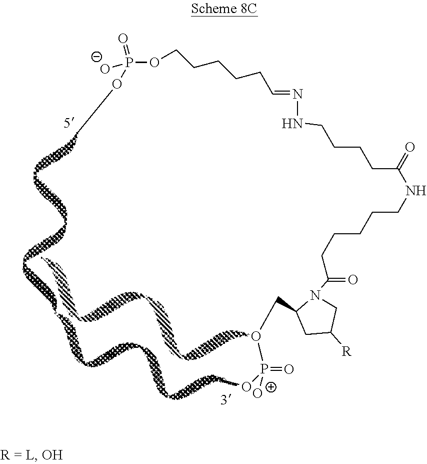

Additional exemplary bis-sciRNA design include those bis-sciRNAs illustrated in Example 13, in which a circular or substantially circular sense strand containing two asymmetrical sense nucleotide sequences (each sense nucleotide sequence has a length of 20-21 nucleotides). The two sense nucleotide sequences are connected by a bis-linked (e.g., 3 nucleotides in length). Each sense nucleotide sequence is annealed with a longer, different antisense nucleotide sequence (each has a length of 23 nucleotides). The cycling linking moiety Z contains the combination of one or more of phosphate linkage, alkyl linkage, and triazole linkage.

The examples shown in Schemes 1A-1C to Schemes 8A-8C and in Example 13 are for illustrative purpose only. The cyclization reactions and cycling linking moieties illustrated for cyclization of the sense nucleotide sequences of the sense strand (or bis-sense strand) would be applicable to the cyclization of the antisense nucleotide sequences of the antisense strand (or bis-antisense strand).

### Linkers/Tethers

Linkers/Tethers may be contained in the bis-sense strand or bis-antisense strand as part of the bis-linker to connect two sense nucleotide sequences (to form bis-sense strand) or antisense nucleotide sequences (to form bis-antisense strand) of the multi-targeted molecules (e.g., the effector molecules such as bis siRNA or the scriRNA (or bis-sciRNA)).

Linkers/Tethers may be contained as part of the cycling linking moiety of the circular or substantially circular sence strand (or circular or substantially circular bis-sense strand) of the sciRNA (or bis-sciRNA).

Linkers/tethers can also be used to connect the ligand to the multi-targeted molecules (e.g., the effector molecules such as bis siRNA or the scriRNA (or bis-sciRNA)), e.g., via a carrier.

The terms “linker,” “linkage,” “linking group,” “tether” can be used interchangeably.

Linkers in the sense strand (or bis-sense strand) or antisense strand (or bis-antisense strand) may be a nucleotide-based or non-nucleotide-based linker. The linker may be a stable linker that is stable in a biological fluid (e.g., in plasma or artificial cerebrospinal fluid). Alternatively, the linker may be a cleavable linker (e.g., a bio-cleavable linker).

Linkers/tethers may be connected to a ligand at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the ligand. Non-limited examples of linkers/tethers (underlined) include TAP-(CH₂)ₙNH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—; or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙONH(LIGAND); TAP-C(O)(CH₂)ₙONH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O)OC₆F₅; or TAP-NR″″(CH₂)ₙC(O)OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether.

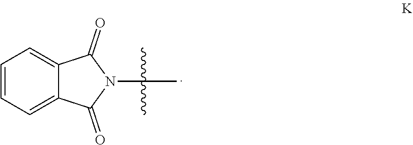 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

The cleavable linker may be cleavable in various tissue and cell structures, e.g., in liver homogenates, liver tritosomes, liver lysosomes, liver cytosol, brain homogenates, brain tritosomes, brain lysosomes, or brain cytosol.

### Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### Biocleavable Linkers/Tethers

The linkers can also include biocleavable linkers that are nucleotide and non-nucleotide linkers, or combinations thereof, that connect two parts of a molecule. For example, a biocleavable linker may connect one or both strands of two individual siRNA molecule, to generate a bis(siRNA). In some embodiments, mere electrostatic or stacking interaction between two individual siRNAs can represent a linker.

The non-nucleotide linkers include tethers or linkers derived from monosaccharides, disaccharides, oligosaccharides, and derivatives thereof, aliphatic, alicyclic, hetercyclic, and combinations thereof.

In some embodiments, at least one of the linkers (tethers) is a bio-clevable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include, without limitation, the following endosomal cleavable linkers as well as phosphoramidites:

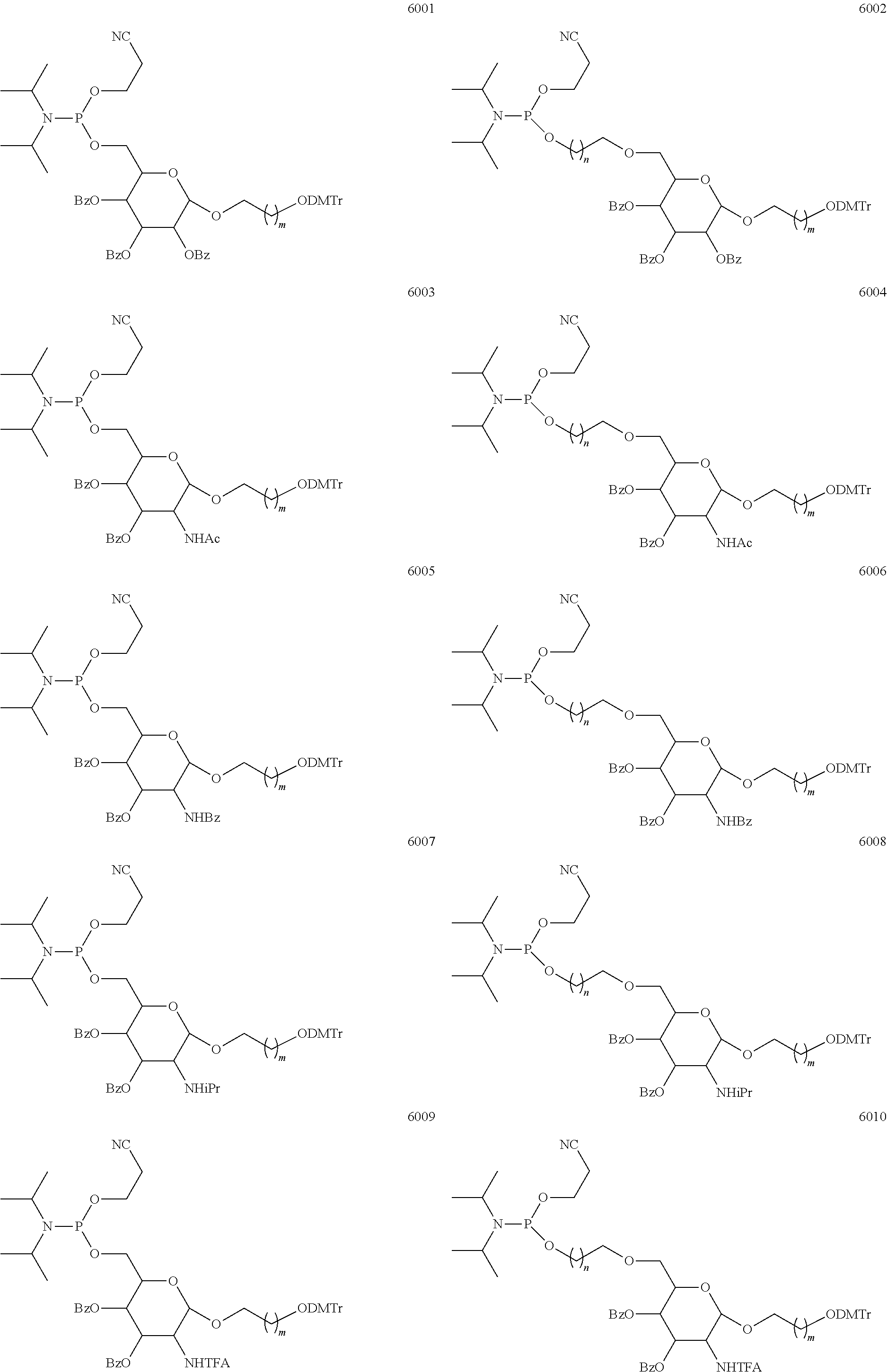

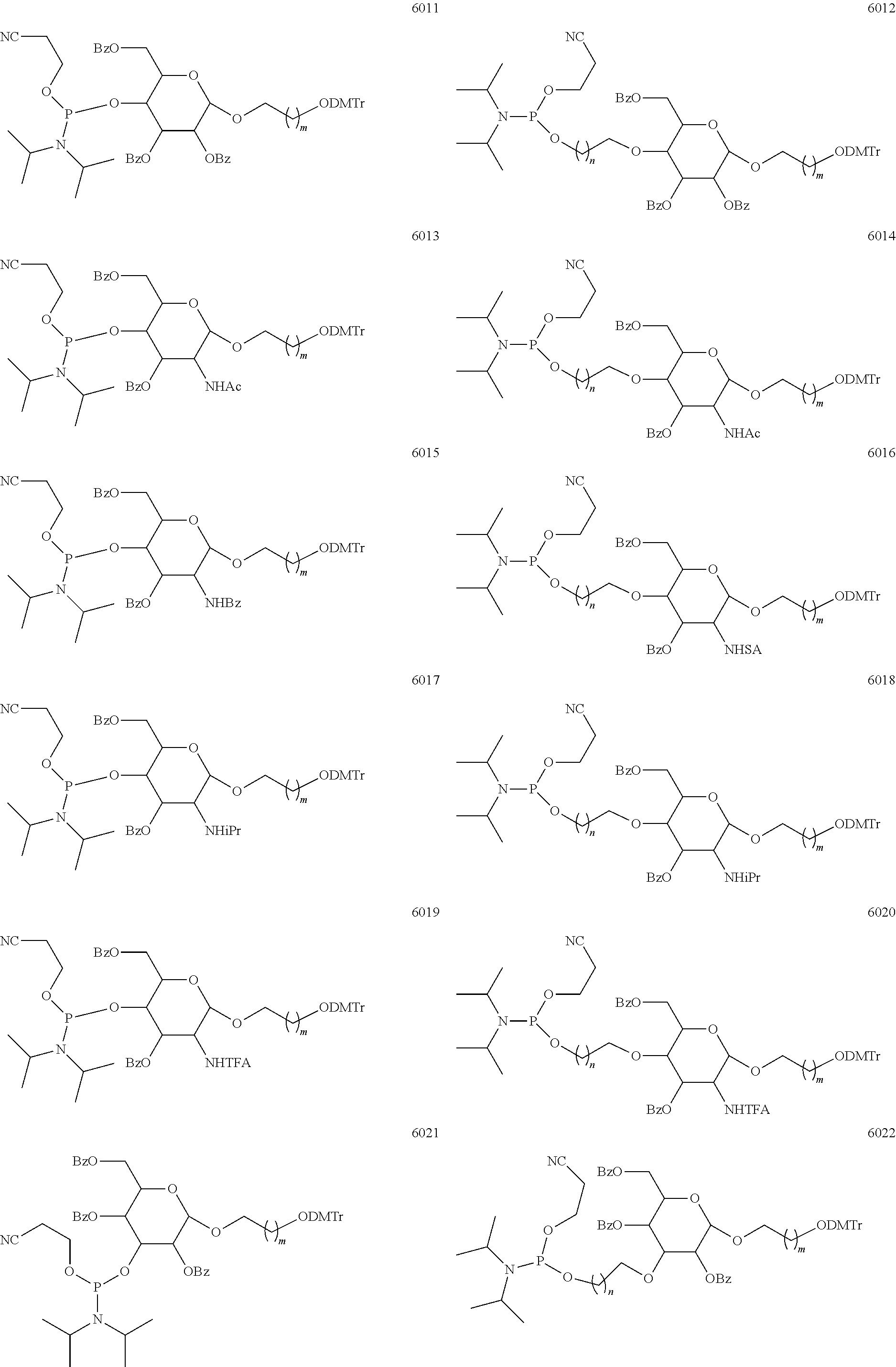

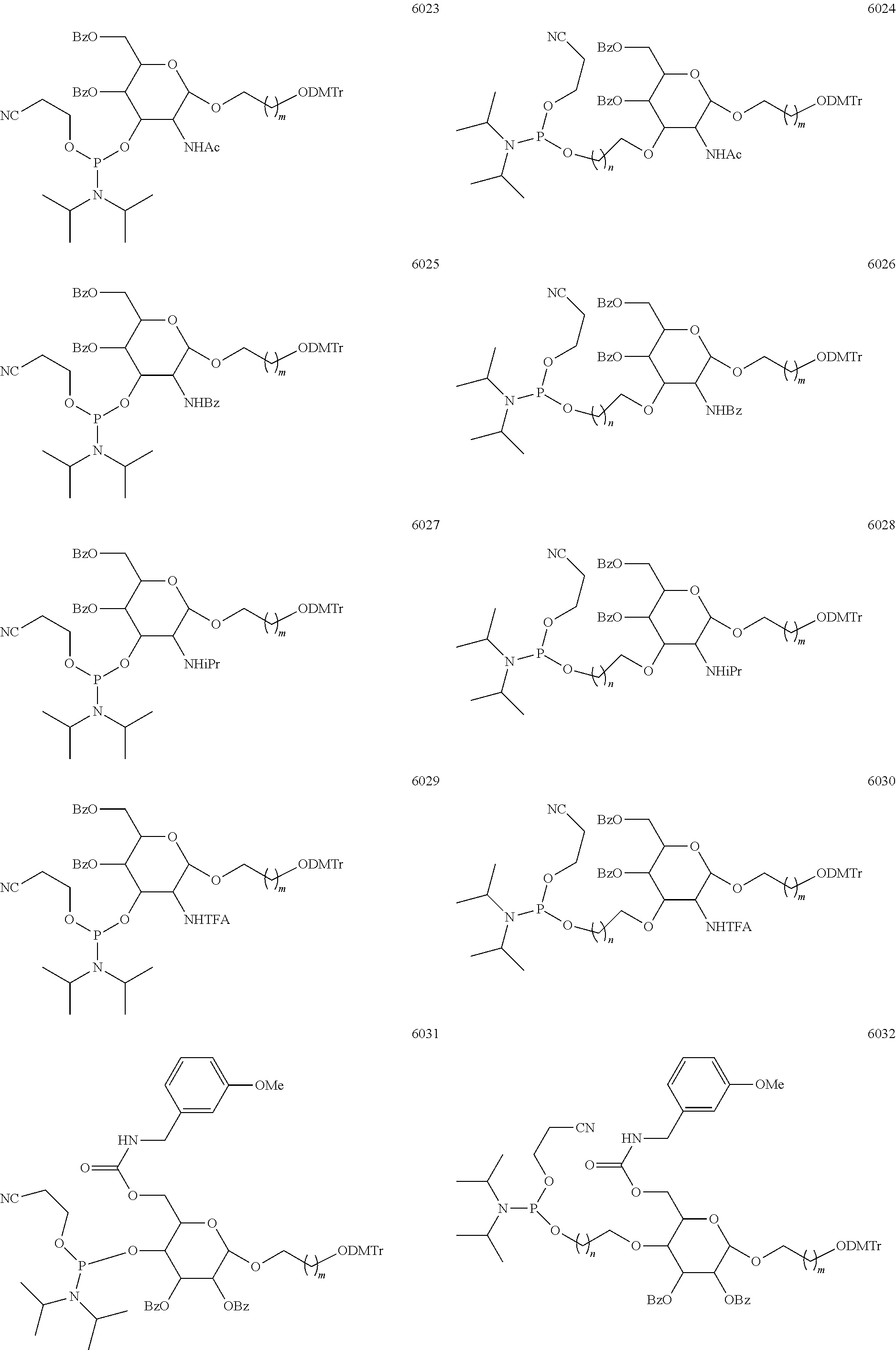

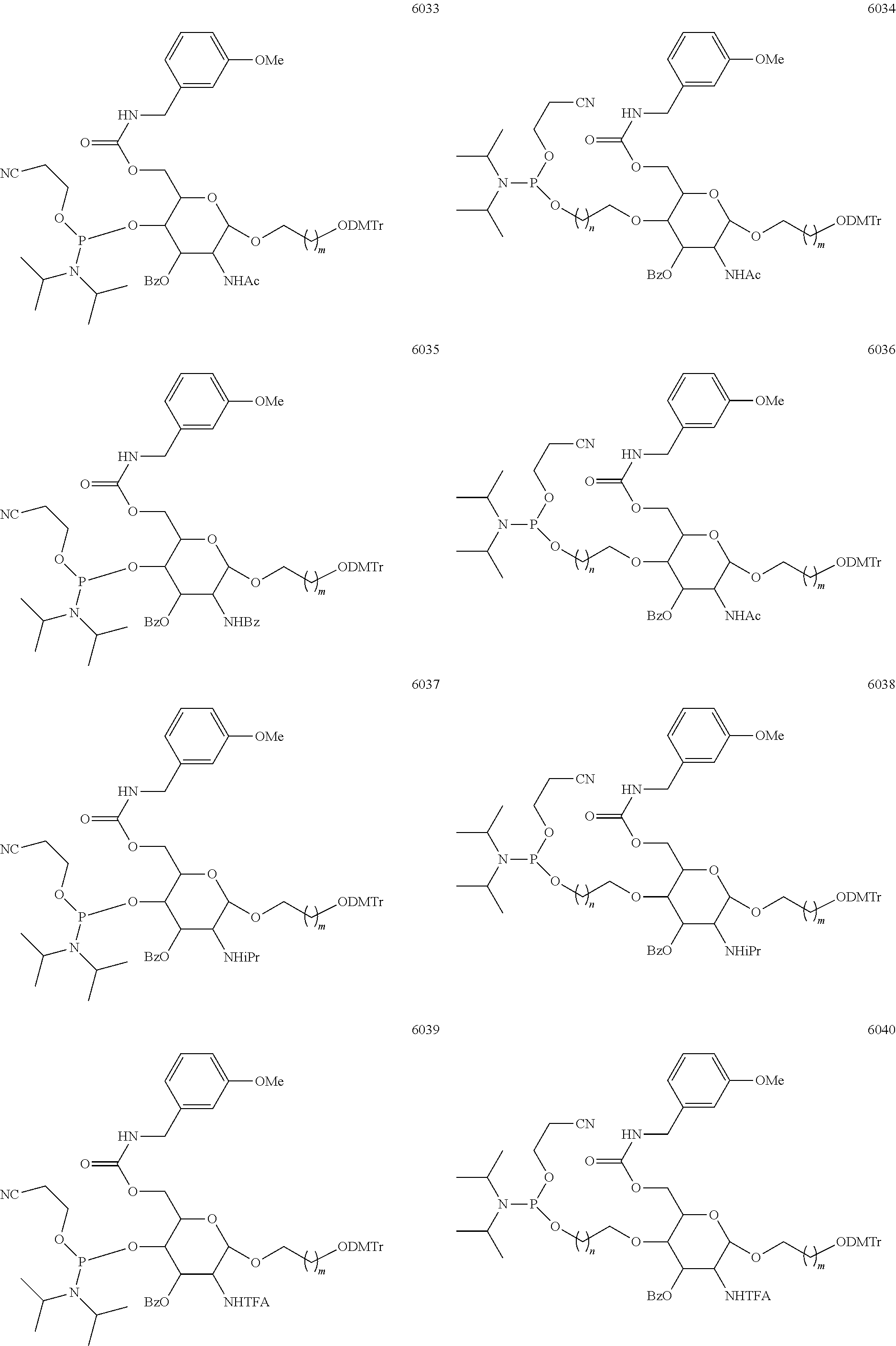

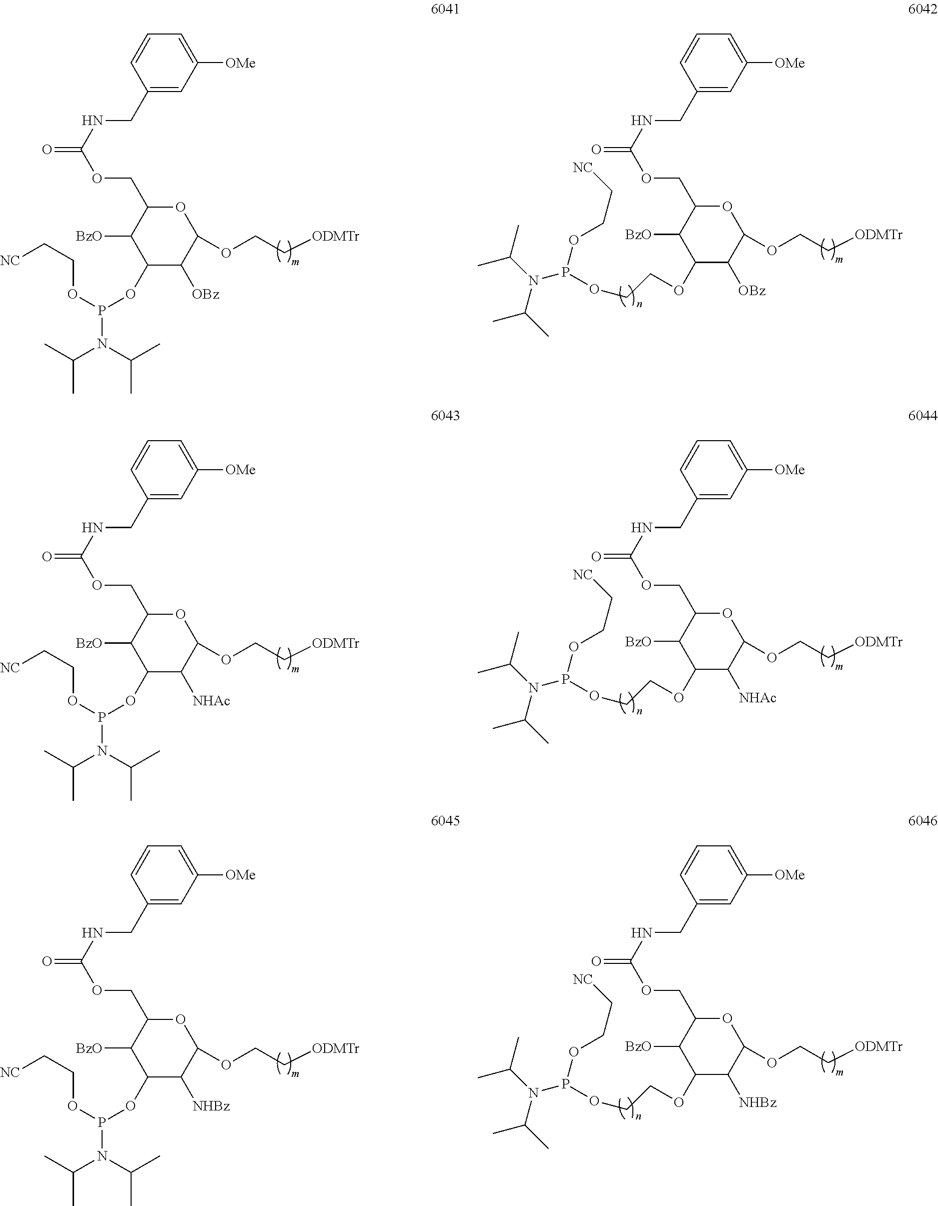

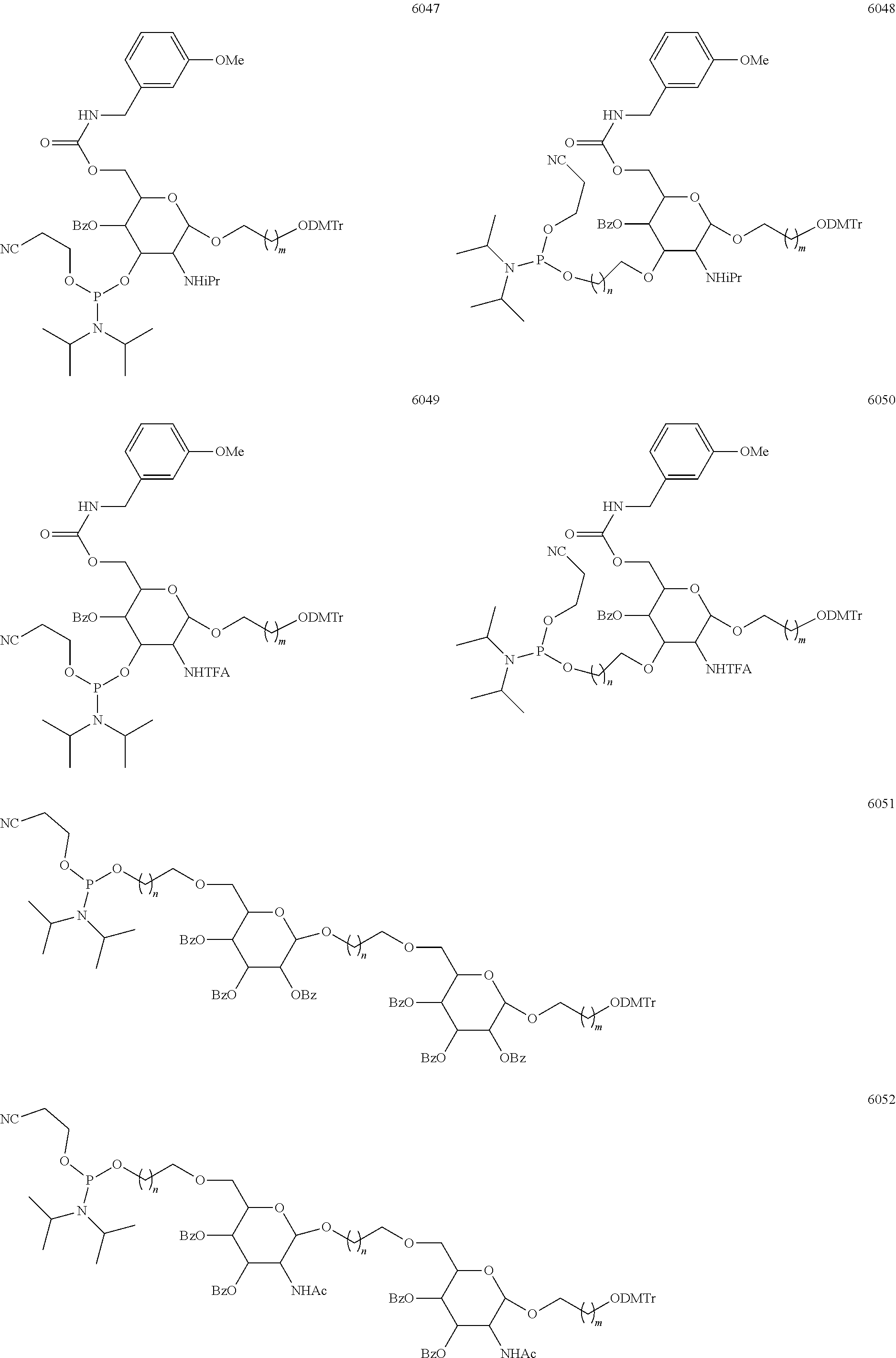

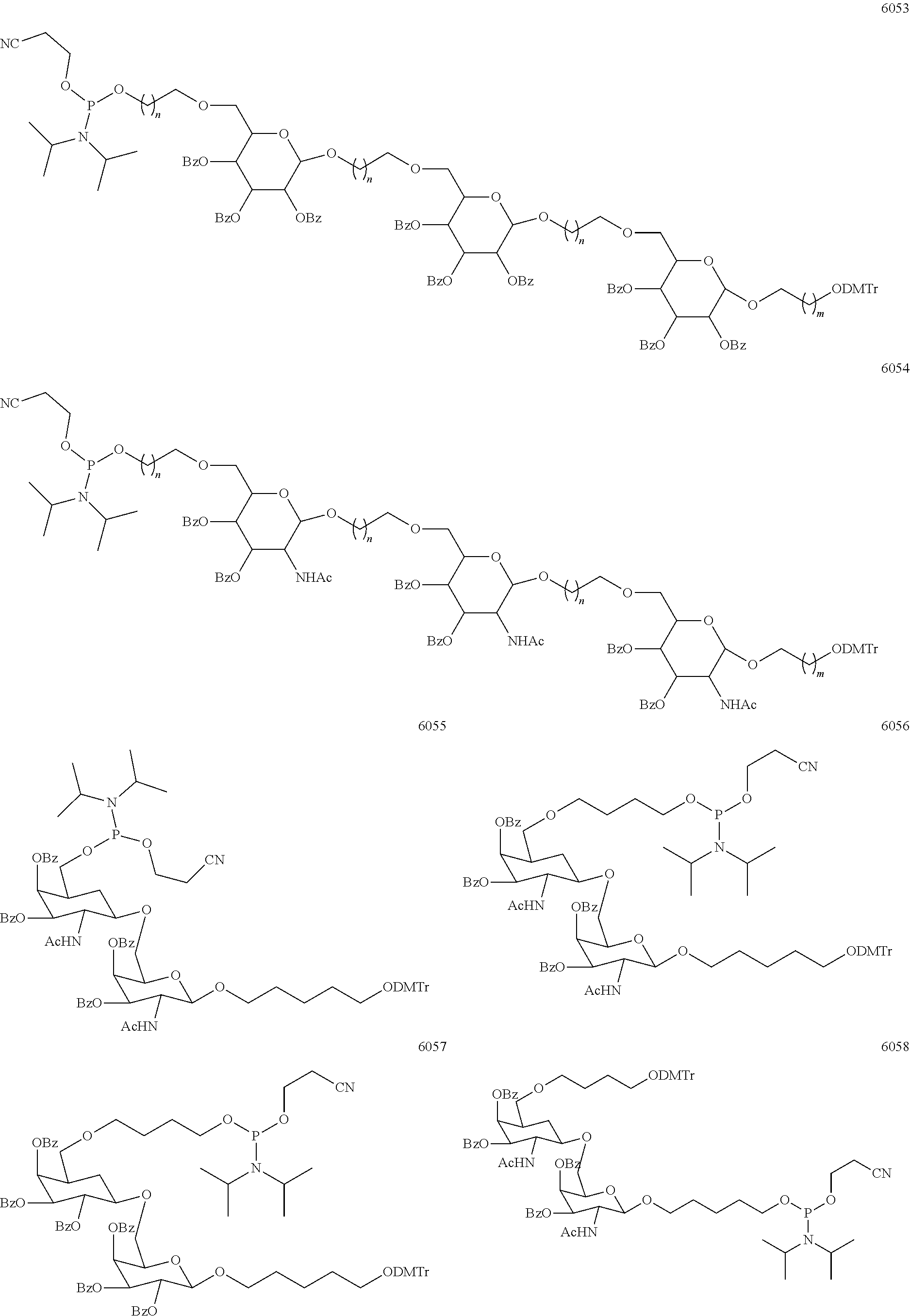

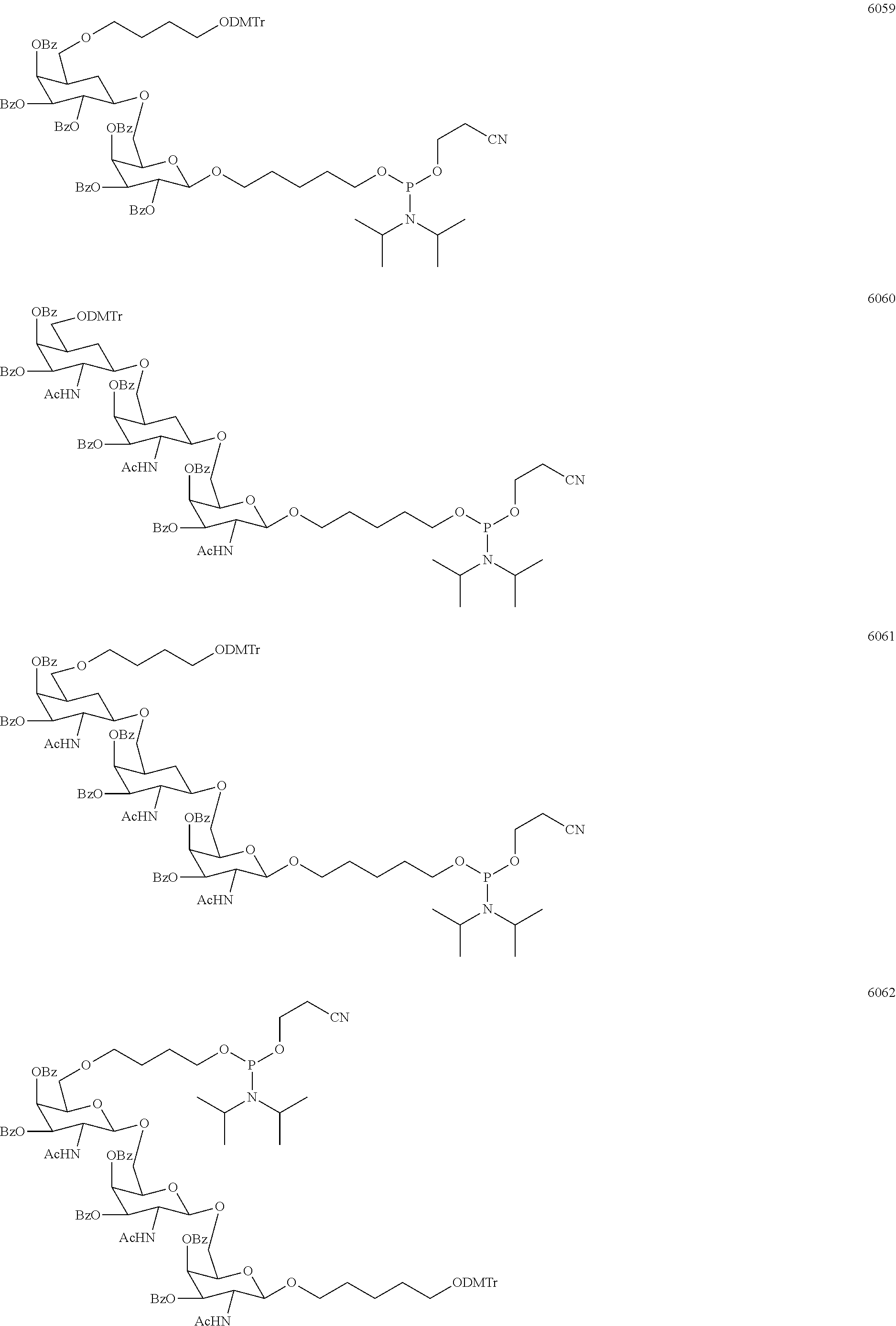

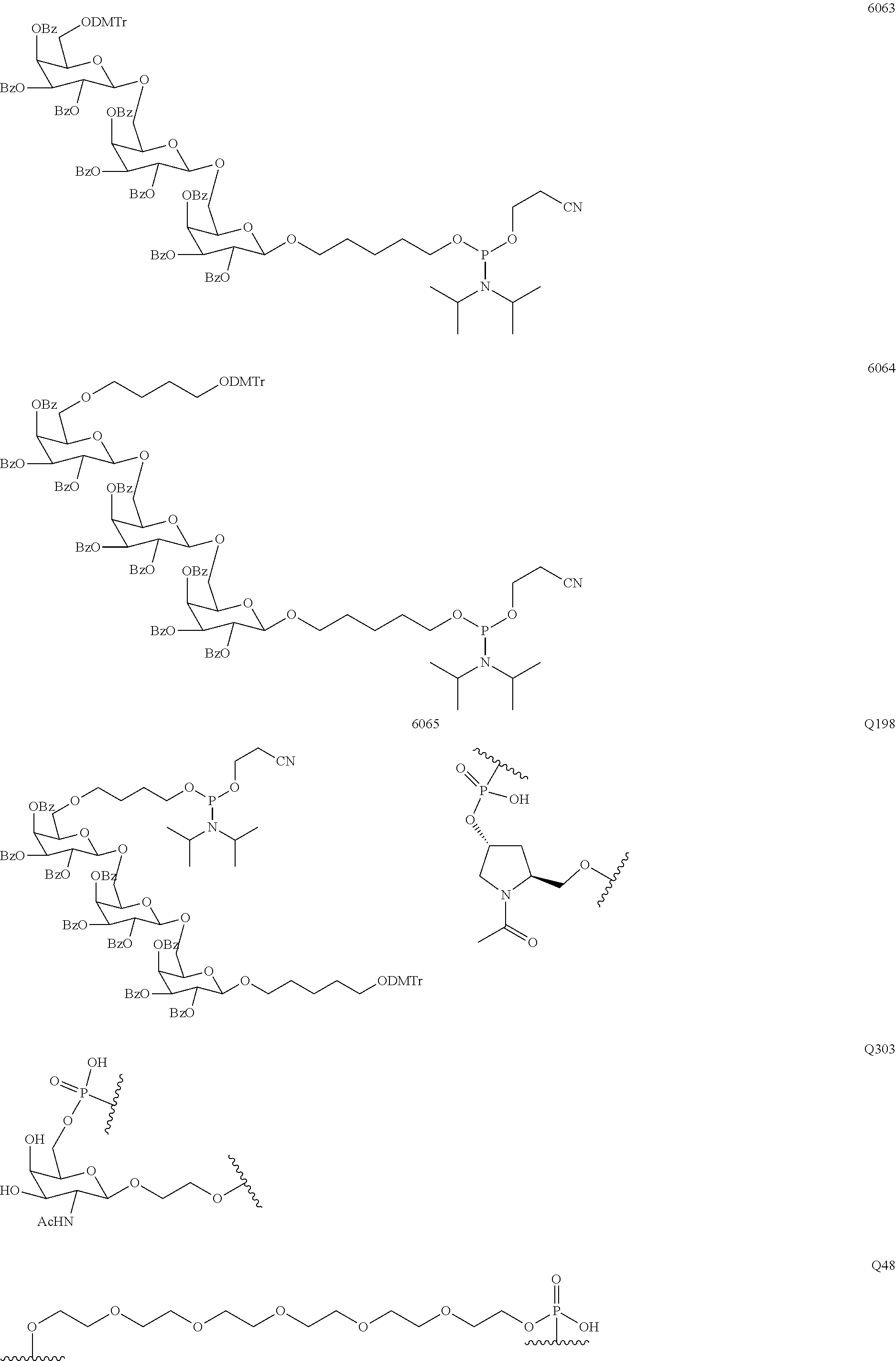

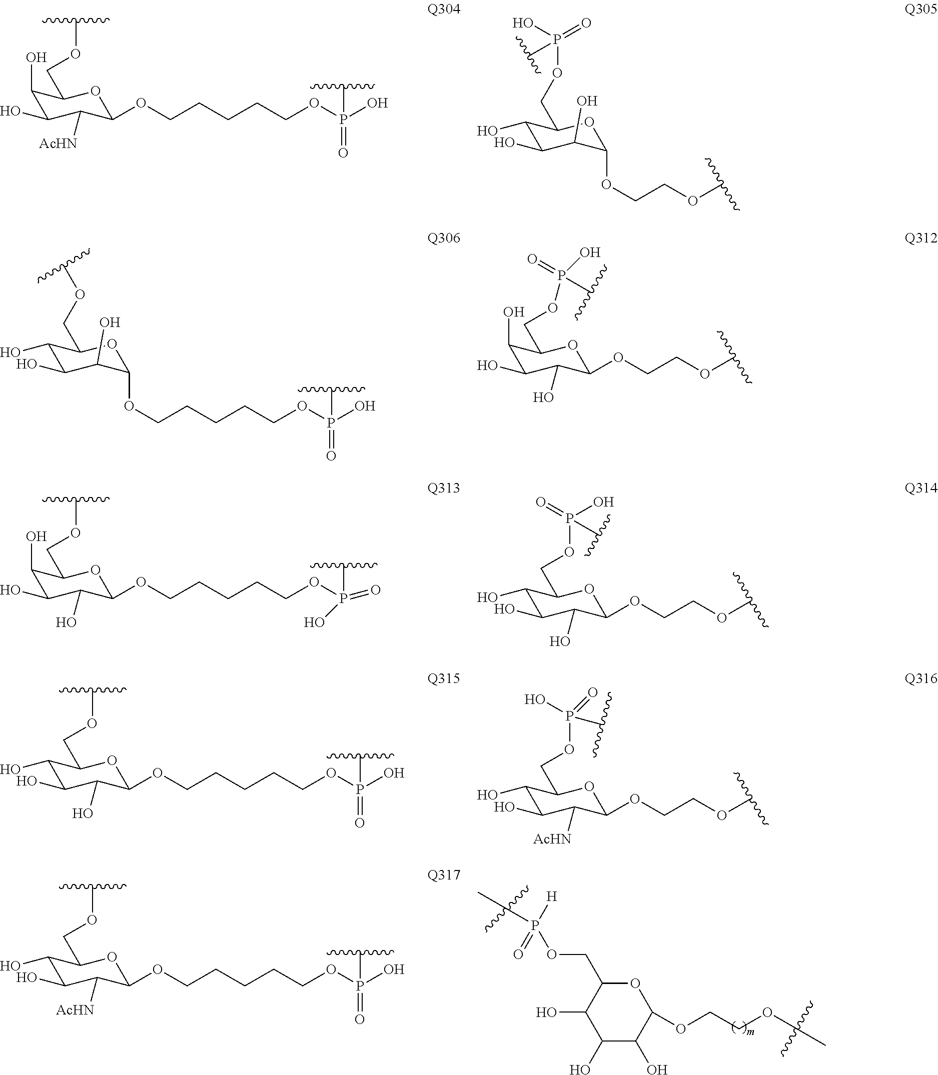

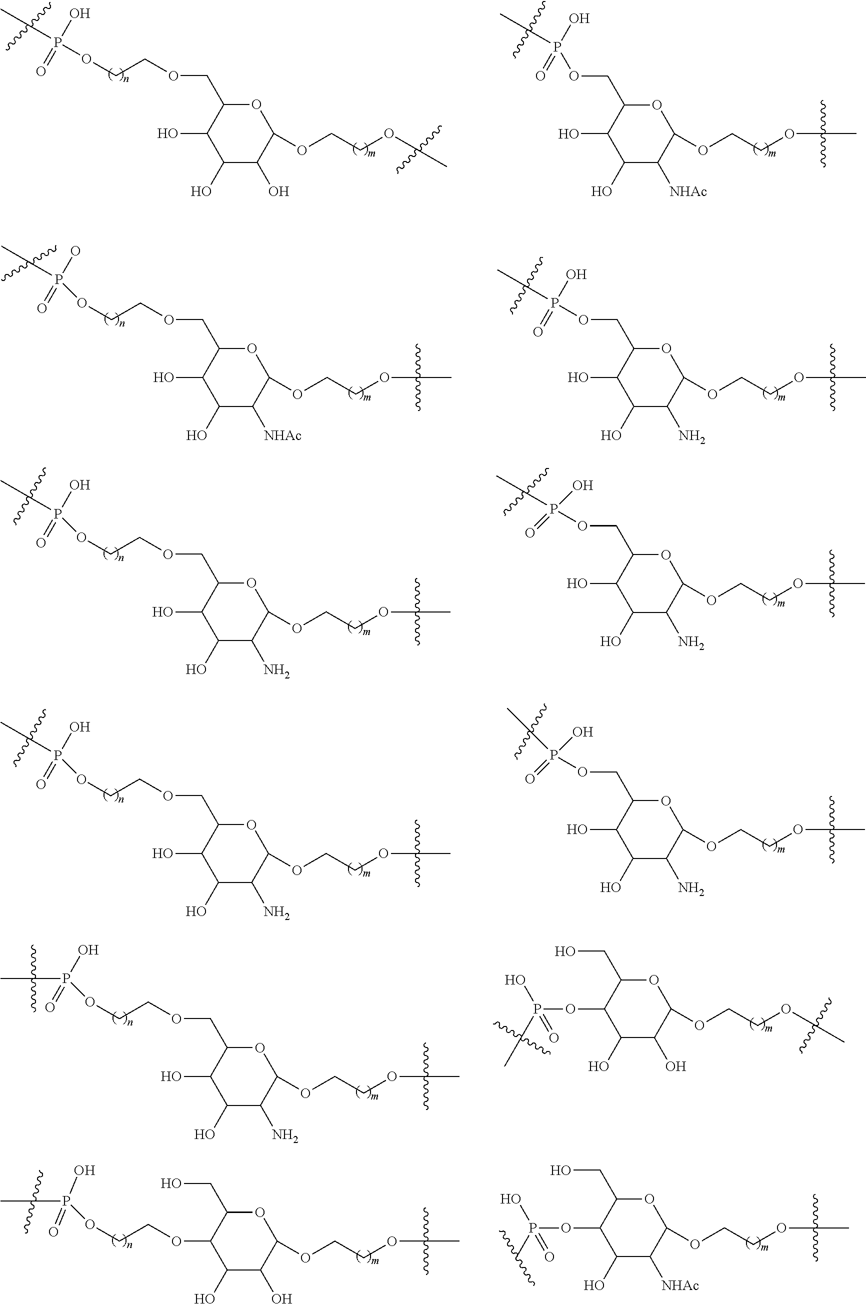

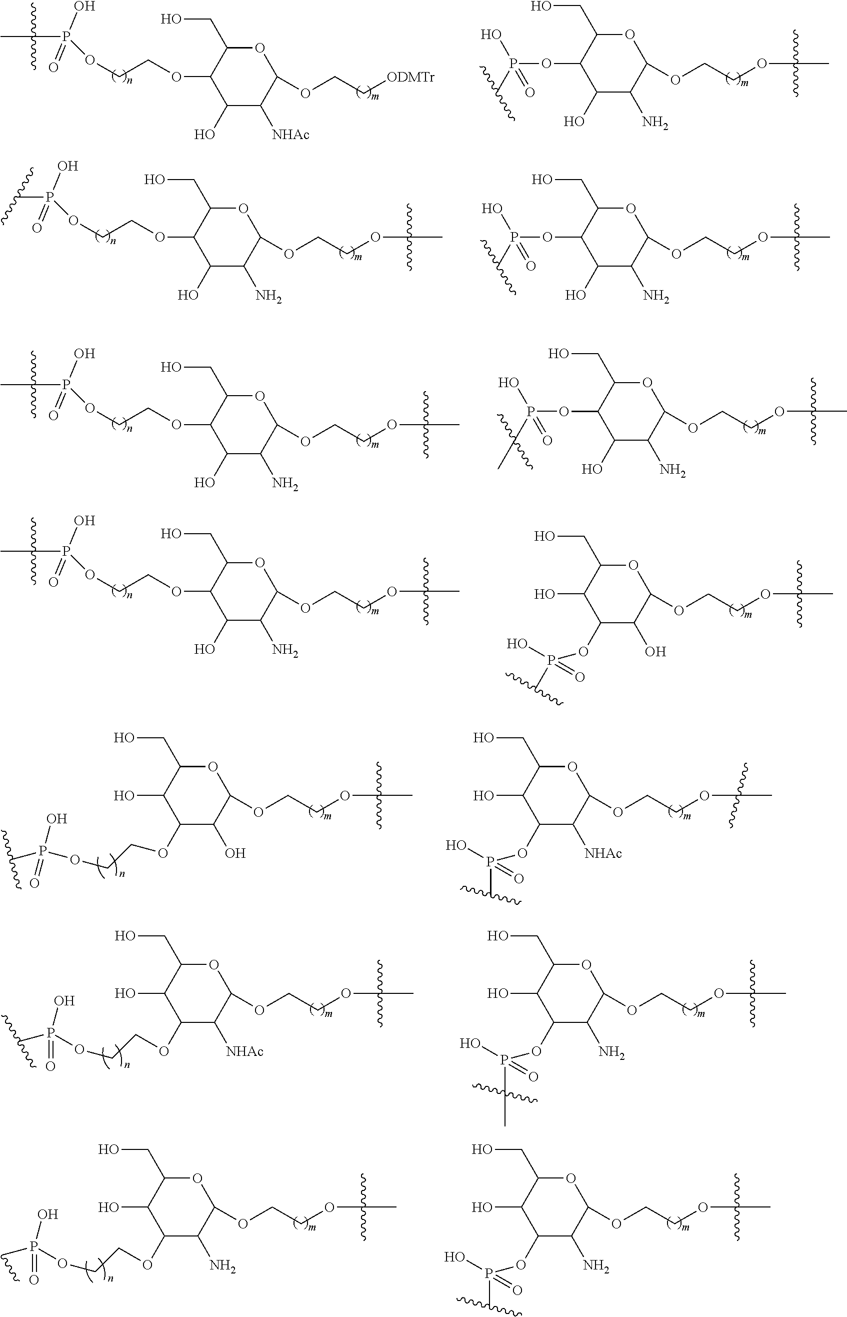

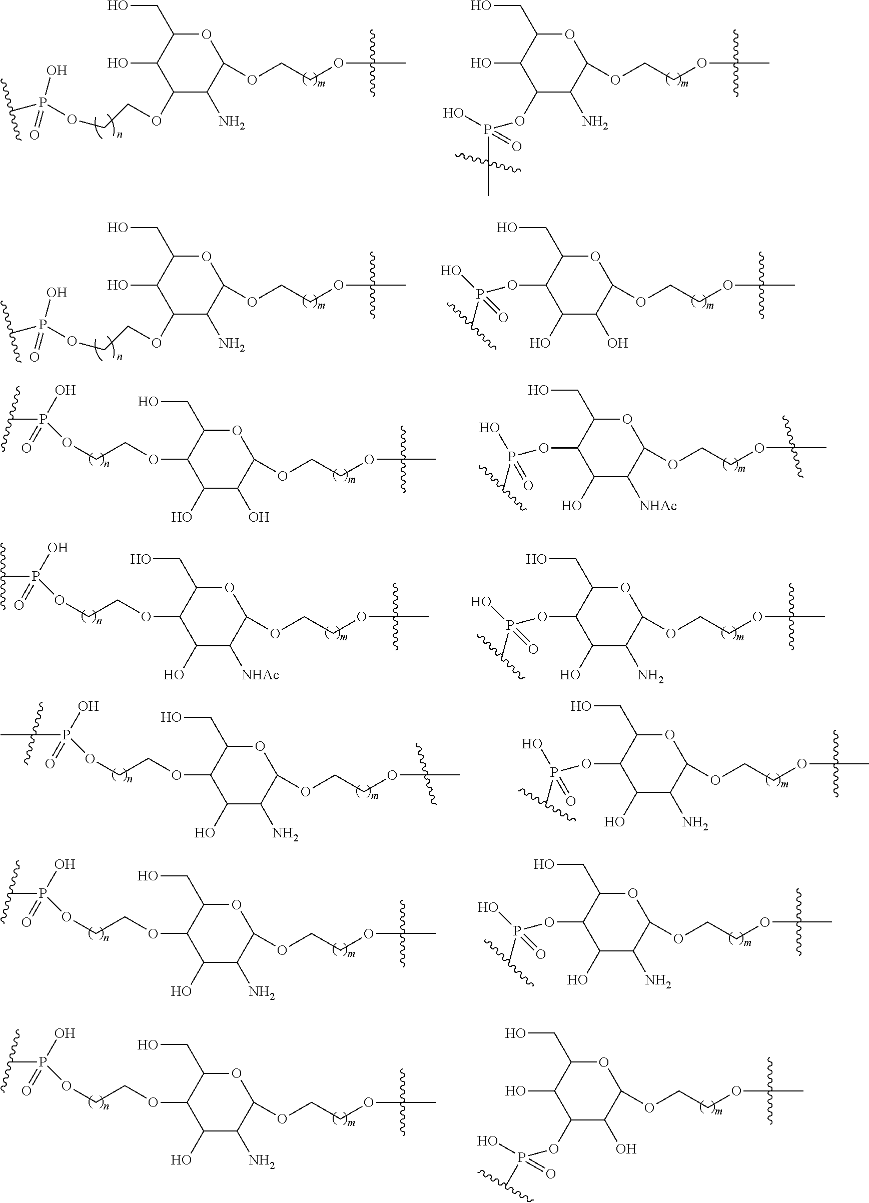

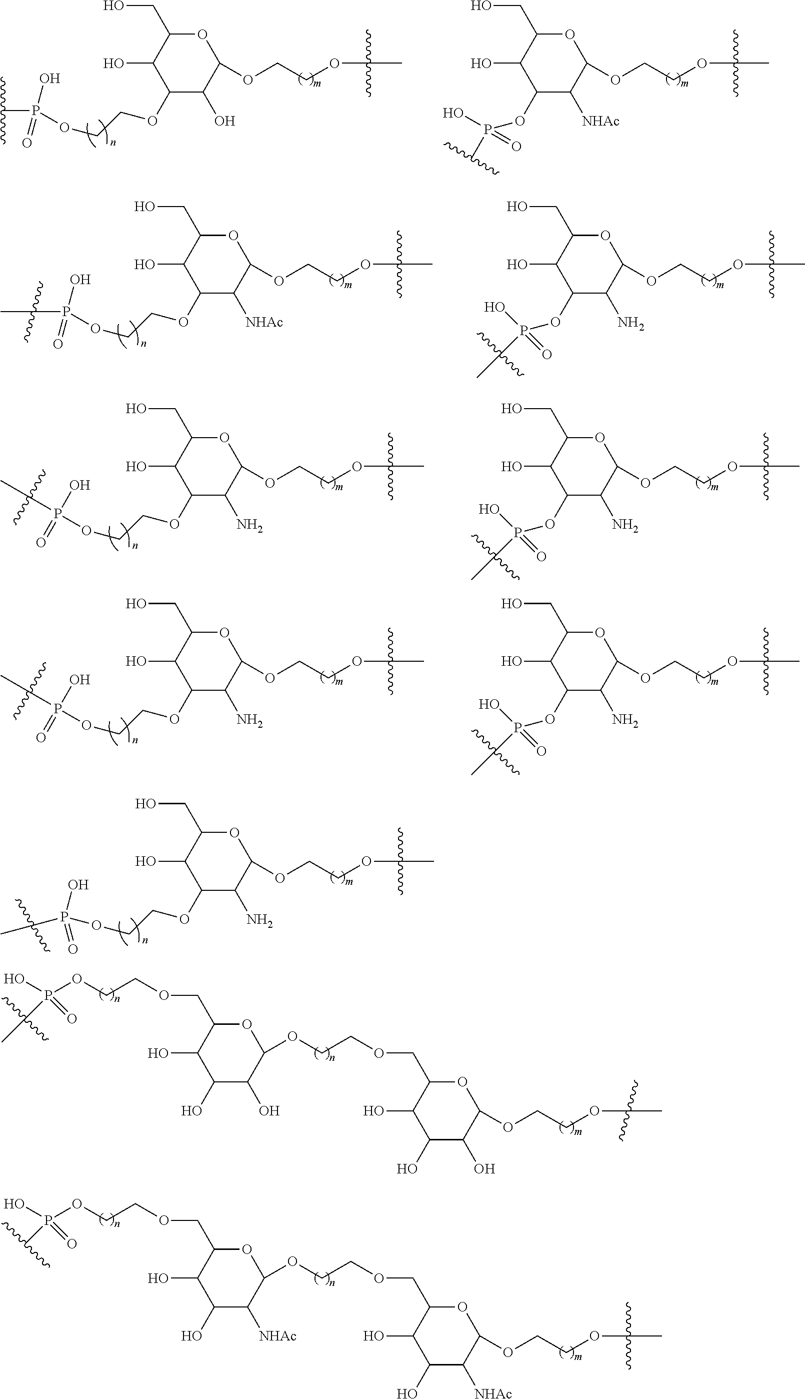

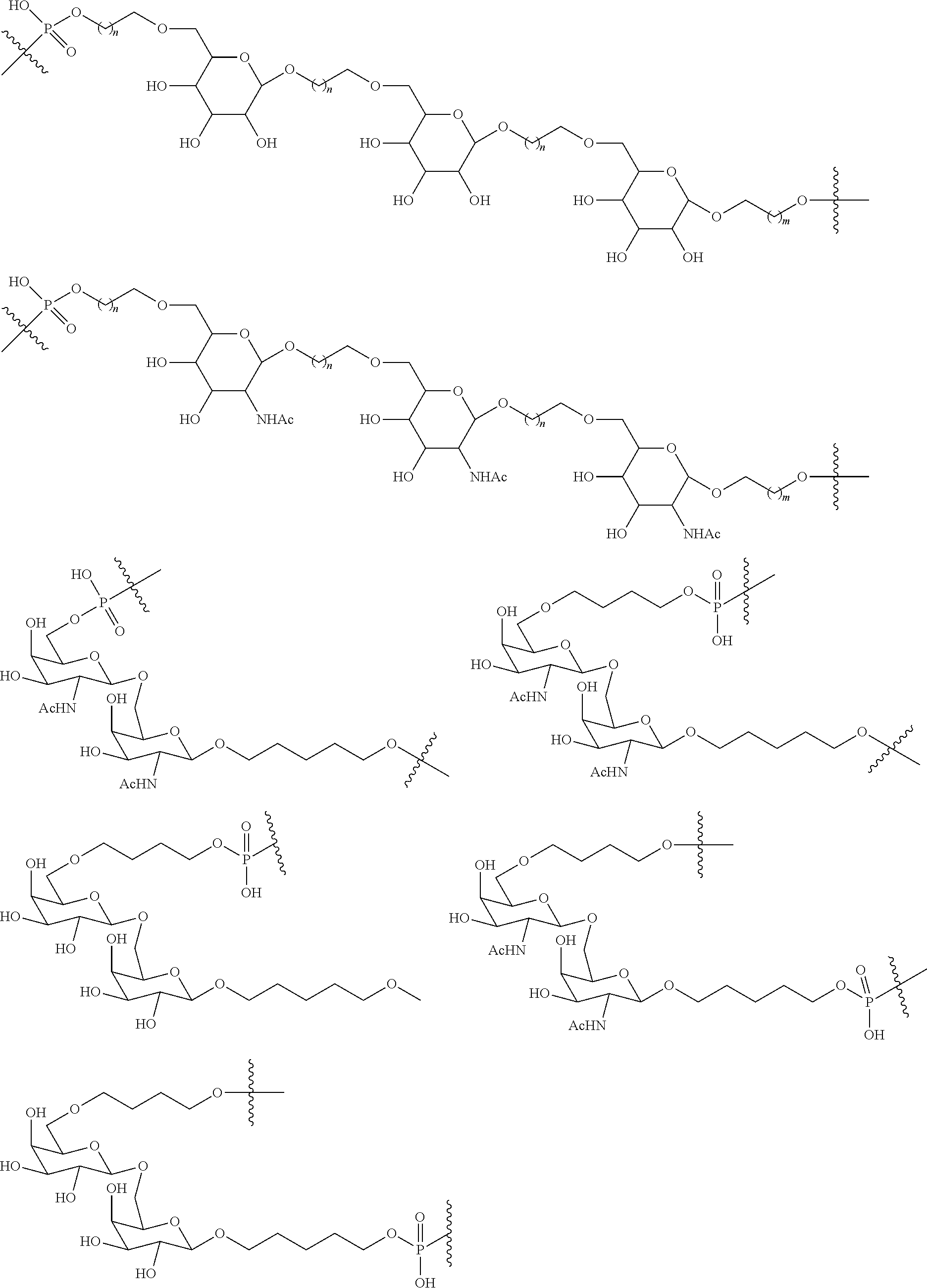

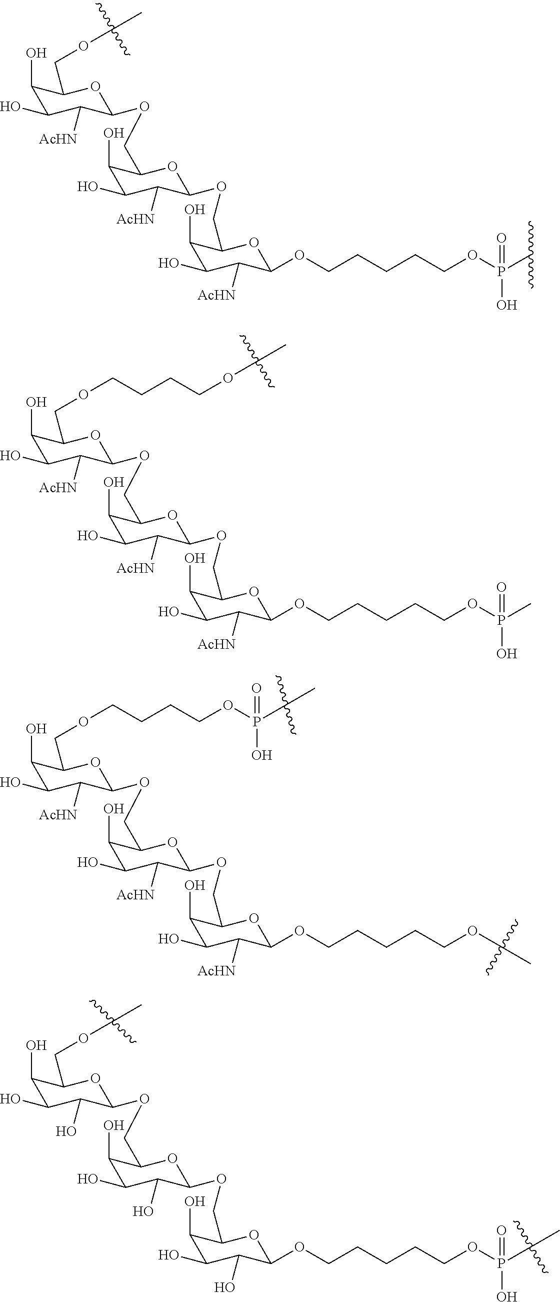

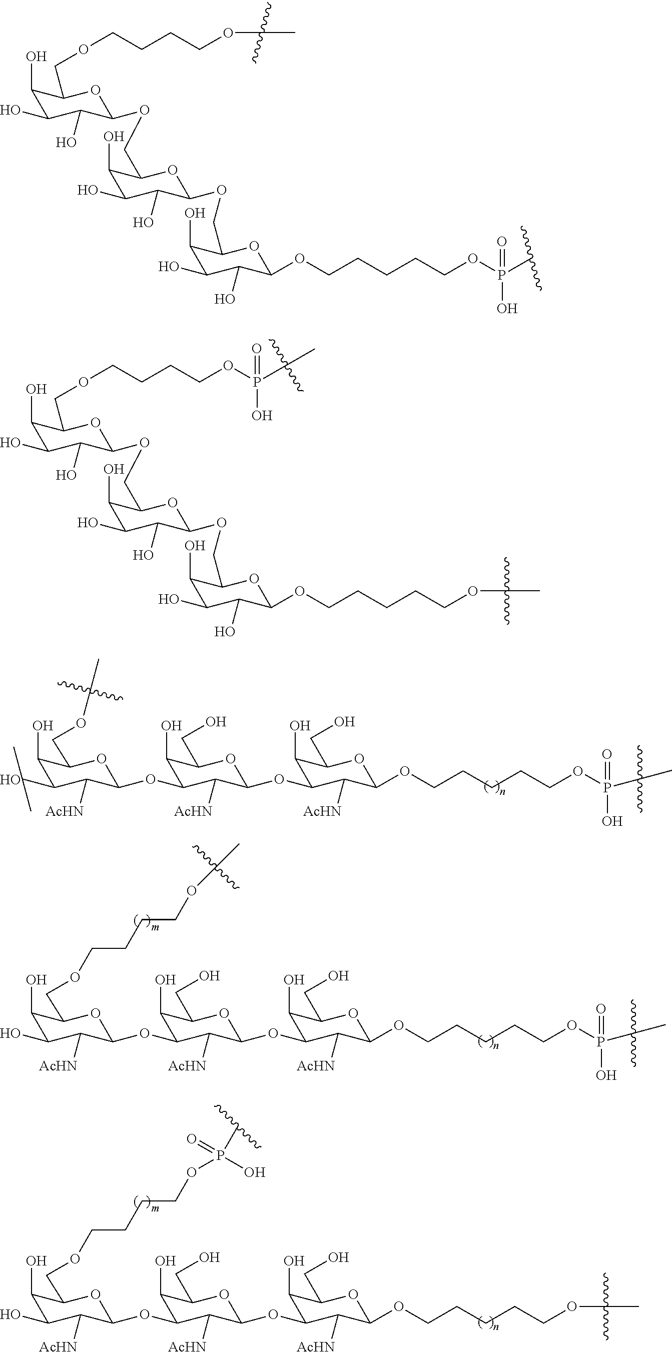

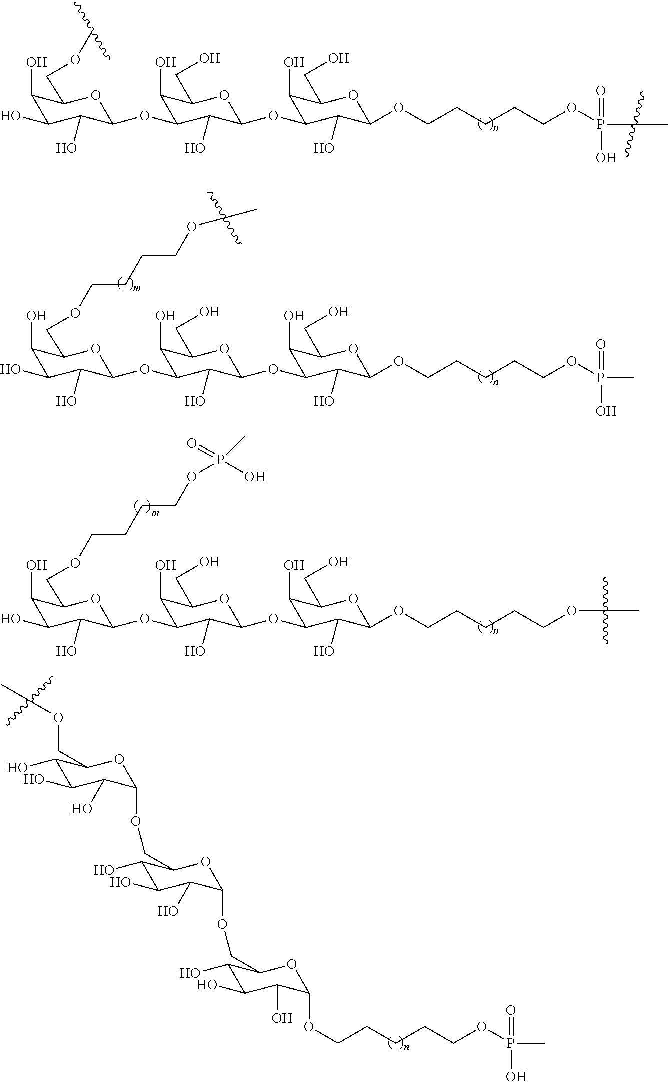

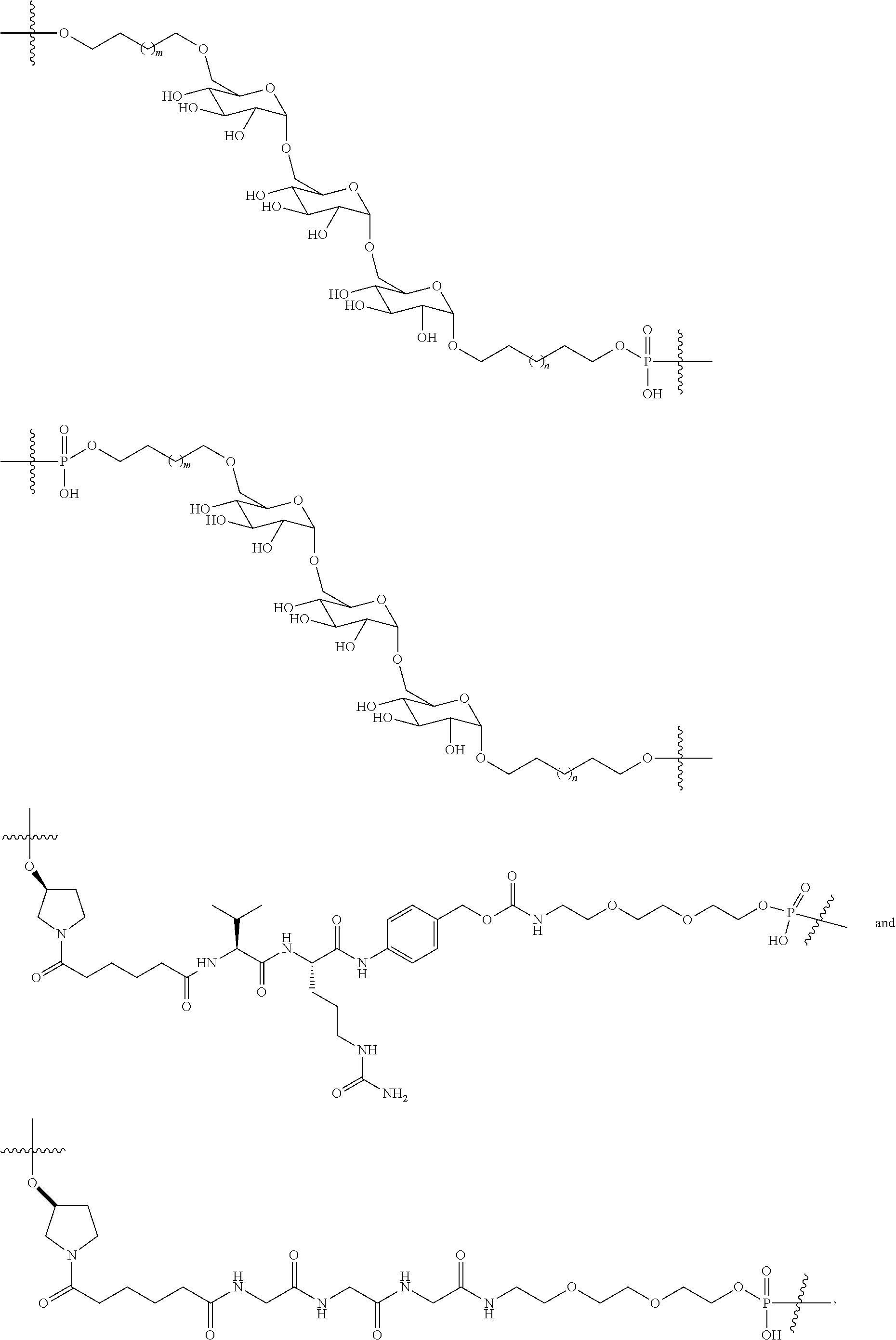

wherein n=1-12 and m=1-12.

More discussion about the biocleavable linkers may be found in WO2018136620, the content of which is incorporated herein by reference in its entirety.

### Carriers

In certain embodiments, the cycling linking moiety of the circular or substantially circular sense (or antisense) strand contains one or more carriers that carry one or more ligands and serve to conjugate the ligand(s) to the sciRNA (or bis-sciRNA).

In certain embodiments, one or more ligands may be conjugated to the sciRNA (or bis-sciRNA) via a carrier, but not as part of the cycling linking moiety.

In certain embodiments, one or more ligands may be conjugated to the effector molecules (e.g., the bis siRNA compounds) via a carrier.

In some embodiments, the carrier may replace one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalinyl. In one embodiment, the acyclic group is a moiety based on a serinol backbone or a diethanolamine backbone.

In some embodiments, the carrier replaces one or more nucleotide(s) in the internal position(s) of the effector molecules (e.g., bis siRNA) or the sciRNA (or bis-sciRNA) agent.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand. The ligand can be directly attached to the carrier or indirectly attached to the carrier by an intervening linker/tether, as described above.

The ligand-conjugated monomer subunit may be the 5′ or 3′ terminal subunit of the effector molecule (e.g., dsRNA) or the sciRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an sciRNA (or bis-sciRNA) agent.

### Sugar Replacement-Based Monomers, e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (In that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

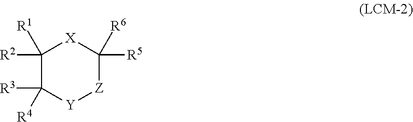

wherein:
X is N(CO)R⁷, NR⁷ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR^{a}, or (CH₂)ₙOR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R^{d}, or R⁷ can be a ligand tethered indirectly to the carrier, e.g., through a tethering moiety, e.g., C₁-C₂₀ alkyl substituted with NR^{c}R^{d}; or
C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or
Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR⁷, Y is CR⁹R¹⁰, and Z is absent; or X is N(CO)R⁷ or NR⁷, Y is CR⁹R¹⁰, and Z is CR¹¹R¹²; or X is N(CO)R⁷ or NR⁷, Y is NR⁸, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

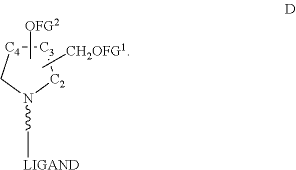

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or —CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

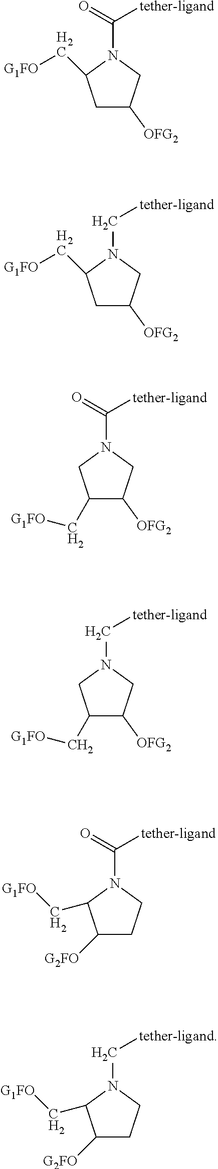

In certain embodiments, the carrier may be based on the piperidine ring system (E), e.g., X is N(CO)R⁷ or NR⁷, Y is CR⁹R¹⁰, and Z is CR¹¹R¹².

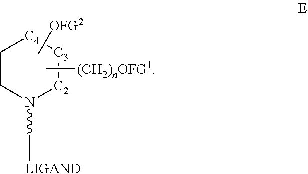

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperidine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

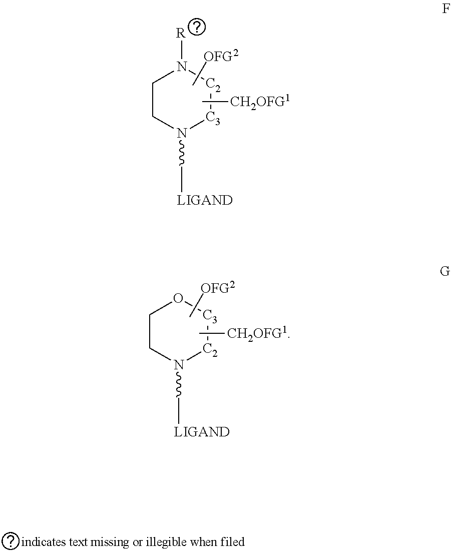

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

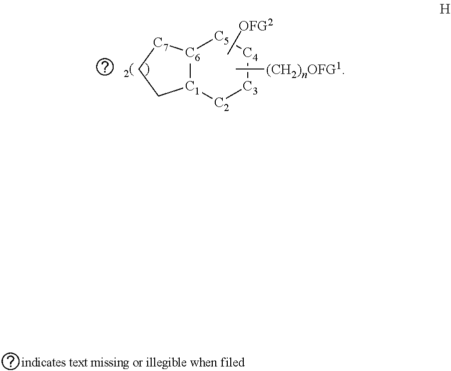

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

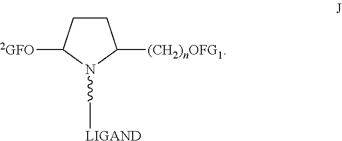

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

### Sugar Replacement-Based Monomers (Acyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

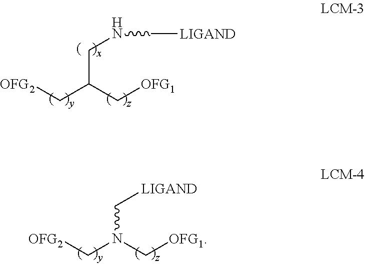

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

In some embodiments, the multi-targeted molecules (e.g., the effector molecules such as bis siRNA or the sciRNA (or bis-sciRNA) agent) comprises one or more ligands conjugated to the 5′ end of a sense nucleotide sequence or the 5′ end of an antisense nucleotide sequence.

In certain embodiments, the ligand is conjugated to the 5′-end of a nucleotide sequence via a carrier and/or linker. In one embodiment, the ligand is conjugated to the 5′-end of a nucleotide sequence via a carrier of a formula:

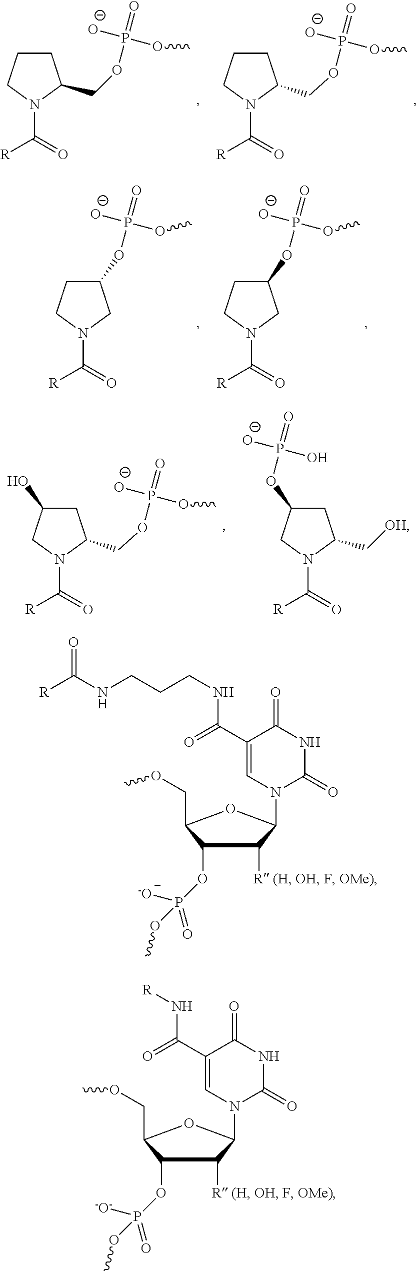

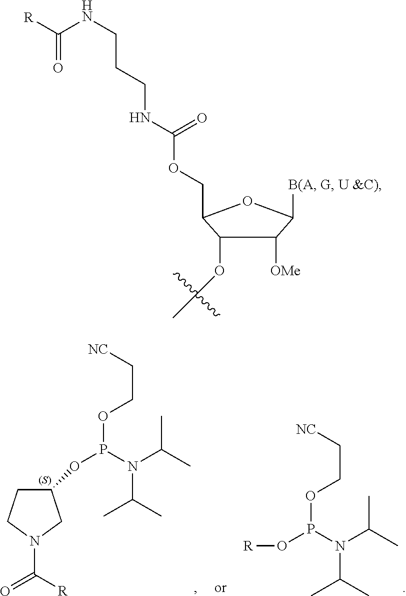

R is a ligand.

In some embodiments, the multi-targeted molecules (e.g., the effector molecules such as bis siRNA or the sciRNA (or bis-sciRNA) agent) comprises one or more ligands conjugated to the 3′ end of a sense nucleotide sequence or the 3′ end of an antisense nucleotide sequence.

In certain embodiments, the ligand is conjugated to the 3′-end of a nucleotide sequence via a carrier and/or linker. In one embodiment, the ligand is conjugated to the 3′-end of a nucleotide sequence of a strand via a carrier of a formula:

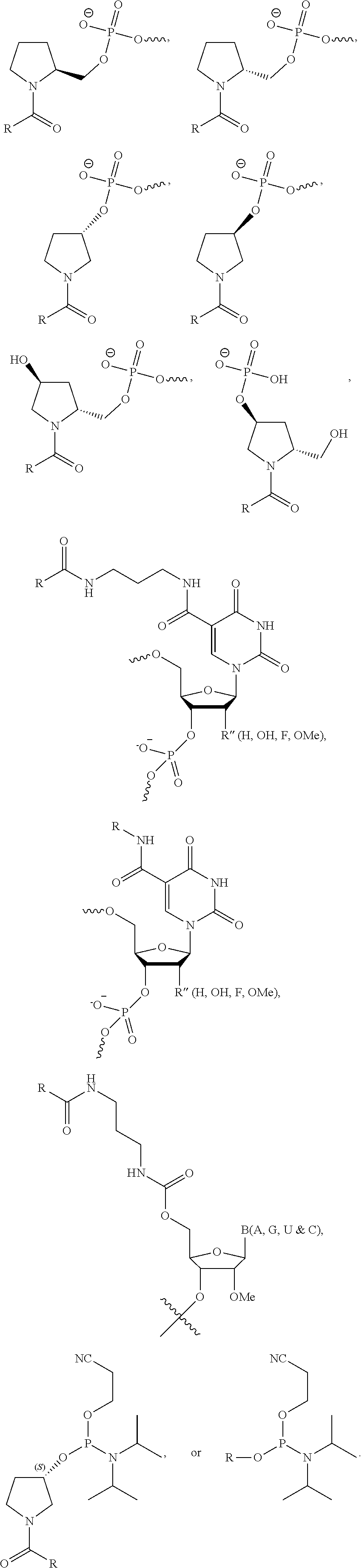

R is a ligand.

In certain embodiments, at least one of the ligands is conjugated to a strand that has a circular or substantially circular structure. In certain embodiments, at least one of the ligands is conjugated to a strand that does not have a circular or substantially circular structure. In one embodiment, at least one of the ligands is conjugated to a strand that has a circular or substantially circular structure, and at least one of the ligands is conjugated to a strand that does not have a circular or substantially circular structure.

In some embodiments, the multi-targeted molecule (e.g., the effector molecules such as bis siRNA or the sciRNA (or bis-sciRNA) agent) comprises one or more ligands conjugated to both ends of a sense nucleotide sequence. In some embodiments, the multi-targeted molecule (e.g., the effector molecules such as bis siRNA or the sciRNA (or bis-sciRNA) agent) comprises one or more ligands conjugated to both ends of an antisense nucleotide sequence.

In some embodiments, the multi-targeted molecule (e.g., the effector molecules such as bis siRNA or the sciRNA (or bis-sciRNA) agent) comprises one or more ligands conjugated to the 5′ end or 3′ end of a sense nucleotide sequence, and one or more ligands conjugated to the 5′ end or 3′ end of an antisense nucleotide sequence.

In some embodiments, the ligand is conjugated to a strand via one or more linkers (tethers) and/or a carrier. In one embodiment, the ligand is conjugated to a strand via one or more linkers (tethers).

In one embodiment, the ligand is conjugated to the 5′ end or 3′ end of a sense nucleotide sequence or antisense nucleotide sequence via a cyclic carrier, optionally via one or more intervening linkers (tethers).

In some embodiments, the ligand is conjugated to one or more internal positions on at least one nucleotide sequence. Internal positions of a nucleotide sequence refer to the nucleotide on any position of the nucleotide sequence, except the terminal position from the 3′ end and 5′ end of the nucleotide sequence (e.g., excluding 2 positions: position 1 counting from the 3′ end and position 1 counting from the 5′ end).

In one embodiment, the ligand is conjugated to one or more internal positions on at least one nucleotide sequence, which include all positions except the terminal two positions from each end of the nucleotide sequence (e.g., excluding 4 positions: positions 1 and 2 counting from the 3′ end and positions 1 and 2 counting from the 5′ end). In one embodiment, the lipophilic moiety is conjugated to one or more internal positions on at least one nucleotide sequence, which include all positions except the terminal three positions from each end of the nucleotide sequence (e.g., excluding 6 positions: positions 1, 2, and 3 counting from the 3′ end and positions 1, 2, and 3 counting from the 5′ end).

In one embodiment, the ligand is conjugated to one or more internal positions on at least one nucleotide sequence, except the cleavage site region of a sense nucleotide sequence, for instance, the ligand is not conjugated to positions 9-12 counting from the 5′-end of the sense nucleotide sequence, for example, the ligand is not conjugated to positions 9-11 counting from the 5′-end of the sense nucleotide sequence. Alternatively, the internal positions exclude positions 11-13 counting from the 3′-end of the sense nucleotide sequence.

In one embodiment, the ligand is conjugated to one or more internal positions on at least one nucleotide sequence, which exclude the cleavage site region of an antisense nucleotide sequence. For instance, the internal positions exclude positions 12-14 counting from the 5′-end of the antisense nucleotide sequence.

In one embodiment, the ligand is conjugated to one or more internal positions on at least one nucleotide sequence, which exclude positions 11-13 on a sense nucleotide sequence, counting from the 3′-end, and positions 12-14 on an antisense nucleotide sequence, counting from the 5′-end.

In one embodiment, one or more ligands are conjugated to one or more of the following internal positions: positions 4-8 and 13-18 on a sense nucleotide sequence, and positions 6-10 and 15-18 on an antisense nucleotide sequence, counting from the 5′end of each nucleotide sequence.

In one embodiment, one or more ligands are conjugated to one or more of the following internal positions: positions 5, 6, 7, 15, and 17 on a sense nucleotide sequence, and positions 15 and 17 on an antisense sequence, counting from the 5′end of each nucleotide sequence.

In some embodiments, the ligand is conjugated to a nucleobase, sugar moiety, or internucleosidic linkage of the multi-targeted molecule (e.g., the effector molecules such as bis siRNA or the sciRNA (or bis-sciRNA) agent).

### Ligands

In certain embodiments, the multi-targeted molecule (e.g., the effector molecules such as bis siRNA, or the sciRNA agent (or bis-scriRNA)) is further modified by covalent attachment of one or more conjugate groups. In general, conjugate groups modify one or more properties of the attached effector molecules (e.g., bis siRNA) or sciRNA agent (or bis-scriRNA) including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the multi-targeted molecule (e.g., the effector molecules such as bis siRNA or the sciRNA (or bis-sciRNA) agent) further comprises a targeting ligand that targets a receptor which mediates delivery to a specific CNS tissue. These targeting ligands can be conjugated in combination with the lipophilic moiety to enable specific intrathecal and systemic delivery.

Exemplary targeting ligands that targets the receptor mediated delivery to a CNS tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

In some embodiments, the multi-targeted molecule (e.g., the effector molecules such as bis siRNA or the sciRNA (or bis-sciRNA) agent) further comprises a targeting ligand that targets a receptor which mediates delivery to a specific ocular tissue. These targeting ligands can be conjugated in combination with the lipophilic moiety to enable specific intravitreal and systemic delivery. Exemplary targeting ligands that targets the receptor mediated delivery to a ocular tissue are lipophilic ligands such as all-trans retinol (which targets the retinoic acid receptor); RGD peptide (which targets retinal pigment epithelial cells), such as H-Gly-Arg-Gly-Asp-Ser-Pro-Lys-Cys-OH or Cyclo(-Arg-Gly-Asp-D-Phe-Cys; LDL receptor ligands; and carbohydrate based ligands (which targets endothelial cells in posterior eye).

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

As used herein the term “targeting ligand” refers to any molecule that provides an enhanced affinity for a selected target, e.g., a cell, cell type, tissue, organ, region of the body, or a compartment, e.g., a cellular, tissue or organ compartment. For example, targeting ligands for the CNS include the lipophilic ligands herein, such as C16-modifications.

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to, AALEALAEALEALAEALEALAEAAAAGGC (GALA); AALAEALAEALAEALAEALAEALAAAAGGC (EALA); ALEALAEALEALAEA; GLFEAIEGFIENGWEGMIWDYG (INF-7); GLFGAIAGFIENGWEGMIDGWYG (Inf HA-2); GLFEAIEGFIENGWEGMIDGWYGCGLFEAIEGFIENGWEGMID GWYGC (diINF-7); GLFEAIEGFIENGWEGMIDGGCGLFEAIEGFIENGWEGMIDGGC (diINF-3); GLFGALAEALAEALAEHLAEALAEALEALAAGGSC (GLF); GLFEAIEGFIENGWEGLAEALAEALEALAAGGSC (GALA-INF3); GLF EAI EGFI ENGW EGnI DG K GLF EAI EGFI ENGW EGnI DG (INF-5, n is norleucine); LFEALLELLESLWELLLEA (JTS-1); GLFKALLKLLKSLWKLLLKA (ppTG1); GLFRALLRLLRSLWRLLLRA (ppTG20); WEAKLAKALAKALAKHLAKALAKALKACEA (KALA); GLFFEAIAEFIEGGWEGLIEGC (HA); GIGAVLKVLTTGLPALISWIKRKRQQ (Melittin); H₅WYG; and CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also refered to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to, RQIKIWFQNRRMKWKK (penetratin); GRKKRRQRRRPPQC (Tat fragment 48-60); GALFLGWLGAAGSTMGAWSQPKKKRKV (signal sequence based peptide); LLIILRRRIRKQAHAHSK (PVEC); GWTLNSAGYLLKINLKALAALAKKIL (transportan); KLALKLALKALKAALKLA (amphiphilic model peptide); RRRRRRRRR (Arg9); KFFKFFKFFK (Bacterial cell wall permeating peptide); LLGDFFRKSKEKIGKEFKRIVQRIKDFLRNLVPRTES (LL-37); SWLSKTAKKLENSAKKRISEGIAIAIQGGPR (cecropin P1); ACYCRIPACIAGERRYGTCIYQGRLWAFCC (α-defensin); DHYNCVSSGGQCLYSACPIFTKIQGTCYRGKAKCCK (β-defensin); RRRPRPPYLPRPRPPPFFPPRLPPRIPPGFPPRFPPRFPGKR-NH2 (PR-39); ILPWKWPWWPWRR-NH2 (indolicidin); AAVALLPAVLLALLAP (RFGF); AALLPVLLAAP (RFGF analogue); and RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands. For example, targeting ligands for the CNS include the lipophilic ligands herein, such as C16-modifications.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, scuh as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the effector molecule (e.g., bis siRNA) or the sciRNA (or bis-sciRNA) agent. In some embodiments, the ligand can be incorporated via coupling to a “precursor” monomer after said “precursor” monomer has been incorporated into a component of the effector molecule (e.g., bis siRNA) or the sciRNA (or bis-sciRNA) agent. For example, a monomer having, e.g., an amino-terminated tether (i.e., having no associated ligand), e.g., monomer-linker-NH₂ can be incorporated into into a component of the effector molecule (e.g., bis siRNA) or the sciRNA (or bis-sciRNA). In a subsequent operation, i.e., after incorporation of the precursor monomer into a component of the effector molecule (e.g., bis siRNA) or the sciRNA (or bis-sciRNA), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the effector molecule (e.g., bis siRNA) or the sciRNA (or bis-sciRNA) agent. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In some embodiments, the multi-targeted molecule (e.g., the effector molecules such as bis siRNA or the sciRNA (or bis-sciRNA) agent) further comprises one or more targeting ligands that target a liver tissue. In some embodiments, at least one of the targeting ligands is a carbohydrate-based ligand. In some embodiments, the carbohydrate-based ligand is an ASGPR ligand. In one embodiment, at least one of the targeting ligands is a GalNAc-based conjugate.

In certain embodiments, the multi-targeted molecule (e.g., the effector molecules such as bis siRNA or the sciRNA (or bis-sciRNA) agent) further comprises a ligand having a structure shown below:

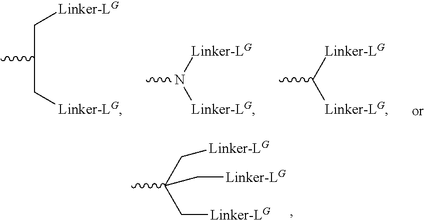

wherein:
L^{G} is independently for each occurrence a ligand, e.g., carbohydrate, e.g. monosaccharide, disaccharide, trisaccharide, tetrasaccharide, polysaccharide; and
Z′, Z″, Z′″ and Z″″ are each independently for each occurrence O or S.

In certain embodiments, the multi-targeted molecule (e.g., the effector molecules such as bis siRNA or the sciRNA (or bis-sciRNA) agent) comprises a ligand of Formula (II), (III), (IV) or (V):

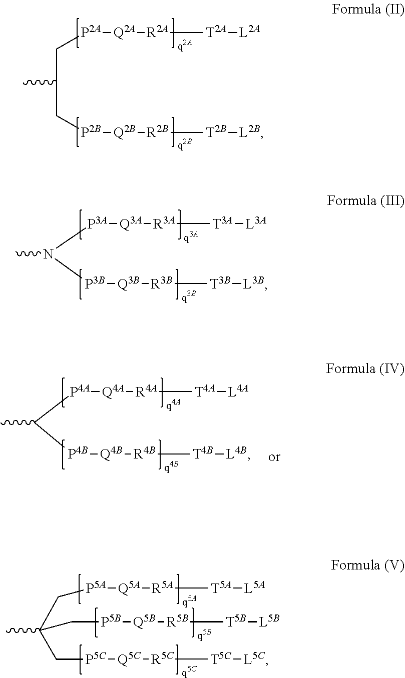

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
P^{2A}, P^{2B}, P^{3A}, P^{3B}, P^{4A}, P^{4B}, P^{5A}, P^{5B}, P^{5C}, P⁷, T^{2A}, T^{2B}, T^{3A}, T^{3B}, T^{4A}, T^{4B}, T^{4A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═C(R′), C≡C or C(O);
T^{B} and T^{B′} are each independently for each occurrence absent, CO, NH, O, S, OC(O), OC(O) O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl) cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a})—NH—, CO, CH═N—O,

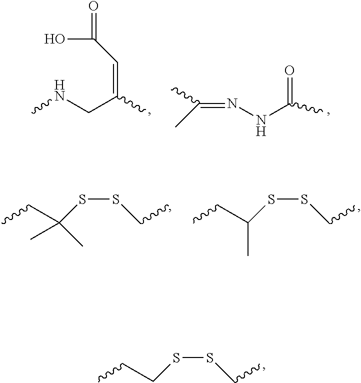

or heterocyclyl;
L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence O or S;
p represents independently for each occurrence 0-20.

As discussed above, because the ligand can be conjugated to the effector molecules (e.g., bis siRNA) or the sciRNA (or bis-sciRNA) agent via a linker or carrier, and because the linker or carrier can contain a branched linker, the effector molecules (e.g., bis siRNA) or the sciRNA (or bis-sciRNA) agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In other embodiment, the branchpoint is glycerol or glycerol derivative.

In certain embodiments, the ASGPR ligand conjugated to the multi-targeted molecule (e.g., the effector molecules such as bis siRNA or the sciRNA (or bis-sciRNA)) is one or more GalNAc derivatives attached through a bivalent or trivalent branched linker.

In certain embodiments, the multi-targeted molecule (e.g., the effector molecules such as bis siRNA or the sciRNA (or bis-sciRNA) agent) comprises a ligand of structure:

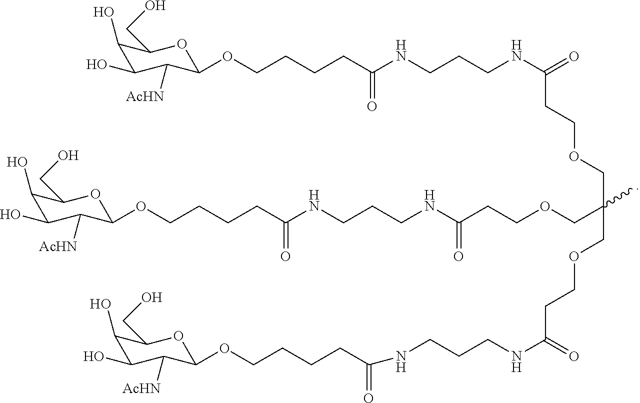 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the multi-targeted molecule (e.g., the effector molecules such as bis siRNA or the sciRNA (or bis-sciRNA) agent) comprises a ligand of structure:

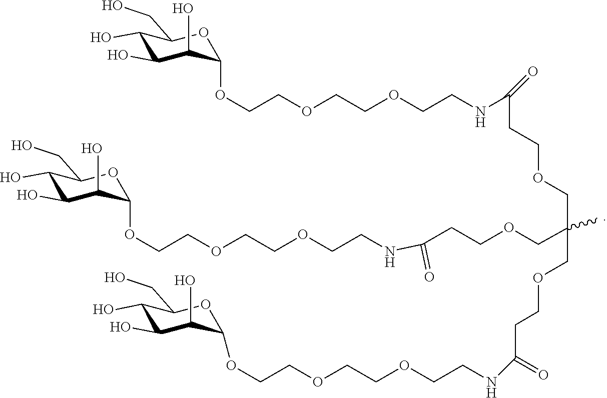

In certain embodiments, the multi-targeted molecule (e.g., the effector molecules such as bis siRNA or the sciRNA (or bis-sciRNA) agent) comprises a ligand of structure:

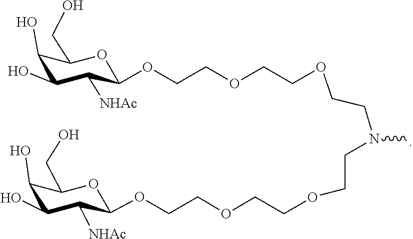

In certain embodiments, the multi-targeted molecule (e.g., the effector molecules such as bis siRNA or the sciRNA (or bis-sciRNA) agent) comprises a ligand of structure:

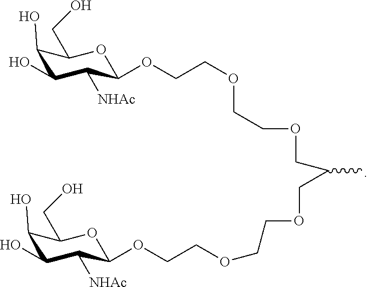

In certain embodiments, the multi-targeted molecule (e.g., the effector molecules such as bis siRNA or the sciRNA (or bis-sciRNA) agent) comprises a ligand of structure:

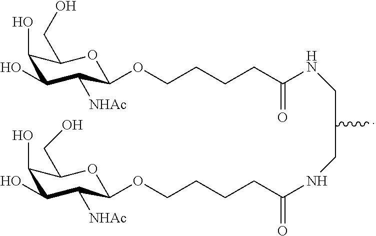

In certain embodiments, the multi-targeted molecule (e.g., the effector molecules such as bis siRNA or the sciRNA (or bis-sciRNA) agent) comprises a ligand of structure:

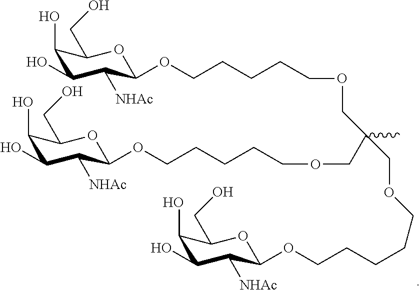

In certain embodiments, the multi-targeted molecule (e.g., the effector molecules such as bis siRNA or the sciRNA (or bis-sciRNA) agent) comprises a ligand of structure:

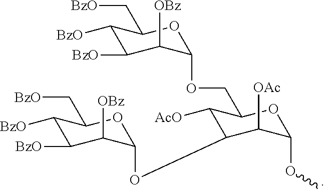

In certain embodiments, the multi-targeted molecule (e.g., the effector molecules such as bis siRNA or the sciRNA (or bis-sciRNA) agent) comprises a ligand of structure:

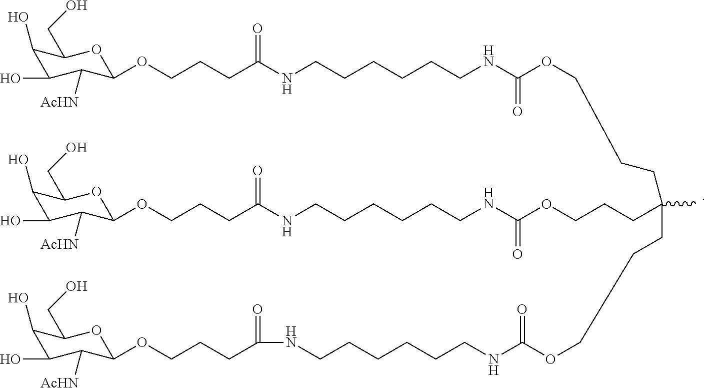 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

In certain embodiments, the multi-targeted molecule (e.g., the effector molecules such as bis siRNA or the sciRNA (or bis-sciRNA) agent) comprises a ligand of structure:

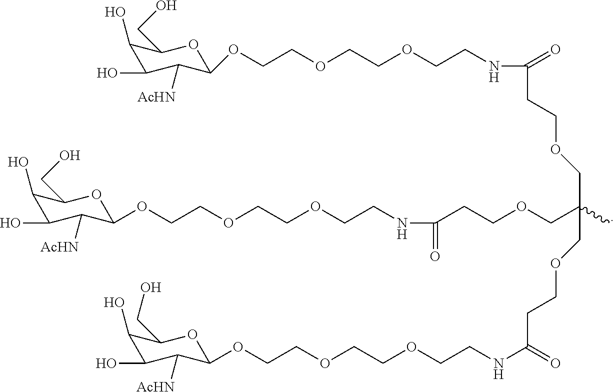 [SMILES: CC(=O)NC1C(OCCOCCOCCNC(=O)CCOCC(C)(COCCC(=O)NCCOCCOCCOC2OC(CO)C(O)C(O)C2NC(C)=O)COCCC(=O)NCCOCCOCCOC2OC(CO)C(O)C(O)C2NC(C)=O)OC(CO)C(O)C1O]

In certain embodiments, the multi-targeted molecule (e.g., the effector molecules such as bis siRNA or the sciRNA (or bis-sciRNA) agent) comprises a ligand of structure:

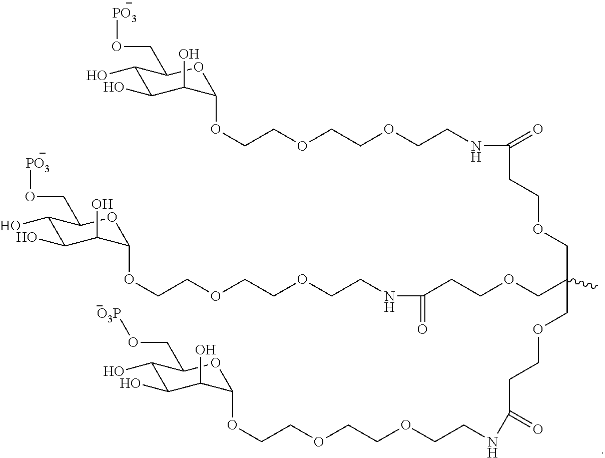

In certain embodiments, the multi-targeted molecule (e.g., the effector molecules such as bis siRNA or the sciRNA (or bis-sciRNA) agent) comprises a ligand of structure:

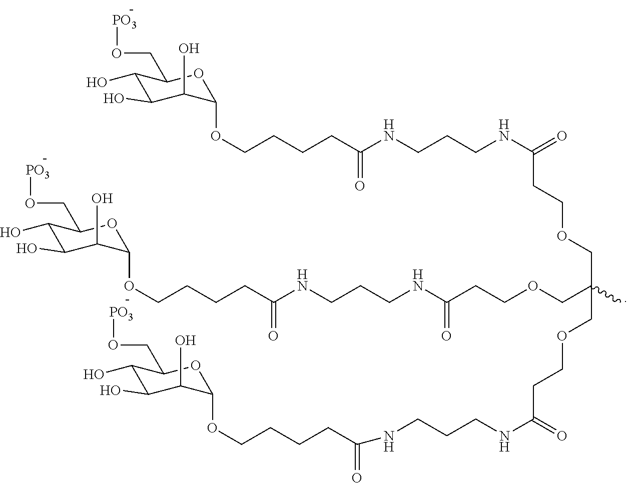

In certain embodiments, the multi-targeted molecule (e.g., the effector molecules such as bis siRNA or the sciRNA (or bis-sciRNA) agent) comprises a monomer of structure:

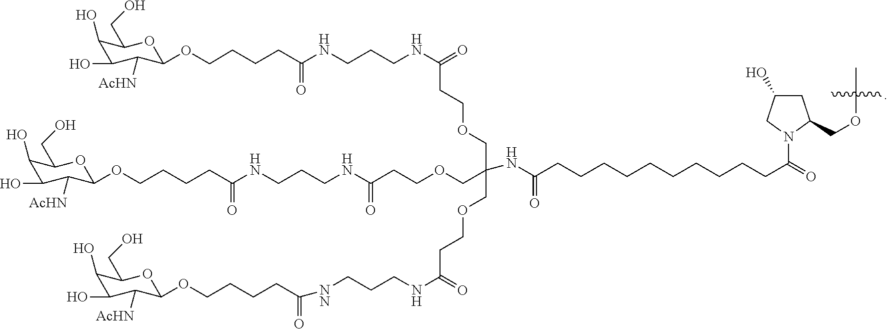

In certain embodiments, the multi-targeted molecule (e.g., the effector molecules such as bis siRNA or the sciRNA (or bis-sciRNA) agent comprises a ligand of structure:

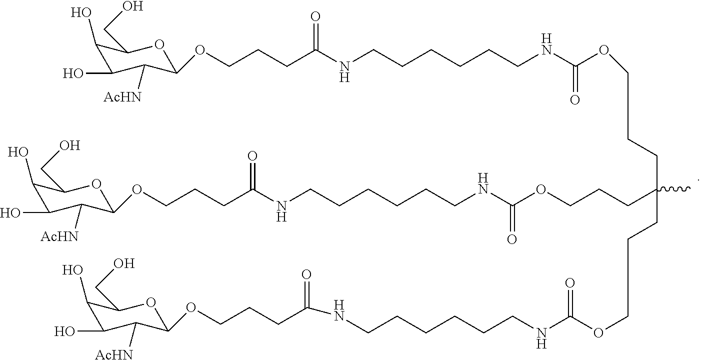 [SMILES: CC(=O)NC1C(OCCCC(=O)NCCCCCCNC(=O)OCCCC(C)(CCCOC(=O)NCCCCCCNC(=O)CCCOC2OC(CO)C(O)C(O)C2NC(C)=O)CCCOC(=O)NCCCCCCNC(=O)CCCOC2OC(CO)C(O)C(O)C2NC(C)=O)OC(CO)C(O)C1O]

### Exemplary Ligand Monomers

In certain embodiments, the multi-targeted molecule (e.g., the effector molecules such as bis siRNA or the sciRNA (or bis-sciRNA) agent) comprises a monomer of structure:

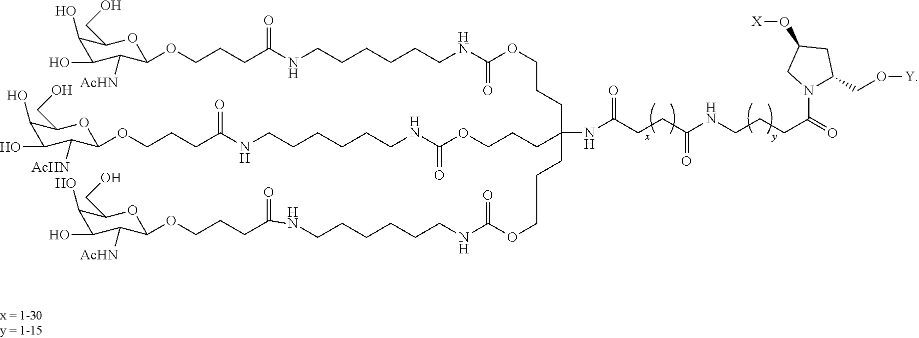

In certain embodiments, the multi-targeted molecule (e.g., the effector molecules such as bis siRNA or the sciRNA (or bis-sciRNA) agent) comprises a monomer of structure:

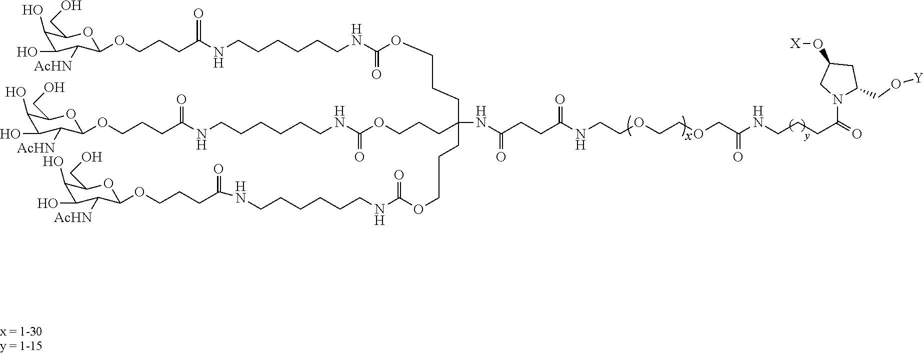

In certain embodiments, the multi-targeted molecule (e.g., the effector molecules such as bis siRNA or the sciRNA (or bis-sciRNA) agent) comprises a monomer of structure:

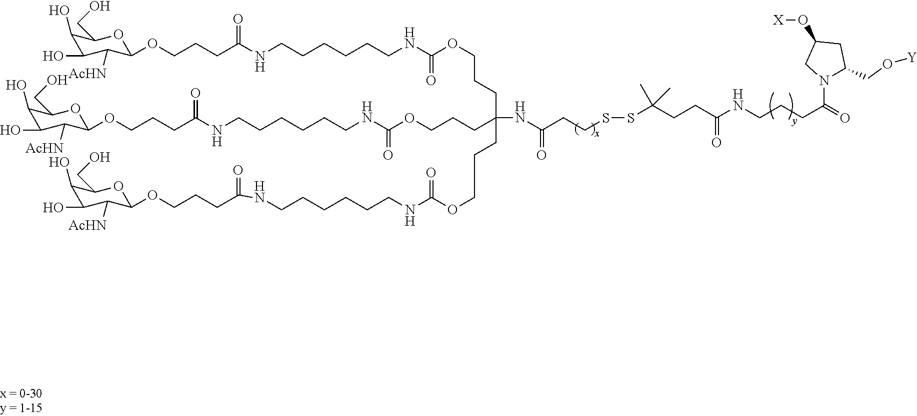

In certain embodiments, the multi-targeted molecule (e.g., the effector molecules such as bis siRNA or the sciRNA (or bis-sciRNA) agent) comprises a monomer of structure:

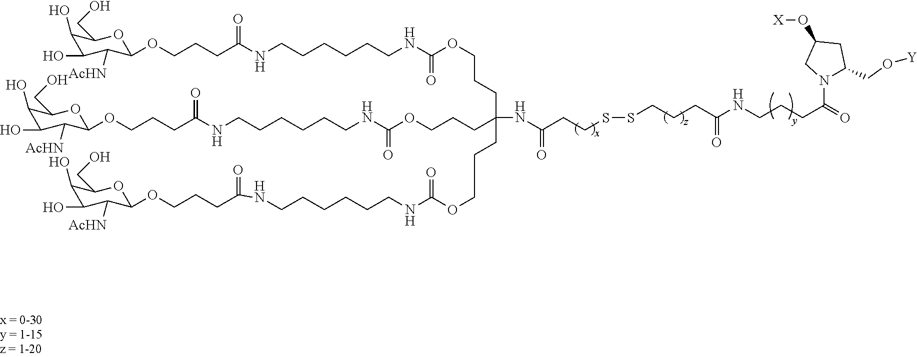

In certain embodiments, the multi-targeted molecule (e.g., the effector molecules such as bis siRNA or the sciRNA (or bis-sciRNA) agent) comprises a monomer of structure:

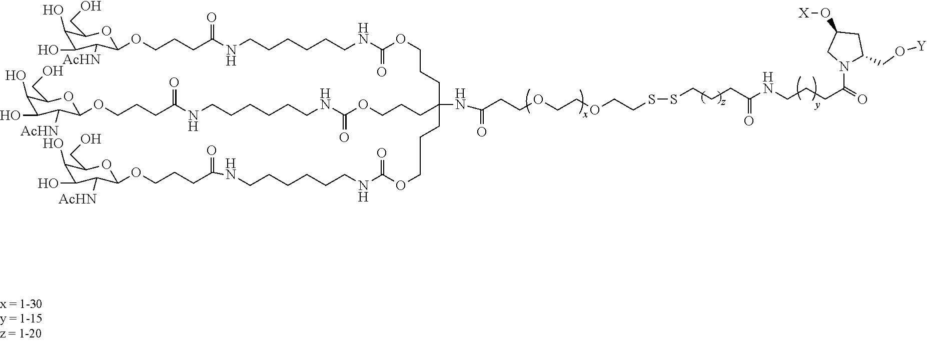

In certain embodiments, the multi-targeted molecule (e.g., the effector molecules such as bis siRNA or the sciRNA (or bis-sciRNA) agent) comprises a monomer of structure:

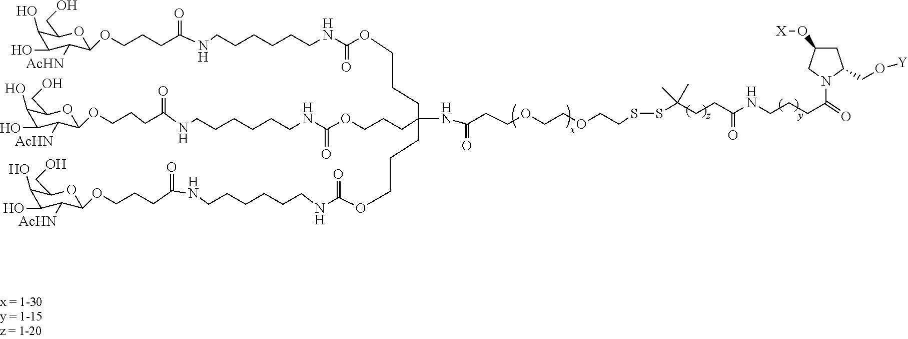

In certain embodiments, the multi-targeted molecule (e.g., the effector molecules such as bis siRNA or the sciRNA (or bis-sciRNA) agent) comprises a ligand of structure:

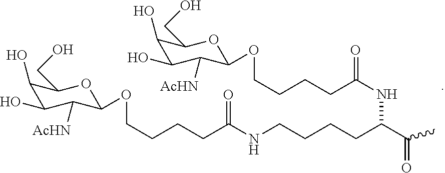

In certain embodiments, the multi-targeted molecule (e.g., the effector molecules such as bis siRNA or the sciRNA (or bis-sciRNA) agent) comprises a ligand of structure:

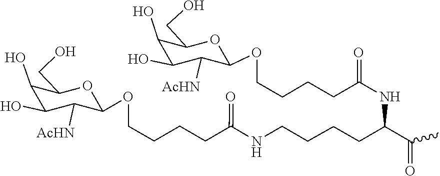

In certain embodiments, the multi-targeted molecule (e.g., the effector molecules such as bis siRNA or the sciRNA (or bis-sciRNA) agent) comprises a ligand of structure:

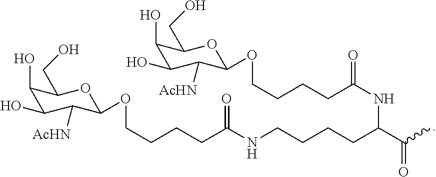

In certain embodiments, the multi-targeted molecule (e.g., the effector molecules such as bis siRNA or the sciRNA (or bis-sciRNA) agent) comprises a ligand of structure:

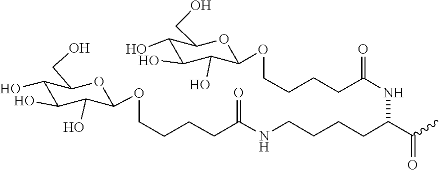

In certain embodiments, the multi-targeted molecule (e.g., the effector molecules such as bis siRNA or the sciRNA (or bis-sciRNA) agent) comprises a ligand of structure:

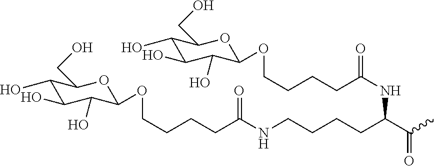

In certain embodiments, the multi-targeted molecule (e.g., the effector molecules such as bis siRNA or the sciRNA (or bis-sciRNA) agent) comprises a ligand of structure:

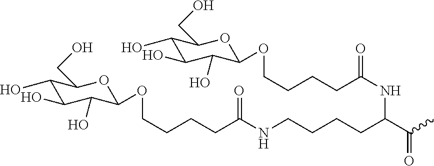

In certain embodiments, the multi-targeted molecule (e.g., the effector molecules such as bis siRNA or the sciRNA (or bis-sciRNA) agent) comprises a ligand of structure:

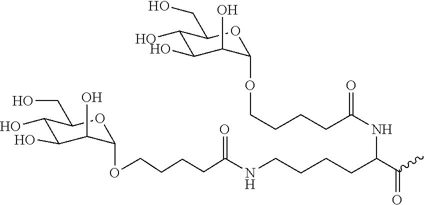

In certain embodiments, the multi-targeted molecule (e.g., the effector molecules such as bis siRNA or the sciRNA (or bis-sciRNA) agent) comprises a ligand of structure:

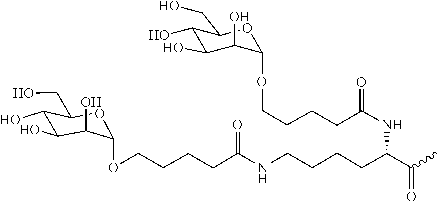

In certain embodiments, the multi-targeted molecule (e.g., the effector molecules such as bis siRNA or the sciRNA (or bis-sciRNA) agent) comprises a ligand of structure:

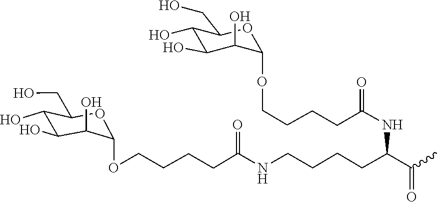

In certain embodiments, the multi-targeted molecule (e.g., the effector molecules such as bis siRNA or the sciRNA (or bis-sciRNA) agent) comprises a monomer of structure:

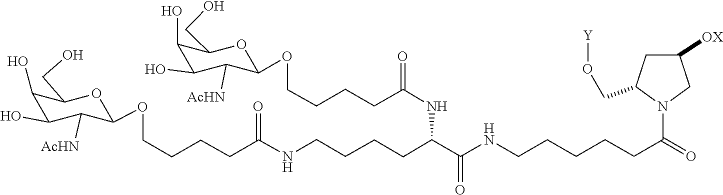

In certain embodiments, the multi-targeted molecule (e.g., the effector molecules such as bis siRNA or the sciRNA (or bis-sciRNA) agent) comprises a monomer of structure:

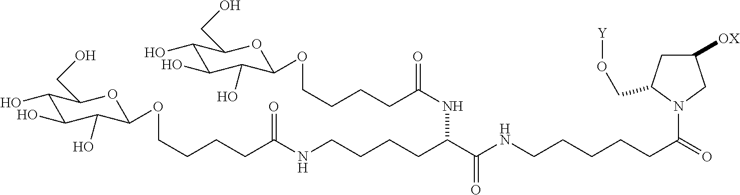

In certain embodiments, the multi-targeted molecule (e.g., the effector molecules such as bis siRNA or the sciRNA (or bis-sciRNA) agent) comprises a monomer of structure:

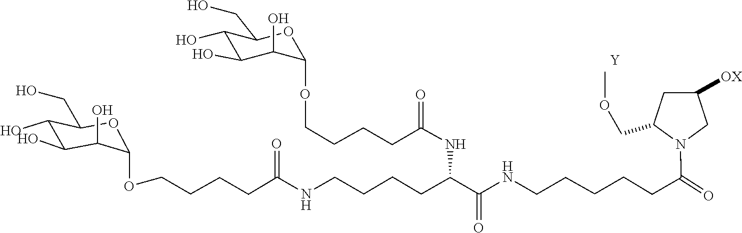

In certain embodiments, the multi-targeted molecule (e.g., the effector molecules such as bis siRNA or the sciRNA (or bis-sciRNA) agent) comprises a monomer of structure:

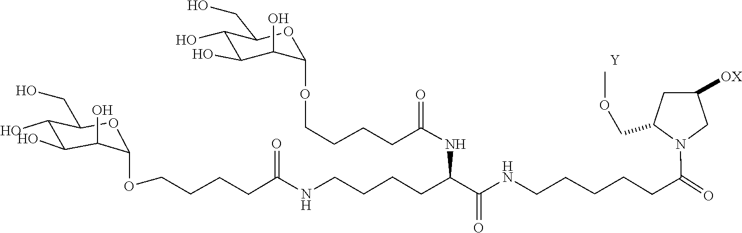

In certain embodiments, the multi-targeted molecule (e.g., the effector molecules such as bis siRNA or the sciRNA (or bis-sciRNA) agent) comprises a monomer of structure:

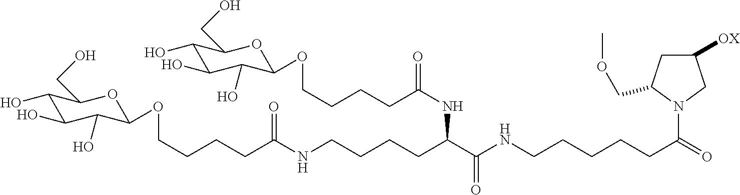

In certain embodiments, the multi-targeted molecule (e.g., the effector molecules such as bis siRNA or the sciRNA (or bis-sciRNA) agent) comprises a monomer of structure:

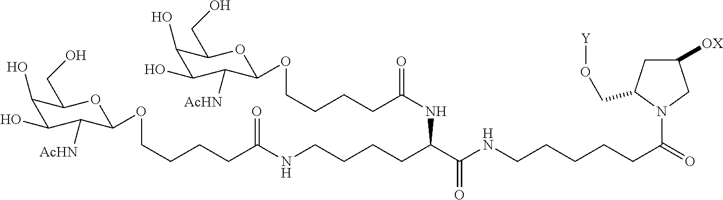

In some embodiments, both L^{2A} and L^{2B} are the same. In some embodiments, both L^{2A} and L^{2B} are different.

In some embodiments, both L^{3A} and L^{3B} are the same. In some embodiments, both L^{3A} and L^{3B} are different.

In some embodiments, both L^{4A} and L^{4B} are the same. In some embodiments, both L^{4A} and L^{4B} are different.

In some embodiments, all of L^{5A}, L^{5B} and L^{5C} are the same. In some embodiments, two of L^{5A}, L^{5B} and L^{5C} are the same. In some embodiments, L^{5A} and L^{5B} are the same. In some embodiments, L^{5A} and L^{5C} are the same. In some embodiments, L^{5B} and L^{5C} are the same.

In certain embodiments, the multi-targeted molecule (e.g., the effector molecules such as bis siRNA or the sciRNA (or bis-sciRNA) agent) comprises a monomer of structure:

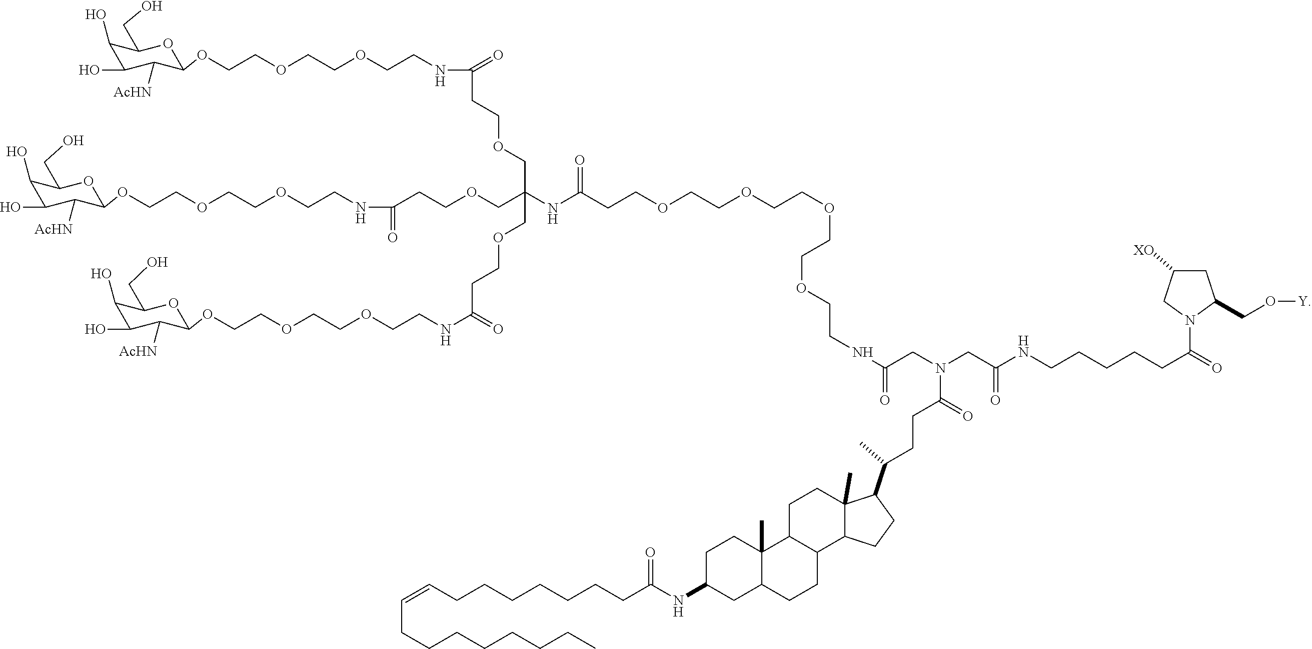

In certain embodiments, the multi-targeted molecule (e.g., the effector molecules such as bis siRNA or the sciRNA (or bis-sciRNA) agent) comprises a monomer of structure:

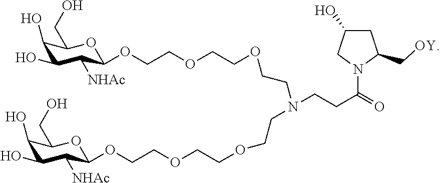

In certain embodiments, the multi-targeted molecule (e.g., the effector molecules such as bis siRNA or the sciRNA (or bis-sciRNA) agent) comprises a monomer of structure:

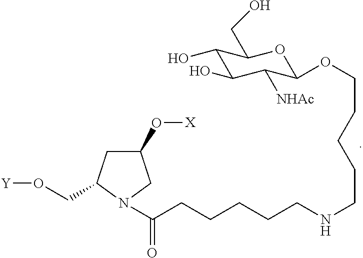

In certain embodiments, the multi-targeted molecule (e.g., the effector molecules such as bis siRNA or the sciRNA (or bis-sciRNA) agent) comprises a monomer of structure:

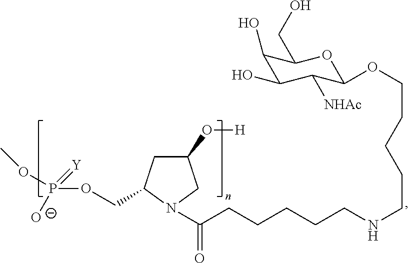

wherein Y is O or S, and n is 1-6.

In certain embodiments, the multi-targeted molecule (e.g., the effector molecules such as bis siRNA or the sciRNA (or bis-sciRNA) agent) comprises a monomer of structure:

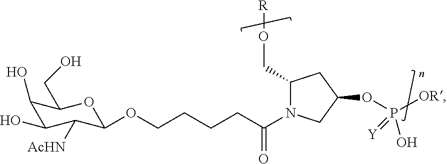

wherein Y is O or S, n is 1-6, R is hydrogen or nucleic acid, and R′ is nucleic acid.

In certain embodiments, the multi-targeted molecule (e.g., the effector molecules such as bis siRNA or the sciRNA (or bis-sciRNA) agent) comprises a monomer of structure:

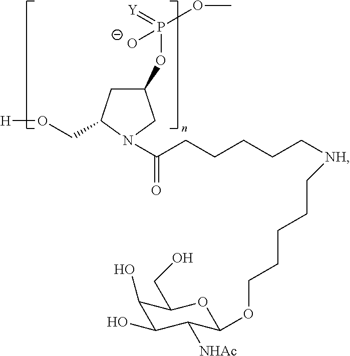

wherein Y is O or S, and n is 1-6.

In certain embodiments, the multi-targeted molecule (e.g., the effector molecules such as bis siRNA or the sciRNA (or bis-sciRNA) agent) comprises a monomer of structure:

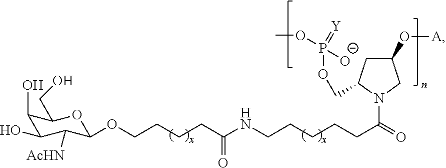

wherein Y is O or S, n is 2-6, x is 1-6, and A is H or a phosphate linkage.

In certain embodiments, the multi-targeted molecule (e.g., the effector molecules such as bis siRNA or the sciRNA (or bis-sciRNA) agent) comprises at least 1, 2, 3 or 4 monomer of structure:

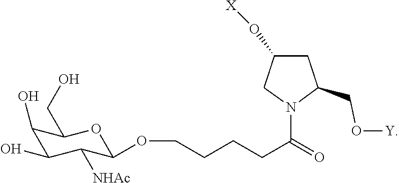

In certain embodiments, the multi-targeted molecule (e.g., the effector molecules such as bis siRNA or the sciRNA (or bis-sciRNA) agent) comprises a monomer of structure:

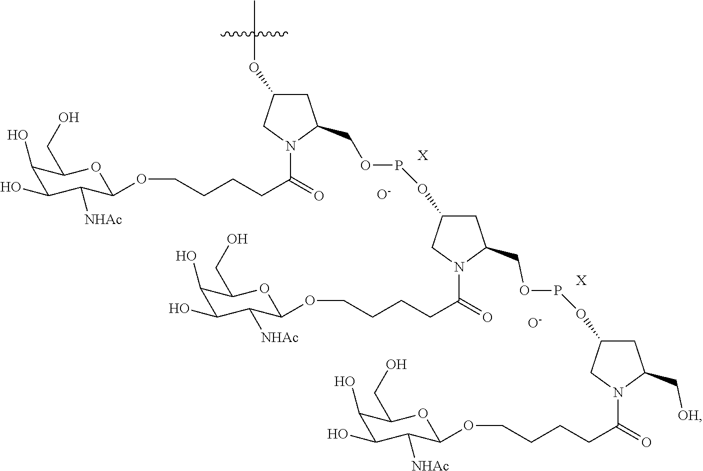

wherein X is O or S.

In certain embodiments, the multi-targeted molecule (e.g., the effector molecules such as bis siRNA or the sciRNA (or bis-sciRNA) agent) comprises a monomer of structure:

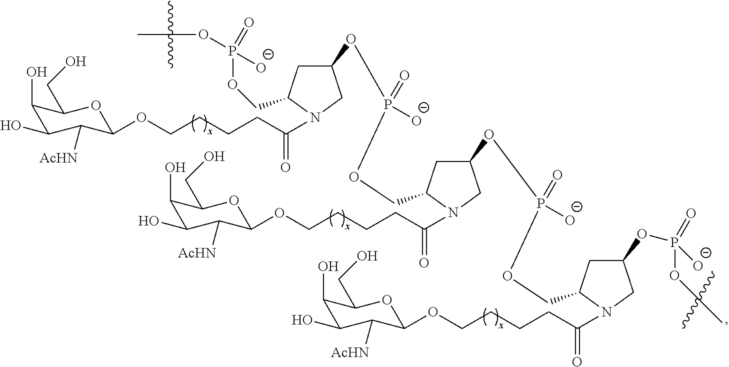

wherein x is 1-12.

In certain embodiments, the multi-targeted molecule (e.g., the effector molecules such as bis siRNA or the sciRNA (or bis-sciRNA) agent) comprises a monomer of structure:

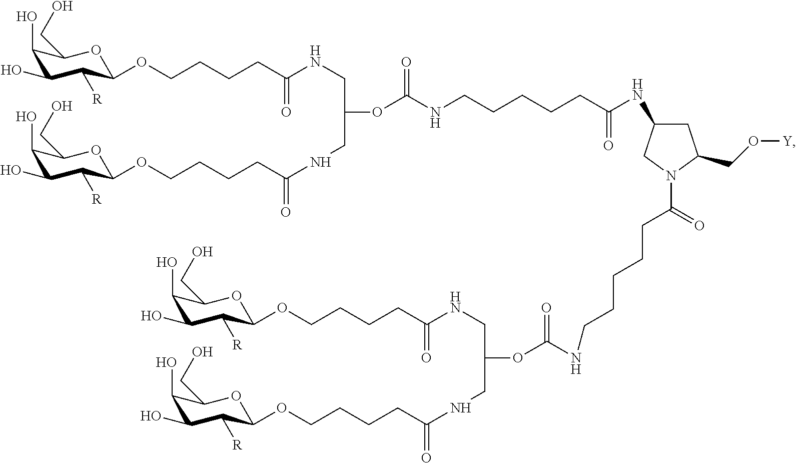

wherein R is OH or NHCOCH₃.

In certain embodiments, the multi-targeted molecule (e.g., the effector molecules such as bis siRNA or the sciRNA (or bis-sciRNA) agent) comprises a monomer of structure:

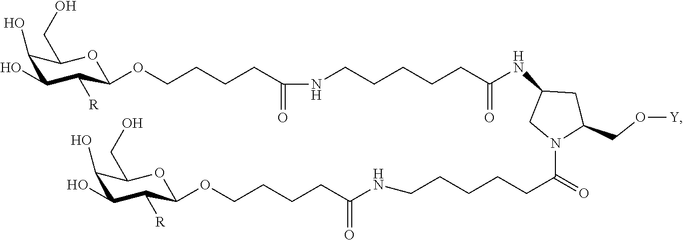

wherein R is OH or NHCOCH₃.

In certain embodiments, the multi-targeted molecule (e.g., the effector molecules such as bis siRNA or the sciRNA (or bis-sciRNA) agent) comprises a monomer of structure:

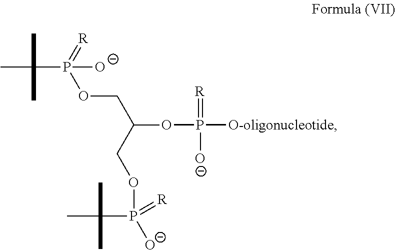

wherein R is O or S.

In certain embodiments, the multi-targeted molecule (e.g., the effector molecules such as bis siRNA or the sciRNA (or bis-sciRNA) agent) comprises a monomer of structure:

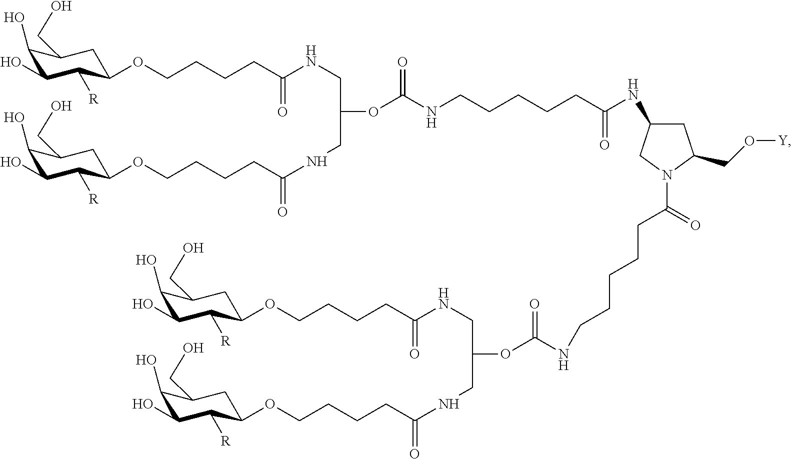

wherein R is OH or NHCOCH₃.

In certain embodiments, the multi-targeted molecule (e.g., the effector molecules such as bis siRNA or the sciRNA (or bis-sciRNA) agent) comprises a monomer of structure:

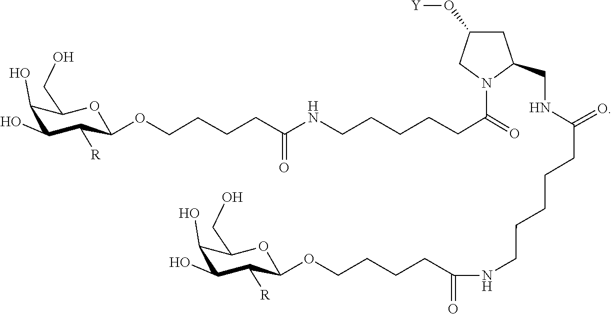

In certain embodiments, the multi-targeted molecule (e.g., the effector molecules such as bis siRNA or the sciRNA (or bis-sciRNA) agent) comprises a monomer of structure:

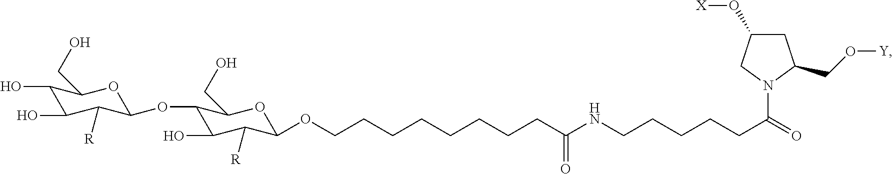

wherein R is OH or NHCOCH₃.

In certain embodiments, the multi-targeted molecule (e.g., the effector molecules such as bis siRNA or the sciRNA (or bis-sciRNA) agent) comprises a monomer of structure:

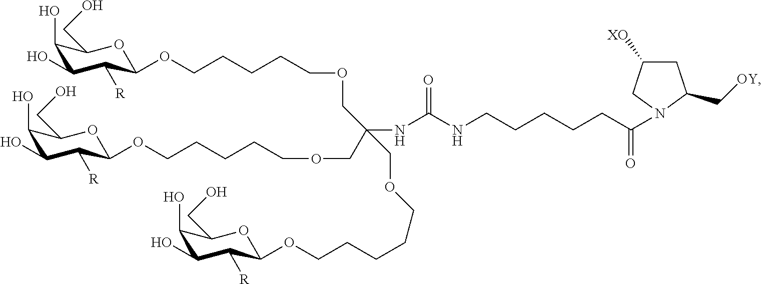

wherein R is OH or NHCOCH₃.

In certain embodiments, the multi-targeted molecule (e.g., the effector molecules such as bis siRNA or the sciRNA (or bis-sciRNA) agent) comprises a monomer of structure:

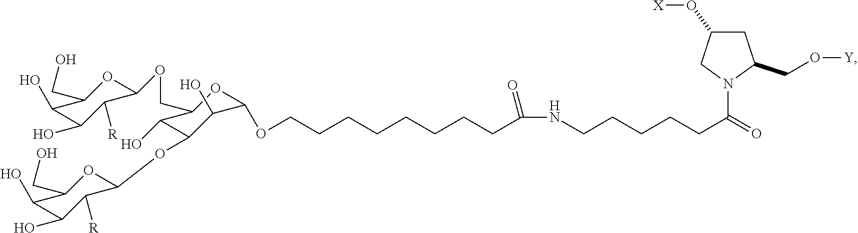

wherein R is OH or NHCOCH₃.

In certain embodiments, the multi-targeted molecule (e.g., the effector molecules such as bis siRNA or the sciRNA (or bis-sciRNA) agent) comprises a monomer of structure:

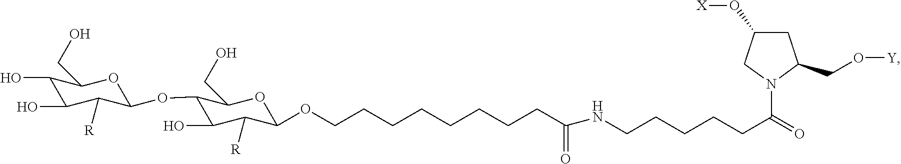

wherein R is OH or NHCOCH₃.

In certain embodiments, the multi-targeted molecule (e.g., the effector molecules such as bis siRNA or the sciRNA (or bis-sciRNA) agent) comprises a monomer of structure:

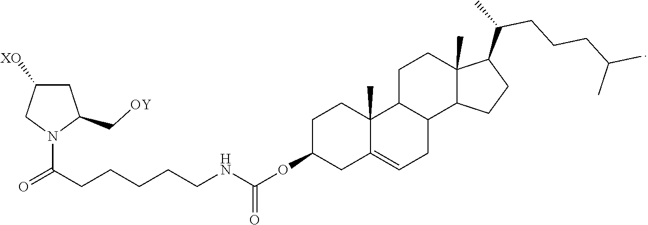

In the above described monomers, X and Y are each independently for each occurrence H, a protecting group, a phosphate group, a phosphodiester group, an activated phosphate group, an activated phosphite group, a phosphoramidite, a solid support, —P(Z′)(Z″)O-nucleoside, —P(Z′)(Z″)O-oligonucleotide, a lipid, a PEG, a steroid, a polymer, a nucleotide, a nucleoside, or an oligonucleotide; and Z′ and Z″ are each independently for each occurrence O or S.

In certain embodiments, the multi-targeted molecule (e.g., the effector molecules such as bis siRNA or the sciRNA (or bis-sciRNA) agent) is conjugated with a ligand of structure:

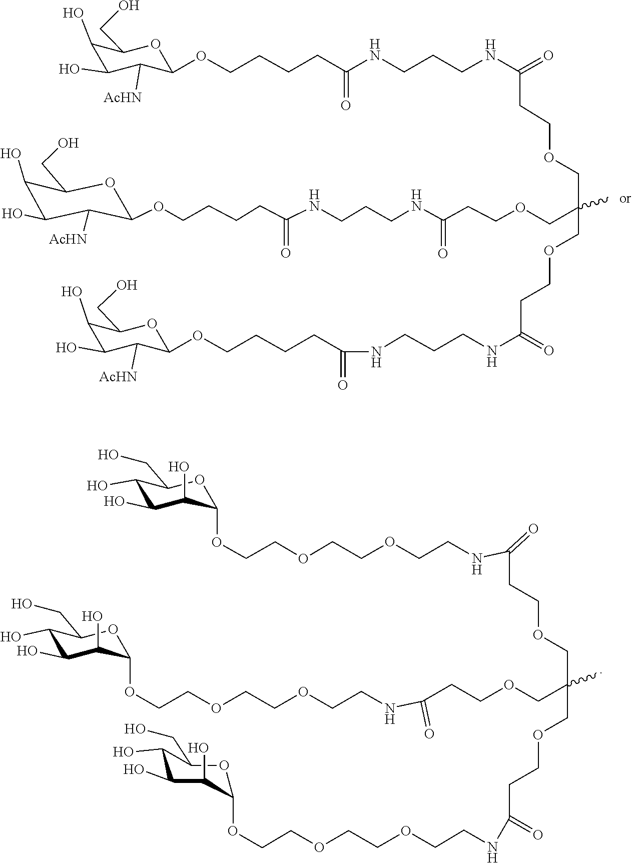

In certain embodiments, the multi-targeted molecule (e.g., the effector molecules such as bis siRNA or the sciRNA (or bis-sciRNA) agent) comprises a ligand of structure:

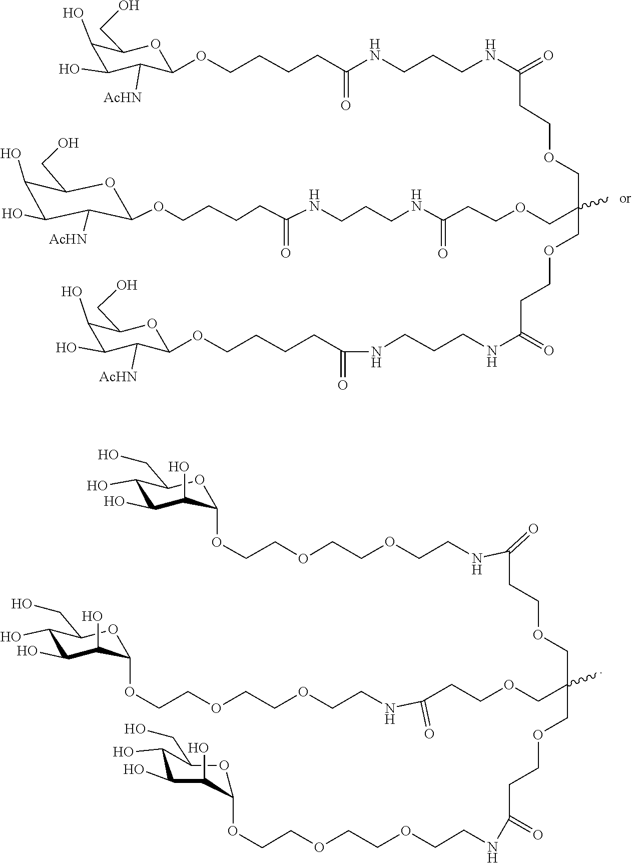

In certain embodiments, the multi-targeted molecule (e.g., the effector molecules such as bis siRNA or the sciRNA (or bis-sciRNA) agent) comprises a monomer of structure:

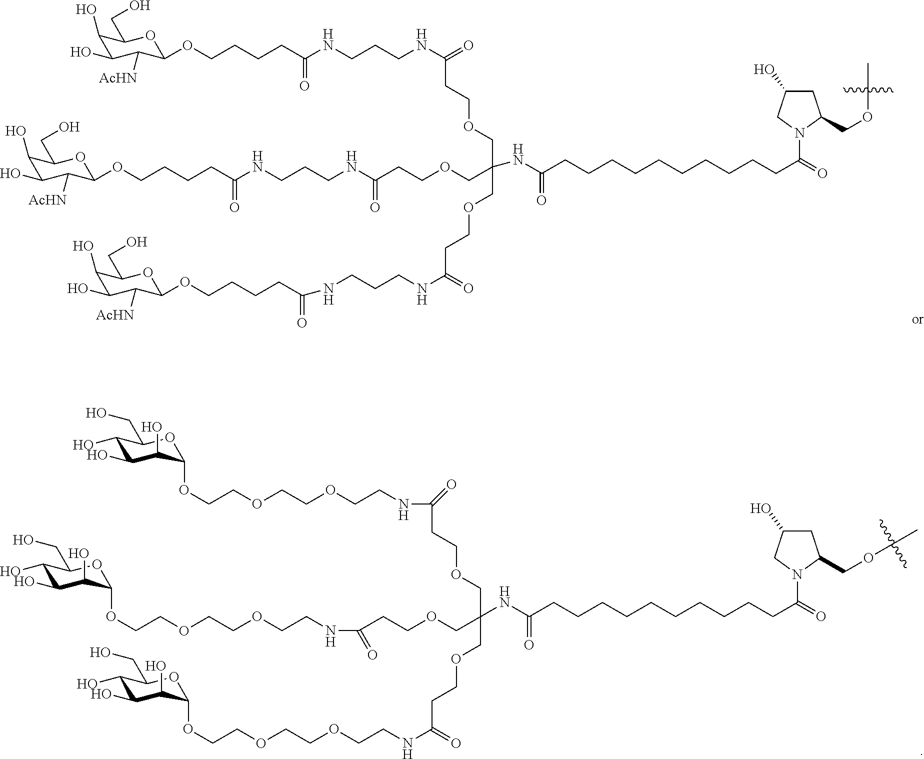

Synthesis of above described ligands and monomers is described, for example, in U.S. Pat. No. 8,106,022, content of which is incorporated herein by reference in its entirety.

In certain embodiments, at least one of the ligands conjugated to the multi-targeted molecule (e.g., the effector molecules such as bis siRNA or the sciRNA (or bis-sciRNA)) is a lipophilic moiety.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Alternatively, the hydrophobicity of the multi-targeted molecule (e.g., the effector molecules such as bis siRNA or the sciRNA (or bis-sciRNA) agent), conjugated to one or more lipophilic moieties, can be measured by its protein binding characteristics. For instance, the unbound fraction in the plasma protein binding assay of the effector molecule (e.g., bis siRNA) or the sciRNA (or bis-sciRNA) agent can be determined to positively correlate to the relative hydrophobicity of the effector molecule (e.g., bis siRNA) or the sciRNA (or bis-sciRNA) agent, which can positively correlate to the silencing activity of the effector molecule (e.g., bis siRNA) or the sciRNA (or bis-sciRNA) agent.

In one embodiment, the plasma protein binding assay determined is an electrophoretic mobility shift assay (EMSA) using human serum albumin protein. The hydrophobicity of the multi-targeted molecule (e.g., the effector molecules such as bis siRNA or the sciRNA (or bis-sciRNA) agent), measured by fraction of unbound the effector molecules (e.g., bis siRNA) or sciRNA (or bis-sciRNA) in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of the effector molecule (e.g., bis siRNA) or sciRNA (or bis-sciRNA).

Accordingly, conjugating the lipophilic moieties to the internal position(s) of the multi-targeted molecule (e.g., the effector molecules such as bis siRNA or the sciRNA (or bis-sciRNA) agent) provides optimal hydrophobicity for the enhanced in vivo delivery of the effector molecules (e.g., bis siRNA) or sciRNA (or bis-sciRNA).

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₁₈ alkyl or alkenyl). In one embodiment, the lipophilic moiety contains a saturated or unsaturated C₁₆ hydrocarbon chain (e.g., a linear C₁₆ alkyl or alkenyl).

The lipophilic moiety may be attached to the effector molecule (e.g., bis siRNA) or the sciRNA (or bis-sciRNA) agent by any method known in the art, including via a functional grouping already present in the lipophilic moiety or introduced into the effector molecule (e.g., bis siRNA) or the sciRNA (or bis-sciRNA) agent, such as a hydroxy group (e.g., —CO—CH₂—OH). The functional groups already present in the lipophilic moiety or introduced into the effector molecule (e.g., bis siRNA) or the sciRNA (or bis-sciRNA) agent include, but are not limited to, hydroxyl, amine, carboxylic acid, sulfonate, phosphate, thiol, azide, and alkyne.

Conjugation of the effector molecule (e.g., bis siRNA) or the sciRNA (or bis-sciRNA) agent and the lipophilic moiety may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the lipophilic moiety is conjugated to the effector molecule (e.g., bis siRNA) or the sciRNA (or bis-sciRNA) agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are herey incorporated by reference in their entirety. Naproxen has the chemical name (S)-6-Methoxy-α-methyl-2-naphthaleneacetic acid and the structure is

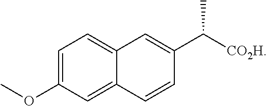 [SMILES: COc1ccc2cc([C@H](C)C(=O)O)ccc2c1]

In certain embodiments, the ligand is ibuprofen or a structural derivative of ibuprofen. Procedures for the synthesis of ibuprofen can be found in U.S. Pat. No. 3,228,831, which are herey incorporated by reference in their entirety. The structure of ibuprofen is

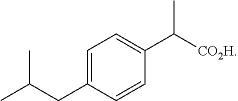

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl)lithocholic acid, O3-(oleoyl)cholenic acid, ibuprofen, naproxen, dimethoxytrityl, or phenoxazine.

In some embodiments, the lipophilic moiety is a C₆-C₃₀ acid (e.g., hexanoic acid, heptanoic acid, octanoic acid, nonanoic acid, decanoic acid, undecanoic acid, dodcanoic acid, tridecanoic acid, tetradecanoic acid, pentadecanoic acid, hexadecanoic acid, heptadecanoic acid, octadecanoic acid, oleic acid, linoleic acid, arachidonic acid, cis-4,7,10,13,16,19-docosahexanoic acid, vitamin A, vitamin E, cholesterol etc.) or a C₆-C₃₀ alcohol (e.g., hexanol, heptanol, octanol, nonanol, decanol, undecanol, dodcanol, tridecanol, tetradecanol, pentadecanol, hexadecanol, heptadecanol, octadecanol, oleyl alcohol, linoleyl alcohol, arachidonic alcohol, cis-4,7,10,13,16,19-docosahexanol, retinol, vitamin E, cholesterol etc.). In one example, the lipohilic moiety is docosahexaenoic acid.

In certain embodiments, more than one lipophilic moieties can be incorporated into the multi-targeted molecule (e.g., the effector molecules such as bis siRNA or the sciRNA (or bis-sciRNA) agent), particularly when the lipophilic moiety has a low lipophilicity or hydrophobicity. In one embodiment, two or more lipophilic moieties are incorporated into the same strand of the effector molecule (e.g., bis siRNA) or the sciRNA (or bis-sciRNA) agent. In one embodiment, each strand of the effector molecule (e.g., bis siRNA) or the sciRNA (or bis-sciRNA) agent has one or more lipophilic moieties incorporated. In one embodiment, two or more lipophilic moieties are incorporated into the same position (i.e., the same nucleobase, same sugar moiety, or same internucleosidic linkage) of the effector molecule (e.g., bis siRNA) or the sciRNA (or bis-sciRNA) agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the effector molecule (e.g., bis siRNA) or the sciRNA (or bis-sciRNA) agent via a direct attachment to the ribosugar of the sciRNA (or bis-sciRNA) agent. Alternatively, the lipophilic moiety may be conjugated to the effector molecule (e.g., bis siRNA) or the sciRNA (or bis-sciRNA) agent via a linker or a carrier.

In certain embodiments, the lipophilic moiety may be conjugated to the effector molecule (e.g., bis siRNA) or the sciRNA (or bis-sciRNA) agent via one or more linkers (tethers).

In one embodiment, the lipophilic moiety is conjugated to the effector molecule (e.g., bis siRNA) or the sciRNA (or bis-sciRNA) agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### Definitions

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, the term “target nucleic acid” refers to any nucleic acid molecule the expression or activity of which is capable of being modulated by an siRNA compound. Target nucleic acids include, but are not limited to, RNA (including, but not limited to pre-mRNA and mRNA or portions thereof) transcribed from DNA encoding a target protein, and also cDNA derived from such RNA, and miRNA. For example, the target nucleic acid can be a cellular gene (or mRNA transcribed from the gene) whose expression is associated with a particular disorder or disease state. In some embodiments, a target nucleic acid can be a nucleic acid molecule from an infectious agent.

The term “target sequence” or “target RNA sequence” refers to a contiguous portion of the nucleotide sequence of a RNA molecule formed during the transcription of a target gene or other regulatory element, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T”, and “U” each generally stand for a nucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, thymidine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

As used herein, the term “iRNA” refers to an agent that mediates the targeted cleavage of an RNA transcript. These agents associate with a cytoplasmic multi-protein complex known as RNAi-induced silencing complex (RISC). Agents that are effective in inducing RNA interference are also referred to as siRNA, RNAi agent, or iRNA agent, herein. Thus, these terms can be used interchangeably herein. As used herein, the term iRNA includes microRNAs and pre-microRNAs. Moreover, the “compound” or “compounds” as used herein, also refers to the iRNA agent, and can be used interchangeably with the iRNA agent.

The iRNA agent should include a region of sufficient homology to the target gene, and be of sufficient length in terms of nucleotides, such that the iRNA agent, or a fragment thereof, can mediate downregulation of the target gene. (For ease of exposition the term nucleotide or ribonucleotide is sometimes used herein in reference to one or more monomeric subunits of an iRNA agent. It will be understood herein that the usage of the term “ribonucleotide” or “nucleotide”, herein can, in the case of a modified RNA or nucleotide surrogate, also refer to a modified nucleotide, or surrogate replacement moiety at one or more positions.) Thus, the iRNA agent is or includes a region which is at least partially, and in some embodiments fully, complementary to the target RNA. It is not necessary that there be perfect complementarity between the iRNA agent and the target, but the correspondence must be sufficient to enable the iRNA agent, or a cleavage product thereof, to direct sequence specific silencing, e.g., by RNAi cleavage of the target RNA, e.g., mRNA. Complementarity, or degree of homology with the target strand, is most critical in the antisense strand. While perfect complementarity, particularly in the antisense strand, is often desired some embodiments can include, particularly in the antisense strand, one or more, or for example, 6, 5, 4, 3, 2, or fewer mismatches (with respect to the target RNA). The sense strand need only be sufficiently complementary with the antisense strand to maintain the over all double stranded character of the molecule.

iRNA agents include: molecules that are long enough to trigger the interferon response (which can be cleaved by Dicer (Bernstein et al. 2001. Nature, 409:363-366) and enter a RISC (RNAi-induced silencing complex)); and, molecules which are sufficiently short that they do not trigger the interferon response (which molecules can also be cleaved by Dicer and/or enter a RISC), e.g., molecules which are of a size which allows entry into a RISC, e.g., molecules which resemble Dicer-cleavage products. Molecules that are short enough that they do not trigger an interferon response are termed siRNA agents or shorter iRNA agents herein. “siRNA agent or shorter iRNA agent” as used herein, refers to an iRNA agent, e.g., a double stranded RNA agent or single strand agent, that is sufficiently short that it does not induce a deleterious interferon response in a human cell, e.g., it has a duplexed region of less than 60, 50, 40, or 30 nucleotide pairs. The siRNA agent, or a cleavage product thereof, can down regulate a target gene, e.g., by inducing RNAi with respect to a target RNA, wherein the target may comprise an endogenous or pathogen target RNA.

A “single strand iRNA agent” as used herein, is an iRNA agent which is made up of a single molecule. It may include a duplexed region, formed by intra-strand pairing, e.g., it may be, or include, a hairpin or pan-handle structure. Single strand iRNA agents may be antisense with regard to the target molecule. A single strand iRNA agent may be sufficiently long that it can enter the RISC and participate in RISC mediated cleavage of a target mRNA. A single strand iRNA agent is at least 14, and in other embodiments at least 15, 20, 25, 29, 35, 40, or 50 nucleotides in length. In certain embodiments, it is less than 200, 100, or 60 nucleotides in length.

A loop refers to a region of an iRNA strand that is unpaired with the opposing nucleotide in the duplex when a section of the iRNA strand forms base pairs with another strand or with another section of the same strand.

Hairpin iRNA agents will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region, in some embodiments at the 3′, and in certain embodiments on the antisense side of the hairpin. In some embodiments, the overhangs are 2-3 nucleotides in length.

A “double stranded (ds) iRNA agent” as used herein, is an iRNA agent which includes more than one, and in some cases two, strands in which interchain hybridization can form a region of duplex structure.

As used herein, the terms “siRNA activity” and “RNAi activity” refer to gene silencing by an siRNA.

In one embodiment, an RNAi agent of the disclosure includes one or more single stranded RNAi molecules (e.g., effector molecules), each of which interacts with a target RNA sequence, e.g., a target mRNA sequence, to direct the cleavage of the target RNA. Without wishing to be bound by theory, it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15: 485). Dicer, a ribonuclease-III-like enzyme, processes these dsRNA into 19-23 base pair short interfering RNAs with characteristic two base 3′ overhangs (Bernstein, et al., (2001) Nature 409: 363). These siRNAs are then incorporated into an RNA-induced silencing complex (RISC) where one or more helicases unwind the siRNA duplex, enabling the complementary antisense strand to guide target recognition (Nykanen, et al., (2001) Cell 107: 309). Upon binding to the appropriate target mRNA, one or more endonucleases within the RISC cleave the target to induce silencing (Elbashir, et al., (2001) Genes Dev. 15: 188). Thus, in one aspect the disclosure relates to a single stranded RNA (ssRNA) (the antisense strand of a siRNA duplex) generated within a cell and which promotes the formation of a RISC complex to effect silencing of the target gene. Accordingly, the term “siRNA” is also used herein to refer to an RNAi as described above. In certain embodiments, one or more dsRNAs of the multi-targeted molecules of the instant disclosure are individually siRNAs (e.g., in the absence of or post-cleavage of a linker that joins together individual dsRNAs of the multi-targeted molecules of the instant disclosure).

In another embodiment, an RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150: 883-894. In certain embodiments, one or more dsRNAs of the multi-targeted molecules of the instant disclosure are individually single-stranded RNAs (e.g., in the absence of or post-cleavage of a linker that joins together individual dsRNAs of the multi-targeted molecules of the instant disclosure).

The term “sciRNA” as used herein, refers to a small circular iRNA agent, that has at least one strand (e.g, a sense strand) that has a circular or substantially circular structure, whereas the other strand (e.g., an antisense strand) can have a linear structure that is annealed to the strand that has a circular or substantially circular structure. Alternatively, the sciRNA can have a circular or substantially circular antisense strand and a linear sense strand that is annealed to the circular or substantially circular antisense strand. It is also possible both sense strand and antisense strands have a circular or substantially circular structure.

The term “sciRNA,” “bis-sciRNA,” and “multi-targeted bis-sciRNA molecule” can be used interchangeable herein to refer to the sciRNA of the disclosure.

As used herein, “gene silencing” by a RNA interference molecule refers to a decrease in the mRNA level in a cell for a target gene by at least about 5%, at least about 10%, at least about 20%, at least about 30%, at least about 40%, at least about 50%, at least about 60%, at least about 70%, at least about 80%, at least about 90%, at least about 95%, at least about 99% up to and including 100%, and any integer in between of the mRNA level found in the cell without the presence of the miRNA or RNA interference molecule. In one preferred embodiment, the mRNA levels are decreased by at least about 70%, at least about 80%, at least about 90%, at least about 95%, at least about 99%, up to and including 100% and any integer in between 5% and 100%.”

As used herein the term “modulate gene expression” means that expression of the gene, or level of RNA molecule or equivalent RNA molecules encoding one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition.

As used herein, gene expression modulation happens when the expression of the gene, or level of RNA molecule or equivalent RNA molecules encoding one or more proteins or protein subunits is at least 5%, 10%, 20%, 30%, 40%, 50%, 60%, 70%, 80%, 90%, 100%, 2-fold, 3-fold, 4-fold, 5-fold or more different from that observed in the absence of the siRNA. The % and/or fold difference can be calculated relative to the control or the non-control, for example,

$$\% difference = \frac{[ expression with siRNA - expression without siRNA ]}{expression without siRNA}$$

$$or$$

$$\% difference = \frac{[ expression with siRNA - expression without siRNA ]}{expression without siRNA}$$

As used herein, the term “inhibit”, “down-regulate”, or “reduce” in relation to gene expression, means that the expression of the gene, or level of RNA molecules or equivalent RNA molecules encoding one or more proteins or protein subunits, or activity of one or more proteins or protein subunits, is reduced below that observed in the absence of modulator. The gene expression is down-regulated when expression of the gene, or level of RNA molecules or equivalent RNA molecules encoding one or more proteins or protein subunits, or activity of one or more proteins or protein subunits, is reduced at least 10% lower relative to a corresponding non-modulated control, and preferably at least 10%, 20%, 30%, 40%, 50%, 60%, 70%, 80%, 90%, 95%, 98%, 99% or most preferably, 100% (i.e., no gene expression).

As used herein, the term “increase” or “up-regulate” in relation to gene expression means that the expression of the gene, or level of RNA molecules or equivalent RNA molecules encoding one or more proteins or protein subunits, or activity of one or more proteins or protein subunits, is increased above that observed in the absence of modulator. The gene expression is up-regulated when expression of the gene, or level of RNA molecules or equivalent RNA molecules encoding one or more proteins or protein subunits, or activity of one or more proteins or protein subunits, is increased at least 10% relative to a corresponding non-modulated control, and preferably at least 10%, 20%, 30%, 40%, 50%, 60%, 70%, 80%, 90%, 95%, 98%, 100%, 1.1-fold, 1.25-fold, 1.5-fold, 1.75-fold, 2-fold, 3-fold, 4-fold, 5-fold, 10-fold, 50-fold, 100-fold or more.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced” or “reduce” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “off-target” and the phrase “off-target effects” refer to any instance in which an effector molecule against a given target causes an unintended affect by interacting either directly or indirectly with another target sequence, a DNA sequence or a cellular protein or other moiety. For example, an “off-target effect” may occur when there is a simultaneous degradation of other transcripts due to partial homology or complementarity between that other transcript and the sense and/or antisense strand of an siRNA.

As used herein, the term “nucleoside” means a glycosylamine comprising a nucleobase and a sugar. Nucleosides includes, but are not limited to, naturally occurring nucleosides, abasic nucleosides, modified nucleosides, and nucleosides having mimetic bases and/or sugar groups.

As used herein, the term “nucleotide” refers to a glycosylamine comprising a nucleobase and a sugar having a phosphate group covalently linked to the sugar. Nucleotides may be modified with any of a variety of substituents.

As used herein, the term “nucleobase” refers to the base portion of a nucleoside or nucleotide. A nucleobase may comprise any atom or group of atoms capable of hydrogen bonding to a base of another nucleic acid.

As used herein, the term “heterocyclic base moiety” refers to a nucleobase comprising a heterocycle.

As used herein, the term “oligomeric compound” refers to a polymeric structure comprising two or more sub-structures and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antidote compounds. In certain embodiments, oligomeric compounds comprise conjugate groups.

As used herein “oligonucleoside” refers to an oligonucleotide in which the internucleoside linkages do not contain a phosphorus atom.

As used herein, the term “oligonucleotide” refers to an oligomeric compound comprising a plurality of linked nucleosides. In certain embodiment, one or more nucleotides of an oligonucleotide is modified. In certain embodiments, an oligonucleotide comprises ribonucleic acid (RNA) or deoxyribonucleic acid (DNA). In certain embodiments, oligonucleotides are composed of naturally- and/or non-naturally-occurring nucleobases, sugars and covalent internucleoside linkages, and can further include non-nucleic acid conjugates.

As used herein “internucleoside linkage” refers to a covalent linkage between adjacent nucleosides.

As used herein “naturally occurring internucleoside linkage” refers to a 3′ to 5′ phosphodiester linkage.

As used herein the term “detecting siRNA activity” or “measuring siRNA activity” means that a test for detecting or measuring siRNA activity is performed on a particular sample and compared to that of a control sample. Such detection and/or measuring can include values of zero. Thus, if a test for detection of siRNA activity results in a finding of no siRNA activity (siRNA activity of zero), the step of “detecting siRNA activity” has nevertheless been performed.

As used herein the term “control sample” refers to a sample that has not been contacted with a test or reporter oligomer compound.

As used herein, the term “motif” refers to the pattern of unmodified and modified nucleotides in an oligomeric compound.

As used herein, the term “chimeric oligomer” refers to an oligomeric compound, having at least one sugar, nucleobase or internucleoside linkage that is differentially modified as compared to at least on other sugar, nucleobase or internucleoside linkage within the same oligomeric compound. The remainder of the sugars, nucleobases and internucleoside linkages can be independently modified or unmodified, the same or different.

As used herein, the term “chimeric oligonucleotide” refers to an oligonucleotide, having at least one sugar, nucleobase or internucleoside linkage that is differentially modified as compared to at least on other sugar, nucleobase or internucleoside linkage within the same oligonucleotide. The remainder of the sugars, nucleobases and internucleoside linkages can be independently modified or unmodified, the same or different.

As used herein, the term “mixed-backbone oligomeric compound” refers to an oligomeric compound wherein at least one internucleoside linkage of the oligomeric compound is different from at least one other internucleoside linkage of the oligomeric compound.

As used herein, the term “target protein” refers to a protein, the modulation of which is desired.

As used herein, the term “target gene” refers to a gene encoding a target protein.

As used herein, the term “targeting” or “targeted to” refers to the association of antisense strand of an effector molecule of a multi-targeted molecule as disclosed herein (e.g., an siRNA effector molecule) to a particular target nucleic acid molecule or a particular region of nucleotides within a target nucleic acid molecule.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

The multi-targeted molecule (e.g., the effector molecules or the sciRNAs comprise two oligonucleotide strands that are sufficiently complementary to hybridize to form a duplex structure. Generally, the duplex structure is between 15 and 30, more generally between 18 and 25, yet more generally between 19 and 24, and most generally between 19 and 21 base pairs in length. In some embodiments, longer double-stranded iRNAs of between 25 and 30 base pairs in length are preferred. In some embodiments, shorter double-stranded iRNAs of between 10 and 15 base pairs in length are preferred. In another embodiment, the double-stranded iRNA is at least 21 nucleotides long.

In some embodiments, the effector molecule or the sciRNA comprises a sense strand and an antisense strand, wherein the antisense strand has a region of complementarity which is complementary to at least a part of a target sequence, and the duplex region is 14-30 nucleotides in length. Similarly, the region of complementarity to the target sequence is between 14 and 30, more generally between 18 and 25, yet more generally between 19 and 24, and most generally between 19 and 21 nucleotides in length.

The phrase “antisense strand” as used herein, refers to an oligomeric compound that is substantially or 100% complementary to a target sequence of interest. The phrase “antisense strand” includes the antisense region of both oligomeric compounds that are formed from two separate strands, as well as unimolecular oligomeric compounds that are capable of forming hairpin or dumbbell type structures. The terms “antisense strand” and “guide strand” are used interchangeably herein.

The phrase “sense strand” refers to an oligomeric compound that has the same nucleoside sequence, in whole or in part, as a target sequence such as a messenger RNA or a sequence of DNA. The terms “sense strand” and “passenger strand” are used interchangeably herein.

By “specifically hybridizable” and “complementary” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 5 nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize through nucleobase complementarity and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

In certain embodiments, an oligomeric compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are oligomeric compounds (e.g., multi-targeted molecules, including effector molecules comprising siRNAs and the like) that may comprise up to about 20% nucleotides that are mismatched (i.e., are not nucleobase complementary to the corresponding nucleotides of the target). Optionally, the oligomeric compounds, such as effector siRNA molecules of the multi-targeted molecules of the instant disclosure, contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

Complementary sequences within an effector molecule or a multi-targeted molecule, e.g., within a multi-targeted molecule as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsNA effector molecule of the instant disclosure comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

As used herein, the term “non-complementary nucleobase” refers to a pair of nucleobases that do not form hydrogen bonds with one another or otherwise support hybridization.

The terms “complementary,” “fully complementary” and “substantially complementary” herein can be used with respect to the base matching between the sense strand and the antisense strand of an effector molecule dsNA of the instant disclosure, or between the antisense strand of an effector molecule of the instant disclosure and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a target protein). For example, a polynucleotide is complementary to at least a part of a target mRNA if the sequence is substantially complementary to a non-interrupted portion of an mRNA encoding the target protein.

Accordingly, in some embodiments, the antisense strand polynucleotides disclosed herein are fully complementary to the target mRNA sequence.

In certain embodiments, the antisense strand polynucleotides disclosed herein are substantially complementary to the target RNA sequence and comprise a contiguous nucleotide sequence which is at least about 80% complementary over its entire length to the equivalent region of the nucleotide sequence of the target RNA, or a fragment of the target RNA, such as about 85%, about 86%, about 87%, about 88%, about 89%, about 90%, about 91%, about 92%, about 93%, about 94%, about 95%, about 96%, about 97%, about 98%, or about 99% complementary.

In one embodiment, an effector molecule that is a RNAi agent of a multi-targeted molecule of the disclosure includes a sense strand that is substantially complementary to an antisense polynucleotide which, in turn, is the same as a target RNA sequence, and wherein the sense strand polynucleotide comprises a contiguous nucleotide sequence which is at least about 80% complementary over its entire length to the equivalent region of the nucleotide sequence of the target RNA sequence, or a fragment of the target RNA sequence, such as about 85%, about 86%, about 87%, about 88%, about 89%, about 90%, about 91%, about 92%, about 93%, about 94%, about 95%, about 96%, about 97%, about 98%, or about 99% complementary.

In some embodiments, the double-stranded region of an effector molecule or sciRNA (or bis-sciRNA) agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of an effector molecule or sciRNA is equal to or at least 14, 15, 16, 17, 18, 19, 20, 21, 22, 23, 23, 24, 25, 26, 27, 28, 29, or 30 nucleotides in length.

In some embodiments, the sense strand of an effector molecule or sciRNA is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the effector molecule or sciRNA are each 15 to 30 nucleotides in length.

In one embodiment, the sense and antisense strands of the effector molecule or sciRNA are each 19 to 25 nucleotides in length.

In one embodiment, the sense and antisense strands of the effector molecule or sciRNA are each 21 to 23 nucleotides in length.

In some embodiments, one strand has at least one stretch of 1-5 single-stranded nucleotides in the double-stranded region. By “stretch of single-stranded nucleotides in the double-stranded region” is meant that there is present at least one nucleotide base pair at both ends of the single-stranded stretch. In some embodiments, both strands have at least one stretch of 1-5 (e.g., 1, 2, 3, 4, or 5) single-stranded nucleotides in the double stranded region. When both strands have a stretch of 1-5 (e.g., 1, 2, 3, 4, or 5) single-stranded nucleotides in the double stranded region, such single-stranded nucleotides can be opposite to each other (e.g., a stretch of mismatches) or they can be located such that the second strand has no single-stranded nucleotides opposite to the single-stranded iRNAs of the first strand and vice versa (e.g., a single-stranded loop). In some embodiments, the single-stranded nucleotides are present within 8 nucleotides from either end, for example, 8, 7, 6, 5, 4, 3, or 2 nucleotides from either the 5′ or 3′ end of the region of complementarity between the two strands.

In one embodiment, the effector molecule or the sciRNA comprises a single-stranded overhang on at least one of the termini. In one embodiment, the single-stranded overhang is 1, 2, or 3 nucleotides in length.

In one embodiment, the sense strand of the effector molecule or sciRNA is 21-nucleotides in length, and the antisense strand is 23-nucleotides in length, wherein the strands form a double-stranded region of 21 consecutive base pairs having a 2-nucleotide long single-stranded overhangs at the 3′-end.

In some embodiments, each strand of the effector molecule or sciRNA has a ZXY structure, such as is described in PCT Publication No. 2004080406, which is hereby incorporated by reference in its entirety.

In certain embodiment, the two nucleotide sequences can be linked together to form a long strand. The two nucleotide sequences can be linked together by an oligonucleotide linker including, but not limited to, (N)ₙ; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)₄, (U)₄, and (dT)₄, wherein N is a modified or unmodified nucleotide and R is a modified or unmodified purine nucleotide. Some of the nucleotides in the linker can be involved in base-pair interactions with other nucleotides in the linker. The two nucleotide sequences can also be linked together by a non-nucleotide based linker, e.g. a linker described herein. It will be appreciated by one of skill in the art that any oligonucleotide chemical modifications or variations describe herein can be used in the oligonucleotide linker.

In certain embodiments, two strands specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “hybridization” means the pairing of complementary oligomeric compounds (e.g., an antisense strand of an siRNA and its target nucleic acid or an antisense strand and sense strand of an siRNA). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In certain embodiments, the antisense strand of an siRNA specifically hybridizes to more than one target site.

As used herein, “designing” or “designed to” refer to the process of designing an oligomeric compound that specifically hybridizes with a selected nucleic acid molecule.

As used herein, the term “modulation” refers to a perturbation of function or activity when compared to the level of the function or activity prior to modulation. For example, modulation includes the change, either an increase (stimulation or induction) or a decrease (inhibition or reduction) in gene expression. As further example, modulation of expression can include perturbing splice site selection of pre-mRNA processing.

As used herein, the term “expression” refers to all the functions and steps by which a gene's coded information is converted into structures present and operating in a cell. Such structures include, but are not limited to the products of transcription and translation.

As used herein, “variant” refers to an alternative RNA transcript that can be produced from the same genomic region of DNA. Variants include, but are not limited to “pre-mRNA variants” which are transcripts produced from the same genomic DNA that differ from other transcripts produced from the same genomic DNA in either their start or stop position and contain both intronic and exonic sequence. Variants also include, but are not limited to, those with alternate splice junctions, or alternate initiation and termination codons.

As used herein, the term “2′-modified” or “2′-substituted” means a sugar comprising substituent at the 2′ position other than H or OH. 2′-modified monomers, include, but are not limited to, BNA's and monomers (e.g., nucleosides and nucleotides) with 2′-substituents, such as allyl, amino, azido, thio, O-allyl, O—C₁-C₁₀ alkyl, —OCF₃, O—(CH₂)₂—O-CH₃, 2′-O(CH₂)₂SCH₃, O—(CH₂)₂—O—N(R^{m})(Rⁿ), or O—CH₂—C(═O)—N(R^{m})(Rⁿ), where each R^{m} and Rⁿ is, independently, H or substituted or unsubstituted C₁-C₁₀ alkyl. In certain embodiments, oligomeric compounds comprise a 2′ modified monomer that does not have the formula 2′-O(CH₂)ₙH, wherein n is one to six. In certain embodiments, oligomeric compounds comprise a 2′ modified monomer that does not have the formula 2′-OCH₃. In certain embodiments, oligomeric compounds comprise a 2′ modified monomer that does not have the formula or, in the alternative, 2′-O(CH₂)₂OCH₃.

As used herein, the term “locked nucleic acid” or “LNA” or “locked nucleoside” or “locked nucleotide” refers to a nucleoside or nucleotide wherein the furanose portion of the nucleoside includes a bridge connecting two carbon atoms on the furanose ring, thereby forming a bicyclic ring system. Locked nucleic acids are also referred to as bicyclic nucleic acids (BNA).

As used herein, unless otherwise indicated, the term “methyleneoxy LNA” alone refers to β-D-methyleneoxy LNA.

As used herein, the term “MOE” refers to a 2′-O-methoxyethyl substituent.

As used herein, the term “gapmer” refers to a chimeric oligomeric compound comprising a central region (a “gap”) and a region on either side of the central region (the “wings”), wherein the gap comprises at least one modification that is different from that of each wing. Such modifications include nucleobase, monomeric linkage, and sugar modifications as well as the absence of modification (unmodified). Thus, in certain embodiments, the nucleotide linkages in each of the wings are different than the nucleotide linkages in the gap. In certain embodiments, each wing comprises nucleotides with high affinity modifications and the gap comprises nucleotides that do not comprise that modification. In certain embodiments the nucleotides in the gap and the nucleotides in the wings all comprise high affinity modifications, but the high affinity modifications in the gap are different than the high affinity modifications in the wings. In certain embodiments, the modifications in the wings are the same as one another. In certain embodiments, the modifications in the wings are different from each other. In certain embodiments, nucleotides in the gap are unmodified and nucleotides in the wings are modified. In certain embodiments, the modification(s) in each wing are the same. In certain embodiments, the modification(s) in one wing are different from the modification(s) in the other wing. In certain embodiments, oligomeric compounds are gapmers having 2′-deoxynucleotides in the gap and nucleotides with high-affinity modifications in the wing.

As used herein, the term “prodrug” refers to a therapeutic agent that is prepared in an inactive form that is converted to an active form (i.e., drug) within the body or cells thereof by the action of endogenous enzymes or other chemicals and/or conditions.

As used herein, the term “pharmaceutically acceptable salts” refers to salts of active compounds that retain the desired biological activity of the active compound and do not impart undesired toxicological effects thereto.

As used herein, the term “cap structure” or “terminal cap moiety” refers to chemical modifications, which have been incorporated at either terminus of an antisense compound.

The term “substituted” refers to the replacement of one or more hydrogen radicals in a given structure with the radical of a specified substituent including, but not limited to: alkyl, alkenyl, alkynyl, aryl, heterocyclyl, halo, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent can be further substituted.

The term “alkyl” refers to saturated and unsaturated non-aromatic hydrocarbon chains that may be a straight chain or branched chain, containing the indicated number of carbon atoms (these include without limitation propyl, allyl, or propargyl), which may be optionally inserted with N, O, or S. For example, C1-C10 indicates that the group may have from 1 to 10 (inclusive) carbon atoms in it. In certain embodiments, a lipophilic moiety of the instant disclosure can include a C₆-C₁₈ alkyl hydrocarbon chain. The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene. The term “aminoalkyl” refers to an alkyl group as defined above, substituted at any position with one or more amino groups as permitted by normal valency. The amino groups may be unsubstituted, monosubstituted, or di-substituted.

The term “mercapto” refers to an —SH radical. The term “thioalkoxy” refers to an —S— alkyl radical.

The term “halo” refers to any radical of fluorine, chlorine, bromine or iodine.

As used herein, the term “cycloalkyl” means a saturated or unsaturated nonaromatic hydrocarbon ring group having from 3 to 14 carbon atoms, unless otherwise specified. Examples of cycloalkyl groups include, but are not limited to, cyclopropyl, methyl-cyclopropyl, 2,2-dimethyl-cyclobutyl, 2-ethyl-cyclopentyl, cyclohexyl, etc. Cycloalkyls may include multiple spiro- or fused rings. Cycloalkyl groups are optionally mono-, di-, tri-, tetra-, or penta-substituted on any position as permitted by normal valency.

As used herein, the term “alkenyl” refers to a non-aromatic hydrocarbon radical, straight or branched, containing at least one carbon-carbon double bond, and having from 2 to 10 carbon atoms unless otherwise specified. Up to five carbon-carbon double bonds may be present in such groups. For example, “C₂-C₆” alkenyl is defined as an alkenyl radical having from 2 to 6 carbon atoms. Examples of alkenyl groups include, but are not limited to, ethenyl, propenyl, butenyl, and cyclohexenyl. The straight, branched, or cyclic portion of the alkenyl group may contain double bonds and is optionally mono-, di-, tri-, tetra-, or penta-substituted on any position as permitted by normal valency. The term “cycloalkenyl” means a monocyclic hydrocarbon group having the specified number of carbon atoms and at least one carbon-carbon double bond.

As used herein, the term “alkynyl” refers to a hydrocarbon radical, straight or branched, containing from 2 to 10 carbon atoms, unless otherwise specified, and containing at least one carbon-carbon triple bond. Up to 5 carbon-carbon triple bonds may be present. Thus, “C2-C6 alkynyl” means an alkynyl radical having from 2 to 6 carbon atoms. Examples of alkynyl groups include, but are not limited to, ethynyl, 2-propynyl, and 2-butynyl. The straight or branched portion of the alkynyl group may contain triple bonds as permitted by normal valency, and may be optionally mono-, di-, tri-, tetra-, or penta-substituted on any position as permitted by normal valency.

As used herein, “alkoxyl” or “alkoxy” refers to an alkyl group as defined above with the indicated number of carbon atoms attached through an oxygen bridge. C₁₋₆ alkoxy, is intended to include C1, C2, C3, C4, C5, and C6 alkoxy groups. C1-8 alkoxy, is intended to include C1, C2, C3, C4, C5, C6, C7, and C8 alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” or “aromatic” means any stable monocyclic or polycyclic carbon ring of up to 7 atoms in each ring, wherein at least one ring is aromatic. Examples of aryl groups include, but are not limited to, phenyl, naphthyl, anthracenyl, tetrahydronaphthyl, indanyl, and biphenyl. In cases where the aryl substituent is bicyclic and one ring is non-aromatic, it is understood that attachment is via the aromatic ring. Aryl groups are optionally mono-, di-, tri-, tetra-, or penta-substituted on any position as permitted by normal valency. The term “arylalkyl” or the term “aralkyl” refers to alkyl substituted with an aryl. The term “arylalkoxy” refers to an alkoxy substituted with aryl.

As used herein, the term “heteroaryl” represents a stable monocyclic or polycyclic ring of up to 7 atoms in each ring, wherein at least one ring is aromatic and contains from 1 to 4 heteroatoms selected from the group consisting of O, N and S. Examples of heteroaryl groups include, but are not limited to, acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, tetrahydroquinoline. “Heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring. Heteroaryl groups are optionally mono-, di-, tri-, tetra-, or penta-substituted on any position as permitted by normal valency.

As used herein, the term “heterocycle,” “heterocyclic,” or “heterocyclyl” means a 3- to 14-membered aromatic or nonaromatic heterocycle containing from 1 to 4 heteroatoms selected from the group consisting of O, N and S, including polycyclic groups. As used herein, the term “heterocyclic” is also considered to be synonymous with the terms “heterocycle” and “heterocyclyl” and is understood as also having the same definitions set forth herein. “Heterocyclyl” includes the above mentioned heteroaryls, as well as dihydro and tetrahydro analogs thereof. Examples of heterocyclyl groups include, but are not limited to, azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyridinonyl, pyrimidyl, pyrimidinonyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom. Heterocyclyl groups are optionally mono-, di-, tri-, tetra-, or penta-substituted on any position as permitted by normal valency.

“Heterocycloalkyl” refers to a cycloalkyl residue in which one to four of the carbons is replaced by a heteroatom such as oxygen, nitrogen or sulfur. Examples of heterocycles whose radicals are heterocyclyl groups include tetrahydropyran, morpholine, pyrrolidine, piperidine, thiazolidine, oxazole, oxazoline, isoxazole, dioxane, tetrahydrofuran and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “cycloalkyl” as employed herein includes saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Cycloalkyl groups include, without limitation, cyclopropyl, cyclobutyl, cyclopentyl, cyclopentenyl, cyclohexyl, cyclohexenyl, cycloheptyl, and cyclooctyl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include trizolyl, tetrazolyl, piperazinyl, pyrrolidinyl, dioxanyl, morpholinyl, tetrahydrofuranyl, and the like.

The term “acyl” refers to an alkylcarbonyl, cycloalkylcarbonyl, arylcarbonyl, heterocyclylcarbonyl, or heteroarylcarbonyl substituent, any of which may be further substituted by substituents.

As used herein, “keto” refers to any alkyl, alkenyl, alkynyl, cycloalkyl, cycloalkenyl, heterocyclyl, heteroaryl, or aryl group as defined herein attached through a carbonyl bridge.

Examples of keto groups include, but are not limited to, alkanoyl (e.g., acetyl, propionyl, butanoyl, pentanoyl, hexanoyl), alkenoyl (e.g., acryloyl) alkynoyl (e.g., ethynoyl, propynoyl, butynoyl, pentynoyl, hexynoyl), aryloyl (e.g., benzoyl), heteroaryloyl (e.g., pyrroloyl, imidazoloyl, quinolinoyl, pyridinoyl).

As used herein, “alkoxycarbonyl” refers to any alkoxy group as defined above attached through a carbonyl bridge (i.e., —C(O)O-alkyl). Examples of alkoxycarbonyl groups include, but are not limited to, methoxycarbonyl, ethoxycarbonyl, iso-propoxycarbonyl, n-propoxycarbonyl, t-butoxycarbonyl, benzyloxycarbonyl or n-pentoxycarbonyl.

As used herein, “aryloxycarbonyl” refers to any aryl group as defined herein attached through an oxycarbonyl bridge (i.e., —C(O)O-aryl). Examples of aryloxycarbonyl groups include, but are not limited to, phenoxycarbonyl and naphthyloxycarbonyl.

As used herein, “heteroaryloxycarbonyl” refers to any heteroaryl group as defined herein attached through an oxycarbonyl bridge (i.e., —C(O)O-heteroaryl). Examples of heteroaryloxycarbonyl groups include, but are not limited to, 2-pyridyloxycarbonyl, 2-oxazolyloxycarbonyl, 4-thiazolyloxycarbonyl, or pyrimidinyloxycarbonyl.

The term “oxo” refers to an oxygen atom, which forms a carbonyl when attached to carbon, an N-oxide when attached to nitrogen, and a sulfoxide or sulfone when attached to sulfur.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the pH of the environment, as would be readily understood by the person of ordinary skill in the art.

As used herein, the term “prevention” refers to delaying or forestalling the onset or development of a condition or disease for a period of time from hours to days, preferably weeks to months. As such, “prevention” or “preventing” can herein be used in reference to a disease or disorder in a subject that would benefit from a reduction in expression of a target gene(s) or production of target gene protein(s), e.g., in a subject susceptible to a target gene-associated disorder due to, e.g., genetic factors or age, wherein the subject does not yet meet the diagnostic criteria for the target gene-associated disorder. As used herein, prevention can be understood as administration of an agent to a subject who does not yet meet the diagnostic criteria for the target gene-associated disorder, to delay or reduce the likelihood that the subject will develop the target gene-associated disorder. As the agent is a pharmaceutical agent, it is understood that administration typically would be under the direction of a health care professional capable of identifying a subject who does not yet meet the diagnostic criteria for a target gene-associated disorder as being susceptible to developing a target gene-associated disorder.

As used herein, the term “amelioration” refers to a lessening of at least one activity or one indicator of the severity of a condition or disease. The severity of indicators may be determined by subjective or objective measures which are known to those skilled in the art.

As used herein, the term “treatment” refers to administering a composition of the present disclosure to affect an alteration or improvement of the disease or condition. Prevention, amelioration, and/or treatment may require administration of multiple doses at regular intervals, or prior to onset of the disease or condition to alter the course of the disease or condition. Moreover, a single agent may be used in a single individual for each prevention, amelioration, and treatment of a condition or disease sequentially, or concurrently. The terms “treating” or “treatment” accordingly refer to a beneficial or desired result including, but not limited to, alleviation or amelioration of one or more signs or symptoms associated with target gene expression or target gene protein production, e.g., a target gene-associated CNS disease or disorder as described elsewhere herein, by achieving decreased expression or activity of target gene(s) in regions of increased neuronal dysfunction or death, in subjects having such CNS diseases or disorders. “Treatment” can also mean prolonging survival as compared to expected survival in the absence of treatment.

As used herein, the term “pharmaceutical agent” refers to a substance that provides a therapeutic benefit when administered to a subject. In certain embodiments, a pharmaceutical agent is an active pharmaceutical agent. In certain embodiments, a pharmaceutical agent is a prodrug.

As used herein, the term “therapeutically effective amount” refers to an amount of a pharmaceutical agent that provides a therapeutic benefit to an animal.

As used herein, “administering” means providing a pharmaceutical agent to an animal, and includes, but is not limited to administering by a medical professional and self-administering.

As used herein, the term “co-administering” means providing more than one pharmaceutical agent to an animal. In certain embodiments, such more than one pharmaceutical agents are administered together. In certain embodiments, such more than one pharmaceutical agents are administered separately. In certain embodiments, such more than one pharmaceutical agents are administered at the same time. In certain embodiments, such more than one pharmaceutical agents are administered at different times. In certain embodiments, such more than one pharmaceutical agents are administered through the same route of administration. In certain embodiments, such more than one pharmaceutical agents are administered through different routes of administration. In certain embodiments, such more than one pharmaceutical agents are contained in the same pharmaceutical formulation. In certain embodiments, such more than one pharmaceutical agents are in separate formulations.

As used herein, the term “pharmaceutical composition” refers to a mixture of substances suitable for administering to an individual. For example, a pharmaceutical composition may comprise a multi-targeted molecule as described herein and a sterile aqueous solution. In certain embodiments, a pharmaceutical composition includes a pharmaceutical agent and a diluent and/or carrier.

As used herein, the term “in vitro” refers to events that occur in an artificial environment, e.g., in a test tube or reaction vessel, in cell culture, etc., rather than within an organism (e.g. animal or a plant). As used herein, the term “ex vivo” refers to cells which are removed from a living organism and cultured outside the organism (e.g., in a test tube). As used herein, the term “in vivo” refers to events that occur within an organism (e.g. animal, plant, and/or microbe).

As used herein, the term “subject” or “patient” refers to any organism to which a composition disclosed herein can be administered, e.g., for experimental, diagnostic, and/or therapeutic purposes. Typical subjects include animals (e.g., mammals such as mice, rats, rabbits, non-human primates, and humans) and/or plants. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “parenteral administration,” refers to administration through injection or infusion. Parenteral administration includes, but is not limited to, subcutaneous administration, intravenous administration, or intramuscular administration.

As used herein, the term “subcutaneous administration” refers to administration just below the skin. “Intravenous administration” means administration into a vein.

As used herein, the term “intracerebroventricular injection” or “ICV injection” or “ICV administration” refers to an injection technique of substances directly into the cerebrospinal fluid in cerebral ventricles, thereby bypassing the blood brain barrier (BBB).

As used herein, the term “intrathecal administration” or “intrathecal injection” refers to a route of administration for drugs via an injection into the spinal canal, or into the subarachnoid space so that it reaches the cerebrospinal fluid (CSF), which is commonly useful in spinal anesthesia, chemotherapy, pain management applications, as well as for treating certain infections, particularly post-neurosurgical.

As used herein, the term “dose” refers to a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

As used herein, the term “dosage unit” refers to a form in which a pharmaceutical agent is provided. In certain embodiments, a dosage unit is a vial comprising lyophilized multi-targeted molecule as described herein. In certain embodiments, a dosage unit is a vial comprising reconstituted multi-targeted molecule as described herein.

As used herein, the term “active pharmaceutical ingredient” refers to the substance in a pharmaceutical composition that provides a desired effect.

As used herein, the term “side effects” refers to physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

The terms “bis(siRNA)” and “bis-siRNA” are used interchangeably herein and refer to two siRNAs covalently or non-covalently linked to form a single chemical entity that can elicit RNAi activity in vitro or in vivo. The two linked siRNAs can target the same target gene at different sites or the two linked siRNAs can target can respectively target sites in two different genes (mRNAs) to elicit RNAi-mediated gene silencing.

For clarification, one of the siRNAs in the bis(siRNA) can modulate gene expression of a first target nucleic acid and the other siRNA in the bis(siRNA) can modulate gene expression of a second target nucleic acid. In some embodiments, the first and second target nucleic acids are the same.

In some other embodiments, one of the siRNAs in the bis(siRNA) can modulate gene expression of a first target nucleic acid and the other siRNA in the bis(siRNA) can modulate gene expression of a second target nucleic acid, where the first and the second target nucleic acids are different genes.

As used herein, “linker” or “linkers” includes nucleotide and non-nucleotide linkers or combinations thereof that connects two parts of a molecule, for example, one or both strands of two individual siRNA molecule to generate a bis(siRNA). In some embodiments, mere electrostatic or stacking interaction between two individual siRNAs can represent a linker. The non-nucleotide linkers include tether or linker derived from monosaccharide, disaccharides, oligosaccharides and derivatives thereof, aliphatic, alicyclic, heterocyclic and combinations thereof. In certain embodiments, the term “linker” means an organic moiety that connects two parts of a compound, such an organic moiety that connects two oligonucleotide molecules. Linkers typically comprise an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl,

alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases. For example, the linker can be cleaved by a metabolic enzyme in vitro and/or in vivo. Exemplary metabolic enzymes include, but are not limited to classes of nucleases, proteases, peptidases, glycosylases, glycosydases, hydrolyses, oxidases, etc.

In general, a multi-targeted molecule as described herein can include ribonucleotides, but as described in detail herein, each or both strands can also include one or more non-ribonucleotides, e.g., a deoxyribonucleotide, a modified nucleotide. In addition, as used in this specification, a multi-targeted molecule of the instant disclosure may include ribonucleotides with chemical modifications; a multi-targeted molecule of the instant disclosure may include substantial modifications at multiple nucleotides, as described in additional detail herein.

As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA-type molecule (e.g., in effector molecules of the multi-targeted molecules of the instant disclosure), or in oligonucleotide linker compositions, are encompassed by “multi-targeted molecule” for the purposes of this specification and claims.

In certain embodiments of the instant disclosure, inclusion of a deoxy-nucleotide—which is acknowledged as a naturally occurring form of nucleotide—if present within a multi-targeted molecule of the instant disclosure (e.g., in RNAi effector molecules or oligonucleotide linkers of multi-targeted molecules as disclosed herein) can be considered to constitute a modified nucleotide.

The duplex region of an effector molecule of a multi-targeted molecule of the instant disclosure may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the present disclosure.

Within an individual effector molecule that is double-stranded (noting that in certain embodiments, it is also contemplated that an effector molecule that is not double-stranded may be included in a multi-targeted molecule of the instant disclosure), the two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiments, within an individual effector molecule of a multi-targeted molecule of the instant disclosure, the two strands of a double-stranded oligomeric compound of an effector molecule can be linked together (in such embodiments, such linkers are separate and apart from the linker(s) contemplated herein for joining of individual effector molecules to one another). The two strands can be linked to each other at both ends, or at one end only. By linking at one end is meant that 5′-end of first strand is linked to the 3′-end of the second strand or 3′-end of first strand is linked to 5′-end of the second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleosidic linker, e.g. a linker described herein. It will be appreciated by one of skill in the art that any oligonucleotide chemical modifications or variations describe herein can be used in the oligonucleotide linker.

In embodiments where individual effector molecules are hairpin- or dumbbell-type oligomeric compounds, such molecules will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

In such embodiments, hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an effector molecule may comprise one or more nucleotide overhangs.

In one embodiment, an effector molecule of a multi-targeted molecule of the instant disclosure is a dsRNA, each strand of which is 24-30 nucleotides in length, that interacts with a target RNA sequence to direct the cleavage of the target RNA.

In another embodiment, an effector molecule of a multi-targeted molecule of the instant disclosure is a dsRNA agent, each strand of which comprises 19-23 nucleotides that interacts with a target RNA sequence to direct the cleavage of the target RNA.

As used herein, the term “nucleotide overhang” refers to at least one unpaired nucleotide that protrudes from the duplex structure of a double-stranded nucleic acid (dsNA). For example, when a 3′-end of one strand of a dsNA extends beyond the 5′-end of the other strand, or vice versa, there is a nucleotide overhang. A dsNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsNA. In certain embodiments, it is specifically contemplated that a nucleotide overhang can exist where one or more terminal nucleotides of one strand of an effector molecule do not base-pair with a corresponding one or more nucleotides of a linker molecule (e.g., where the linker molecule(s) of the multi-targeted molecule that joins effector molecules is not considered as a structural part of the individual effector molecule).

In one embodiment, at least one strand of an effector molecule of a multi-targeted molecule of the instant disclosure comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the effector molecule of the multi-targeted molecule comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the effector molecule of the multi-targeted molecule comprise an overhang of at least 1 nucleotide.

In one embodiment, the antisense strand of an effector molecule of the instant disclosure has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end, the 5′-end, at both ends, or at neither end. In one embodiment, the sense strand of an effector molecule of the instant disclosure has a 1-10 nucleotide, e.g., a 1, 2, 3, 4, 5, 6, 7, 8, 9, or 10 nucleotide, overhang at the 3′-end, the 5′-end, at both ends, or at neither end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended” as used herein in reference to an effector molecule or a multi-targeted molecule of the instant disclosure refer to structures having no unpaired nucleotides or nucleotide analogs at a given terminal end of a dsNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. In embodiments, such a molecule will be double stranded over its entire length.

The term “antisense strand” or “guide strand” refers to the strand of an effector molecule that is an RNAi agent, e.g., a dsRNA, which includes a region that is substantially complementary to a target sequence, e.g., a target mRNA.

As used herein, the term “region of complementarity” refers to the region on the antisense strand that is substantially complementary to a sequence, for example, a target sequence, as defined herein. Where the region of complementarity is not fully complementary to the target sequence, the mismatches can be in the internal (non-terminal) or terminal regions of the molecule. Generally, the most tolerated mismatches within an effector molecule of the multi-targeted molecules of the instant disclosure are in the terminal regions, e.g., within 5, 4, 3, or 2 nucleotides of the 5′- or 3′-terminus of the individual effector molecule.

In some embodiments, an effector molecule of a multi-targeted molecule of the instant disclosure includes a nucleotide mismatch in the antisense strand.

In some embodiments, the antisense strand of an effector molecule of a multi-targeted molecule of the instant disclosure includes no more than 4 mismatches with the target mRNA, e.g., the antisense strand includes 4, 3, 2, 1, or 0 mismatches with the target mRNA. In some embodiments, the antisense strand of an effector molecule of a multi-targeted molecule of the instant disclosure includes no more than 4 mismatches with the sense strand, e.g., the antisense strand includes 4, 3, 2, 1, or 0 mismatches with the sense strand. In some embodiments, an effector molecule of a multi-targeted molecule of the instant disclosure includes a nucleotide mismatch in the sense strand. In some embodiments, the sense strand of the effector molecule of a multi-targeted molecule of the instant disclosure includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of an individual effector molecule of a multi-targeted molecule of the instant disclosure. In another embodiment, the nucleotide mismatch is, for example, in the 3′-terminal nucleotide of the effector molecule that is an iRNA agent. In some embodiments, the mismatch(s) is not in the seed region.

Thus, an effector molecule of a multi-targeted molecule as described herein can contain one or more mismatches to the target sequence(s) of the effector molecule(s). In one embodiment, an effector molecule of a multi-targeted molecule as described herein contains no more than 3 mismatches (i.e., 3, 2, 1, or 0 mismatches). In one embodiment, an effector molecule of a multi-targeted molecule as described herein contains no more than 2 mismatches. In one embodiment, an effector molecule of a multi-targeted molecule as described herein contains no more than 1 mismatch. In one embodiment, an effector molecule of a multi-targeted molecule as described herein contains 0 mismatches. In certain embodiments, if the antisense strand of the effector molecule of a multi-targeted molecule as described herein contains mismatches to the target sequence, the mismatch can optionally be restricted to be within the last 5 nucleotides from either the 5′- or 3′-end of the region of complementarity. For example, in such embodiments, for a 23 nucleotide effector molecule strand of a multi-targeted molecule as described herein, the strand which is complementary to a region of a target gene generally does not contain any mismatch within the central 13 nucleotides. The methods described herein or methods known in the art can be used to determine whether an effector molecule containing a mismatch to a target sequence is effective in inhibiting the expression of a target gene. Consideration of the efficacy of an effector molecule with mismatches in inhibiting expression of a target gene is important, especially if the particular region of complementarity in a target gene is known to have polymorphic sequence variation within the population.

The term “sense strand” or “passenger strand” as used herein, refers to the strand of an effector molecule, where the effector molecule is a RNAi agent, that includes a region that is substantially complementary to a region of the antisense strand as that term is defined herein.

As used herein, “substantially all of the nucleotides are modified” refers to effector molecules or multi-targeted molecules that are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

In some embodiments, the double-stranded region of an effector molecule that is a double-stranded nucleic acid (e.g., a double-stranded iRNA agent) is equal to or at least 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of an effector molecule that is a double-stranded nucleic acid (e.g., a double-stranded iRNA agent) is equal to or at least 14, 15, 16, 17, 18, 19, 20, 21, 22, 23, 23, 24, 25, 26, 27, 28, 29, or 30 nucleotides in length.

In some embodiments, the sense strand of an effector molecule that is a double-stranded nucleic acid (e.g., a double-stranded iRNA agent) is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the effector molecule that is a double-stranded nucleic acid (e.g., a double-stranded iRNA agent) are each 15 to 30 nucleotides in length.

In one embodiment, the sense and antisense strands of the effector molecule that is a double-stranded nucleic acid (e.g., a double-stranded iRNA agent) are each 19 to 25 nucleotides in length.

In one embodiment, the sense and antisense strands of the effector molecule that is a double-stranded nucleic acid (e.g., a double-stranded iRNA agent) are each 21 to 23 nucleotides in length.

In one embodiment, the sense strand of the effector molecule that is a double-stranded nucleic acid (e.g., a double-stranded iRNA agent) is 21-nucleotides in length, and the antisense strand is 23-nucleotides in length, wherein the strands form a double-stranded region of 21 consecutive base pairs having a 2-nucleotide long single stranded overhangs at the 3-end.

In some embodiments, the majority of nucleotides of each strand are ribonucleotides, but as described in detail herein, each or both strands can also include one or more non-ribonucleotides, e.g., a deoxyribonucleotide or a modified nucleotide. In addition, the effector molecule that is a double-stranded nucleic acid (e.g., a double-stranded iRNA agent) may include ribonucleotides with chemical modifications. Such modifications may include all types of modifications disclosed herein or known in the art. Any such modifications, as used in an effector molecule that is a double-stranded nucleic acid (e.g., a double-stranded iRNA agent), are encompassed by an “effector molecule” of a multi-targeted molecule as disclosed herein, for the purposes of this specification and claims.

The phrase “contacting a cell with a multi-targeted molecule,” as used herein, includes contacting a cell by any possible means. Contacting a cell with a multi-targeted molecule includes contacting a cell in vitro with the multi-targeted molecule or contacting a cell in vivo with the multi-targeted molecule. The contacting may be done directly or indirectly. Thus, for example, the multi-targeted molecule may be put into physical contact with the cell by the individual performing the method, or alternatively, the multi-targeted molecule may be put into a situation that will permit or cause it to subsequently come into contact with the cell.

Contacting a cell in vitro may be done, for example, by incubating the cell with the multi-targeted molecule. Contacting a cell in vivo may be done, for example, by injecting the multi-targeted molecule into or near the tissue where the cell is located, or by injecting the multi-targeted molecule into another area, e.g., the central nervous system (CNS), optionally via intrathecal, intravitreal or other injection, or to the bloodstream or the subcutaneous space, such that the agent will subsequently reach the tissue where the cell to be contacted is located. For example, the multi-targeted molecule may contain or be coupled to a ligand, e.g., a lipophilic moiety or moieties as described below and further detailed, e.g., in PCT/US2019/031170, which is incorporated herein by reference, that directs or otherwise stabilizes the multi-targeted molecule at a site of interest, e.g., the CNS. Combinations of in vitro and in vivo methods of contacting are also possible. For example, a cell may also be contacted in vitro with a multi-targeted molecule and subsequently transplanted into a subject.

In one embodiment, contacting a cell with a multi-targeted molecule includes “introducing” or “delivering the multi-targeted molecule into the cell” by facilitating or effecting uptake or absorption into the cell. Absorption or uptake of a multi-targeted molecule can occur through unaided diffusive or active cellular processes, or by auxiliary agents or devices. Introducing a multi-targeted molecule into a cell may be in vitro or in vivo. For example, for in vivo introduction, a multi-targeted molecule can be injected into a tissue site or administered systemically. In vitro introduction into a cell includes methods known in the art such as electroporation and lipofection. Further approaches are described herein below or are known in the art.

The term “lipophile” or “lipophilic moiety” broadly refers to any compound or chemical moiety having an affinity for lipids. One way to characterize the lipophilicity of the lipophilic moiety is by the octanol-water partition coefficient, log Kow, where Kow is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41: 1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log Kow exceeds 0. Typically, the lipophilic moiety possesses a log Kow exceeding 1, exceeding 1.5, exceeding 2, exceeding 3, exceeding 4, exceeding 5, or exceeding 10. For instance, the log Kow of 6-amino hexanol, for instance, is predicted to be approximately 0.7. Using the same method, the log Kow of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., log Kow) value of the lipophilic moiety.

Alternatively, the hydrophobicity of the multi-targeted molecule of the instant disclosure, possessing/conjugated to multiple lipophilic moieties, can be measured by its protein binding characteristics. For instance, in certain embodiments, the unbound fraction in the plasma protein binding assay of the multi-targeted molecule could be determined to positively correlate to the relative hydrophobicity of the multi-targeted molecule, which could then positively correlate to the silencing activity of the multi-targeted molecule.

In one embodiment, the plasma protein binding assay for determining hydrophobicity is an electrophoretic mobility shift assay (EMSA) using human serum albumin protein. An exemplary protocol of this binding assay is illustrated in detail in, e.g., PCT/US2019/031170. The hydrophobicity of the multi-targeted molecule, measured by fraction of unbound multi-targeted molecule in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of a multi-targeted molecule.

Accordingly, conjugating the lipophilic moieties to the internal (non-terminal) position(s) of the effector molecule(s) of a multi-targeted molecule of the instant disclosure provides optimal hydrophobicity for the enhanced in vivo delivery of a multi-targeted molecule.

The term “lipid nanoparticle” or “LNP” refers to a vesicle comprising a lipid layer encapsulating a pharmaceutically active molecule, such as a nucleic acid molecule, e.g., a multi-targeted molecule or a plasmid from which a multi-targeted molecule is transcribed. LNPs are described in, for example, U.S. Pat. Nos. 6,858,225, 6,815,432, 8,158,601, and 8,058,069, the entire contents of which are hereby incorporated herein by reference.

The term “lower” in the context of the level of target gene in a subject or a disease marker or symptom refers to a statistically significant decrease in such level. The decrease can be, for example, at least 10%, 15%, 20%, 25%, 30%, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of target gene in a subject is optionally down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in speed of movement (bradykinesia) and ability to regulate posture and balance in an individual having Parkinson's and an individual not having Parkinson's or having symptoms that are within the range of normal.

“Therapeutically effective amount,” as used herein, is intended to include the amount of a multi-targeted molecule that, when administered to a subject having a target gene-associated disease, is sufficient to effect treatment of the disease (e.g., by diminishing, ameliorating, or maintaining the existing disease or one or more symptoms of disease). The “therapeutically effective amount” may vary depending on the multi-targeted molecule, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of a multi-targeted molecule that, when administered to a subject having a target gene-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the multi-targeted molecule, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of a multi-targeted molecule that produces some desired local or systemic effect at a reasonable benefit/risk ratio applicable to any treatment. A multi-targeted molecule employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain, e.g., striatum, or certain types of cells in the brain, such as, e.g., neurons and glial cells (astrocytes, oligodendrocytes, microglial cells)). In other embodiments, a “sample derived from a subject” refers to liver tissue (or subcomponents thereof) derived from the subject. In some embodiments, a “sample derived from a subject” refers to blood drawn from the subject or plasma or serum derived therefrom. In further embodiments, a “sample derived from a subject” refers to brain tissue (or subcomponents thereof) or retinal tissue (or subcomponents thereof) derived from the subject.

### Multi-Targeted Molecule Sequences Design

In one embodiment, the effector molecule or sciRNA agent comprises a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the effector molecule or sciRNA agent comprises a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the effector molecule or sciRNA agent comprises a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the effector molecule or sciRNA agent comprises a 21 nucleotides (nt) sense strand and a 23 nucleotides (nt) antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the effector molecule or sciRNA is blunt, while the other end is comprises a 2 nt overhang. Preferably, the 2 nt overhang is at the 3′-end of the antisense strand. Optionally, the effector molecule or sciRNA agent further comprises a ligand (e.g., GalNAc₃).

In one embodiment, the effector molecule or sciRNA agent comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the effector molecule or the sciRNA agent comprises a sense and antisense strands, wherein said effector molecule or sciRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region region which is at least 25 nucleotides in length, and said second strand is sufficiently complemenatary to a target mRNA along at least 19 nt of said second strand length to reduce target gene expression when said effector molecule or sciRNA agent is introduced into a mammalian cell, and wherein dicer cleavage of said effector molecule or sciRNA preferentially results in an siRNA comprising said 3′ end of said second strand, thereby reducing expression of the target gene in the mammal. Optionally, the effector molecule or sciRNA agent further comprises a ligand (e.g., GalNAc₃).

In one embodiment, the sense strand of effector molecule or the sciRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand of the effector molecule or sciRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For effector molecule or sciRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the effector molecule or sciRNA from the 5′-end.

In some embodiments, the effector molecule or sciRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the effector molecule or sciRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the effector molecule or sciRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′end.

In one embodiment, the effector molecule or sciRNA agent comprises mismatch(es) with the target, within the duplex, or combinations thereof. The mistmatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the effector molecule or sciRNA agent comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In some embodiments, provided herein are an effector molecule or an sciRNA (or bis-sciRNA) agent. The effector molecule is a double-stranded RNA (dsRNA) agent, i.e., siRNA, for inhibiting the expression of a target gene. It is understood that dsRNA, siRNA, oligonucleotides can be used interchangeably unless otherwise stated.

The effector molecule (dsRNA agent) or the sciRNA agent comprises a sense strand and an antisense strand, each strand having 14 to 45 nucleotides. The effector molecule (dsRNA agent) or the sciRNA agent is represented by formula (I):

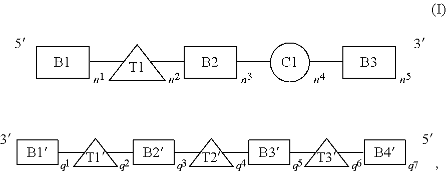

In formula (I), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

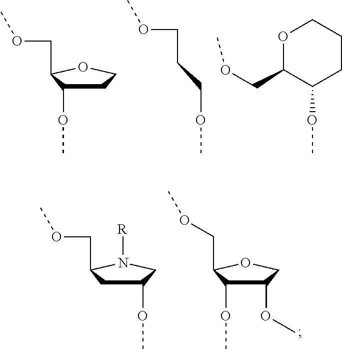

and iii) sugar modification selected from the group consisting of:

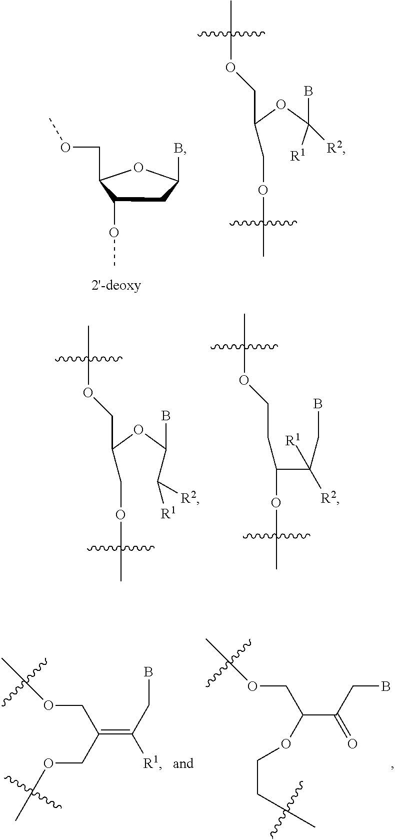

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

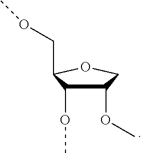

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1, T1′, T2′, and T3′ are each independently 2′-F. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0. q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In some embodiments, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In some embodiments, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, 100%, 95%, 90%, 85%, 80%, 75%, 70%, 65%, 60%, 55%, 50%, 45%, 40%, 35% or 30% of the effector molecule (dsRNA agent) or sciRNA agent is modified. For example, when 50% of the effector molecule (dsRNA agent) or the sciRNA agent is modified, 50% of all nucleotides present in effector molecule (dsRNA agent) or the sciRNA agent contain a modification as described herein.

In one embodiment, each of the sense and antisense strands of the effector molecule (dsRNA agent) or the sciRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In one embodiment, each of the sense and antisense strands of the dsRNA agent or the sciRNA (or bis-sciRNA) agent contains at least two different modifications.

In one embodiment, the effector molecule (dsRNA agent) or the sciRNA agent of Formula (I) further comprises 3′ and/or 5′ overhang(s) of 1-10 nucleotides in length. In one example, the effector molecule (dsRNA agent) or the sciRNA agent of formula (I) comprises a 3′ overhang at the 3′-end of the antisense strand and a blunt end at the 5′-end of the antisense strand. In another example, the effector molecule (dsRNA agent) or the sciRNA agent has a 5′ overhang at the 5′-end of the sense strand.

In one embodiment, the effector molecule (dsRNA agent) or the sciRNA agent does not contain any 2′-F modification.

In one embodiment, the sense strand and/or antisense strand of the effector molecule (dsRNA agent) or the sciRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the effector molecule (dsRNA agent) or the sciRNA agent is 100% complementary to a target RNA to hybridize thereto and inhibits its expression through RNA interference. In another embodiment, the antisense strand of the effector molecule (dsRNA agent) or the sciRNA agent is at least 95%, at least 90%, at least 85%, at least 80%, at least 75%, at least 70%, at least 65%, at least 60%, at least 55%, or at least 50% complementary to a target RNA.

Additional exemplary embodiments for the sequence designs of the effector molecule (dsRNA agent) or the sciRNA agent represented by formula (I) can be found in WO 2016/028649, which is incorporated herein by reference in its entirety.

The effector molecule (dsRNA agent) or the sciRNA agent can comprise a phosphorus-containing group at the 5′-end of a sense or antisense nucleotide sequence. The 5′-end phosphorus-containing group can be 5′-end phosphate (5′-P), 5′-end phosphorothioate (5′-PS), 5′-end phosphorodithioate (5′-PS2), 5′-end vinylphosphonate (5′-VP), 5′-end methylphosphonate (MePhos), or 5′-deoxy-5′-C-malonyl

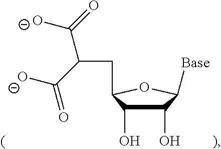

When the 5′-end phosphorus-containing group is 5′-end vinylphosphonate (5′-VP), the 5′-VP can be either 5′-E-VP isomer (i.e., trans-vinylphosphate,

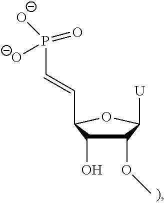

5′-Z-VP isomer (i.e., cis-vinylphosphate,

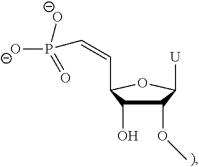

or mixtures thereof.

In one embodiment, the effector molecule (dsRNA agent) or the sciRNA agent comprises a phosphorus-containing group at the 5′-end of a sense nucleotide sequence. In one embodiment, the effector molecule (dsRNA agent) or the sciRNA agent comprises a phosphorus-containing group at the 5′-end of an antisense nucleotide sequence.

In one embodiment, the effector molecule (dsRNA agent) or the sciRNA agent comprises a 5′-P. In one embodiment, the effector molecule (dsRNA agent) or the sciRNA agent comprises a 5′-P in an antisense nucleotide sequence.

In one embodiment, the effector molecule (dsRNA agent) or the sciRNA agent comprises a 5′-PS. In one embodiment, the effector molecule (dsRNA agent) or the sciRNA agent comprises a 5′-PS in an antisense nucleotide sequence.

In one embodiment, the effector molecule (dsRNA agent) or the sciRNA agent comprises a 5′-VP. In one embodiment, the effector molecule (dsRNA agent) or the sciRNA agent comprises a 5′-VP in an antisense nucleotide sequence. In one embodiment, the effector molecule (dsRNA agent) or the sciRNA agent comprises a 5′-E-VP in an antisense nucleotide sequence. In one embodiment, the effector molecule (dsRNA agent) or the sciRNA agent comprises a 5′-Z—VP in an antisense nucleotide sequence.

In one embodiment, the effector molecule (dsRNA agent) or the sciRNA agent comprises a 5′-PS2. In one embodiment, the effector molecule (dsRNA agent) or the sciRNA (or bis-sciRNA) agent comprises a 5′-PS₂ in an antisense nucleotide sequence.

In one embodiment, the effector molecule (dsRNA agent) or the sciRNA agent comprises a 5′-PS2. In one embodiment, the effector molecule (dsRNA agent) or the sciRNA (or bis-sciRNA) agent comprises a 5′-deoxy-5′-C-malonyl in an antisense nucleotide sequence.

In some embodiments, the disclosure relates to an effector molecule (dsRNA agent) or a sciRNA agent as defined herein capable of inhibiting the expression of a target gene. The effector molecule (dsRNA agent) or the sciRNA agent comprises a sense strand and an antisense strand, each strand having 14 to 45 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand). Each of the embodiments and aspects described in this specification relating to the effector molecule (dsRNA agent) or the sciRNA represented by formula (I) can also apply to the effector molecule (dsRNA agent) or the sciRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′ end of the antisense strand.

For example, the dsRNA agent or the sciRNA (or bis-sciRNA) agent as defined herein can comprise i) a phosphorus-containing group at the 5′-end of the sense strand or antisense strand; ii) with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) a ligand, such as a ASGPR ligand (e.g., one or more GalNAc derivatives) at 5′-end or 3′-end of the sense strand or antisense strand. For instance, the ligand may be at the 3′-end of the sense strand.

In one embodiment, the effector moleculer (dsRNA agent) or the sciRNA agent further comprise a thermally destabilizing modification at position 7 counting from the 5′-end of the antisense from, at position 15 counting from the 5′-end of sense strand, position 21 counting from the 5′-end of the sense strand, or combinations thereof.

In one embodiment, the sense strand of the effector moleculer (dsRNA agent) or the sciRNA agent further comprises an endonuclease susceptible modified nucleotide at the cleavage site of the sense strand. In one example, the endonuclease susceptible modified nucleotide is at position 11 from the 5′ end of the sense strand.

In some embodiments, the effector molecule (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
   wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
   and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a four nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the effector molecules (dsRNA agent) or the sciRNA agents have a two nucleotide overhang at the 3′-end of the antisense strand, and a blunt end at the 5′-end of the antisense strand.

In another embodiment, the effector molecules (dsRNA agent) or the sciRNA agents comprise:
(a) a sense strand having:
   (i) a length of 19 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
   and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the effector molecules (dsRNA agent) or the sciRNA agents have a two nucleotide overhang at the 3′-end of the antisense strand, and a blunt end at the 5′-end of the antisense strand.

In one embodiment, the effector molecules (dsRNA agent) or the sciRNA agents comprise:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
wherein the effector molecules (dsRNA agent) or the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the effector molecules (dsRNA agent) or the sciRNA agents comprise:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the effector molecules (dsRNA agent) or the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the effector molecules (dsRNA agent) or the sciRNA agents comprise:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the effector molecules (dsRNA agent) or the sciRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

The present disclosure also includes effector molecules and multi-targeted effector molecules (or bis siRNA) sciRNA (or bis-sciRNA) molecules which are chimeric compounds. “Chimeric” compounds or “chimeras,” in the context of this disclosure, are compounds which contain two or more chemically distinct regions, each made up of at least one monomer unit, e.g., a modified or unmodified nucleotide in the case of an oligonucleotide. Chimeric compounds can be described as having a particular motif. In some embodiments, the motifs include, but are not limited to, an alternating motif, a gapped motif, a hemimer motif, a uniformly fully modified motif and a positionally modified motif. As used herein, the phrase “chemically distinct region” refers to a region in the multi-targeted molecule which is different from other regions by having a modification that is not present elsewhere in the compound or by not having a modification that is present elsewhere in the compound. A multi-targeted molecule can comprise two or more chemically distinct regions. As used herein, a region that comprises no modifications is also considered chemically distinct.

A chemically distinct region can be repeated within a multi-targeted molecule compound. Thus, a pattern of chemically distinct regions in multi-targeted molecule can be realized such that a first chemically distinct region is followed by one or more second chemically distinct regions. This sequence of chemically distinct regions can be repeated one or more times. Preferably, the sequence is repeated more than one time. For example, both strands of a double-stranded effector molecules or sciRNA (or bis-sciRNA) agent can comprise these sequences. Each chemically distinct region can actually comprise as little as single monomers, e.g., nucleotides. In some embodiments, each chemically distinct region comprises 1, 2, 3, 4, 5, 6, 7, 8, 9, 10, 11, 12, 13, 14, 15, 16, 17 or 18 monomers, e.g., nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in double-stranded effector molecule or the multi-targeted sciRNA (or bis-sciRNA) molecule have the same modification and all the even numbered nucleotides have a modification that is not present in the odd number nucleotides and vice versa.

When both strands of a double-stranded molecule comprise the alternating modification patterns, nucleotides of one strand can be complementary in position to nucleotides of the second strand which are similarly modified. In an alternative embodiment, there is a phase shift between the patterns of modifications of the first strand, respectively, relative to the pattern of similar modifications of the second strand. Preferably, the shift is such that the similarly modified nucleotides of the first strand and second strand are not in complementary position to each other.

In some embodiments, the first strand has an alternating modification pattern wherein alternating nucleotides comprise a 2′-modification, e.g., 2′-O-Methyl modification. In some embodiments, the first strand comprises an alternating 2′-O-Methyl modification and the second strand comprises an alternating 2′-fluoro modification. In other embodiments, both strands of a double-stranded oligonucleotide comprise alternating 2′-O-methyl modifications.

When both strands of a double-stranded oligonucleotide comprise alternating 2′-O-methyl modifications, such 2′-modified nucleotides can be in complementary position in the duplex region. Alternatively, such 2′-modified nucleotides may not be in complementary positions in the duplex region.

In some embodiments, an oligonucleotide present in the multi-targeted molecule comprises two chemically distinct regions, wherein each region is 1, 2, 3, 4, 5, 6, 7, 8, 9 or 10 nucleotides in length.

In other embodiments, an oligonucleotide present in the multi-targeted molecule comprises three chemically distinct regions. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to compound comprising a contiguous sequence of linked monomer subunits wherein the monomer subunits have two different types of sugar groups that alternate for essentially the entire sequence of the compound. Oligonucleotides having an alternating motif can be described by the formula: 5′-A(-L-B-L-A)n(-L-B)m-3′ where A and B are monomeric subunits that have different sugar groups, each L is an internucleoside linking group, n is from about 4 to about 12 and m is 0 or 1. This permits a compound with an alternating motif from about 9 to about 26 monomer subunits in length. This length range is not meant to be limiting as longer and shorter compounds are also amenable to the present disclosure. In some embodiments, one of A and B is a 2′-modified nucleoside as provided herein.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “type region” refers to a portion of a compound wherein the nucleosides and internucleoside linkages within the region all comprise the same type of modifications; and the nucleosides and/or the internucleoside linkages of any neighboring portions include at least one different type of modification. As used herein the term “uniformly fully modified motif” refers to an oligonucleotide comprising a contiguous sequence of linked monomer subunits that each have the same type of sugar group. In some embodiments, the uniformly fully modified motif includes a contiguous sequence of nucleosides of the present disclosure. In some embodiments, one or both of the 3′ and 5′-ends of the contiguous sequence of the nucleosides provided herein, comprise terminal groups such as one or more unmodified nucleosides.

In certain embodiments, the 5′-terminal monomer of a compound, e.g., multi-targeted sciRNA (or bis-sciRNA) molecule or an effector molecule, comprises a phosphorous moiety at the 5′-end. In certain embodiments the 5′-terminal monomer comprises a 2′-modification. In certain such embodiments, the 2′-modification of the 5′-terminal monomer is a cationic modification. In certain embodiments, the 5′-terminal monomer comprises a 5′-modification. In certain embodiments, the 5′-terminal monomer comprises a 2′-modification and a 5′-modification. In certain embodiments, the 5′-terminal monomer is a 5′-stabilizing nucleoside. In certain embodiments, the modifications of the 5′-terminal monomer stabilize the 5′-phosphate. In certain embodiments, compounds comprising modifications of the 5′-terminal monomer are resistant to exonucleases. In certain embodiments, compounds comprising modifications of the 5′-terminal monomer have improved gene expression modulating properties.

In certain embodiments, the 5′terminal monomer is attached to the rest of the compound via a modified linkage. In certain such embodiments, the 5′-terminal monomer is attached to the rest of the compound by a phosphorothioate linkage.

In certain embodiments, oligomeric compounds of the present disclosure comprise one or more regions of alternating modifications. In certain embodiments, oligomeric compounds comprise one or more regions of alternating nucleoside modifications. In certain embodiments, oligomeric compounds comprise one or more regions of alternating linkage modifications. In certain embodiments, oligomeric compounds comprise one or more regions of alternating nucleoside and linkage modifications.

In certain embodiments, oligomeric compounds of the present disclosure comprise one or more regions of alternating 2′-F modified nucleosides and 2′-OMe modified nucleosides. In certain such embodiments, such regions of alternating 2′F modified and 2′OMe modified nucleosides also comprise alternating linkages. In certain such embodiments, the linkages at the 3′ end of the 2′-F modified nucleosides are phosphorothioate linkages. In certain such embodiments, the linkages at the 3′end of the 2′OMe nucleosides are phosphodiester linkages.

In certain embodiments, such alternating regions are:
(2′-F)—(PS)-(2′-OMe)-(PO)

In certain embodiments, oligomeric compounds comprise 2, 3, 4, 5, 6, 7, 8, 9, 10, or 11 such alternatig regions. Such regions may be contiguous or may be interupted by differently modified nucleosides or linkages.

In certain embodiments, one or more alternating regions in an alternating motif include more than a single nucleoside of a type. For example, oligomeric compounds of the present disclosure may include one or more regions of any of the following nucleoside motifs:
ABA;
ABBA;
AABA;
AABBAA;
ABBABB;
AABAAB;
ABBABAABB;
ABABAA;
AABABAB;
ABABAA;
ABBAABBABABAA;
BABBAABBABABAA; or
ABABBAABBABABAA;
   wherein A is a nucleoside of a first type and B is a nucleoside of a second type. In certain embodiments, A and B are each selected from 2′-F, 2′-OMe, LNA, DNA and MOE.

In certain embodiments, A is DNA. In certain embodiments B is DNA. In some embodiments, A is 4′-CH₂O-2′-LNA. In certain embodiments, B is 4′-CH₂O-2′-LNA. In certain embodiments, A is DNA and B is 4′-CH₂O-2′-LNA. In certain embodiments A is 4′-CH₂O-2′-LNA and B is DNA.

In certain embodiments, A is 2′-OMe. In certain embodiments B is 2′-OMe. In certain embodiments, A is 2′-OMe and B is 4′-CH₂O-2′-LNA. In certain embodiments A is 4′-CH₂O-2′-LNA and B is 2′-OMe. In certain embodiments, A is 2′-OMe and B is DNA. In certain embodiments A is DNA and B is 2′-OMe.

In certain embodiments, A is (S)-cEt. In some embodiments, B is (S)-cEt. In certain embodiments, A is 2′-OMe and B is (S)-cEt. In certain embodiments A is (S)-cEt and B is 2′-OMe. In certain embodiments, A is DNA and B is (S)-cEt. In certain embodiments A is (S)-cEt and B is DNA.

In certain embodiments, A is 2′-F. In certain embodiments B is 2′-F. In certain embodiments, A is 2′-F and B is 4′-CH₂O-2′-LNA. In certain embodiments A is 4′-CH₂O-2′-LNA and B is 2′-F. In certain embodiments, A is 2′-F and B is (S)-cEt. In certain embodiments A is (S)-cEt and B is 2′-F. In certain embodiments, A is 2′-F and B is DNA. In certain embodiments A is DNA and B is 2′-F. In certain embodiments, A is 2′-OMe and B is 2′-F. In certain embodiments, A is DNA and B is 2′-OMe. In certain embodiments, A is 2′-OMe and B is DNA.

In certain embodiments, oligomeric compounds having such an alternating motif also comprise a 5′ terminal nucleoside comprising a phosphate stabilizing modification. In certain embodiments, oligomeric compounds having such an alternating motif also comprise a 5′ terminal nucleoside comprising a 2′-cationic modification. In certain embodiments, oligomeric compounds having such an alternating motif also comprise a 5′ terminal modification.

### Two-Two-Three Motifs

In certain embodiments, an oligonucleotide in the multi-targeted molecule comprises a region having a 2-2-3 motif. Such regions comprises the following motif:

5′-(E)_{w}-(A)₂-(B)ₓ-(A)₂-(C)_{y}-(A)₃-(D)_{z}
wherein: A is a first type of modified nucleoside;
B, C, D, and E are nucleosides that are differently modified than A, however, B, C, D, and E may have the same or different modifications as one another;
w and z are from 0 to 15;
x and y are from 1 to 15.

In certain embodiments, A is a 2′-OMe modified nucleoside. In certain embodiments, B, C, D, and E are all 2′-F modified nucleosides. In certain embodiments, A is a 2′-OMe modified nucleoside and B, C, D, and E are all 2′-F modified nucleosides.

In certain embodiments, the linkages of a 2-2-3 motif are all modified linkages. In certain embodiments, the linkages are all phosphorothioate linkages. In certain embodiments, the linkages at the 3′-end of each modification of the first type are phosphodiester.

In certain embodiments, Z is 0. In such embodiments, the region of three nucleosides of the first type are at the 3′-end of the oligonucleotide. In certain embodiments, such region is at the 3′-end of the oligomeric compound, with no additional groups attached to the 3′ end of the region of three nucleosides of the first type. In certain embodiments, an oligomeric compound comprising an oligonucleotide where Z is 0, may comprise a terminal group attached to the 3′-terminal nucleoside. Such terminal groups may include additional nucleosides. Such additional nucleosides are typically non-hybridizing nucleosides.

In certain embodiments, Z is 1-3. In certain embodiments, Z is 2. In certain embodiments, the nucleosides of Z are 2′-MOE nucleosides. In certain embodiments, Z represents non-hybridizing nucleosides. To avoid confussion, it is noted that such non-hybridizing nucleosides might also be described as a 3′-terminal group with Z=0.

In some embodiments, the effector molecule (dsRNA agent) or the sciRNA contains a sense strand sequence that can be represented by formula (II):

5′ nₚ-Nₐ-(XXX)ᵢ-N_{b}-YYY-N_{b}-(ZZZ)ⱼ-Nₐ-n_{q}3′ (II)
wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 1, 2, 3, 4, 5, or 6 modified nucleotides;
each nₚ and n_{q} independently represent an overhang nucleotide;
wherein N_{b} and Y do not have the same modification;
wherein XXX, YYY and ZZZ each independently represent one motif of three identical modifications on three consecutive nucleotides;
wherein the effector molecule (dsRNA agent) or the sciRNA agents have one or more lipophilic moieties conjugated to one or more positions on at least one strand; and
wherein the antisense strand of the effector molecule (dsRNA agent) or the sciRNA comprises two blocks of one, two pr three phosphorothioate internucleotide linkages separated by 1, 2, 3, 4, 5, 6, 7, 8, 9, 10, 11, 12, 13, 14, 15, 16, 17, or 18 phosphate internucleotide linkages.

Various publications described multimeric siRNA and can all be used with the dsRNA agent or the sciRNA. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, the effector molecule (dsRNA agent) or the sciRNA (or bis-sciRNA) agent contains one, two, three, four, five, six, seven, eight, nine, ten, eleven or twelve 2′-F modification(s). In one example, the effector molecule (dsRNA agent) or the sciRNA agent contains nine or ten 2′-F modifications.

The the effector molecule (dsRNA agent) or the sciRNA agent may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the effector molecule (dsRNA agent) or the sciRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paried nucleotide next to the overhang nucleotide. Preferably, these terminal three nucleotides may be at the 3′-end of the antisense strand.

### Combination Motifs

It is to be understood, that certain of the above described motifs and modifications can be combined. Since a motif may comprise only a few nucleotides, a particular oligonucleotide can comprise two or more motifs. By way of non-limiting example, in certain embodiments, an oligonucleotide in the multi-targeted molecule can have two or more nucleotide motifs selected from LNAs, phosphorthioate linkages, 2′-OMe, conjugated ligand(s).

Without limitations, the multi-targeted molecules of the present disclosure having any of the various nucleotide motifs described herein, can have also have any linkage motif. For example, in an oligonucleotide of present in the multi-targeted molecule, the first 1, 2, 3, 4 or 5 intersugar linkages at the 5′-end can be modified intrersugar linkages and the first 4, 5, 6, 7 or 8 intersugar linkages at the 3′-end can be modified intersugar linkages. The central region of such modified oligonucleotides can have intersugar linkages based on any of the other motifs described herein, for example, uniform, alternating, hemimer, gapmer, and the like. In some embodiments, an oligonucleotide of present in the multi-targeted molecule comprises a phosphorothioate linkage between the first and second monomer at the 5′-terminus, alternating phosphorothioate/phosphodiester linkages in the central region and 6, 7, or 8 phosphorothioate linkages at the 3′-terminus.

It is to be noted that the lengths of the regions defined by a nucleotide motif and that of a linkage motif need not be the same.

In some embodiments, single-stranded oligonucleotides or at least one strand of a double-stranded oligonucleotide, includes at least one of the following motifs:
5′-phosphorothioate or 5′-phosphorodithioate;
a cationic modification of nucleotides 1 and 2 on the 5′ terminal, wherein the cationic modification is at C5 position of pyrimidines and C2, C6, C8, exocyclic N2 or exocyclic N6 of purines;
at least one G-clamp nucleotide in the first two terminal nucleotides at the 5′ end and the other nucleotide having a cationic modification, wherein the cationic modification is at C5 position of pyrimidines or C2, C6, C8, exocyclic N2 or exocyclic N6 position of purines;
at least one 2′-F modified nucleotide comprising a nucleobase base modification;
at least one gem-2′-O-methyl/2′-F modified nucleotide comprising a nucleobase modification, preferably the methyl substituent is in the up configuration, e.g. in the arabinose configuration;
a 5′-PuPu-3′ dinucleotide at the 3′ terminal wherein both nucleotides comprise a modified MOE at 2′-position as described in U.S. Patent Application Publication No. 20130130378, content of which is incorporated herein by reference in its entirety,
a 5′-PuPu-3′ dinucleotide at the 5′ terminal wherein both nucleotides comprise a modified MOE at 2′-position as described in U.S. Patent Application Publication No. 20130130378;
nucleotide at the 5′ terminal having a modified MOE at 2′-position as described in U.S. Patent Application Publication No. 20130130378;
nucleotide at the 5′ terminal having a 3′-F modification;
5′ terminal nucleotide comprising a 4′-substituent;
5′ terminal nucleotide comprising a 04′ modification;
3′ terminal nucleotide comprising a 4′-substituent; and
combinations thereof.

In some embodiments, both strands of a double-stranded oligonucleotide independently comprise at least one of the above described motifs. In some other embodiments, both strands of a double-stranded oligonucleotide comprise at least one at least one of the above described motifs, which motifs can be same or different or some combination of same and different.

The above examples are provided solely to illustrate how the described motifs may be used in combination and are not intended to limit the present disclosure to the particular combinations or the particular modifications used in illustrating the combinations. Further, specific examples herein, including, but not limited to those in the above table are intended to encompass more generic embodiments. For example, column A in the above table exemplifies a region of alternating 2′-OMe and 2′-F nucleosides. Thus, that same disclosure also exemplifies a region of alternating different 2′-modifications. It also exemplifies a region of alternating 2′-O-alkyl and 2′-halogen nucleosides. It also exemplifies a region of alternating differently modified nucleosides. All of the examples throughout this specification contemplate such generic interpretation.

It is also noted that the lengths of compounds, e.g., an oligonucleotide present in the multi-targeted molecule can be easily manipulated by lengthening or shortening one or more of the described regions, without disrupting the motif.

In some embodiments, an oligonucleotide in the effector molecule or the multi-targeted molecule comprises two or more chemically distinct regions and has a structure as described in International Application No. PCT/US09/038433, filed Mar. 26, 2009, contents of which are herein incorporated in their entirety.

In some embodiments, the effector molecule (e.g., an dsRNA agent) or the sciRNA (or bis-sciRNA) agent is a ribozyme. Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes can be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

In some embodiments, the effector molecule (e.g., an dsRNA agent) or the sciRNA (or bis-sciRNA) agent is an aptamer. Aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9(1999), and Hermann and Patel, Science 287:820-5 (2000). Aptamers can be RNA or DNA based. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer can be prepared by any known method, including synthetic, recombinant, and purification methods, and can be used alone or in combination with other aptamers specific for the same target. Further, as described more fully herein, the term “aptamer” specifically includes “secondary aptamers” containing a consensus sequence derived from comparing two or more known aptamers to a given target.

Because transcription factors recognize their relatively short binding sequences, even in the absence of surrounding genomic DNA, short oligonucleotides bearing the consensus binding sequence of a specific transcription factor can be used as tools for manipulating gene expression in living cells. This strategy involves the intracellular delivery of such “decoy oligonucleotides”, which are then recognized and bound by the target factor. Accordingly, in some embodiments, the effector molecule (e.g., an dsRNA agent) or the sciRNA (or bis-sciRNA) agent is a decoy oligonucleotide.

Occupation of the transcription factor's DNA-binding site by the decoy renders the transcription factor incapable of subsequently binding to the promoter regions of target genes. Decoys can be used as therapeutic agents, either to inhibit the expression of genes that are activated by a transcription factor, or to up-regulate genes that are suppressed by the binding of a transcription factor. Examples of the utilization of decoy oligonucleotides can be found in Mann et al., J. Clin. Invest., 2000, 106: 1071-1075, which is expressly incorporated by reference herein, in its entirety.

In some embodiments, the effector molecule (e.g., an dsRNA agent) or the sciRNA (or bis-sciRNA) agent is a U1 adaptor. U1 adaptors inhibit polyA sites and are bifunctional oligonucleotides with a target domain complementary to a site in the target gene's terminal exon and a ‘U1 domain’ that binds to the U1 smaller nuclear RNA component of the U1 snRNP. See for example, Int. Pat. App. Pub. No. WO2008/121963 and Goraczniak, et al., 2008, Nature Biotechnology, 27(3), 257-263, each of which is expressly incorporated by reference herein, in its entirety. U1 snRNP is a ribonucleoprotein complex that functions primarily to direct early steps in spliceosome formation by binding to the pre-mRNA exon-intron boundary, Brown and Simpson, 1998, Annu Rev Plant Physiol Plant MoI Biol 49:77-95.

In some embodiments, the U1 adaptor comprises at least one annealing domain (targeting domain) linked to at least one effector domain (U1 domain), wherein the annealing domain hybridizes to a target gene sequence and the effector domain hybridizes to the U1 snRNA of U1 snRNP. In some embodiments, the U1 adaptor comprises one annealing domain. In some embodiments, the U1 adaptor comprises one effector domain.

Without wishing to be bound by theory, the annealing domain will typically be from about 10 to about 50 nucleotides in length, more typically from about 10 to about 30 nucleotides or about 10 to about 20 nucleotides. In some preferred embodiments, the annealing domain is 10, 11, 12, 13, 14, 15, 16, 17, 18, 19, 20, or 21 nucleotides in length. The annealing domain may be at least 75%, at least 80%, at least 85%, at least 90%, at least 95%, at least 97%, or, more preferably, 100% complementary to the target gene. In some embodiments, the annealing domain hybridizes with a target site within the 3′ terminal exon of a pre-mRNA, which includes the terminal coding region and the 3′UTR and polyadenylation signal sequences (e.g., through the polyadenylation site). In another embodiment, the target sequence is within about 500 basepair, about 250 basepair, about 100 basepair, or about 50 basepair of the poly (A) signal sequence of the pre-mRNA. In some embodiments, the annealing domain comprises 1, 2, 3, or 4, mismatches with the target gene sequence.

The effector domain may be from about 8 nucleotides to about 30 nucleotides, from about 10 nucleotides to about 20 nucleotides, or from about 10 to about 15 nucleotides in length. The Ul domain can hybridize with Ul snRNA, particularly the 5′-end and more specifically nucleotides 2-11. In another embodiment, the Ul domain is perfectly complementary to nucleotides 2-11 of endogenous Ul snRNA. In some embodiments, the U1 domain comprises a nucleotide sequence selected from the group consisting of 5′-GCCAGGUAAGUAU-3′, 5′-CCAGGUAAGUAU-3′, 5′-CAGGUAAGUAU-3′, 5′-CAGGUAAGU-3′, 5′-CAGGUAAG-3′ and 5′-CAGGUAA-3′. In some embodiments, the U1 domain comprises a nucleotide sequence 5′-CAGGUAAGUA-3′. Without wishing to be bound by theory, increasing the length of the Ul domain to include basepairing into stem 1 and/or basepairing to position 1 of Ul snRNA improves the Ul adaptor's affinity to Ul snRNP.

The annealing and effector domains of the Ul adaptor can be linked such that the effector domain is at the 5′ end and/or 3′ end of the annealing domain. The two domains can be linked by such that the 3′ end of one domain is linked to 5′ end of the other domain, or 3′ end of one domain is linked to 3′ end of the other domain, or 5′ end of one domain is linked to 5′ end of the other domain. The annealing and effector domains can be linked directly to each other or by a nucleotide based or non-nucleotide based linker. When the linker is nucleotide based, the linker can comprise 1, 2, 3, 4, 5, 6, 7, 8, 9, 10, up to 15, up to 20, or up to 25 nucleotides.

In some embodiments, the linker between the annealing domain and the effector domain is a cleavable linker described herein. In some embodiments, the linker between the annealing domain and the effector domain is mutlivalent, e.g., trivalent, tetravalent or pentavalent. Without wishing to be bound by theory, a multivalent linker can be used to link together a single annealing domain with a plurality of adaptor domains.

It is to be understood that the U1 adaptor can comprise any oligonucleotide modification described herein. Exemplary modifications for U1 adaptors include those that increase annealing affinity, specificity, bioavailability in the cell and organism, cellular and/or nuclear transport, stability, and/or resistance to degradation.

In some embodiments, the effector molecule (e.g., an dsRNA agent) or the sciRNA (or bis-sciRNA) agent is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′overhang.

In some embodiments, the effector molecule (e.g., an dsRNA agent) or the sciRNA (or bis-sciRNA) agent is an antimir. In some embodiments, the effector molecule (e.g., an dsRNA agent) or the sciRNA (or bis-sciRNA) agent comprises at least two antimirs covalently linked to each other via a nucleotide-based or non-nucleotide-based linker, for example, a linker described in the disclosure, or non-covlantly linked to each other. The terms “antimir” “microRNA inhibitor” or “miR inhibitor” are synonymous and refer to oligonucleotides or modified oligonucleotides that interfere with the activity of specific miRNAs. Inhibitors can adopt a variety of configurations including single stranded, double stranded (RNA/RNA or RNA/DNA duplexes), and hairpin designs, in general, microRNA inhibitors comprise one or more sequences or portions of sequences that are complementary or partially complementary with the mature strand (or strands) of the miRNA to be targeted, in addition, the miRNA inhibitor can also comprise additional sequences located 5′ and 3′ to the sequence that is the reverse complement of the mature miRNA. The additional sequences can be the reverse complements of the sequences that are adjacent to the mature miRNA in the pri-miRNA from which the mature miRNA is derived, or the additional sequences can be arbitrary sequences (having a mixture of A, G, C, U, or dT). In some embodiments, one or both of the additional sequences are arbitrary sequences capable of forming hairpins. Thus, in some embodiments, the sequence that is the reverse complement of the miRNA is flanked on the 5′ side and on the 3′ side by hairpin structures. MicroRNA inhibitors, when double stranded, can include mismatches between nucleotides on opposite strands. Furthermore, microRNA inhibitors can be linked to conjugate moieties in order to facilitate uptake of the inhibitor into a cell.

MicroRNA inhibitors, including hairpin miRNA inhibitors, are described in detail in Vermeulen et al., “Double-Stranded Regions Are Essential Design Components Of Potent Inhibitors of RISC Function,” RNA 13: 723-730 (2007) and in WO2007/095387 and WO 2008/036825 each of which is incorporated herein by reference in its entirety. A person of ordinary skill in the art can select a sequence from the database for a desired miRNA and design an inhibitor useful for the methods disclosed herein.

In some embodiments, the effector molecule (e.g., an dsRNA agent) or the sciRNA (or bis-sciRNA) agent is an antagomir. In some embodiments, the effector molecule (e.g., an dsRNA agent) or the sciRNA (or bis-sciRNA) agent comprises at least two antagomirs covalently linked to each other via a nucleotide-based or non-nucleotide-based linker, for example, a linker described in the disclosure, or non-covlantly linked to each other. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In a preferred embodiment, antagomir comprises a 2′-O-methyl modification at all nucleotides, a cholesterol moiety at 3′-end, two phsophorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Recent studies have found that dsRNA can also activate gene expression, a mechanism that has been termed “small RNA-induced gene activation” or RNAa (activating RNA). See, for example, Li, L. C. et al. Proc Natl Acad Sci USA. (2006), 103(46):17337-42 and Li L. C. (2008). “Small RNA-Mediated Gene Activation”. RNA and the Regulation of Gene Expression: A Hidden Layer of Complexity. Caister Academic Press. ISBN 978-1-904455-25-7. It has been shown that dsRNAs targeting gene promoters induce potent transcriptional activation of associated genes. Endogenous miRNA that cause RNAa has also been found in humans. Check E. Nature (2007). 448 (7156): 855-858.

Another surprising observation is that gene activation by RNAa is long-lasting. Induction of gene expression has been seen to last for over ten days. The prolonged effect of RNAa could be attributed to epigenetic changes at dsRNA target sites. In some embodiments, the RNA activator can increase the expression of a gene. In some embodiments, increased gene expression inhibits viability, growth development, and/or reproduction.

Accordingly, in some embodiments, the effector molecule (e.g., an dsRNA agent) or the sciRNA (or bis-sciRNA) agent is activating RNA. In some embodiments, the effector molecule (e.g., an dsRNA agent) or the sciRNA (or bis-sciRNA) agent comprises at least two activating RNAs scovalently linked to each other via a nucleotide-based or non-nucleotide-based linker, for example, a linker described in the disclosure, or non-covlantly linked to each other.

Accordingly, in some embodiments, the effector molecule (e.g., an dsRNA agent) or the sciRNA (or bis-sciRNA) agent is a triplex forming oligonucotide (TFO). In some embodiments, the effector molecule (e.g., an dsRNA agent) or the sciRNA (or bis-sciRNA) agent comprises at least two TFOs covalently linked to each other via a nucleotide-based or non-nucleotide-based linker, for example, a linker described in the disclosure, or non-covlantly linked to each other. Recent studies have shown that triplex forming oligonucleotides can be designed which can recognize and bind to polypurine/polypyrimidine regions in double-stranded helical DNA in a sequence-specific manner. These recognition rules are outline by Maher III, L. J., et al., Science (1989) vol. 245, pp 725-730; Moser, H. E., et al., Science (1987) vol. 238, pp 645-630; Beal, P. A., et al., Science (1992) vol. 251, pp 1360-1363; Conney, M., et al., Science (1988) vol. 241, pp 456-459 and Hogan, M. E., et al., EP Publication 375408. Modification of the oligonucleotides, such as the introduction of intercalators and intersugar linkage substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 1 12:487-94). In general, the triplex-forming oligonucleotide has the sequence correspondence:
oligo 3′-A G G T
duplex 5′-A G C T
duplex 3′-T C G A

However, it has been shown that the A-AT and G-GC triplets have the greatest triple helical stability (Reither and Jeltsch, BMC Biochem, 2002, Septl2, Epub). The same authors have demonstrated that TFOs designed according to the A-AT and G-GC rule do not form non-specific triplexes, indicating that the triplex formation is indeed sequence specific.

Thus for any given sequence a triplex forming sequence can be devised. Triplex-forming oligonucleotides preferably are at least 15, more preferably 25, still more preferably 30 or more nucleotides in length, up to 50 or 100 nucleotides.

Formation of the triple helical structure with the target DNA induces steric and functional changes, blocking transcription initiation and elongation, allowing the introduction of desired sequence changes in the endogenous DNA and resulting in the specific down-regulation of gene expression. Examples of such suppression of gene expression in cells treated with TFOs include knockout of episomal supFGl and endogenous HPRT genes in mammalian cells (Vasquez et al., Nucl Acids Res. 1999; 27: 1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-I gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both down-regulation and up-regulation of expression of endogenous genes (Seidman and Glazer, J Clin Invest 2003; 112:487-94). Detailed description of the design, synthesis and administration of effective TFOs can be found in U.S. Pat. App. Nos. 2003 017068 and 2003 0096980 to Froehler et al, and 2002 0128218 and 2002 0123476 to Emanuele et al, and U.S. Pat. No. 5,721,138 to Lawn, contents of which are herein incorporated in their entireties.

It is contemplated that TFOs can also be designed to combine one or more dsRNA effector molecule (e.g., a siRNA) or a sciRNA (or bis-sciRNA) agent via linkage to an inhibitory single-stranded oligonucleotide or a single-stranded small interfering RNA (ss-siRNA).

### Nucleic Acid Modifications

In some embodiments, the effector molecule (e.g., an dsRNA agent) or the sciRNA (or bis-sciRNA) agent comprises at least one nucleic acid modification described herein. For example, at least one modification selected from the group consisting of modified internucleoside linkage, modified nucleobase, modified sugar, and any combinations thereof. Without limitations, such a modification can be present anywhere in the effector molecule (e.g., an dsRNA agent) or the sciRNA (or bis-sciRNA) agent. For example, the modification can be present in one of the RNA molecules; alternatively, the modification can be present in a linker connecting two effector molecules of the multi-targeted molecule or a linker connecting the sequences of the multi-targeted bis-sciRNA molecule.

### Nucleic Acid Modifications (Nucleobases)

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as a 7-deaza purine, a 5-methyl cytosine, or a G-clamp. In certain embodiments, nucleobase mimetic include more complicated structures, such as a tricyclic phenoxazine nucleobase mimetic. Methods for preparation of the above noted modified nucleobases are well known to those skilled in the art.

### Nucleic Acid Modifications (Sugar)

The effector molecule (e.g., an dsRNA agent) or the sciRNA (or bis-sciRNA) agent can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ—, —[C(R1)(R2)]ₙ—O—, —[C(R1)(R2)]ₙ—N(R1)-, —[C(R1)(R2)]ₙ-N(R1)-O—, —[C(R1R2)]ₙ-O—N(R1)-, —C(R1)=C(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2)-N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L-methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)2-O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

The effector molecule (e.g., a dsRNA agent) or the sciRNA (or bis-sciRNA) agent disclosed herein can also include abasic sugars, i.e., a sugar which lack a nucleobase at C-1′ or has other chemical groups in place of a nucleobase at C1′. See, for example, U.S. Pat. No. 5,998,203, content of which is herein incorporated in its entirety. These abasic sugars can also be further containing modifications at one or more of the constituent sugar atoms. The effector molecule (e.g., a dsRNA agent) or the sciRNA (or bis-sciRNA) agent can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

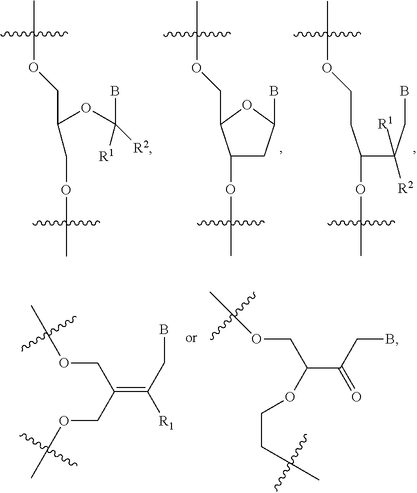

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

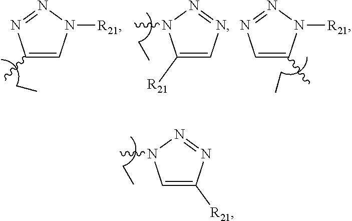

NR₂₁R₃₁, CONR₂₁R₃₁, CON(H)NR₂₁R₃₁, ONR₂₁R₃₁, CON(H)N═CR₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═CR₄₁R₅₁, ON═CR₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicyclic nucleoside having the formula:

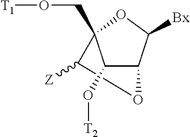

wherein:
Bx is a heterocyclic base moiety;
T₁ is H or a hydroxyl protecting group;
T₂ is H, a hydroxyl protecting group or a reactive phosphorus group;
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain embodiments, the effector molecule (e.g., a dsRNA agent) or the sciRNA (or bis-sciRNA) agent comprises at least one monomer of the formula:

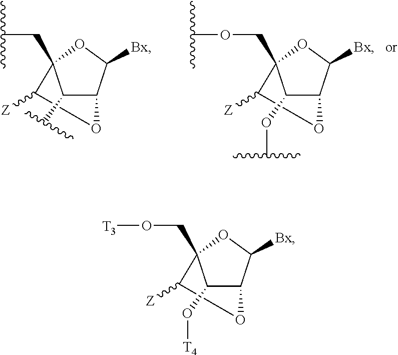

wherein
Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below:

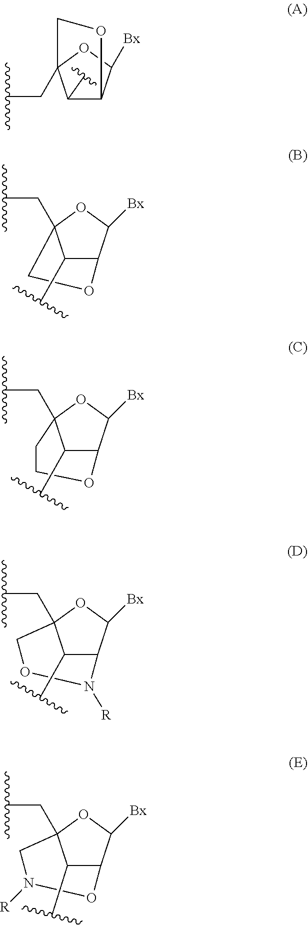

In certain embodiments, the effector molecule (e.g., an dsRNA agent) or the sciRNA (or bis-sciRNA) agent comprises at least two regions of at least two contiguous monomers of the above formula. In certain embodiments, the effector molecule (e.g., an dsRNA agent) or the sciRNA (or bis-sciRNA) agent comprises a gapped motif. In certain embodiments, the effector molecule (e.g., an dsRNA agent) or the sciRNA (or bis-sciRNA) agent comprises at least one region of from about 8 to about 14 contiguous β-D-2′-deoxyribofuranosyl nucleosides. In certain embodiments, the effector molecule (e.g., an dsRNA agent) or the sciRNA (or bis-sciRNA) agent comprises at least one region of from about 9 to about 12 contiguous β-D-2′-deoxyribofuranosyl nucleosides.

In certain embodiments, the effector molecule (e.g., an dsRNA agent) or the sciRNA (or bis-sciRNA) agent comprises at least one (e.g., 1, 2, 3, 4, 5, 6, 7, 8, 9, 10, 11, 12, 13, 14, 15 or more) comprises at least one (S)-cEt monomer of the formula:

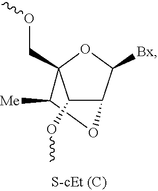

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### Nucleic Acid Modifications (Intersugar Linkage)

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words, a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (e.g., amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the effector molecule (e.g., an dsRNA agent) or the sciRNA (or bis-sciRNA) agent comprises at least one (e.g., 1, 2, 3, 4, 5, 6, 7, 8, 9, 10, 11, 12, 13, 14, 15 or more and upto including all) modified or nonphosphodiester linkages. In some embodiments, the effector molecule (e.g., an dsRNA agent) or the sciRNA (or bis-sciRNA) agent comprises at least one (e.g., 1, 2, 3, 4, 5, 6, 7, 8, 9, 10, 11, 12, 13, 14, 15 or more and upto including all) phosphorothioate linkages.

The effector molecule (e.g., an dsRNA agent) or the sciRNA (or bis-sciRNA) agent can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). It can be desirable, in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The effector molecule (e.g., an dsRNA agent) or the sciRNA (or bis-sciRNA) agent described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the effector molecule (e.g., an dsRNA agent) or the sciRNA (or bis-sciRNA) agent provided herein are all such possible isomers, as well as their racemic and optically pure forms.

### Nucleic Acid Modifications (Terminal Modifications)

Ends of a sense or antisense nucleotide sequence of the effector molecule (e.g., an dsRNA agent) or the sciRNA (or bis-sciRNA) agent can be modified. Such modifications can be at one end or both ends of the nucleotide sequence. For example, the 3′ and/or 5′ ends can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end of a sequence with phosphate or phosphate analogs. In certain embodiments, the 5′end of sequence is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

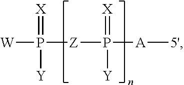

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)₂(X)P—O[—(CH₂)ₐ—P(X)(OH)—O]_{b}-5′, ((HO)₂(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### Thermally Destabilizing Modifications

The compounds of the invention, such as the effector molecule (e.g., iRNAs) or sciRNA (or bis-sciRNA) agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nuceltic acid (GNA).

Exemplified abasic modifications are:

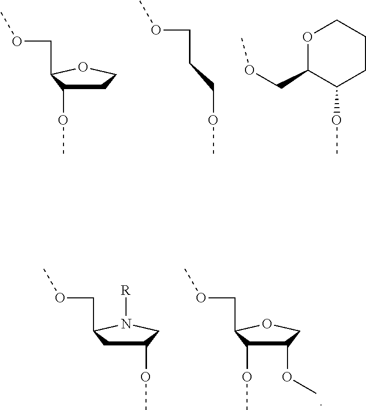

Exemplified sugar modifications are:

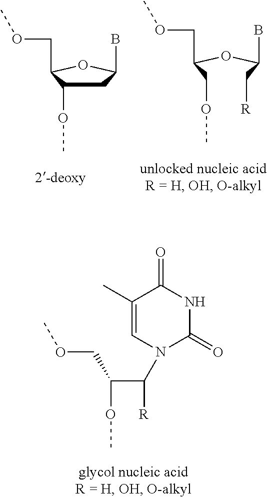

The term “acyclic nucleotide” refers to any nucleotide having an acyclic ribose sugar, for example, where any of bonds between the ribose carbons (e.g., C1′-C2′, C2′-C3′, C3′-C4′, C4′-04′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

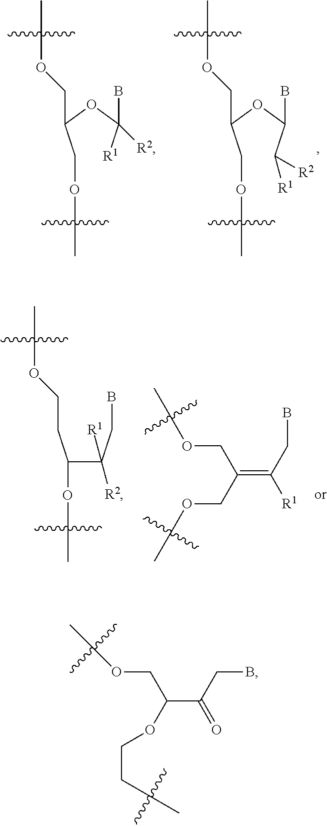

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

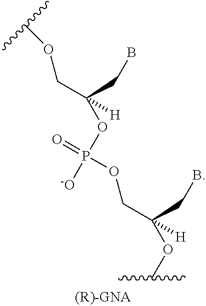

The thermally destabilizing modification can be mismatches (i.e., noncomplementary base pairs) between the thermally destabilizing nucleotide and the opposing nucleotide in the opposite strand within the effector molecule (e.g., an dsRNA agent) or the sciRNA (or bis-sciRNA) duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the effector molecule (e.g., an dsRNA agent) or the sciRNA (or bis-sciRNA) agent contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

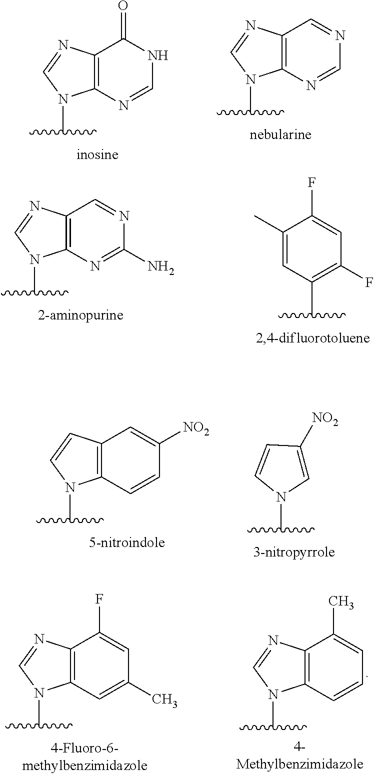

Exemplary phosphate modifications known to decrease the thermal stability of dsRNA duplexes compared to natural phosphodiester linkages are:

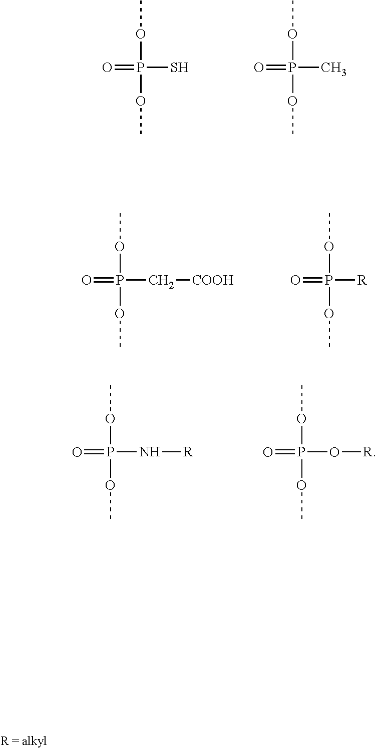

In some embodiments, the effector molecule (e.g., an dsRNA agent) or the sciRNA (or bis-sciRNA) agent can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the effector molecule (e.g., an dsRNA agent) or the sciRNA (or bis-sciRNA) agent can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In some embodiments, at least one strand of the effector molecule (e.g., an dsRNA agent) or the sciRNA (or bis-sciRNA) agent disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=methoxymethyl (MeOCH2-), ethoxymethyl, etc., e.g. RP(OH)(O)—O-5′-).

### Target Genes

Without limitations, target genes for the effector molecules (dsRNA agent) or sciRNAs (or bis-sciRNAs) include, but are not limited to genes promoting unwanted cell proliferation, growth factor gene, growth factor receptor gene, genes expressing kinases, an adaptor protein gene, a gene encoding a G protein super family molecule, a gene encoding a transcription factor, a gene which mediates angiogenesis, a viral gene, a gene required for viral replication, a cellular gene which mediates viral function, a gene of a bacterial pathogen, a gene of an amoebic pathogen, a gene of a parasitic pathogen, a gene of a fungal pathogen, a gene which mediates an unwanted immune response, a gene which mediates the processing of pain, a gene which mediates a neurological disease, an allene gene found in cells characterized by loss of heterozygosity, or one allege gene of a polymorphic gene.

Specific exemplary target genes for the effector molecules (dsRNA agent) or the sciRNAs (or bis-sciRNAs) include, but are not limited to, PCSK-9, ApoC3, AT3, AGT, ALAS1, TMPR, HAO1, AGT, C5, CCR-5, PDGF beta gene; Erb-B gene, Src gene; CRK gene; GRB2 gene; RAS gene; MEKK gene; JNK gene; RAF gene; Erk1/2 gene; PCNA(p21) gene; MYB gene; c-MYC gene; JUN gene; FOS gene; BCL-2 gene; Cyclin D gene; VEGF gene; EGFR gene; Cyclin A gene; Cyclin E gene; WNT-1 gene; beta-catenin gene; c-MET gene; PKC gene; NFKB gene; STAT3 gene; survivin gene; Her2/Neu gene; topoisomerase I gene; topoisomerase II alpha gene; p73 gene; p21(WAF1/CIP1) gene, p27(KIP1) gene; PPM1D gene; caveolin I gene; MIB I gene; MTAI gene; M68 gene; tumor suppressor genes; p53 gene; DN-p63 gene; pRb tumor suppressor gene; APC1 tumor suppressor gene; BRCA1 tumor suppressor gene; PTEN tumor suppressor gene; MLL fusion genes, e.g., MLL-AF9, BCR/ABL fusion gene; TEL/AML1 fusion gene; EWS/FLIl fusion gene; TLS/FUS1 fusion gene; PAX3/FKHR fusion gene; AML1/ETO fusion gene; alpha v-integrin gene; Flt-1 receptor gene; tubulin gene; Human Papilloma Virus gene, a gene required for Human Papilloma Virus replication, Human Immunodeficiency Virus gene, a gene required for Human Immunodeficiency Virus replication, Hepatitis A Virus gene, a gene required for Hepatitis A Virus replication, Hepatitis B Virus gene, a gene required for Hepatitis B Virus replication, Hepatitis C Virus gene, a gene required for Hepatitis C Virus replication, Hepatitis D Virus gene, a gene required for Hepatitis D Virus replication, Hepatitis E Virus gene, a gene required for Hepatitis E Virus replication, Hepatitis F Virus gene, a gene required for Hepatitis F Virus replication, Hepatitis G Virus gene, a gene required for Hepatitis G Virus replication, Hepatitis H Virus gene, a gene required for Hepatitis H Virus replication, Respiratory Syncytial Virus gene, a gene that is required for Respiratory Syncytial Virus replication, Herpes Simplex Virus gene, a gene that is required for Herpes Simplex Virus replication, herpes Cytomegalovirus gene, a gene that is required for herpes Cytomegalovirus replication, herpes Epstein Barr Virus gene, a gene that is required for herpes Epstein Barr Virus replication, Kaposi's Sarcoma-associated Herpes Virus gene, a gene that is required for Kaposi's Sarcoma-associated Herpes Virus replication, JC Virus gene, human gene that is required for JC Virus replication, myxovirus gene, a gene that is required for myxovirus gene replication, rhinovirus gene, a gene that is required for rhinovirus replication, coronavirus gene, a gene that is required for coronavirus replication, West Nile Virus gene, a gene that is required for West Nile Virus replication, St. Louis Encephalitis gene, a gene that is required for St. Louis Encephalitis replication, Tick-borne encephalitis virus gene, a gene that is required for Tick-borne encephalitis virus replication, Murray Valley encephalitis virus gene, a gene that is required for Murray Valley encephalitis virus replication, dengue virus gene, a gene that is required for dengue virus gene replication, Simian Virus 40 gene, a gene that is required for Simian Virus 40 replication, Human T Cell Lymphotropic Virus gene, a gene that is required for Human T Cell Lymphotropic Virus replication, Moloney-Murine Leukemia Virus gene, a gene that is required for Moloney-Murine Leukemia Virus replication, encephalomyocarditis virus gene, a gene that is required for encephalomyocarditis virus replication, measles virus gene, a gene that is required for measles virus replication, Vericella zoster virus gene, a gene that is required for Vericella zoster virus replication, adenovirus gene, a gene that is required for adenovirus replication, yellow fever virus gene, a gene that is required for yellow fever virus replication, poliovirus gene, a gene that is required for poliovirus replication, poxvirus gene, a gene that is required for poxvirus replication, plasmodium gene, a gene that is required for plasmodium gene replication, Mycobacterium ulcerans gene, a gene that is required for Mycobacterium ulcerans replication, Mycobacterium tuberculosis gene, a gene that is required for Mycobacterium tuberculosis replication, Mycobacterium leprae gene, a gene that is required for Mycobacterium leprae replication, Staphylococcus aureus gene, a gene that is required for Staphylococcus aureus replication, Streptococcus pneumoniae gene, a gene that is required for Streptococcus pneumoniae replication, Streptococcus pyogenes gene, a gene that is required for Streptococcus pyogenes replication, Chlamydia pneumoniae gene, a gene that is required for Chlamydia pneumoniae replication, Mycoplasma pneumoniae gene, a gene that is required for Mycoplasma pneumoniae replication, an integrin gene, a selectin gene, complement system gene, chemokine gene, chemokine receptor gene, GCSF gene, Gro1 gene, Gro2 gene, Gro3 gene, PF4 gene, MIG gene, Pro-Platelet Basic Protein gene, MIP-1I gene, MIP-1J gene, RANTES gene, MCP-1 gene, MCP-2 gene, MCP-3 gene, CMBKR1 gene, CMBKR2 gene, CMBKR3 gene, CMBKR5v, AIF-1 gene, I-309 gene, a gene to a component of an ion channel, a gene to a neurotransmitter receptor, a gene to a neurotransmitter ligand, amyloid-family gene, presenilin gene, HD gene, DRPLA gene, SCA1 gene, SCA2 gene, MJD1 gene, CACNLIA4 gene, SCA7 gene, SCA8 gene, allele gene found in loss of heterozygosity (LOH) cells, one allele gene of a polymorphic gene and combinations thereof.

The loss of heterozygosity (LOH) can result in hemizygosity for sequence, e.g., genes, in the area of LOH. This can result in a significant genetic difference between normal and disease-state cells, e.g., cancer cells, and provides a useful difference between normal and disease-state cells, e.g., cancer cells. This difference can arise because a gene or other sequence is heterozygous in duploid cells but is hemizygous in cells having LOH. The regions of LOH will often include a gene, the loss of which promotes unwanted proliferation, e.g., a tumor suppressor gene, and other sequences including, e.g., other genes, in some cases a gene which is essential for normal function, e.g., growth. Methods of the invention rely, in part, on the specific modulation of one allele of an essential gene with a composition of the invention.

In certain embodiments, the invention provides a multi-targeted molecule (e.g., effector molecules such as bis siRNA or sciRNA (or bis-sciRNA) agent) that modulates a micro-RNA.

In some embodiments, the invention provides a multi-targeted molecule (e.g., effector molecules such as bis siRNA or sciRNA (or bis-sciRNA) agent) for extrahepatic delivery, and target a CNS gene or ocular gene.

In some embodiments, provided herein is a multi-targeted molecule (e.g., effector molecules such as bis siRNA or sciRNA (or bis-sciRNA)) that targets APP for Early Onset Familial Alzheimer Disease, ATXN2 for Spinocerebellar Ataxia 2 and ALS, and C9orf72 for Amyotrophic Lateral Sclerosis and Frontotemporal Dementia.

In some embodiments, provided herein is a multi-targeted molecule (e.g., effector molecules such as bis siRNA or sciRNA (or bis-sciRNA) agent) that targets TARDBP for ALS, MAPT (Tau) for Frontotemporal Dementia, and HTT for Huntington Disease.

In some embodiments, provided herein is a multi-targeted molecule (e.g., effector molecules such as bis siRNA or sciRNA (or bis-sciRNA) agent) that targets SNCA for Parkinson Disease, FUS for ALS, ATXN3 for Spinocerebellar Ataxia 3, ATXN1 for SCA1, genes for SCA7 and SCA8, ATN1 for DRPLA, MeCP2 for XLMR, PRNP for Prion Diseases, recessive CNS disorders: Lafora Disease, DMPK for DM1 (CNS and Skeletal Muscle), and TTR for hATTR (CNS, ocular and systemic).

Spinocerebellar ataxia is an inherited brain-function disorder. Dominantly inherited forms of spinocerebellar ataxias, such as SCA1-8, are devastating disorders with no disease-modifying therapy. Exemplary targets include SCA2, SCA3, and SCA1.

Additional examples of CNS gene and ocular gene can be found in WO 2019/217459, which is incorporated herein by reference in its entirety.

Evaluation of Candidate iRNAs

One can evaluate a candidate iRNA agent, e.g., a modified RNA, for a selected property by exposing the agent or modified molecule and a control molecule to the appropriate conditions and evaluating for the presence of the selected property. For example, resistance to a degradent can be evaluated as follows. A candidate modified RNA (and a control molecule, usually the unmodified form) can be exposed to degradative conditions, e.g., exposed to a milieu, which includes a degradative agent, e.g., a nuclease. E.g., one can use a biological sample, e.g., one that is similar to a milieu, which might be encountered, in therapeutic use, e.g., blood or a cellular fraction, e.g., a cell-free homogenate or disrupted cells. The candidate and control could then be evaluated for resistance to degradation by any of a number of approaches. For example, the candidate and control could be labeled prior to exposure, with, e.g., a radioactive or enzymatic label, or a fluorescent label, such as Cy3 or Cy5. Control and modified RNA's can be incubated with the degradative agent, and optionally a control, e.g., an inactivated, e.g., heat inactivated, degradative agent. A physical parameter, e.g., size, of the modified and control molecules are then determined. They can be determined by a physical method, e.g., by polyacrylamide gel electrophoresis or a sizing column, to assess whether the molecule has maintained its original length, or assessed functionally. Alternatively, Northern blot analysis can be used to assay the length of an unlabeled modified molecule.

A functional assay can also be used to evaluate the candidate agent. A functional assay can be applied initially or after an earlier non-functional assay, (e.g., assay for resistance to degradation) to determine if the modification alters the ability of the molecule to silence gene expression. For example, a cell, e.g., a mammalian cell, such as a mouse or human cell, can be co-transfected with a plasmid expressing a fluorescent protein, e.g., GFP, and a candidate RNA agent homologous to the transcript encoding the fluorescent protein (see, e.g., WO 00/44914). For example, a modified dsiRNA homologous to the GFP mRNA can be assayed for the ability to inhibit GFP expression by monitoring for a decrease in cell fluorescence, as compared to a control cell, in which the transfection did not include the candidate dsiRNA, e.g., controls with no agent added and/or controls with a non-modified RNA added. Efficacy of the candidate agent on gene expression can be assessed by comparing cell fluorescence in the presence of the modified and unmodified dsiRNA compounds.

In an alternative functional assay, a candidate dsiRNA compound homologous to an endogenous mouse gene, for example, a maternally expressed gene, such as c-mos, can be injected into an immature mouse oocyte to assess the ability of the agent to inhibit gene expression in vivo (see, e.g., WO 01/36646). A phenotype of the oocyte, e.g., the ability to maintain arrest in metaphase II, can be monitored as an indicator that the agent is inhibiting expression. For example, cleavage of c-mos mRNA by a dsiRNA compound would cause the oocyte to exit metaphase arrest and initiate parthenogenetic development (Colledge et al. Nature 370: 65-68, 1994; Hashimoto et al. Nature, 370:68-71, 1994). The effect of the modified agent on target RNA levels can be verified by Northern blot to assay for a decrease in the level of target mRNA, or by Western blot to assay for a decrease in the level of target protein, as compared to a negative control. Controls can include cells in which with no agent is added and/or cells in which a non-modified RNA is added.

### Physiological Effects

The multi-targeted molecules (e.g., effector molecules such as bis siRNA or sciRNA (or bis-sciRNA) compounds) described herein can be designed such that determining therapeutic toxicity is made easier by the complementarity of the siRNA with both a human and a non-human animal sequence. By these methods, an siRNA can consist of a sequence that is fully complementary to a nucleic acid sequence from a human and a nucleic acid sequence from at least one non-human animal, e.g., a non-human mammal, such as a rodent, ruminant or primate. For example, the non-human mammal can be a mouse, rat, dog, pig, goat, sheep, cow, monkey, Pan paniscus, Pan troglodytes, Macaca mulatto, or Cynomolgus monkey. The sequence of the siRNA compound could be complementary to sequences within homologous genes, e.g., oncogenes or tumor suppressor genes, of the non-human mammal and the human. By determining the toxicity of the siRNA compound in the non-human mammal, one can extrapolate the toxicity of the siRNA compound in a human. For a more strenuous toxicity test, the siRNA can be complementary to a human and more than one, e.g., two or three or more, non-human animals.

The methods described herein can be used to correlate any physiological effect of a multi-targeted molecule (e.g., effector molecules such as bis siRNA or sciRNA (or bis-sciRNA) compound) on a human, e.g., any unwanted effect, such as a toxic effect, or any positive, or desired effect.

Increasing Cellular Uptake of siRNAs

Described herein are various multi-targeted molecule (e.g., effector molecules such as bis siRNA or sciRNA (or bis-sciRNA)) compositions that contain covalently attached conjugates that increase cellular uptake and/or intracellular targeting of the effector molecules or sciRNA (or bis-sciRNA)s.

Additionally provided are methods of the invention that include administering a multi-targeted molecule (e.g., effector molecules such as bis siRNA or sciRNA (or bis-sciRNA) compound) and a drug that affects the uptake of the multi-targeted molecule (e.g., effector molecules such as bis siRNA or sciRNA (or bis-sciRNA)) into the cell. The drug can be administered before, after, or at the same time that the multi-targeted molecule (e.g., effector molecules such as bis siRNA or sciRNA (or bis-sciRNA) compound) is administered. The drug can be covalently or non-covalently linked to the multi-targeted molecule (e.g., effector molecules such as bis siRNA or sciRNA (or bis-sciRNA) compound). The drug can be, for example, a lipopolysaccharide, an activator of p38 MAP kinase, or an activator of NF-κB. The drug can have a transient effect on the cell. The drug can increase the uptake of the multi-targeted molecule (e.g., effector molecules such as bis siRNA or sciRNA (or bis-sciRNA) compound) into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The drug can also increase the uptake of the multi-targeted molecule (e.g., effector molecules such as bis siRNA or sciRNA (or bis-sciRNA) compound) into a given cell or tissue by activating an inflammatory response, for example. Exemplary drugs that would have such an effect include tumor necrosis factor alpha (TNFalpha), interleukin-1 beta, a CpG motif, gamma interferon or more generally an agent that activates a toll-like receptor.

Effector Molecule (dsRNA) or sciRNA (or Bis-sciRNA) Production

An effector molecule (dsRNA) or sciRNA (or bis-sciRNA) can be produced, e.g., in bulk, by a variety of methods. Exemplary methods include: organic synthesis and RNA cleavage, e.g., in vitro cleavage.

Organic Synthesis. An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

dsiRNA Cleavage. siRNAs can also be made by cleaving a larger siRNA. The cleavage can be mediated in vitro or in vivo. For example, to produce iRNAs by cleavage in vitro, the following method can be used:

In vitro transcription. dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes.

In Vitro Cleavage. In one embodiment, RNA generated by this method is carefully purified to remove and siRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9. and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

### Making Double-Stranded RNA Agents Conjugated to a Ligand

In some embodiments, a ligand can be conjugated to the multi-targeted molecule (e.g., the effector molecules such as bis siRNA or the sciRNA agent) via a nucleobase, sugar moiety, internucleosidic linkage, or a carrier, as described herein.

Detailed methods for conjugating a ligand to an siRNA agent (such as a lipophilic moiety or a carbohydrate-based ligand) can be found in WO 2019/217459, WO 2009/082607, and WO 2009/073809, all of which are incorporated herein by reference in their entirety.

### Pharmaceutical Compostions

In one aspect, the invention features a pharmaceutical composition that includes a multi-targeted molecule (e.g., the effector molecules such as bis siRNA or sciRNA (or bis-sciRNA) compound), e.g., a double-stranded siRNA compound, or ssiRNA compound, (e.g., a precursor, e.g., a larger siRNA compound which can be processed into a ssiRNA compound, or a DNA which encodes an sciRNA (or bis-sciRNA) compound, e.g., a double-stranded siRNA compound, or ssiRNA compound, or precursor thereof) including a nucleotide sequence complementary to a target RNA, e.g., substantially and/or exactly complementary. The target RNA can be a transcript of an endogenous human gene. In one embodiment, the pharmaceutical composition can be an emulsion, microemulsion, cream, jelly, or liposome.

In one example, the pharmaceutical composition includes a multi-targeted molecule (e.g., the effector molecules such as bis siRNA or sciRNA (or bis-sciRNA) compound) mixed with a topical delivery agent. The topical delivery agent can be a plurality of microscopic vesicles. The microscopic vesicles can be liposomes. In some embodiments the liposomes are cationic liposomes.

In another aspect, the pharmaceutical composition includes a multi-targeted molecule (e.g., the effector molecules such as bis siRNA or sciRNA (or bis-sciRNA) compound) admixed with a topical penetration enhancer. In one embodiment, the topical penetration enhancer is a fatty acid. The fatty acid can be arachidonic acid, oleic acid, lauric acid, caprylic acid, capric acid, myristic acid, palmitic acid, stearic acid, linoleic acid, linolenic acid, dicaprate, tricaprate, monolein, dilaurin, glyceryl 1-monocaprate, 1-dodecylazacycloheptan-2-one, an acylcarnitine, an acylcholine, or a C₁₋₁₀ alkyl ester, monoglyceride, diglyceride or pharmaceutically acceptable salt thereof.

In another embodiment, the topical penetration enhancer is a bile salt. The bile salt can be cholic acid, dehydrocholic acid, deoxycholic acid, glucholic acid, glycholic acid, glycodeoxycholic acid, taurocholic acid, taurodeoxycholic acid, chenodeoxycholic acid, ursodeoxycholic acid, sodium tauro-24,25-dihydro-fusidate, sodium glycodihydrofusidate, polyoxyethylene-9-lauryl ether or a pharmaceutically acceptable salt thereof.

In another embodiment, the penetration enhancer is a chelating agent. The chelating agent can be EDTA, citric acid, a salicyclate, a N-acyl derivative of collagen, laureth-9, an N-amino acyl derivative of a beta-diketone or a mixture thereof.

In another embodiment, the penetration enhancer is a surfactant, e.g., an ionic or nonionic surfactant. The surfactant can be sodium lauryl sulfate, polyoxyethylene-9-lauryl ether, polyoxyethylene-20-cetyl ether, a perfluorchemical emulsion or mixture thereof.

In another embodiment, the penetration enhancer can be selected from a group consisting of unsaturated cyclic ureas, 1-alkyl-alkones, 1-alkenylazacyclo-alakanones, steroidal anti-inflammatory agents and mixtures thereof. In yet another embodiment the penetration enhancer can be a glycol, a pyrrol, an azone, or a terpene.

In one aspect, the invention features a pharmaceutical composition including a multi-targeted molecule (e.g., the effector molecules such as bis siRNA or sciRNA (or bis-sciRNA) compound) in a form suitable for oral delivery. In one embodiment, oral delivery can be used to deliver a multi-targeted molecule (e.g., the effector molecules such as bis siRNA or sciRNA (or bis-sciRNA) compound) composition to a cell or a region of the gastro-intestinal tract, e.g., small intestine, colon (e.g., to treat a colon cancer), and so forth. The oral delivery form can be tablets, capsules or gel capsules. In one embodiment, the the multi-targeted molecule (e.g., the effector molecules such as bis siRNA or sciRNA (or bis-sciRNA) compound) of the pharmaceutical composition modulates expression of a cellular adhesion protein, modulates a rate of cellular proliferation, or has biological activity against eukaryotic pathogens or retroviruses. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methylcellulose phthalate or cellulose acetate phthalate.

In another embodiment, the oral dosage form of the pharmaceutical composition includes a penetration enhancer. The penetration enhancer can be a bile salt or a fatty acid. The bile salt can be ursodeoxycholic acid, chenodeoxycholic acid, and salts thereof. The fatty acid can be capric acid, lauric acid, and salts thereof.

In another embodiment, the oral dosage form of the pharmaceutical composition includes an excipient. In one example, the excipient is polyethyleneglycol. In another example, the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

In one aspect, the invention features a pharmaceutical composition including an the multi-targeted molecule (e.g., the effector molecules such as bis siRNA or sciRNA (or bis-sciRNA) compound and a delivery vehicle.

In one embodiment, the delivery vehicle can deliver a multi-targeted molecule (e.g., the effector molecules such as bis siRNA or sciRNA (or bis-sciRNA) compound) to a cell by a topical route of administration. The delivery vehicle can be microscopic vesicles. In one example, the microscopic vesicles are liposomes. In some embodiments the liposomes are cationic liposomes. In another example, the microscopic vesicles are micelles.

Suitable topical formulations include those in which the multi-targeted molecules featured in the present disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Multi-targeted molecules featured in the present disclosure may be encapsulated within liposomes or may form complexes thereto, in particular to cationic liposomes. Alternatively, the multi-targeted molecules may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present disclosure, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

In one aspect, the invention features a pharmaceutical composition including a multi-targeted molecule (e.g., the effector molecules such as bis siRNA or sciRNA (or bis-sciRNA) compound) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including a multi-targeted molecule (e.g., the effector molecules such as bis siRNA or sciRNA (or bis-sciRNA) compound) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In one aspect, the invention features a pharmaceutical composition including a multi-targeted molecule (e.g., the effector molecules such as bis siRNA or sciRNA (or bis-sciRNA) compound) in a rectal dosage form. In one embodiment, the rectal dosage form is an enema. In another embodiment, the rectal dosage form is a suppository.

In one aspect, the invention features a pharmaceutical composition including a multi-targeted molecule (e.g., the effector molecules such as bis siRNA or sciRNA (or bis-sciRNA) compound) in a vaginal dosage form. In one embodiment, the vaginal dosage form is a suppository. In another embodiment, the vaginal dosage form is a foam, cream, or gel.

In one aspect, the invention features a pharmaceutical composition including a multi-targeted molecule (e.g., the effector molecules such as bis siRNA or sciRNA (or bis-sciRNA) compound) in a pulmonary or nasal dosage form. In one embodiment, the sciRNA (or bis-sciRNA) compound is incorporated into a particle, e.g., a macroparticle, e.g., a microsphere. The particle can be produced by spray drying, lyophilization, evaporation, fluid bed drying, vacuum drying, or a combination thereof. The microsphere can be formulated as a suspension, a powder, or an implantable solid.

### Treatment Methods and Routes of Delivery

Another aspect of the invention relates to a method of reducing the expression of a target gene in a cell, comprising contacting said cell with the multi-targeted molecule (e.g., the effector molecules such as bis siRNA or sciRNA (or bis-sciRNA) agent). In one embodiment, the cell is a heptic cell. In one embodiment, the cell is an extraheptic cell.

Another aspect of the invention relates to a method of reducing the expression of a target gene in a subject, comprising administering to the subject the multi-targeted molecule (e.g., the effector molecules such as bis siRNA or sciRNA (or bis-sciRNA) agent).

The multi-targeted molecule (e.g., the effector molecules such as bis siRNA or sciRNA (or bis-sciRNA) agent) can be delivered to a subject by a variety of routes, depending on the type of genes targeted and the type of disorders to be treated. In some embodiments, the multi-targeted molecule (e.g., the effector molecules such as bis siRNA or sciRNA (or bis-sciRNA) agent) is administered hepatically. In some embodiments, the multi-targeted molecule (e.g., the effector molecules such as bis siRNA or sciRNA (or bis-sciRNA) agent) is administered extrahepatically, such as an ocular administration (e.g., intravitreal administration) or an intrathecal administration.

In one embodiment, the multi-targeted molecule (e.g., the effector molecules such as bis siRNA or sciRNA (or bis-sciRNA) agent) is administered intrathecally. By intrathecal administration of the multi-targeted molecule (e.g., the effector molecules such as bis siRNA or sciRNA (or bis-sciRNA) agent), the method can reduce the expression of a target gene in a brain or spine tissue, for instance, cortex, cerebellum, cervical spine, lumbar spine, and thoracic spine.

For ease of exposition the formulations, compositions and methods in this section are discussed largely with regard to modified effector molecule or sciRNA compounds. It may be understood, however, that these formulations, compositions and methods can be practiced with other effector molecule or siRNA compounds, e.g., unmodified effector molecule or sciRNA compounds, and such practice is within the invention. A composition that includes a iRNA can be delivered to a subject by a variety of routes. Exemplary routes include: intravenous, topical, rectal, anal, vaginal, nasal, pulmonary, ocular.

The multi-targeted molecule (e.g., the effector molecules such as bis siRNA or sciRNA (or bis-sciRNA) molecules) can be incorporated into pharmaceutical compositions suitable for administration. Such compositions typically include one or more species of multi-targeted molecules (e.g., the effector molecules such as bis siRNA or sciRNA (or bis-sciRNA)) and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the iRNA in aerosol form. The vascular endothelial cells could be targeted by coating a balloon catheter with the iRNA and mechanically introducing the DNA.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes may be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

In one embodiment, the administration of the multi-targeted molecule (e.g., the effector molecules such as bis siRNA or sciRNA (or bis-sciRNA) compound) composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, the multi-targeted molecule (e.g., the effector molecules such as bis siRNA or sciRNA (or bis-sciRNA) agent) is delivered by intrathecal injection (i.e. injection into the spinal fluid which bathes the brain and spinal chord tissue). Intrathecal injection of iRNA agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal chord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in PCT/US2015/013253, filed on Jan. 28, 2015, which is incorporated by reference in its entirety.

The amount of intrathecally injected iRNA agents may vary from one target gene to another target gene and the appropriate amount that has to be applied may have to be determined individually for each target gene. Typically, this amount ranges between 10 μg to 2 mg, preferably 50 μg to 1500 μg, more preferably 100 μg to 1000 μg.

Rectal Administration. The invention also provides methods, compositions, and kits, for rectal administration or delivery of multi-targeted molecule (e.g., the effector molecules such as bis siRNA or sciRNA (or bis-sciRNA) compounds) described herein.

Accordingly, an multi-targeted molecule (e.g., the effector molecules such as bis siRNA or sciRNA (or bis-sciRNA) compound) can be administered rectally, e.g., introduced through the rectum into the lower or upper colon. This approach is particularly useful in the treatment of, inflammatory disorders, disorders characterized by unwanted cell proliferation, e.g., polyps, or colon cancer.

The medication can be delivered to a site in the colon by introducing a dispensing device, e.g., a flexible, camera-guided device similar to that used for inspection of the colon or removal of polyps, which includes means for delivery of the medication.

The rectal administration of the multi-targeted molecule (e.g., the effector molecules such as bis siRNA or sciRNA (or bis-sciRNA) compound) is by means of an enema. The multi-targeted molecule (e.g., the effector molecules such as bis siRNA or sciRNA (or bis-sciRNA) compound) of the enema can be dissolved in a saline or buffered solution. The rectal administration can also by means of a suppository, which can include other ingredients, e.g., an excipient, e.g., cocoa butter or hydropropylmethylcellulose.

Ocular Delivery. The multi-targeted molecule (e.g., the effector molecules such as bis siRNA or sciRNA (or bis-sciRNA) agents) described herein can be administered to an ocular tissue. For example, the medications can be applied to the surface of the eye or nearby tissue, e.g., the inside of the eyelid. They can be applied topically, e.g., by spraying, in drops, as an eyewash, or an ointment. Administration can be provided by the subject or by another person, e.g., a health care provider. The medication can be provided in measured doses or in a dispenser which delivers a metered dose. The medication can also be administered to the interior of the eye, and can be introduced by a needle or other delivery device which can introduce it to a selected area or structure. Ocular treatment is particularly desirable for treating inflammation of the eye or nearby tissue.

In certain embodiments, the multi-targeted molecule (e.g., the effector molecules such as bis siRNA or sciRNA (or bis-sciRNA) agents) may be delivered directly to the eye by ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

In one embodiment, the multi-targeted molecule (e.g., the effector molecules such as bis siRNA or sciRNA (or bis-sciRNA) agents may be administered into the eye, for example the vitreous chamber of the eye, by intravitreal injection, such as with pre-filled syringes in ready-to-inject form for use by medical personnel.

For ophthalmic delivery, the multi-targeted molecule (e.g., the effector molecules such as bis siRNA or sciRNA (or bis-sciRNA) agents may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the conjugate in a physiologically acceptable isotonic aqueous buffer. Further, the solution may include an acceptable surfactant to assist in dissolving the multi-targeted molecule (e.g., the effector molecules such as bis siRNA or sciRNA (or bis-sciRNA) agents). Viscosity building agents, such as hydroxymethyl cellulose, hydroxyethyl cellulose, methylcellulose, polyvinylpyrrolidone, or the like may be added to the pharmaceutical compositions to improve the retention of the multi-targeted molecule (e.g., the effector molecules such as bis siRNA or sciRNA (or bis-sciRNA) agents).

To prepare a sterile ophthalmic ointment formulation, the multi-targeted molecule (e.g., the effector molecules such as bis siRNA or sciRNA (or bis-sciRNA) agents) is combined with a preservative in an appropriate vehicle, such as mineral oil, liquid lanolin, or white petrolatum. Sterile ophthalmic gel formulations may be prepared by suspending the multi-targeted molecule (e.g., the effector molecules such as bis siRNA or sciRNA (or bis-sciRNA) agents) in a hydrophilic base prepared from the combination of, for example, CARBOPOL®-940 (BF Goodrich, Charlotte, N.C.), or the like, according to methods known in the art.

Topical Delivery. Any of the multi-targeted molecule (e.g., the effector molecules such as bis siRNA or sciRNA (or bis-sciRNA) compounds) described herein can be administered directly to the skin. For example, the medication can be applied topically or delivered in a layer of the skin, e.g., by the use of a microneedle or a battery of microneedles which penetrate into the skin, but, for example, not into the underlying muscle tissue. Administration of the multi-targeted molecule (e.g., the effector molecules such as bis siRNA or sciRNA (or bis-sciRNA) compound) composition can be topical. Topical applications can, for example, deliver the composition to the dermis or epidermis of a subject. Topical administration can be in the form of transdermal patches, ointments, lotions, creams, gels, drops, suppositories, sprays, liquids or powders. A composition for topical administration can be formulated as a liposome, micelle, emulsion, or other lipophilic molecular assembly. The transdermal administration can be applied with at least one penetration enhancer, such as iontophoresis, phonophoresis, and sonophoresis.

In some embodiments, a multi-targeted molecule (e.g., the effector molecules such as bis siRNA or sciRNA (or bis-sciRNA) compound) is delivered to a subject via topical administration. “Topical administration” refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

The term “skin,” as used herein, refers to the epidermis and/or dermis of an animal. Mammalian skin consists of two major, distinct layers. The outer layer of the skin is called the epidermis. The epidermis is comprised of the stratum corneum, the stratum granulosum, the stratum spinosum, and the stratum basale, with the stratum corneum being at the surface of the skin and the stratum basale being the deepest portion of the epidermis. The epidermis is between 50 μm and 0.2 mm thick, depending on its location on the body.

Beneath the epidermis is the dermis, which is significantly thicker than the epidermis. The dermis is primarily composed of collagen in the form of fibrous bundles. The collagenous bundles provide support for, inter alia, blood vessels, lymph capillaries, glands, nerve endings and immunologically active cells.

One of the major functions of the skin as an organ is to regulate the entry of substances into the body. The principal permeability barrier of the skin is provided by the stratum corneum, which is formed from many layers of cells in various states of differentiation. The spaces between cells in the stratum corneum is filled with different lipids arranged in lattice-like formations that provide seals to further enhance the skins permeability barrier.

The permeability barrier provided by the skin is such that it is largely impermeable to molecules having molecular weight greater than about 750 Da. For larger molecules to cross the skin's permeability barrier, mechanisms other than normal osmosis must be used.

Several factors determine the permeability of the skin to administered agents. These factors include the characteristics of the treated skin, the characteristics of the delivery agent, interactions between both the drug and delivery agent and the drug and skin, the dosage of the drug applied, the form of treatment, and the post treatment regimen. To selectively target the epidermis and dermis, it is sometimes possible to formulate a composition that comprises one or more penetration enhancers that will enable penetration of the drug to a preselected stratum.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy.

In addition, iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

The compositions and methods provided may also be used to examine the function of various proteins and genes in vitro in cultured or preserved dermal tissues and in animals. The invention can be thus applied to examine the function of any gene. The methods of the invention can also be used therapeutically or prophylactically. For example, for the treatment of animals that are known or suspected to suffer from diseases such as psoriasis, lichen planus, toxic epidermal necrolysis, ertythema multiforme, basal cell carcinoma, squamous cell carcinoma, malignant melanoma, Paget's disease, Kaposi's sarcoma, pulmonary fibrosis, Lyme disease and viral, fungal and bacterial infections of the skin.

Pulmonary Delivery. Any of the multi-targeted molecule (e.g., the effector molecules such as bis siRNA or sciRNA (or bis-sciRNA) compounds) described herein can be administered to the pulmonary system. Pulmonary administration can be achieved by inhalation or by the introduction of a delivery device into the pulmonary system, e.g., by introducing a delivery device which can dispense the medication. Certain embodiments may use a method of pulmonary delivery by inhalation. The medication can be provided in a dispenser which delivers the medication, e.g., wet or dry, in a form sufficiently small such that it can be inhaled. The device can deliver a metered dose of medication. The subject, or another person, can administer the medication. Pulmonary delivery is effective not only for disorders which directly affect pulmonary tissue, but also for disorders which affect other tissue. Multi-targeted molecule (e.g., the effector molecules such as bis siRNA or sciRNA (or bis-sciRNA) compounds) can be formulated as a liquid or nonliquid, e.g., a powder, crystal, or aerosol for pulmonary delivery.

A composition that includes an multi-targeted molecule (e.g., the effector molecules such as bis siRNA or sciRNA (or bis-sciRNA) compound) can be administered to a subject by pulmonary delivery. Pulmonary delivery compositions can be delivered by inhalation by the patient of a dispersion so that the composition, for example, iRNA, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are may be used. One of the benefits of using an atomizer or inhaler is that the potential for contamination is minimized because the devices are self contained. Dry powder dispersion devices, for example, deliver drugs that may be readily formulated as dry powders. A iRNA composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” For example, the average particle size is less than about 10 μm in diameter with a relatively uniform spheroidal shape distribution. In some embodiments, the diameter is less than about 7.5 μm and in some embodiments less than about 5.0 μm. Usually the particle size distribution is between about 0.1 μm and about 5 μm in diameter, sometimes about 0.3 μm to about 5 μm.

The term “dry” means that the composition has a moisture content below about 10% by weight (% w) water, usually below about 5% w and in some cases less it than about 3% w. A dry composition can be such that the particles are readily dispersible in an inhalation device to form an aerosol.

The term “therapeutically effective amount” is the amount present in the composition that is needed to provide the desired level of drug in the subject to be treated to give the anticipated physiological response.

The term “physiologically effective amount” is that amount delivered to a subject to give the desired palliative or curative effect.

The term “pharmaceutically acceptable carrier” means that the carrier can be taken into the lungs with no significant adverse toxicological effects on the lungs.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. A group of carbohydrates may include lactose, threhalose, raffinose maltodextrins, and mannitol. Suitable polypeptides include aspartame. Amino acids include alanine and glycine, with glycine being used in some embodiments.

Additives, which are minor components of the composition of this invention, may be included for conformational stability during spray drying and for improving dispersibility of the powder. These additives include hydrophobic amino acids such as tryptophan, tyrosine, leucine, phenylalanine, and the like.

Suitable pH adjusters or buffers include organic salts prepared from organic acids and bases, such as sodium citrate, sodium ascorbate, and the like; sodium citrate may be used in some embodiments.

Pulmonary administration of a micellar iRNA formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Oral or Nasal Delivery. Any of the multi-targeted molecule (e.g., the effector molecules such as bis siRNA or sciRNA (or bis-sciRNA) compounds) described herein can be administered orally, e.g., in the form of tablets, capsules, gel capsules, lozenges, troches or liquid syrups. Further, the composition can be applied topically to a surface of the oral cavity.

Any of the multi-targeted molecule (e.g., the effector molecules such as bis siRNA or sciRNA (or bis-sciRNA) compounds) described herein can be administered nasally. Nasal administration can be achieved by introduction of a delivery device into the nose, e.g., by introducing a delivery device which can dispense the medication. Methods of nasal delivery include spray, aerosol, liquid, e.g., by drops, or by topical administration to a surface of the nasal cavity. The medication can be provided in a dispenser with delivery of the medication, e.g., wet or dry, in a form sufficiently small such that it can be inhaled. The device can deliver a metered dose of medication. The subject, or another person, can administer the medication.

Nasal delivery is effective not only for disorders which directly affect nasal tissue, but also for disorders which affect other tissue the multi-targeted molecule (e.g., the effector molecules such as bis siRNA or sciRNA (or bis-sciRNA) compounds) can be formulated as a liquid or nonliquid, e.g., a powder, crystal, or for nasal delivery. As used herein, the term “crystalline” describes a solid having the structure or characteristics of a crystal, i.e., particles of three-dimensional structure in which the plane faces intersect at definite angles and in which there is a regular internal structure. The compositions of the invention may have different crystalline forms. Crystalline forms can be prepared by a variety of methods, including, for example, spray drying.

Both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many drugs. Further, the sublingual mucosa is convenient, acceptable and easily accessible.

The ability of molecules to permeate through the oral mucosa appears to be related to molecular size, lipid solubility and peptide protein ionization. Small molecules, less than 1000 daltons appear to cross mucosa rapidly. As molecular size increases, the permeability decreases rapidly. Lipid soluble compounds are more permeable than non-lipid soluble molecules. Maximum absorption occurs when molecules are un-ionized or neutral in electrical charges. Therefore charged molecules present the biggest challenges to absorption through the oral mucosae.

A pharmaceutical composition of the multi-targeted molecule (e.g., the effector molecules such as bis siRNA or sciRNA (or bis-sciRNA)) may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity. For example, the medication can be sprayed into the buccal cavity or applied directly, e.g., in a liquid, solid, or gel form to a surface in the buccal cavity. This administration is particularly desirable for the treatment of inflammations of the buccal cavity, e.g., the gums or tongue, e.g., in one embodiment, the buccal administration is by spraying into the cavity, e.g., without inhalation, from a dispenser, e.g., a metered dose spray dispenser that dispenses the pharmaceutical composition and a propellant.

### Kits

In certain other aspects, the invention provides kits that include a suitable container containing a pharmaceutical formulation of an effector molecule (e.g., an dsRNA agent) or an sciRNA (or bis-sciRNA) compound. In certain embodiments the individual components of the pharmaceutical formulation may be provided in one container. Alternatively, it may be desirable to provide the components of the pharmaceutical formulation separately in two or more containers, e.g., one container for an effector molecule (e.g., an dsRNA agent) or an sciRNA (or bis-sciRNA) compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The invention is further illustrated by the following examples, which should not be construed as further limiting. The contents of all references, pending patent applications and published patents, cited throughout this application are hereby expressly incorporated by reference.

### Research Tools

In certain instances, oligonucleotides capable of modulating gene expression have been used as research tools. For example, researchers investigating the function of a particular gene product can design oligonucleotides to reduce the amount of that gene product present in a cell or an animal and observe phenotypic changes in the cell or animal. In certain embodiments, the present disclosure provides methods for reducing the amount of two different targets in a cell or animal. In some embodiments, the two different targets can be two different genes or gene products. In some embodiments, the two different targets can be the same gene or gene product. In certain embodiments, investigators can use such techniques to characterize proteins or untranslated nucleic acids. In certain embodiments, such experiments are used to investigate kinetics and/or turnover of gene products and/or certain cellular functions. In some embodiments, such experiments are used to investigate relationship or correlation between different genes or gene products.

### EXAMPLES

The invention now being generally described, it will be more readily understood by reference to the following examples which are included merely for purposes of illustration of certain aspects and embodiments of the present invention, and are not intended to limit the invention.

### Example 1: Methods for Preparing Bis siRNA Complexes

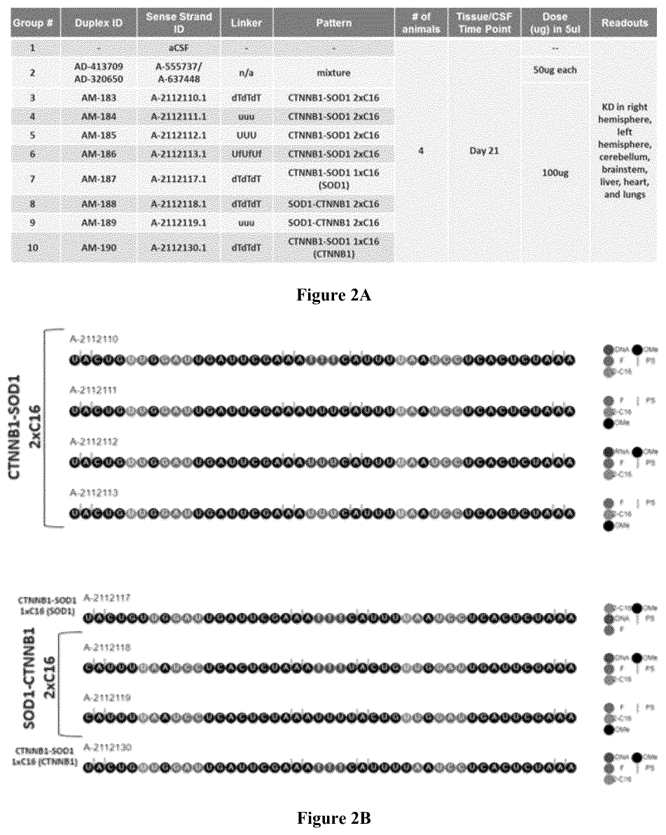

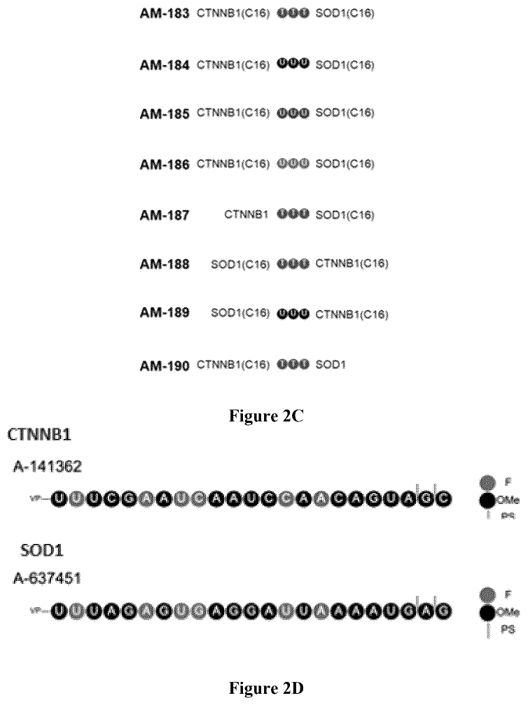

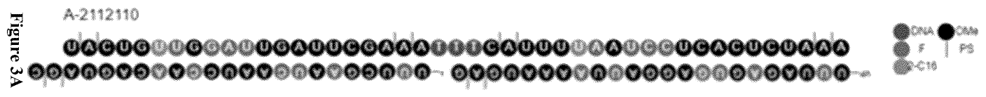

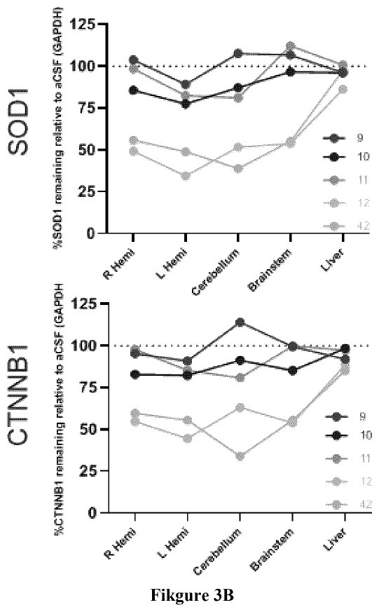

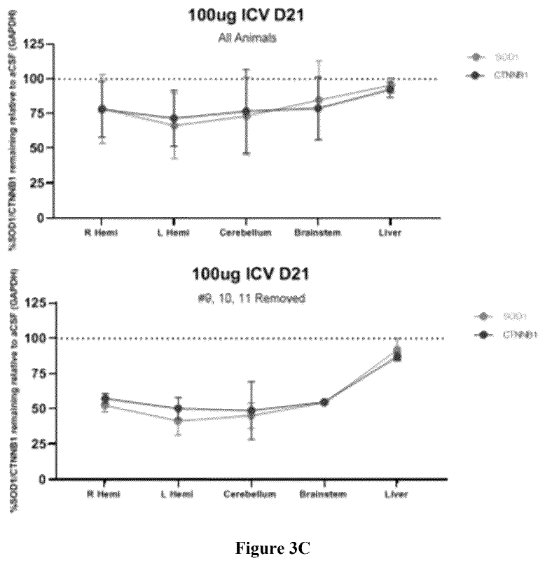

Multiple “bis” (dual-targeting) siRNA complexes (a form of multiple-targeted molecule as disclosed herein) were prepared, using synthesis methods previously described in PCT application no. PCT/US2016/042498. The “bis” siRNA complexes were designed for simultaneous (and stoichiometrically equivalent with respect to effector molecule concentration) inhibition of CTNNB1 and SOD1 nucleic acid targets (FIGS. 2A-2C). Each bis siRNA complex included a continuous sense strand that respectively included a sense strand sequence within a first target nucleic acid (CTNNB1 or SOD1 mRNA) and a sense strand sequence within a second target nucleic acid (the other of CTNNB1 or SOD1 mRNA), the respective sense strands of the individual effector molecules (siRNAs) joined by a nucleotide linker of three nucleotides in length (as presently exemplified), and two separate and independent single-stranded antisense sequences that respectively target the first target nucleic acid and the second target nucleic acid and respectively anneal to complementary sense strand sequences of the continuous sense strand of the bis siRNA complex (FIG. 2D), with an exemplary bis siRNA complex (multi-targeted molecule) shown in FIG. 3A.

The efficacy and tissue distribution of each bis siRNA (or mixed duplex control) was evaluated in the central nervous system of C57/BI6 mice. Single ICV administrations to the right ventricle of the mouse brain were performed using a 3.5 mm custom needle with a 25 ml Hamilton syringe, using isoflurane as anesthesia. 50 mg of each duplex was administered in the mixture duplex control. 100 mg of the bis siRNA complexes were administered. All injections were performed by the same administrator. At least 4 animals were used per test compound. Animals were euthanized on day 21 post-administration. Inhibition of CTNNB1 and SOD1 was evaluated in the right and left hemispheres of the brain, cerebellum, brain stem, liver, heart, and lungs.

### Example 2: Identification of Effective Bis siRNA Complex Linkers for CNS Delivery

Bis siRNA complexes of the instant disclosure were constructed using respective C₁₆-modified SOD1- and CTNNB1-targeting siRNA molecules, as tabulated in FIG. 2A. When administered to mice via ICV injection as a simple mixture of siRNAs (that were not linked), the indicated C₁₆-modified SOD1- and CTNNB1-targeting siRNA molecules were respectively identified to inhibit SOD1 by about 40%-60% in various brain tissues and CTNNB1 by about 25%-50% (FIG. 2B).

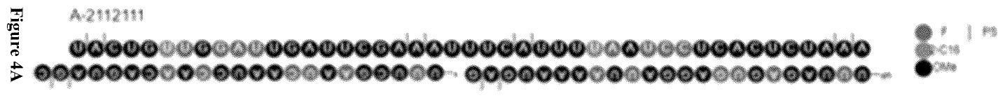

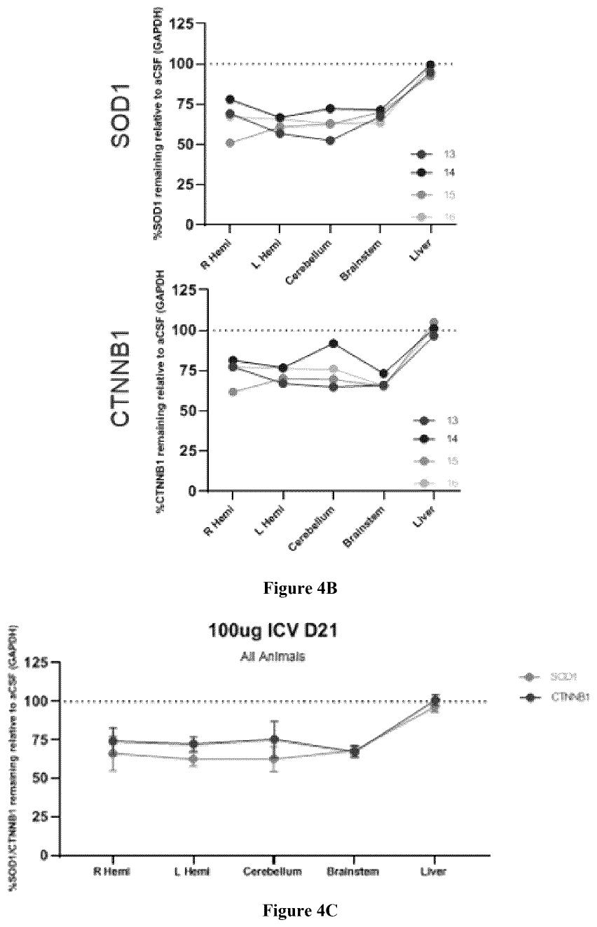

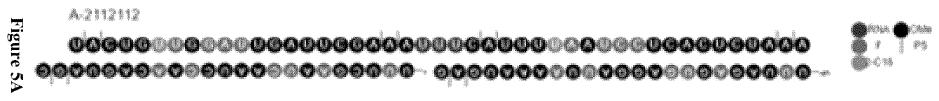

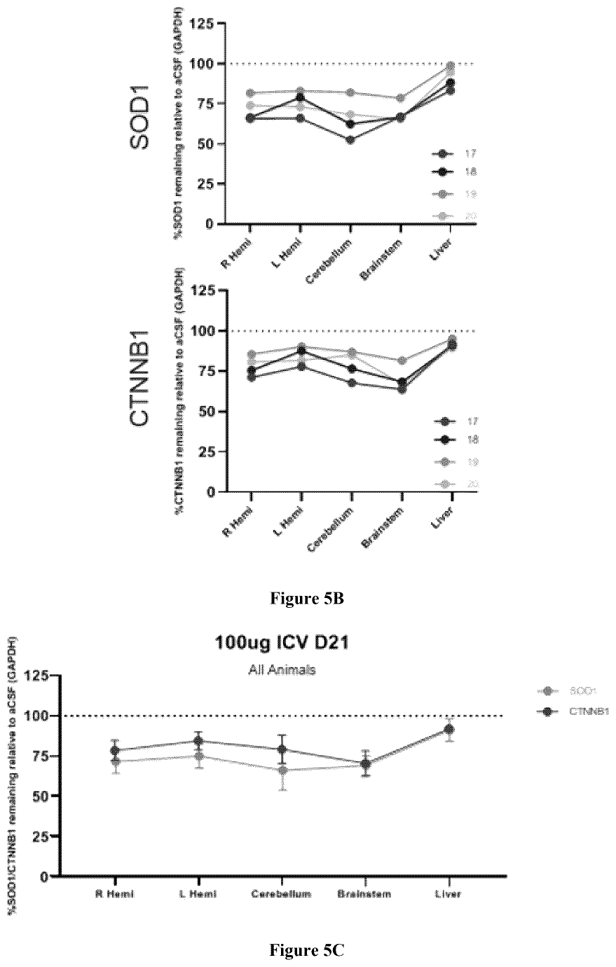

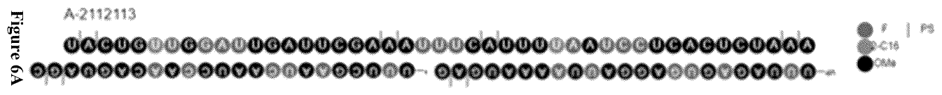

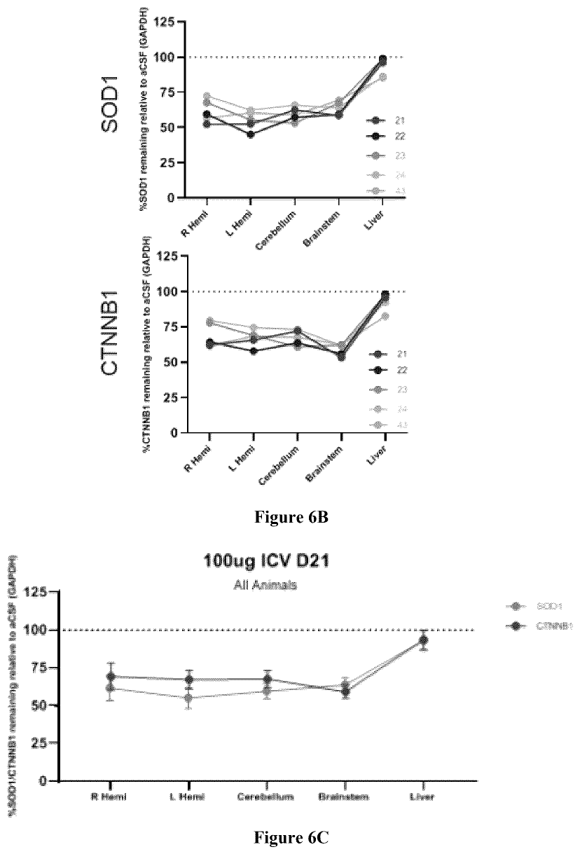

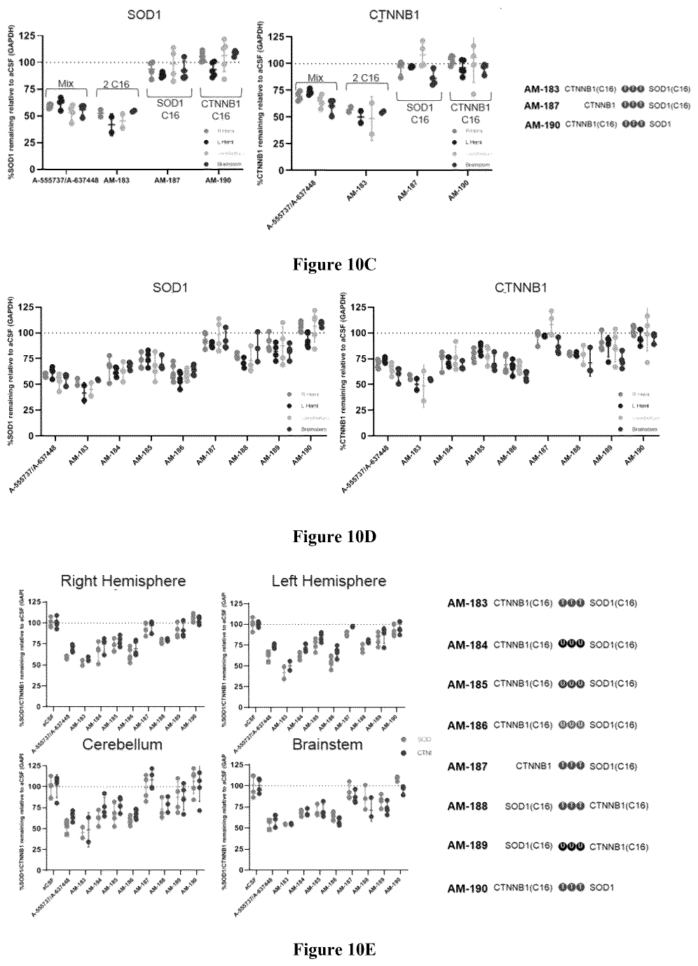

To identify effective linkers for the bis siRNA complexes disclosed herein, bis siRNA complex-mediated inhibition of targeted mSOD1 and mCTNNB1 was assessed in mice, with target mRNA knockdown levels also compared to mixed duplex delivery (siRNAs of SEQ ID NOs: 13 and 14, targeting SOD1, and SEQ ID NOs: 15 and 16, targeting CTTNB1, respectively). Mice were injected with bis siRNA complexes possessing one of three sense-strand-joining nucleotide linkers: 2′-deoxythymidine-3′-phosphate (dTdTdT) SEQ ID NO: 3 (FIGS. 3A-3C), 2′O-methyl uridine (uuu) SEQ ID NO: 4 (FIGS. 4A-4C), uridine-3′-phosphate (UUU) SEQ ID NO: 5 (FIGS. 5A-5C), and 2′-fluorouridine-3′-phosphate (UfUfUf) SEQ ID NO: 6 (FIGS. 6A-6C). Tissues collected for knock down assessment were the right hemisphere of the brain, left hemisphere of the brain, cerebellum, brainstem, and liver. About 50% inhibition of both mSOD1 and mCTNNB1 levels was achieved with the 2′-deoxythymidine-3′-phosphate (dTdTdT) linker construct CTNNB1(C16)-SOD1(C16) bis siRNA (FIGS. 3A-3C). About 30% inhibition of mSOD1 and 25% inhibition of mCTNNB1 levels was achieved with a 2′O-methyl uridine (uuu) linker construct CTNNB1(C16)-SOD1(C16) bis siRNA (FIGS. 4A-4C). Only about 25% inhibition of both mSOD1 and mCTNNB1 levels was achieved with a uridine-3′-phosphate (UUU) linker construct CTNNB1(C16)-SOD1(C16) bis siRNA (FIGS. 5A-5C). About 40% inhibition of mSOD1 and 30% inhibition of mCTNNB1 was achieved with a 2′-fluorouridine-3′-phosphate (UfUfUf) linker construct CTNNB1(C16)-SOD1(C16) bis siRNA (FIGS. 6A-6C). Compared to both mixed duplex and other bis siRNA linkers, the most effective bis siRNA linker construct observed for inhibition of both SOD1 and CTNNB1 in the CNS was identified as the dTdTdT-linked CTNNB1(C16)-SOD1(C16) bis siRNA (FIG. 10A), which exhibited about 50% inhibition of both SOD1 and CTNNB1 across all brain tissues examined.

### Example 3: Impact of Bis siRNA Effector Molecule Placement in Multi-Targeted Molecules

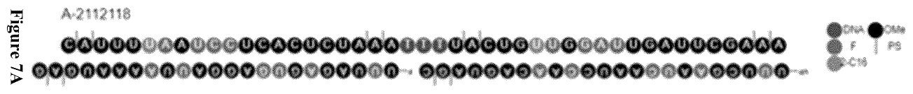

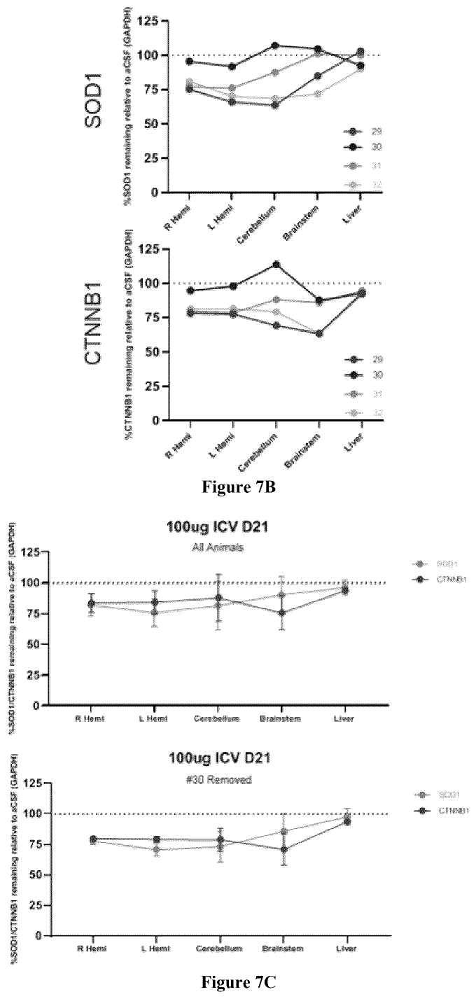

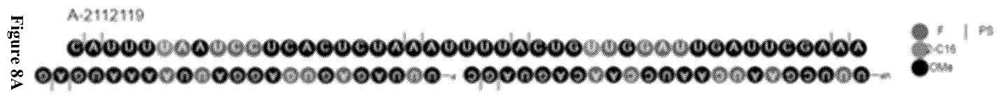

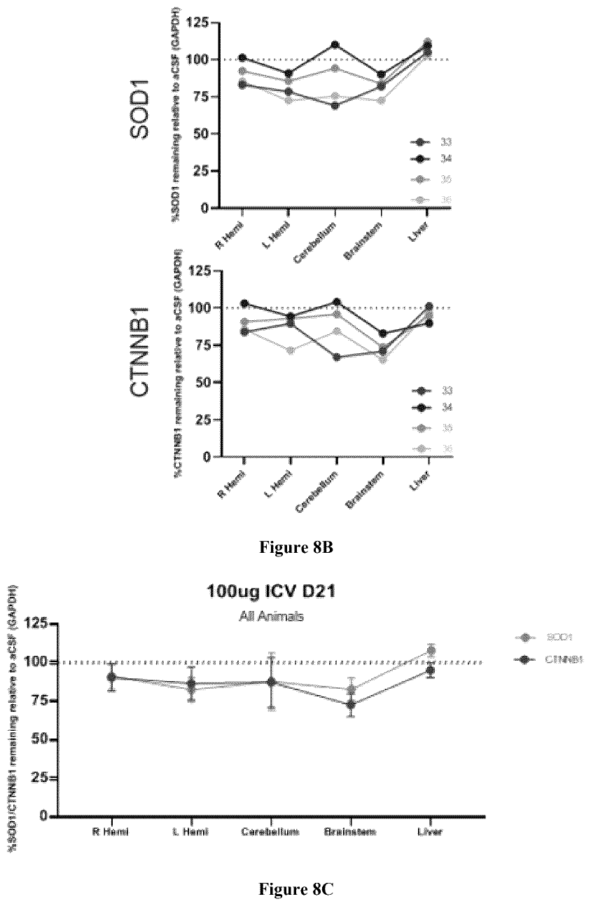

The potential impact of effector molecule configuration upon multi-targeted molecule activity was assessed using respective SOD1-targeting and CTNNB1-targeting siRNA effector molecules described above, yet swapping the position of the respective siRNA effectors within the bis siRNA complexes (multi-targeted molecules). Inhibition of mSOD1 and mCTNNB1 was assessed in mice injected ICV with bis siRNAs comprising SOD1(C16)-CTNNB1(C16) fused sense strand sequences (SEQ ID NOs: 22 and 23). As above, tissues collected for assessment of target gene inhibition were the right hemisphere of the brain, left hemisphere of the brain, cerebellum, brainstem, and liver. In contrast to the robust mSOD1 and mCTNNB1 inhibitory activity observed for the above-tested CTNNB1(C16)-SOD1(C16) bis siRNA complex having a a DNA linker (dTdTdT, see FIGS. 3A-3C), a SOD1(C16)-CTNNB1(C16) bis siRNA complex also having a DNA linker (dTdTdT)—and therefore only differing from the CTNNB1(C16)-SOD1(C16) bis siRNA complex in the ordering of siRNA effector molecules within the bis siRNA construct—exhibited only about 20% inhibition of both mSOD1 and mCTNNB1 target mRNAs (FIGS. 7A-7C). A similarly diminished effect was observed for a SOD1(C16)-CTNNB1(C16) bis siRNA complex having a 2′O-methyl linker (uuu)—which therefore only differed from the CTNNB1(C16)-SOD1(C16) bis siRNA complex having a 2′O-methyl linker (FIGS. 4A-4C) in the ordering of siRNA effector molecules within the bis siRNA construct—with the SOD1(C16)-CTNNB1(C16) bis siRNA complex having a 2′O-methyl linker (uuu) observed only to inhibit mSOD1 and mCTNNB1 target mRNAs by about 15% (FIGS. 8A-8C), as contrasted with the about 25% to 30% inhibition observed above for the CTNNB1(C16)-SOD1(C16) bis siRNA complex having a 2′O-methyl linker (FIGS. 4A-4C). Even as compared to a mixed duplex treatment and all other bis siRNA complexes tested, the most effective bis siRNA configuration was observed to be CTNNB1(C16)-SOD1(C16), at about 50% knock down of both SOD1 and CTNNB1 (FIG. 10B).

### Example 4: Multi-Targeted Molecules Having Only a Single C₁₆ Modification Per Multi-Targeted Molecule, Positioned on Either siRNA Effector Molecule, Rather than on Each siRNA Effector Molecule, Performed Poorly

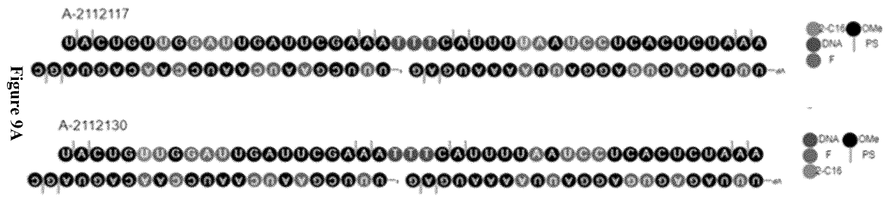

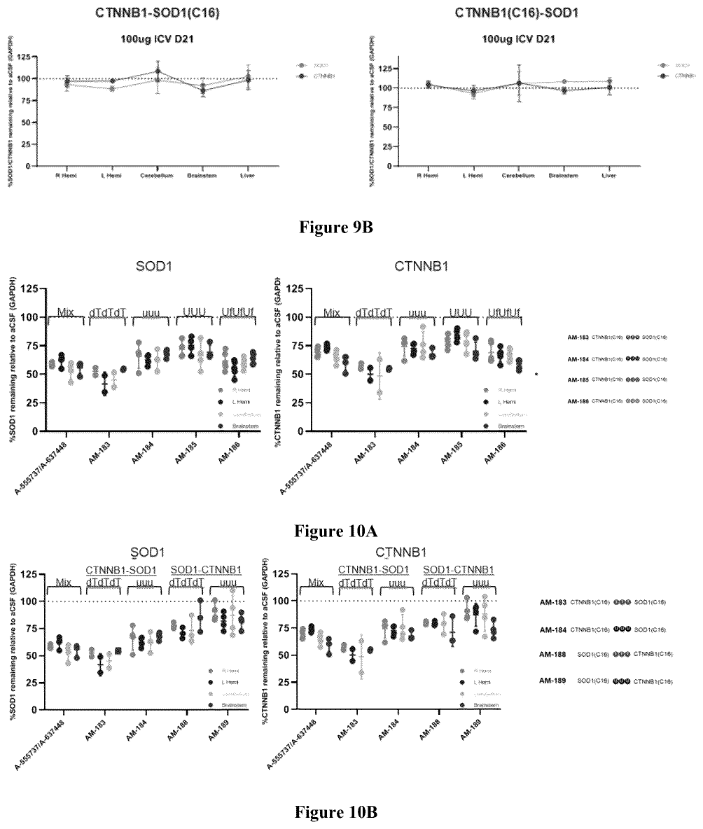

Inhibitory efficacy of bis siRNA complexes harboring only a single C₁₆ modification at sense strand position 6 (numbering from the 5′-terminal end) of either the SOD1-targeting siRNA effector molecule or the CTNNB1-targeting effector molecule were also assessed for inhibitory efficacy. Remarkably, when either (1) a CTNNB1-SOD1(C16) bis siRNA complex joined by a DNA linker (the bis siRNA complex possessing a single C₁₆ modification at SOD1-targeting siRNA sense strand position 6; FIG. 9A at top, SEQ ID NO: 21 fused sense strand) or (2) a CTNNB1(C16)-SOD1 bis siRNA complex joined by a DNA linker (the bis siRNA complex having a single C₁₆ modification at CTNNB1-targeting siRNA sense strand position 6; FIG. 9A at bottom, SEQ ID NO: 24 fused sense strand) were ICV injected into mice and tested for CTNNB1 and SOD1 inhibition, neither bis siRNA complex exhibited inhibitory efficacy in any of the brain tissues examined (right hemisphere of the brain, left hemisphere of the brain, cerebellum, brainstem), nor in liver (FIG. 9B). These results were in stark contrast to the inhibitory efficacy observed in vivo in CNS tissues for bis siRNA complexes harboring a C₁₆ modification at sense strand position 6 of both effector molecules (both CTNNB1 and SOD1 siRNAs; see FIGS. 3A-3C and FIG. 10C).

In summary, a number of bis siRNA complexes were synthesized and tested for CTNNB1 and SOD1 target gene inhibitory efficacy in the CNS of mammalian subjects administered such multi-targeting molecules via ICV injection. Robustly effective and specific inhibition of both CTNNB1 and SOD1 target genes was observed for a number of CTNNB1(C16)-SOD1(C16) siRNA constructs, particularly those having a DNA (dTdTdT) linker (FIGS. 10D and 10E) and two distinct lipophilic moieties (C₁₆ modifications), with each siRNA effector molecule conjugated to a C₁₆ modification at position 6 of the sense strand. Inclusion of a conjugated lipophilic moiety (a C₁₆ modification as exemplified) upon both siRNA effector molecules was identified as important for assuring the target gene knockdown efficacy of such multi-targeted molecules in CNS tissues of a treated subject, with bis siRNA constructs possessing only a single C₁₆ modification upon one siRNA effector molecule but not both observed to be ineffective inhibitors of targeted genes, even of the gene targeted by the effector molecule harboring a single C₁₆ modification (FIGS. 10D and 10E). The composition of linkers and the configuration of individual siRNA effector molecules with respect to one another within a bis siRNA construct were further identified to impact target gene inhibitory efficacy of the bis siRNA constructs in CNS tissues of treated subjects (FIGS. 10D and 10E).

Accordingly, multi-targeted molecules capable of robust and effective CNS delivery and target gene inhibition have been identified and are provided herein.

### Example 5: Qualitative Stability of Bis-siRNA in Rat Cerebral Spinal Fluid (CSF) Using Polyacrylamide Gel Electrophoresis

Stability of bis-siRNA multiplex linkers were evaluated in rat CSF (BioIVT, Cat. RAT00CSFXZN) via non-denaturing polyacrylamide gel electrophoresis (PAGE) using Criterion 10% TBE precast gels (BioRad, Cat. 3450053). Bis-siRNA, mixture of duplexes (AD-320650 and AD-413709), and control DNA duplex (AD-77748) were incubated at 0.1 μM in CSF at 1:19 ratio for 0, 4, or 24 h, after which the reaction was quenched using EDTA (50 mM) and froze at −80° C. until analysis. Control samples of multiplexes incubated in PBS with no matrix added were also prepared. Prior to loading, samples were thawed at RT and BlueJuice™ Gel Loading Buffer (Life Technologies, Cat. 10816-015) was added in 1:10 ratio. Samples and 10 bp DNA Ladder (Life Technologies, Cat. 10488-019) were then loaded onto a 10% TBE gel in 1×TBE (Tris/Boric Acid/EDTA; BioRad, Cat. 171-0070) running buffer and constant voltage (100V) was applied for 2 h at 4° C. Gels were stained with Syber Gold (1× in 10% TBE; Life technologies, Cat. S11494) for 10 min and imaged using ChemiDoc Imaging System equipped with Image Lab software (BioRad).

### Semi-Quantitative Stability of Bis(siRNA) in Rat Brain Homogenate

Stability of bis-siRNA multiplex linkers were evaluated in a custom premium formulation prepared from female Sprague-Dawley rat whole brain tissue (BioIVT, Cat. S05966) formulated as 1 g tissue per 3 mL buffer (50 mM Tris HCl, 1% NP-40, 140 mM NaCl with cOmplete EDTA-free Protease Inhibitor Cocktail (Sigma Aldrich, Cat. 11836170001), pH to 7.4), and the metabolized were analyzed via high-accuracy high-resolution mass spectrometry. Bis-siRNAs (˜8 μM) were incubated with brain at 1:50 ratio for 0 or 24 h. The mixture was diluted with 450 μL lysis buffer (Phenomenex, Cat. AL0-8579) and solid phase extraction (SPE) was performed using Clarity OTX solid phase extraction plates (Phenomenex, Cat. 8E-S103-EGA) with positive pressure facilitated by ExtraHera (Biotage) instrument. Columns were conditioned with 1 mL methanol, followed by 1.9 mL equilibration buffer (50 mM ammonium acetate, 2 mM sodium azide, pH 5.5). Samples were loaded onto the columns, washed with 1.5 mL wash buffer (50 mM ammonium acetate in 50% acetonitrile, pH 5.5) three times. Samples were eluted with 0.6 mL elution buffer (10 mM EDTA, 100 mM ammonium bicarbonate, 10 mM DTT in 40% acetonitrile and 10% THF, pH 8.8) and dried using nitrogen flow (65 psi N₂ at 40° C.; TurboVap, Biotage). After SPE, samples were reconstituted in 120 μL water, they were analyzed using liquid chromatography combined with mass spectrometry (MS) detection on Thermo QExactive by electrospray ionization (ESI). Samples were injected (30 μL) and separated using an XBridge BEH C8 XP Column (130 Å, 2.5 μm, 2.1×30 mm; Waters, Cat. 176002554) using a gradient (mobile phase A: 16 mM triethylamine, 200 mM hexafluoroisopropanol; mobile phase B: methanol) of 0-65% mobile phase B over 6.2 minutes at a flowrate of 1 mL/min at 80° C. The ESI source was operated in negative ion mode, with full scan, using spray voltage=2800V, sheath gas flow=65 units, auxiliary gas flow=20 units, sweep gas flow=4 units, capillary temperature=300° C., and auxiliary gas heated to 300° C. Promass software was used to deconvolute the signal. Observed masses were mapped to metabolites and reported as percent of total strand.

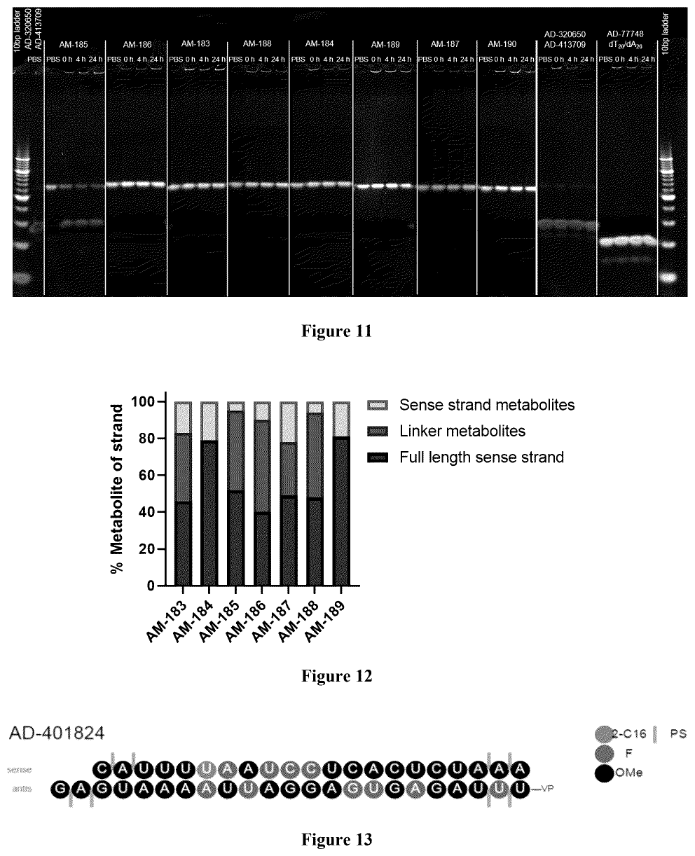

Degradation of bis-siRNA designs AM-183 to AM-190 in rat CSF was observed after 0, 4, or 24 h incubation or 24 h incubation in PBS as a control (FIG. 11). Compounds were analyzed via polyacrylamide gel electrophoresis to determine cleavability of the likers as described above. All nucleotide linkers evaluated were stable in rat CSF up to 24 h, apart from the RNA linker of AM-185, where the majority of it was cleaved after 24 h (FIG. 11).

The nature of sense strand metabolites observed after 24 h incubation of bis-siRNA designs AM-183 to AM-190 in rat brain homogenate was analyzed via MS, as described above, and is shown in FIG. 12. AM-184 and AM-189 containing 2′-OMe nucleotide linkers remained stable under these conditions. The remaining bis-siRNA with RNA or DNA linkers showed 30-50% cleaved product and some strand metabolism (FIG. 12).

### Example 6: Early Bis-siRNA Designs Showed Long-Term Distribution Between CSF and Plasma Akin to Parent siRNAs and Exhibited Drug Concentrations about Ten-Fold Higher in CSF than Plasma

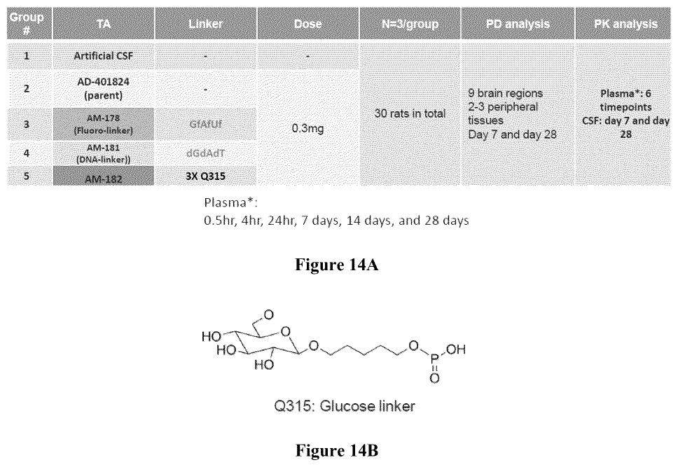

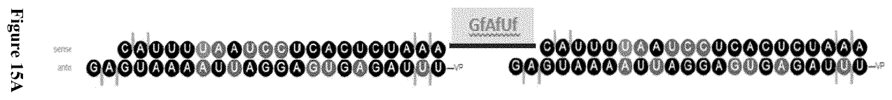

Initial CNS-targeting bis-siRNA designs employed the SOD1-targeting AD-401824 siRNA (FIG. 13) as parent siRNA. Bis-siRNA designs of the AD-401824 siRNA included two 5′-terminal antisense vinylphosphate groups, two C₁₆ lipophilic ligands (present at position 6 of each sense strand), twenty 2′-fluoro-modified nucleotides (with remaining siRNA nucleotides 2′-O-methyl-modified), and sixteen phosphorothioates in each bis-siRNA design. Relative to a completely symmetric design, an expanded scope of linkers—including nucleotide linkers—are available for the presently exemplfied bis-siRNA designs disclosed herein, which possess 3′-terminus-to-5′-terminus linkages of parent siRNA sense strands. Specific linkers selected for the early bis-siRNAs of the instant disclosure (FIG. 14A) included: a trinucleotide fluoro-linker (GfAfUf), described as a cytosol-cleaved design and used in the “AM-178” bis-siRNA design; a trinucleotide DNA-linker (dGdAdT), described as design predicted to partially cleave in cerebrospinal fluid (CSF) and used in the “AM-181” bis-siRNA design; and a triple “Q315” glucose linker (FIG. 14B), predicted to be cleaved in the endosome and used in the “AM-182” bis-siRNA design. To determine if variously linked “tail (3′-end)-to-head (5′-end)” bis-siRNA designs could impact pharmacokinetics (PK) and pharmacodynamics (PD) of component siRNAs in CNS, intrathecal (IT) dosing (at 0.3 mg) of rat cohorts was performed at to, and serum collection for PK analysis (via stem loop qPCR) was performed at 30 min, 4 hour, 24 hour, 7 day, 14 day, 21 day and 28 day time points (in total, 165 serum samples were used for PK analysis). Meanwhile, CNS brain tissues were harvested at day 7 and day 28 for PD analysis (in total, 360 brain/periphery tissues were harvested for PD analysis).

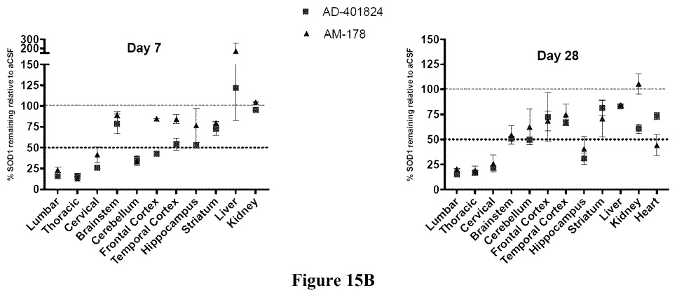

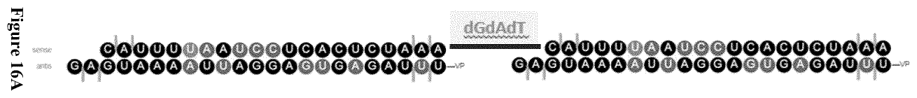

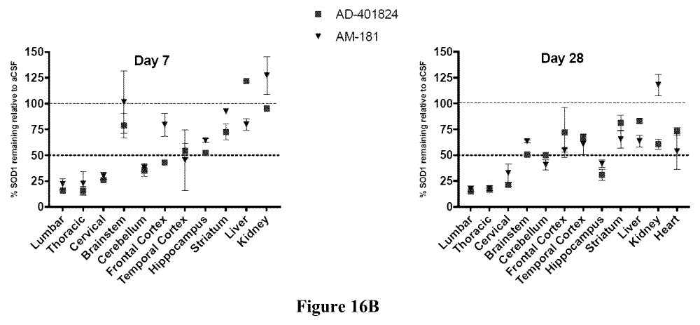

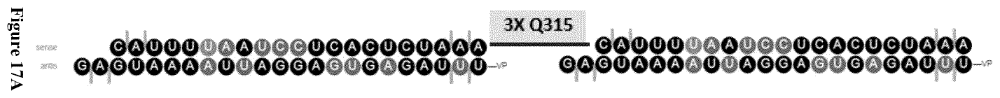

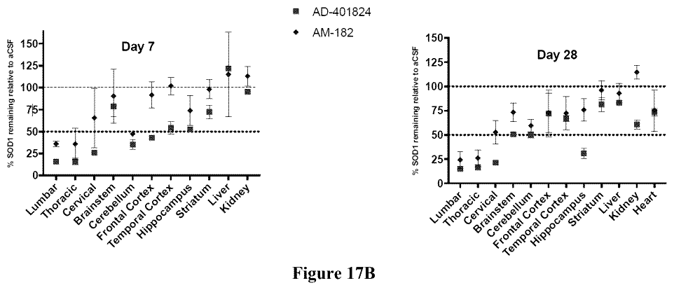

The fluoro-linked “AM-178” bis-siRNA design exhibited comparable knockdown across tissues at day 7 and day 28 (FIGS. 15A and 15B). The DNA-linked “AM-181” bis-siRNA design also exhibited comparable knockdown across tissues at day 7 and day 28 (FIGS. 16A and 16B). In contrast, the triple “Q315”-linked “AM-182” bis-siRNA design exhibited less favorable knockdown across tissues at day 7 and day 28 (FIGS. 17A and 17B). Thus, for these early bis-siRNA designs, either comparable or less favorable levels of knockdown relative to parent siRNAs were observed across CNS tissues on day 7 and day 28. It was considered that longer duration studies would be needed to assess whether the bis-siRNA designs might exhibit more sustainable knockdown.

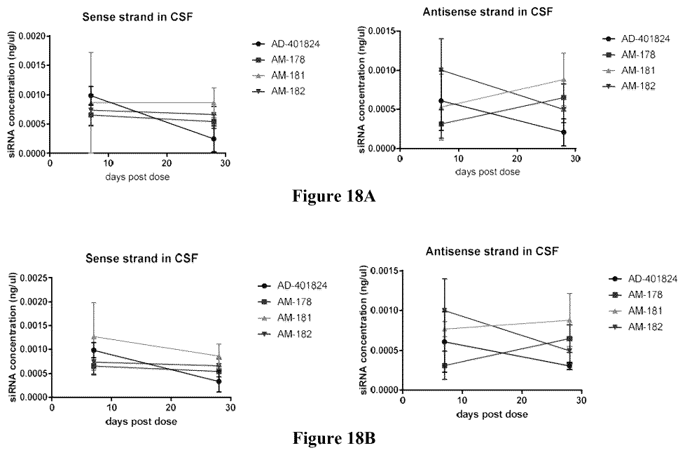

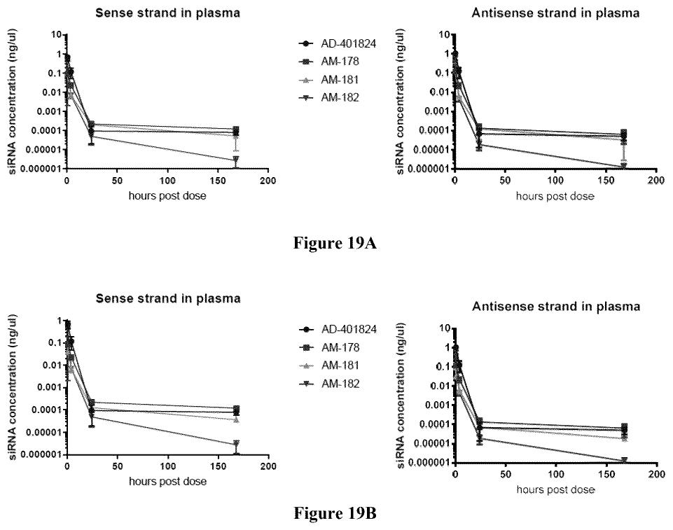

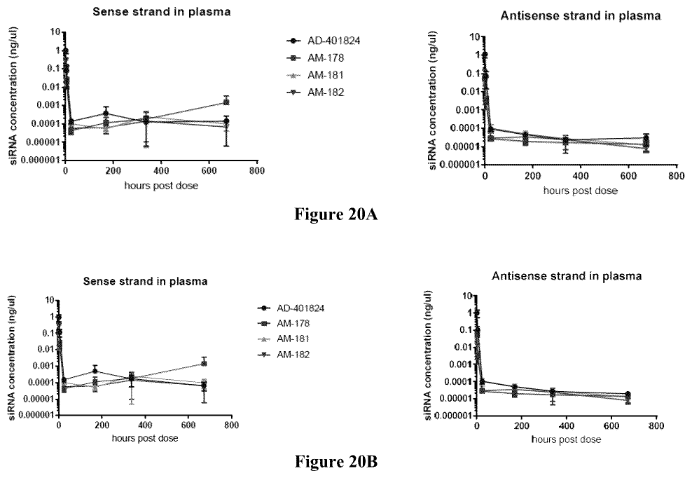

Delivered levels of parent siRNA and bis-siRNA designs in terminal CSF were assessed at day 7 and day 28 (FIGS. 18A and 18B, where FIG. 18B shows a chart that has two animals removed, as compared to FIG. 18A: animal #11 (day 7 AM-181) and #19 (day 28 AD-401824 parent siRNA); it is also noted that no CSF was obtained from animal #30, a 28 day AM-182-dosed animal), and no significant differences in CSF concentration of dosed agents was observed. Levels of parent siRNA and bis-siRNA designs in plasma were also assessed at day 7 (FIGS. 19A and 19B, where lower levels of AM-182 were observed at day 7, noting that FIG. 19B removes a single animal (#11, AM-181 day 7) from the analysis of FIG. 19A) and day 28 (FIGS. 20A and 20B, where no significant differences were observed in long-term pharmacokinetics out to day 28, noting that FIG. 20B removes a single animal (#19, AD-401824 day 28) from the analysis of FIG. 20A).

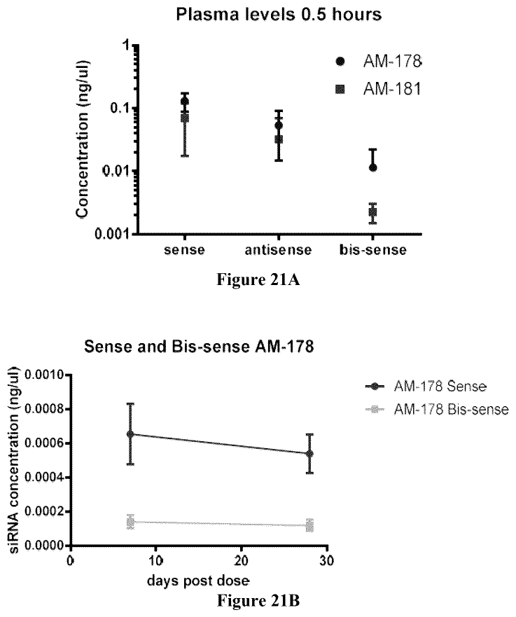

Quantification of bis-siRNA construct sense strands was also performed. To assess bis structures only, bis-specific primers were designed, which anneal to the nucleotide linker region of bis constructs and amplify only the bis-sense strand. Bis-specific primers were, of course, only viable with nucleotide-linked structures (i.e., AM-178 and AM-181) and were not compatible with AM-182 (as the glucose linker was not compatible with primer design). As expected, no amplification was observed with AD-401824. Notably, the exact quantification of bis-sense strand was difficult due to low signal, and caution should therefore be exercised when comparing to sense strand to determine percent of the bis construct that was cleaved. After IT injection, intact AM-178 and AM-181 bis-siRNAs were detected in plasma at 30 min post-dose (FIG. 21A). Thus, bis-siRNA design did not not prevent leakage into plasma. Meanwhile, CSF levels (day 7 and day 28) and later plasma timepoints were below the lower limit of quantitation (LLOQ), due to low signal. About three-fold higher amounts of intact AM-178 than AM-181 were detected, despite similar levels of total drug. Such observations were consistent with faster cleavage of the DNA linker of AM-181 (per in vitro data). The data presented in FIG. 21A therefore indicated that 30 minutes post-dose in plasma: approximately 10-20% of AM-178 (Fluoro) linker remained intact, while approximately 3-6% of AM-181 (DNA) linker remained intact.

Bis-sense strand quantification also revealed that intact AM-178, but not AM-181, was detected in CSF at day 7 and day 28 (FIG. 21B). This result was consistent with more rapid cleavage of the DNA linker of AM-181, while the results also indicated that approximately 20% of the AM-178 linker constructs remained intact in CSF at day 7 and 28.

To summarize the pharmacokinetic (PK) analysis of bis-SOD1 designs versus parent siRNA AD-401824, bis-siRNA designs were identified not to significantly impact long-term distribution between CSF and plasma. While higher plasma levels of parent siRNA AD-401824 were observed within hours of dosing, this initially observed difference was negligible by 24 hours post-dose. Meanwhile, lower levels of AM-182 were observed at day 7 in plasma in the day 7 set, yet these results were not repeated in the day 28 set. Notably, these results generally correlated with observations of decreased efficacy of AM-182 in several tissues. While intact AM-178 and AM-181 bis-siRNAs were detected in plasma at 30 min post-dose, concentrations of these agents were approximately 10-fold higher in CSF than in plasma at day 7 and day 28.

Thus, the DNA linker of AM-181 was likely cleaved more rapidly than the fluoro linker of AM-178. A roughly 3-fold higher amount of intact AM-178 than AM-181 was observed in plasma (at 30 min), while only intact AM-178 was detected in CSF (levels of AM-181 were below the LLOQ). A similar ratio of intact versus cleaved AM-178 was also observed in plasma and CSF, consistent with concluding that bis design did not impact retention of bis-siRNAs in the CSF.

TABLE 1
Abbreviations of nucleotide monomers used
in nucleic acid sequence representation.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | 5′-(E)-Vinylphosphonate |
| (Aam) | 2′-O-(N-methylacetamide)adenosine-3′-phosphate |
| (Aams) | 2-O-(N-methylacetamide)adenosine-3′-phosphorothioate |
| (Gam) | 2-O-(N-methylacetamide)guanosine-3′-phosphate |
| (Gams) | 2′-O-(N-methylacetamide)guanosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (Aeo) | 2′-O-methoxyethyladenosine-3′-phosphate |
| (Aeos) | 2-O-methoxyethyladenosine-3′-phosphorothioate |
| (Geo) | 2′-O-methoxyethylguanosine-3′-phosphate |
| (Geos) | 2-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2-O-methoxyethyl-5-methylcytidine-3′-phosphorothioate |
| (A3m) | 3′-O-methyladenosine-2′-phosphate |
| (A3mx) | 3′-O-methyl-xylofuranosyladenosine-2′-phosphate |
| (G3m) | 3′-O-methylguanosine-2′-phosphate |
| (G3mx) | 3-O-methyl-xylofuranosylguanosine-2′-phosphate |
| (C3m) | 3′-O-methylcytidine-2′-phosphate |
| (C3mx) | 3′-O-methyl-xylofuranosylcytidine-2′-phosphate |
| (U3m) | 3′-O-methyluridine-2′-phosphate |
| (U3mx) | 3′-O-methyl-xylofuranosyluridine-2′-phosphate |
| (m5Cam) | 2′-O-(N-methylacetamide)-5-methylcytidine-3′-phosphate |
| (m5Cams) | 2-O-(N-methylacetamide)-5-methylcytidine-3′-phosphorothioate |
| (Ahd) | 2′-O-hexadecyl-adenosine-3′-phosphate |
| (Ahds) | 2′-O-hexadecyl-adenosine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |

It will be understood that these monomers, when present in an oligonucleotide, are mutually linked by 5′-3′-phosphodiester bonds; and it is understood that when the nucleotide contains a 2′-fluoro modification, then the fluoro replaces the hydroxy at that position in the parent nucleotide (i.e., it is a 2′-deoxy-2′-fluoronucleotide).

TABLE 2
Sequences of Single Strands Synthesized for Bis-siRNA′s and Their Controls (with
nucleotide modifications)

| | | | | | SEQ |
|---|---|---|---|---|---|
| | Duplex | Sequence | Strand | | ID |
| Target | ID | ID | (S/AS) | Sequence (5′ to 3′) | NO: |
| SOD1 | AD- | A-555737 | S | csasuuu(Uhd)AfaUfCfCfucacucuasasa | 13 |
| | 413709 | | | | |
| SOD1 | AD- | A-637451 | AS | VPuUfuagAfgUfGfaggaUfuAfaaaugsasg | 14 |
| | 413709 | | | | |
| CTNNB1 | AD- | A-637448 | S | usascug(Uhd)UfgGfAfUfugauucgasasa | 15 |
| | 320650 | | | | |
| CTNNB1 | AD- | A-141362 | AS | VPuUfucgAfaUfCfaaucCfaAfcaguasgsc | 16 |
| | 320650 | | | | |
| CTNNB1/ | AM-183 | A- | S | usascug(Uhd)UfgGfAfUfugauucgasasad | 17 |
| SOD1 | | 2221110.1 | | TdTdTcsasuuu(Uhd)AfaUfCfCfucacucu | |
| | | | | asasa | |
| CTNNB1/ | AM-184 | A- | S | usascug(Uhd)UfgGfAfUfugauucgasasau | 18 |
| SOD1 | | 2221111.1 | | uucsasuuu(Uhd)AfaUfCfCfucacucuasasa | |
| CTNNB1/ | AM-185 | A- | S | usascug(Uhd)UfgGfAfUfugauucgasasaU | 19 |
| SOD1 | | 2221112.1 | | UUcsasuuu(Uhd)AfaUfCfCfucacucuasas | |
| | | | | a | |
| CTNNB1/ | AM-186 | A- | S | usascug(Uhd)UfgGfAfUfugauucgasasaU | 20 |
| SOD1 | | 2221113.1 | | fUfUfcsasuuu(Uhd)AfaUfCfCfucacucua | |
| | | | | sasa | |
| CTNNB1/ | AM-187 | A- | S | usascuguUfgGfAfUfugauucgasasadTdTd | 21 |
| SOD1 | | 2112117.1 | | Tcsasuuu(Uhd)AfaUfCfCfucacucuasasa | |
| SOD1/ | AM-188 | A- | S | csasuuu(Uhd)AfaUfCfCfucacucuasasadT | 22 |
| CTNNB1 | | 2112118.1 | | dTdTusascug(Uhd)UfgGfAfUfugauucga | |
| | | | | sasa | |
| SOD1/ | AM-189 | A- | S | csasuuu(Uhd)AfaUfCfCfucacucuasasauu | 23 |
| CTNNB1 | | 2112119.1 | | uusascug(Uhd)UfgGfAfUfugauucgasasa | |
| CTNNB1/ | AM-190 | A- | S | usascug(Uhd)UfgGfAfUfugauucgasasad | 24 |
| SOD1 | | 2112130.1 | | TdTdTcsasuuuuAfaUfCfCfucacucuasasa | |
| CONTROL | AD- | A-110415 | S | dTdTdTdTdTdTdTdTdTdTdTdTdTdTd | 25 |
| | 77748 | | | TdTdTdTdTdT | |
| CONTROL | AD- | A-156352 | AS | dAdAdAdAdAdAdAdAdAdAdAdAdAd | 26 |
| | 77748 | | | AdAdAdAdAdAdA | |

TABLE 3
Sequences of Single Strands Synthesized for Bis-siRNA′s and Their Controls
(without nucleotide modifications)

| | | | | | SEQ |
|---|---|---|---|---|---|
| | Duplex | Sequence | Strand | | ID |
| Target | ID | ID | (S/AS) | Sequence (5′ to 3′) | NO: |
| SOD1 | AD- | A-555737 | S | CAUUUUAAUCCUCACUCUAAA | 1 |
| | 413709 | | | | |
| SOD1 | AD- | A-637451 | AS | UUUAGAGUGAGGAUUAAAAUG | 2 |
| | 413709 | | | AG | |
| CTNNB1 | AD- | A-637448 | S | UACUGUUGGAUUGAUUCGAAA | 3 |
| | 320650 | | | | |
| CTNNB1 | AD- | A-141362 | AS | UUUCGAAUCAAUCCAACAGUAG | 4 |
| | 320650 | | | C | |
| CTNNB1/ | AM- | A- | S | UACUGUUGGAUUGAUUCGAAAT | 5 |
| SOD1 | 183 | 2221110.1 | | TTCAUUUUAAUCCUCACUCUAA | |
| | | | | A | |
| CTNNB1/ | AM- | A- | S | UACUGUUGGAUUGAUUCGAAAU | 6 |
| SOD1 | 184 | 2221111.1 | | UUCAUUUUAAUCCUCACUCUAA | |
| | | | | A | |
| CTNNB1/ | AM- | A- | S | UACUGUUGGAUUGAUUCGAAAU | 7 |
| SOD1 | 185 | 2221112.1 | | UUCAUUUUAAUCCUCACUCUAA | |
| | | | | A | |
| CTNNB1/ | AM- | A- | S | UACUGUUGGAUUGAUUCGAAAU | 8 |
| SOD1 | 186 | 2221113.1 | | UUCAUUUUAAUCCUCACUCUAA | |
| | | | | A | |
| CTNNB1/ | AM- | A- | S | UACUGUUGGAUUGAUUCGAAAT | 9 |
| SOD1 | 187 | 2112117.1 | | TTCAUUUUAAUCCUCACUCUAA | |
| | | | | A | |
| SOD1/ | AM- | A- | S | CAUUUUAAUCCUCACUCUAAAT | 10 |
| CTNNB1 | 188 | 2112118.1 | | TTUACUGUUGGAUUGAUUCGAA | |
| | | | | A | |
| SOD1/ | AM- | A- | S | CAUUUUAAUCCUCACUCUAAAU | 11 |
| CTNNB1 | 189 | 2112119.1 | | UUUACUGUUGGAUUGAUUCGAA | |
| | | | | A | |
| CTNNB1/ | AM- | A- | S | UACUGUUGGAUUGAUUCGAAAT | 12 |
| SOD1 | 190 | 2112130.1 | | TTCAUUUUAAUCCUCACUCUAA | |
| | | | | A | |
| CONTROL | AD- | A-110415 | S | TTTTTTTTTTTTTTTTTTTT | 27 |
| | 77748 | | | | |
| CONTROL | AD- | A-156352 | AS | AAAAAAAAAAAAAAAAAAAA | 28 |
| | 77748 | | | | |

TABLE 4
Parent SOD1-Targeting siRNA Sequences

| | | | | | SEQ | | SEQ |
|---|---|---|---|---|---|---|---|
| Duplex | Oligo | | | | ID | | ID |
| ID | ID | Strand | Target | Oligo Seq (5′-3′) | NO: | Trans Seq (5′-3′) | NO: |
| AD- | A- | sense | SOD1 | csasuuu(Uhd)AfaU | 29 | CAUUUUAAUCC | 30 |
| 401824 | 637448 | | | fCfCfucacucuasasa | | UCACUCUAAA | |
| | A- | antis | SOD1 | VPusUfsuagAfgUf | 31 | UUUAGAGUGAG | 32 |
| | 444402 | | | GfaggaUfuAfaaaug | | GAUUAAAAUGA | |
| | | | | sasg | | G | |

TABLE 5
Bis-siRNA Sequences of Example 6

| | | | | SEQ |
|---|---|---|---|---|
| Multiplex | | | | ID |
| Name | Strand | Target | Sequence | NO: |
| AM-178 | sense | SOD1/ | csasuuu(Uhd)AfaUfCfCfucacucuasasaGfAfUfcsasuuu | 33 |
| | | SOD1 | (Uhd)AfaUfCfCfucacucuasasa | |
| AM-181 | sense | SOD1/ | csasuuu(Uhd)AfaUfCfCfucacucuasasadGdAdTcsasuuu | 34 |
| | | SOD1 | (Uhd)AfaUfCfCfucacucuasasa | |
| AM-182 | sense | SOD1/ | csasuuu(Uhd)AfaUfCfCfucacucuasasaQ315Q315Q315csas | 35 |
| | | SOD1 | uuu(Uhd)AfaUfCfCfucacucuasasa | |

### Example 7: Evaluations of Knockdown in Mice of Bis-siRNA Complexes with the Individual Effector Molecules (siRNAs) Joined by a Nucleotide Linker

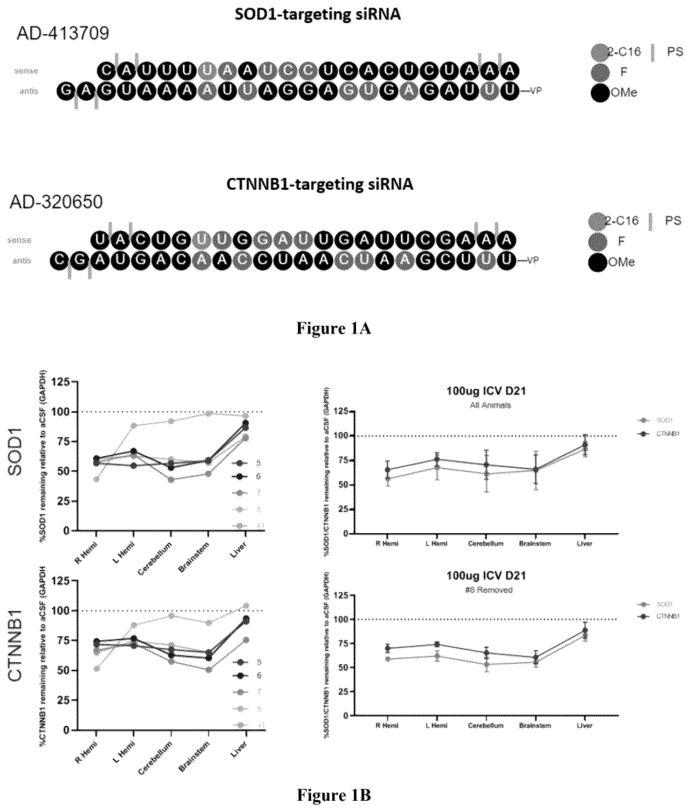

Multiple “bis” (dual-targeting) siRNA complexes were prepared, using synthesis methods previously described in PCT application no. PCT/US2016/042498. The “bis” siRNA complexes were designed for simultaneous (and stoichiometrically equivalent with respect to effector molecule concentration) inhibition of CTNNB1 and SOD1 nucleic acid targets (FIG. 1A and FIGS. 2A-2C). Each bis-siRNA complex included a continuous sense strand that respectively included a sense strand sequence within a first target nucleic acid (CTNNB1 or SOD1 mRNA) and a sense strand sequence within a second target nucleic acid (the other of CTNNB1 or SOD1 mRNA), the respective sense strands of the individual effector molecules (siRNAs) joined by a nucleotide linker of three nucleotides in length (as presently exemplified), and two separate and independent single-stranded antisense sequences that respectively target the first target nucleic acid and the second target nucleic acid and respectively anneal to complementary sense strand sequences of the continuous sense strand of the bis siRNA complex (FIG. 2D), with an exemplary bis siRNA complex (multi-targeted molecule) shown in FIG. 3A.

The sequences, chemical patterns, and linkers of of these bis-siRNA complexes are also listed in Tables 2-5.

The efficacy and tissue distribution of each bis siRNA (or mixed duplex control) was evaluated in the central nervous system and in the periphery of C57/BI6 mice. Single ICV administrations to the right ventricle of the mouse brain were performed using a 3.5 mm custom needle with a 25 μl Hamilton syringe, using isoflurane as anesthesia. 50 μg per 5 μl of each duplex was administered in the mixture duplex control. 100 μg per 5 μl of each of the bis-siRNA complexes was administered. All injections were performed by the same administrator. At least 4 animals were used per test compound. Animals were euthanized on day 21 post-administration. Inhibition of CTNNB1 and SOD1 was evaluated in the right and left hemispheres of the brain, cerebellum, brain stem, and liver.

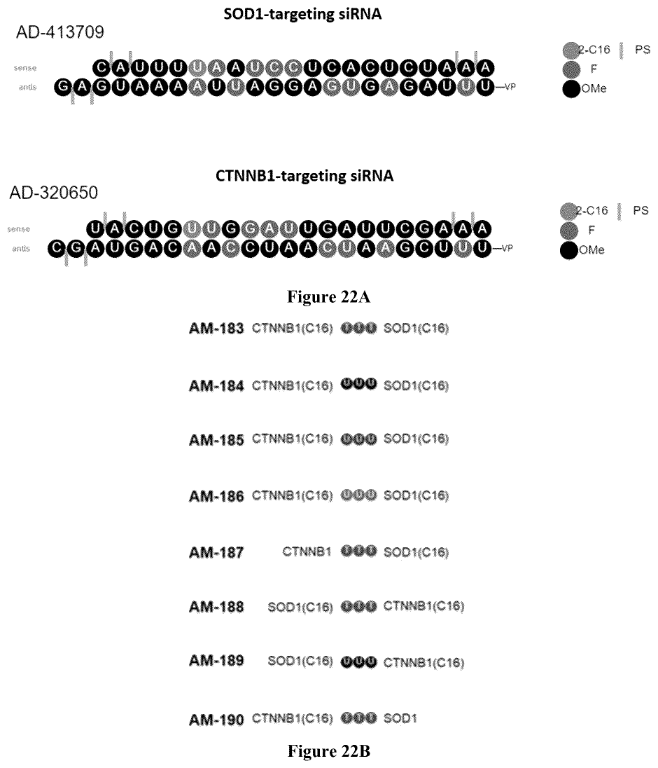

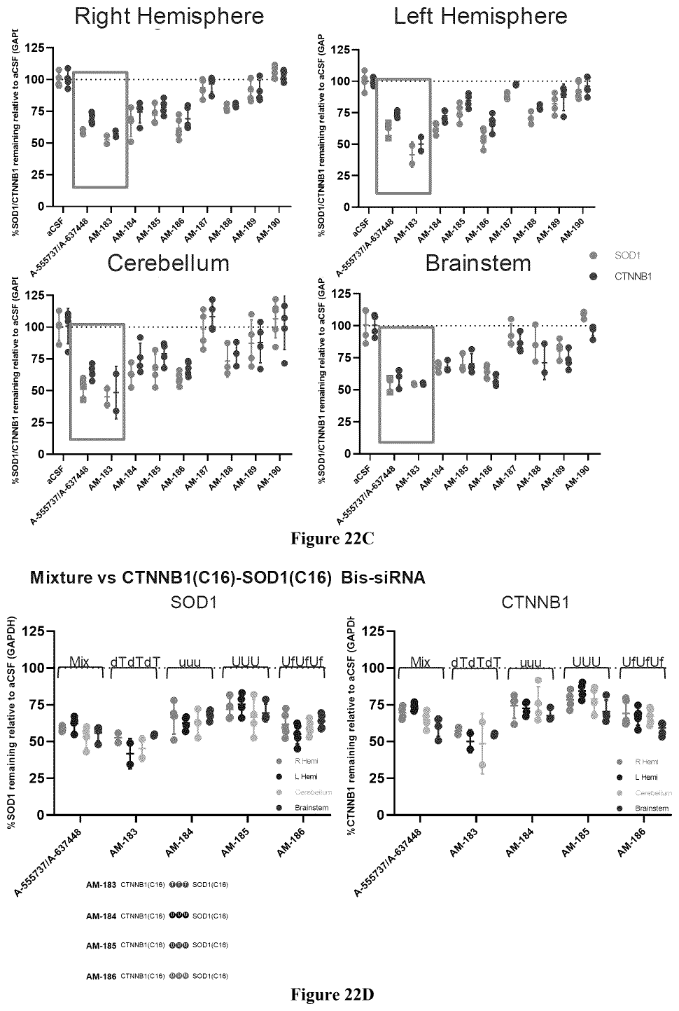

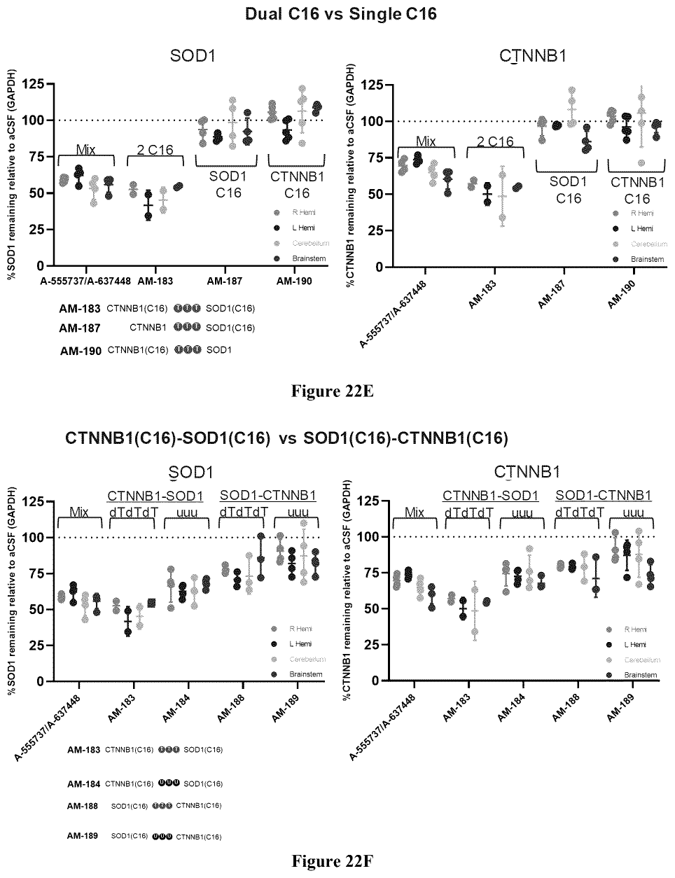

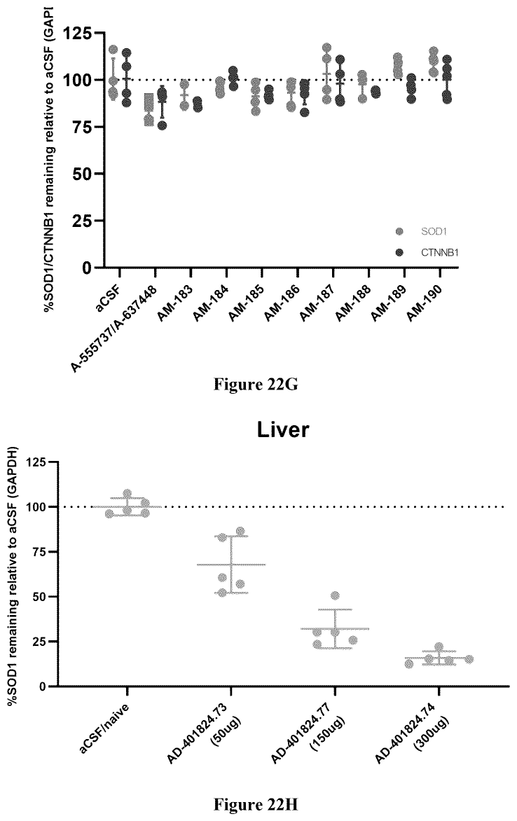

The knockdown of the target mSOD1 and mCTNNB1 by the bis-siRNA complexes were assessed in mice in the central nervous system constructed using respective C₁₆-modified SOD1- and CTNNB1-targeting siRNA molecules, possessing various sense-strand-joining nucleotide linkers, as tabulated in FIG. 2A and FIGS. 22A-22B. The target mRNA knockdown levels were also compared to mixed duplex delivery (mixture of siRNA of AD-413709, targeting SOD1, and siRNA of AD-320650, targeting CTTNB1, as shown in FIG. 2A and Table 2). Tissues collected for knock down assessment were the right hemisphere of the brain, left hemisphere of the brain, cerebellum, and brainstem.

The results are summarized in FIGS. 22C-22F.

FIG. 22C shows, for each individual mouse dosed, the percentage of SOD1 and CTNNB1 remaining at day 21 after a 100 μg ICV injection of each of the CTNNB1(C16)-SOD1(C16) bis siRNA molecules, in each of the indicated tissues. As shown in FIG. 22C, compared to both mixed duplex and other bis-siRNA linkers, the most effective bis siRNA linker construct observed for inhibition of both SOD1 and CTNNB1 in the CNS was identified as the dTdTdT-linked CTNNB1(C16)-SOD1(C16) bis-siRNA (AM-183), which exhibited an inhibition of about 50% or more for both SOD1 and CTNNB1 across all brain tissues examined.

FIG. 22D provides a more detailed illustration comparing some CTNNB1(C16)-SOD1(C16) bis siRNA molecules (AM-183, AM-184, AM-185, and AM-186) against the mixed duplex delivery (mixture of siRNA of AD-413709, targeting SOD1, and siRNA of AD-320650, targeting CTTNB1). FIG. 22D shows that the bis siRNA with a nucleotide-linker construct (e.g., dTdTdT, uuu, UUU, UfUfUf) provided better or at least comparable inhibition of for both SOD1 and CTNNB1 across all brain tissues examined as compared to the mixed duplex. FIG. 22D also confirms that compared to both mixed duplex and other bis-siRNA linkers, the most effective bis siRNA linker construct observed for inhibition of both SOD1 and CTNNB1 in the CNS was identified as the dTdTdT-linked CTNNB1(C16)-SOD1(C16) bis-siRNA (AM-183), which exhibited an inhibition of about 50% or more for both SOD1 and CTNNB1 across all brain tissues examined.

FIG. 22E shows the results of the percentage of SOD1 and CTNNB1, respectively, remaining at day 21 after a 100 μg ICV injection in the mice in each of the indicated tissues, comparing the bis siRNA complex possessing a single C₁₆ modification at SOD1-targeting siRNA (SOD1-C16) or CTNNB1-targeting siRNA (CTNNB1-C16) against the bis siRNA complex possessing a dual C₁₆ modification at both SOD1-targeting siRNA and CTNNB1-targeting siRNA (2C16 or CTNNB1(C16)-SOD1(C16)), and against the mixed duplex delivery (mixture of siRNA of AD-413709, targeting SOD1, and siRNA of AD-320650, targeting CTTNB1). As shown in FIG. 22E, when the bis siRNA complex possessing a single C₁₆ modification at either a SOD1-targeting siRNA (SOD1-C16) or a CTNNB1-targeting siRNA (CTNNB1-C16) were used, neither bis-siRNA complex exhibited an inhibitory efficacy in any of the brain tissues examined. These results were in stark contrast to the inhibitory efficacy observed in vivo in CNS tissues for bis siRNA complexes possessing a dual C₁₆ modification at both SOD1-targeting siRNA and CTNNB1-targeting siRNA (2C16 or CTNNB1(C16)-SOD1(C16)).

FIG. 22F shows the results of the percentage of SOD1 and CTNNB1, respectively, remaining at day 21 after a 100 μg ICV injection in the mice in each of the indicated tissues, comparing various bis siRNA molecules (AM-183, AM-184, AM-188, and AM-189, as shown in FIG. 2A and Table 2) varying the positions of the respective siRNA effectors within the bis-siRNA complex. In contrast to the robust mSOD1 and mCTNNB1 inhibitory activity observed for CTNNB1(C16)-SOD1(C16) bis siRNA complex (particularly for the one with a DNA linker dTdTdT), a corresponding SOD1(C16)-CTNNB1(C16) bis siRNA complex exhibited a decreased inhibition, for both mSOD1 and mCTNNB1 target mRNAs.

The knockdown of the target mSOD1 and mCTNNB1 by the bis-siRNA complexes were also assessed in mice in the liver using the same bis-siRNA complexes as discussed above, for comparison against the knockdown in the CNS. The result is summarized in FIG. 22G, which shows, for each individual mouse dosed, the percentage of SOD1 and CTNNB1 remaining at day 21 after a 100 μg ICV injection of each of the CTNNB1(C16)-SOD1(C16) bis siRNA molecules, in the liver, for a cohort of 4 animals. As compared to the inhibitory efficacy observed in vivo in CNS tissues for bis siRNA complexes (FIGS. 22C-22F), a relative minimal inhibitory efficacy was observed in the liver tissues examined for the same bis-siRNA complexes.

### Example 8a: Evaluations of Knockdown in Mice of Bis-siRNA Complexes with the Individual Effector Molecules (siRNAs) Joined by a Carbohydrate-Based Linker as Compared to a Nucleotide Linker

The preparation and design of the “bis” (dual-targeting) siRNA complexes used in this example are similar to those described in Example 7 above, except that the linkers joining the respective sense strands of the individual effector molecules (siRNAs) in this example included carbohydrate-based linkers, as compared to the nucleotide linkers used in Example 7. This example compares the exemplary CTNNB1(C16)-SOD1(C16) bis siRNA multi-targeted molecules having respective siRNA effector molecule sense strands joined by a carbohydrate-based linker against those having respective siRNA effector molecule sense strands joined by a nucleotide-based linker.

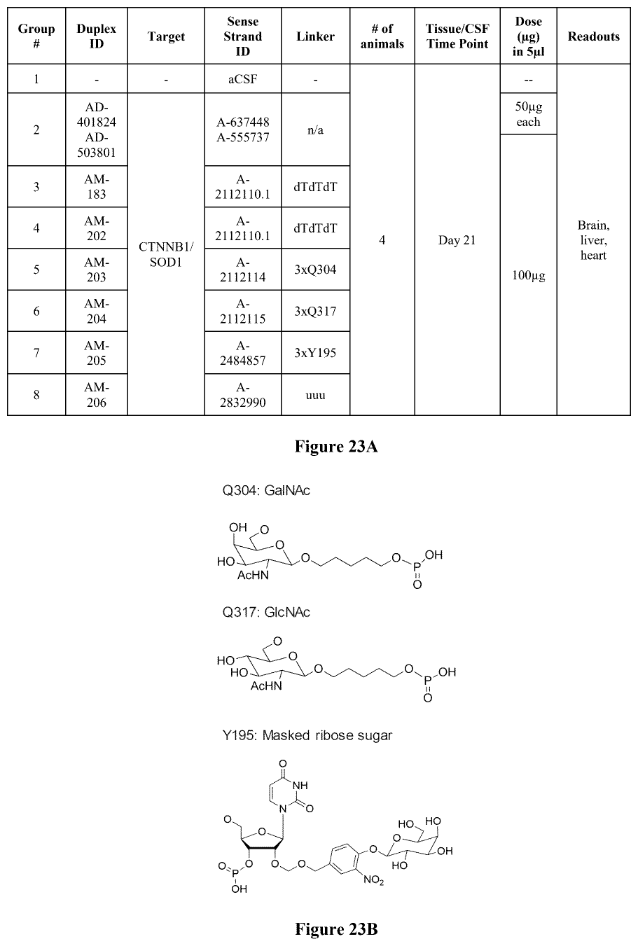

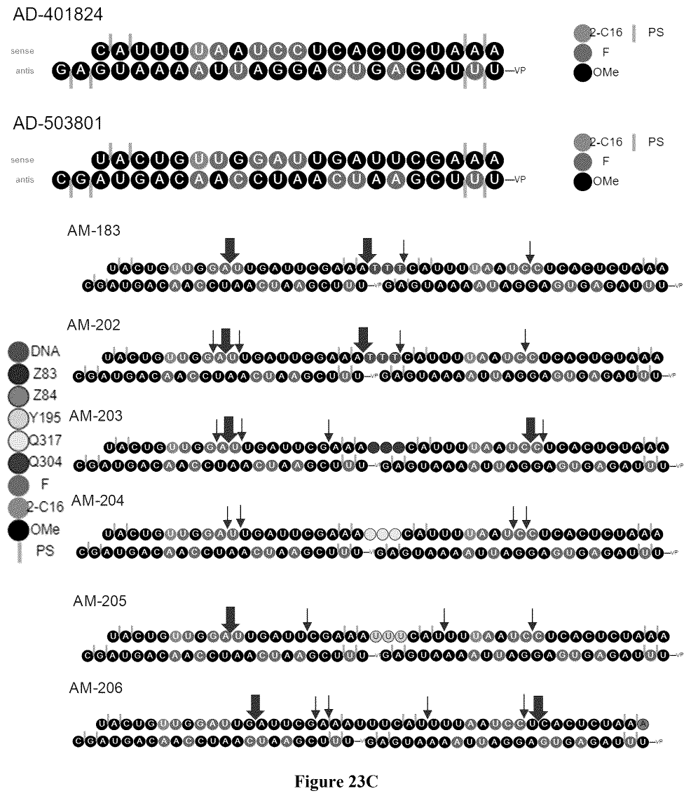

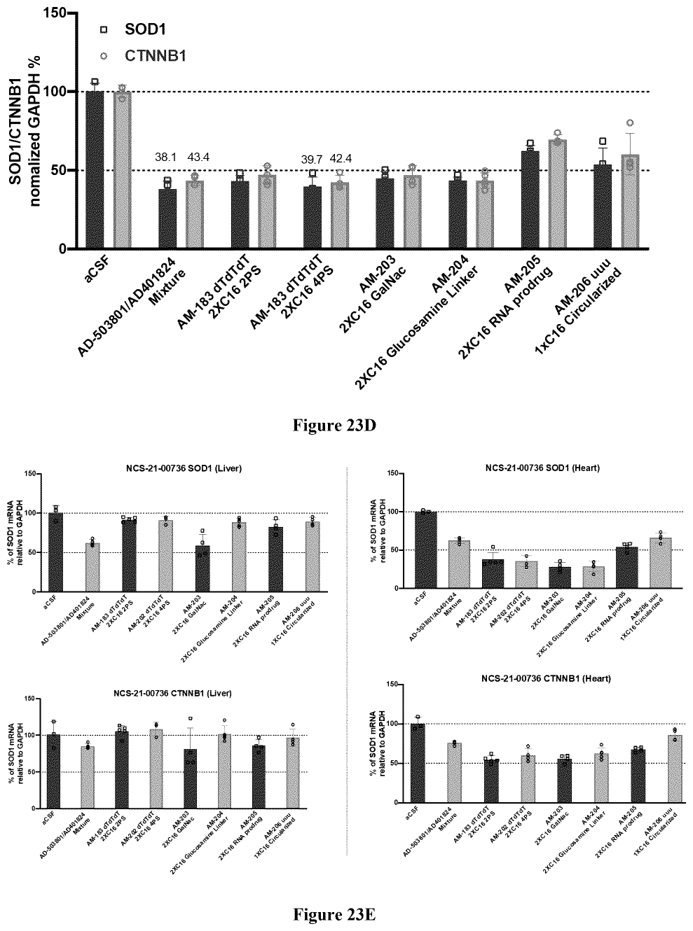

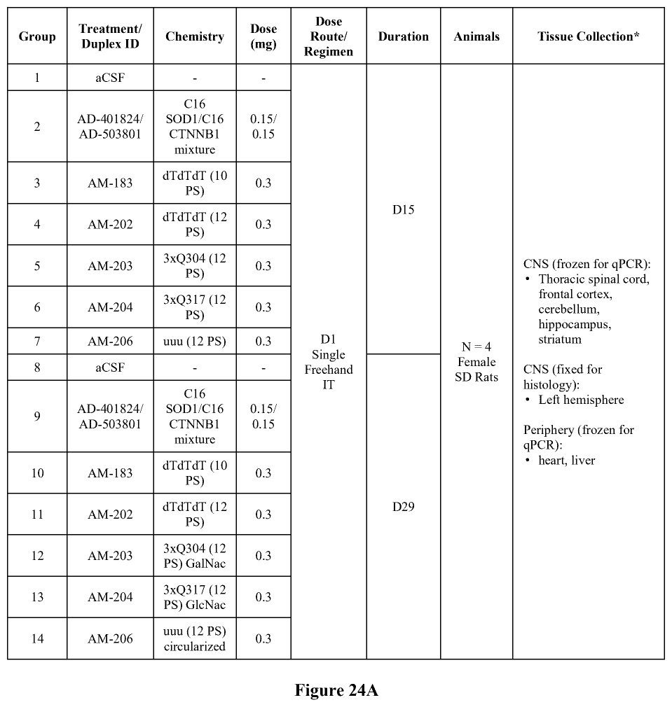

The sequences, chemical patterns, and linkers of these bis-siRNA complexes are shown in FIGS. 23A-23C.

The efficacy and tissue distribution of each bis siRNA (or mixed duplex control) was evaluated in the central nervous system and in the periphery of C57/BI6 mice. Single ICV administration to the mouse brain were performed with 50 μg per 5 μl of each duplex administered in the mixture duplex control, and 100 μg per 5 μl of each of the bis-siRNA complexes administered. At least 4 animals were used per test compound. Animals were euthanized on day 21 post-administration. Inhibition of CTNNB1 and SOD1 was evaluated in the brain, heart, and liver.

The knockdown of the target mSOD1 and mCTNNB1 by the bis-siRNA complexes were assessed in mice in the central nervous system constructed using respective C₁₆-modified SOD1- and CTNNB1-targeting siRNA molecules, possessing various sense-strand-joining nucleotide linkers, as tabulated in FIGS. 23A-23C. The target mRNA knockdown levels were also compared to mixed duplex delivery. Tissues collected for knock down assessment were brain, heart, and liver.

The results are summarized in FIGS. 23D-23E.

FIG. 23D compares the percentage of SOD1 and CTNNB1 in brain by using the bis siRNA complex possessing a three-carbohydrate linker (AM-203, AM204, AM205) against the bis siRNA complex possessing a three-nucleotide linker (AM183, AM202), and against the mixed duplex delivery. FIG. 23D shows that the bis siRNA complex possessing a three-carbohydrate linker had comparable inhibitions for both SOD1 and CTNNB1 in the CNS as compared to the bis siRNA complex possessing a three-nucleotide linker.

FIG. 23E compares the percentage of SOD1 and CTNNB1 in liver and heart by using the bis siRNA complex possessing a three-carbohydrate linker (AM-203, AM204, AM205) against the bis siRNA complex possessing a three-nucleotide linker (AM183, AM202), and against the mixed duplex delivery. FIG. 23E shows that the bis siRNA complex possessing a three-carbohydrate linker had comparable inhibitions for both SOD1 and CTNNB1 in heart as compared to the bis siRNA complex possessing a three-nucleotide linker. FIG. 23E also shows that both the bis siRNA complex possessing a three-carbohydrate linker and the bis siRNA complex possessing a three-nucleotide linker did not have a significant inhibitory efficacy for either SOD1 or CTNNB1 in liver.

### Example 8B: Evaluations of Knockdown in Mice of Bis-siRNA Complexes with the Individual Effector Molecules (siRNAs) Joined by a Nucleotide-Based Linker with a Circular Cyclized Sense Strand as Compared to a Nucleotide Linker with a Linear Sense Strand

FIG. 23D shows that AM-206, which has a cyclized circular sense strand containing only one C16 ligand, has a KD of about 50%, whereas AM-187 and AM-190, which have a bis siRNA having a linear sense strand and containing a single C16, has a lower observed degree of KD, as shown in FIG. 22E.

### Example 9: Evaluations of Knockdown in Rat Via Intrathecal (IT) Dosing of Bis-siRNA Complexes with the Individual Effector Molecules (siRNAs) Joined by Various Linkers and Having Various Chemistries

The preparation and design of the “bis” (dual-targeting) siRNA complexes used in this example are similar to those described in Example 8 above, except that the chemical modifications on the respective bis-siRNAs in this example containing various chemical modifications. This example evaluates the knockdown of the exemplary CTNNB1(C16)-SOD1(C16) bis siRNA multi-targeted molecules having respective siRNA effector molecule sense strands joined by various linkers and chemical modifications on the respective bis-siRNAs in rats via intrathecal injection.

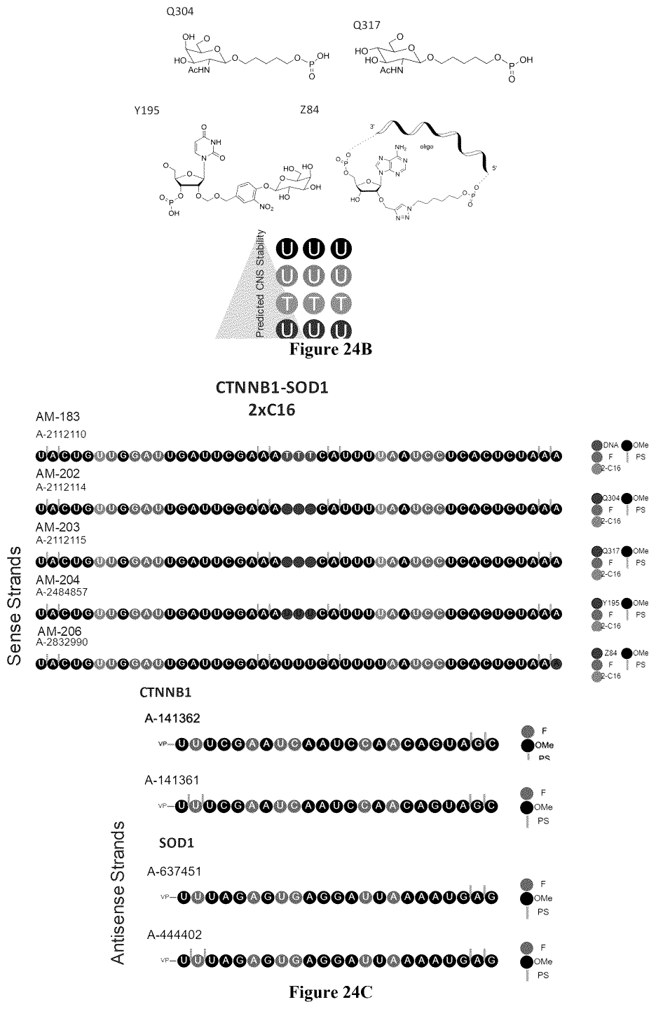

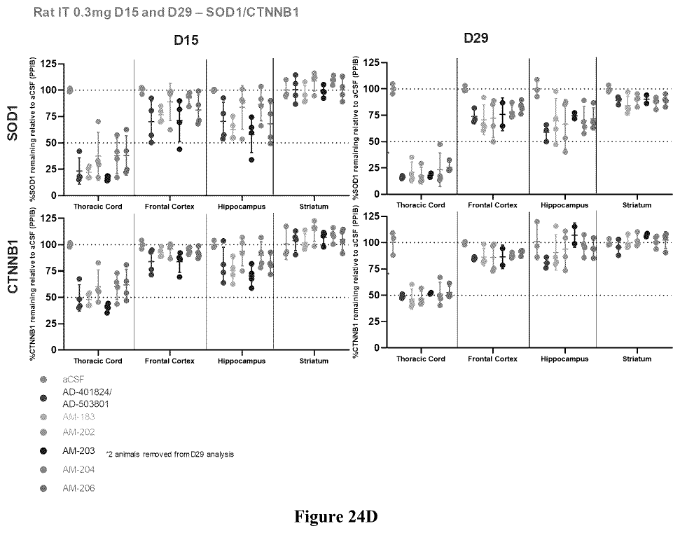

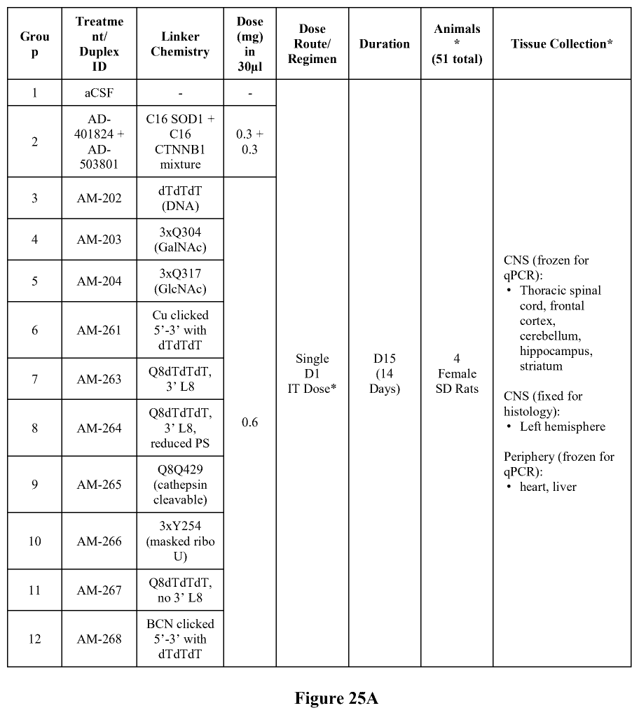

The sequences, chemical patterns, and linkers of these bis-siRNA complexes are shown in FIGS. 24A-24C.

The efficacy and tissue distribution of each bis siRNA (or mixed duplex control) was evaluated in the central nervous system. Single IT administration to the rat was performed with 0.15 mg of each duplex administered in the mixture duplex control, and 0.3 mg of each of the bis-siRNA complexes administered. At least 4 animals were used per test compound. The knockdown of the target mSOD1 and mCTNNB1 by the bis-siRNA complexes were assessed in rat in the central nervous system constructed using respective C₁₆-modified SOD1- and CTNNB1-targeting siRNA molecules, possessing various sense-strand-joining linkers and chemistries, as tabulated in FIGS. 24A-24C. The target mRNA knockdown levels also were compared to mixed duplex delivery. Tissues collected for knock down assessment were thoracic spinal cord, frontal cortex, hippocampus, and striatum.

The results are summarized in FIG. 24D.

FIG. 24D shows, for each individual mouse dosed, the percentage of SOD1 and CTNNB1 remaining at day 15 and day 29, respectively, after an intrathecal (IT) dosing (at 0.3 mg) of each of the CTNNB1(C16)-SOD1(C16) bis siRNA molecules (containing various linkers joining the respective sense strands of the individual effector molecules (siRNAs) and various chemical modifications in the bis-siRNA molecules, as shown in FIG. 24A-24C) was performed at to, in each of the indicated tissues (thoracic spinal cord, frontal cortex, hippocampus, and striatum), for a cohort of 4 animals, comparing against the mixed duplex delivery (mixture of siRNA of AD-401824, targeting SOD1, and siRNA of AD-503801, targeting CTTNB1, as shown in FIG. 23C).

FIG. 24D compares the percentage of SOD1 and CTNNB1 in various CNS tissues (thoracic spinal cord, frontal cortex, hippocampus, and striatum) by using the bis siRNA complex possessing various linkers having different chemistries. FIG. 24D suggests that all the exemplary bis siRNA complexes had comparable inhibitions for both SOD1 and CTNNB1 in respective CNS tissues. FIG. 24D also illustrates all the exemplary bis siRNA complexes appeared to have the most inhibitory efficary for either SOD1 or CTNNB1 in thoracic spinal cord, and no apparent inhibitions were observbed for either SOD1 or CTNNB1 in frontal cortex, hippocampus, and striatum.

### Example 10: Evaluations of Knockdown in Rat Via Intrathecal (IT) Dosing of Bis-siRNA Complexes with the Individual Effector Molecules (siRNAs) Joined by Various Linkers

The preparation and design of the “bis” (dual-targeting) siRNA complexes used in this example are similar to those described in Example 8 above, except that a wide variety of linkers were used to respective siRNA effector molecule sense strands. This example evaluates the knockdown of the exemplary CTNNB1(C16)-SOD1(C16) bis siRNA multi-targeted molecules having respective siRNA effector molecule sense strands joined by various linkers in rats via IT injection.

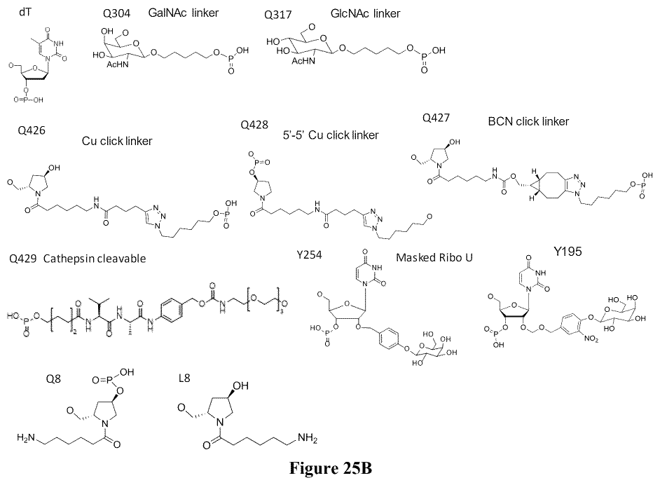

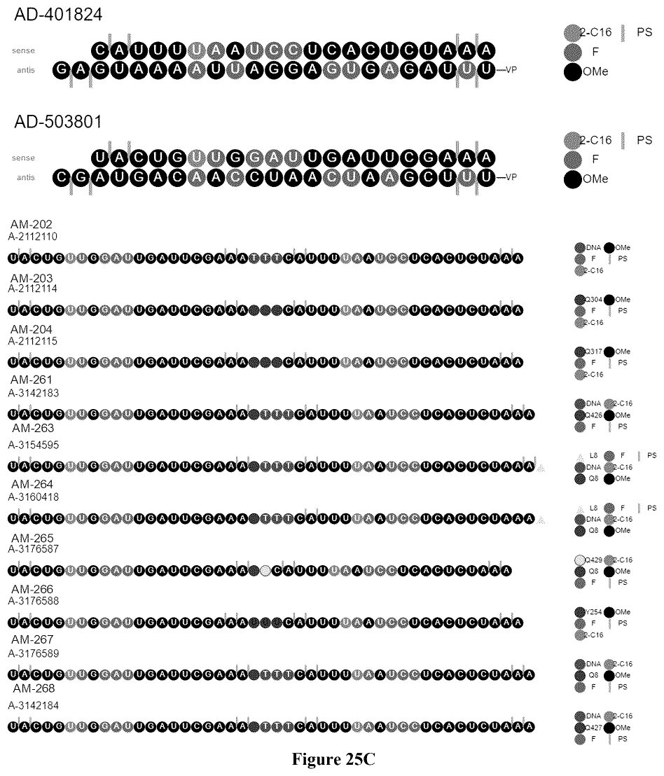

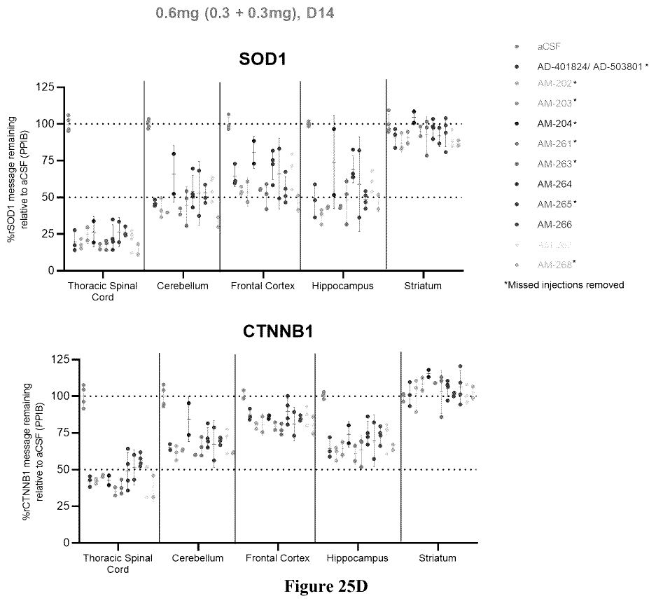

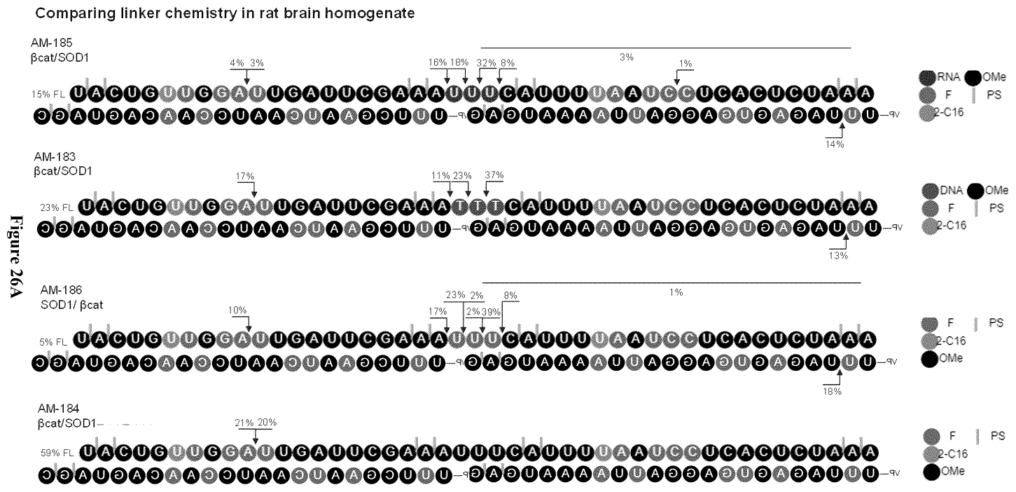

The sequences, chemical patterns, and linkers of these bis-siRNA complexes are shown in FIGS. 25A-25C.

The efficacy and tissue distribution of each bis siRNA (or mixed duplex control) was evaluated in the central nervous system. Single IT administration to the rat was performed with 0.3 mg of each duplex administered in the mixture duplex control, and 0.6 mg of each of the bis-siRNA complexes administered. At least 4 animals were used per test compound. The knockdown of the target mSOD1 and mCTNNB1 by the bis-siRNA complexes were assessed in rat in the central nervous system constructed using respective C₁₆-modified SOD1- and CTNNB1-targeting siRNA molecules, possessing various sense-strand-joining linkers, as tabulated in FIGS. 25A-25C. The target mRNA knockdown levels also were compared to mixed duplex delivery. Tissues collected for knock down assessment were thoracic spinal cord, cerebellum, frontal cortex, hippocampus, and striatum.

The results are summarized in FIG. 25D. FIG. 25D compares the percentage of SOD1 and CTNNB1 in various CNS tissues (thoracic spinal cord, cerebellum, frontal cortex, hippocampus, and striatum) by using the bis siRNA complex possessing a wide variety of linkers. FIG. 25D suggests that all the exemplary bis siRNA complexes had comparable inhibitions for both SOD1 and CTNNB1 in respective CNS tissues. FIG. 25D also illustrates all the exemplary bis siRNA complexes appeared to have the most inhibitory efficary for either SOD1 or CTNNB1 in thoracic spinal cord; all the exemplary bis siRNA complexes appeared to have significant inhibitory efficary for either SOD1 or CTNNB1 in cerebellum. Compared to FIG. 24D, the dosage has increased from 0.3 mg (FIG. 24D) to 0.6 mg (FIG. 25D), and this increased dosage appeared to have resulted in an improved inhibition for either SOD1 or CTNNB1 in frontal cortex and hippocampus. Even at the increased dosage at 0.6 mg, there was no apparent inhibition observbed for either SOD1 or CTNNB1 in striatum.

### Example 11: Analysis of Oligonucleotide Stability/Metabolism of the Bis-siRNA Complexes in Rat Brain Homogenates

Stability of bis-siRNAs were evaluated in a custom premium formulation prepared from female Sprague-Dawley rat whole brain tissue (BioIVT, Cat. S05966) formulated as Ig tissue per 3 mL buffer (50 mM Tris HCl, 1% NP-40, 140 mM NaCl with cOmplete EDTA-free Protease Inhibitor Cocktail (Sigma Aldrich, Cat. 11836170001), pH to 7.4), and the metabolized were analyzed via high-accuracy high-resolution mass spectrometry. Bis-siRNAs (˜8 μM) were incubated with brain at 1:50 ratio for 0 or 24 hours. The mixture was diluted with 450 μL lysis buffer (Phenomenex, Cat. AL0-8579) and solid phase extraction (SPE) was performed using Clarity OTX solid phase extraction plates (Phenomenex, Cat. 8E-5103-EGA) with positive pressure facilitated by ExtraHera (Biotage) instrument. Columns were conditioned with 1 mL methanol, followed by 1.9 mL equilibration buffer (50 mM ammonium acetate, 2 mM sodium azide, pH 5.5). Samples were loaded onto the columns, washed with 1.5 mL wash buffer (50 mM ammonium acetate in 50% acetonitrile, pH 5.5) three times. Samples were eluted with 0.6 mL elution buffer (10 mM EDTA, 100 mM ammonium bicarbonate, 10 mM DTT in 40% acetonitrile and 10% THF, pH 8.8) and dried using nitrogen flow (65 psi N₂ at 40° C.; TurboVap, Biotage). After SPE, samples were reconstituted in 120 μL water, they were analyzed using liquid chromatography combined with mass spectrometry (MS) detection on Thermo QExactive by electrospray ionization (ESI). Samples were injected (30 μL) and separated using an XBridge BEH C8 XP Column (130 Å, 2.5 μm, 2.1×30 mm; Waters, Cat. 176002554) using a gradient (mobile phase A: 16 mM triethylamine, 200 mM hexafluoroisopropanol; mobile phase B: methanol) of 0-65% mobile phase B over 6.2 minutes at a flowrate of 1 mL/min at 80° C. The ESI source was operated in negative ion mode, with full scan, using spray voltage=2800V, sheath gas flow=65 units, auxiliary gas flow=20 units, sweep gas flow=4 units, capillary temperature=300° C., and auxiliary gas heated to 300° C. Promass software was used to deconvolute the signal. Observed masses were mapped to metabolites and reported as percent of total strand.

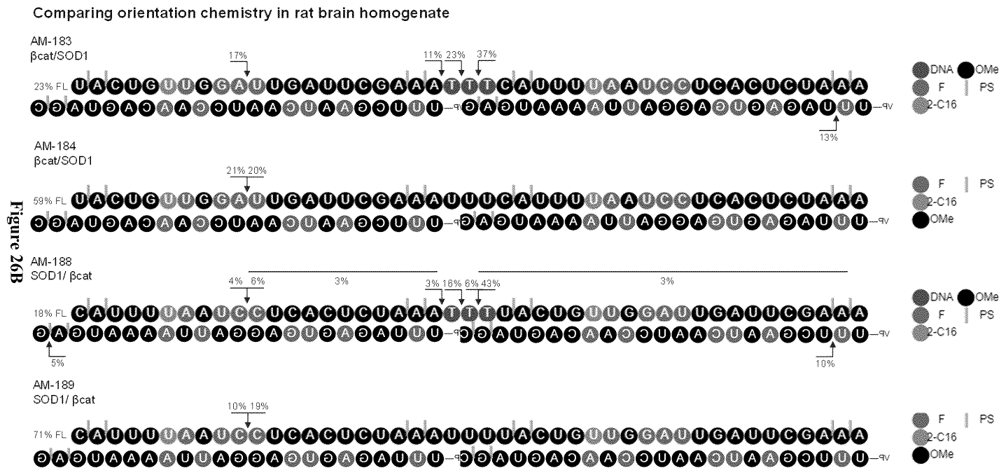

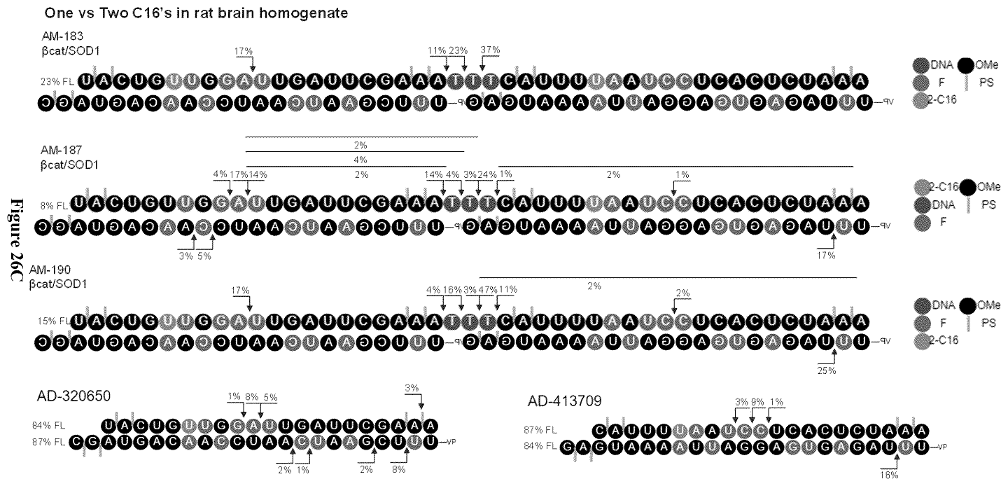

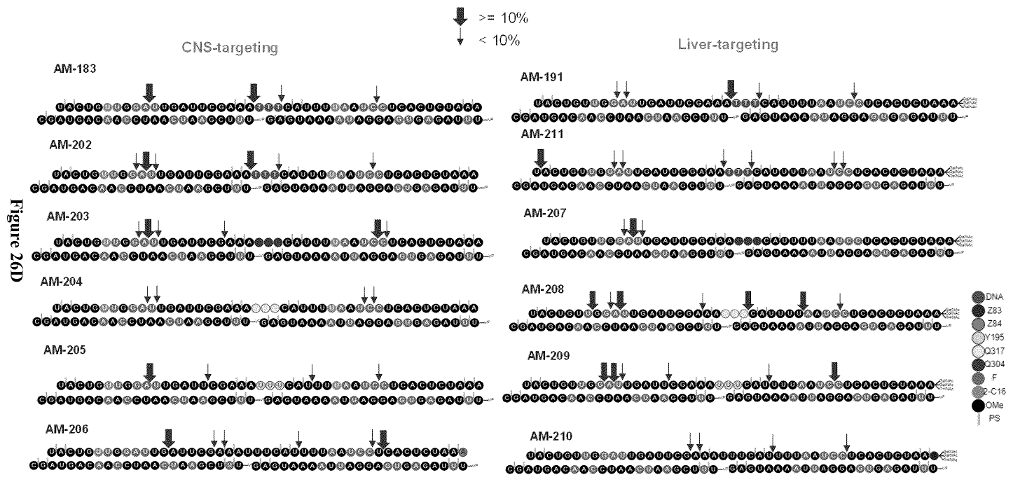

The in vitro stability of the different linker chemistries in the exemplary bis-siRNAs (AM-183 to AM-186) in rat brain homogenate were assessed and compared, and the results are summarized in FIG. 26A. The in vitro stability of the orietation chemistries in the exemplary bis-siRNAs (AM-183, AM-184, AM-188, and AM-189) in rat brain homogenate were assessed and compared, and the results are summarized in FIG. 26B. The in vitro stability of the exemplary bis-siRNAs possessing a single C₁₆ modification at SOD1-targeting siRNA or CTNNB1-targeting siRNA (AM-190 and AM-187) and the bis siRNA complex possessing a dual C₁₆ modification at both SOD1-targeting siRNA and CTNNB1-targeting siRNA (AM-183) in rat brain homogenate were assessed and compared, which was also compared against the mixture of duplexes (AD-320650 and AD-413709), and the results are summarized in FIG. 26C.

The metabolic liabilities of the exemplary bis-siRNAs in rat brain homogenate for CNS-targeting (AM-183, AM-202, AM-203, AM-204, AM-205, and AM-206) and for liver-targeting (AM-191, AM-207, AM-208, AM-209, AM-210, and AM-211) were assessed and compared, and the results are summarized in FIG. 26D.

### Example 12: Synthesis of Bis Small Interfering (Bis siRNAs) and Bis Small Circular Interfering RNA (Bis sciRNA) Conjugates, and In Vitro and In Vivo Gene Silencing

### Synthesis of Oligonucleotides

Oligonucleotides were synthesized on a Bioautomation Mermade 12 Synthesizer using commercially available RNA amidites: 5′-O-(4,4′-dimethoxytrityl)-2′-deoxy-2′-fluoro- and 5′-O-(4,4′-dimethoxytrityl)-2′-O-methyl-3′-O-(2-cyanoethyl-N,N-diisopropyl) phosphoramidite monomers of uridine, 4-N-acetylcytidine, 6-N-benzoyladenosine and 2-N-isobutyrylguanosine using standard solid-phase oligonucleotide synthesis protocols. The 2′-C16 ligand was covalently linked to the siRNA by a phosphodiester linkage. Phosphorothioate linkages were introduced by sulfurization of phosphite linkages utilizing 0.1 M 3-((N,N-dimethyl-aminomethylidene)amino)-3H-1, 2, 4-dithiazole-5-thione (DDTT) in pyridine.

After synthesis, the support was treated on column with 0.5 M piperidine in acetonitrile (ACN) for 15 minutes. The column was washed with ACN and then treated again with 0.5 M piperidine in ACN for an additional 15 minutes, then washed again with ACN. The support was dried under vacuum, added to a sealable container, and heated at 35° C. in aqueous ammonium hydroxide (28-30%) in ethanol (2:1) with shaking overnight. The oligonucleotide was filtered to remove the support with 5× volume of water and analyzed by LC-MS and ion-exchange HPLC.

After deprotection and crude quality confirmation, ion-exchange HPLC purification was performed. Purification buffer A consisted of 20 mM sodium phosphate, 15% ACN, pH 8.5. Buffer B was the same composition with an additional 1 M sodium bromide. TSKgel Super Q-5PW (20) anion exchange resin (Tosoh Corporation, 0018546) was used for purification, and a general purification gradient of 15% to 45% in about 20 column volumes was applied. Fractions were analyzed by ion-exchange analysis using a Dionex DNAPac PA200 ion-exchange analytical column, 4 mm×250 mm (ThermoFisher, 063000) at room temperature. Buffer A consisted of 20 mM sodium phosphate, 15% acetonitrile, pH 12. Buffer B was identical with additional 1 M sodium bromide. A gradient of 30% to 50% over 12 min with a flow rate of 1 mL/min was used to analyze fractions. Fractions with greater than 85% purity were pooled, concentrated, and desalted over size exclusion columns (GE Healthcare, 17-5087-01) with a flow rate of 10 mL/min.

Oligonucleotides were synthesized on a MerMade-12 DNA/RNA synthesizer. Sterling solvents/reagents from Glen Research, 500-A controlled pore glass (CPG) solid supports (Prime Synthesis), 2′-deoxy 3′-phosphoramidites (Thermo), and 2′-O-methyl (2′-OMe), 2′-deoxy-2′-fluoro (2′-F) ribonucleoside 3′-phosphoramidites (Hongene) were all used as received. The 2′-OMe-uridine-5′-bis-POM-(E) vinylphosphonate (VP) 3′-phosphoramidite (synthesized according to the procedures described in Parmar et al., “Facile synthesis, geometry, and 2′-substituent-dependent in vivo activity of 5′-(E)- and 5′-(Z)-vinylphosphonate-modified siRNA conjugates,” J. Med. Chem., 61: 734-44 (2018), which is incorporated herein by reference in its entirety) was dissolved to 0.15 M in 85% acetonitrile 15% dimethylformamide (DMF) and coupled using standard conditions on the synthesizer. GalNAc CPG support (L, Scheme I) was prepared and used as described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety. 5-Bromohexyl phosphoramidite (Glen Research, Cat #10-1946) was dissolved to 0.15 M in acetonitrile and coupled using standard conditions on the synthesizer. 3′-alkyne CPG support and 3′-alkyne hydroxyprolinol phosphoramidite (Y, Scheme I) was prepared and used as described in Jayaprakash et al., “Non-nucleoside building blocks for copper-assisted and copper-free click chemistry for the efficient synthesis of RNA conjugates,” Org. Lett., 12: 5410-13 (2010), which is incorporated herein by reference in its entirety. 2′-propargyl adenosine phosphoramidite was commercially available and use as received with universal Unylinker CPG support. Low-water content acetonitrile was purchased from EMD Chemicals. DNA and RNA oligonucleotides were synthesized using modified synthesis cycles, based on those provided with the instrument. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M I₂ in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase syntheses (SPS), the CPG solid support was washed with 5% (v/v) piperidine in anhydrous acetonitrile three times with 5-minute holds after each flow. The support was then washed with anhydrous acetonitrile and dried with argon. The oligonucleotides were then incubated with 28-30% (w/v) NH₄OH at 35° C. for 20 hours. For VP-containing oligonucleotides, after completion of the SPS, the CPG solid support was incubated with 28-30% (w/v) NH₄OH, where 5% of (v/v) of diethylamine was added at 35° C. for 20 hours (see O'Shea et al., “An efficient deprotection method for 5′-[0, O-bis(pivaloyloxymethyl)]-(E)-vinylphosphonate containing oligonucleotides,” Tetrahedron, 74: 6182-86 (2018), which is incorporated herein by reference in its entirety). The solvent was collected by filtration and the support was rinsed with water prior to analysis. Oligonucleotide solutions of ˜1 OD260 units/mL were used for analysis of the crudes, where 30-50 μL of solution were injected. LC/ESI-MS was performed on an Agilent 6130 single quadrupole LC/MS system using an XBridge C₈ column (2.1×50 mm, 2.5 μm) at 60° C. Buffer A consisted of 200 mM 1,1,1,3,3,3-hexafluoro-2-propanol (HFIP) and 16.3 mM triethylamine (TEA) in water, and buffer B was 100% methanol. A gradient from 0% to 40% of buffer B over 10 minutes followed by washing and recalibration at a flow rate of 0.70 mL/min. The column temperature was 75° C.

All oligonucleotides were purified and desalted, and further annealed to form GalNAc-siRNAs, based on the procedures described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety.

Circulation of Sense-Strand Oligonucleotides and Formation of sciRNA Conjugates—5′-Azido and “Click” Chemistry Synthesis for Circular Oligonucleotides.

To functionalize the sense strand, commercially available 5-bromohexyl phosphoramidite was coupled to the 5′ end during solid-phase synthesis. To provide the 6-azidohexyl click chemistry “handle”, 10 μmol of CPG loaded with 5′-(5-bromohexyl) modified oligonucleotides (see Lietard et al., “An efficient reagent for 5′-azido oligonucleotide synthesis,” Tetrahedron Lett., 48, 8795-98 (2007), which is incorporated herein by reference in its entirety) were suspended in 15 mL of an anhydrous DMF solution containing 130 mg of sodium azide and 300 mg of sodium iodide. The mixture was vigorously shaken at 65° C. for 75 minutes. After cooling down, the solution was filtered off and the CPG beads with the resulting 5′-(5-azidohexyl) solid-supported oligonucleotides were washed with DMF (2×10 mL) and dried under a stream of argon. The oligonucleotides were released from the solid support and purified and desalted as described above. The oligonucleotides were then dissolved in water to a concentration of ˜10 OD₂₆₀ units/mL. For a copper(I)-catalyzed alkyne-azide cycloaddition (CuAAC) “click” cyclization reaction yielding Z (Scheme I), 2 mL of the oligonucleotide solution (˜200 OD260 units) were mixed with 2 mL of methanol, 1.1 mL of sodium L-ascorbate (0.1 mM) and 1.1 mL of copper sulfate (20 mM). The reaction mixture was placed into a microwave (MW) tube container, equipped with a stirring bar, and placed in a MW reactor for 40 minutes at 60° C. (power ˜8 W, stirring, cooling, P=0). This protocol is a modification of that described in Lietard et al., “New strategies for cyclization and bicyclization of oligonucleotides by click chemistry assisted by microwaves,” J. Org. Chem., 73, 191-200 (2008), which is incorporated herein by reference in its entirety. All cyclic oligonucleotides were purified and desalted, and further annealed with an antisense strand to form the sciRNA and bis-sciRNAs, as described above.

### Enzymatic Stability Assays

3′ Exonuclease SVPD stability assay. Modified oligonucleotide was added at 0.1 mg/mL to a solution of 50 mM Tris-HCl (pH 7.2) and 10 mM MgCl₂. Snake Venom Phosphodiesterase (SVPD) (Worthington, Cat #LS003926) was added to the mixture at 750 mU/mL. Immediately after addition of the enzyme, the sample was injected onto a Dionex DNAPac PA200 column (4 mm×250 mm) at 30° C. and run at a flow rate of 1 mL/minute with a gradient of 40-55% Buffer B over 7.5 minutes. Buffer A was 20 mM sodium phosphate, 15% acetonitrile, pH 11; and Buffer B was Buffer A containing 1 M sodium bromide (pH 11). Aliquots were analyzed every hour for 24 hours. The area under the peak corresponding to full-length oligonucleotide was normalized to the area from the 0-hour time point (first injection). First order decay kinetics were assumed in calculation of half-lives. A control oligonucleotide, dT₁₉•dT (where dT is 2′-deoxythymidine, and • is a single 3′-terminal phosphorothioate linkage) was analyzed each day, and half-lives were reported relative to half-life of the control sequence. Enzyme was prepared as a stock of 1000 mU/mL aliquoted into 1 mL tubes and stored at −20° C. A new aliquot was used each week. Experiments were performed in triplicates.

5′ Exonuclease Phosphodiesterase II stability assay. Modified oligonucleotide was added at 0.1 mg/mL to a solution of 50 mM sodium acetate (pH 6.5) and 10 mM MgCl₂. Phosphodiesterase II from bovine spleen (Worthington, Cat #LS003 602) was added to the mixture at 500 mU/mL. Immediately after addition of the enzyme, the sample was injected onto a Dionex DNAPac PA200 column (4 mm×250 mm) at 30° C. and run at a flow rate of 1 mL/minute with a gradient of 37-52% Buffer B over 7.5 minutes. Buffer A was 20 mM sodium phosphate, 15% acetonitrile, pH 11; and Buffer B was Buffer A containing 1 M sodium bromide (pH 11). Aliquots were analyzed every hour for 24 hours. The area under the peak corresponding to full-length oligonucleotide was normalized to the area from the 0-hour time point (first injection). First order decay kinetics were assumed in calculation of half-lives. A control oligonucleotide, dT•dT19 (where dT is 2′-deoxythymidine, and • is a single 5′-terminal phosphorothioate linkage) was analyzed each day, and half-lives were reported relative to half-life of the control sequence. Enzyme was prepared as a stock of 2000 mU/mL, aliquoted into 1 mL tubes and stored at −20° C. A new aliquot was used each week. Experiments were performed in triplicates.

### Analysis of Oligonucleotide Stability in Plasma and Liver Homogenates

Rat plasma (BioIVT, Cat #RAT00PL38NCXNN) and liver homogenate (BioIVT, custom order) were diluted with a 10× cofactor solution to achieve a final concentration of 1 mM MgCl₂, 1 mM MnCl₂, and 2 mM CaCl₂). The sciRNA was added to 50 μL of the plasma or the liver homogenate to achieve the final concertation of 20 μg/mL. The reaction mixture was incubated with gently shaking at 37° C. At each predetermined time point (0, 1, 4, 8, and 24 hour), the reaction was stopped by adding 450 μL of Clarity OTX lysis-loading buffer (Phenomenex, Cat #AL0-8579) containing internal standard (oligonucleotide U21 at 1 μg/mL final concentration) and frozen at −80° C. until analysis. Experiments were performed in triplicate.

Oligonucleotide enrichment for LC-MS analysis was performed using Clarity OTX 96-well solid-phase extraction plates as described by Liu et al., “Oligonucleotide quantification and metabolite profiling by high-resolution and accurate mass spectrometry,” Bioanalysis, 11: 1967-80 (2019), which is incorporated herein by reference in its entirety. The SPE columns were conditioned initially with 1 mL of methanol followed by equilibration with 2 mL of 50 mM ammonium acetate with 2 mM sodium azide in HPLC-grade water. The samples were loaded on to SPE column by applying positive pressure. The columns were then washed five times with 1 mL of 50 mM ammonium acetate in 50/50 (v/v) water and acetonitrile (pH 5.5). Finally, the oligonucleotides are eluted using elution buffer containing 10 mM EDTA, 100 mM ammonium bicarbonate in 40/10/50 (v/v/v) acetonitrile/tetrahydrofuran/water (pH 8.8). The eluant was dried under nitrogen and resuspended in 120 μL of LC-MS grade water for LC-MS analysis.

Relative quantitation and metabolite identification of modified oligonucleotides was performed using high-resolution mass spectrometry on a Thermo Scientific Q Exactive coupled to ion-pairing reverse-phase liquid chromatography (Dionex Ultimate 3000) (LC-HRMS). A Waters X-Bridge BEH C8 XP Column (Cat #176002554, 130 Å, 2.5 μm, 2.1 mm×30 mm, 80° C.) was used for the chromatographic separation. The injection volume and flow rates were 30 μL and 1 mL/min, respectively. Mobile phase A consisted of 16 mM triethylamine (Sigma, Cat #471283), 200 mM 1,1,1,3,3,3-hexafluoro-2-propanol (Fisher, Cat #67-56-1) in LC-MS grade water (Fisher, Cat #7732-18-5); mobile phase B was 100% methanol (Fisher, Cat #67-56-1). The gradient started with 1% mobile phase B and progressed to 35% B over 4.3 minutes, then the column was equilibrated with 1% mobile phase B for 1 minute. The mass spectrometer data acquisition was performed in full scan mode with a scan range of 500-3000 m/z at a resolution setting of 35,000. Spray voltage was 2.8 kV. The auxiliary gas temperature and the capillary temperature were set to 300° C.

The Thermo Quan browser was used calculate the area ratio of extracted ion chromatograms (XIC) of test oligonucleotide to internal standard with 10 ppm mass accuracy. The m/z ions for used for XIC of the test oligonucleotide were 1018.7752, 1018.8868, 1019.1101, 1019.2220, 1019.3325, 1019.4436, 1019.5559, and 1019.6671, and m/z ions used for XIC of the internal standard were 1258.5073, 1258.8408, 1259.1740, 1259.8411, 1289.5061. After LC-HRMS analysis, data were processed using ProMass HR Deconvolution software (Novatia, LLC) to identify linearization and major metabolism of the modified oligonucleotides as described in Liu et al., “Oligonucleotide quantification and metabolite profiling by high-resolution and accurate mass spectrometry,” Bioanalysis, 11: 1967-80 (2019), which is incorporated herein by reference in its entirety.

The half-lives were calculated by monitoring loss of full-length test oligonucleotide for 24 hours, based on the method described in Chan et al., “Meeting the Challenge of Predicting Hepatic Clearance of Compounds Slowly Metabolized by Cytochrome P450 Using a Novel Hepatocyte Model, HepatoPac,” Drug Metab. Dispos. 41: 2024-32 (2013), which is incorporated herein by reference in its entirety. The amounts of test oligonucleotide and internal standard were normalized to time 0 hour for each time-point for respective oligonucleotides. The natural log of percentage full length remaining and the slope were calculated using linear regression. The half-life was calculated using equation (1):

$${t}_{\frac{1}{2}} = - \frac{Ln ( 2 )}{k} . \qquad ( 1 )$$

### Thermal Melting Studies

Melting studies were performed in 1 cm path length quartz cells on a Beckman DU800 spectrophotometer equipped with a thermoprogrammer. Duplexes were diluted to obtain a final concentration of ˜1 μM in 0.1×PBS buffer (pH 7.4) by adding 16 μL of stock solution of duplexes (1 mg/mL in 1.0×PBS) to 1 mL of 0.1×PBS buffer (pH 7.4). Melting curves were monitored at 260 nm with a heating rate of 1° C./minute from 10 to 90° C. Melting temperatures (Tₘ) were calculated from the first derivatives of the heating curves (in built software) and the reported values are the result of two independent measurements.

### NMR Studies

Lyophilized RNA was dissolved in a mixture of 10% ²H₂O/90% H₂O with 20 mM NaCl and 10 mM sodium phospate buffer (pH 7). Equimolar ratios of sense and antisense strands were mixed to constitute linear or circular duplexes. Final concentrations of duplexes in 600 μL were in the range from 20 to 60 μM. All spectra were acquired at 25° C. on an Agilent VNMRS 800 MHz NMR spectrometer equipped with a cold probe.

### In Vitro Silencing Activity Studies

Transfection. Primary Mouse Hepatocytes (PMH) were transfected with siRNA to test for silencing efficiency. siRNA (5 μL) at the indicated concentrations was mixed with 4.9 μL of Opti-MEM and 0.1 μL of Lipofectamine RNAiMax (Invitrogen, Cat #13778-150) per well of a 384-well plate and incubated at room temperature. After 15 minutes, 40 μL of William's E medium or EMEM medium containing approximately 5×10³ cells were added to the wells. Cells were incubated for 24 hours prior to RNA purification.

Free uptake. Free uptake experiments were carried out in PMH. siRNA (5 μL at the indicated concentration) was mixed with 5 μL of Opti-MEM per well of a 384-well plate. After 15 min, 45 μL of William's E medium or EMEM medium containing approximately 5×10³ cells were added to the wells. Cells were incubated for 48 hours prior to RNA purification.

Total RNA isolation using Dynabeads mRNA isolation kit. RNA was isolated using an automated protocol on a BioTek-EL406 platform using Dynabeads (Invitrogen, Cat #61012). 50 μL of lysis/binding buffer (Tris HCl pH 7.5, LiCl, EDTA pH 8.0, DTT) and 25 μL of lysis/binding buffer containing 3 μL of magnetic beads were added to each well. The plates were incubated on an electromagnetic shaker for 10 minutes at room temperature, then the magnetic beads were captured, and the supernatant was removed. The bead-bound RNA was washed twice with 150 μL/well of Buffer A (Tris HCl pH 7.5, LiCl, EDTA pH 8.0, DTT), and then washed once with 150 μL/well of Buffer B (Tris HCl pH 7.5, LiCl, EDTA pH 8.0). The beads were then washed with 150 μL of Elution Buffer and re-captured, and the supernatant was collected.

cDNA synthesis using ABI High-capacity cDNA reverse transcription kit. cDNA synthesis was performed using an ABI kit (Cat #4368813). To the wells of a 384-well plate containing the RNA isolated using Dynabeads was added 10 μL of a master mix containing 1 μL 10×Buffer, 0.4 μL 25×dNTPs, 1 μL 10× random primers, 0.5 μL reverse transcriptase, 0.5 μL RNase inhibitor and 6.4 μL of nuclease free water. The plates were sealed, mixed, and incubated on an electromagnetic shaker for 10 minutes at room temperature, followed by 2 hours at 37° C.

Gene expression analysis (RT-qPCR). All probes for RNA quantification were acquired from Life Technologies utilizing their Taqman gene expression system with dual labeled probes which allowed for analysis of gene expression. Target gene expression was normalized to the Gapdh ubiquitous control in each well utilizing a dual label system. Ct values were measured using a Light Cycler 480 (Roche). To calculate relative fold change, real time data were analyzed using the ΔΔCt method and normalized to assays performed with cells treated with a non-targeting siRNA control. Taqman probe catalogue numbers: Mouse C5 (Mm00439275_m1), Mouse Gapdh 4352339E, Mouse Ttr (Mm00443267_m1).

### In Vivo Studies of Silencing Activity in Mice

All procedures using mice were conducted by certified laboratory personnel using protocols consistent with local, state, and federal regulations. Experimental protocols were approved by the Institutional Animal Care and Use Committee (IACUC), the Association for Assessment and Accreditation of Laboratory Animal Care International (accreditation number: 001345), and the office of Laboratory Animal Welfare (accreditation number: A4517-01). The number of samples for animal studies was determined to be one that would allow for confidence in the resulting data set utilizing the least number of animals, as required in accordance with IACUC guidelines. All animals were acclimated in-house for 48 hours prior to study start. Female C57BL/6 mice approximately 8 weeks of age were obtained from Charles River Laboratories and randomly assigned to each group. All animals were treated in accordance with IACUC protocols.

Animals were dosed subcutaneously at 10 μL/g with sciRNA, siRNA duplex, or with PBS saline control. Doses used in this study were 3 mg/kg. The sciRNAs and siRNAs were diluted into phosphate buffered saline (PBS, pH 7.4). All dosing solutions were stored at 4° C. until time of injection. Animals were sacrificed at either 5 or 7 days post dose. Livers were harvested and snap frozen for analysis.

Serum collection. Blood was collected utilizing the retro-orbital eye bleed procedure 24 h post the final dose in accordance with the IACUC approved protocol. The sample was collected in Becton Dickinson serum separator tubes (Fisher Scientific, Cat #BD365967). For analysis of TTR, serum samples were kept at room temperature for 1 hour and then spun in a micro-centrifuge at 21,000×g at room temperature for 10 minutes. Serum was transferred into 1.5 mL micro-centrifuge tubes for storage at −80° C. until the time of assay. Serum collected for the analysis of circulating C5 was kept at room temperature for 15 minutes, and then immediately transferred to 4° C. prior to spinning in a micro-centrifuge at 21,000×g at room temperature for 10 minutes. Serum was transferred into 1.5 mL micro-centrifuge tubes for storage at −80° C. until the time of assay.

Circulating serum transthyretin (TTR) levels. Serum samples were diluted 1:4,000 and assayed using a commercially available kit from ALPCO specific for detection of mouse prealbumin (Cat #41-PALMS-E01). Protein concentrations (μg/mL) were determined by comparison to a purified TTR standard, following the manufacturer's instructions.

Circulating serum complement 5 (C5) levels. An ELISA assay was developed to specifically detect circulating mouse C5 levels. The primary antibody was goat-anti-human C5 (Complement Technologies, Cat #A220), and the secondary antibody was bovine anti-goat IgG-HRP (Jackson ImmunoResearch, Cat #805-035-180), which had minimal cross-reactivity to other species. Antibodies were used at 0.8 mg/mL. The assay was developed using a TMB substrate kit (R&D Systems, Cat #DY999), and the reaction was stopped using sulfuric acid prior to measurement. The serum samples were diluted 1:5,000 for analysis.

### In Vivo Liver Exposure and Ago2 Loading

Mice were sacrificed on day 7 post-dose. Livers were snap frozen in liquid nitrogen and ground into powder for further analysis. Total siRNA liver levels were measured by reconstituting liver powder at 10 mg/mL in PBS containing 0.25% Triton-X 100. The tissue suspension was further ground with 5-mm steel grinding balls at 50 cycles/s for 5 minutes in a tissue homogenizer (Qiagen TissueLyser LT) at 4° C. Homogenized samples were then heated at 95° C. for 5 minutes, briefly vortexed, and allowed to rest on ice for 5 minutes. Samples were then centrifuged at 21,000×g for 15 minutes at 4° C. The siRNA-containing supernatants were transferred to new tubes. The siRNA sense and antisense strand levels were quantified by stem loop reverse transcription followed by Taqman PCR (SL-RT QPCR)(see the methods described in Chen et al., “Real-time quantification of microRNAs by stem-loop RT-PCR,” Nucleic Acids Res., 33: e179-e179 (2005); and Pei et al., “Quantitative evaluation of siRNA delivery in vivo,” RNA, 16: 2553-2563 (2010), which are incorporated by reference in their entirety) and adapted to chemically modified siRNAs.

Ago2-bound siRNA from mouse liver was quantified by preparing liver powder lysates at 100 mg/mL in lysis buffer (50 mM Tris-HCl, pH 7.5, 150 mM NaCl, 2 mM EDTA, 0.5% Triton-X 100) supplemented with freshly added protease inhibitors (Sigma-Aldrich, Cat #P8340) at 1:100 dilution and 1 mM PMSF (Sigma-Aldrich, Cat #P7626). Total liver lysate (10 mg) was used for each Ago2 immunoprecipitation (IP) and control IP. The siRNA not bound to Ago2 was cleaned up with pre-saturated QAE Resin (GE healthcare, Cat #17-0200-01) in lysis buffer (16 mg/ml) supplemented with protease inhibitors at 1:100 dilution and 1 mM PMSF. Samples were filtered through cellulose acetate filter (Fisher, Cat #P169702) to remove the resin before proceeding with the Ago2 IP. Anti-Ago2 antibody was purchased from Wako Chemicals (Clone No.: 2D4). Control mouse IgG was from Santa Cruz Biotechnology (Cat #sc-2025). Protein G Dynabeads (Life Technologies, Cat #10003D) were used to precipitate antibodies. Ago2-associated siRNAs were eluted by heating (50 μL PBS, 0.25% Triton X-100; at 95° C. for 5 minutes) and quantified by SL-RT QPCR (see the methods described in Chen et al., “Real-time quantification of microRNAs by stem-loop RT-PCR,” Nucleic Acids Res., 33: e179-e179 (2005); and Pei et al., “Quantitative evaluation of siRNA delivery in vivo,” RNA, 16: 2553-2563 (2010), which are incorporated by reference in their entirety).

### Molecular Modeling

Coordinates of the complex between human Ago2 and an antisense (guide, AS): passenger (sense, S) strand duplex with seed region pairing were retrieved from the Protein Data Bank (www.rcsb.org; ID code 4W5T) (36). In the complex, the S strand is comprised of residues S1 to S9 (5′ to 3′ direction). The Z linker was built with the program UCSF Chimera (37) using the structure-editing ‘build structure’ and ‘modify structure’ tools and starting from the 3′-terminus of the sense strand (S9). After adding a 3′-phosphate group, the linker was constructed using ideal bond lengths and angles and an extended conformation in the direction of the 5′-end of the S strand wherever possible, while avoiding short contacts with AS strand and Ago2 side chains of the Ago2 PIWI and MID domains and L2 linker region. The 5′-terminal S1 residue as observed in the crystal structure of the complex was looped around in order to get its 5′-hydroxyl group to point in the direction of the growing Z linker. After addition of a phosphate to the last methylene carbon of the linker, a distance of some 5 Å remained between 5′-OH of S1 and phosphorous. This distance was systematically shortened by altering S1 and linker torsion angles until a final distance of 1.6 Å allowed cyclization of the sense strand with acceptable bond and torsion angles.

### Chemical Synthesis of GalNAc-sciRNA Conjugates.

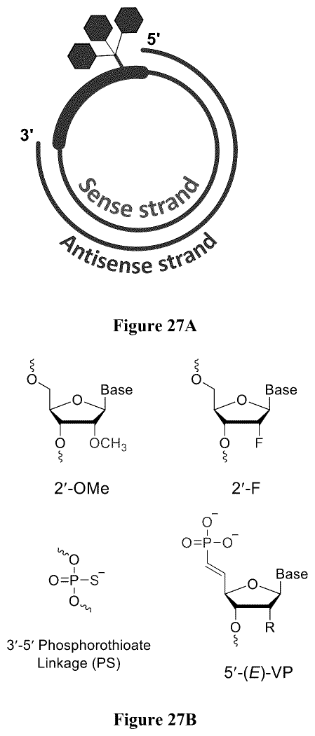

In this example, sciRNA constructs have been designed where the sense strand was cyclized and was conjugated at the 3′-end to a trivalent GalNAc ligand. As shown in FIG. 27A, the cyclized sense strand was then annealed to a linear antisense strand. Both sense and antisense strands were constructed with 2′-OMe, 2′-F, and PS backbone modifications, as shown in FIG. 27B.

The cyclization procedure is exemplified in Scheme I. As shown in Scheme I, an additional “click” chemistry “handle,” an N-alkyne linker as a hydroxyprolinol building block (Y) attached directly to the GalNAc ligand (L) solid support (see Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated by reference in its entirety), was introduced at the 3′-end of the sense strand (see Jayaprakash et al., “Non-nucleoside building blocks for copper-assisted and copper-free click chemistry for the efficient synthesis of RNA conjugates,” Org. Lett., 12: 5410-13 (2010), which is incorporated by reference in its entirety), via its phosphoramidite, in a standard solid-phase synthesis (SPS) setup. Both the hydroxyprolinol linker and the trivalent GalNAc ligand are clinically validated moieties in RNAi therapeutics.

After completion of the SPS of the entire sense stand sequence, the commercially available 6-bromohexyl phosphoramidite was coupled to the 5′-end of the sense strand. Subsequently, the bromine (1, Scheme I) was substituted with an azido group (2, Scheme I), providing the 6-azidohexyl handle (Q) (see Lietard et al., “An efficient reagent for 5′-azido oligonucleotide synthesis,” Tetrahedron Lett., 48: 8795-98 (2007), which is incorporated by reference in its entirety). After cleavage from the solid support, deprotection and purification of the full-length 5′-azido oligonucleotide, the copper(I)-catalyzed alkyne-azide cycloaddition (CuAAC) “click” reaction allowed for the completion of cyclized, circular sense strands of the sciRNA (see Lietard et al., “New strategies for cyclization and bicyclization of oligonucleotides by click chemistry assisted by microwaves,” J. Org. Chem., 73: 191-200 (2008); and Huisgen, “Kinetics and reaction mechanisms: selected examples from the experience of forty years,” Pure and Appl. Chem., 61: 613-28 (1989), both of which are incorporated by reference in their entirety). Annealing with the corresponding complementary antisense strands yielded the target sciRNA duplexes.

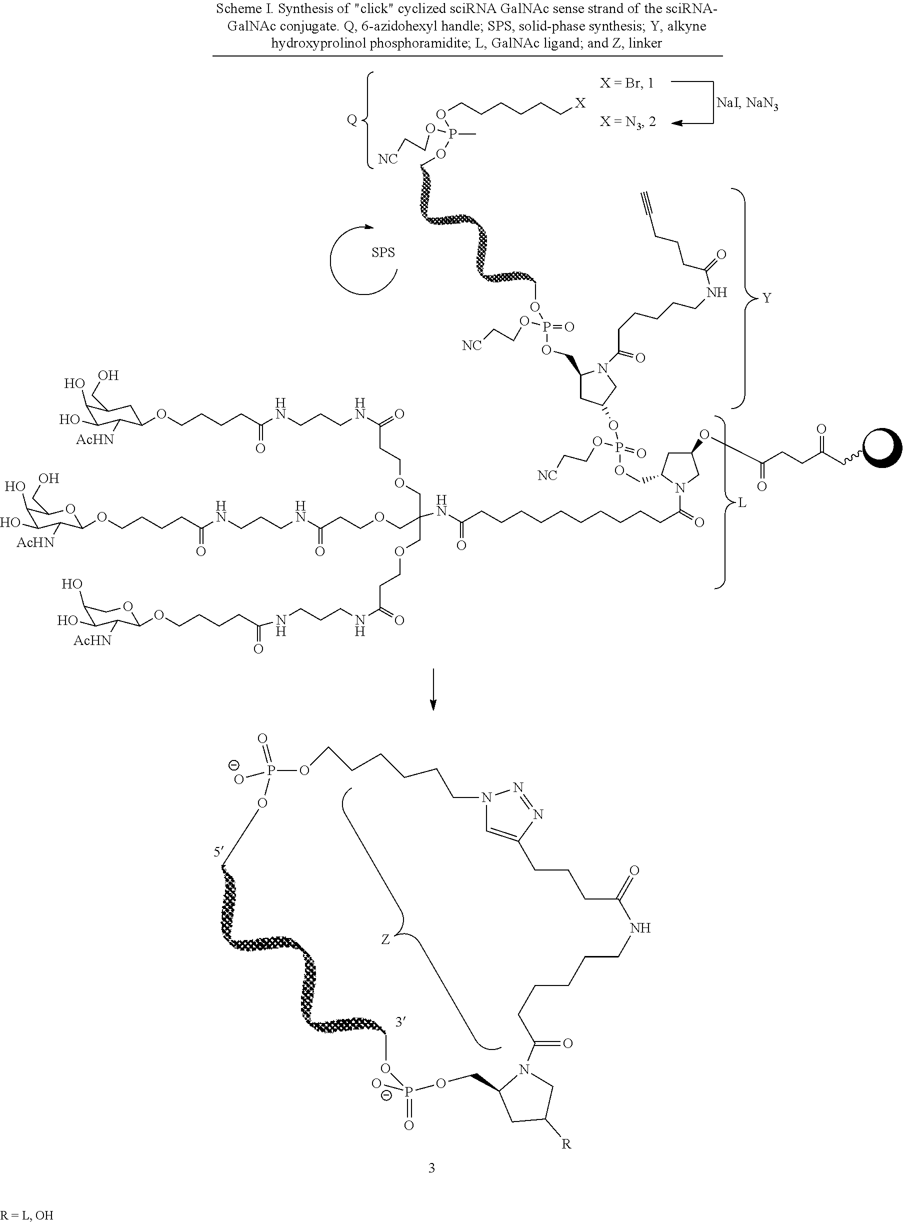

The oligonucleotides used in this example are listed and characterized in Table 6.

TABLE 6
Oligonucleotide sequences and mass spectra-based characterization

| Oligo- | | | Sense/ | Calc | Found |
|---|---|---|---|---|---|
| nucleotide | Sequences (5′-3′)^{a} | Duplex | Antisense | MW | MW |
| ON-1 | (dT)₁₉•dT | | | 6037.96 | 6036.95 |
| ON-2 | dT•(dT)₁₉ | | | 6037.96 | 6037.11 |
| ON-3 | Q(dT)₂₃Y | | | 7526.02 | 7524.37 |
| ON-4 | Z(dT)₂₃ | | | 7526.02 | 7524.47 |
| ON-5 | QaaAaCaGuGuUCUuGcUcUaUaAY | | | 8047.08 | 8045.22 |
| ON-6 | ZaaAaCaGuGuUCUuGcUcUaUaA | | | 8047.08 | 8044.98 |
| ON-7 | A•a•CaGuGuUCUuGcUcUaUaAL | si-1 | Sense | 8590.17 | 8588.69 |
| ON-8 | u•U•aUaGaGcAagaAcAcUgUu•u•u | si-1/si-3/ | Antisense | 7595.94 | 7594.79 |
| | | si-4 | | | |
| ON-9 | VPu•U•aUaGaGcAagaAcAcUgUu•u•u | si-2/si-5 | Antisense | 7672.94 | 7670.55 |
| ON-10 | QA•a•CaGuGuUCUuGcUcUaUaAYL | si-3 | Sense | 9181.71 | 9179.98 |
| ON-11 | ZA•a•CaGuGuUCUuGcUcUaUaAL | si-4/si-5 | Sense | 9181.71 | 9179.71 |
| ON-12 | G•a•CaAaAuAACuCaCuAuAaUL | si-6 | Sense | 8627.29 | 8626.45 |
| ON-13 | a•U•uAuAgUgAguuAuUuUgUc•a•a | si-6/si-7 | Antisense | 7574.87 | 7573.88 |
| ON-14 | ZG•a•CaAaAuAACuCaCuAuAaUL | si-7 | Antisense | 9218.83 | 9217.13 |

^{a}Chemistry modifications are indicated as follows: •, PS linkage; lower case nucleotides, 2′-OMe; upper case nucleotides in italics, 2′-F; Q, 6-azidohexyl-phosphate; VP, 5′-(E)-vinylphosphonate. For structures of L, Y, and Z, see Scheme I. Z denotes a cyclic oligonucleotide.

### Enzymatic Stability of Cyclic Oligonucleotides.

To assess the stabilization that the cyclic modification Z can impart to the oligonucleotide nuclease resistance, in vitro assay using either a 3′-exonuclease or 5′-exonuclease, paired with their appropriate assay controls, were utilized. The results of the enzymatic stability of linear and circular single-stranded oligonucleotides, compared against the controls (ON-1 and ON-2) are shown in Table 7.

TABLE 7
Enzymatic stability of linear and circular single-stranded oligonucleotides.

| | | 3′- | Intact | 5′- | Intact |
|---|---|---|---|---|---|
| Oligo- | | Exonuclease | after 24 | Exonuclease | after 24 |
| nucleotide | Sequences (5′-3′)ª | half-life^{b} | h (%) | half-life^{c} | h (%) |
| ON-1/ON-2 | (dT)₁₉•dT/dT·(dT)₁₉ | 1 | 10.0 | 1 | 6.5 |
| ON-3 | Q(dT)₂₃Y | 0.7 | 2.7 | 8.2 | 13.3 |
| ON-4 | Z(dT)₂₃ | 1.2 | 14.8 | 26.8 | 52.5 |
| ON-5 | QaaAaCaGuGuUCUuGcUcUaUaAY | 12.8 | 76.1 | 14.1 | 83.8 |
| ON-6 | ZaaAaCaGuGuUCUuGcUcUaUaA | 14.5 | 79.1 | 423.0 | 100.0 |

^{a}Chemistry modifications legend: •-phosphorothioate (PS) linkage; dT-2′-deoxythimidine; lower case nucleotides-2′-O-methyl (OMe); upper case nucleotides in italics-2′-deoxy-2′-fluoro (F); Q = 6-azidohexyl-phosphate; structures of Y and Z-see Scheme I; and Z denotes a cyclic oligonucleotide.
^{b}Half-life value determined in hours and reported relative to that of the (dT)₁₉•dT control.
^{c}Half-life value was determined in hours and reported relative to that of the dT•(dT)₁₉ control. First order decay kinetics were assumed in calculation of half-lives. The data is representative of two independent experiments with similar results.

As shown in Table 7, single-stranded 23-mer phosphodiester oligonucleotides were composed either of full 2′-deoxythymidine (ON-3 and ON-4) or of fully chemically 2′-modified nucleotides (ON-5 and ON-6). Each series included the circular oligonucleotide strands containing the “clicked” Z chemical modification (ON-4 and ON-6), as well as the linear oligonucleotides containing the “unclicked” precursor moieties Q and Y (ON-3 and ON-5).

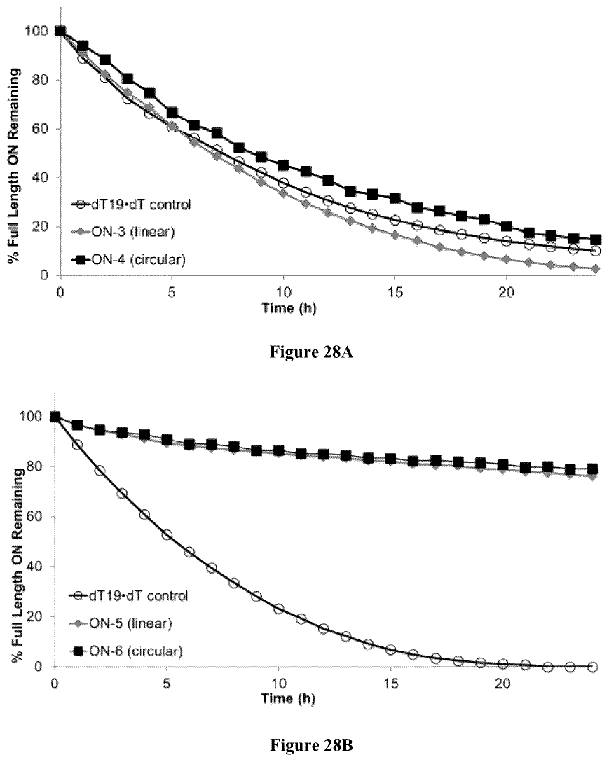

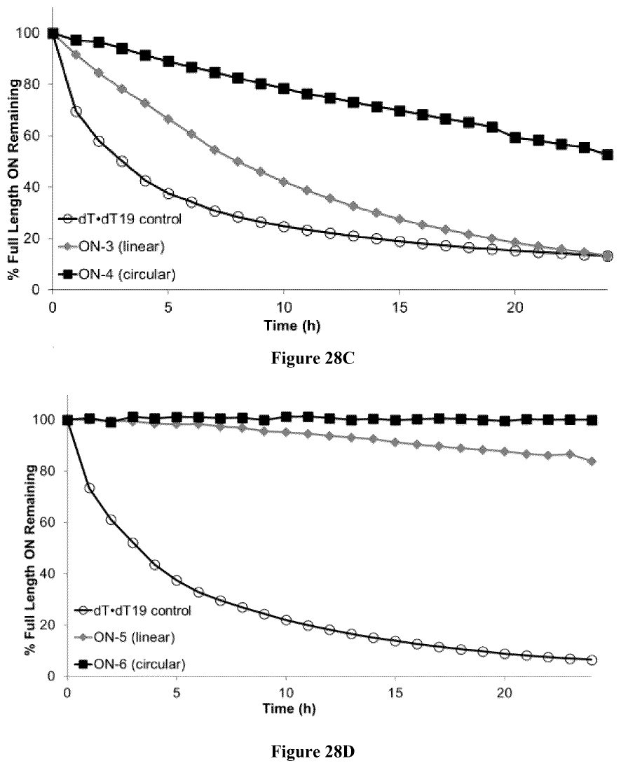

As shown in FIGS. 28A-28D, the results from the exonuclease assays revealed that the cyclic modification Z provided higher exonuclease stability than the assay controls as well as the linear counterparts. ON-4 (deoxythymidine) and ON-6 (chemically modified), were more stable than the linear counterparts ON-3 and ON-5, respectively) and than the control oligonucleotides (ON-1 and ON-2). The stabilization was even further enhanced by the introduction of the fully chemically 2′-modified nucleotides in the modified series, where the cyclized, fully chemically 2′-modified oligonucleotide ON-6 was virtually stable to exonuclease action, i.e., not degraded to a significant extent by either nuclease over the time course of the experiment (FIG. 28D). In the unmodified 2′-deoxy series (ON-3 and ON-4), the cyclic modification Z provided higher stabilization towards 5′-exonuclease (FIG. 28C) than towards 3′-exonuclease (FIG. 28A). Likewise, the 3′-exonuclease assays showed less differentiation between linear and circular oligonucleotides than the 5′-exonuclease (FIGS. 28A and 28C).

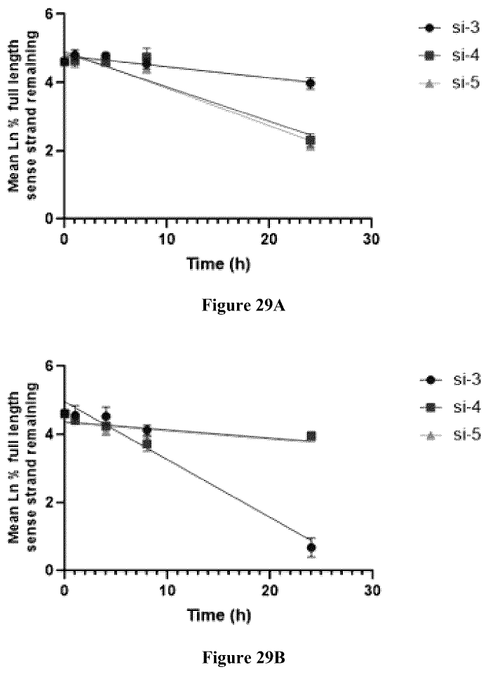

To study the metabolism of the circular siRNA designs, the stability studies were extended from the individual single strands to the annealed sciRNA duplexes. The in vitro stabilities of two sciRNAs (si-4 and si-5, see Table 9 below) and the linear siRNA containing the same nucleotide sequence and modifications (si-3) were assessed in rat plasma and rat liver homogenates. The results are shown in FIGS. 29A-29B. Metabolite identifications of si-3, si-4, and si-5 based on the LC-MS analysis from rat liver homogenate or rat plasma were summarized in Table 8A, Table 8B, and Table 8C, respectively.

As shown in FIGS. 29A-29B, no degradation of either the circular sciRNA strands or the linear siRNA strands was detected in plasma for up to 8 hours (FIG. 29A). However, in metabolite analysis by LC-MS, after 24 hours of incubation in rat plasma, an addition of a water molecule to the circular strand was observed (Tables 8B and 8C). This may have resulted from opening of the cyclic structure, yielding in linearized oligonucleotide. In rat liver homogenates, the circular sciRNAs (si-4 and si-5) were more stable than the linear counterpart (si-3). There was no degradation of si-4 or si-5 at 24 hours, whereas for si-3, only ˜55% of the full-length sense strand was detected at 24 hour (FIG. 29B). The sciRNAs have significantly longer half-lives of 29 and 30 hours compared to the linear siRNA, which has a 4-hour half-life. This correlates with the results of the analysis of the stabilities of the single strands, where circularization enhanced stability in the presence of exonucleases. In the liver homogenates, opening of the cyclic structure of the sense strands of either si-4 or si-5 was not observed (Tables 8B and 8C).

TABLE 8A
Metabolite identification of si-3 from rat liver homogenate or plasma

| Observed | | | |
|---|---|---|---|
| mass | Possible metabolite sequence | Strand | Description^{a} |
| 7592.52 | U*U*aUaGaGcAagaAcAcUgUu*u*u | Antisense | Parent |
| 6588.965 | UaGaGcAagaAcAcUgUu*u*u | Antisense | 5′ n-4 |
| 6371.919 | Pu*U*aUaGaGcAagaAcAcUg | Antisense | 3′ n-4 |
| 6291.957 | U*U*aUaGaGcAagaAcAcUg | Antisense | 3′ n-4 |
| 5700.846 | VPu*U*aUaGaGcAagaAcAc | Antisense | 3′ n-5 |
| 9177.039 | QA*a*CaGuGuUCUuGcUcUaUaAL | Sense | Parent |
| 8973.962 | ZA*a*CaGuGuUCUuGcUcUaUaAL | Sense | -GalNAc₁ |
| | (-GalNAc₁) | | |

^{a}-GalNAc₁ refers to loss of single GalNAc sugar from the triantenary ligand.

TABLE 8B
Metabolite identification of si-4 from rat liver homogenate or plasma

| Observed | | | |
|---|---|---|---|
| mass | Proposed metabolite sequence | Strand | Description^{a} |
| 7592.52 | U*U*aUaGaGcAagaAcAcUgUu*u*u | Antisense | Parent |
| 6588.965 | UaGaGcAagaAcAcUgUu*u*u | Antisense | 5′ n-4 |
| 6291.957 | U*U*aUaGaGcAagaAcAcUg | Antisense | 3′ n-4 |
| 9177.039 | QA*a*CaGuGuUCUuGcUcUaUaAYL | Sense | Parent |
| 9195.049 | QA*a*CaGuGuUCUuGcUcUaUaAYL | Sense | Hydrolyzed |

^{a}Hydrolyzed = parent strand +18.015 Da.

TABLE 8C
Metabolite identification of si-5 from rat liver homogenate or plasma

| Observed | | | |
|---|---|---|---|
| mass | Proposed metabolite sequence | Strand | Description^{a} |
| 7668.030 | VPU*U*aUaGaGcAagaAcAcUgUu*u*u | Antisense | Parent |
| 6588.965 | VPu*U*aUaGaGcAagaAcAcUgP | Antisense | 5′ n-4 |
| 6447.9166 | Pu*U*aUaGaGcAagaAcAcUg | Antisense | 3′ n-3 |
| 5700.846 | QA*a*CaGuGuUCUuGcUcUaUaAYL/ | Antisense | 3′ n-5 |
| | ZA•a•CaGuGuUCUuGcUcUaUaAL | | |
| 9177.039 | QA*a*CaGuGuUCUuGcUcUaUaAYL | Sense | Parent |
| 9195.049 | Z49A*a*CaGuGuUCUuGcUcUaUaAL | Sense | Hydrolyzed |
| 8973.962 | Z49A*a*CaGuGuUCUuGcUcUaUaAL | Sense | -GalNAc₁ |
| | (-GalNAc₁) | | |

^{a}Hydrolyzed = parent strand +18.015 Da.

-GalNAc1 refers to loss of single GalNAc sugar from triantenary ligand.
Z49—cyclization by clicking 3′-phosphate-Hyp-C9-1,4-triazole-C6-5′-phosphate:

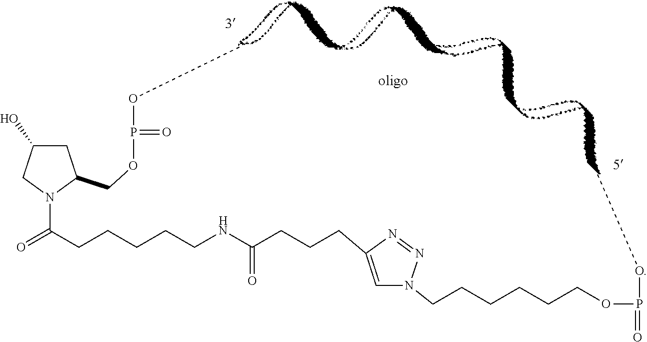

### Thermal Stability of GalNAc-sciRNA Conjugates and NMR Studies.

Table 9 shows the thermal stability of the circular GalNAc-sciRNA conjugates and linear GalNAc-siRNA conjugates. The cyclic 21-nt sense strands (siRNAs sense strands containing the Z modification) were hybridized with their respective complementary 23-nt antisense strands in order to prepare the sciRNA duplexes, and the melting temperature (Tₘ) of each complex was determined. For comparison, the Tₘ of the open (i.e., not circular) siRNA duplexes were also determined. These included the standard GalNAc-siRNA constructs formed from linear sense strands (si-1, si-2 and si-6), but also one representative siRNA fromed from linear sense strand (si-3), where the 5′ and 3′ sense strand modifications Q and Y, have not been engaged in the “click” reaction yielding the cyclic triazole Z moiety connecting the 5′ and 3′ ends of the siRNA sense strand.

As shown in Table 9, the sciRNA constructs (si-4, si-5 and si-7) showed an increased destabilization (by 15-20° C.) compared to the open counterpart (si-3) or standard siRNA duplexes. This thermal destabilization points to a strain of the cyclic 21-nt sense strand, which can impart flexibility in the siRNA duplex structure, and a subset of the nucleobases form Watson-Crick base pairs.

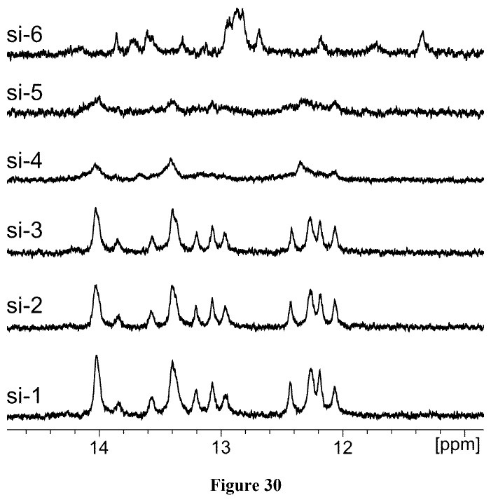

The solution-state ¹H NMR analysis of the siRNA duplexes in FIG. 30 show that the spectra of linear siRNA duplexes (Table 9, si-1, si-2, si-3 and si-6) exhibit well-resolved resonances. Several signals for imino proton could be observed in the region from δ 12 to 14 ppm, which is indicative of Watson-Crick base pairing. The number of signals suggests fully complementary duplexes are formed. The spectra of circular sciRNA duplexes (Table 9, si-4 and si-5) exhibit broad signals. But a few signals were observed in the imino regions, indicating a partial duplex structure, suggesting some thermal destabilization and a subset of the nucleobases form Watson-Crick base pairs.

TABLE 9
Thermal stability and in vitro potency in primary mouse hepatocytes (PMHs) of linear
GalNAc-siRNA and circular GalNAc-sciRNA conjugates.

| | | | Tm (° C.)^{b} | Transfection | Free uptake |
|---|---|---|---|---|---|
| siRNA | Target | Sequences (5′-3′)ª | (ΔTm)^{c} | IC₅₀ (pM)^{d} | IC₅₀ (pM)^{c} |
| si-1 | Ttr | A•a•CaGuGuUCUuGcUcUaUaAL | 68.2 | 151.1 ± 21.8 | 53.1 ± 15.6 |
| | | u•U•aUaGaGcAagaAcAcUgUu•u•u | | | |
| si-2 | Ttr | A•a•CaGuGuUCUuGcUcUaUaAL | 67.8 | 41.9 ± 13.8 | 21.6 ± 3.3 |
| | | VPu•U•aUaGaGcAagaAcAcUgUu•u•u | (−0.4) | | |
| si-3 | Ttr | QA•a•CaGuGuUCUuGcUcUaUaAYL | 67.3 | 81.3 ± 55.5 | 58.5 ± 1.9 |
| | | u•U•aUaGaGcAagaAcAcUgUu•u•u | (−0.9) | | |
| si-4 | Ttr | ZA•a•CaGuGuUCUuGcUcUaUaAL | 52.4 | 836.8 ± 140.4 | 197.9 ± 31.2 |
| | | u•U•aUaGaGcAagaAcAcUgUu•u•u | (−15.7) | | |
| si-5 | Ttr | ZA•a•CaGuGuUCUuGcUcUaUaAL | 52.5 | 124.7 ± 64.1 | 63.9 ± 9.1 |
| | | VPu•U•aUaGaGcAagaAcAcUgUu•u•u | (−15.7) | | |
| si-6 | C5 | G•a•CaAaAuAACuCaCuAuAaUL | 62.3 | 60.5 ± 30.1 | 109.2 ± 53.8 |
| | | a•U•uAuAgUgAguuAuUuUgUc•a•a | | | |
| si-7 | C5 | ZG•a•CaAaAuAACuCaCuAuAaUL | 41.8 | 830.2 ± 195.0 | 824.4 ± 72.7 |
| | | a•U•uAuAgUgAguuAuUuUgUc•a•a | (−20.5) | | |

^{a}Sense strand sequences are the top rows; antisense strand sequences are the bottom rows. Chemistry modifications legend: •-phosphorothioate (PS) linkage; lower case nucleotides-2′-O-methyl (OMe); upper case nucleotides in italics-2′-deoxy-2′-fluoro (F); sense strands (top rows); antisense strands (bottom rows); Q-6-azidohexyl-phosphate; structures of L, Y and Z-see Scheme I; VP refers to 5′-(E)-vinylphosphonate; and Z denotes a cyclic oligonucleotide.
^{b}Tₘ refers to the melting temperature obtained from the maxima of the first derivatives of the melting curves (A₂₆₀ vs temperature) recorded in 0.1 × PBS buffer (pH 7.4) using 1.0 μM concentrations of each strand.
c^{Δ}Tₘ refers to the change in melting temperature compared to the unmodified duplex (for si-2 through si-5, reference duplex is si-1, and for entry si-7, reference duplex is si-6). Average of two independent annealing sample preparations and measures (n = 2).
^{d}Compounds were added to PMHs by transfection with Lipofectamine RNAiMAX and after a 24 h incubation, cells were lysed and processed for RNA isolation, cDNA synthesis, and quantitative PCR analysis. mRNA levels were normalized relative to mouse Gapdh. Mean IC₅₀ values reported with SD (n = 4).
^{e}Compounds were added to PMHs by free uptake and after a 48 h incubation, cells were lysed and processed for RNA isolation, cDNA synthesis, and quantitative PCR analysis.

mRNA levels were normalized relative to mouse Gapdh. Mean IC₅₀ values reported with SD (n=4).

### In Vitro and In Vivo Activity of GalNAc-sciRNA Conjugates.

Several sciRNA designs were evaluated and potent gene expression silencing in vitro and in vivo were observed, albeit in some cases lower than that of the non-circular GalNAc-siRNA parents.

The in vitro potency of the sciRNAs was determined in primary mouse hepatocytes (PMHs), after either transfection of the siRNAs using Lipofectamine RNAi Max, or after simple free uptake within the PMHs via the GalNAc ligand and its uptake receptor, the asialoglycoprotein receptor (ASGPR), which is ubiquitously expressed on the surface of hepatocytes. The results of in vitro potency of linear GalNAc-siRNA and circular GalNAc-sciRNA conjugates in PMHs are shown in Table 9. Cyclic sciRNA constructs were prepared targeting two distinct targets, namely the mRNAs of rodent transthyretin (Ttr) or complement 5 (C5), alongside the standard GalNAc-siRNAs and the corresponding linear “unclicked” siRNA controls, having the Q and Y chemical modifications resulting in the cyclic “click” construct Z. Table 9 shows that using each of the two modes of cellular delivery, strong inhibition of target mRNA expression were observed in all cases, albeit with a loss of potency in the in vitro inhibition of mRNA levels with the cyclic sciRNAs, compared to either linear counterparts.

As shown in Table 9, in the Ttr series, relative to the linear controls si-1 and si-3, the cyclic sciRNA compound si-4 had a 5-fold (transfection) and 4-fold (free uptake) loss of in vitro potency, correlating with the thermal destabilization observed in the Tₘ studies.

In the same context, the use of the 5′-(E)-vinylphosphonate VP modification of the antisense strands, a modification that can enhance the metabolic stability and Ago2 loading of siRNAs, were evaluated by preparing the control VP siRNA (si-2) and the cyclic sciRNA (si-5). By introducing the VP modification, the in vitro potency of the sciRNA conjugates was further enhanced and the potency gap between the linear and circular Ttr-targeting siRNAs was reduced to only 3-fold. There was an about 3-fold potency improvement triggered by the introduction of the VP modification as shown by comparison of the two control Ttr-targeting siRNAs (si-1 and si-2). The cyclic sciRNA with a VP-modified sense strand (si-5) was only 3-fold less potent than the VP-modified control with a linear sense strand (si-2), and displayed similar potency as the linear control (si-1).

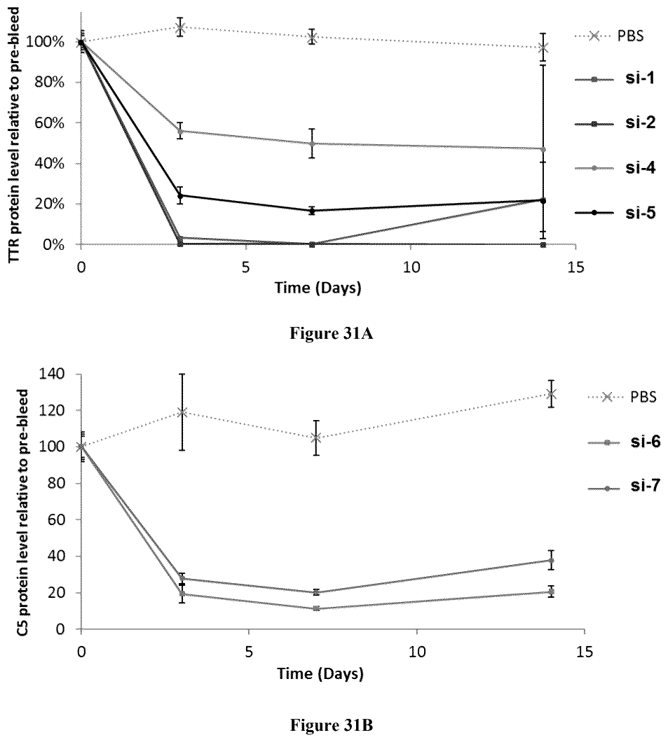

The GalNAc-sciRNAs activities were also evaluated in vivo. For this experiment, all GalNAc conjugates (including siRNAs and linear counterparts) were subcutaneously administered as a single dose (3 mg/kg on Day 0), and the serum levels of circulating TTR or C5 proteins were determined over time for each treatment group. The results of pharmacodynamics profiles after a single subcutaneous administration of linear GalNAc-siRNA and circular GalNAc-sciRNA conjugates in mice are shown in FIGS. 31A-31B. In FIGS. 31A-31B, both linear and circular designs showed a strong target inhibition in vivo. For the Ttr siRNA series, the data in mice mirrored the in vitro results as shown in Table 9, where a reduction of siRNA efficacy over the linear controls (si-1 and si-2) was observed with the sciRNA molecules (si-4 and si-5, FIG. 31A). For the series targeting C5, the identical 3 mg/kg dose of the linear (si-6) and circular (si-7) siRNA conjugates resulted in similar potency of reducing circulating C5 protein in mice (FIG. 31B), whereas at least an ˜8-fold potency loss was observed in vitro (Table 9).

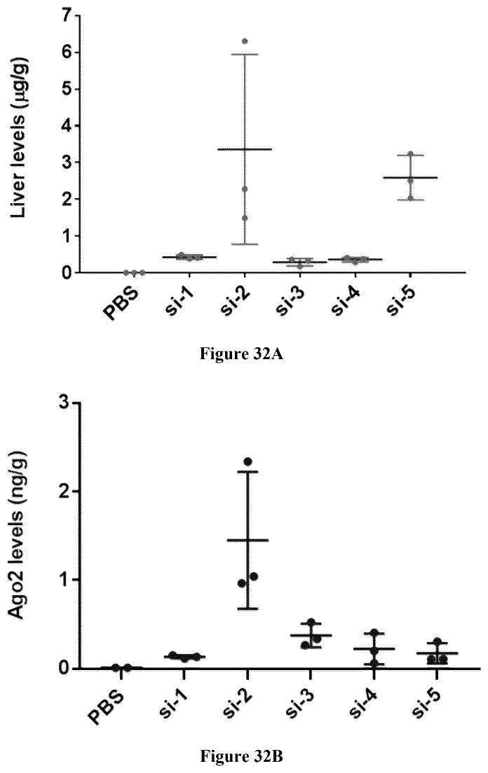

The in vivo metabolic stability of sciRNA compounds was further evaluated by measuring the total liver levels and Ago2-loaded levels, following a SC administration of a 3 mg/kg dose in mice. The results are shown in FIGS. 32A-32B. Mouse livers were harvested at Day 7 and treated to isolate and quantify the antisense strand of each siRNA by SL-RT QPCR. Relative liver amounts for the antisense strands measured for siRNAs si-1-si-5 are shown in FIG. 32A, showing that the addition of the metabolically stable VP group significantly increased the liver levels of both the corresponding linear siRNA (si-2) and circular sciRNA (si-5), over the non-VP siRNAs (˜5-fold more potent than respective controls with a linear sense strand si-1 and si-4, respectively). The whole liver levels between si-2 and si-5 were comparable (FIG. 32A), whereas their pharmacodynamics profiles in mice differed significantly (see FIG. 31A).

To evaluate the efficiency of Ago2 loading, Ago2 was immunoprecipitated from mouse whole liver lysate, and the antisense strand levels from Ago2 were extracted and measured, with the results shown in FIG. 32B. The VP modification in the linear siRNA enhanced Ago2 loading of the (si-2) antisense strand, relative to the non-VP siRNA (si-1) (FIG. 32B). The sciRNA VP counterpart (si-5) did not exhibit the same level of Ago2 loading enhancement, relative to the non-VP sciRNA (si-3) (FIG. 32B). A similar trend of reduction in Ago2 loading can be noted between the non-VP linear siRNA si-3 and the sciRNA si-4 (FIG. 32B). Linear siRNA (si-2) and circular sciRNA (si-5) exhibited similar antisense strand liver levels (FIG. 32A) but had different levels of Ago2-loaded antisense strand (FIG. 32B). This demonstrates that cyclic GalNAc-sciRNAs exhibited a metabolic stability similar to linear GalNAc-siRNAs, with a lower loading efficiency of antisense strands into Ago2.

These examples described the solid phase synthesis of a new class of fully chemically modified small circular interfering RNAs (sciRNAs) with a “click”-cyclized sense (S) strand, carrying a trivalent GalNAc ligand and an alkyne-functionalized linker. After completion of the sense strand synthesis, commercial bromo reagents were coupled at the 5′-end, and the bromide group of the linker was substituted with an azido group (Scheme I). After cleavage from the solid support, deprotection and purification, “click” reaction conditions allowed for the cyclization of the sense strands. Annealing of the circular sense strands with the corresponding complementary antisense (AS) strands yielded the GalNAc-conjugated sciRNA duplexes.

Physicochemical and pharmacological properties that these novel designs impart to GalNAc-sciRNAs were studied. Cyclization of a ˜20-nt oligonucleotide provided stability towards nucleases, but could reduce the Tₘ and thereby the thermal stability of the resulting RNA duplexes (see Tables 7 and 9). Annealing of shorter oligonucleotide targets (n=6-nt to 10-nt) to a 20-nt cyclic oligonucleotide showed increase in Tₘ values, demonstrating that a shorter linear target can be structurally well accommodated with a cyclic DNA strand. Furthermore, a triplex structure, where the cyclic 20-nt partner binds to two 10-nt strands can benefit from the cyclic partner being able to form Hoogsteen hydrogen bounds and lowers the entropy of the structure. The annealing of the 20-nt cyclic oligonucleotide with its 20-nt target displayed the same Tₘ value as that with the 10-nt target, suggesting that ˜10 nucleotides of the target were able to hybridize with the cyclic oligonucleotide. For DNA, longer nucleotide cycles may help form more stable circular duplexes. See Kumar et al., “Template-directed oligonucleotide strand ligation, covalent intramolecular DNA circularization and catenation using click chemistry,” J. Am. Chem. Soc., 129: 6859-64 (2007), which is incorporated herein by reference in its entirety.

The thermal melting and NMR analysis of the sciRNAs designed to form 21 base pairs indicate that when the sense strand has a rigid circular structure, a subset of the nucleobases form Watson-Crick base pairs. Fewer and broadened NMR signals were observed in spectra of sciRNAs than linear siRNAs suggesting that, in the sciRNAs, a heterogeneous mixture of partially base paired duplexes coexists in solution. The 21/23-nt sciRNA exhibited a circular sense strand structure, and when annealing with the linear antisense strand, a subset of the nucleobases formed Watson-Crick base pairs, which could impart a thermal destabilization to the duplex.

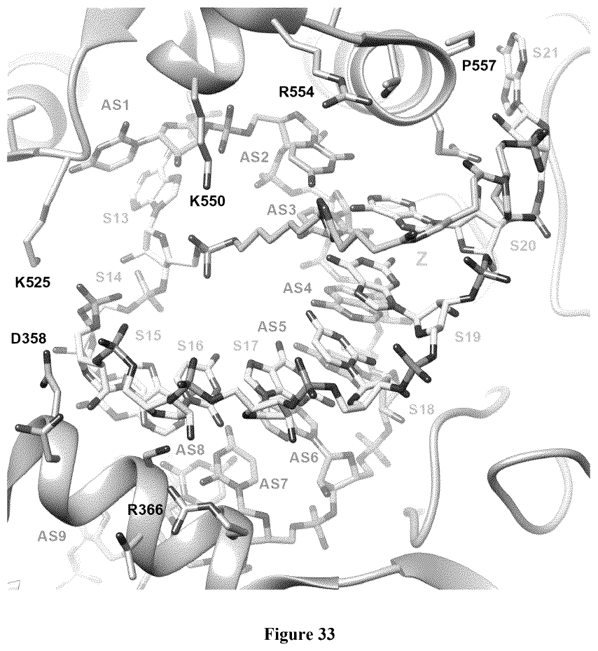

FIG. 33 shows a molecular model of an sciRNA:Ago2 complex based on the crystal structure of Ago2 bound to duplex RNA with seed region pairing. As shown in FIG. 33, the model of an sciRNA:Ago2 complex confirms that cyclization of the sense strand (S) with the Z linker maintained base pairing in the entire seed region (AS2:S20-AS8:S14). The crystal structure of Ago2 bound to AS and S RNA strands was used to construct the sciRNA scenario. See Schirle et al., “Structural basis for microRNA targeting,” Science, 346: 608-13 (2014), which is incorporated herein by reference in its entirety. Thus, the Z linker connects the 3′-terminal S strand nucleotide (A9 in the crystal structure, and labeled S21 in FIG. 33, consistent with the length of S strands listed in Table 9) and the first S strand residue visible in the crystal structure (A1, labeled S13 in the model). The latter residue was flipped around to complete the loop and close the circle, with the Z linker crossing the major groove of the S:AS duplex. Pairing across the entire seed region seen in the model is consistent with the increased Tₘ values observed for shorter oligonucleotides targeted with a 20-nt cyclic strand. Based on this model, a bigger circle with all 21 passenger strand nucleotides circularized by Z (+12 nt compared to the S13-S21 model depicted in FIG. 33) may be generated. Although, based on this model, the Z linker could presumably bridge across 8 or few pairs and, thus, after four or five more base pairs there would need a loop to connect the beginning of the sense strand back to the Z linker. The prolinol moiety adjacent to the 3′-phosphate of residue S21 in this model was oriented such that the hydroxyl group juts outwards, thus placing the GalNAc ligand on the surface of the complex and precluding potential clashes with Ago2 (FIG. 33).

The circular GalNAc-sciRNAs in this experiment demonstrated potent gene expression silencing in vitro and in vivo (Table 9, FIGS. 31A-31B). The thermal destabilization and duplex strain induced by the cyclization of the sense strand in sciRNAs showed an impact on the inherent potency of RNAi-mediated silencing, which is typically measured in vitro, especially when using transfection reagents for intracellular uptake.

However, the potency difference between linear GalNAc-siRNAs and circular GalNAc-sciRNAs was generally less pronounced when the constructs were evaluated in mice than in cell culture (FIGS. 31A-31B). The discrepancy between in vitro and in vivo potency could be the result of increased impact of metabolic stability on potency (efficacy in vivo) which outweighs intrinsic RNAi silencing potency. For example, in the Ttr series (FIG. 31A), the addition of the stabilizing VP group in the sciRNA (si-5) had a greater fold impact on in vivo potency than in vitro potency. In mice, at Day 14 post dose, the sciRNA with an antisense strand modified with VP had a potency equivalent to that of the linear non-VP-modified siRNA (si-1).

In rat plasma, neither sense nor antisense strands of sciRNAs or siRNAs were degraded in plasma at 8 hours. Considerable linearization of the circular sense strand of the sciRNA was observed after 24 hours of incubation. This indicates that the circular structure can act as a pro-drug, with hydrolysis (presumably of a phosphate group), resulting in a linear oligonucleotide. This hydrolysis did not occur when the compounds were incubated in rat liver homogenates. GalNAc-conjugated siRNA is typically rapidly absorbed into liver through efficient ASGPR-mediated uptake. Thus, linearization may have an impact on the in vivo potency due to rapid uptake of GalNAc-siRNA by hepatocytes.

The molecular model in FIG. 33 supports the loading of the sciRNA onto Ago2 by preserving the seed region nucleotide base pairing between the two RNA strands. The model also highlights important structural changes that may require the sciRNA duplex to flex and result in a thermodynamically destabilized duplex structure. In terms of intrinsic RNAi potency, the partial duplex structure shown in the model positions these sciRNA constructs somewhere between canonical siRNA duplexes and single-stranded siRNAs (ss-siRNAs). For the latter, loss of potency between 10- and 100-fold, compared to the siRNA duplex counterparts, has been reported (see Lima et al., “Single-stranded siRNAs activate RNAi in animals,” Cell, 150: 883-94 (2012); Prakash et al., “Lipid nanoparticles improve activity of single-stranded siRNA and gapmer antisense oligonucleotides in animals,” ACS Chem. Biol., 8: 1402-06 (2013), which are incorporated by reference in their entirety). To this date, no therapeutic development clinical candidate has been reported using the ss-siRNA platform. Moreover, the use of VP modifications in the sciRNAs provided potency benefit to the sciRNAs.

The results here also indicate that the sense strand played a role for RNAi activity for loading of the duplex into Ago2. The sense strand also had a role as being the carrier for the GalNAc ligand. The successful delivery of GalNAc-sciRNAs to hepatocytes demonstrates that the cyclic sense strand is complexed with the antisense strand despite the relative instability. Additionally, this confirms that the GalNAc ligand can effectively deliver RNAi therapeutics cargo when placed internally as well as terminally.

This work is the first example of partial-duplex sciRNA constructs that are functionally effective at pharmacologically and therapeutically relevant doses in vivo, useful for therapeutic siRNA molecules having enhanced potency resulting from increased metabolic stability and decreased off-target properties.

### Example 13: Synthesis of Circular Bis-sciRNA

Circular bis-sciRNAs were synthesized in this example, the sequences are shown in the table below.

| Multi | | | | | | | | |
|---|---|---|---|---|---|---|---|---|
| plex | Oligo | Sequence | | | Oligo | Sequence | | |
| ID | ID | (5′ to 3′)ª | Strand | Target | ID | (5′ to 3′) | Strand | Target |
| AM- | A- | usascug(Uhd)UfgGf | sense | CTNNB1/ | | | | |
| 206 | 2832990 | AfUfugauucgasasau | | SOD1 | | | | |
| | | uucsasuuuuAfaUfCf | | | | | | |
| | | CfucacucuasasZ84 | | | | | | |
| | A- | VPusUfsucgAfaUfC | antis | CTNNB1 | A- | VPusUfsuagAfgUf | antis | SOD1 |
| | 141361 | faaucCfaAfcaguasgs | | | 444402 | GfaggaUfuAfaaaug | | |
| | | C | | | | sasg | | |
| AM- | A- | usascuguUfgGfAfUf | sense | CTNNB1/ | | | | |
| 210 | 2832991 | ugauucgasasauuucsa | | SOD1 | | | | |
| | | suuuuAfaUfCfCfuca | | | | | | |
| | | cucuasasZ83L96 | | | | | | |
| | A- | VPusUfsucgAfaUfC | antis | CTNNB1 | A- | VPusUfsuagAfgUf | antis | SOD1 |
| | 141361 | faaucCfaAfcaguasgs | | | 444402 | GfaggaUfuAfaaaug | | |
| | | C | | | | sasg | | |

^{a}Chemistry modifications are indicated as follows: s, PS linkage; lower case nucleotides, 2′-OMe; upper case nucleotides followed by f (Uf, Gf, Cf, Af), 2′-F; VP, 5′-(E)-vinylphosphonate; (Uhd) is 2′-O-hexadecyl-uridine-3′-phosphate. For structures of Z83 and Z84, see Z in Scheme I, which denotes a cyclized sense strand.

Z83 indicates a cyclized sense strand via 3 internal cyclic click of Apy and Q301:

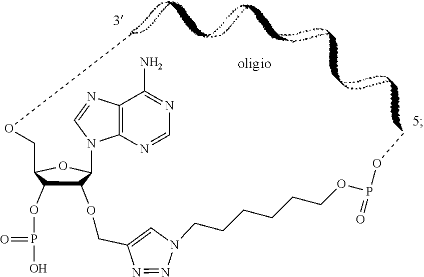

Z84 indicates a cyclized sense strand via 3 terminal cyclic click of Apy and Q301:

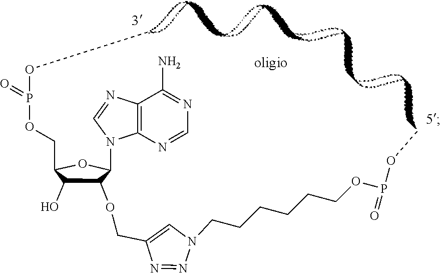

Apy refers to 2-O-propynyl-adenosine-3 phosphate:

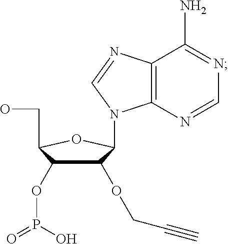

Q301 refers to 6-azidohexyl phosphate:

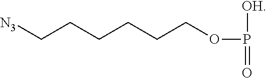

Oligonucleotides were synthesized on a MerMade-12 DNA/RNA synthesizer. Sterling solvents/reagents from Glen Research, 500-Å controlled pore glass (CPG) solid supports (Prime Synthesis), 2′-deoxy 3′-phosphoramidites (Thermo), and 2′-O-methyl (2′-OMe), 2′-deoxy-2′-fluoro (2′-F) ribonucleoside 3′-phosphoramidites (Hongene) were all used as received. The 2′-OMe-uridine-5′-bis-POM-(E) vinylphosphonate (VP) 3′-phosphoramidite (synthesized according to the procedures described in Parmar et al., “Facile synthesis, geometry, and 2′-substituent-dependent in vivo activity of 5′-(E)- and 5′-(Z)-vinylphosphonate-modified siRNA conjugates,” J. Med. Chem., 61: 734-44 (2018), which is incorporated herein by reference in its entirety) was dissolved to 0.15 M in 85% acetonitrile 15% dimethylformamide (DMF) and coupled using standard conditions on the synthesizer. GalNAc CPG support (L, Scheme I) was prepared and used as described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety. 5-Bromohexyl phosphoramidite (Glen Research, Cat #10-1946) was dissolved to 0.15 M in acetonitrile and coupled using standard conditions on the synthesizer. 3′-alkyne CPG support and 3′-alkyne hydroxyprolinol phosphoramidite (Y, Scheme I) was prepared and used as described in Jayaprakash et al., “Non-nucleoside building blocks for copper-assisted and copper-free click chemistry for the efficient synthesis of RNA conjugates,” Org. Lett., 12: 5410-13 (2010), which is incorporated herein by reference in its entirety. Low-water content acetonitrile was purchased from EMD Chemicals. DNA and RNA oligonucleotides were synthesized using modified synthesis cycles, based on those provided with the instrument. A solution of 0.6 M 5-(S-ethylthio)-1H-tetrazole in acetonitrile was used as the activator. The phosphoramidite solutions were 0.15 M in anhydrous acetonitrile with 15% DMF as a co-solvent for 2′-OMe uridine and cytidine. The oxidizing reagent was 0.02 M 12 in THF/pyridine/water. N,N-Dimethyl-N′-(3-thioxo-3H-1,2,4-dithiazol-5-yl)methanimidamide (DDTT), 0.09 M in pyridine, was used as the sulfurizing reagent. The detritylation reagent was 3% dichloroacetic acid (DCA) in dichloromethane (DCM).

After completion of the solid-phase syntheses (SPS), the CPG solid support was washed with 5% (v/v) piperidine in anhydrous acetonitrile three times with 5-minute holds after each flow. The support was then washed with anhydrous acetonitrile and dried with argon. The oligonucleotides were then incubated with 28-30% (w/v) NH₄OH at 35° C. for 20 hours. For VP-containing oligonucleotides, after completion of the SPS, the CPG solid support was incubated with 28-30% (w/v) NH₄OH, where 5% of (v/v) of diethylamine was added at 35° C. for 20 hours (see O'Shea et al., “An efficient deprotection method for 5′-[O, O-bis(pivaloyloxymethyl)]-(E)-vinylphosphonate containing oligonucleotides,” Tetrahedron, 74: 6182-86 (2018), which is incorporated herein by reference in its entirety). The solvent was collected by filtration and the support was rinsed with water prior to analysis. Oligonucleotide solutions of ˜1 OD₂₆₀ units/mL were used for analysis of the crudes, where 30-50 μL of solution were injected. LC/ESI-MS was performed on an Agilent 6130 single quadrupole LC/MS system using an XBridge C₈ column (2.1×50 mm, 2.5 μm) at 60° C. Buffer A consisted of 200 mM 1,1,1,3,3,3-hexafluoro-2-propanol (HFIP) and 16.3 mM triethylamine (TEA) in water, and buffer B was 100% methanol. A gradient from 0% to 40% of buffer B over 10 minutes followed by washing and recalibration at a flow rate of 0.70 mL/min. The column temperature was 75° C.

All oligonucleotides were purified and desalted, and further annealed to form GalNAc-siRNAs, based on the procedures described in Nair et al., “Multivalent N-acetylgalactosamine-conjugated siRNA localizes in hepatocytes and elicits robust RNAi-mediated gene silencing,” J. Am. Chem. Soc., 136: 16958-61 (2014), which is incorporated herein by reference in its entirety.

Circulation of Sense-Strand Oligonucleotides and Formation of sciRNA-GalNAc Conjugates—5′-Azido and “Click” Chemistry Synthesis for Circular Oligonucleotides.

To functionalize the sense strand, commercially available 5-bromohexyl phosphoramidite was coupled to the 5′ end during solid-phase synthesis. To provide the 6-azidohexyl click chemistry “handle”, 10 μmol of CPG loaded with 5′-(5-bromohexyl) modified oligonucleotides (see Lietard et al., “An efficient reagent for 5′-azido oligonucleotide synthesis,” Tetrahedron Lett., 48, 8795-98 (2007), which is incorporated herein by reference in its entirety) were suspended in 15 mL of an anhydrous DMF solution containing 130 mg of sodium azide and 300 mg of sodium iodide. The mixture was vigorously shaken at 65° C. for 75 minutes. After cooling down, the solution was filtered off and the CPG beads with the resulting 5′-(5-azidohexyl) solid-supported oligonucleotides were washed with DMF (2×10 mL) and dried under a stream of argon. The oligonucleotides were released from the solid support and purified and desalted as described above. The oligonucleotides were then dissolved in water to a concentration of ˜10 OD₂₆₀ units/mL. For a copper(I)-catalyzed alkyne-azide cycloaddition (CuAAC) “click” cyclization reaction yielding Z (Scheme I), 2 mL of the oligonucleotide solution (˜200 OD₂₆₀ units) were mixed with 2 mL of methanol, 1.1 mL of sodium L-ascorbate (0.1 mM) and 1.1 mL of copper sulfate (20 mM). The reaction mixture was placed into a microwave (MW) tube container, equipped with a stirring bar, and placed in a MW reactor for 40 minutes at 60° C. (power ˜8 W, stirring, cooling, P=0). This protocol is a modification of that described in Lietard et al., “New strategies for cyclization and bicyclization of oligonucleotides by click chemistry assisted by microwaves,” J. Org. Chem., 73, 191-200 (2008), which is incorporated herein by reference in its entirety. All cyclic oligonucleotides were purified and desalted, and further annealed with an antisense strand to form the GalNAc-sciRNA as described above.

### Evaluations of Knockdown in Mice of an Exemplary Circular Bis-sciRNA Via ICV Injection.

The knockdown of target mSOD1 and mCTNNB1 by an exemplary circular bis-sciRNA (AM-206) was assessed in mice in the central nervous system after a 100 μg ICV injection in the mice.

The experimental setup and evalution methods were the same as those discussed in Example 8. The sequences, chemical patterns, and linker of AM-206 are also shown in FIGS. 23A-23C.

The target mRNA knockdown levels in the mouse brain (FIG. 23D), liver and heart (FIG. 23E) were also compared to the linear bis-siRNA complexes and the mixed duplex delivery, and the results are shown in FIGS. 23D-23E.

### Evaluations of Knockdown in Rat of an Exemplary Circular Bis-sciRNA Via IT Injection.

The knockdown of target mSOD1 and mCTNNB1 by an exemplary circular bis-sciRNA (AM-206) was assessed in rat in the central nervous system after a 0.3 mg IT injection in the rat.

The experimental setup and evalution methods were the same as those discussed in Example 9. The sequences, chemical patterns, and linker of AM-206 are also shown in FIGS. 24A-24C.

The target mRNA knockdown levels in the thoracic spinal cord, frontal cortex, hippocampus, and striatum of rat were also compared to the linear bis-siRNA complexes and the mixed duplex delivery, and the results are shown in FIG. 24D.

### Analysis of Oligonucleotide Metabolism of the Circular Bis-sciRNA in Rat Brain Homogenates

The metabolic liabilities of an exemplary CNS-targeting circular bis-sciRNA were assessed after incubation of the circular bis-sciRNA in rat brain homogenate for 24 hours, and compared to the linear bis-siRNA complexes.

The experimental setup and evalution methods were the same as those discussed in Example 11, and the results are summarized in FIG. 26D.

1. A nucleic acid composition for modulating in the central nervous system (CNS) of a subject one or more target RNAs comprising one or more distinct target RNA sequences, the nucleic acid composition comprising a first double-stranded RNA (dsRNA) molecule and a single-stranded nucleic acid agent or a second dsRNA molecule, wherein:
the first dsRNA molecule and the single-stranded nucleic acid agent or second dsRNA molecule are connected together by a linker and do not overlap with each other,
the first dsRNA comprises at least one conjugated lipophilic moiety,
the single-stranded nucleic acid agent or second dsRNA molecule comprises at least one conjugated lipophilic moiety, and
each of the first dsRNA molecule and the single-stranded nucleic acid agent or second dsRNA molecule of said nucleic acid composition is capable of modulating the activity or expression of one or more target RNAs in a CNS tissue of the subject by at least 15% relative to an appropriate control.

2. The nucleic acid composition of claim 1 comprising the first dsRNA molecule and the second dsRNA molecule connected together by the linker.

3. The nucleic acid composition of claim 1, wherein at least one lipophilic moiety comprises a saturated or unsaturated C₄-C₃₀ hydrocarbon chain, and an optional functional group selected from the group consisting of hydroxyl, amine, carboxylic acid, sulfonate, phosphate, thiol, azide, and alkyne.

4. The nucleic acid composition of claim 1, wherein each lipophilic moiety comprises a saturated or unsaturated C₁₆ or C₂₂ hydrocarbon chain.

5. The nucleic acid composition of any one of claim 1, wherein the sense strand of the first dsRNA molecule is covalently linked to the sense strand of the second dsRNA molecule.

6. The nucleic acid composition of claim 1, wherein one or more of the first and second dsRNA molecules, if present, comprises a lipophilic moiety conjugated independently to position 6 of the sense strand of each dsRNA molecule, counting from the 5′-end of the sense strand of each dsRNA molecule.

7. The nucleic acid composition of claim 1, wherein one or more of the first and second dsRNA molecules, if present, comprises one or more lipophilic moieties conjugated independently to one or more of the following non-terminal positions: positions 5, 6, 7, 15, and 17 on the sense strand, and positions 15 and 17 on the antisense strand, counting from the 5′-end of each strand as position 1.

8. The nucleic acid composition of claim 1, wherein one or more of the first and second dsRNA molecules, if present, comprises a sense strand of 19-30 nucleotides in length and an antisense strand of 19-30 nucleotides in length.

9. The nucleic acid composition of claim 8, wherein each of the first dsRNA molecule and the second dsRNA molecule, if present, has a sense strand of 21-25 nucleotides in length an antisense strand of 21-25 nucleotides in length.

10. The nucleic acid composition of claim 1, wherein each dsRNA molecule of the nucleic acid composition comprises at least one modified nucleotide selected from the group consisting of a 2′-O-methyl modified nucleotide, a 2′-fluoro modified nucleotide, a nucleotide that includes a glycol nucleic acid (GNA) and a nucleotide that includes a vinyl phosphonate.

11. The nucleic acid composition of claim 1, wherein each dsRNA molecule comprises between two and eight phosphorothioate or methylphosphonate internucleotide linkages.

12. The nucleic acid composition of claim 1, wherein all or substantially all of the nucleotides of each dsRNA molecule comprise a modification selected from the group consisting of a 2′-O-methyl modification, a 2′-fluoro modification and a 2′-C₆-C₁₈ hydrocarbon chain modification.

13. The nucleic acid composition of claim 1, wherein the nucleic acid composition comprises two nucleic acid dsRNA molecules, wherein the sense strand of each dsRNA molecule is 21 nucleotides in length, the antisense strand of each dsRNA molecule is 23 nucleotides in length, the linker that connects the first dsRNA molecule and the single-stranded nucleic acid agent or second dsRNA molecule is a nucleic acid linker of three nucleotides in length that connects the sense strands of each dsRNA molecule, and the lipophilic moiety is conjugated to position 6 of the sense strand of each dsRNA molecule.

14. The nucleic acid composition of claim 1, wherein the linker is a bio-cleavable linker.

15. The nucleic acid composition of claim 1, wherein the linker comprises a moiety selected from the group consisting of DNA, RNA, disulfide, amide, functionalized monosaccharides or oligosaccharides of galactosamine, glucosamine, glucose, galactose, mannose, and combinations thereof.

16. The nucleic acid composition of claim 1, wherein the linker that connects the first dsRNA molecule and the single-stranded nucleic acid agent or second dsRNA molecule is a nucleic acid linker of between one and 15 nucleotides in length.

17. The nucleic acid composition of claim 16, wherein the linker is three nucleotides in length.

18. The nucleic acid composition of claim 1, wherein the linker that connects the first dsRNA molecule and the single-stranded nucleic acid agent or second dsRNA molecule is a nucleic acid linker comprising one or more nucleotides selected from the group consisting of 2′-O-methyl nucleotides, 2′-fluoro nucleotides, deoxyribonucleotides (dNTPs) and ribonucleotides.

19. The nucleic acid composition of claim 1, wherein the linker that connects the first dsRNA molecule and the single-stranded nucleic acid agent or second dsRNA molecule is a polynucleotide comprising one or more modifications selected from the group consisting of a 2′-O-methyl ribonucleotide modification, a 2′-fluoro-ribonucleotide modification, a 2′-5′-linked nucleotide with different 3′-modification (3′-ribo, 3′-O-methyl, 3′-deoxy, 3′-fluoro), a glycol nucleic acid (GNA) modification, a locked nucleic acid (LNA) modification, a hexanol nucleic acid (HNA) modification, an abasic ribose modification, an abasic deoxyribose modification, and an abasic hydroxyprolinol modification.

20. The nucleic acid composition of claim 1, wherein the linker that connects the first dsRNA molecule and the single-stranded nucleic acid agent or second dsRNA molecule is selected from the group consisting of

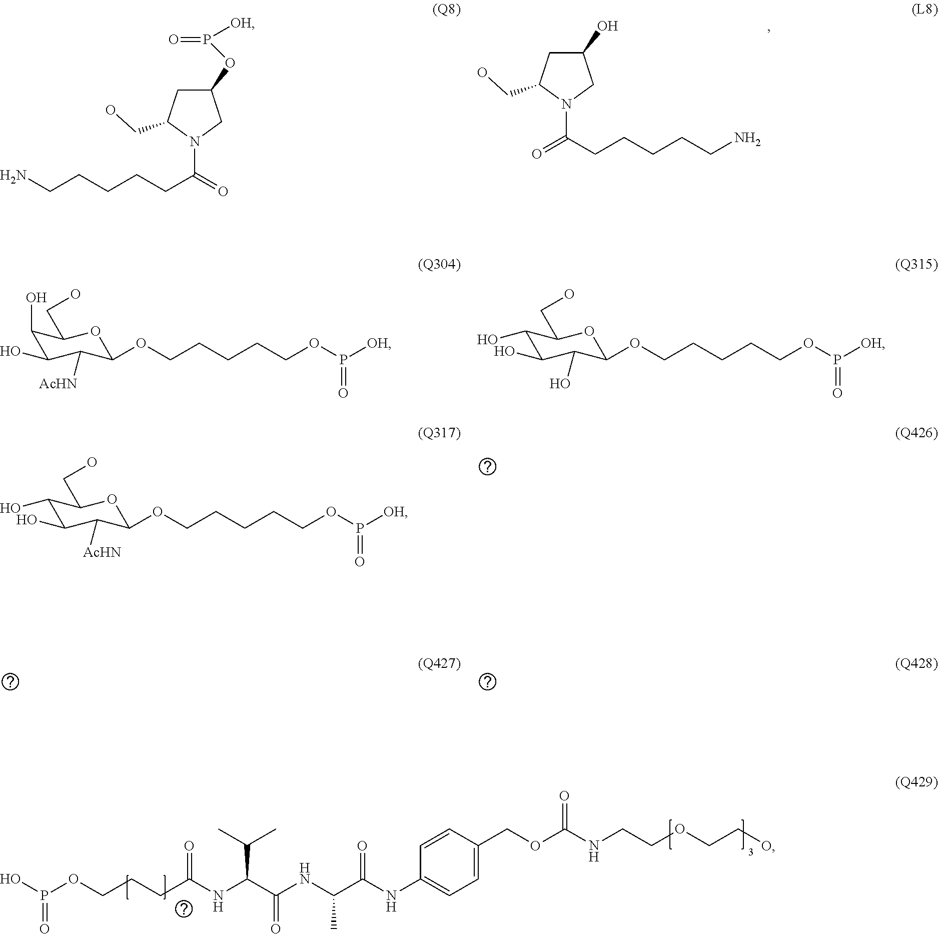

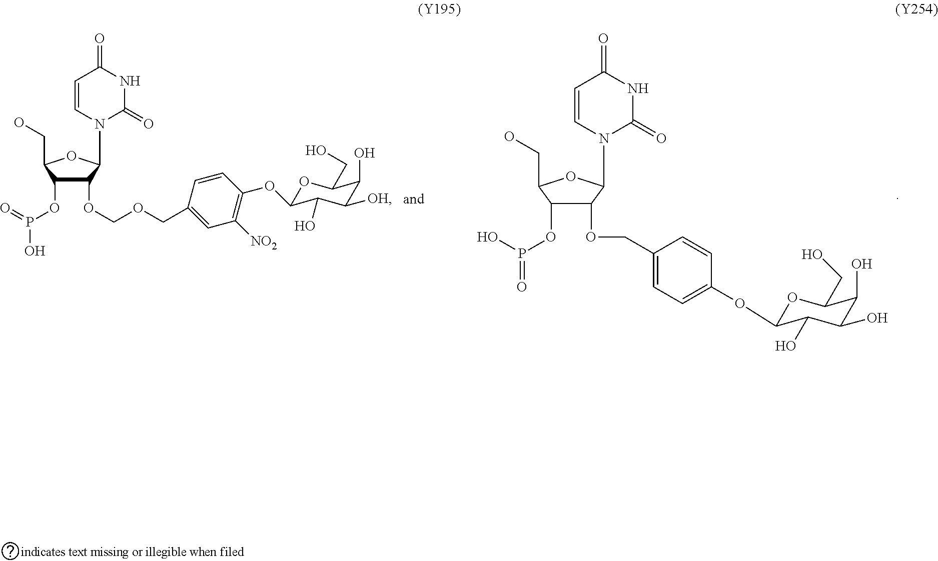

21. The nucleic acid composition of claim 1, wherein said nucleic acid composition modulates gene expression of at least two target nucleic acids by at least 75% each relative to when said first dsRNA molecule and said single-stranded nucleic acid agent or second dsRNA molecule are not connected together.

22. The nucleic acid composition of claim 1, wherein the first dsRNA molecule modulates gene expression of a first target nucleic acid and the single-stranded nucleic acid agent or second dsRNA molecule modulates gene expression of a second nucleic acid.

23. A method for modulating in the central nervous system (CNS) of a subject one or more target RNAs comprising one or more distinct target RNA sequences, the method comprising contacting the CNS cell of the subject with a nucleic acid composition comprising a first dsRNA molecule and a single-stranded nucleic acid agent or second dsRNA molecule, wherein the first dsRNA molecule and the single-stranded nucleic acid agent or second dsRNA molecule are connected together by a linker and do not overlap with each other, wherein each of the first dsRNA molecule and the second dsRNA molecule, if present, comprises at least one conjugated lipophilic moiety, and wherein said nucleic acid composition inhibits the activity or expression of the one or more target RNAs in the CNS cell of the subject by at least 15% each relative to an appropriate control, thereby modulating in the central nervous system (CNS) of the subject the one or more target RNAs comprising one or more distinct target RNA sequences.

24. A method for treating or preventing a CNS disease or disorder in a subject, the method comprising administering an injectate to the subject, wherein the injectate comprises a nucleic acid composition comprising at least a first dsRNA molecule and a single-stranded nucleic acid agent or second dsRNA molecule, wherein the first dsRNA molecule and the single-stranded nucleic acid agent or second dsRNA molecule are connected together by a linker and do not overlap with each other, wherein each of the at least first dsRNA molecule and the second dsRNA molecule, if present, comprises at least one conjugated lipophilic moiety, and wherein said nucleic acid composition inhibits the activity or expression of one or more target RNAs comprising one or more distinct target RNA sequences in a tissue of the CNS of the subject by at least 15% each relative to an appropriate control, thereby treating or preventing the CNS disease or disorder in the subject.

25. The method of claim 24, wherein the injectate is an ICV injectate or an intrathecal injectate.

26. A pharmaceutical composition for inhibiting expression of one or more target genes associated with a CNS disease or disorder, the pharmaceutical composition formulated for administration to the CNS of a subject and including a nucleic acid composition comprising at least a first dsRNA molecule and a single-stranded nucleic acid agent or second dsRNA molecule, wherein the first dsRNA molecule and the single-stranded nucleic acid agent or second dsRNA molecule are connected together by a linker and do not overlap with each other, wherein each of the at least first dsRNA molecule and the second dsRNA molecule, if present, comprises at least one conjugated lipophilic moiety, and wherein said nucleic acid composition is capable of inhibiting the activity or expression of one or more distinct target RNAs comprising one or more distinct target RNA sequences in a tissue of the CNS of the subject by at least 15% each relative to an appropriate control, and a pharmaceutically acceptable carrier.

27. A nucleic acid composition for modulating one or more distinct target RNAs comprising one or more distinct target RNA sequences in a central nervous system (CNS) tissue of a subject, according to the formula: 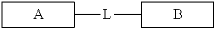
wherein
A is a first double-stranded RNA molecules (dsRNA);
B is a second double-stranded RNA molecules (dsRNA); and
L is a linker,
wherein
A and B do not overlap with each other, and each of A and B, independently, comprise at least one conjugated lipophilic moiety.

28-37. (canceled)

38. A small circular interfering RNA (sciRNA) for modulating one or more target mRNAs in the central nervous system (CNS) of a subject, comprising:
a first strand having at least 40 nucleotides in length and at least two first strand nucleotide sequences connected together by a bis-linker, each nucleotide sequence having about 18 to about 28 nucleotides in length, and
at least one second strand nucleotide sequence, having about 19 to about 23 nucleotides in length, annealed with at least one of the first strand nucleotide sequences;
wherein:
the first strand has a circular or substantially circular structure;
each of the first strand nucleotide sequences and the second strand nucleotide sequence(s) comprises at least one nucleic acid modification; and
the first strand nucleotide sequences or the second strand nucleotide sequence(s) comprise one or more ligands.

39-47. (canceled)
